(19) United States
(12) Patent Application Publication
(10) Pub. No.: US 2026/0272893 A1
(43) Pub. Date: Sep. 17, 2026

(54) COMPOUNDS AND COMPOSITIONS FOR TREATING CONDITIONS ASSOCIATED WITH CALCITONIN RECEPTOR AND/OR AMYLIN RECEPTOR ACTIVITY

(71) Applicant: Aconcagua Bio, Inc., South San Francisco, CA (US)

(72) Inventors: Zhenting GAO, Shanghai (CN); Hui LEI, Shanghai (CN); Cui LI, Shanghai (CN); Chunliang LU, Shanghai (CN); Ding XUE, Shanghai (CN); Haizhen ZHANG, Shanghai (CN); Zhuming ZHANG, Shanghai (CN); Evelyne HOUANG, New York, NY (US); Jian LIU, New York, NY (US); Zhe NIE, New York, NY (US); Anatoly RUVINSKY, New York, NY (US); Eric THERRIEN, New York, NY (US)

(21) Appl. No.: 19/458,586

(22) Filed: Jan. 23, 2026

Related U.S. Application Data
(63) Continuation of application No. PCT/US2025/015123, filed on Feb. 7, 2025.

(30) Foreign Application Priority Data
Feb. 8, 2024 (WO) PCT/CN2024/076921
Jun. 6, 2024 (WO) PCT/CN2024/097847
Nov. 19, 2024 (WO) PCT/CN2024/132993
Dec. 20, 2024 (WO) PCT/CN2024/141050

(51) Int. Cl. A61K 31/437; C07D 487/04

(52) U.S. Cl. CPC A61K 31/437; C07D 487/04

(57) ABSTRACT

The present disclosure provides compounds for modulating calcitonin receptor and/or amylin receptor activity, as well as pharmaceutical compositions comprising the compounds disclosed herein. Also provided are methods for treating a calcitonin receptor and/or amylin receptor associated disease or disorder.

### CROSS REFERENCE TO RELATED APPLICATIONS

The application claims the benefit of International Patent Application Number PCT/CN2024/076921, filed on Feb. 8, 2024, International Patent Application Number PCT/CN2024/097847, filed on Jun. 6, 2024, International Patent Application Number PCT/CN2024/132993, filed on Nov. 19, 2024, and International Patent Application Number PCT/CN2024/141050, filed Dec. 20, 2024, each of which is incorporated herein by reference in its entirety.

### FIELD

The present disclosure provides compounds for modulating calcitonin receptor and/or amylin receptor activity, as well as pharmaceutical compositions comprising the compounds disclosed herein. Also provided are methods for treating calcitonin receptor and/or amylin receptor associated diseases, disorders, and conditions.

### BACKGROUND

Calcitonin and amylin are hormones that interact with receptors within the same family to exert their effects on the human organism. Calcitonin, derived from thyroid C cells, is known for its inhibitory effect on osteoclasts. Calcitonin of mammalian origin promotes insulin sensitivity, while the more potent calcitonin extracted from salmon additionally inhibits gastric emptying, promotes gallbladder relaxation, increases energy expenditure and induces satiety as well as weight loss. Studies have also indicated that oral salmon calcitonin (sCT) exerts an insulin-sensitizing effect to improve glucose metabolism in obesity and type 2 diabetes. European Journal of Pharmacology, 2024, 737(7): 91-96.

Amylin receptors (AMYRs) are G protein-coupled receptors (GPCRs), which respond to the peptide hormones amylin and calcitonin. Amylin receptors are heterodimers comprising the calcitonin receptor, which is a G protein-coupled receptor, and one of three receptor-modifying proteins. Amylin, formed primarily in pancreatic islet β cells, is cosecreted with insulin in response to caloric intake. Patients with type 1 diabetes have lower baseline amylin serum concentrations, and amylin response to caloric intake is absent. Patients with type 2 diabetes requiring insulin also have a diminished amylin response to caloric intake, potentially related to the degree of β-cell impairment. Key physiologic functions of amylin in maintaining glucose homeostasis include suppressing glucagon release in response to caloric intake, delaying the rate of gastric emptying, and stimulating the satiety center in the brain to limit caloric intake.

The synthetic amylin analogue pramlintide is an approved treatment for diabetes mellitus as an adjunctive therapy to mealtime insulin which promotes better glycemic control and small but significant weight loss. AM833 (cagrilintide), an investigational novel long-acting acylated amylin analogue, acts as a non-selective amylin receptor agonist. This amylin receptor agonist can serve as an attractive novel treatment for obesity, resulting in reduction of food intake and significant weight loss in a dose-dependent manner. J Obes Metab Syndr. 2021; 30(4): 320-325.

Accordingly, modulators of the amylin and/or calcitonin receptor could be useful in treating various metabolic disorders, as well as inducing weight loss.

### SUMMARY

The present disclosure provides small molecule calcitonin and/or amylin receptor modulators (e.g., amylin-receptor agonists), as well as pharmaceutical compositions comprising the compounds disclosed herein. Also provided are methods for treating calcitonin receptor and/or amylin receptor associated diseases or disorders. It has been shown that calcitonin receptor activation is important for blood glucose regulation in diabetes, this is in addition to the known metabolic beneficial role of amylin receptor activation. Journal of Pharmacology and Experimental Therapeutics, 2020, 374(1), 74-83.

This disclosure also provides pharmaceutical compositions comprising one or more compounds as disclosed herein, or a stereoisomer or mixture of stereoisomers thereof, or pharmaceutically acceptable salt thereof, and a pharmaceutically acceptable excipient.

Also provided herein are pharmaceutical compositions comprising one or more compounds as disclosed herein, or a stereoisomer or mixture of stereoisomers thereof, or pharmaceutically acceptable salt thereof, and a pharmaceutically acceptable excipient.

Also provided herein are methods for treating or preventing a calcitonin receptor and/or an amylin receptor associated disease or disorder in a subject in need thereof, the method comprising administering to a subject in need thereof a therapeutically effective amount of a compound disclosed herein (e.g., a compound of Formula I or subformula thereof, or a pharmaceutically acceptable salt, stereoisomer, mixture of stereoisomers, or solvate thereof), or a pharmaceutical composition thereof. In some embodiments, the method further comprises administering to the subject, a therapeutically effective amount of one or more additional therapy or therapeutic agent to the patient, such as, but not limited to, an antidiabetic agent, an anti-obesity agent, a weight loss agent, a GLP-1 receptor agonist, an anti-emetic agent, an agent to treat non-alcoholic steatohepatitis (NASH), gastric electrical stimulation, dietary monitoring, physical activity, or a combination thereof.

In some embodiments, the calcitonin receptor and/or amylin receptor associated disease or disorder is a bone disorder, a metabolic disorder, pain, a neurodegenerative disease or disorder, a cardiovascular disease, or other disease or disorder.

In some embodiments, the calcitonin receptor and/or amylin receptor associated disease or disorder is a bone disorder, including, but not limited to, osteoporosis, Paget's disease, hypercalcemia, Sudeck's atrophy, polystatic fibrous dysplasia, intersemocostoclavicular ossification, osteogenesis imperfecta, osteopenia, periodontal disease or defect, osteolytic bone disease, metastatic bone disorder, or bone loss resulting from a malignancy, autoimmune arthritides, a breakage or fracture, or immobility or disuse.

In some embodiments, the calcitonin receptor and/or amylin receptor associated disease or disorder is pain, including, but not limited to, osteopathic pain, phantom limb pain, general pain, hyperalgesia, or pain associated with diabetic neuropathy.

In some embodiments, the calcitonin receptor and/or amylin receptor associated disease or disorder is a neurodegenerative disease or disorder, including, but not limited to, Alzheimer's disease.

In some embodiments, the calcitonin receptor and/or amylin receptor associated disease or disorder is a metabolic disorder, including, but not limited to, non-alcoholic fatty liver disease (NAFLD), non-alcoholic steatohepatitis (NASH), insulin dependent diabetes, non-insulin dependent diabetes, impaired glucose tolerance, obesity, syndrome X, or other diabetic complication.

In some embodiments, the calcitonin receptor and/or amylin receptor associated disease or disorder is include primary or secondary hyperthyroidism, endocrine disorder, conditions associated with inhibiting gastric secretion, gastrointestinal disorders, renal osteodystrophy, or male infertility.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

A dash (“—”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through a line in a structure indicates a specified point of attachment of a group. Unless chemically or structurally required, no directionality or stereochemistry is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about x” includes description of “x” Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂), and tert-butyl (i.e., —C(CH₃)₃), and “propyl” includes n-propyl (i.e., —(CH₂)₂CH₃), and isopropyl (i.e., —CH(CH₃)₂).

“Alkenyl” refers to an alkyl group containing at least one (e.g., 1-3, or 1) carbon-carbon double bond and having from 2 to 20 carbon atoms (i.e., C₂₋₂₀ alkenyl), 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include, e.g., ethenyl, propenyl, butadienyl (including 1,2-butadienyl, and 1,3-butadienyl).

“Alkynyl” refers to an alkyl group containing at least one (e.g., 1-3, or 1) carbon-carbon triple bond and having from 2 to 20 carbon atoms (i.e., C₂₋₂₀ alkynyl), 2 to 12 carbon atoms (i.e., C₂₋₁₂ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy, and 1,2-dimethylbutoxy.

“Alkoxyalkyl” refers to an alkyl group as defined above, wherein a hydrogen atom is replaced by an alkoxy group as defined herein.

“Haloalkyl” refers to an unbranched or branched alkyl group as defined above, wherein one or more (e.g., 1 to 6 or 1 to 3) hydrogen atoms are replaced by an independently selected halo group. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Haloalkoxy” refers to an alkoxy group as defined above, wherein one or more (e.g., 1 to 6, or 1 to 3) hydrogen atoms are replaced by an independently selected halo group.

“Haloalkoxyalkyl” refers to an alkyl group as defined above, wherein a hydrogen atom is replaced by a haloalkoxy group as defined herein.

“Hydroxyalkyl” refers to an alkyl group as defined above, wherein one or more (e.g., 1 to 6, or 1 to 3) hydrogen atoms are replaced by a hydroxy group.

“Cyanoalkyl” refers to an alkyl group as defined above, wherein one, or one or more (e.g., 1 to 6, or 1 to 3) hydrogen atoms are replaced by cyano.

“Alkylthio” refers to the group “alkyl-S—”.

“Acyl” refers to a group —C(O)R, wherein R is hydrogen, alkyl, cycloalkyl, heterocyclyl, aryl, heteroalkyl, or heteroaryl; each of which may be optionally substituted, as defined herein. Examples of acyl include formyl, acetyl, cyclohexylcarbonyl, cyclohexylmethyl-carbonyl, and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein, or R^{y} and R^{z} are taken together to form a cycloalkyl or heterocyclyl; each of which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl, or heteroaryl; each of which may be optionally substituted, as defined herein.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 14 ring carbon atoms (i.e., C₃₋₁₂ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋ₛ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl, and the like. Further, the term cycloalkyl is intended to encompass any non-aromatic ring which may be fused to an aryl ring, regardless of the attachment to the remainder of the molecule (e.g., 2,3-dihydro-1H-indenyl). Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Cycloalkylalkyl” refers to an alkyl group as defined above, wherein a hydrogen atom is replaced by a cycloalkyl group as defined herein.

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

“Imido” refers to a group —C(O)NR^{y}C(O)R^{z} or —N(C(O)R^{y})C(O)R^{z}, wherein R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein, or R^{y} and R^{z} are taken together to form a heterocyclyl which may be optionally substituted, as defined herein.

“Halogen” or “halo” refers to atoms occupying group VIIA of the periodic table, such as fluoro, chloro, bromo, or iodo.

“Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR—, —O—, —S—, —S(O)—, —S(O)₂—, and the like, where R is H, alkyl, aryl, cycloalkyl, heteroalkyl, heteroaryl or heterocyclyl, each of which may be optionally substituted. Examples of heteroalkyl groups include —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. As used herein, heteroalkyl include 1 to 10 carbon atoms, 1 to 8 carbon atoms, or 1 to 4 carbon atoms; and 1 to 3 heteroatoms, 1 to 2 heteroatoms, or 1 heteroatom.

“Heteroalkylene” refers to a divalent heteroalkyl group. “Heteroalkylene” groups must have at least one carbon and at least one heteroatomic group within the chain. The term “heteroalkylene” includes unbranched or branched saturated chain having carbon and heteroatoms. By way of example, 1, 2, or 3 carbon atoms may be independently replaced with the same or different heteroatomic group.

Heteroatomic groups include, but are not limited to, —NR^{y}—, —O—, —S—, —S(O)—, —S(O)₂—, and the like, wherein R is hydrogen, alkyl, alkenyl, alkynyl, cycloalkyl, heterocyclyl, aryl, heteroalkyl or heteroaryl; each of which may be optionally substituted, as defined herein. Examples of heteroalkylene groups include, e.g., —CH₂OCH₂—, —CH(CH₃)OCH₂—, —CH₂CH₂OCH₂—, —OCH₂—, —CH(CH₃)O—, —CH₂CH₂O—, —CH₂CH₂OCH₂CH₂OCH₂—, —CH₂CH₂OCH₂CH₂O—, —CH₂SCH₂—, —CH(CH₃)SCH₂—, —CH₂CH₂SCH₂—, —CH₂CH₂SCH₂CH₂SCH₂—, —SCH₂—, —CH(CH₃)S—, —CH₂CH₂S—, —CH₂CH₂SCH₂CH₂S—, —CH₂S(O)₂CH₂—, —CH(CH₃)S(O)₂CH₂—, —CH₂CH₂S(O)₂CH₂—, —CH₂CH₂S(O)₂CH₂CH₂OCH₂—, —CH₂NR^{y}CH₂—, —CH(CH₃)NR^{y}CH₂—, —CH₂CH₂NR^{y}CH₂—, —CH₂CH₂NR^{y}CH₂CH₂NR^{y}CH₂—, etc., where R^{y} is hydrogen, alkyl, alkenyl, alkynyl, cycloalkyl, heterocyclyl, aryl, heteroalkyl, or heteroaryl; each of which may be optionally substituted, as defined herein). As used herein, heteroalkylene includes 1 to 10 carbon atoms, 1 to 8 carbon atoms, or 1 to 4 carbon atoms; and 1 to 3 heteroatoms, 1 to 2 heteroatoms, or 1 heteroatom. As used herein, the term “heteroalkylene” does not include groups such as amides or other functional groups having an oxo present on one or more carbon atoms.

“Heteroaryl” refers to an aromatic group having a single ring or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl, benzotriazolyl, benzo[4,6]imidazo[1,2-a]pyridyl, carbazolyl, cinnolinyl, dibenzofuranyl, dibenzothienyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, thienyl, triazolyl, tetrazolyl, and triazinyl Examples of the fused-heteroaryl rings include, but are not limited to, benzo[d]thiazolyl, quinolinyl, isoquinolinyl, benzo[b]thienyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (═O) (e.g., —C(O)—, —S(O)—, —S(O)₂—, or —P(O)—) or N-oxide (—O⁻) moieties. Any non-aromatic ring or fused ring system containing at least one heteroatom and one non-aromatic ring is considered a heterocyclyl, regardless of the attachment to the remainder of the molecule. For example, fused ring systems such as 6,7-dihydro-5H-cyclopenta[b]pyridinyl, decahydroquinazolinyl, 1,2,3,4-tetrahydroquinazolinyl, and 5,6,7,8-tetrahydroquinazolinyl are heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiomorpholinyl, thiamorpholinyl, 1-oxo-thiomorpholinyl, and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

“Sulfonyl” refers to the group —S(O)₂R^{y}, where R^{y} is hydrogen, alkyl, alkenyl, alkynyl, cycloalkyl, heterocyclyl, aryl, heteroalkyl, or heteroaryl; each of which may be optionally substituted, as defined herein. Examples of sulfonyl are methylsulfonyl, ethylsulfonyl, phenylsulfonyl, and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl, or heteroaryl; each of which may be optionally substituted, as defined herein.

The terms “optional” or “optionally” means that the subsequently described event or circumstance may or may not occur, and that the description includes instances where said event or circumstance occurs and instances in which it does not. Also, the term “optionally substituted” refers to any one or more (e.g., 1 to 5, or 1 to 3) hydrogen atoms on the designated atom or group may or may not be replaced by a moiety other than hydrogen.

As used herein, the term “compound,” is meant to include any or all stereoisomers, geometric isomers, tautomers, and isotopically enriched analogs (e.g., deuterated analogs) of the structures depicted. Compounds herein identified by name or structure as one particular tautomeric form are intended to include other tautomeric forms unless otherwise specified.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Phamacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life, reduced dosage requirements, and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, aluminum, ammonium, calcium, and magnesium salts. Salts derived from organic bases include, but are not limited to, salts of NH₃, or primary, secondary, tertiary amines, such as salts derived from a N-containing heterocycle, a N-containing heteroaryl, or derived from an amine of formula N(R^{N})₃ (e.g., HN⁺(R^{N})₃ or (alkyl)N⁺(R^{N})₃) where each R^{N} is independently hydrogen, alkyl, alkenyl, alkynyl, haloalkyl, cycloalkyl, heterocyclyl, aryl, or heteroaryl, wherein each is optionally substituted, such as by one or more (e.g., 1-5 or 1-3) substituents (e.g., halo, cyano, hydroxy, amino, alkyl, alkenyl, alkynyl, haloalkyl, alkoxy, or haloalkoxy). Specific examples of suitable amines include, by way of example only, isopropylamine, trimethyl amine, diethyl amine, tri(isopropyl) amine, tri(n-propyl) amine, ethanolamine, 2-dimethylaminoethanol, piperazine, piperidine, morpholine, N-ethylpiperidine, and the like.

The term “substituted” means that any one or more hydrogen atoms on the designated atom or group is replaced with one or more substituents other than hydrogen, provided that the designated atom's normal valence is not exceeded. The one or more substituents include, but are not limited to, acyl, alkenyl, alkoxy, alkoxyalkyl, alkyl, alkylthio, alkynyl, amidino, amido, amino, aryl, azido, carbamoyl, carboxyl, carboxyl ester, cyano, cyanoalkyl, cycloalkyl, cycloalkylalkyl, guanidino, halo, haloalkoxy, haloalkoxyalkyl, haloalkyl, heteroalkyl, heteroaryl, heterocyclyl, hydrazino, hydroxy, hydroxyalkyl, imido, imino, nitro, oxo, sulfinyl, sulfonic acid, sulfonyl, thiocyanate, thiol, thione, or combinations thereof.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms) Such impermissible substitution patterns are well known to the skilled artisan. When used to modify a chemical group, the term “substituted” may describe other chemical groups defined herein. Unless specified otherwise, where a group is described as optionally substituted, any substituents of the group are themselves unsubstituted. For example, in some embodiments, the term “substituted alkyl” refers to an alkyl group having one or more substituents including hydroxy, halo, alkoxy, cycloalkyl, heterocyclyl, aryl, and heteroaryl. In other embodiments, the one or more substituents may be further substituted with halo, alkyl, haloalkyl, hydroxy, alkoxy, cycloalkyl, heterocyclyl, aryl, or heteroaryl, each of which is substituted. In other embodiments, the substituents may be further substituted with halo, alkyl, haloalkyl, alkoxy, hydroxy, cycloalkyl, heterocyclyl, aryl, or heteroaryl, each of which is unsubstituted.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

The term “pharmaceutically acceptable” as used herein indicates that the compound, or salt or composition thereof is compatible chemically and/or toxicologically with the other ingredients comprising a formulation and/or the subject being treated therewith

The term “administration” or “administering” refers to a method of giving a dosage of a compound or pharmaceutical composition to a vertebrate or invertebrate, including a mammal, a bird, a fish, or an amphibian. The method of administration can vary depending on various factors, e.g., the components of the pharmaceutical composition, the site of the disease, and the severity of the disease.

The terms “effective amount” or “effective dosage” or “pharmaceutically effective amount” or “therapeutically effective amount,” as used herein, refer to a sufficient amount of a chemical entity (e.g., a compound of Formula I, or a pharmaceutically acceptable salt, stereoisomer, mixture of stereoisomers, or solvate thereof) being administered which will relieve to some extent one or more of the symptoms of the disease or condition being treated, and can include curing the disease. “Curing” means that the symptoms of active disease are eliminated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. In some embodiments, a “therapeutically effective amount” of a compound as provided herein refers to an amount of the compound that is effective as a monotherapy or combination therapy.

The term “excipient” or “pharmaceutically acceptable excipient” means a pharmaceutically-acceptable material, composition, or vehicle, such as a liquid or solid filler, diluent, carrier, solvent, or encapsulating material. In some embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutical composition” refers to a mixture of a compound of Formula I, or a pharmaceutically acceptable salt, stereoisomer, mixture of stereoisomers, or solvate thereof as provided herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to, rectal, oral, intravenous, aerosol, parenteral, ophthalmic, pulmonary, and topical administration.

The term “calcitonin receptor and/or amylin receptor associated disease or disorder” as used herein is meant to include, without limitation, those diseases, disorders, or conditions in which activation of at least one calcitonin receptor (CTR) and/or amylin receptor (AMY) by calcitonin and/or amylin contributes to the symptomology or progression of the disease or disorder. These diseases or disorders may arise from one or more of a genetic, iatrogenic, immunological, infectious, metabolic, oncological, toxic, surgical, and/or traumatic etiology.

The terms “treat,” “treating,” and “treatment,” in the context of treating a disease, disorder, or condition, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The term “preventing”, as used herein, is the prevention of the onset, recurrence or spread, in whole or in part, of the disease or condition as described herein, or a symptom thereof.

The terms “subject,” “patient,” or “individual,” as used herein, are used interchangeably and refers to any animal, including mammals such as mice, rats, other rodents, rabbits, dogs, cats, swine, cattle, sheep, horses, primates, and humans. In some embodiments, the term refers to a subject, particularly a mammalian subject, for whom diagnosis, prognosis, or therapy is desired or needed. In some embodiments, the subject is a human. In some embodiments, the subject has experienced and/or exhibited at least one symptom of the disease, disorder, or condition to be treated and/or prevented.

The terms “treatment regimen” and “dosing regimen” are used interchangeably to refer to the dose and timing of administration of each therapeutic agent in a combination.

The term “pharmaceutical combination,” as used herein, refers to a pharmaceutical treatment resulting from the mixing or combining of more than one active ingredient and includes both fixed and non-fixed combinations of the active ingredients.

The term “combination therapy” as used herein refers to a dosing regimen of two different therapeutically active agents (i.e., the components or combination partners of the combination), wherein the therapeutically active agents are administered together or separately in a manner prescribed by a medical care taker or according to a regulatory agency as defined herein.

The term “modulate,” “modulating.” or “modulation,” as used herein, refers to a regulation or an adjustment (e.g., increase or decrease) and can include, for example agonism, partial agonism or antagonism.

### Compounds

Provided herein are compounds that are amylin modulators. In some embodiments, provided is a compound of Formula I:

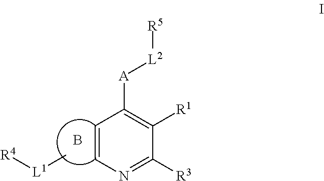

or a pharmaceutically acceptable salt, stereoisomer, mixture of stereoisomers, or solvate thereof, wherein:
A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroarylene of A is independently optionally substituted with one to five Z^{A};
Ring B is a C₅₋₆ cycloalkyl, 5- or 6-membered heterocyclyl, C₆ aryl, or 5- or 6-membered heteroaryl; wherein the C₅₋₆ cycloalkyl, 5- or 6-membered heterocyclyl, C₆ aryl, or 5- or 6-membered heteroaryl is optionally substituted with one to three R^{B};
each R^{B} is independently selected from halo, hydroxy, —NH₂—, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, C₁₋₃ heteroalkylene, or cyclopropylene; wherein the C₁₋₃ alkylene, C₂₋₃ alkenylene, C₂₋₃ alkynylene, or C₁₋₃ heteroalkylene, or cyclopropylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR₂ₐS(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
R¹ is cyano, —CH₂OH, —C(O)H, —C(O)NR^{1a}R^{1b}, —C(S)NR^{1a}R^{1b}, —S(O)₂R², —S(O)(NR⁶)R², or —P(O)R⁷R²;
R² is —NR^{1a}R^{1b}, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl of R² is optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and Cia haloalkoxy;
or R² and R⁶, or R² and R⁷ together with the atom to which they are attached, form a heterocyclyl optionally substituted with one to five Z²;
each R^{1a} and R^{1b} is independently hydrogen, C₁₋₃ alkyl, C₂₋₃ alkenyl, C₂₋₃ alkynyl, C₁₋₃ haloalkyl, C₃₋₆ cycloalkyl, or 4-6 membered heterocyclyl; wherein each C₁₋₃ alkyl, C₂₋₃ alkenyl, C₂₋₃ alkynyl, C₁₋₃ haloalkyl, C₃₋₆ cycloalkyl, or 4-6 membered heterocyclyl of R^{1a} and R^{1b} is independently optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R^{1a} and R^{1b} are taken together with the atoms to which they are attached to form 4-6 membered heterocyclyl independently optionally substituted by one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b};
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
R⁶ is hydrogen, C₁₋₃ alkyl, C₁₋₃ haloalkyl, C₃₋₆ cycloalkyl, or 4 to 6-membered heterocyclyl; wherein the C₁₋₃ alkyl, C₁₋₃ haloalkyl, C₃₋₆ cycloalkyl, or 4 to 6-membered heterocyclyl is optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
R⁷ is hydroxy, C₁₋₃ alkoxy, C₁₋₃ haloalkoxy, C₁₋₃ alkyl, or C₁₋₃ haloalkyl;
each Z^{A}, Z², Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z², Z^{2a}, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;

provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, provided is a compound of Formula I:

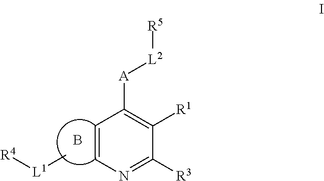

or a pharmaceutically acceptable salt, stereoisomer, mixture of stereoisomers, or solvate thereof, wherein:
A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroaryl of A is independently optionally substituted with one to five Z^{A};
Ring B is a C₅₋₆ cycloalkyl, 5- or 6-membered heterocyclyl, C₆ aryl, or 5- or 6-membered heteroaryl; wherein the C₅₋₆ cycloalkyl, 5- or 6-membered heterocyclyl, C₆ aryl, or 5- or 6-membered heteroaryl is optionally substituted with one to three R^{B};
each R^{B} is independently selected from halo, hydroxy, —NH₂—, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, or C₁₋₃ heteroalkylene; wherein the C₁₋₃ alkylene, C₂₋₃ alkenylene, C₂₋₃ alkynylene, or C₁₋₃ heteroalkylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
R¹ is cyano, —CH₂OH, —C(O)H, —C(O)NR^{1a}R^{1b}, —C(S)NR^{1a}R^{1b}, —S(O)₂R², —S(O)(NR⁶)R², or —P(O)R⁷R²;
R² is —NR^{1a}R^{1b}, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl of R² is optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R² and R⁶, or R² and R⁷, together with the atom to which they are attached, form a heterocyclyl optionally substituted with one to five Z²;
each R^{1a} and R^{1b} is independently hydrogen, C₁₋₃ alkyl, C₂₋₃ alkenyl, C₂₋₃ alkynyl, C₁₋₃ haloalkyl, C₃₋₆ cycloalkyl, or 4-6 membered heterocyclyl; wherein each C₁₋₃ alkyl, C₂₋₃ alkenyl, C₂₋₃ alkynyl, C₁₋₃ haloalkyl, C₃₋₆ cycloalkyl, or 4-6 membered heterocyclyl of R^{1a} and R^{1b} is independently optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R^{1a} and R^{1b} are taken together with the atoms to which they are attached to form 4-6 membered heterocyclyl independently optionally substituted by one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b},
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z³;
R⁶ is hydrogen, C₁₋₃ alkyl, C₁₋₃ haloalkyl, C₃₋₆ cycloalkyl, or 4 to 6-membered heterocyclyl; wherein the C₁₋₃ alkyl, C₁₋₃ haloalkyl, C₃₋₆ cycloalkyl, or 4 to 6-membered heterocyclyl is optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
R⁷ is hydroxy, C₁₋₃ alkoxy, C₁₋₃ haloalkoxy, C₁₋₃ alkyl, or C₁₋₃ haloalkyl;
each Z^{A}, Z², Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z², Z^{2a}, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano.

In some embodiments, provided is a compound of Formula I:

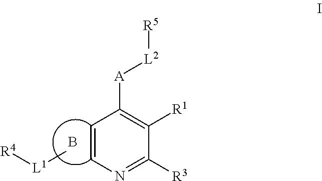

or a pharmaceutically acceptable salt, stereoisomer, mixture of stereoisomers, or solvate thereof, wherein:
A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroaryl of A is independently optionally substituted with one to five Z^{A};
Ring B is a C₅₋₆ cycloalkyl, 5- or 6-membered heterocyclyl, C₆ aryl, or 5- or 6-membered heteroaryl; wherein the C₅₋₆ cycloalkyl, 5- or 6-membered heterocyclyl, C₆ aryl, or 5- or 6-membered heteroaryl is optionally substituted with one to three R^{B};
each R^{B} is independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, or C₁₋₃ heteroalkylene; wherein the C₁₋₃ alkylene, C₂₋₃ alkenylene, C₂₋₃ alkynylene, or C₁₋₃ heteroalkylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR²C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
R¹ is cyano, —CH₂OH, —C(O)H, —C(O)NR^{1a}R^{1b}, —C(S)NR^{1a}R^{1b}, —S(O)₂R², —S(O)(NR⁶)R², or —P(O)R⁷R²;
R² is —NR^{1a}R^{1b}, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl of R² is optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R² and R⁶, or R² and R⁷, together with the atom to which they are attached, form a heterocyclyl optionally substituted with one to five Z²;
each R^{1a} and R^{1b} is independently hydrogen, C₁₋₃ alkyl, C₂₋₃ alkenyl, C₂₋₃ alkynyl, C₁₋₃ haloalkyl, C₃₋₆ cycloalkyl, or 4-6 membered heterocyclyl; wherein each C₁₋₃ alkyl, C₂₋₃ alkenyl, C₂₋₃ alkynyl, C₁₋₃ haloalkyl, C₃₋₆ cycloalkyl, or 4-6 membered heterocyclyl of R^{1a} and R^{1b} is independently optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R^{1a} and R^{1b} are taken together with the atoms to which they are attached to form 4-6 membered heterocyclyl independently optionally substituted by one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b};
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
R⁶ is hydrogen, C₁₋₃ alkyl, C₁₋₃ haloalkyl, C₃₋₆ cycloalkyl, or 4 to 6-membered heterocyclyl; wherein the C₁₋₃ alkyl, C₁₋₃ haloalkyl, C₃₋₆ cycloalkyl, or 4 to 6-membered heterocyclyl is optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
R⁷ is hydroxy, C₁₋₃ alkoxy, C₁₋₃ haloalkoxy, C₁₋₃ alkyl, or C₁₋₃ haloalkyl;
each Z^{A}, Z², Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z², Z^{2a}, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkonyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, R¹ is —C(O)NR^{1a}R^{1b}.

In some embodiments, R¹ is —CH₂OH, —C(O)H, or —C(O)NH₂.

In some embodiments, R¹ is —C(O) NH₂.

In some embodiments, R³ is C₁₋₆ alkyl. In some embodiments, R³ is isobutyl.

In some embodiments, provided is a compound represented by Formula IA:

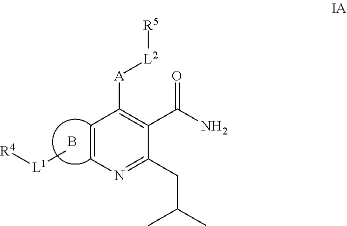

or a pharmaceutically acceptable salt, stereoisomer, mixture of stereoisomers, or solvate thereof, wherein Ring B, L¹, L², A, R⁴, and R⁵, are each independently as defined herein.

In some embodiments, provided is a compound represented by Formula IA:

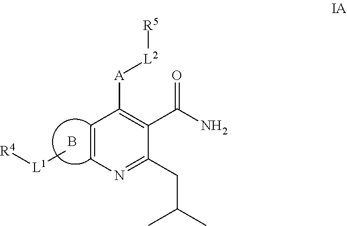

or a pharmaceutically acceptable salt, stereoisomer, mixture of stereoisomers, or solvate thereof, wherein:
Ring B is a C₅₋₆ cycloalkyl, 5- or 6-membered heterocyclyl, C₆ aryl, or 5- or 6-membered heteroaryl; wherein the C₅₋₆ cycloalkyl, 5- or 6-membered heterocyclyl, C₆ aryl, or 5- or 6-membered heteroaryl is optionally substituted with one to three R^{B};
each R^{B} is independently selected from halo, hydroxy, —NH₂—, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, C₁₋₃ heteroalkylene, or cyclopropylene; wherein the C₁₋₃ alkylene, C₂₋₃ alkenylene, C₂₋₃ alkynylene, or C₁₋₃ heteroalkylene, or cyclopropylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
each Z^{A}, Z^{2a}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkonyl, C₂₋₆ alkynyl, C₁₋₆ haloalkyl, C₃₋₁₀ cycloalkyl, heterocyclyl, aryl, or heteroaryl of Z^{A}, Z², Z^{2a}, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkonyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, Ring B is C₅₋₆ cycloalkyl, 5- or 6-membered heterocyclyl, or 5- or 6-membered heteroaryl; wherein the C₅₋₆ cycloalkyl, 5- or 6-membered heterocyclyl, C₆ aryl, or 5- or 6-membered heteroaryl is optionally substituted with one to three R^{B}.

In some embodiments, Ring B is 5- or 6-membered heterocyclyl optionally substituted with one R^{B}. In some embodiments, Ring B is 5- or 6-membered heterocyclyl containing an oxo, optionally substituted with one R^{B}.

In some embodiments, provided is a compound represented by Formula II:

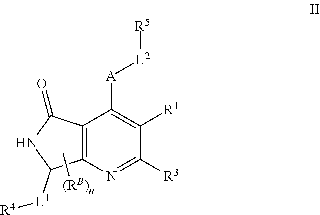

or a pharmaceutically acceptable salt, stereoisomer, mixture of stereoisomers, or solvate thereof, wherein R^{B}, L¹, L², A, R¹, R³, R⁴, and R⁵, are each independently as defined herein, and n is 0 or 1.

In some embodiments, provided is a compound represented by Formula II:

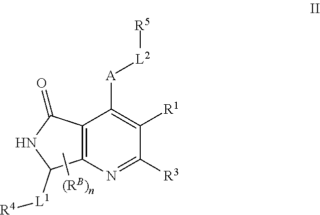

or a pharmaceutically acceptable salt, stereoisomer, mixture of stereoisomers, or solvate thereof, wherein:
n is 0 or 1;
A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroarylene of A is independently optionally substituted with one to five Z^{A};
R^{B} is selected from halo, hydroxy, —NH₂—, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, C₁₋₃ heteroalkylene, or cyclopropylene; wherein the C₁₋₃ alkylene, C₂₋₃ alkenylene, C₂₋₃ alkynylene, or C₁₋₃ heteroalkylene, or cyclopropylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR²⁰C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
R¹ is cyano, —CH₂OH, —C(O)H, —C(O)NR^{1a}R^{1b}, —C(S)NR^{1a}R^{1b}, —S(O)₂R², —S(O)(NR⁶)R², or —P(O)R⁷R²;
R² is —NR^{1a}R^{1b}, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl of R² is optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R² and R⁶, or R² and R⁷, together with the atom to which they are attached, form a heterocyclyl optionally substituted with one to five Z²;
each R^{1a} and R^{1b} is independently hydrogen, C₁₋₃ alkyl, C₂₋₃ alkenyl, C₂₋₃ alkynyl, C₁₋₃ haloalkyl, C₃₋₆ cycloalkyl, or 4-6 membered heterocyclyl; wherein each C₁₋₃ alkyl, C₂₋₃ alkenyl, C₂₋₃ alkynyl, C₁₋₃ haloalkyl, C₃₋₆ cycloalkyl, or 4-6 membered heterocyclyl of R^{1a} and R^{1b} is independently optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R^{1a} and R^{1b} are taken together with the atoms to which they are attached to form 4-6 membered heterocyclyl independently optionally substituted by one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b};
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
R⁶ is hydrogen, C₁₋₃ alkyl, C₁₋₃ haloalkyl, C₃₋₆ cycloalkyl, or 4 to 6-membered heterocyclyl; wherein the C₁₋₃ alkyl, C₁₋₃ haloalkyl, C₃₋₆ cycloalkyl, or 4 to 6-membered heterocyclyl is optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
R⁷ is hydroxy, C₁₋₃ alkoxy, C₁₋₃ haloalkoxy, C₁₋₃ alkyl, or C₁₋₃ haloalkyl;
each Z^{A}, Z², Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z², Z^{2a}, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, provided is a compound represented by Formula IIA:

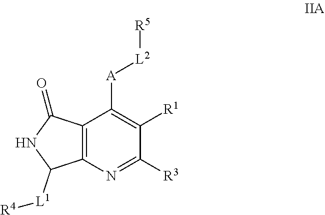

or a pharmaceutically acceptable salt, stereoisomer, mixture of stereoisomers, or solvate thereof, wherein L¹, L², A, R¹, R³, R⁴, and R⁵, are each independently as defined herein.

In some embodiments, provided is a compound represented by Formula IIB:

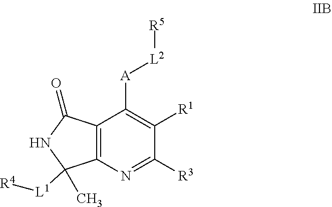

or a pharmaceutically acceptable salt, stereoisomer, mixture of stereoisomers, or solvate thereof, wherein L¹, L², A, R¹, R³, R⁴, and R⁵, are each independently as defined herein.

In some embodiments, provided is a compound represented by Formula IIC:

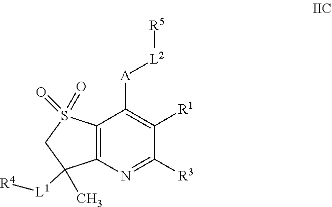

or a pharmaceutically acceptable salt, stereoisomer, mixture of stereoisomers, or solvate thereof, wherein L¹, L², A, R¹, R³, R⁴, and R⁵, are each independently as defined herein.

In some embodiments, provided is a compound represented by Formula IIA, IIB, or IIC:

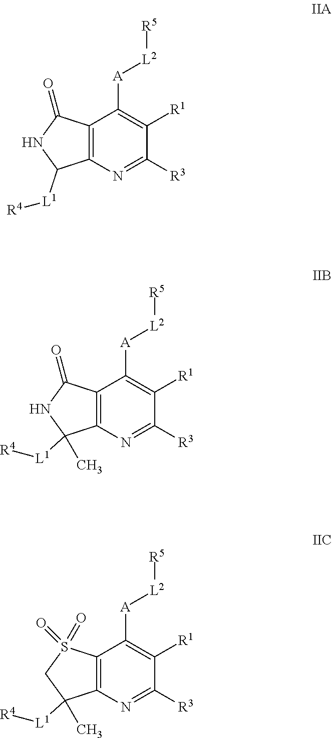

or a pharmaceutically acceptable salt, stereoisomer, mixture of stereoisomers, or solvate thereof, wherein:
A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroarylene of A is independently optionally substituted with one to five Z^{A};
L¹ is C₁₋₃ alkylene, C₂₋₃ alkenylene, C₂₋₃ alkynylene, C₁₋₃ heteroalkylene, or cyclopropylene; wherein the C₁₋₃ alkylene, C₂₋₃ alkenylene, C₂₋₃ alkynylene, or C₁₋₃ heteroalkylene, or cyclopropylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
R¹ is cyano, —CH₂OH, —C(O)H, —C(O)NR^{1a}R^{1b}, —C(S) NR^{1a}R^{1b}, —S(O)₂R², —S(O)(NR⁶)R², or —P(O)R⁷R²;
R² is —NR^{1a}R^{1b}, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl of R² is optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R² and R⁶, or R² and R⁷, together with the atom to which they are attached, form a heterocyclyl optionally substituted with one to five Z²;
each R^{1a} and R^{1b} is independently hydrogen, C₁₋₃ alkyl, C₂₋₃ alkenyl, C₂₋₃ alkynyl, C₁₋₃ haloalkyl, C₃₋₆ cycloalkyl, or 4-6 membered heterocyclyl; wherein each C₁₋₃ alkyl, C₂₋₃ alkenyl, C₂₋₃ alkynyl, C₁₋₃ haloalkyl, C₃₋₆ cycloalkyl, or 4-6 membered heterocyclyl of R^{1a} and R^{1b} is independently optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R^{1a} and R^{1b} are taken together with the atoms to which they are attached to form 4-6 membered heterocyclyl independently optionally substituted by one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b};
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
R⁶ is hydrogen, C₁₋₃ alkyl, C₁₋₃ haloalkyl, C₃₋₆ cycloalkyl, or 4 to 6-membered heterocyclyl; wherein the C₁₋₃ alkyl, C₁₋₃ haloalkyl, C₃₋₆ cycloalkyl, or 4 to 6-membered heterocyclyl is optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
R⁷ is hydroxy, C₁₋₃ alkoxy, C₁₋₃ haloalkoxy, C₁₋₃ alkyl, or C₁₋₃ haloalkyl;
each Z^{A}, Z², Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z², Z^{2a}, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹;
each R²⁰ and R²¹ is independently hydrogen, C₁₋₆ alkyl, C₂₋₆ alkonyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}, or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, provided is a compound represented by Formula III:

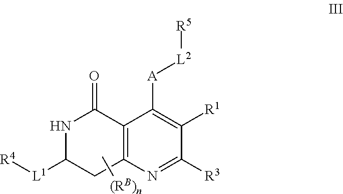

or a pharmaceutically acceptable salt, stereoisomer, mixture of stereoisomers, or solvate thereof, wherein R^{B}, L¹, L², A, R¹, R³, R⁴, and R⁵, are each independently as defined herein, and n is 0 or 1.

In some embodiments, provided is a compound represented by Formula III:

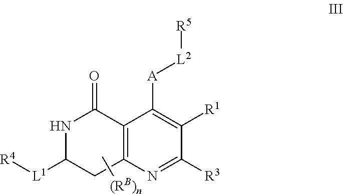

or a pharmaceutically acceptable salt, stereoisomer, mixture of stereoisomers, or solvate thereof, wherein:
n is 0 or 1;
A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroarylene of A is independently optionally substituted with one to five Z^{A};
R^{B} is selected from halo, hydroxy, —NH₂—, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, C₁₋₃ heteroalkylene, or cyclopropylene; wherein the C₁₋₃ alkylene, C₂₋₃ alkenylene, C₂₋₃ alkynylene, or C₁₋₃ heteroalkylene, or cyclopropylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
R¹ is cyano, —CH₂OH, —C(O)H, —C(O)NR^{1a}R^{1b}, —C(S) NR^{1a}R^{1b}, —S(O)₂R², —S(O)(NR⁶)R², or —P(O)R⁷R²;
R² is —NR^{1a}R^{1b}, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl of R² is optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R² and R⁶, or R² and R⁷, together with the atom to which they are attached, form a heterocyclyl optionally substituted with one to five Z²;
each R^{1a} and R^{1b} is independently hydrogen, C₁₋₃ alkyl, C₂₋₃ alkenyl, C₂₋₃ alkynyl, C₁₋₃ haloalkyl, C₃₋₆ cycloalkyl, or 4-6 membered heterocyclyl; wherein each C₁₋₃ alkyl, C₂₋₃ alkenyl, C₂₋₃ alkynyl, C₁₋₃ haloalkyl, C₃₋₆ cycloalkyl, or 4-6 membered heterocyclyl of R^{1a} and R^{1b} is independently optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R^{1a} and R^{1b} are taken together with the atoms to which they are attached to form 4-6 membered heterocyclyl independently optionally substituted by one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b};
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
R⁶ is hydrogen, C₁₋₃ alkyl, C₁₋₃ haloalkyl, C₃₋₆ cycloalkyl, or 4 to 6-membered heterocyclyl; wherein the C₁₋₃ alkyl, C₁₋₃ haloalkyl, C₃₋₆ cycloalkyl, or 4 to 6-membered heterocyclyl is optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
R⁷ is hydroxy, C₁₋₃ alkoxy, C₁₋₃ haloalkoxy, C₁₋₃ alkyl, or C₁₋₃ haloalkyl;
each Z^{A}, Z², Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z², Z^{2a}, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, provided is a compound represented by Formula IIIA:

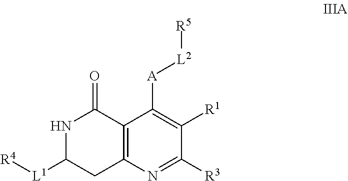

or a pharmaceutically acceptable salt, stereoisomer, mixture of stereoisomers, or solvate thereof, wherein L¹, L², A, R¹, R³, R⁴, and R⁵, are each independently as defined herein.

In some embodiments, provided is a compound represented by Formula IIIB:

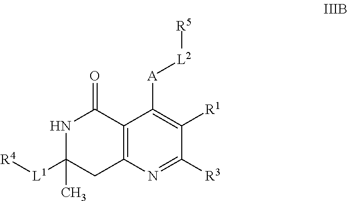

or a pharmaceutically acceptable salt, stereoisomer, mixture of stereoisomers, or solvate thereof, wherein L¹, L², A, R¹, R³, R⁴, and R⁵, are each independently as defined herein.

In some embodiments, provided is a compound represented by Formula IIIA or IIIB:

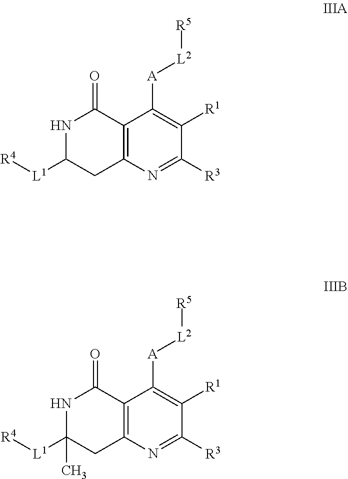

or a pharmaceutically acceptable salt, stereoisomer, mixture of stereoisomers, or solvate thereof, wherein:
A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroarylene of A is independently optionally substituted with one to five Z^{A};
L¹ is C₁₋₃ alkylene, C₂₋₃ alkenylene, C₂₋₃ alkynylene, C₁₋₃ heteroalkylene, or cyclopropylene; wherein the C₁₋₃ alkylene, C₂₋₃ alkenylene, C₂₋₃ alkynylene, or C₁₋₃ heteroalkylene, or cyclopropylene of L′ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
R¹ is cyano, —CH₂OH, —C(O)H, —C(O)NR^{1a}R^{1b}, —C(S)NR^{1a}R^{1b}, —S(O)₂R², —S(O)(NR⁶)R², or —P(O)R⁷R²;
R² is —NR^{1a}R^{1b}, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkonyl, C₂₋₆ alkynyl, C₃₋₁₀ cycloalkyl, heterocyclyl, aryl, or heteroaryl of R² is optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy; or R² and R⁶, or R² and R⁷, together with the atom to which they are attached, form a heterocyclyl optionally substituted with one to five Z²;
each R^{1a} and R^{1b} is independently hydrogen, C₁₋₃ alkyl, C₂₋₃ alkenyl, C₂₋₃ alkynyl, C₁₋₃ haloalkyl, C₃₋₆ cycloalkyl, or 4-6 membered heterocyclyl; wherein each C₁₋₃ alkyl, C₂₋₃ alkenyl, C₂₋₃ alkynyl, C₁₋₃ haloalkyl, C₃₋₆ cycloalkyl, or 4-6 membered heterocyclyl of R^{1a} and R^{1b} is independently optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R^{1a} and R^{1b} are taken together with the atoms to which they are attached to form 4-6 membered heterocyclyl independently optionally substituted by one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b};
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
R⁶ is hydrogen, C₁₋₃ alkyl, C₁₋₃ haloalkyl, C₃₋₆ cycloalkyl, or 4 to 6-membered heterocyclyl; wherein the C₁₋₃ alkyl, C₁₋₃ haloalkyl, C₃₋₆ cycloalkyl, or 4 to 6-membered heterocyclyl is optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
R⁷ is hydroxy, C₁₋₃ alkoxy, C₁₋₃ haloalkoxy, C₁₋₃ alkyl, or C₁₋₃ haloalkyl;
each Z^{A}, Z², Z^{2a}, Z³, Z^{3b}, Z⁴, and Z⁵, is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z², Z²ª, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}, or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, Ring B is a 5-membered heteroaryl optionally substituted with one R^{B}.

In some embodiments, provided is a compound represented by Formula IV:

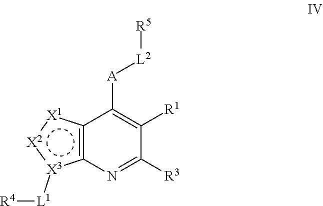

or a pharmaceutically acceptable salt, stereoisomer, mixture of stereoisomers, or solvate thereof, wherein:
each X¹, X², and X³ is independently N, CH, or CR^{B}, provided that at least one of X¹, X², and X³ is N, and the ring encompassing X¹, X², and X³ is aromatic;
A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroarylene of A is independently optionally substituted with one to five Z^{A};
each R^{B} is independently selected from halo, hydroxy, —NH₂—, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, C₁₋₃ heteroalkylene, or cyclopropylene; wherein the C₁₋₃ alkylene, C₂₋₃ alkenylene, C₂₋₃ alkynylene, or C₁₋₃ heteroalkylene, or cyclopropylene of L′ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
R¹ is cyano, —CH₂OH, —C(O)H, —C(O)NR^{1a}R^{1b}, —C(S) NR^{1a}R^{1b}, —S(O)₂R², —S(O)(NR⁶)R², or —P(O)R⁷R²;
R² is —NR^{1a}R^{1b}, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl of R² is optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R² and R⁶, or R² and R⁷, together with the atom to which they are attached, form a heterocyclyl optionally substituted with one to five Z²;
each R^{1a} and R^{1b} is independently hydrogen, C₁₋₃ alkyl, C₂₋₃ alkenyl, C₂₋₃ alkynyl, C₁₋₃ haloalkyl, C₃₋₆ cycloalkyl, or 4-6 membered heterocyclyl; wherein each C₁₋₃ alkyl, C₂₋₃ alkenyl, C₂₋₃ alkynyl, C₁₋₃ haloalkyl, C₃₋₆ cycloalkyl, or 4-6 membered heterocyclyl of R^{1a} and R^{1b} is independently optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R^{1a} and R^{1b} are taken together with the atoms to which they are attached to form 4-6 membered heterocyclyl independently optionally substituted by one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b};
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
R⁶ is hydrogen, C₁₋₃ alkyl, C₁₋₃ haloalkyl, C₃₋₆ cycloalkyl, or 4 to 6-membered heterocyclyl; wherein the C₁₋₃ alkyl, C₁₋₃ haloalkyl, C₃₋₆ cycloalkyl, or 4 to 6-membered heterocyclyl is optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
R⁷ is hydroxy, C₁₋₃ alkoxy, C₁₋₃ haloalkoxy, C₁₋₃ alkyl, or C₁₋₃ haloalkyl;
each Z^{A}, Z², Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z², Z^{2a}, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, provided is a compound represented by Formula IVA:

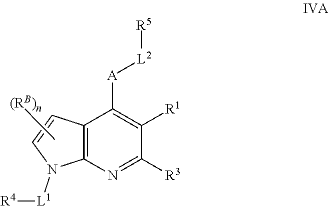

or a pharmaceutically acceptable salt, stereoisomer, mixture of stereoisomers, or solvate thereof, wherein L¹, L², A, R^{B}, R¹, R³, R⁴, and R⁵, are each independently as defined herein, and n is 0 or 1.

In some embodiments, provided is a compound represented by Formula IVB:

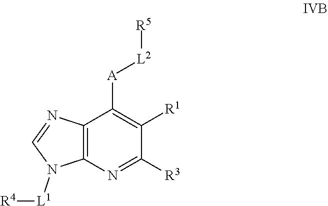

or a pharmaceutically acceptable salt, stereoisomer, mixture of stereoisomers, or solvate thereof, wherein L¹, L², A, R¹, R³, R⁴, and R⁵, are each independently as defined herein.

In some embodiments, provided is a compound represented by Formula IVB′:

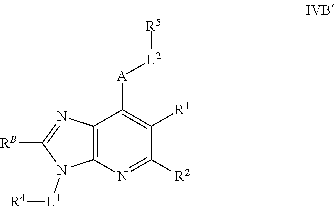

or a pharmaceutically acceptable salt, stereoisomer, mixture of stereoisomers, or solvate thereof, wherein L¹, L², A, R^{B}, R¹, R³, R⁴, and R⁵, are each independently as defined herein.

In some embodiments, provided is a compound represented by Formula IVC:

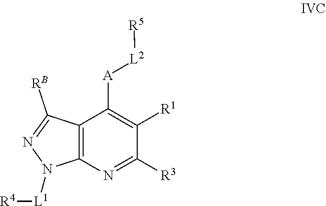

or a pharmaceutically acceptable salt, stereoisomer, mixture of stereoisomers, or solvate thereof, wherein L¹, L², A, R^{B}, R¹, R³, R⁴, and R⁵, are each independently as defined herein.

In some embodiments, provided is a compound represented by Formula IVA, IVB, IVB′, or IVC:

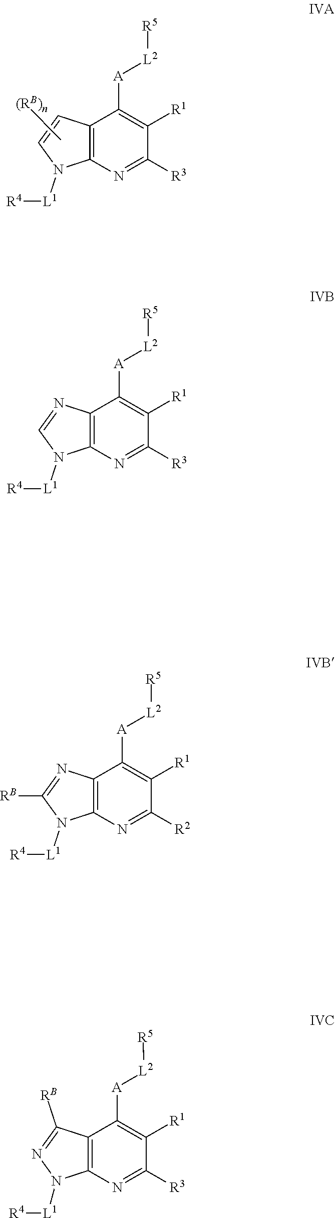

or a pharmaceutically acceptable salt, stereoisomer, mixture of stereoisomers, or solvate thereof, wherein:
n is 0 or 1;
A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroarylene of A is independently optionally substituted with one to five Z^{A};
R^{B} is selected from halo, hydroxy, —NH₂—, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, C₁₋₃ heteroalkylene, or cyclopropylene; wherein the C₁₋₃ alkylene, C₂₋₃ alkenylene, C₂₋₃ alkynylene, or C₁₋₃ heteroalkylene, or cyclopropylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
R¹ is cyano, —CH₂OH, —C(O)H, —C(O)NR^{1a}R^{1b}, —C(S)NR^{1a}R^{1b}, —S(O)₂R², —S(O)(NR⁶)R², or —P(O)R⁷R²;
R² is —NR^{1a}R^{1b}, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl of R² is optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R² and R⁶, or R² and R⁷, together with the atom to which they are attached, form a heterocyclyl optionally substituted with one to five Z²;
each R^{1a} and R^{1b} is independently hydrogen, C₁₋₃ alkyl, C₂₋₃ alkenyl, C₂₋₃ alkynyl, C₁₋₃ haloalkyl, C₃₋₆ cycloalkyl, or 4-6 membered heterocyclyl; wherein each C₁₋₃ alkyl, C₂₋₃ alkenyl, C₂₋₃ alkynyl, C₁₋₃ haloalkyl, C₃₋₆ cycloalkyl, or 4-6 membered heterocyclyl of R^{1a} and R^{1b} is independently optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R^{1a} and R^{1b} are taken together with the atoms to which they are attached to form 4-6 membered heterocyclyl independently optionally substituted by one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b};
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
R⁶ is hydrogen, C₁₋₃ alkyl, C₁₋₃ haloalkyl, C₃₋₆ cycloalkyl, or 4 to 6-membered heterocyclyl; wherein the C₁₋₃ alkyl, C₁₋₃ haloalkyl, C₃₋₆ cycloalkyl, or 4 to 6-membered heterocyclyl is optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
R⁷ is hydroxy, C₁₋₃ alkoxy, C₁₋₃ haloalkoxy, C₁₋₃ alkyl, or C₁₋₃ haloalkyl;
each Z^{A}, Z², Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z², Z^{2a}, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, Ring B is a C₅₋₆ cycloalkyl optionally substituted with one to three R^{B}.

In some embodiments, provided is a compound represented by Formula V:

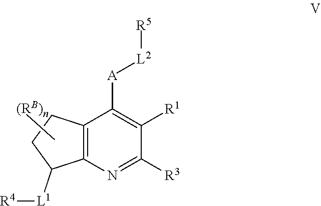

or a pharmaceutically acceptable salt, stereoisomer, mixture of stereoisomers, or solvate thereof, wherein:
n is 0 or 1;
A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroarylene of A is independently optionally substituted with one to five Z^{A};
R^{B} is selected from halo, hydroxy, —NH₂—, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, C₁₋₃ heteroalkylene, or cyclopropylene;
wherein the C₁₋₃ alkylene, C₂₋₃ alkenylene, C₂₋₃ alkynylene, or C₁₋₃ heteroalkylene, or cyclopropylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
R¹ is cyano, —CH₂OH, —C(O)H, —C(O)NR^{1a}R^{1b}, —C(S) NR^{1a}R^{1b}, —S(O)₂R², —S(O)(NR⁶)R², or —P(O)R⁷R²;
R² is —NR^{1a}R^{1b}, C₂₋₆ alkyl, C₂₋₆ alkonyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl of R² is optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R² and R⁶, or R² and R⁷, together with the atom to which they are attached, form a heterocyclyl optionally substituted with one to five Z²;
each R^{1a} and R^{1b} is independently hydrogen, C₁₋₃ alkyl, C₂₋₃ alkenyl, C₂₋₃ alkynyl, C₁₋₃ haloalkyl, C₃₋₆ cycloalkyl, or 4-6 membered heterocyclyl; wherein each C₁₋₃ alkyl, C₂₋₃ alkenyl, C₂₋₃ alkynyl, C₁₋₃ haloalkyl, C₃₋₆ cycloalkyl, or 4-6 membered heterocyclyl of R^{1a} and R^{1b} is independently optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
or R^{1a} and R^{1b} are taken together with the atoms to which they are attached to form 4-6 membered heterocyclyl independently optionally substituted by one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy;
each R^{2a} and R^{2b} is independently hydrogen, C₁₋₆ alkyl, C₂₋₆ alkonyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b},
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
R⁶ is hydrogen, C₁₋₃ alkyl, C₁₋₃ haloalkyl, C₃₋₆ cycloalkyl, or 4 to 6-membered heterocyclyl; wherein the C₁₋₃ alkyl, C₁₋₃ haloalkyl, C₃₋₆ cycloalkyl, or 4 to 6-membered heterocyclyl is optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
R⁷ is hydroxy, C₁₋₃ alkoxy, C₁₋₃ haloalkoxy, C₁₋₃ alkyl, or C₁₋₃ haloalkyl;
each Z^{A}, Z², Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z², Z^{2a}, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}, or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, each R^{B} is independently halo, hydroxy, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy. In some embodiments, R^{B} is halo, C₁₋₃ alkyl, or C₂₋₃ alkenyl; wherein the C₁₋₃ alkyl is optionally substituted with hydroxy. In some embodiments, R^{B} is C₁₋₃ alkyl optionally substituted with —NH₂, —NHC₁₋₃ alkyl, —N(C₁₋₃ alkyl)₂, hydroxy, or C₁₋₃ alkoxy.

In some embodiments, R^{B} is C₁₋₃ alkyl or C₂₋₃ alkenyl. In some embodiments, R^{B} is methyl, ethyl, ethenyl. In some embodiments, R^{B} is methyl. In some embodiments, R^{B} is ethyl. In some embodiments, R^{B} is ethenyl.

In some embodiments, A is bicyclic C₃₋₁₀ cycloalkylene, bicyclic or tricyclic heterocyclylene, monocyclic heteroarylene, or bicyclic heteroarylene; wherein the bicyclic C₃₋₁₀ cycloalkylene, bicyclic or tricyclic heterocyclylene, monocyclic heteroarylene, or bicyclic heteroarylene is independently optionally substituted with one to five Z^{A}.

In some embodiments, A is C₃₋₁₀ cycloalkylene, heterocyclylene, arylene, or heteroarylene; wherein the C₃₋₁₀ cycloalkylene, heterocyclylene, arylene, or heteroarylene of A is independently optionally substituted with one to five Z^{A}.

In some embodiments, A is bicyclic C₃₋₁₀ cycloalkylene, bicyclic heterocyclylene, monocyclic heteroarylene, or bicyclic heteroarylene.

In some embodiments, A is selected from:

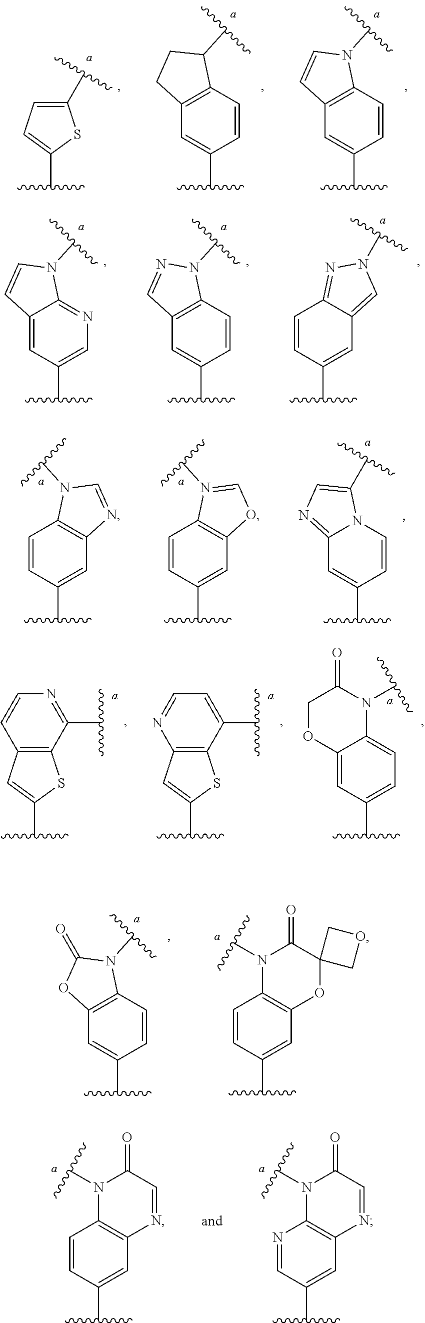

wherein each is optionally substituted with one to five Z^{A}; and bond a is bonded to L².

In some embodiments, A is selected from:

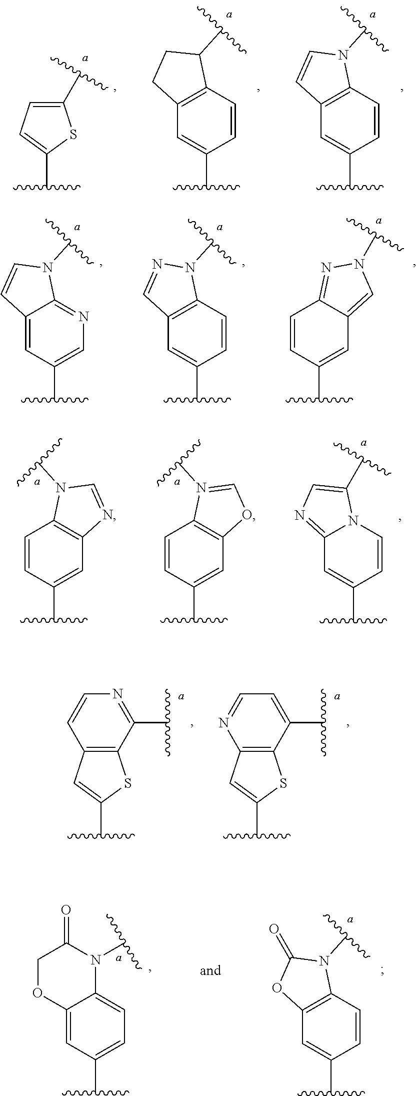

wherein each is optionally substituted with one to five Z^{A}; and bond a is bonded to L².

In some embodiments, A is selected from:

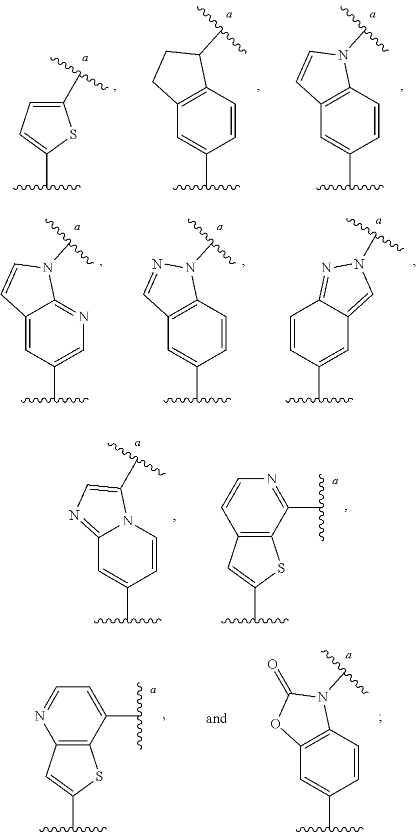

wherein each is optionally substituted with one to five Z^{A}, and wherein a is bonded to L².

In some embodiments, A is bicyclic heterocyclylene or bicyclic heteroarylene optionally substituted with one to five Z^{A}. In some embodiments, A is bicyclic heterocyclylene or bicyclic heteroarylene.

In some embodiments, A is bicyclic heterocyclylene containing an oxo.

In some embodiments, L² is a bond, —O—, —S—, —NR^{2a}—, —C(O)—, —C(O)O—, —OC(O)—, —OC(O)O—, —C(O)NR^{2a}—, —NR^{2a}C(O)—, —S(O)—, —S(O)₂—, C₁₋₆ alkylene, C₂₋₆ alkenylene, C₂₋₆ alkynylene, C₁₋₆ heteroalkylene, or C₃₋₆ cycloalkylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy.

In some embodiments, L² is a bond, —O—, —NR^{2a}—, —C(O)—, —C(O)O—, —OC(O)—, —C(O)NR^{2a}—, —S(O)—, —S(O)₂—, C₁₋₆ alkylene, C₁₋₆ heteroalkylene optionally substituted with oxo, or C₃₋₆ cycloalkylene.

In some embodiments, L² is a bond, —O—, —NR^{2a}—, —C(O)—, —C(O)O—, —OC(O)—, —C(O)NR^{2a}—, —S(O)—, —S(O)₂—, C₁₋₆ alkylene, C₁₋₆ heteroalkylene, or C₃₋₆ cycloalkylene.

In some embodiments, L² is —NR^{2a}—, —C(O)NR^{2a}—, C₁₋₆ alkylene, C₁₋₆ heteroalkylene, or C₃₋₆ cycloalkylene.

In some embodiments, L² is —NH₂—, —CH₂—, —CH₂CH₂—, —CH(CH₃)—, —CH(CH₂CH₃)—, —CH(CH(CH₃)₂)—, —CH(CH₂OH)—, —C(O)NH₂—, —O—CH₂—, —NH—CH₂—, or cyclopropylene.

In some embodiments, L² is —NH₂—, —CH₂—, —CH₂CH₂—, —CH(CH₃)—, —CH(CH₂CH₃)—, —C(O) NH₂—, —O—CH₂—, —NH—CH₂—, or cyclopropylene.

In some embodiments, L² is C₁₋₆ alkylene. In some embodiments, L² is branched C₁₋₆ alkylene. In some embodiments, L² is —CH(CH₃)—, —CH(CH₂CH₃)—, —CH(CH₂CH₂CH₃)—, —C(CH₃)₂—, —C(CH₂CH₃)₂—, or —C(CH₃)(CH₂CH₃)—.

In some embodiments, A is bicyclic C₃₋₁₀ cycloalkylene, bicyclic heterocyclylene, monocyclic heteroarylene, or bicyclic heteroarylene and L² is C₁₋₆ alkylene. In some embodiments, A is bicyclic C₃₋₁₀ cycloalkylene, bicyclic heterocyclylene, monocyclic heteroarylene, or bicyclic heteroarylene and L² is —CH₂—, —CH₂CH₂—, —CH(CH₃)—, or —CH(CH₂CH₃)—.

In some embodiments, A is bicyclic C₃₋₁₀ cycloalkylene, bicyclic heterocyclylene, monocyclic heteroarylene, or bicyclic heteroarylene and L² is branched C₁₋₆ alkylene. In some embodiments, A is bicyclic heterocyclylene or bicyclic heteroarylene and L² is branched C₁₋₆ alkylene. In some embodiments, A is bicyclic heterocyclylene or bicyclic heteroarylene and L² is —CH(CH₃)—, —CH(CH₂CH₃)—, —CH(CH₂CH₂CH₃)—, —C(CH₃)₂—, —C(CH₂CH₃)₂—, or —C(CH₃)(CH₂CH₃)—. In some embodiments, A is bicyclic heterocyclylene or bicyclic heteroarylene and L² is —CH(CH₃)— or —CH(CH₂CH₃)—.

In some embodiments, the moiety

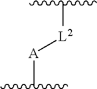

is selected from:

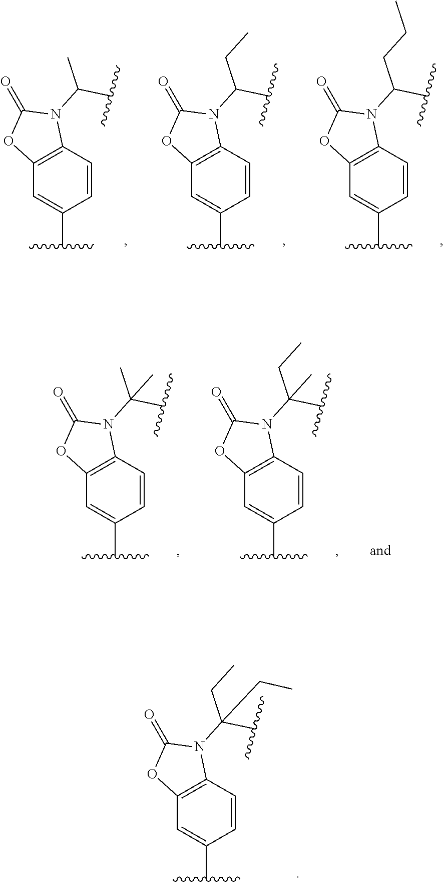 [SMILES: *c1ccc2c(c1)oc(=O)n2C(*)C ; *c1ccc2c(c1)oc(=O)n2C(*)CC ; *c1ccc2c(c1)oc(=O)n2C(*)CCC ; *c1ccc2c(c1)oc(=O)n2C(*)(C)C ; *c1ccc2c(c1)oc(=O)n2C(*)(C)CC ; *c1ccc2c(c1)oc(=O)n2C(*)(CC)CC]

In some embodiments, the moiety

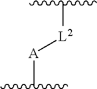

is selected from:

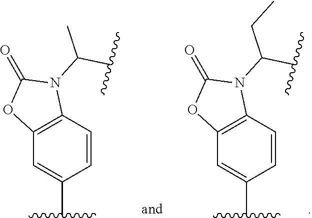 [SMILES: *c1ccc2c(c1)oc(=O)n2C(*)C ; *c1ccc2c(c1)oc(=O)n2C(*)CC]

In some embodiments, provided is a compound of Formula VI:

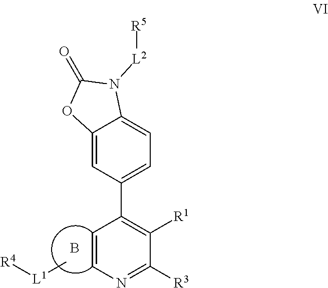

or a pharmaceutically acceptable salt, stereoisomer, mixture of stereoisomers, or solvate thereof, wherein Ring B, L¹, L², R¹, R³, R⁴, and R⁵ are each independently as defined herein. In some embodiments, L² is branched C₁₋₆ alkylene. In some embodiments, L² is —CH(CH₃)—, —CH(CH₂CH₃)—, —CH(CH₂CH₂CH₃)—, —C(CH₃)₂—, —C(CH₂CH₃)₂—, or —C(CH₃)(CH₂CH₃)—.

In some embodiments, provided is a compound of Formula VIA:

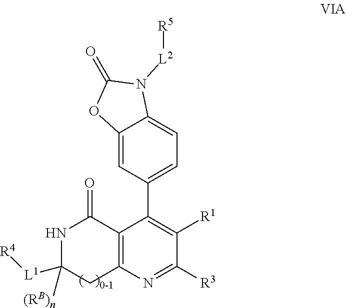

or a pharmaceutically acceptable salt, stereoisomer, mixture of stereoisomers, or solvate thereof, wherein R^{B}, L¹, L², R¹, R³, R⁴, and R⁵ are each independently as defined herein, and n is 0 or 1. In some embodiments, L² is branched C₁₋₆ alkylene. In some embodiments, L² is —CH(CH₃)—, —CH(CH₂CH₃)—, —CH(CH₂CH₂CH₃)—, —C(CH₃)₂—, —C(CH₂CH₃)₂—, or —C(CH₃)(CH₂CH₃)—.

In some embodiments, provided is a compound of Formula VII:

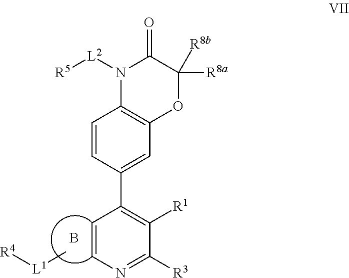

or a pharmaceutically acceptable salt, stereoisomer, mixture of stereoisomers, or solvate thereof, wherein Ring B, L¹, L², R¹, R³, R⁴, and R⁵ are each independently as defined herein, and R^{8a} and R^{8b} are each independently hydrogen, halo, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{1a} and R^{1b} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{8a} and R^{8b} are taken together with the nitrogen atom to which they are attached to form a heterocyclyl optionally substituted with one to five substituents independently selected from halo, hydroxy, cyano, C₁₋₃ alkyl, C₁₋₃ haloalkyl, C₁₋₃ alkoxy, and C₁₋₃ haloalkoxy.

In some embodiments, provided is a compound of Formula VIII:

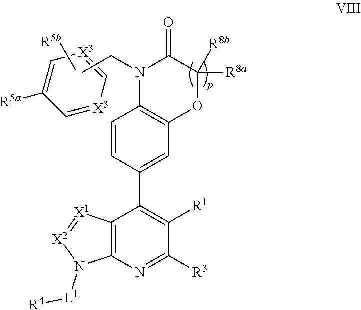

or a pharmaceutically acceptable salt, stereoisomer, mixture of stereoisomers, or solvate thereof, wherein L¹, R¹, R³, and R⁴ are each independently as defined herein,
X¹ and X² are each independently N or CR^{B};
each X³ is independently N or CR^{5c};
R^{B} is selected from halo, hydroxy, —NH₂—, cyano, C₁₋₃ alkyl, C₂₋₃ alkenyl, C₂₋₃ alkynyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
each R^{5a}, R^{5b}, and R^{5c} are independently hydrogen, halo, hydroxy, cyano, C₁₋₃ alkyl, C₁₋₃ haloalkyl, C₁₋₃ alkoxy, or C₁₋₃ haloalkoxy;
p is 0 or 1;
R^{8a} and R^{8b} are each independently hydrogen, halo, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{1a} and R^{1b} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{8a} and R^{8b} are taken together with the nitrogen atom to which they are attached to form a heterocyclyl optionally substituted with one to five substituents independently selected from halo, hydroxy, cyano, C₁₋₃ alkyl, C₁₋₃ haloalkyl, C₁₋₃ alkoxy, and C₁₋₃ haloalkoxy.

In some embodiments, R^{8a} and R^{8b} are each independently hydrogen, halo, or C₁₋₆ alkyl.

In some embodiments, R⁵ is C₁₋₆ haloalkyl, C₃₋₁₀ cycloalkyl, heterocyclyl, aryl, or heteroaryl; wherein the C₃₋₁₀ cycloalkyl, heterocyclyl, aryl, or heteroaryl of R⁵ is independently optionally substituted with one to five Z⁵.

In some embodiments, R⁵ is C₃₋₁₀ cycloalkyl, heterocyclyl, aryl, or heteroaryl; wherein the C₃₋₁₀ cycloalkyl, heterocyclyl, aryl, or heteroaryl of R⁵ is independently optionally substituted with one to five Z⁵.

In some embodiments, R⁵ is C₃₋₁₀ cycloalkyl, heterocyclyl, aryl, or heteroaryl; wherein the C₃₋₁₀ cycloalkyl, heterocyclyl, aryl, or heteroaryl of R⁵ is independently optionally substituted with one to five halo, cyano, or —O—C₁₋₆ alkyl.

In some embodiments, R⁵ is phenyl, pyridyl, 2,3-dihydro-1H-indenyl, or 6,7-dihydro-5H-cyclopenta[b]pyridine; wherein each is independently optionally substituted with one to three halo, cyano, or —O—C₁₋₆ alkyl.

In some embodiments, R⁵ is selected from:

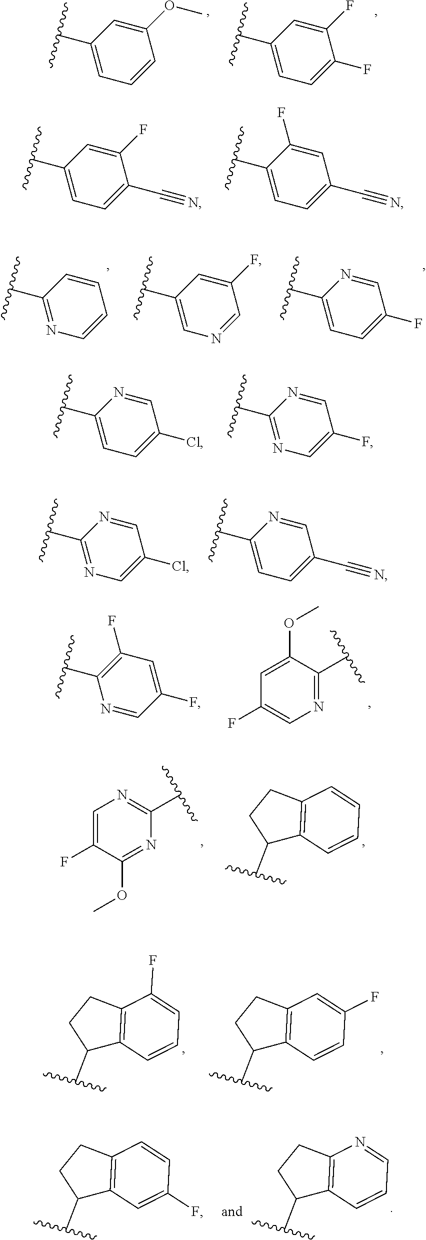

In some embodiments, R⁵ is selected from:

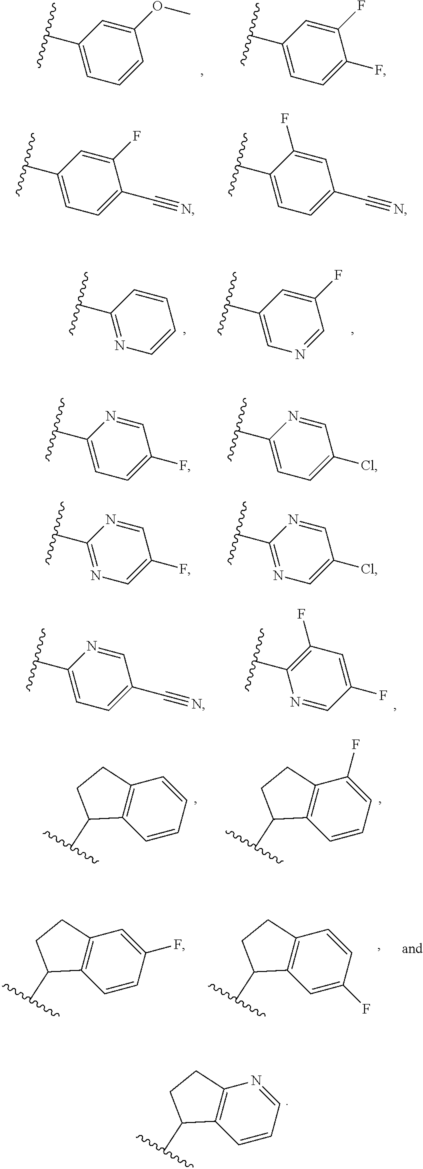

In some embodiments, R⁵ is selected from:

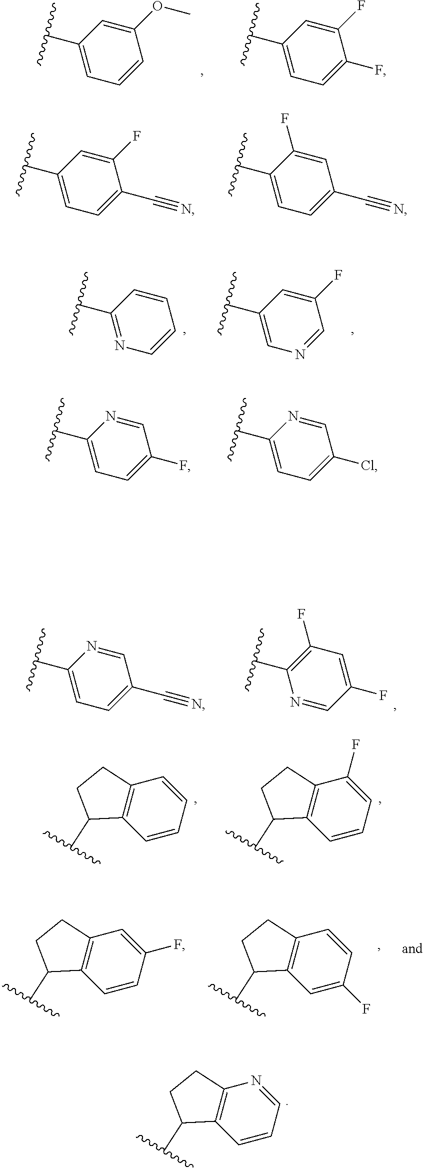

In some embodiments, L¹ is cyclopropylene, C₁₋₃ alkylene optionally substituted with one to five halo, or C₁₋₆ heteroalkylene substituted with oxo.

In some embodiments, L¹ is cyclopropylene or C₁₋₃ alkylene optionally substituted with one to five halo.

In some embodiments, L¹ is cyclopropylene.

In some embodiments, L¹ is

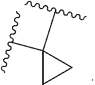 [SMILES: *C1(*)CC1]

In some embodiments, L¹ is C₁₋₃ alkylene, C₂₋₃ alkenylene, C₂₋₃ alkynylene, or C₁₋₃ heteroalkylene; wherein the C₁₋₃ alkylene, C₂₋₃ alkenylene, C₂₋₃ alkynylene, or C₁₋₃ heteroalkylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy.

In some embodiments, L¹ is C₁₋₃ alkylene or C₁₋₃ heteroalkylene; wherein L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy.

In some embodiments, L¹ is C₁₋₃ alkylene, C₂₋₃ alkenylene, C₂₋₃ alkynylene, or C₁₋₃ heteroalkylene.

In some embodiments, L¹ is C₁₋₃ alkylene. In some embodiments, L¹ is —CH₂—, —CH(CH₃)—, —CH₂CH₂—, —CH₂CH₂CH₂—, —CH(CH₃)CH₂—, or —CH₂CH(CH₃)—. In some embodiments, L¹ is —CH₂—, —CH₂CH₂—, or —CH(CH₃)—.

In some embodiments, R⁴ is C₁₋₃ alkyl, C₃₋₁₀ cycloalkyl, phenyl, heterocyclyl, or heteroaryl; wherein the C₁₋₃ alkyl, C₃₋₁₀ cycloalkyl, phenyl, heterocyclyl, or heteroaryl of R⁴ is independently optionally substituted with one to five Z⁴.

In some embodiments, R⁴ is C₁₋₃ alkyl,

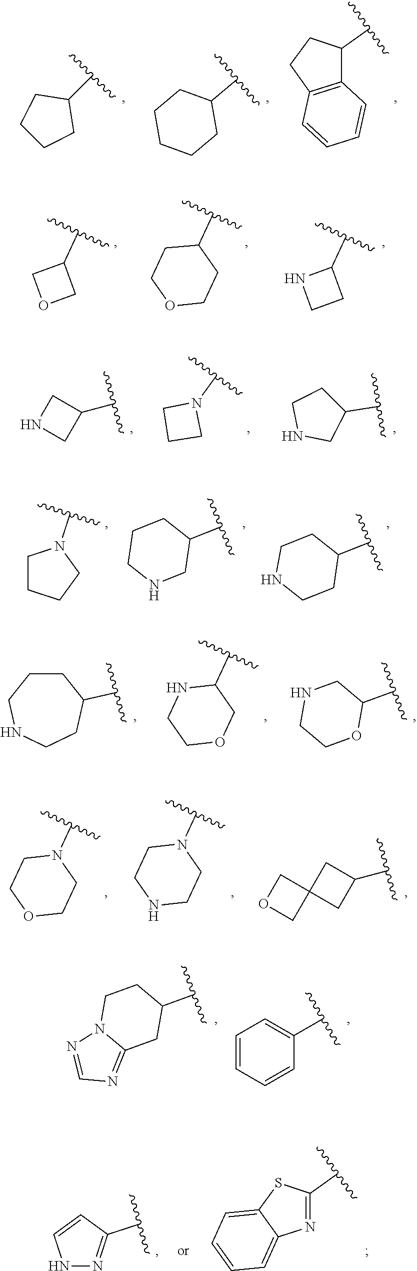

wherein each is optionally substituted with one to five Z⁴.

In some embodiments, R⁴ is C₃₋₁₀ cycloalkyl, aryl, or heteroaryl; wherein the C₃₋₁₀ cycloalkyl, aryl, or heteroaryl of R⁴ is independently optionally substituted with one to five Z⁴.

In some embodiments, R⁴ is

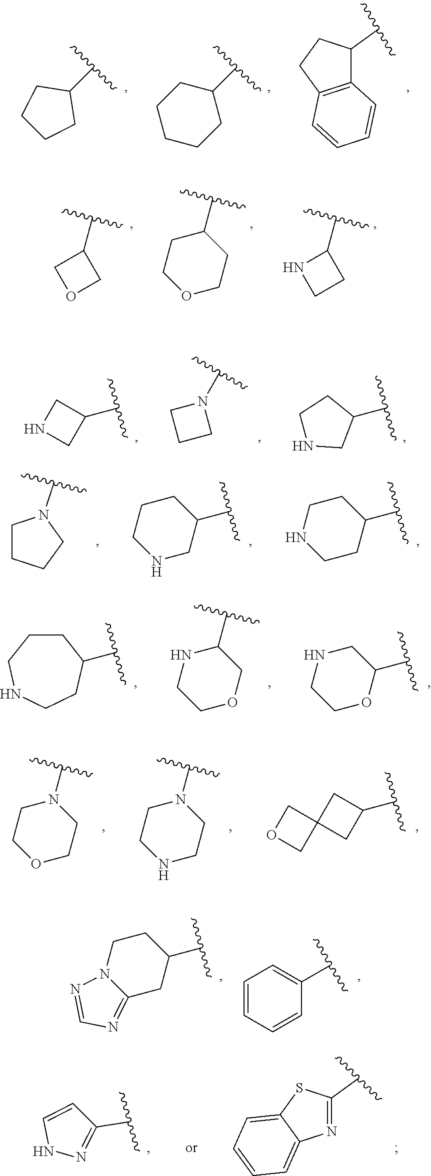

wherein each is optionally substituted with one to five Z⁴.

In some embodiments, each Z⁴ is independently halo, -L-C₁₋₆ alkyl, -L-C₁₋₆ haloalkyl.

In some embodiments, R⁴ is C₃₋₁₀ cycloalkyl, aryl, or heteroaryl; wherein the C₃₋₁₀ cycloalkyl, aryl, or heteroaryl of R⁴ is independently optionally substituted with one to three halo.

In some embodiments, R⁴ is phenyl, 2,3-dihydro-1H-indenyl, or benzothiazolyl; wherein each is optionally substituted with one to three halo.

In some embodiments, R⁴ is 4-fluorophenyl, 2,3-dihydro-1H-indenyl, or benzothiazolyl.

In some embodiments, each Z⁴ and Z⁵ is independently halo, cyano, C₁₋₆ alkyl, C₁₋₆ haloalkyl, or -L-C₁₋₆ alkyl. In some embodiments, L is —O—. In some embodiments, each Z⁴ and Z⁵ is independently halo, cyano, or —O—C₁₋₆ alkyl. In some embodiments, each Z⁴ and Z⁵ is independently fluoro, cyano, or methoxy.

In some embodiments, provided is compound selected from Table 1, or a pharmaceutically acceptable salt, stereoisomer, mixture of stereoisomers, or solvate thereof:

TABLE 1

| Compound No. | Structure |
|---|---|
| 101 | 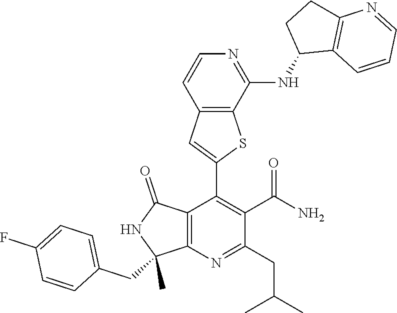 [SMILES: CC(C)Cc1nc2c(c(-c3cc4ccnc(N[C@@H]5CCc6ncccc65)c4s3)c1C(N)=O)C(=O)N[C@@]2(C)Cc1ccc(F)cc1] |
| 102 | 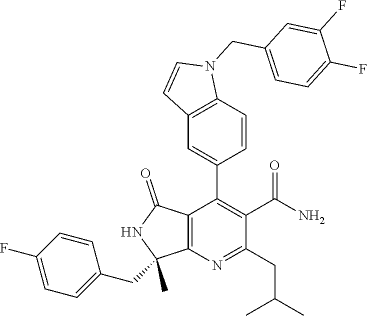 [SMILES: CC(C)Cc1nc2c(c(-c3ccc4c(ccn4Cc4ccc(F)c(F)c4)c3)c1C(N)=O)C(=O)N[C@@]2(C)Cc1ccc(F)cc1] |
| 103 | 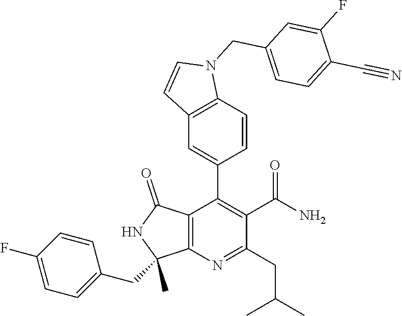 [SMILES: CC(C)Cc1nc2c(c(-c3ccc4c(ccn4Cc4ccc(C#N)c(F)c4)c3)c1C(N)=O)C(=O)N[C@@]2(C)Cc1ccc(F)cc1] |
| 104 | 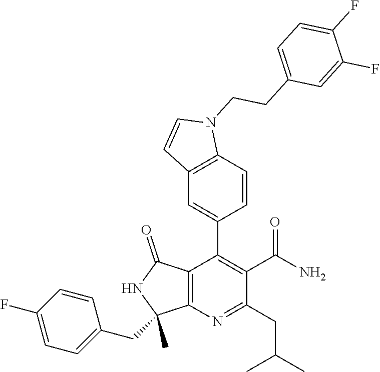 [SMILES: CC(C)Cc1nc2c(c(-c3ccc4c(ccn4CCc4ccc(F)c(F)c4)c3)c1C(N)=O)C(=O)N[C@@]2(C)Cc1ccc(F)cc1] |
| 105 | 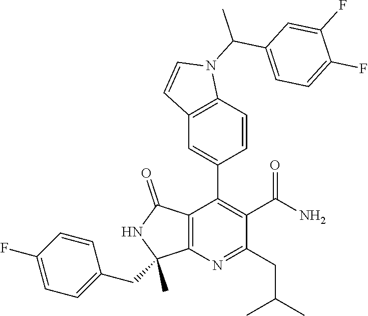 [SMILES: CC(C)Cc1nc2c(c(-c3ccc4c(ccn4C(C)c4ccc(F)c(F)c4)c3)c1C(N)=O)C(=O)N[C@@]2(C)Cc1ccc(F)cc1] |
| 106 | 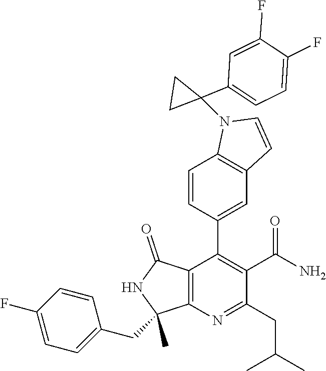 [SMILES: CC(C)Cc1nc2c(c(-c3ccc4c(ccn4C4(c5ccc(F)c(F)c5)CC4)c3)c1C(N)=O)C(=O)N[C@@]2(C)Cc1ccc(F)cc1] |
| 107 | 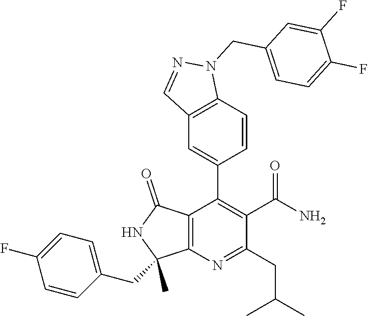 [SMILES: CC(C)Cc1nc2c(c(-c3ccc4c(cnn4Cc4ccc(F)c(F)c4)c3)c1C(N)=O)C(=O)N[C@@]2(C)Cc1ccc(F)cc1] |
| 108 | 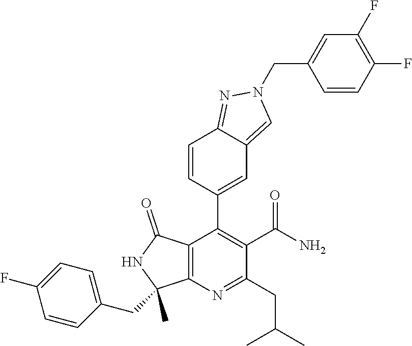 [SMILES: CC(C)Cc1nc2c(c(-c3ccc4nn(Cc5ccc(F)c(F)c5)cc4c3)c1C(N)=O)C(=O)N[C@@]2(C)Cc1ccc(F)cc1] |
| 109 | 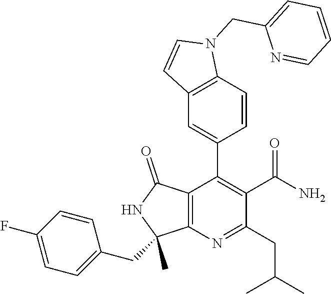 [SMILES: CC(C)Cc1nc2c(c(-c3ccc4c(ccn4Cc4ccccn4)c3)c1C(N)=O)C(=O)N[C@@]2(C)Cc1ccc(F)cc1] |
| 110 | 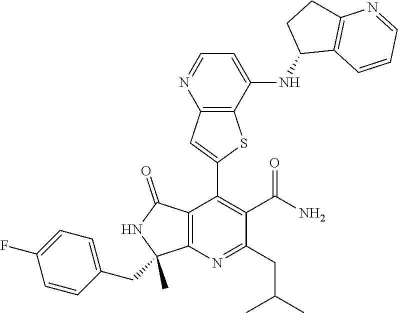 [SMILES: CC(C)Cc1nc2c(c(-c3cc4nccc(N[C@@H]5CCc6ncccc65)c4s3)c1C(N)=O)C(=O)N[C@@]2(C)Cc1ccc(F)cc1] |
| 111 | 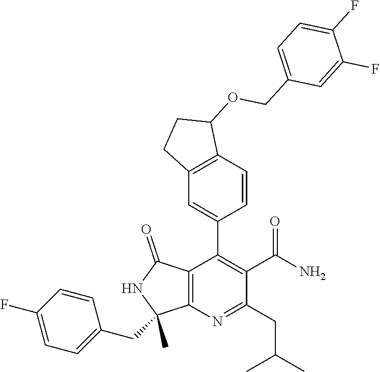 [SMILES: CC(C)Cc1nc2c(c(-c3ccc4c(c3)CCC4OCc3ccc(F)c(F)c3)c1C(N)=O)C(=O)N[C@@]2(C)Cc1ccc(F)cc1] |
| 112 | 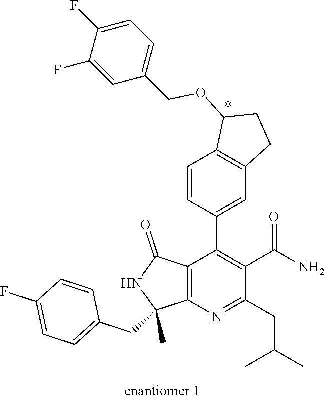 [SMILES: CC(C)Cc1nc2c(c(-c3ccc4c(c3)CCC4OCc3ccc(F)c(F)c3)c1C(N)=O)C(=O)N[C@@]2(C)Cc1ccc(F)cc1] |
| 113 | 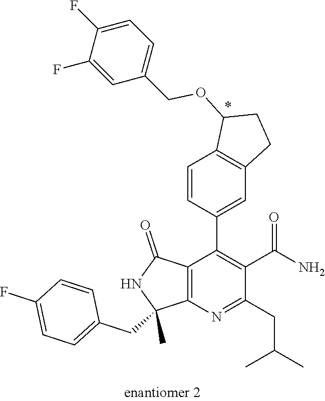 [SMILES: CC(C)Cc1nc2c(c(-c3ccc4c(c3)CCC4OCc3ccc(F)c(F)c3)c1C(N)=O)C(=O)N[C@@]2(C)Cc1ccc(F)cc1] |
| 114 | 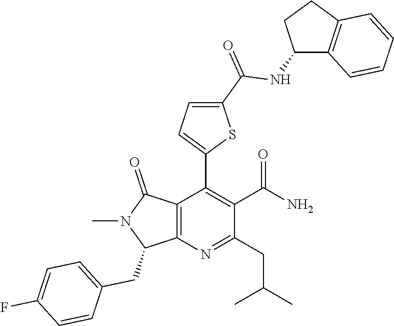 [SMILES: CC(C)Cc1nc2c(c(-c3ccc(C(=O)N[C@@H]4CCc5ccccc54)s3)c1C(N)=O)C(=O)N(C)[C@H]2Cc1ccc(F)cc1] |
| 115 | 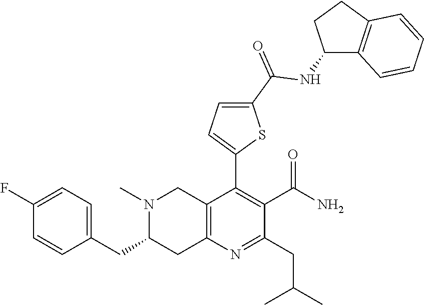 [SMILES: CC(C)Cc1nc2c(c(-c3ccc(C(=O)N[C@@H]4CCc5ccccc54)s3)c1C(N)=O)CN(C)[C@@H](Cc1ccc(F)cc1)C2] |
| 116 | 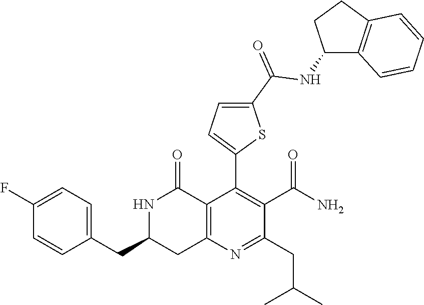 [SMILES: CC(C)Cc1nc2c(c(-c3ccc(C(=O)N[C@@H]4CCc5ccccc54)s3)c1C(N)=O)C(=O)N[C@H](Cc1ccc(F)cc1)C2] |
| 117 | 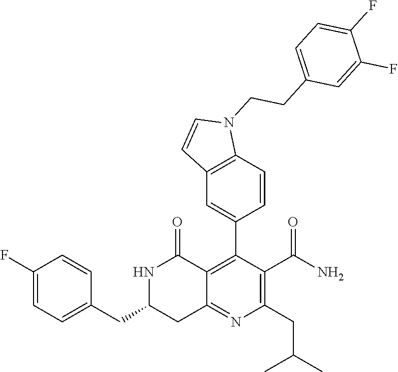 [SMILES: CC(C)Cc1nc2c(c(-c3ccc4c(ccn4CCc4ccc(F)c(F)c4)c3)c1C(N)=O)C(=O)N[C@@H](Cc1ccc(F)cc1)C2] |
| 118 | 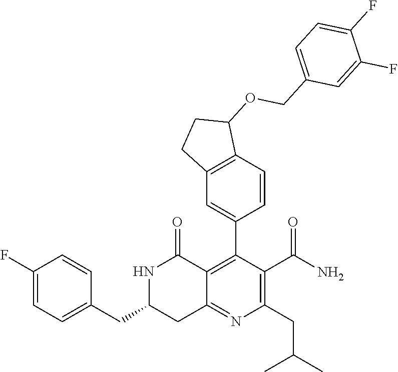 [SMILES: CC(C)Cc1nc2c(c(-c3ccc4c(c3)CCC4OCc3ccc(F)c(F)c3)c1C(N)=O)C(=O)N[C@@H](Cc1ccc(F)cc1)C2] |
| 119 | 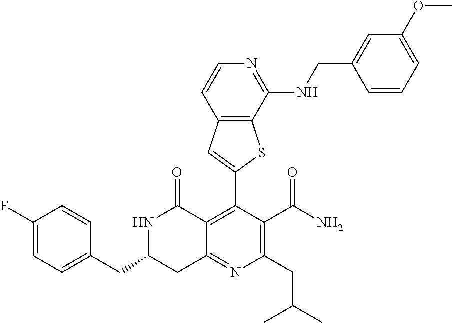 [SMILES: COc1cccc(CNc2nccc3cc(-c4c(C(N)=O)c(CC(C)C)nc5c4C(=O)N[C@@H](Cc4ccc(F)cc4)C5)sc23)c1] |
| 120 | 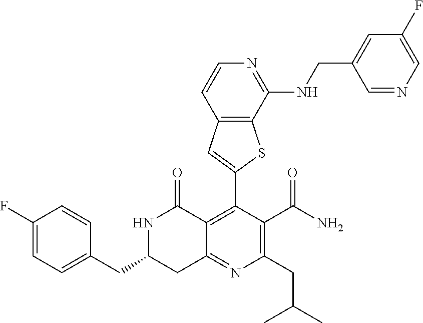 [SMILES: CC(C)Cc1nc2c(c(-c3cc4ccnc(NCc5cncc(F)c5)c4s3)c1C(N)=O)C(=O)N[C@@H](Cc1ccc(F)cc1)C2] |
| 121 | 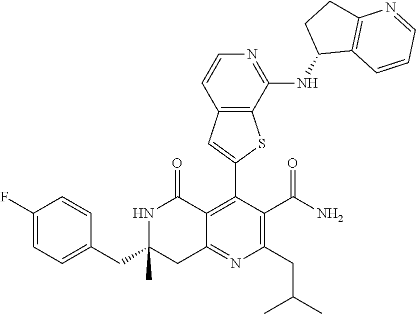 [SMILES: CC(C)Cc1nc2c(c(-c3cc4ccnc(N[C@@H]5CCc6ncccc65)c4s3)c1C(N)=O)C(=O)N[C@@](C)(Cc1ccc(F)cc1)C2] |
| 122 | 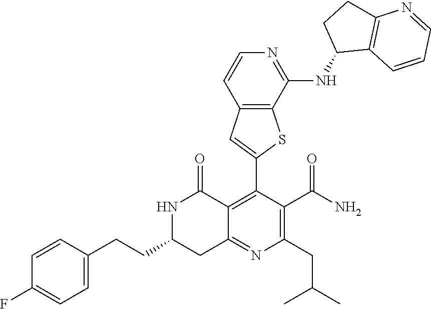 [SMILES: CC(C)Cc1nc2c(c(-c3cc4ccnc(N[C@@H]5CCc6ncccc65)c4s3)c1C(N)=O)C(=O)N[C@@H](CCc1ccc(F)cc1)C2] |
| 123 | 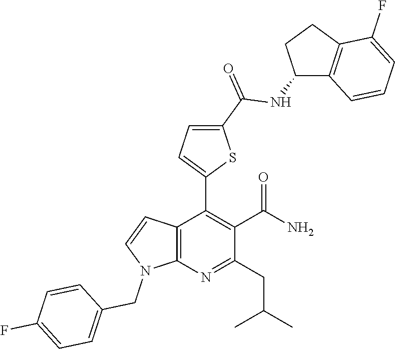 [SMILES: CC(C)Cc1nc2c(ccn2Cc2ccc(F)cc2)c(-c2ccc(C(=O)N[C@@H]3CCc4c(F)cccc43)s2)c1C(N)=O] |
| 124 | 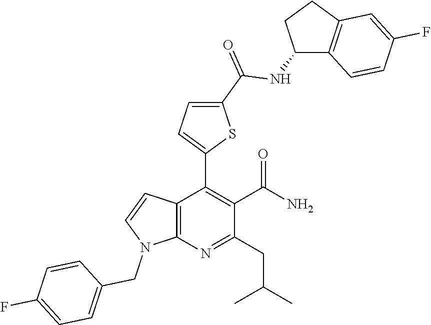 [SMILES: CC(C)Cc1nc2c(ccn2Cc2ccc(F)cc2)c(-c2ccc(C(=O)N[C@@H]3CCc4cc(F)ccc43)s2)c1C(N)=O] |
| 125 | 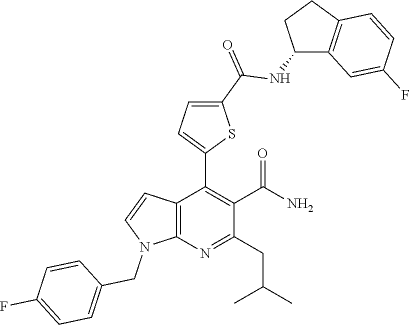 [SMILES: CC(C)Cc1nc2c(ccn2Cc2ccc(F)cc2)c(-c2ccc(C(=O)N[C@@H]3CCc4ccc(F)cc43)s2)c1C(N)=O] |
| 126 | 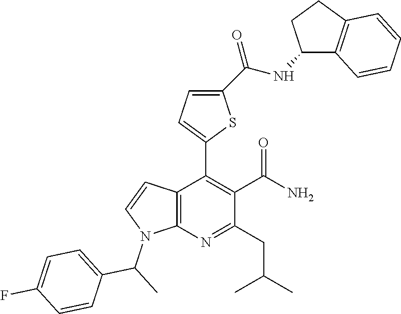 [SMILES: CC(C)Cc1nc2c(ccn2C(C)c2ccc(F)cc2)c(-c2ccc(C(=O)N[C@@H]3CCc4ccccc43)s2)c1C(N)=O] |
| 127 | 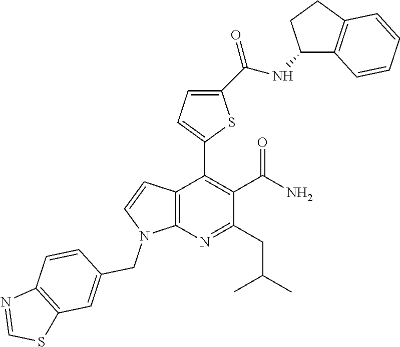 [SMILES: CC(C)Cc1nc2c(ccn2Cc2ccc3ncsc3c2)c(-c2ccc(C(=O)N[C@@H]3CCc4ccccc43)s2)c1C(N)=O] |
| 128 | 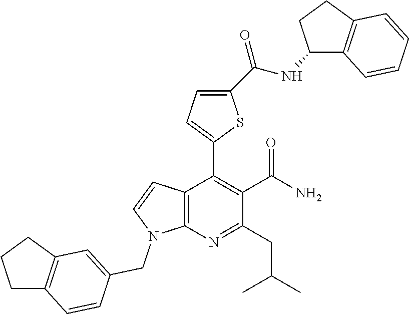 [SMILES: CC(C)Cc1nc2c(ccn2Cc2ccc3c(c2)CCC3)c(-c2ccc(C(=O)N[C@@H]3CCc4ccccc43)s2)c1C(N)=O] |
| 129 | 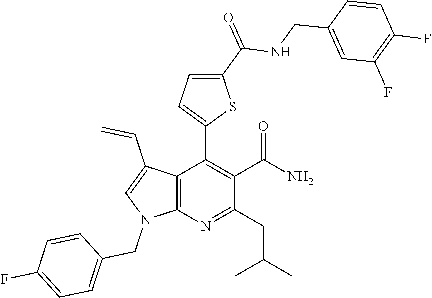 [SMILES: C=Cc1cn(Cc2ccc(F)cc2)c2nc(CC(C)C)c(C(N)=O)c(-c3ccc(C(=O)NCc4ccc(F)c(F)c4)s3)c12] |
| 130 | 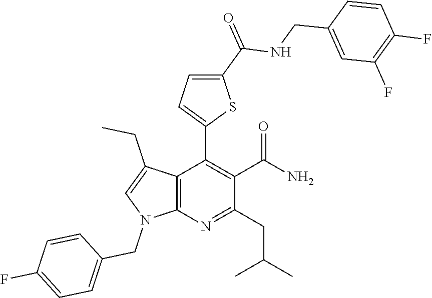 [SMILES: CCc1cn(Cc2ccc(F)cc2)c2nc(CC(C)C)c(C(N)=O)c(-c3ccc(C(=O)NCc4ccc(F)c(F)c4)s3)c12] |
| 131 | 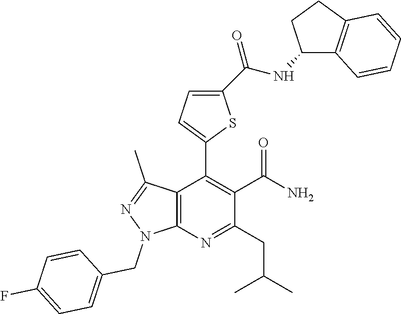 [SMILES: Cc1nn(Cc2ccc(F)cc2)c2nc(CC(C)C)c(C(N)=O)c(-c3ccc(C(=O)N[C@@H]4CCc5ccccc54)s3)c12] |
| 132 | 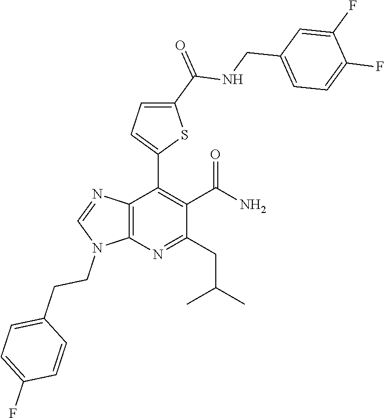 [SMILES: CC(C)Cc1nc2c(ncn2CCc2ccc(F)cc2)c(-c2ccc(C(=O)NCc3ccc(F)c(F)c3)s2)c1C(N)=O] |
| 133 | 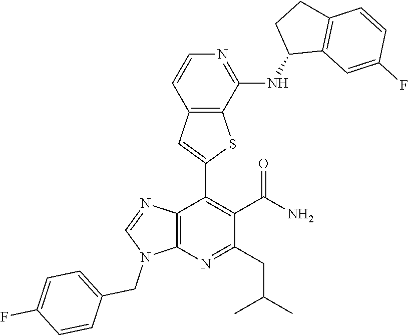 [SMILES: CC(C)Cc1nc2c(ncn2Cc2ccc(F)cc2)c(-c2cc3ccnc(N[C@@H]4CCc5ccc(F)cc54)c3s2)c1C(N)=O] |
| 134 | 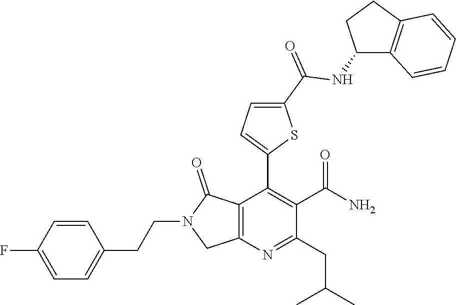 [SMILES: CC(C)Cc1nc2c(c(-c3ccc(C(=O)N[C@@H]4CCc5ccccc54)s3)c1C(N)=O)C(=O)N(CCc1ccc(F)cc1)C2] |
| 135 | 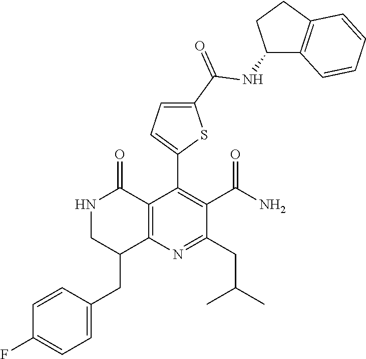 [SMILES: CC(C)Cc1nc2c(c(-c3ccc(C(=O)N[C@@H]4CCc5ccccc54)s3)c1C(N)=O)C(=O)NCC2Cc1ccc(F)cc1] |
| 136 | 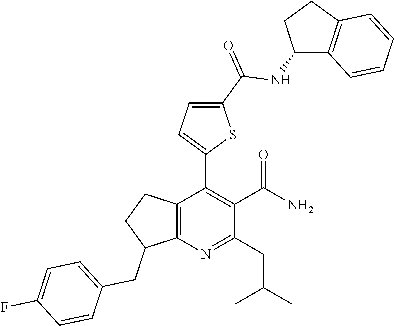 [SMILES: CC(C)Cc1nc2c(c(-c3ccc(C(=O)N[C@@H]4CCc5ccccc54)s3)c1C(N)=O)CCC2Cc1ccc(F)cc1] |
| 141 | 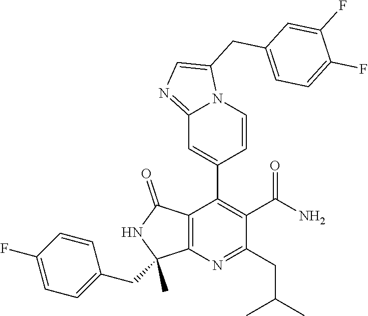 [SMILES: CC(C)Cc1nc2c(c(-c3ccn4c(Cc5ccc(F)c(F)c5)cnc4c3)c1C(N)=O)C(=O)N[C@@]2(C)Cc1ccc(F)cc1] |
| 142 | 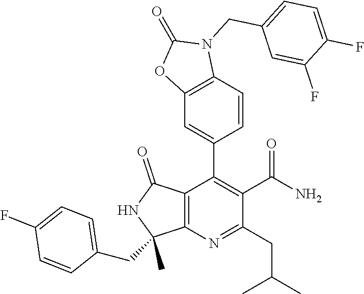 [SMILES: CC(C)Cc1nc2c(c(-c3ccc4c(c3)oc(=O)n4Cc3ccc(F)c(F)c3)c1C(N)=O)C(=O)N[C@@]2(C)Cc1ccc(F)cc1] |
| 143 | 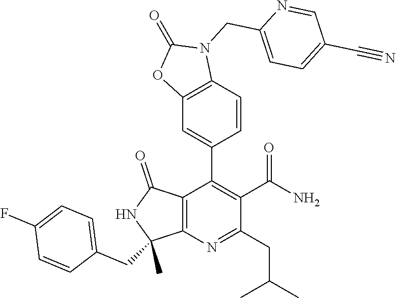 [SMILES: CC(C)Cc1nc2c(c(-c3ccc4c(c3)oc(=O)n4Cc3ccc(C#N)cn3)c1C(N)=O)C(=O)N[C@@]2(C)Cc1ccc(F)cc1] |
| 144 | 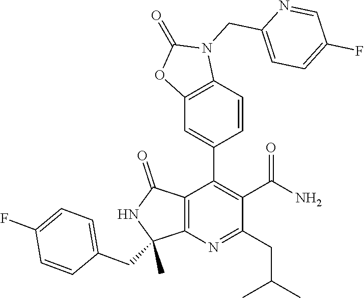 [SMILES: CC(C)Cc1nc2c(c(-c3ccc4c(c3)oc(=O)n4Cc3ccc(F)cn3)c1C(N)=O)C(=O)N[C@@]2(C)Cc1ccc(F)cc1] |
| 145 | 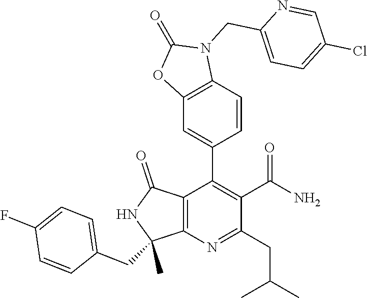 [SMILES: CC(C)Cc1nc2c(c(-c3ccc4c(c3)oc(=O)n4Cc3ccc(Cl)cn3)c1C(N)=O)C(=O)N[C@@]2(C)Cc1ccc(F)cc1] |
| 146 | 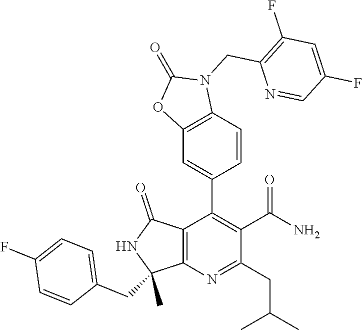 [SMILES: CC(C)Cc1nc2c(c(-c3ccc4c(c3)oc(=O)n4Cc3ncc(F)cc3F)c1C(N)=O)C(=O)N[C@@]2(C)Cc1ccc(F)cc1] |
| 147 | 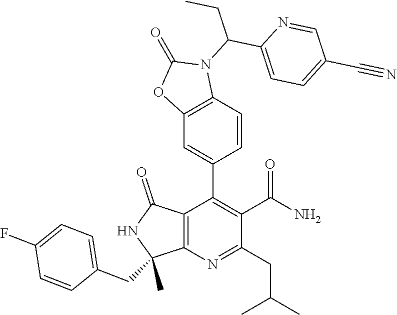 [SMILES: CCC(c1ccc(C#N)cn1)n1c(=O)oc2cc(-c3c(C(N)=O)c(CC(C)C)nc4c3C(=O)N[C@@]4(C)Cc3ccc(F)cc3)ccc21] |
| 148 | 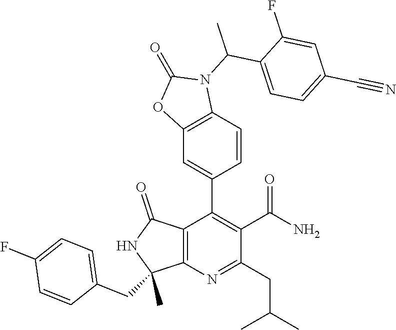 [SMILES: CC(C)Cc1nc2c(c(-c3ccc4c(c3)oc(=O)n4C(C)c3ccc(C#N)cc3F)c1C(N)=O)C(=O)N[C@@]2(C)Cc1ccc(F)cc1] |
| 149 | 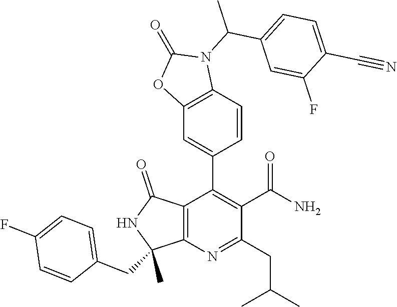 [SMILES: CC(C)Cc1nc2c(c(-c3ccc4c(c3)oc(=O)n4C(C)c3ccc(C#N)c(F)c3)c1C(N)=O)C(=O)N[C@@]2(C)Cc1ccc(F)cc1] |
| 150 | 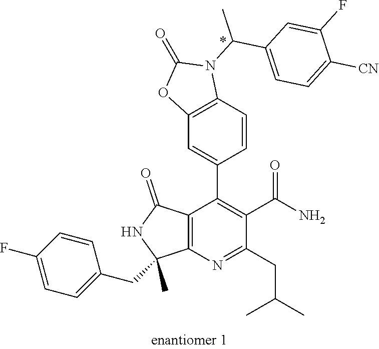 [SMILES: CC(C)Cc1nc2c(c(-c3ccc4c(c3)oc(=O)n4C(C)c3ccc(C#N)c(F)c3)c1C(N)=O)C(=O)N[C@@]2(C)Cc1ccc(F)cc1] |
| 151 | 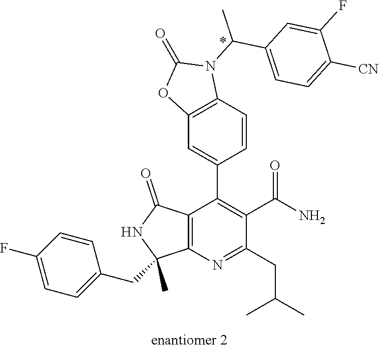 [SMILES: CC(C)Cc1nc2c(c(-c3ccc4c(c3)oc(=O)n4C(C)c3ccc(C#N)c(F)c3)c1C(N)=O)C(=O)N[C@@]2(C)Cc1ccc(F)cc1] |
| 152 | 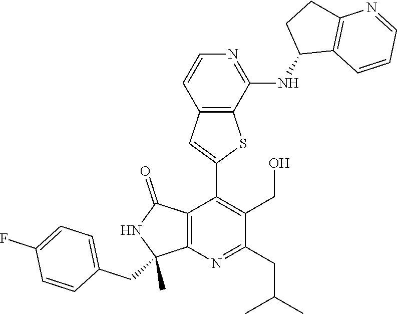 [SMILES: CC(C)Cc1nc2c(c(-c3cc4ccnc(N[C@@H]5CCc6ncccc65)c4s3)c1CO)C(=O)N[C@@]2(C)Cc1ccc(F)cc1] |
| 153 | 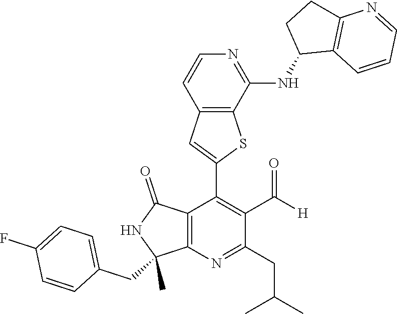 [SMILES: CC(C)Cc1nc2c(c(-c3cc4ccnc(N[C@@H]5CCc6ncccc65)c4s3)c1C=O)C(=O)N[C@@]2(C)Cc1ccc(F)cc1] |
| 154 | 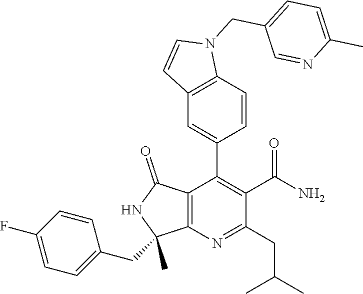 [SMILES: Cc1ccc(Cn2ccc3cc(-c4c(C(N)=O)c(CC(C)C)nc5c4C(=O)N[C@@]5(C)Cc4ccc(F)cc4)ccc32)cn1] |
| 155 | 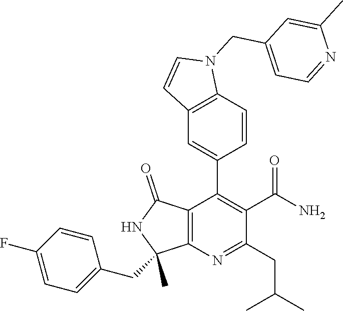 [SMILES: Cc1cc(Cn2ccc3cc(-c4c(C(N)=O)c(CC(C)C)nc5c4C(=O)N[C@@]5(C)Cc4ccc(F)cc4)ccc32)ccn1] |
| 156 | 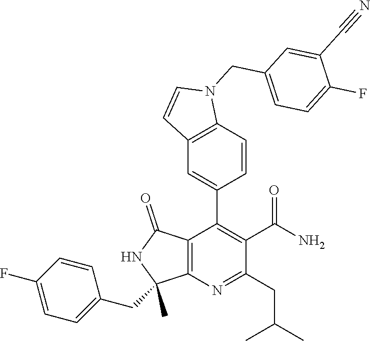 [SMILES: CC(C)Cc1nc2c(c(-c3ccc4c(ccn4Cc4ccc(F)c(C#N)c4)c3)c1C(N)=O)C(=O)N[C@@]2(C)Cc1ccc(F)cc1] |
| 157 | 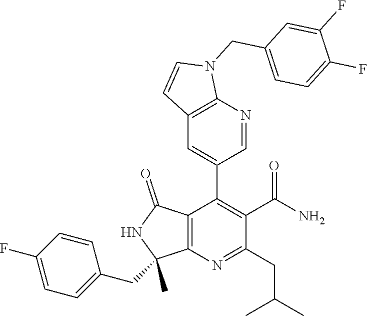 [SMILES: CC(C)Cc1nc2c(c(-c3cnc4c(ccn4Cc4ccc(F)c(F)c4)c3)c1C(N)=O)C(=O)N[C@@]2(C)Cc1ccc(F)cc1] |
| 158 | 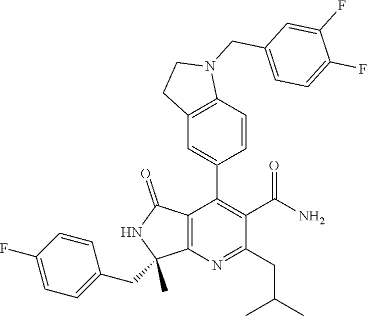 [SMILES: CC(C)Cc1nc2c(c(-c3ccc4c(c3)CCN4Cc3ccc(F)c(F)c3)c1C(N)=O)C(=O)N[C@@]2(C)Cc1ccc(F)cc1] |
| 159 | 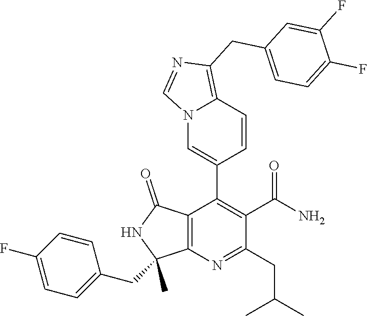 [SMILES: CC(C)Cc1nc2c(c(-c3ccc4c(Cc5ccc(F)c(F)c5)ncn4c3)c1C(N)=O)C(=O)N[C@@]2(C)Cc1ccc(F)cc1] |
| 160 | 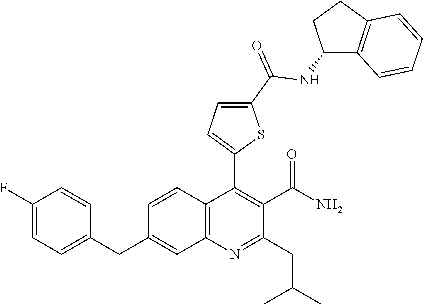 [SMILES: CC(C)Cc1nc2cc(Cc3ccc(F)cc3)ccc2c(-c2ccc(C(=O)N[C@@H]3CCc4ccccc43)s2)c1C(N)=O] |
| 161 | 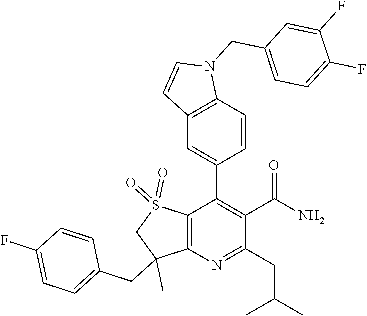 [SMILES: CC(C)Cc1nc2c(c(-c3ccc4c(ccn4Cc4ccc(F)c(F)c4)c3)c1C(N)=O)S(=O)(=O)CC2(C)Cc1ccc(F)cc1] |
| 162 | 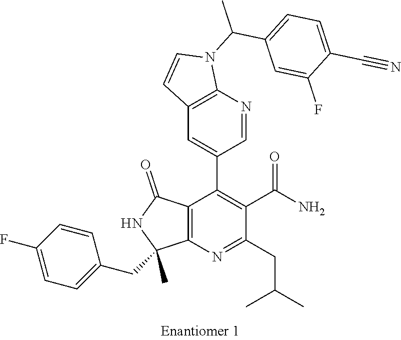 [SMILES: CC(C)Cc1nc2c(c(-c3cnc4c(ccn4C(C)c4ccc(C#N)c(F)c4)c3)c1C(N)=O)C(=O)N[C@@]2(C)Cc1ccc(F)cc1] |
| 163 | 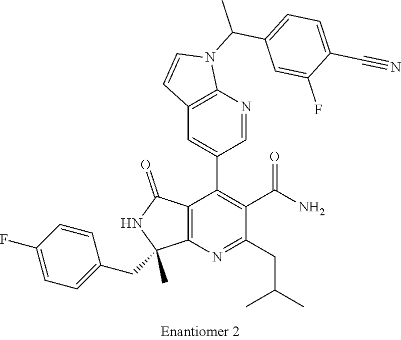 [SMILES: CC(C)Cc1nc2c(c(-c3cnc4c(ccn4C(C)c4ccc(C#N)c(F)c4)c3)c1C(N)=O)C(=O)N[C@@]2(C)Cc1ccc(F)cc1] |
| 164 | 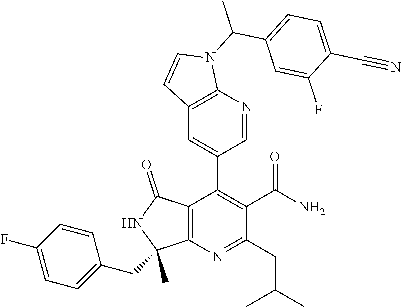 [SMILES: CC(C)Cc1nc2c(c(-c3cnc4c(ccn4C(C)c4ccc(C#N)c(F)c4)c3)c1C(N)=O)C(=O)N[C@@]2(C)Cc1ccc(F)cc1] |
| 165 | 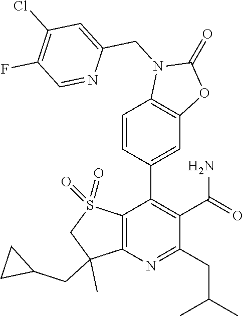 [SMILES: CC(C)Cc1nc2c(c(-c3ccc4c(c3)oc(=O)n4Cc3cc(Cl)c(F)cn3)c1C(N)=O)S(=O)(=O)CC2(C)CC1CC1] |
| 166 | 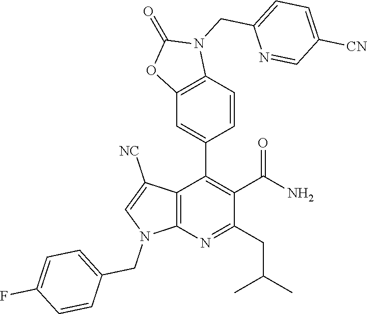 [SMILES: CC(C)Cc1nc2c(c(C#N)cn2Cc2ccc(F)cc2)c(-c2ccc3c(c2)oc(=O)n3Cc2ccc(C#N)cn2)c1C(N)=O] |
| 167 | 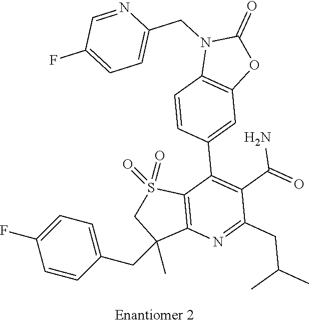 [SMILES: CC(C)Cc1nc2c(c(-c3ccc4c(c3)oc(=O)n4Cc3ccc(F)cn3)c1C(N)=O)S(=O)(=O)CC2(C)Cc1ccc(F)cc1] |
| 168 | 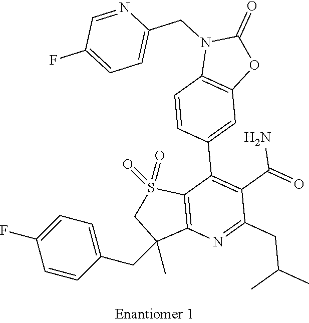 [SMILES: CC(C)Cc1nc2c(c(-c3ccc4c(c3)oc(=O)n4Cc3ccc(F)cn3)c1C(N)=O)S(=O)(=O)CC2(C)Cc1ccc(F)cc1] |
| 169 | 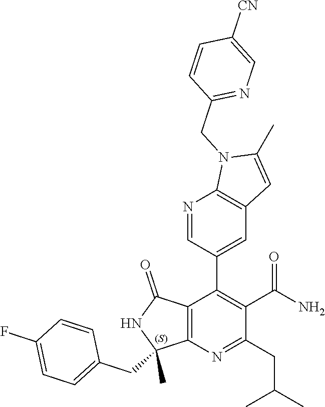 |
| 170 | 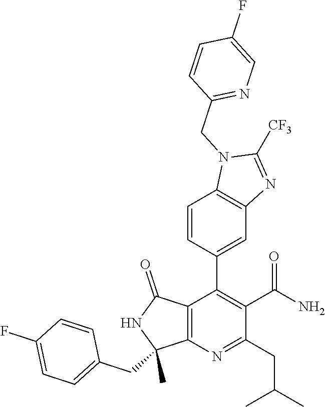 [SMILES: CC(C)Cc1nc2c(c(-c3ccc4c(c3)nc(C(F)(F)F)n4Cc3ccc(F)cn3)c1C(N)=O)C(=O)N[C@@]2(C)Cc1ccc(F)cc1] |
| 171 | 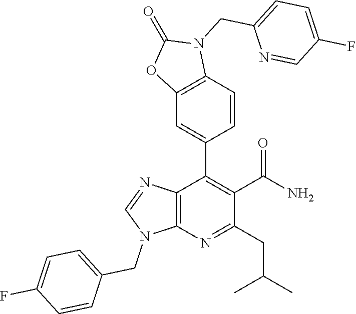 [SMILES: CC(C)Cc1nc2c(ncn2Cc2ccc(F)cc2)c(-c2ccc3c(c2)oc(=O)n3Cc2ccc(F)cn2)c1C(N)=O] |
| 172 | 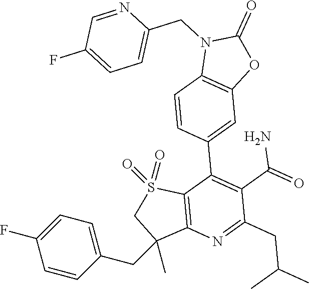 [SMILES: CC(C)Cc1nc2c(c(-c3ccc4c(c3)oc(=O)n4Cc3ccc(F)cn3)c1C(N)=O)S(=O)(=O)CC2(C)Cc1ccc(F)cc1] |
| 173 | 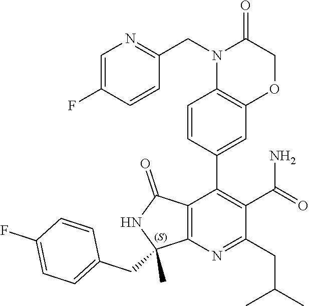 |
| 174 | 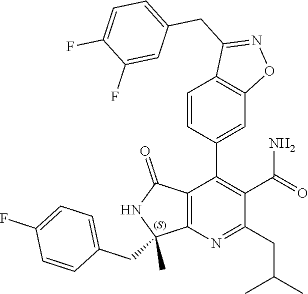 |
| 175 | 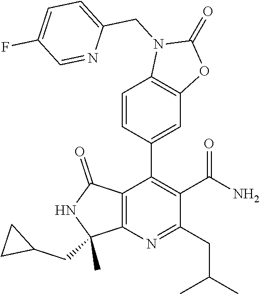 [SMILES: CC(C)Cc1nc2c(c(-c3ccc4c(c3)oc(=O)n4Cc3ccc(F)cn3)c1C(N)=O)C(=O)N[C@@]2(C)CC1CC1] |
| 176 | 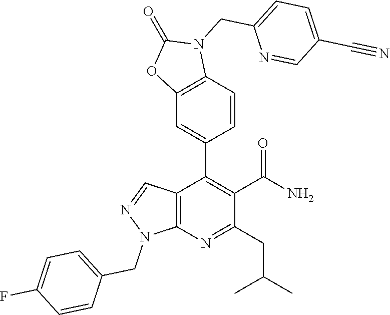 [SMILES: CC(C)Cc1nc2c(cnn2Cc2ccc(F)cc2)c(-c2ccc3c(c2)oc(=O)n3Cc2ccc(C#N)cn2)c1C(N)=O] |
| 177 | 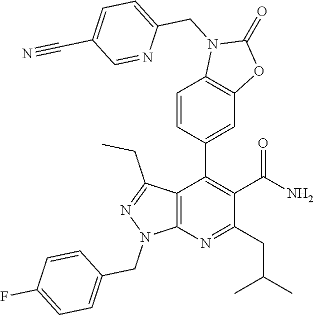 [SMILES: CCc1nn(Cc2ccc(F)cc2)c2nc(CC(C)C)c(C(N)=O)c(-c3ccc4c(c3)oc(=O)n4Cc3ccc(C#N)cn3)c12] |
| 178 | 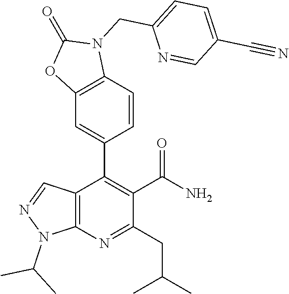 [SMILES: CC(C)Cc1nc2c(cnn2C(C)C)c(-c2ccc3c(c2)oc(=O)n3Cc2ccc(C#N)cn2)c1C(N)=O] |
| 179 | 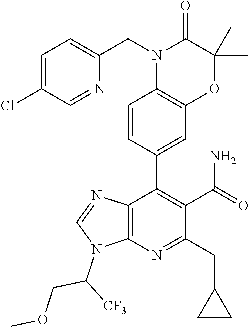 |
| 180 | 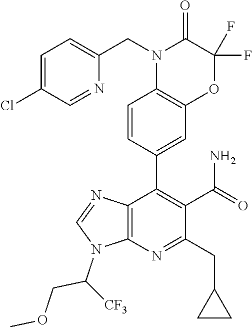 [SMILES: COCC(n1cnc2c(-c3ccc4c(c3)OC(F)(F)C(=O)N4Cc3ccc(Cl)cn3)c(C(N)=O)c(CC3CC3)nc21)C(F)(F)F] |
| 181 | 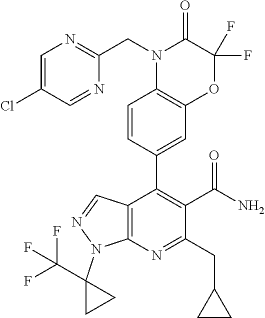 [SMILES: NC(=O)c1c(CC2CC2)nc2c(cnn2C2(C(F)(F)F)CC2)c1-c1ccc2c(c1)OC(F)(F)C(=O)N2Cc1ncc(Cl)cn1] |
| 182 | 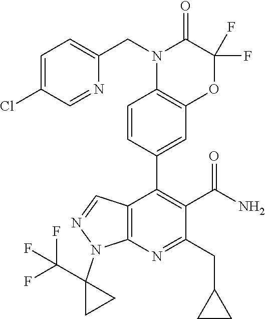 [SMILES: NC(=O)c1c(CC2CC2)nc2c(cnn2C2(C(F)(F)F)CC2)c1-c1ccc2c(c1)OC(F)(F)C(=O)N2Cc1ccc(Cl)cn1] |
| 183 | 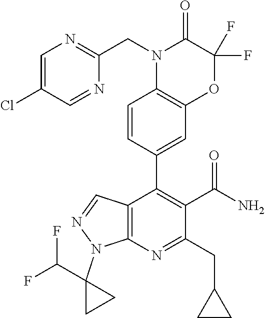 [SMILES: NC(=O)c1c(CC2CC2)nc2c(cnn2C2(C(F)F)CC2)c1-c1ccc2c(c1)OC(F)(F)C(=O)N2Cc1ncc(Cl)cn1] |
| 184 | 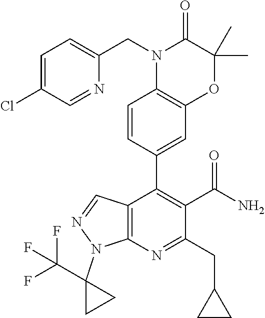 [SMILES: CC1(C)Oc2cc(-c3c(C(N)=O)c(CC4CC4)nc4c3cnn4C3(C(F)(F)F)CC3)ccc2N(Cc2ccc(Cl)cn2)C1=O] |
| 185 | 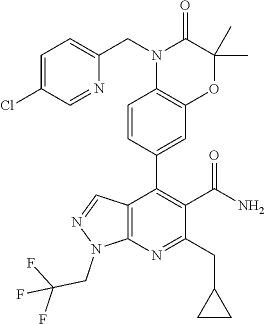 [SMILES: CC1(C)Oc2cc(-c3c(C(N)=O)c(CC4CC4)nc4c3cnn4CC(F)(F)F)ccc2N(Cc2ccc(Cl)cn2)C1=O] |
| 186 | 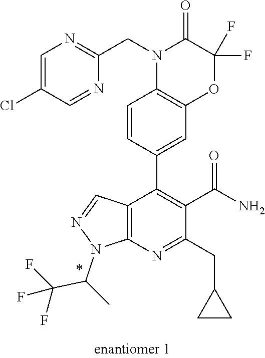 [SMILES: CC(n1ncc2c(-c3ccc4c(c3)OC(F)(F)C(=O)N4Cc3ncc(Cl)cn3)c(C(N)=O)c(CC3CC3)nc21)C(F)(F)F] |
| 187 | 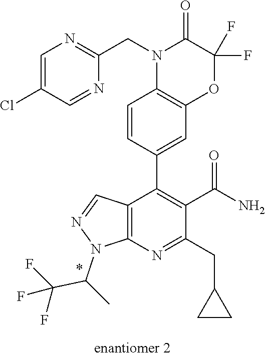 [SMILES: CC(n1ncc2c(-c3ccc4c(c3)OC(F)(F)C(=O)N4Cc3ncc(Cl)cn3)c(C(N)=O)c(CC3CC3)nc21)C(F)(F)F] |
| 188 | 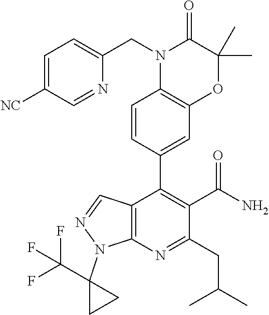 [SMILES: CC(C)Cc1nc2c(cnn2C2(C(F)(F)F)CC2)c(-c2ccc3c(c2)OC(C)(C)C(=O)N3Cc2ccc(C#N)cn2)c1C(N)=O] |
| 189 | 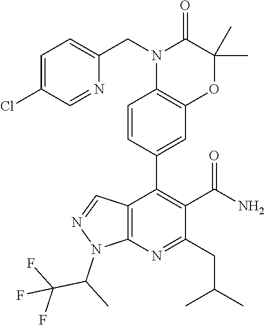 |
| 190 | 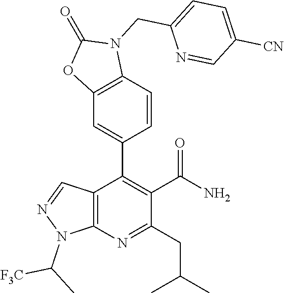 [SMILES: CC(C)Cc1nc2c(cnn2C(C)C(F)(F)F)c(-c2ccc3c(c2)oc(=O)n3Cc2ccc(C#N)cn2)c1C(N)=O] |
| 191 | 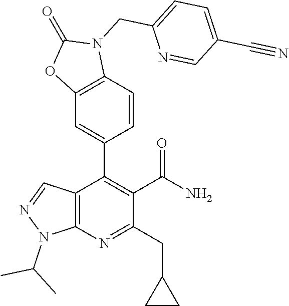 |
| 192 | 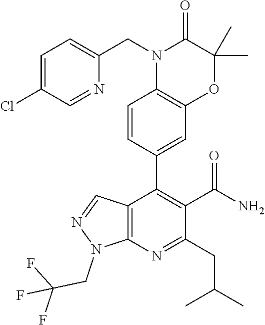 |
| 193 | 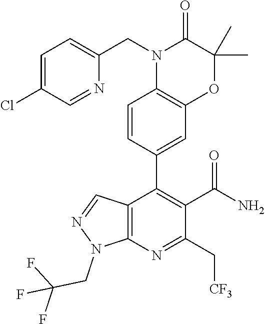 [SMILES: CC1(C)Oc2cc(-c3c(C(N)=O)c(CC(F)(F)F)nc4c3cnn4CC(F)(F)F)ccc2N(Cc2ccc(Cl)cn2)C1=O] |
| 194 | 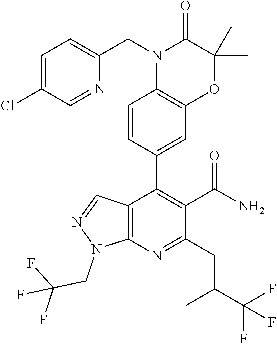 [SMILES: CC(Cc1nc2c(cnn2CC(F)(F)F)c(-c2ccc3c(c2)OC(C)(C)C(=O)N3Cc2ccc(Cl)cn2)c1C(N)=O)C(F)(F)F] |
| 195 | 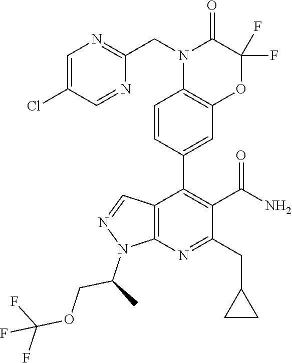 [SMILES: C[C@@H](COC(F)(F)F)n1ncc2c(-c3ccc4c(c3)OC(F)(F)C(=O)N4Cc3ncc(Cl)cn3)c(C(N)=O)c(CC3CC3)nc21] |
| 196 | 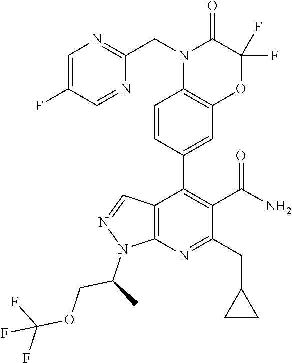 [SMILES: C[C@@H](COC(F)(F)F)n1ncc2c(-c3ccc4c(c3)OC(F)(F)C(=O)N4Cc3ncc(F)cn3)c(C(N)=O)c(CC3CC3)nc21] |
| 197 | 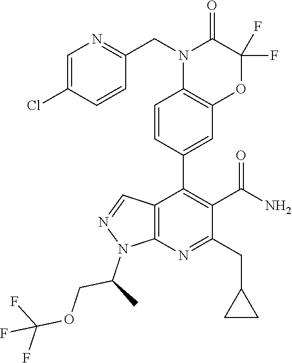 [SMILES: C[C@@H](COC(F)(F)F)n1ncc2c(-c3ccc4c(c3)OC(F)(F)C(=O)N4Cc3ccc(Cl)cn3)c(C(N)=O)c(CC3CC3)nc21] |
| 198 | 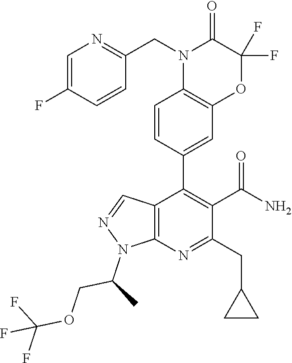 [SMILES: C[C@@H](COC(F)(F)F)n1ncc2c(-c3ccc4c(c3)OC(F)(F)C(=O)N4Cc3ccc(F)cn3)c(C(N)=O)c(CC3CC3)nc21] |
| 199 | 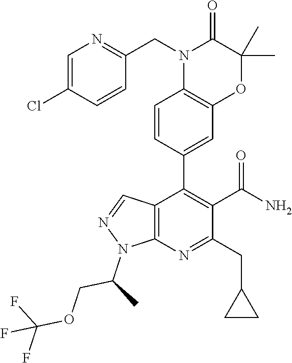 [SMILES: C[C@@H](COC(F)(F)F)n1ncc2c(-c3ccc4c(c3)OC(C)(C)C(=O)N4Cc3ccc(Cl)cn3)c(C(N)=O)c(CC3CC3)nc21] |
| 200 | 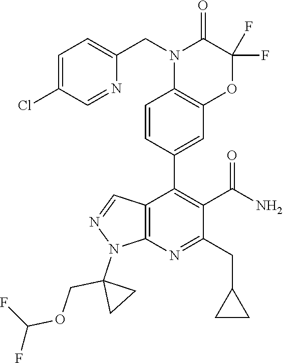 [SMILES: NC(=O)c1c(CC2CC2)nc2c(cnn2C2(COC(F)F)CC2)c1-c1ccc2c(c1)OC(F)(F)C(=O)N2Cc1ccc(Cl)cn1] |
| 201 | 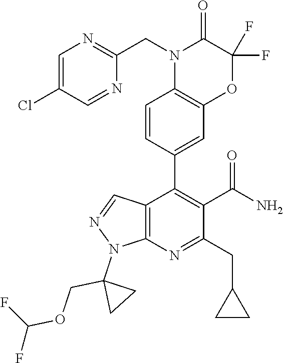 [SMILES: NC(=O)c1c(CC2CC2)nc2c(cnn2C2(COC(F)F)CC2)c1-c1ccc2c(c1)OC(F)(F)C(=O)N2Cc1ncc(Cl)cn1] |
| 207 | 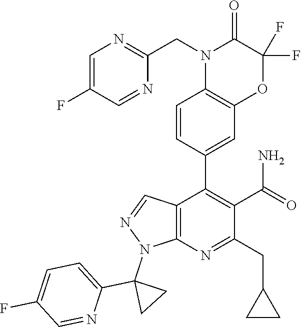 [SMILES: NC(=O)c1c(CC2CC2)nc2c(cnn2C2(c3ccc(F)cn3)CC2)c1-c1ccc2c(c1)OC(F)(F)C(=O)N2Cc1ncc(F)cn1] |
| 208 | 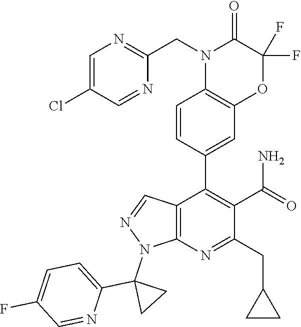 |
| 209 | 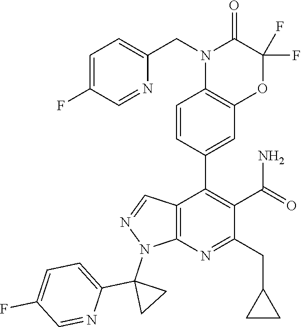 [SMILES: NC(=O)c1c(CC2CC2)nc2c(cnn2C2(c3ccc(F)cn3)CC2)c1-c1ccc2c(c1)OC(F)(F)C(=O)N2Cc1ccc(F)cn1] |
| 210 | 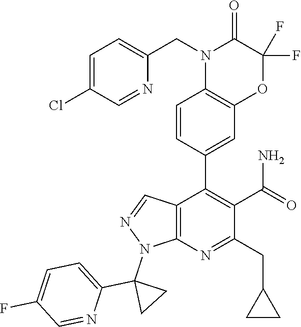 |
| 211 | 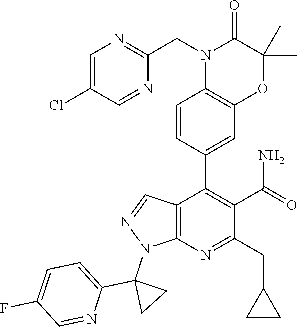 [SMILES: CC1(C)Oc2cc(-c3c(C(N)=O)c(CC4CC4)nc4c3cnn4C3(c4ccc(F)cn4)CC3)ccc2N(Cc2ncc(Cl)cn2)C1=O] |
| 212 | 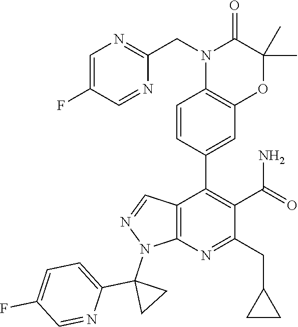 [SMILES: CC1(C)Oc2cc(-c3c(C(N)=O)c(CC4CC4)nc4c3cnn4C3(c4ccc(F)cn4)CC3)ccc2N(Cc2ncc(F)cn2)C1=O] |
| 213 | 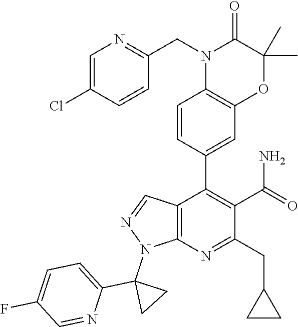 [SMILES: CC1(C)Oc2cc(-c3c(C(N)=O)c(CC4CC4)nc4c3cnn4C3(c4ccc(F)cn4)CC3)ccc2N(Cc2ccc(Cl)cn2)C1=O] |
| 214 | 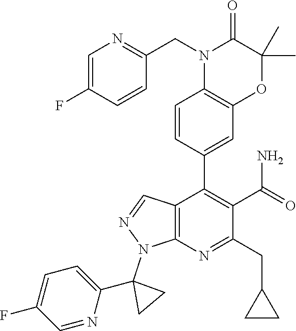 [SMILES: CC1(C)Oc2cc(-c3c(C(N)=O)c(CC4CC4)nc4c3cnn4C3(c4ccc(F)cn4)CC3)ccc2N(Cc2ccc(F)cn2)C1=O] |
| 215 | 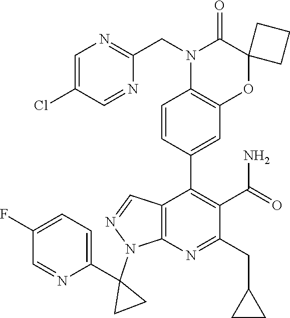 [SMILES: NC(=O)c1c(CC2CC2)nc2c(cnn2C2(c3ccc(F)cn3)CC2)c1-c1ccc2c(c1)OC1(CCC1)C(=O)N2Cc1ncc(Cl)cn1] |
| 216 | 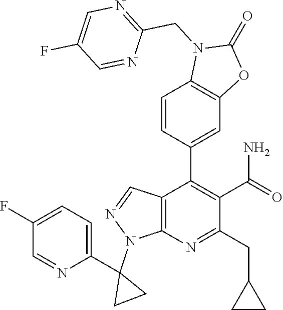 [SMILES: NC(=O)c1c(CC2CC2)nc2c(cnn2C2(c3ccc(F)cn3)CC2)c1-c1ccc2c(c1)oc(=O)n2Cc1ncc(F)cn1] |
| 217 | 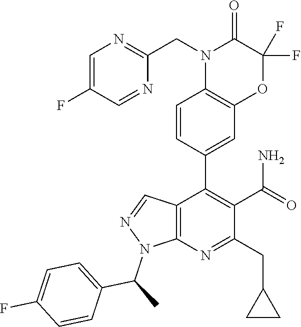 [SMILES: C[C@@H](c1ccc(F)cc1)n1ncc2c(-c3ccc4c(c3)OC(F)(F)C(=O)N4Cc3ncc(F)cn3)c(C(N)=O)c(CC3CC3)nc21] |
| 218 | 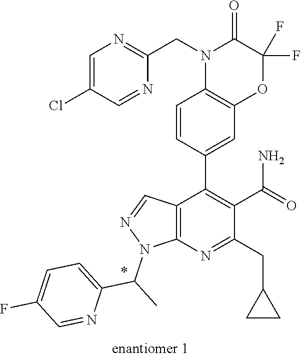 [SMILES: CC(c1ccc(F)cn1)n1ncc2c(-c3ccc4c(c3)OC(F)(F)C(=O)N4Cc3ncc(Cl)cn3)c(C(N)=O)c(CC3CC3)nc21] |
| 219 | 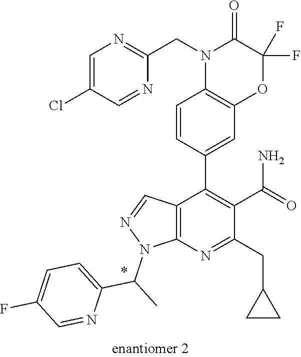 [SMILES: CC(c1ccc(F)cn1)n1ncc2c(-c3ccc4c(c3)OC(F)(F)C(=O)N4Cc3ncc(Cl)cn3)c(C(N)=O)c(CC3CC3)nc21] |
| 220 | 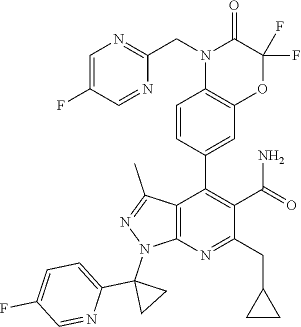 [SMILES: Cc1nn(C2(c3ccc(F)cn3)CC2)c2nc(CC3CC3)c(C(N)=O)c(-c3ccc4c(c3)OC(F)(F)C(=O)N4Cc3ncc(F)cn3)c12] |
| 221 | 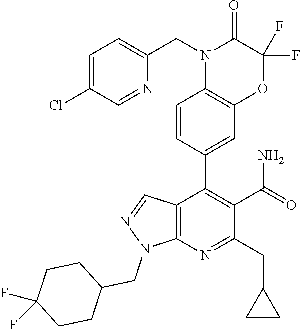 [SMILES: NC(=O)c1c(CC2CC2)nc2c(cnn2CC2CCC(F)(F)CC2)c1-c1ccc2c(c1)OC(F)(F)C(=O)N2Cc1ccc(Cl)cn1] |
| 222 | 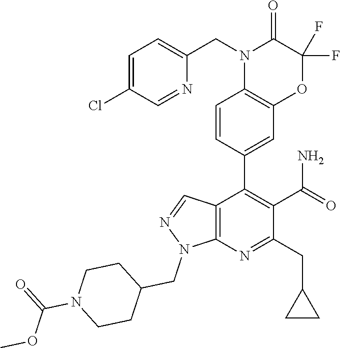 [SMILES: COC(=O)N1CCC(Cn2ncc3c(-c4ccc5c(c4)OC(F)(F)C(=O)N5Cc4ccc(Cl)cn4)c(C(N)=O)c(CC4CC4)nc32)CC1] |
| 223 | 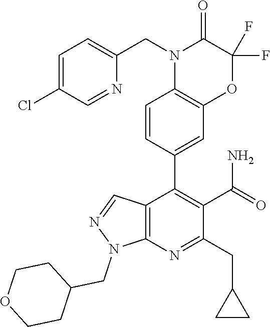 [SMILES: NC(=O)c1c(CC2CC2)nc2c(cnn2CC2CCOCC2)c1-c1ccc2c(c1)OC(F)(F)C(=O)N2Cc1ccc(Cl)cn1] |
| 224 | 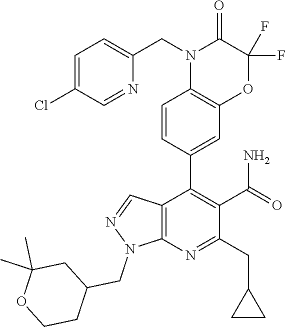 [SMILES: CC1(C)CC(Cn2ncc3c(-c4ccc5c(c4)OC(F)(F)C(=O)N5Cc4ccc(Cl)cn4)c(C(N)=O)c(CC4CC4)nc32)CCO1] |
| 225 | 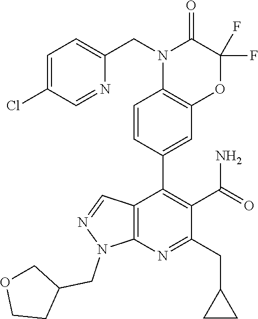 [SMILES: NC(=O)c1c(CC2CC2)nc2c(cnn2CC2CCOC2)c1-c1ccc2c(c1)OC(F)(F)C(=O)N2Cc1ccc(Cl)cn1] |
| 226 | 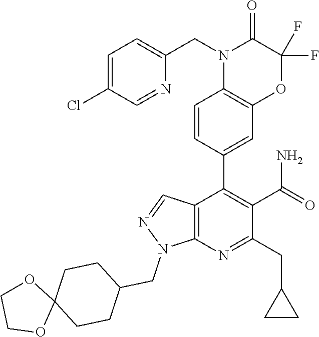 [SMILES: NC(=O)c1c(CC2CC2)nc2c(cnn2CC2CCC3(CC2)OCCO3)c1-c1ccc2c(c1)OC(F)(F)C(=O)N2Cc1ccc(Cl)cn1] |
| 227 | 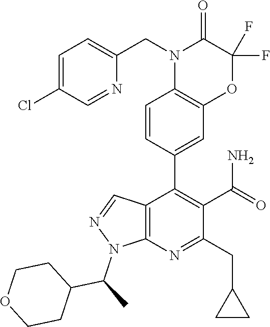 [SMILES: C[C@@H](C1CCOCC1)n1ncc2c(-c3ccc4c(c3)OC(F)(F)C(=O)N4Cc3ccc(Cl)cn3)c(C(N)=O)c(CC3CC3)nc21] |
| 228 | 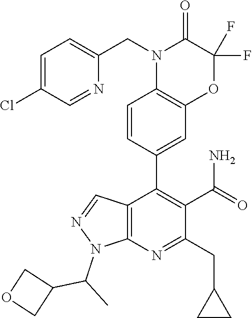 [SMILES: CC(C1COC1)n1ncc2c(-c3ccc4c(c3)OC(F)(F)C(=O)N4Cc3ccc(Cl)cn3)c(C(N)=O)c(CC3CC3)nc21] |
| 229 | 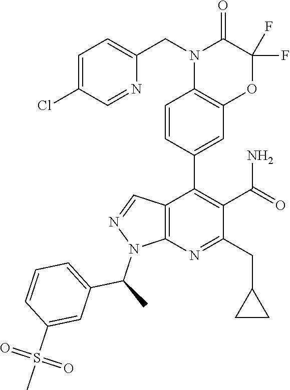 [SMILES: C[C@@H](c1cccc(S(C)(=O)=O)c1)n1ncc2c(-c3ccc4c(c3)OC(F)(F)C(=O)N4Cc3ccc(Cl)cn3)c(C(N)=O)c(CC3CC3)nc21] |
| 230 | 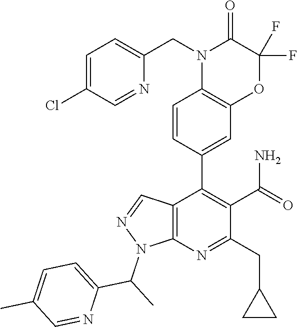 [SMILES: Cc1ccc(C(C)n2ncc3c(-c4ccc5c(c4)OC(F)(F)C(=O)N5Cc4ccc(Cl)cn4)c(C(N)=O)c(CC4CC4)nc32)nc1] |
| 231 | 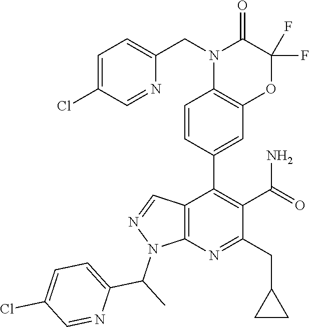 [SMILES: CC(c1ccc(Cl)cn1)n1ncc2c(-c3ccc4c(c3)OC(F)(F)C(=O)N4Cc3ccc(Cl)cn3)c(C(N)=O)c(CC3CC3)nc21] |
| 232 | 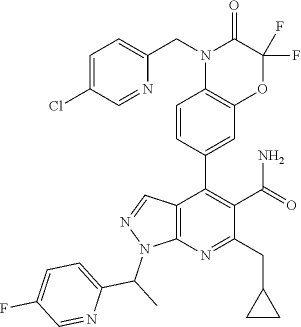 [SMILES: CC(c1ccc(F)cn1)n1ncc2c(-c3ccc4c(c3)OC(F)(F)C(=O)N4Cc3ccc(Cl)cn3)c(C(N)=O)c(CC3CC3)nc21] |
| 233 | 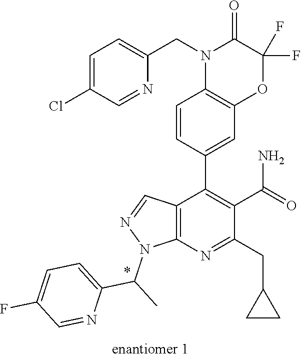 [SMILES: CC(c1ccc(F)cn1)n1ncc2c(-c3ccc4c(c3)OC(F)(F)C(=O)N4Cc3ccc(Cl)cn3)c(C(N)=O)c(CC3CC3)nc21] |
| 234 | 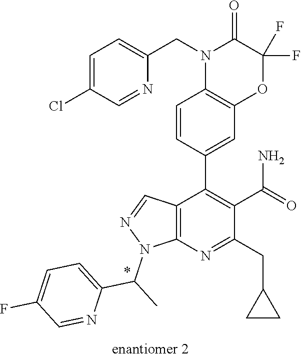 [SMILES: CC(c1ccc(F)cn1)n1ncc2c(-c3ccc4c(c3)OC(F)(F)C(=O)N4Cc3ccc(Cl)cn3)c(C(N)=O)c(CC3CC3)nc21] |
| 235 | 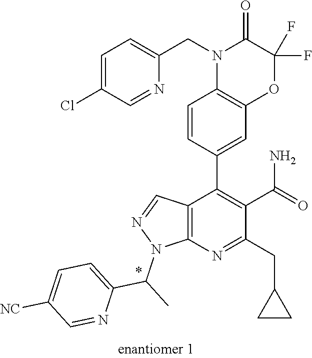 [SMILES: CC(c1ccc(C#N)cn1)n1ncc2c(-c3ccc4c(c3)OC(F)(F)C(=O)N4Cc3ccc(Cl)cn3)c(C(N)=O)c(CC3CC3)nc21] |
| 236 | 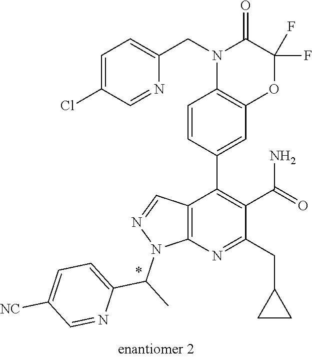 [SMILES: CC(c1ccc(C#N)cn1)n1ncc2c(-c3ccc4c(c3)OC(F)(F)C(=O)N4Cc3ccc(Cl)cn3)c(C(N)=O)c(CC3CC3)nc21] |
| 237 | 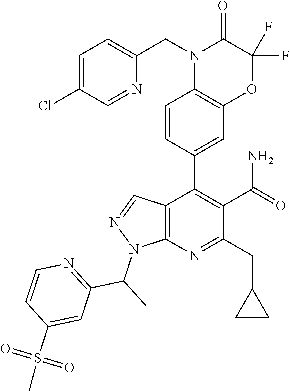 [SMILES: CC(c1cc(S(C)(=O)=O)ccn1)n1ncc2c(-c3ccc4c(c3)OC(F)(F)C(=O)N4Cc3ccc(Cl)cn3)c(C(N)=O)c(CC3CC3)nc21] |
| 238 | 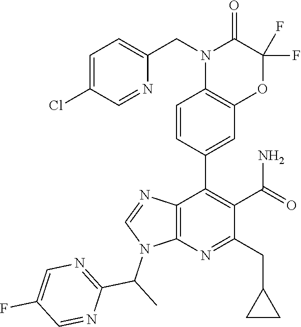 [SMILES: CC(c1ncc(F)cn1)n1cnc2c(-c3ccc4c(c3)OC(F)(F)C(=O)N4Cc3ccc(Cl)cn3)c(C(N)=O)c(CC3CC3)nc21] |
| 239 | 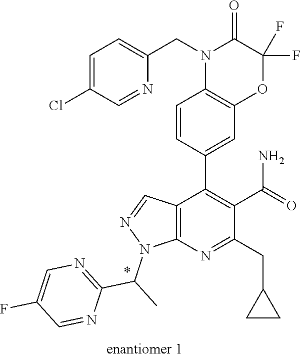 [SMILES: CC(c1ncc(F)cn1)n1ncc2c(-c3ccc4c(c3)OC(F)(F)C(=O)N4Cc3ccc(Cl)cn3)c(C(N)=O)c(CC3CC3)nc21] |
| 240 | 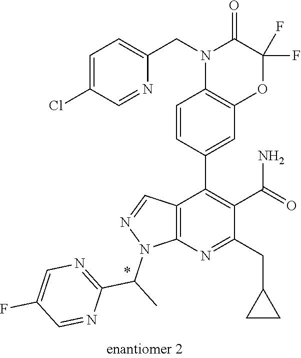 [SMILES: CC(c1ncc(F)cn1)n1ncc2c(-c3ccc4c(c3)OC(F)(F)C(=O)N4Cc3ccc(Cl)cn3)c(C(N)=O)c(CC3CC3)nc21] |
| 241 | 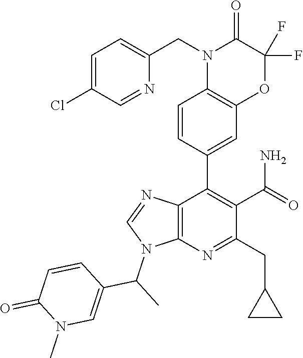 [SMILES: CC(c1ccc(=O)n(C)c1)n1cnc2c(-c3ccc4c(c3)OC(F)(F)C(=O)N4Cc3ccc(Cl)cn3)c(C(N)=O)c(CC3CC3)nc21] |
| 242 | 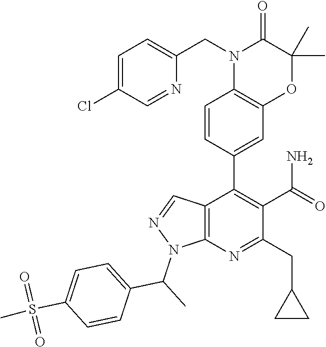 |
| 243 | 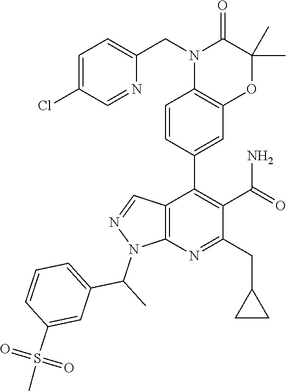 [SMILES: CC(c1cccc(S(C)(=O)=O)c1)n1ncc2c(-c3ccc4c(c3)OC(C)(C)C(=O)N4Cc3ccc(Cl)cn3)c(C(N)=O)c(CC3CC3)nc21] |
| 244 | 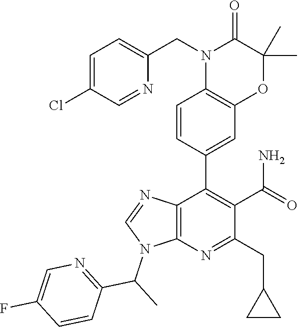 [SMILES: CC(c1ccc(F)cn1)n1cnc2c(-c3ccc4c(c3)OC(C)(C)C(=O)N4Cc3ccc(Cl)cn3)c(C(N)=O)c(CC3CC3)nc21] |
| 245 | 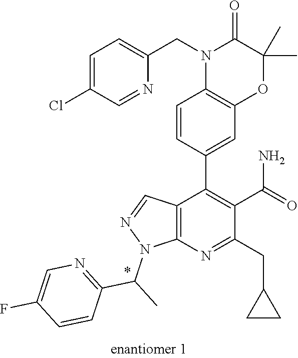 [SMILES: CC(c1ccc(F)cn1)n1ncc2c(-c3ccc4c(c3)OC(C)(C)C(=O)N4Cc3ccc(Cl)cn3)c(C(N)=O)c(CC3CC3)nc21] |
| 246 | 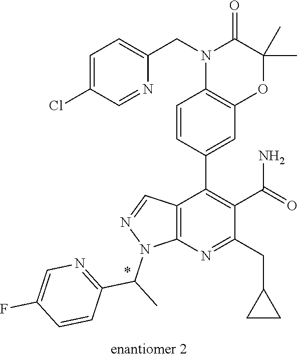 [SMILES: CC(c1ccc(F)cn1)n1ncc2c(-c3ccc4c(c3)OC(C)(C)C(=O)N4Cc3ccc(Cl)cn3)c(C(N)=O)c(CC3CC3)nc21] |
| 247 | 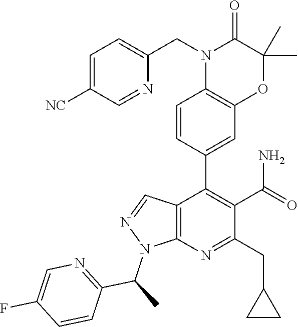 [SMILES: C[C@@H](c1ccc(F)cn1)n1ncc2c(-c3ccc4c(c3)OC(C)(C)C(=O)N4Cc3ccc(C#N)cn3)c(C(N)=O)c(CC3CC3)nc21] |
| 248 | 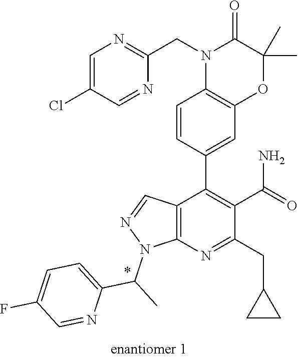 [SMILES: CC(c1ccc(F)cn1)n1ncc2c(-c3ccc4c(c3)OC(C)(C)C(=O)N4Cc3ncc(Cl)cn3)c(C(N)=O)c(CC3CC3)nc21] |
| 249 | 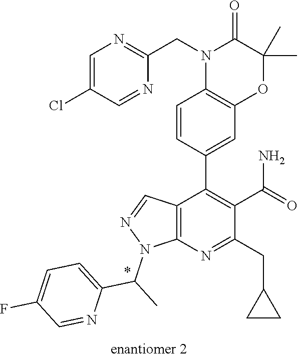 [SMILES: CC(c1ccc(F)cn1)n1ncc2c(-c3ccc4c(c3)OC(C)(C)C(=O)N4Cc3ncc(Cl)cn3)c(C(N)=O)c(CC3CC3)nc21] |
| 250 | 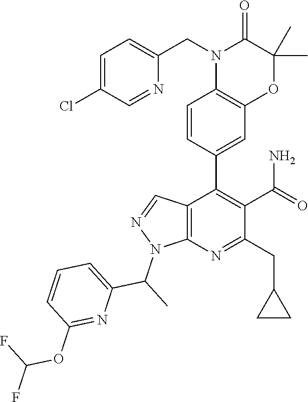 [SMILES: CC(c1cccc(OC(F)F)n1)n1ncc2c(-c3ccc4c(c3)OC(C)(C)C(=O)N4Cc3ccc(Cl)cn3)c(C(N)=O)c(CC3CC3)nc21] |
| 251 | 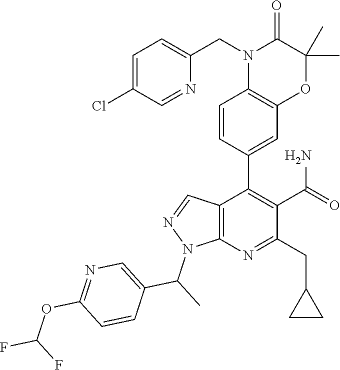 [SMILES: CC(c1ccc(OC(F)F)nc1)n1ncc2c(-c3ccc4c(c3)OC(C)(C)C(=O)N4Cc3ccc(Cl)cn3)c(C(N)=O)c(CC3CC3)nc21] |
| 252 | 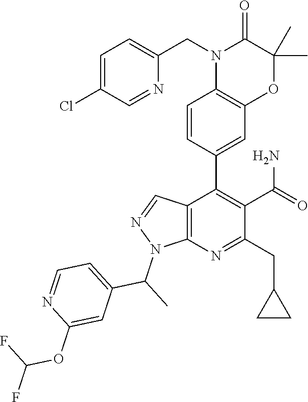 [SMILES: CC(c1ccnc(OC(F)F)c1)n1ncc2c(-c3ccc4c(c3)OC(C)(C)C(=O)N4Cc3ccc(Cl)cn3)c(C(N)=O)c(CC3CC3)nc21] |
| 253 | 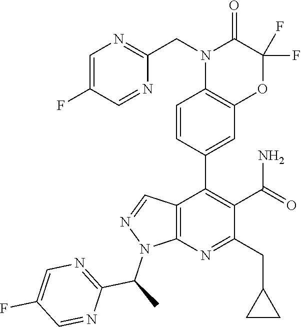 [SMILES: C[C@@H](c1ncc(F)cn1)n1ncc2c(-c3ccc4c(c3)OC(F)(F)C(=O)N4Cc3ncc(F)cn3)c(C(N)=O)c(CC3CC3)nc21] |
| 254 | 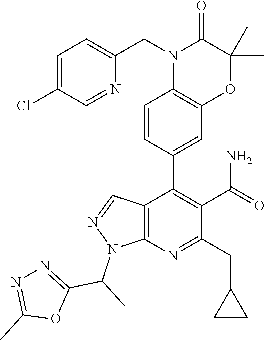 |
| 255 | 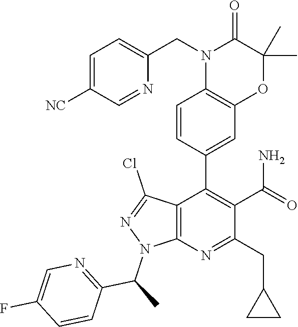 [SMILES: C[C@@H](c1ccc(F)cn1)n1nc(Cl)c2c(-c3ccc4c(c3)OC(C)(C)C(=O)N4Cc3ccc(C#N)cn3)c(C(N)=O)c(CC3CC3)nc21] |
| 256 | 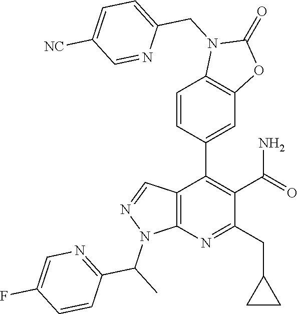 [SMILES: CC(c1ccc(F)cn1)n1ncc2c(-c3ccc4c(c3)oc(=O)n4Cc3ccc(C#N)cn3)c(C(N)=O)c(CC3CC3)nc21] |
| 257 | 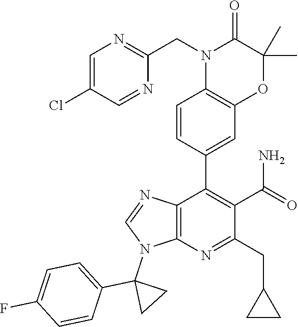 [SMILES: CC1(C)Oc2cc(-c3c(C(N)=O)c(CC4CC4)nc4c3ncn4C3(c4ccc(F)cc4)CC3)ccc2N(Cc2ncc(Cl)cn2)C1=O] |
| 258 | 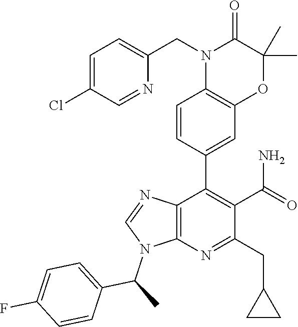 [SMILES: C[C@@H](c1ccc(F)cc1)n1cnc2c(-c3ccc4c(c3)OC(C)(C)C(=O)N4Cc3ccc(Cl)cn3)c(C(N)=O)c(CC3CC3)nc21] |
| 259 | 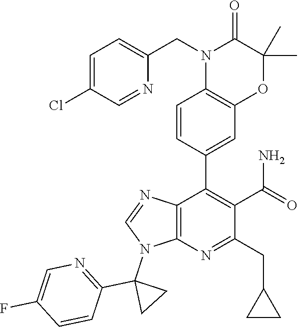 [SMILES: CC1(C)Oc2cc(-c3c(C(N)=O)c(CC4CC4)nc4c3ncn4C3(c4ccc(F)cn4)CC3)ccc2N(Cc2ccc(Cl)cn2)C1=O] |
| 260 | 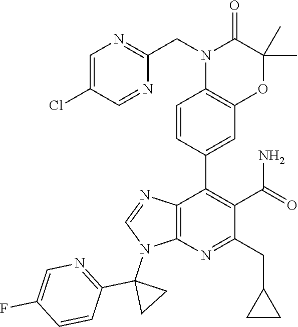 [SMILES: CC1(C)Oc2cc(-c3c(C(N)=O)c(CC4CC4)nc4c3ncn4C3(c4ccc(F)cn4)CC3)ccc2N(Cc2ncc(Cl)cn2)C1=O] |
| 261 | 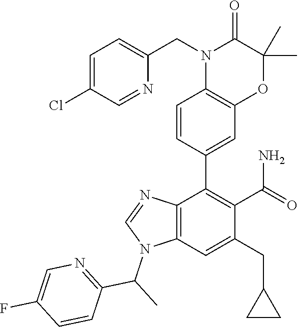 [SMILES: CC(c1ccc(F)cn1)n1cnc2c(-c3ccc4c(c3)OC(C)(C)C(=O)N4Cc3ccc(Cl)cn3)c(C(N)=O)c(CC3CC3)cc21] |
| 262 | 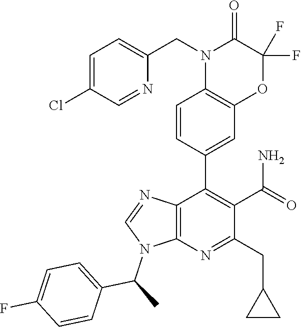 [SMILES: C[C@@H](c1ccc(F)cc1)n1cnc2c(-c3ccc4c(c3)OC(F)(F)C(=O)N4Cc3ccc(Cl)cn3)c(C(N)=O)c(CC3CC3)nc21] |
| 263 | 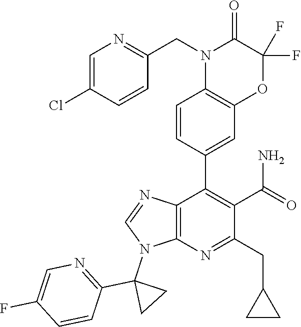 [SMILES: NC(=O)c1c(CC2CC2)nc2c(ncn2C2(c3ccc(F)cn3)CC2)c1-c1ccc2c(c1)OC(F)(F)C(=O)N2Cc1ccc(Cl)cn1] |
| 264 | 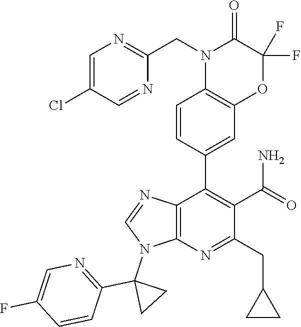 [SMILES: NC(=O)c1c(CC2CC2)nc2c(ncn2C2(c3ccc(F)cn3)CC2)c1-c1ccc2c(c1)OC(F)(F)C(=O)N2Cc1ncc(Cl)cn1] |
| 265 | 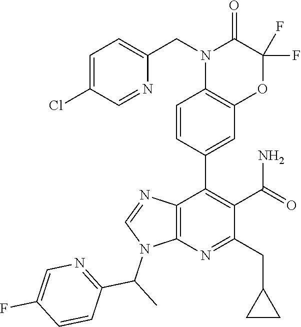 |
| 266 | 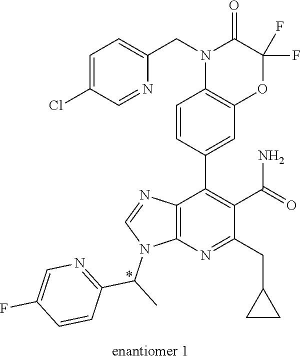 [SMILES: CC(c1ccc(F)cn1)n1cnc2c(-c3ccc4c(c3)OC(F)(F)C(=O)N4Cc3ccc(Cl)cn3)c(C(N)=O)c(CC3CC3)nc21] |
| 267 | 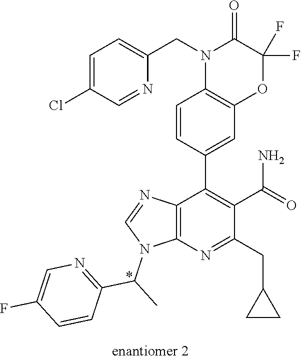 [SMILES: CC(c1ccc(F)cn1)n1cnc2c(-c3ccc4c(c3)OC(F)(F)C(=O)N4Cc3ccc(Cl)cn3)c(C(N)=O)c(CC3CC3)nc21] |
| 268 | 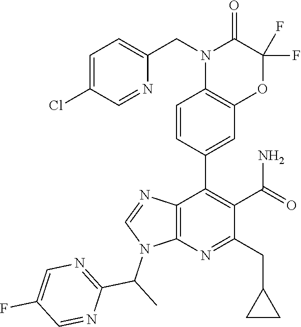 [SMILES: CC(c1ncc(F)cn1)n1cnc2c(-c3ccc4c(c3)OC(F)(F)C(=O)N4Cc3ccc(Cl)cn3)c(C(N)=O)c(CC3CC3)nc21] |
| 269 | 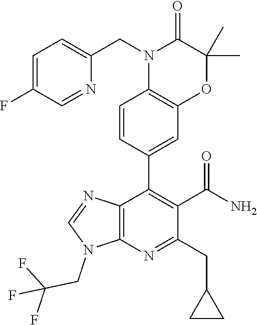 [SMILES: CC1(C)Oc2cc(-c3c(C(N)=O)c(CC4CC4)nc4c3ncn4CC(F)(F)F)ccc2N(Cc2ccc(F)cn2)C1=O] |
| 270 | 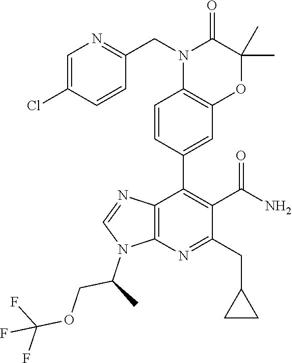 [SMILES: C[C@@H](COC(F)(F)F)n1cnc2c(-c3ccc4c(c3)OC(C)(C)C(=O)N4Cc3ccc(Cl)cn3)c(C(N)=O)c(CC3CC3)nc21] |
| 271 | 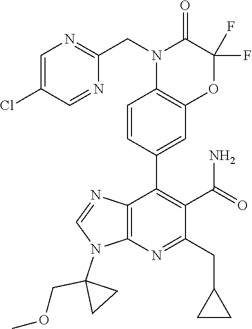 [SMILES: COCC1(n2cnc3c(-c4ccc5c(c4)OC(F)(F)C(=O)N5Cc4ncc(Cl)cn4)c(C(N)=O)c(CC4CC4)nc32)CC1] |
| 272 | 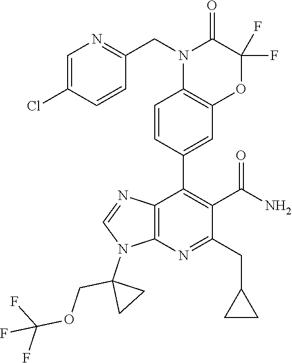 [SMILES: NC(=O)c1c(CC2CC2)nc2c(ncn2C2(COC(F)(F)F)CC2)c1-c1ccc2c(c1)OC(F)(F)C(=O)N2Cc1ccc(Cl)cn1] |
| 273 | 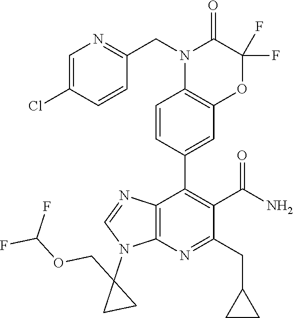 [SMILES: NC(=O)c1c(CC2CC2)nc2c(ncn2C2(COC(F)F)CC2)c1-c1ccc2c(c1)OC(F)(F)C(=O)N2Cc1ccc(Cl)cn1] |
| 274 | 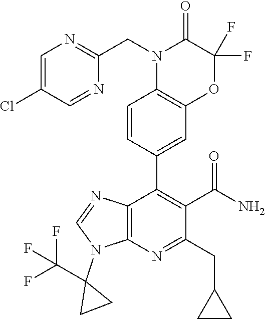 [SMILES: NC(=O)c1c(CC2CC2)nc2c(ncn2C2(C(F)(F)F)CC2)c1-c1ccc2c(c1)OC(F)(F)C(=O)N2Cc1ncc(Cl)cn1] |
| 275 | 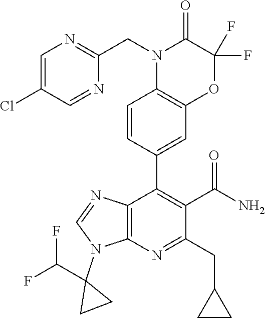 [SMILES: NC(=O)c1c(CC2CC2)nc2c(ncn2C2(C(F)F)CC2)c1-c1ccc2c(c1)OC(F)(F)C(=O)N2Cc1ncc(Cl)cn1] |
| 276 | 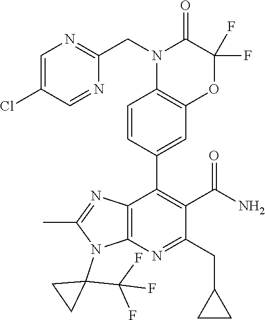 [SMILES: Cc1nc2c(-c3ccc4c(c3)OC(F)(F)C(=O)N4Cc3ncc(Cl)cn3)c(C(N)=O)c(CC3CC3)nc2n1C1(C(F)(F)F)CC1] |
| 278 | 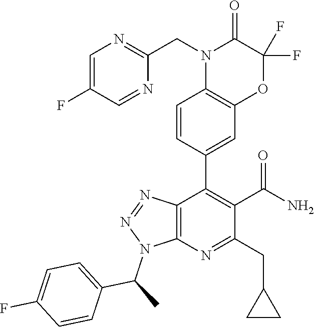 [SMILES: C[C@@H](c1ccc(F)cc1)n1nnc2c(-c3ccc4c(c3)OC(F)(F)C(=O)N4Cc3ncc(F)cn3)c(C(N)=O)c(CC3CC3)nc21] |
| 279 | 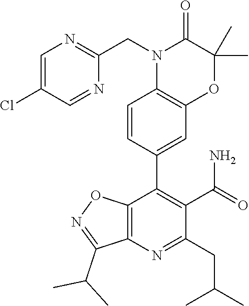 [SMILES: CC(C)Cc1nc2c(C(C)C)noc2c(-c2ccc3c(c2)OC(C)(C)C(=O)N3Cc2ncc(Cl)cn2)c1C(N)=O] |
| 280 | 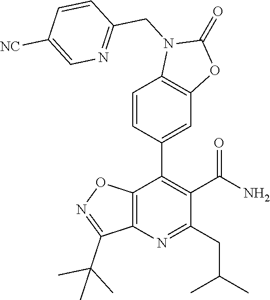 [SMILES: CC(C)Cc1nc2c(C(C)(C)C)noc2c(-c2ccc3c(c2)oc(=O)n3Cc2ccc(C#N)cn2)c1C(N)=O] |
| 281 | 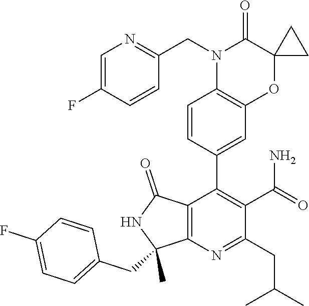 [SMILES: CC(C)Cc1nc2c(c(-c3ccc4c(c3)OC3(CC3)C(=O)N4Cc3ccc(F)cn3)c1C(N)=O)C(=O)N[C@@]2(C)Cc1ccc(F)cc1] |
| 282 | 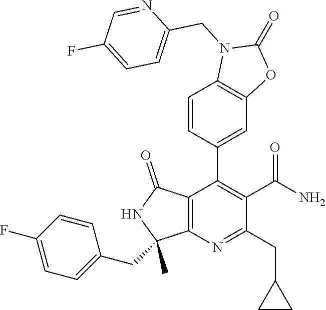 [SMILES: C[C@@]1(Cc2ccc(F)cc2)NC(=O)c2c1nc(CC1CC1)c(C(N)=O)c2-c1ccc2c(c1)oc(=O)n2Cc1ccc(F)cn1] |
| 283 | 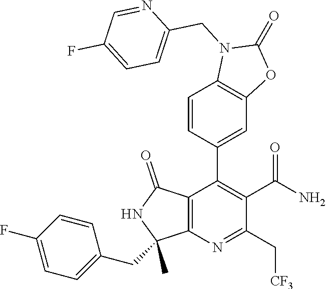 [SMILES: C[C@@]1(Cc2ccc(F)cc2)NC(=O)c2c1nc(CC(F)(F)F)c(C(N)=O)c2-c1ccc2c(c1)oc(=O)n2Cc1ccc(F)cn1] |
| 284 | 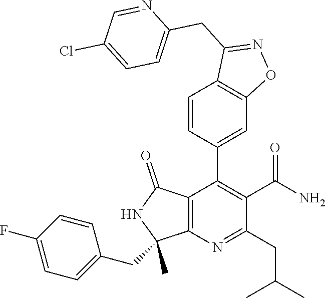 [SMILES: CC(C)Cc1nc2c(c(-c3ccc4c(Cc5ccc(Cl)cn5)noc4c3)c1C(N)=O)C(=O)N[C@@]2(C)Cc1ccc(F)cc1] |
| 285 | 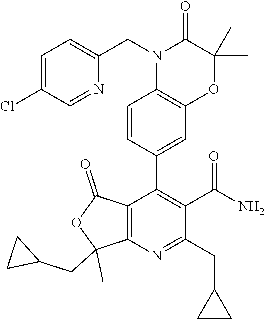 [SMILES: CC1(C)Oc2cc(-c3c(C(N)=O)c(CC4CC4)nc4c3C(=O)OC4(C)CC3CC3)ccc2N(Cc2ccc(Cl)cn2)C1=O] |
| 286 | 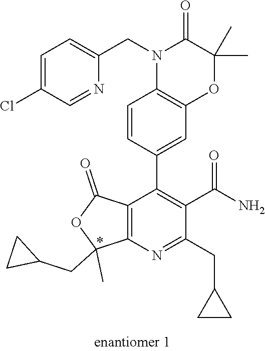 [SMILES: CC1(C)Oc2cc(-c3c(C(N)=O)c(CC4CC4)nc4c3C(=O)OC4(C)CC3CC3)ccc2N(Cc2ccc(Cl)cn2)C1=O] |
| 287 | 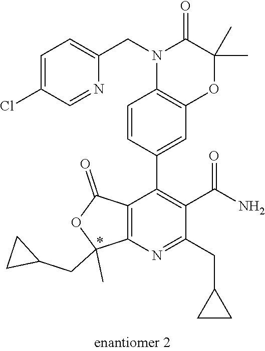 [SMILES: CC1(C)Oc2cc(-c3c(C(N)=O)c(CC4CC4)nc4c3C(=O)OC4(C)CC3CC3)ccc2N(Cc2ccc(Cl)cn2)C1=O] |
| 288 | 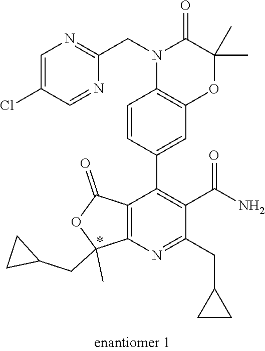 [SMILES: CC1(C)Oc2cc(-c3c(C(N)=O)c(CC4CC4)nc4c3C(=O)OC4(C)CC3CC3)ccc2N(Cc2ncc(Cl)cn2)C1=O] |
| 289 | 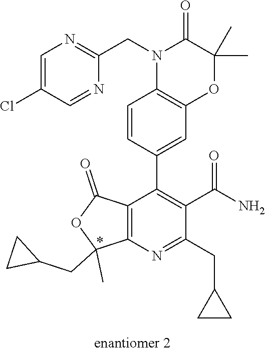 [SMILES: CC1(C)Oc2cc(-c3c(C(N)=O)c(CC4CC4)nc4c3C(=O)OC4(C)CC3CC3)ccc2N(Cc2ncc(Cl)cn2)C1=O] |
| 290 | 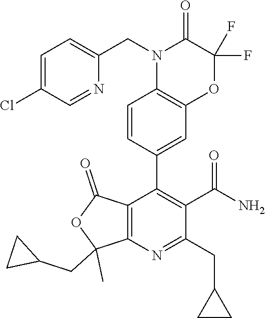 [SMILES: CC1(CC2CC2)OC(=O)c2c1nc(CC1CC1)c(C(N)=O)c2-c1ccc2c(c1)OC(F)(F)C(=O)N2Cc1ccc(Cl)cn1] |
| 291 | 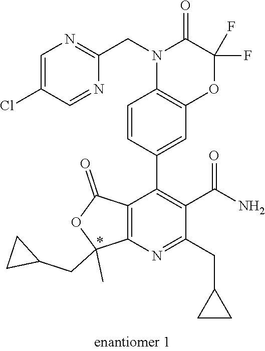 [SMILES: CC1(CC2CC2)OC(=O)c2c1nc(CC1CC1)c(C(N)=O)c2-c1ccc2c(c1)OC(F)(F)C(=O)N2Cc1ncc(Cl)cn1] |
| 292 | 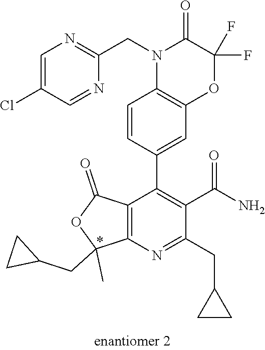 [SMILES: CC1(CC2CC2)OC(=O)c2c1nc(CC1CC1)c(C(N)=O)c2-c1ccc2c(c1)OC(F)(F)C(=O)N2Cc1ncc(Cl)cn1] |
| 293 | 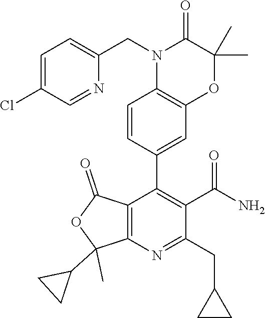 [SMILES: CC1(C)Oc2cc(-c3c(C(N)=O)c(CC4CC4)nc4c3C(=O)OC4(C)C3CC3)ccc2N(Cc2ccc(Cl)cn2)C1=O] |
| 294 | 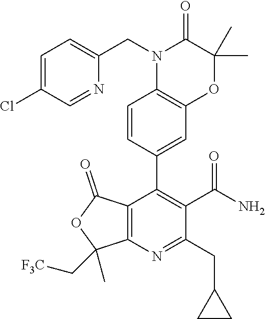 [SMILES: CC1(C)Oc2cc(-c3c(C(N)=O)c(CC4CC4)nc4c3C(=O)OC4(C)CC(F)(F)F)ccc2N(Cc2ccc(Cl)cn2)C1=O] |
| 295 | 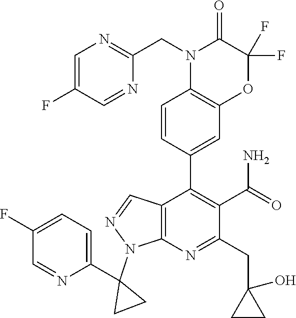 [SMILES: NC(=O)c1c(CC2(O)CC2)nc2c(cnn2C2(c3ccc(F)cn3)CC2)c1-c1ccc2c(c1)OC(F)(F)C(=O)N2Cc1ncc(F)cn1] |
| 296 | 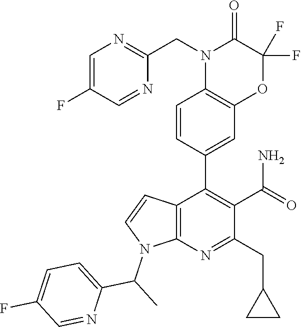 [SMILES: CC(c1ccc(F)cn1)n1ccc2c(-c3ccc4c(c3)OC(F)(F)C(=O)N4Cc3ncc(F)cn3)c(C(N)=O)c(CC3CC3)nc21] |
| 297 | 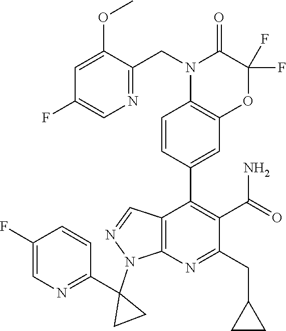 [SMILES: COc1cc(F)cnc1CN1C(=O)C(F)(F)Oc2cc(-c3c(C(N)=O)c(CC4CC4)nc4c3cnn4C3(c4ccc(F)cn4)CC3)ccc21] |
| 298 | 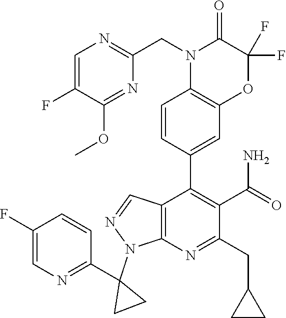 [SMILES: COc1nc(CN2C(=O)C(F)(F)Oc3cc(-c4c(C(N)=O)c(CC5CC5)nc5c4cnn5C4(c5ccc(F)cn5)CC4)ccc32)ncc1F] |
| 299 | 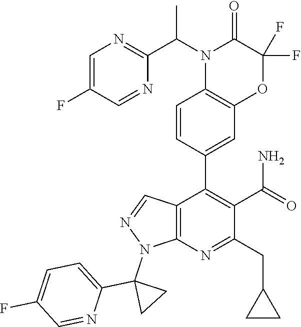 |
| 300 | 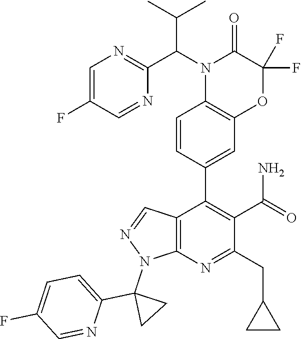 [SMILES: CC(C)C(c1ncc(F)cn1)N1C(=O)C(F)(F)Oc2cc(-c3c(C(N)=O)c(CC4CC4)nc4c3cnn4C3(c4ccc(F)cn4)CC3)ccc21] |
| 301 | 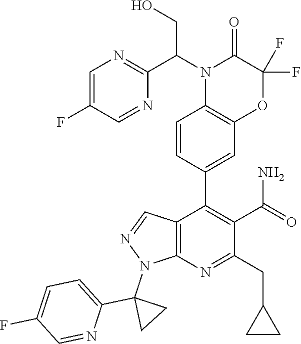 [SMILES: NC(=O)c1c(CC2CC2)nc2c(cnn2C2(c3ccc(F)cn3)CC2)c1-c1ccc2c(c1)OC(F)(F)C(=O)N2C(CO)c1ncc(F)cn1] |
| 302 | 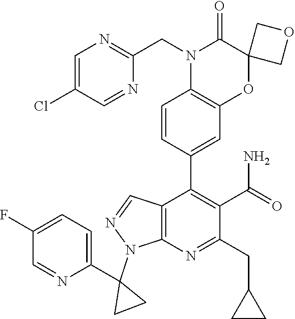 [SMILES: NC(=O)c1c(CC2CC2)nc2c(cnn2C2(c3ccc(F)cn3)CC2)c1-c1ccc2c(c1)OC1(COC1)C(=O)N2Cc1ncc(Cl)cn1] |
| 303 | 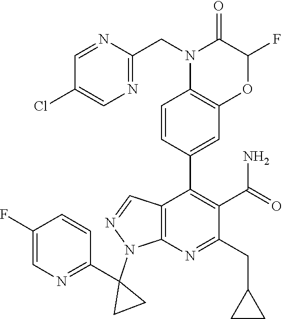 [SMILES: NC(=O)c1c(CC2CC2)nc2c(cnn2C2(c3ccc(F)cn3)CC2)c1-c1ccc2c(c1)OC(F)C(=O)N2Cc1ncc(Cl)cn1] |
| 304 | 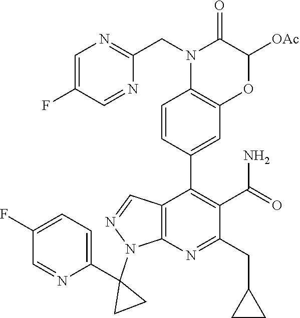 [SMILES: CC(=O)OC1Oc2cc(-c3c(C(N)=O)c(CC4CC4)nc4c3cnn4C3(c4ccc(F)cn4)CC3)ccc2N(Cc2ncc(F)cn2)C1=O] |
| 305 | 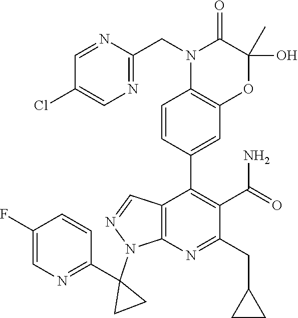 [SMILES: CC1(O)Oc2cc(-c3c(C(N)=O)c(CC4CC4)nc4c3cnn4C3(c4ccc(F)cn4)CC3)ccc2N(Cc2ncc(Cl)cn2)C1=O] |
| 306 | 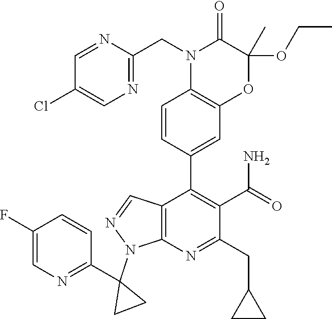 [SMILES: CCOC1(C)Oc2cc(-c3c(C(N)=O)c(CC4CC4)nc4c3cnn4C3(c4ccc(F)cn4)CC3)ccc2N(Cc2ncc(Cl)cn2)C1=O] |
| 307 | 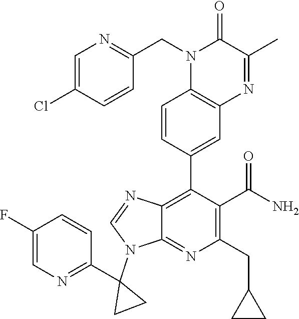 [SMILES: Cc1nc2cc(-c3c(C(N)=O)c(CC4CC4)nc4c3ncn4C3(c4ccc(F)cn4)CC3)ccc2n(Cc2ccc(Cl)cn2)c1=O] |
| 308 | 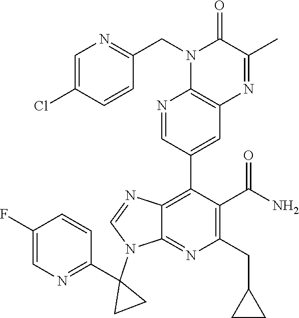 [SMILES: Cc1nc2cc(-c3c(C(N)=O)c(CC4CC4)nc4c3ncn4C3(c4ccc(F)cn4)CC3)cnc2n(Cc2ccc(Cl)cn2)c1=O] |
| 309 | 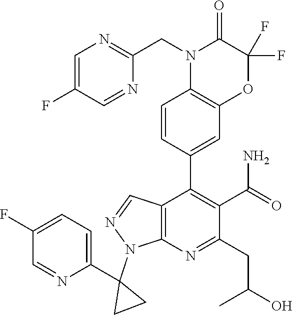 [SMILES: CC(O)Cc1nc2c(cnn2C2(c3ccc(F)cn3)CC2)c(-c2ccc3c(c2)OC(F)(F)C(=O)N3Cc2ncc(F)cn2)c1C(N)=O] |
| 310 | 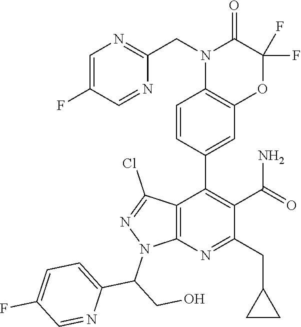 [SMILES: NC(=O)c1c(CC2CC2)nc2c(c(Cl)nn2C(CO)c2ccc(F)cn2)c1-c1ccc2c(c1)OC(F)(F)C(=O)N2Cc1ncc(F)cn1] |
| 311 | 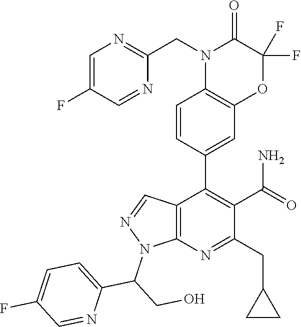 [SMILES: NC(=O)c1c(CC2CC2)nc2c(cnn2C(CO)c2ccc(F)cn2)c1-c1ccc2c(c1)OC(F)(F)C(=O)N2Cc1ncc(F)cn1] |
| 312 | 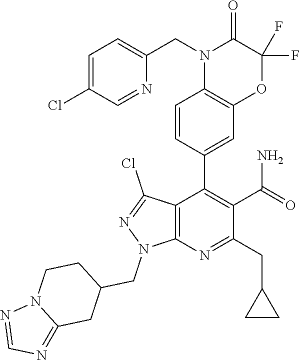 [SMILES: NC(=O)c1c(CC2CC2)nc2c(c(Cl)nn2CC2CCn3ncnc3C2)c1-c1ccc2c(c1)OC(F)(F)C(=O)N2Cc1ccc(Cl)cn1] |
| 313 | 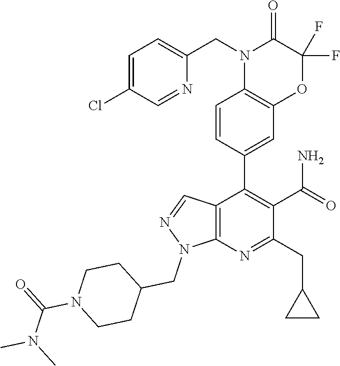 [SMILES: CN(C)C(=O)N1CCC(Cn2ncc3c(-c4ccc5c(c4)OC(F)(F)C(=O)N5Cc4ccc(Cl)cn4)c(C(N)=O)c(CC4CC4)nc32)CC1] |
| 314 | 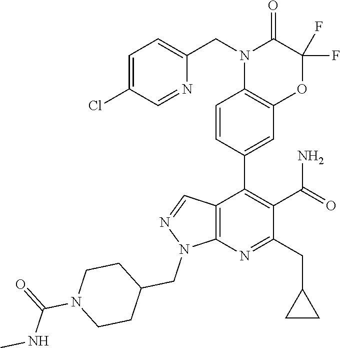 [SMILES: CNC(=O)N1CCC(Cn2ncc3c(-c4ccc5c(c4)OC(F)(F)C(=O)N5Cc4ccc(Cl)cn4)c(C(N)=O)c(CC4CC4)nc32)CC1] |
| 315 | 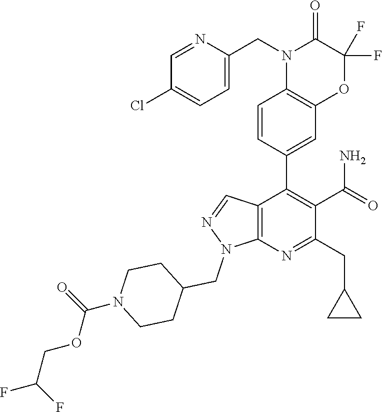 [SMILES: NC(=O)c1c(CC2CC2)nc2c(cnn2CC2CCN(C(=O)OCC(F)F)CC2)c1-c1ccc2c(c1)OC(F)(F)C(=O)N2Cc1ccc(Cl)cn1] |
| 316 | 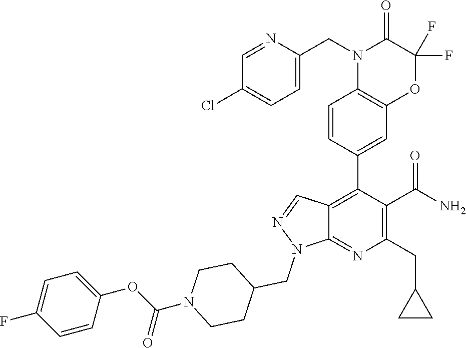 [SMILES: NC(=O)c1c(CC2CC2)nc2c(cnn2CC2CCN(C(=O)Oc3ccc(F)cc3)CC2)c1-c1ccc2c(c1)OC(F)(F)C(=O)N2Cc1ccc(Cl)cn1] |
| 317 | 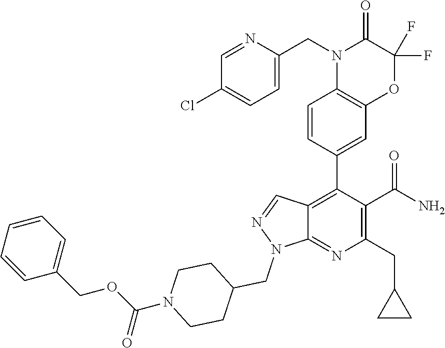 [SMILES: NC(=O)c1c(CC2CC2)nc2c(cnn2CC2CCN(C(=O)OCc3ccccc3)CC2)c1-c1ccc2c(c1)OC(F)(F)C(=O)N2Cc1ccc(Cl)cn1] |
| 318 | 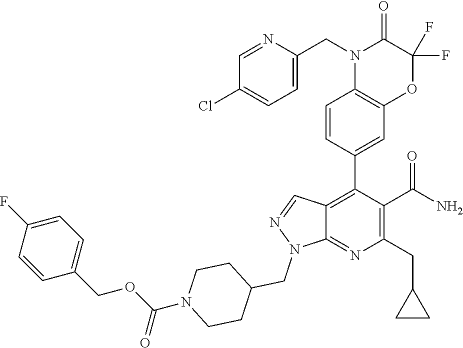 [SMILES: NC(=O)c1c(CC2CC2)nc2c(cnn2CC2CCN(C(=O)OCc3ccc(F)cc3)CC2)c1-c1ccc2c(c1)OC(F)(F)C(=O)N2Cc1ccc(Cl)cn1] |
| 319 | 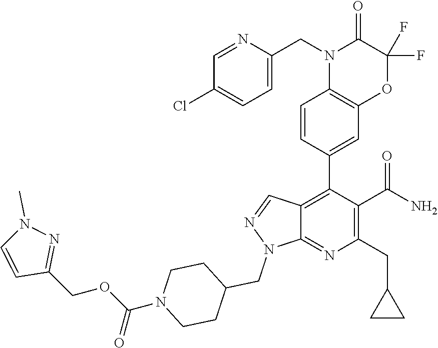 [SMILES: Cn1ccc(COC(=O)N2CCC(Cn3ncc4c(-c5ccc6c(c5)OC(F)(F)C(=O)N6Cc5ccc(Cl)cn5)c(C(N)=O)c(CC5CC5)nc43)CC2)n1] |
| 320 | 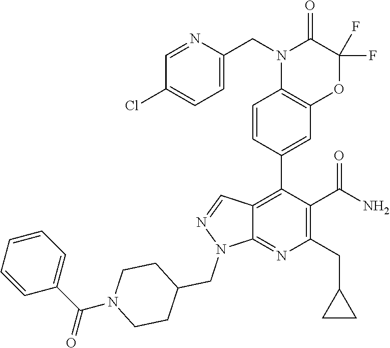 [SMILES: NC(=O)c1c(CC2CC2)nc2c(cnn2CC2CCN(C(=O)c3ccccc3)CC2)c1-c1ccc2c(c1)OC(F)(F)C(=O)N2Cc1ccc(Cl)cn1] |
| 321 | 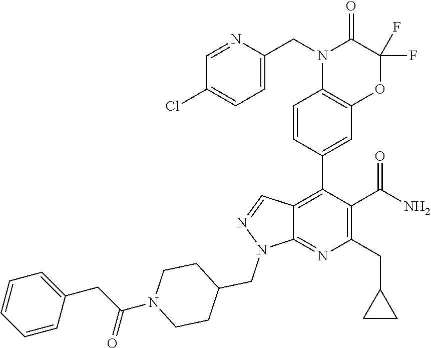 [SMILES: NC(=O)c1c(CC2CC2)nc2c(cnn2CC2CCN(C(=O)Cc3ccccc3)CC2)c1-c1ccc2c(c1)OC(F)(F)C(=O)N2Cc1ccc(Cl)cn1] |
| 322 | 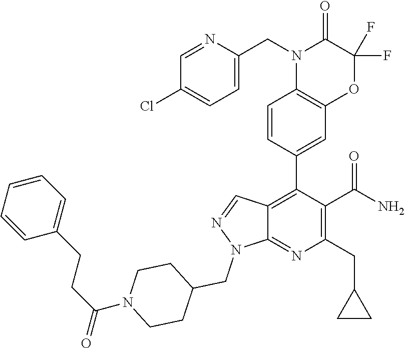 [SMILES: NC(=O)c1c(CC2CC2)nc2c(cnn2CC2CCN(C(=O)CCc3ccccc3)CC2)c1-c1ccc2c(c1)OC(F)(F)C(=O)N2Cc1ccc(Cl)cn1] |
| 323 | 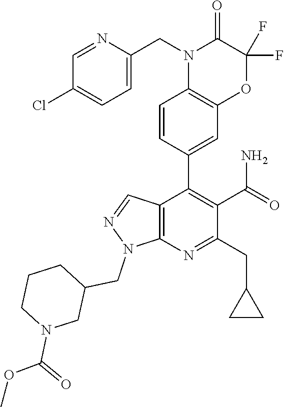 [SMILES: COC(=O)N1CCCC(Cn2ncc3c(-c4ccc5c(c4)OC(F)(F)C(=O)N5Cc4ccc(Cl)cn4)c(C(N)=O)c(CC4CC4)nc32)C1] |
| 324 | 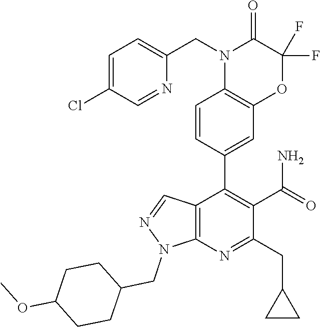 [SMILES: COC1CCC(Cn2ncc3c(-c4ccc5c(c4)OC(F)(F)C(=O)N5Cc4ccc(Cl)cn4)c(C(N)=O)c(CC4CC4)nc32)CC1] |
| 325 | 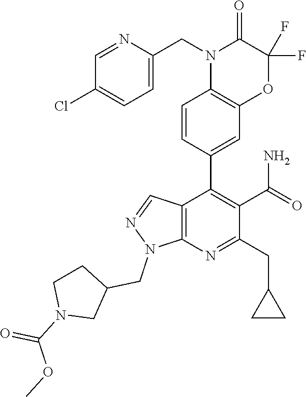 [SMILES: COC(=O)N1CCC(Cn2ncc3c(-c4ccc5c(c4)OC(F)(F)C(=O)N5Cc4ccc(Cl)cn4)c(C(N)=O)c(CC4CC4)nc32)C1] |
| 326 | 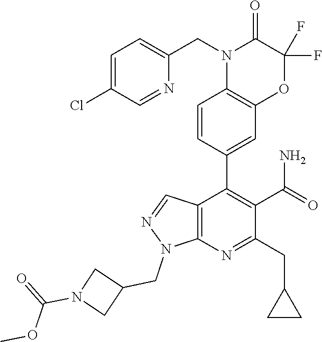 [SMILES: COC(=O)N1CC(Cn2ncc3c(-c4ccc5c(c4)OC(F)(F)C(=O)N5Cc4ccc(Cl)cn4)c(C(N)=O)c(CC4CC4)nc32)C1] |
| 327 | 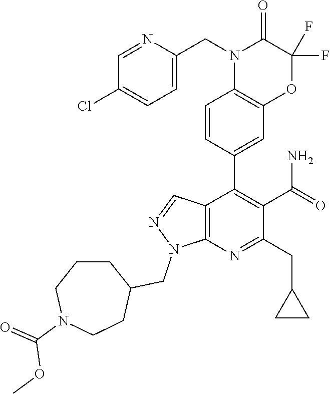 [SMILES: COC(=O)N1CCCC(Cn2ncc3c(-c4ccc5c(c4)OC(F)(F)C(=O)N5Cc4ccc(Cl)cn4)c(C(N)=O)c(CC4CC4)nc32)CC1] |
| 328 | 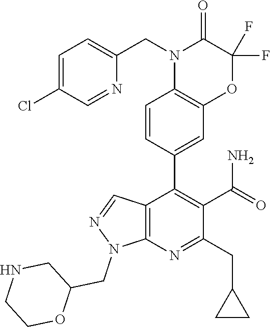 [SMILES: NC(=O)c1c(CC2CC2)nc2c(cnn2CC2CNCCO2)c1-c1ccc2c(c1)OC(F)(F)C(=O)N2Cc1ccc(Cl)cn1] |
| 329 | 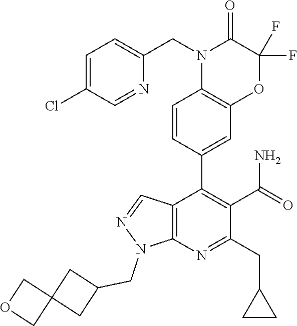 [SMILES: NC(=O)c1c(CC2CC2)nc2c(cnn2CC2CC3(COC3)C2)c1-c1ccc2c(c1)OC(F)(F)C(=O)N2Cc1ccc(Cl)cn1] |
| 330 | 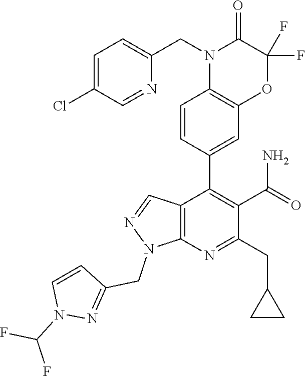 [SMILES: NC(=O)c1c(CC2CC2)nc2c(cnn2Cc2ccn(C(F)F)n2)c1-c1ccc2c(c1)OC(F)(F)C(=O)N2Cc1ccc(Cl)cn1] |
| 331 | 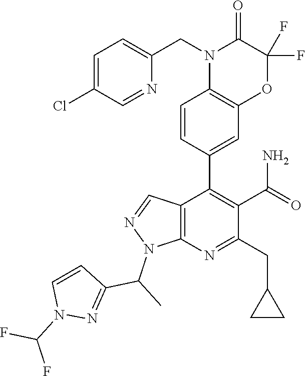 [SMILES: CC(c1ccn(C(F)F)n1)n1ncc2c(-c3ccc4c(c3)OC(F)(F)C(=O)N4Cc3ccc(Cl)cn3)c(C(N)=O)c(CC3CC3)nc21] |
| 332 | 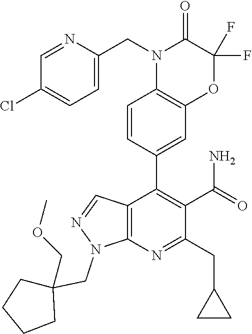 [SMILES: COCC1(Cn2ncc3c(-c4ccc5c(c4)OC(F)(F)C(=O)N5Cc4ccc(Cl)cn4)c(C(N)=O)c(CC4CC4)nc32)CCCC1] |
| 333 | 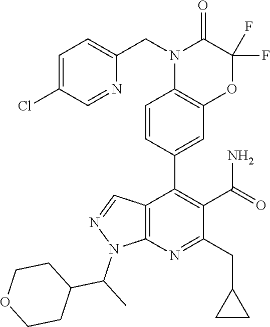 [SMILES: CC(C1CCOCC1)n1ncc2c(-c3ccc4c(c3)OC(F)(F)C(=O)N4Cc3ccc(Cl)cn3)c(C(N)=O)c(CC3CC3)nc21] |
| 334 | 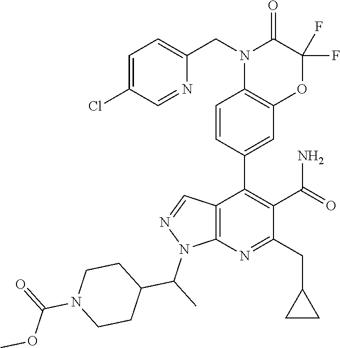 [SMILES: COC(=O)N1CCC(C(C)n2ncc3c(-c4ccc5c(c4)OC(F)(F)C(=O)N5Cc4ccc(Cl)cn4)c(C(N)=O)c(CC4CC4)nc32)CC1] |
| 335 | 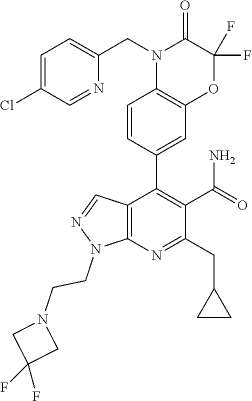 [SMILES: NC(=O)c1c(CC2CC2)nc2c(cnn2CCN2CC(F)(F)C2)c1-c1ccc2c(c1)OC(F)(F)C(=O)N2Cc1ccc(Cl)cn1] |
| 336 | 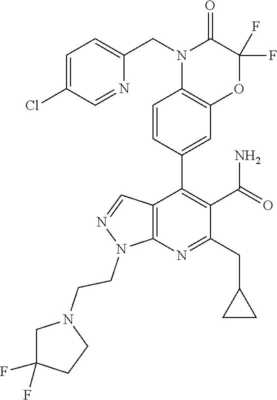 [SMILES: NC(=O)c1c(CC2CC2)nc2c(cnn2CCN2CCC(F)(F)C2)c1-c1ccc2c(c1)OC(F)(F)C(=O)N2Cc1ccc(Cl)cn1] |
| 337 | 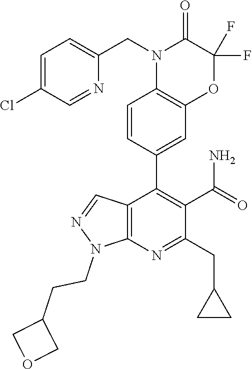 [SMILES: NC(=O)c1c(CC2CC2)nc2c(cnn2CCC2COC2)c1-c1ccc2c(c1)OC(F)(F)C(=O)N2Cc1ccc(Cl)cn1] |
| 338 | 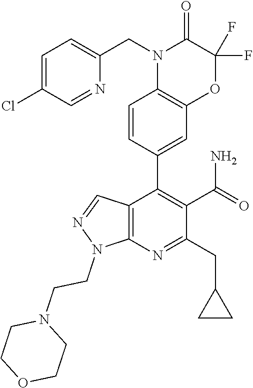 [SMILES: NC(=O)c1c(CC2CC2)nc2c(cnn2CCN2CCOCC2)c1-c1ccc2c(c1)OC(F)(F)C(=O)N2Cc1ccc(Cl)cn1] |
| 339 | 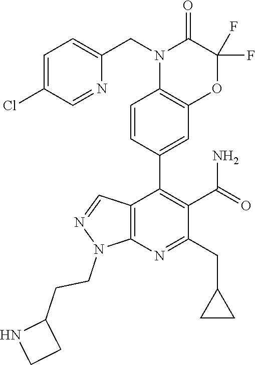 [SMILES: NC(=O)c1c(CC2CC2)nc2c(cnn2CCC2CCN2)c1-c1ccc2c(c1)OC(F)(F)C(=O)N2Cc1ccc(Cl)cn1] |
| 340 | 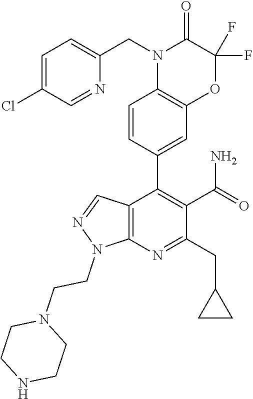 [SMILES: NC(=O)c1c(CC2CC2)nc2c(cnn2CCN2CCNCC2)c1-c1ccc2c(c1)OC(F)(F)C(=O)N2Cc1ccc(Cl)cn1] |
| 341 | 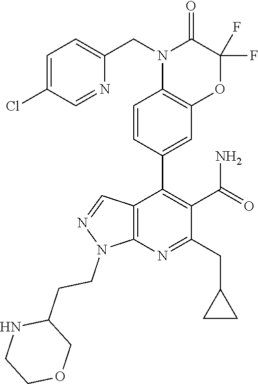 [SMILES: NC(=O)c1c(CC2CC2)nc2c(cnn2CCC2COCCN2)c1-c1ccc2c(c1)OC(F)(F)C(=O)N2Cc1ccc(Cl)cn1] |
| 342 | 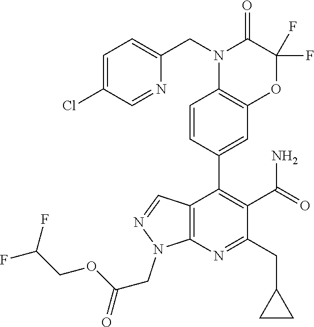 [SMILES: NC(=O)c1c(CC2CC2)nc2c(cnn2CC(=O)OCC(F)F)c1-c1ccc2c(c1)OC(F)(F)C(=O)N2Cc1ccc(Cl)cn1] |
| 343 | 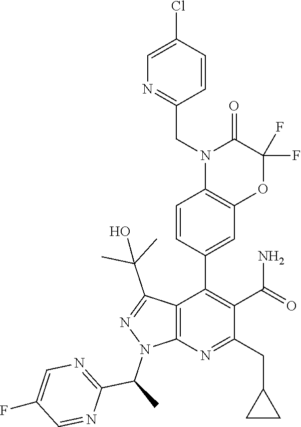 [SMILES: C[C@@H](c1ncc(F)cn1)n1nc(C(C)(C)O)c2c(-c3ccc4c(c3)OC(F)(F)C(=O)N4Cc3ccc(Cl)cn3)c(C(N)=O)c(CC3CC3)nc21] |
| 344 | 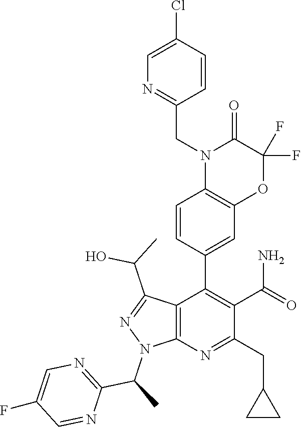 [SMILES: CC(O)c1nn([C@@H](C)c2ncc(F)cn2)c2nc(CC3CC3)c(C(N)=O)c(-c3ccc4c(c3)OC(F)(F)C(=O)N4Cc3ccc(Cl)cn3)c12] |
| 345 | 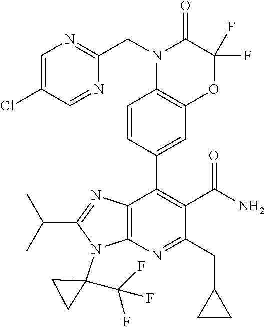 [SMILES: CC(C)c1nc2c(-c3ccc4c(c3)OC(F)(F)C(=O)N4Cc3ncc(Cl)cn3)c(C(N)=O)c(CC3CC3)nc2n1C1(C(F)(F)F)CC1] |
| 346 | 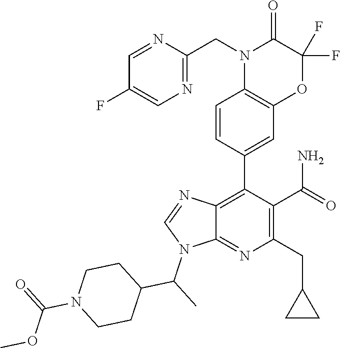 [SMILES: COC(=O)N1CCC(C(C)n2cnc3c(-c4ccc5c(c4)OC(F)(F)C(=O)N5Cc4ncc(F)cn4)c(C(N)=O)c(CC4CC4)nc32)CC1] |
| 347 | 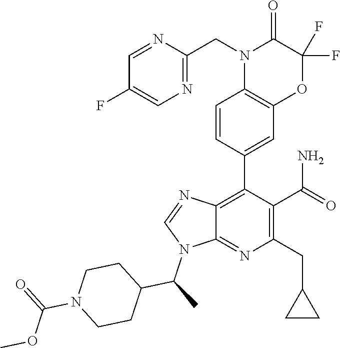 [SMILES: COC(=O)N1CCC([C@H](C)n2cnc3c(-c4ccc5c(c4)OC(F)(F)C(=O)N5Cc4ncc(F)cn4)c(C(N)=O)c(CC4CC4)nc32)CC1] |
| 348 | 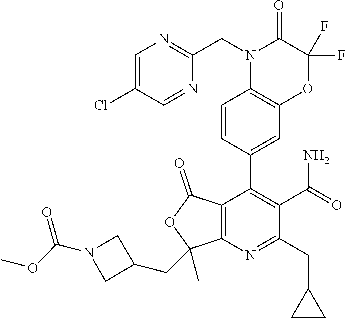 [SMILES: COC(=O)N1CC(CC2(C)OC(=O)c3c2nc(CC2CC2)c(C(N)=O)c3-c2ccc3c(c2)OC(F)(F)C(=O)N3Cc2ncc(Cl)cn2)C1] |
| 349 | 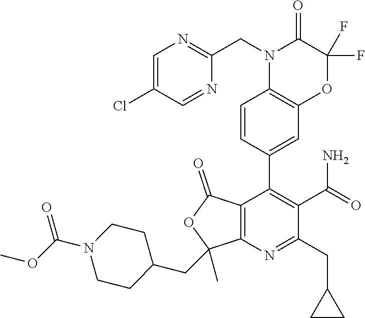 [SMILES: COC(=O)N1CCC(CC2(C)OC(=O)c3c2nc(CC2CC2)c(C(N)=O)c3-c2ccc3c(c2)OC(F)(F)C(=O)N3Cc2ncc(Cl)cn2)CC1] |
| 350 | 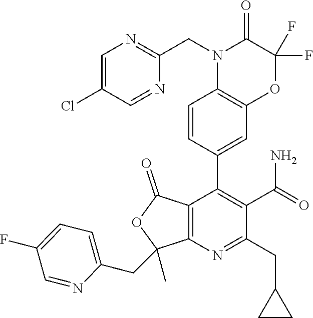 [SMILES: CC1(Cc2ccc(F)cn2)OC(=O)c2c1nc(CC1CC1)c(C(N)=O)c2-c1ccc2c(c1)OC(F)(F)C(=O)N2Cc1ncc(Cl)cn1] |
| 351 | 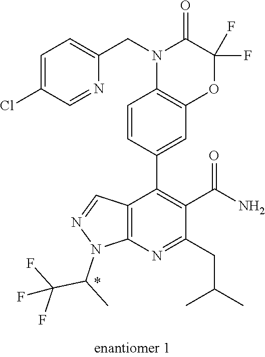 [SMILES: CC(C)Cc1nc2c(cnn2C(C)C(F)(F)F)c(-c2ccc3c(c2)OC(F)(F)C(=O)N3Cc2ccc(Cl)cn2)c1C(N)=O] |
| 352 | 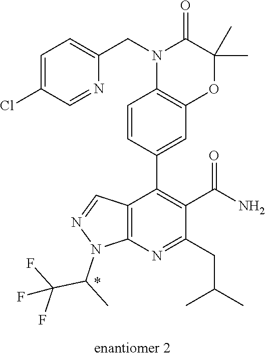 [SMILES: CC(C)Cc1nc2c(cnn2C(C)C(F)(F)F)c(-c2ccc3c(c2)OC(C)(C)C(=O)N3Cc2ccc(Cl)cn2)c1C(N)=O] |
| 353 | 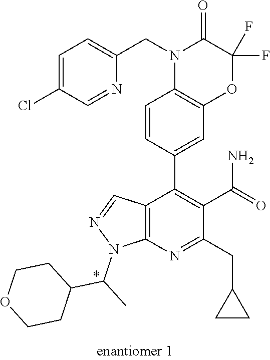 [SMILES: CC(C1CCOCC1)n1ncc2c(-c3ccc4c(c3)OC(F)(F)C(=O)N4Cc3ccc(Cl)cn3)c(C(N)=O)c(CC3CC3)nc21] |
| 354 | 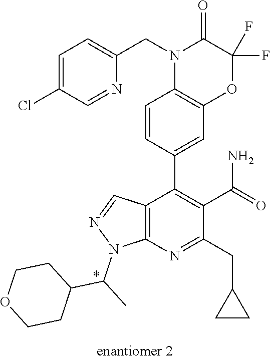 [SMILES: CC(C1CCOCC1)n1ncc2c(-c3ccc4c(c3)OC(F)(F)C(=O)N4Cc3ccc(Cl)cn3)c(C(N)=O)c(CC3CC3)nc21] |
| 355 | 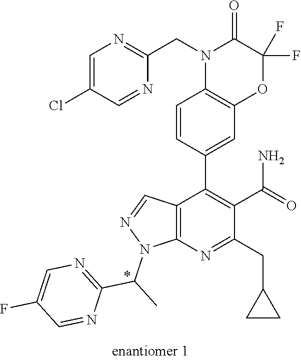 [SMILES: CC(c1ncc(F)cn1)n1ncc2c(-c3ccc4c(c3)OC(F)(F)C(=O)N4Cc3ncc(Cl)cn3)c(C(N)=O)c(CC3CC3)nc21] |
| 356 | 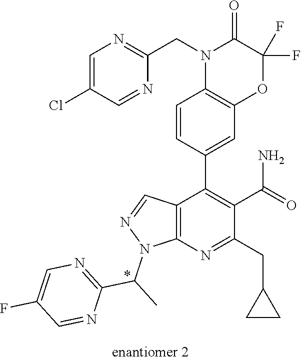 [SMILES: CC(c1ncc(F)cn1)n1ncc2c(-c3ccc4c(c3)OC(F)(F)C(=O)N4Cc3ncc(Cl)cn3)c(C(N)=O)c(CC3CC3)nc21] |
| 357 | 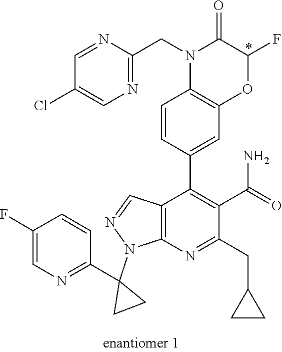 [SMILES: NC(=O)c1c(CC2CC2)nc2c(cnn2C2(c3ccc(F)cn3)CC2)c1-c1ccc2c(c1)OC(F)C(=O)N2Cc1ncc(Cl)cn1] |
| 358 | 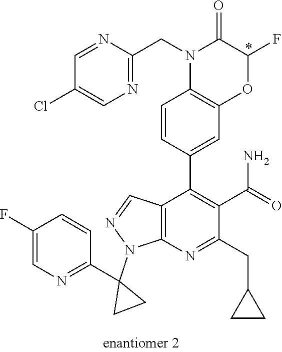 [SMILES: NC(=O)c1c(CC2CC2)nc2c(cnn2C2(c3ccc(F)cn3)CC2)c1-c1ccc2c(c1)OC(F)C(=O)N2Cc1ncc(Cl)cn1] |
| 359 | 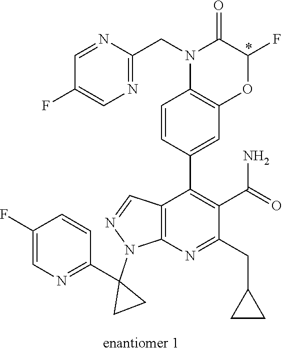 [SMILES: NC(=O)c1c(CC2CC2)nc2c(cnn2C2(c3ccc(F)cn3)CC2)c1-c1ccc2c(c1)OC(F)C(=O)N2Cc1ncc(F)cn1] |
| 360 | 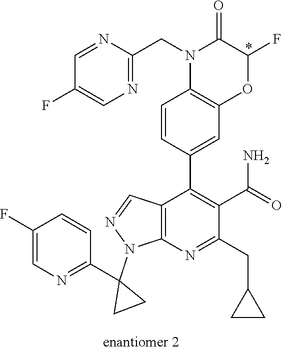 [SMILES: NC(=O)c1c(CC2CC2)nc2c(cnn2C2(c3ccc(F)cn3)CC2)c1-c1ccc2c(c1)OC(F)C(=O)N2Cc1ncc(F)cn1] |
| 361 | 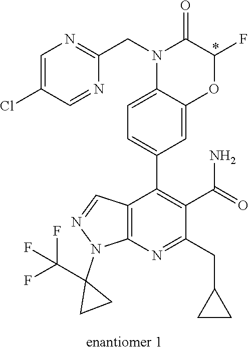 [SMILES: NC(=O)c1c(CC2CC2)nc2c(cnn2C2(C(F)(F)F)CC2)c1-c1ccc2c(c1)OC(F)C(=O)N2Cc1ncc(Cl)cn1] |
| 362 | 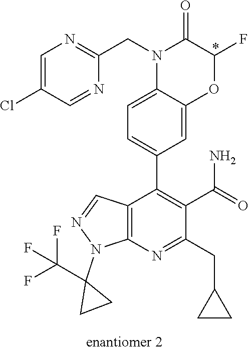 [SMILES: NC(=O)c1c(CC2CC2)nc2c(cnn2C2(C(F)(F)F)CC2)c1-c1ccc2c(c1)OC(F)C(=O)N2Cc1ncc(Cl)cn1] |
| 363 | 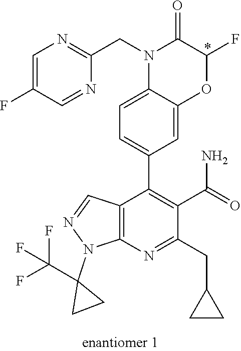 [SMILES: NC(=O)c1c(CC2CC2)nc2c(cnn2C2(C(F)(F)F)CC2)c1-c1ccc2c(c1)OC(F)C(=O)N2Cc1ncc(F)cn1] |
| 364 | 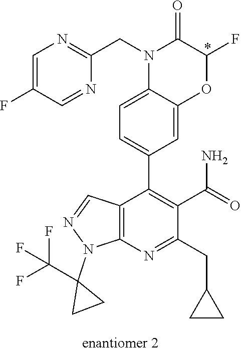 [SMILES: NC(=O)c1c(CC2CC2)nc2c(cnn2C2(C(F)(F)F)CC2)c1-c1ccc2c(c1)OC(F)C(=O)N2Cc1ncc(F)cn1] |

The compounds of Formula I, II, III, IV, V, VI, VII, and VIII, and subformulas provided herein encompass stereochemical forms of the compounds, for example, optical isomers, such as enantiomers, diastereomers, as well as mixtures thereof, e.g., mixtures of enantiomers and/or diastereomers, including racemic mixtures, as well as equal or non-equal mixtures of individual enantiomers and/or diastereomers. All stereochemical forms are contemplated in this disclosure. Unless otherwise indicated, when a disclosed compound is named or depicted by a structure without specifying the stereochemistry and has one or more chiral centers, it is understood to represent all possible stereoisomers of the compound. Representative stereochemical forms are provided throughout the specification, including but not limited to those delineated in Table 2. In some embodiments, provided is compound selected from Table 2, or a pharmaceutically acceptable salt, stereoisomer, mixture of stereoisomers, or solvate thereof:

TABLE 2
Structure

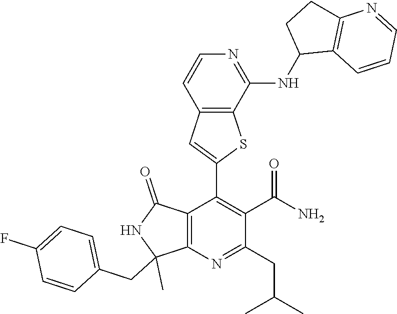 [SMILES: CC(C)Cc1nc2c(c(-c3cc4ccnc(NC5CCc6ncccc65)c4s3)c1C(N)=O)C(=O)NC2(C)Cc1ccc(F)cc1]

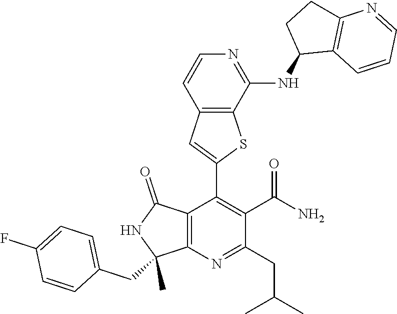 [SMILES: CC(C)Cc1nc2c(c(-c3cc4ccnc(N[C@H]5CCc6ncccc65)c4s3)c1C(N)=O)C(=O)N[C@@]2(C)Cc1ccc(F)cc1]

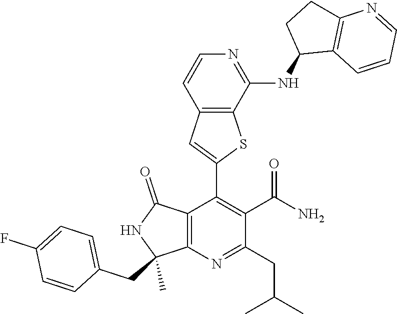 [SMILES: CC(C)Cc1nc2c(c(-c3cc4ccnc(N[C@H]5CCc6ncccc65)c4s3)c1C(N)=O)C(=O)N[C@]2(C)Cc1ccc(F)cc1]

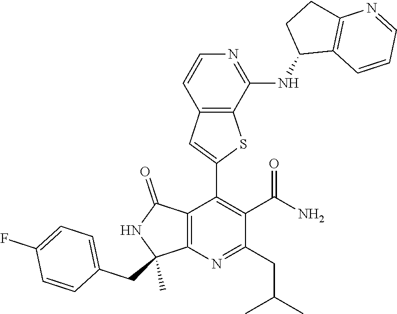 [SMILES: CC(C)Cc1nc2c(c(-c3cc4ccnc(N[C@@H]5CCc6ncccc65)c4s3)c1C(N)=O)C(=O)N[C@]2(C)Cc1ccc(F)cc1]

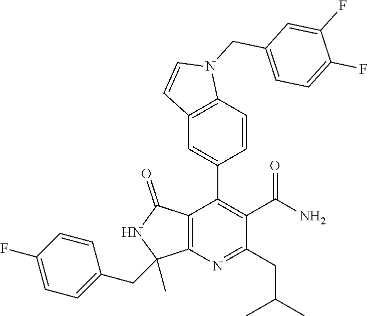 [SMILES: CC(C)Cc1nc2c(c(-c3ccc4c(ccn4Cc4ccc(F)c(F)c4)c3)c1C(N)=O)C(=O)NC2(C)Cc1ccc(F)cc1]

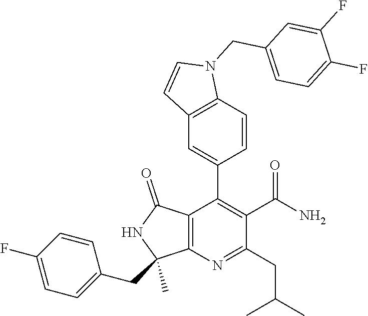 [SMILES: CC(C)Cc1nc2c(c(-c3ccc4c(ccn4Cc4ccc(F)c(F)c4)c3)c1C(N)=O)C(=O)N[C@]2(C)Cc1ccc(F)cc1]

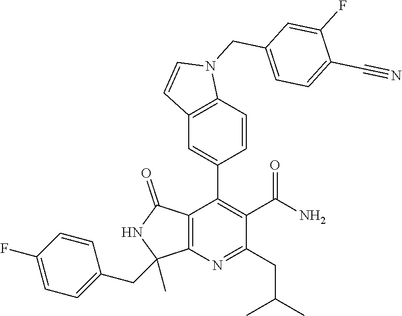 [SMILES: CC(C)Cc1nc2c(c(-c3ccc4c(ccn4Cc4ccc(C#N)c(F)c4)c3)c1C(N)=O)C(=O)NC2(C)Cc1ccc(F)cc1]

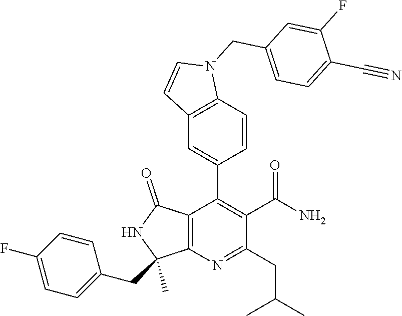 [SMILES: CC(C)Cc1nc2c(c(-c3ccc4c(ccn4Cc4ccc(C#N)c(F)c4)c3)c1C(N)=O)C(=O)N[C@]2(C)Cc1ccc(F)cc1]

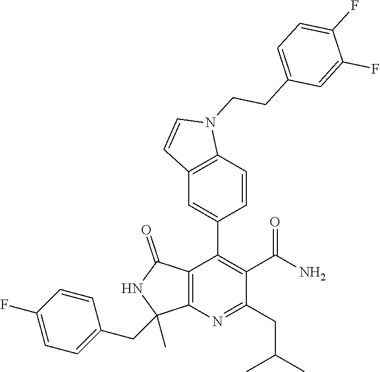 [SMILES: CC(C)Cc1nc2c(c(-c3ccc4c(ccn4CCc4ccc(F)c(F)c4)c3)c1C(N)=O)C(=O)NC2(C)Cc1ccc(F)cc1]

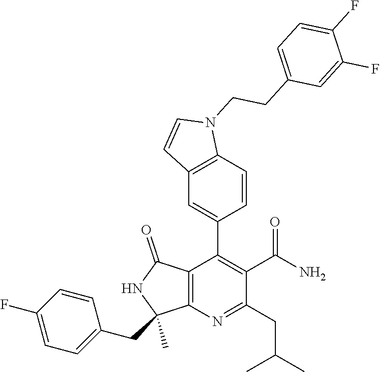 [SMILES: CC(C)Cc1nc2c(c(-c3ccc4c(ccn4CCc4ccc(F)c(F)c4)c3)c1C(N)=O)C(=O)N[C@]2(C)Cc1ccc(F)cc1]

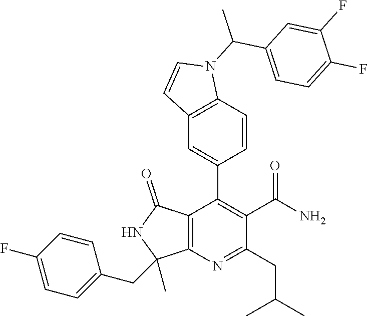 [SMILES: CC(C)Cc1nc2c(c(-c3ccc4c(ccn4C(C)c4ccc(F)c(F)c4)c3)c1C(N)=O)C(=O)NC2(C)Cc1ccc(F)cc1]

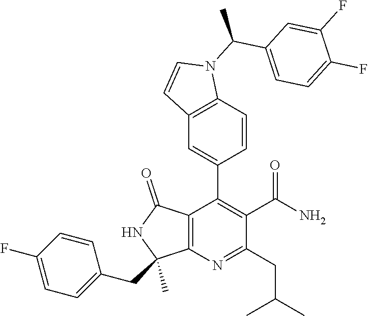 [SMILES: CC(C)Cc1nc2c(c(-c3ccc4c(ccn4[C@@H](C)c4ccc(F)c(F)c4)c3)c1C(N)=O)C(=O)N[C@]2(C)Cc1ccc(F)cc1]

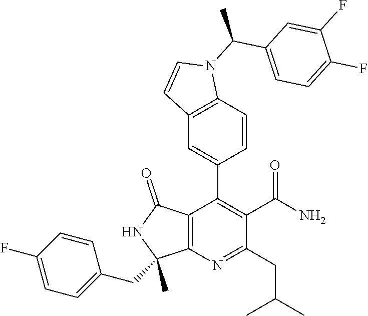 [SMILES: CC(C)Cc1nc2c(c(-c3ccc4c(ccn4[C@@H](C)c4ccc(F)c(F)c4)c3)c1C(N)=O)C(=O)N[C@@]2(C)Cc1ccc(F)cc1]

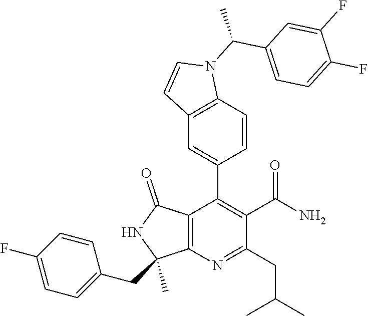 [SMILES: CC(C)Cc1nc2c(c(-c3ccc4c(ccn4[C@H](C)c4ccc(F)c(F)c4)c3)c1C(N)=O)C(=O)N[C@]2(C)Cc1ccc(F)cc1]

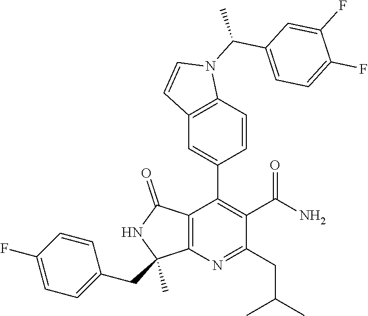 [SMILES: CC(C)Cc1nc2c(c(-c3ccc4c(ccn4[C@H](C)c4ccc(F)c(F)c4)c3)c1C(N)=O)C(=O)N[C@]2(C)Cc1ccc(F)cc1]

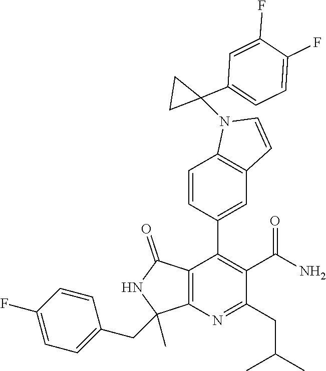 [SMILES: CC(C)Cc1nc2c(c(-c3ccc4c(ccn4C4(c5ccc(F)c(F)c5)CC4)c3)c1C(N)=O)C(=O)NC2(C)Cc1ccc(F)cc1]

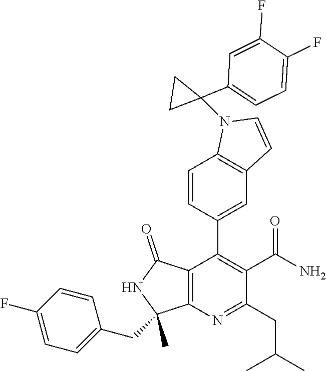 [SMILES: CC(C)Cc1nc2c(c(-c3ccc4c(ccn4C4(c5ccc(F)c(F)c5)CC4)c3)c1C(N)=O)C(=O)N[C@@]2(C)Cc1ccc(F)cc1]

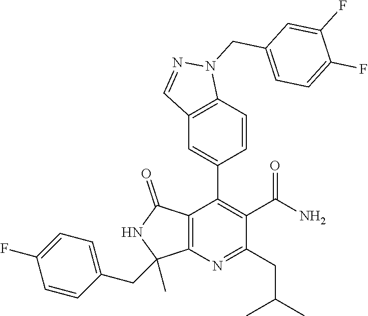 [SMILES: CC(C)Cc1nc2c(c(-c3ccc4c(cnn4Cc4ccc(F)c(F)c4)c3)c1C(N)=O)C(=O)NC2(C)Cc1ccc(F)cc1]

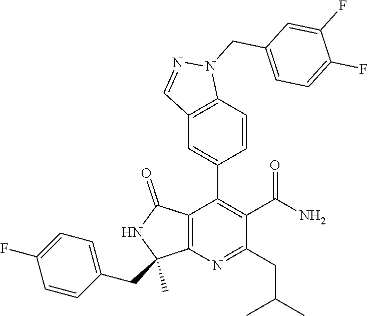 [SMILES: CC(C)Cc1nc2c(c(-c3ccc4c(cnn4Cc4ccc(F)c(F)c4)c3)c1C(N)=O)C(=O)N[C@]2(C)Cc1ccc(F)cc1]

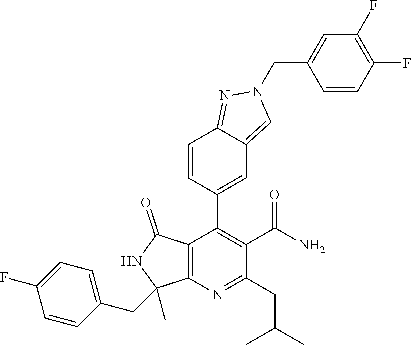 [SMILES: CC(C)Cc1nc2c(c(-c3ccc4nn(Cc5ccc(F)c(F)c5)cc4c3)c1C(N)=O)C(=O)NC2(C)Cc1ccc(F)cc1]

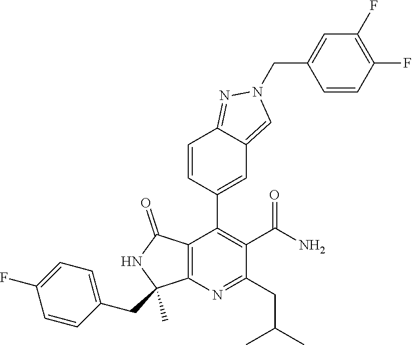 [SMILES: CC(C)Cc1nc2c(c(-c3ccc4nn(Cc5ccc(F)c(F)c5)cc4c3)c1C(N)=O)C(=O)N[C@]2(C)Cc1ccc(F)cc1]

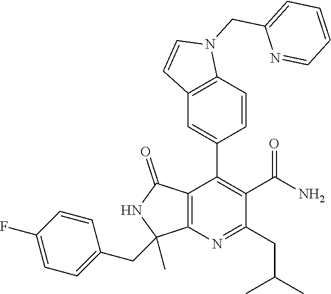 [SMILES: CC(C)Cc1nc2c(c(-c3ccc4c(ccn4Cc4ccccn4)c3)c1C(N)=O)C(=O)NC2(C)Cc1ccc(F)cc1]

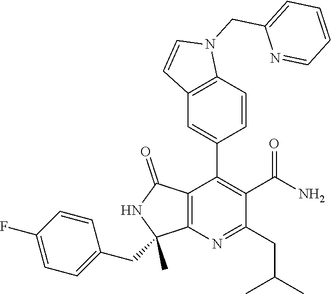 [SMILES: CC(C)Cc1nc2c(c(-c3ccc4c(ccn4Cc4ccccn4)c3)c1C(N)=O)C(=O)N[C@@]2(C)Cc1ccc(F)cc1]

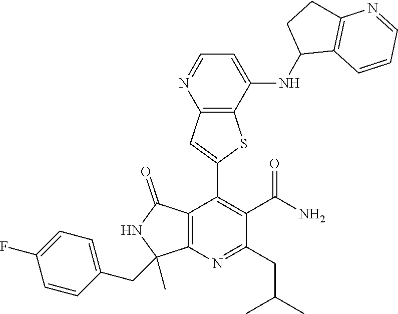 [SMILES: CC(C)Cc1nc2c(c(-c3cc4nccc(NC5CCc6ncccc65)c4s3)c1C(N)=O)C(=O)NC2(C)Cc1ccc(F)cc1]

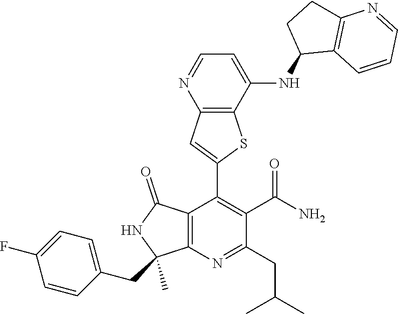 [SMILES: CC(C)Cc1nc2c(c(-c3cc4nccc(N[C@H]5CCc6ncccc65)c4s3)c1C(N)=O)C(=O)N[C@]2(C)Cc1ccc(F)cc1]

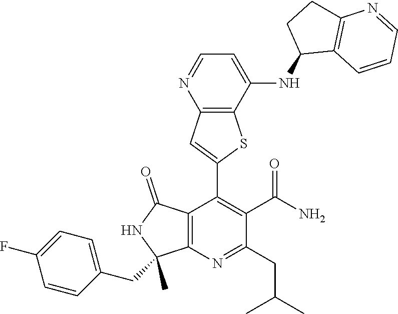 [SMILES: CC(C)Cc1nc2c(c(-c3cc4nccc(N[C@H]5CCc6ncccc65)c4s3)c1C(N)=O)C(=O)N[C@@]2(C)Cc1ccc(F)cc1]

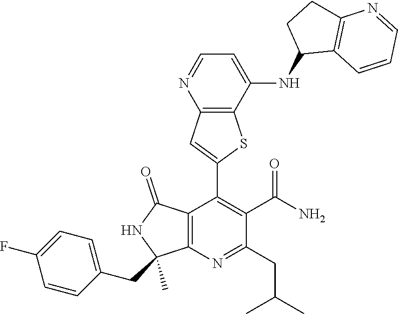

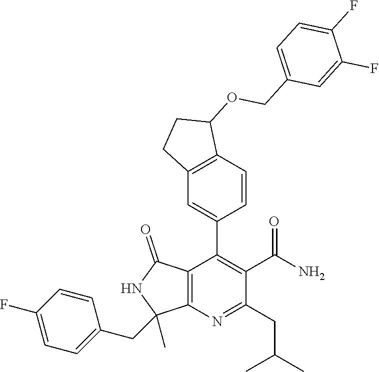 [SMILES: CC(C)Cc1nc2c(c(-c3ccc4c(c3)CCC4OCc3ccc(F)c(F)c3)c1C(N)=O)C(=O)NC2(C)Cc1ccc(F)cc1]

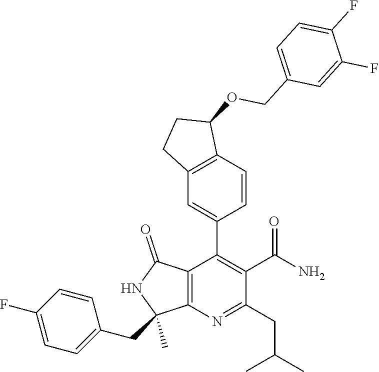 [SMILES: CC(C)Cc1nc2c(c(-c3ccc4c(c3)CC[C@H]4OCc3ccc(F)c(F)c3)c1C(N)=O)C(=O)N[C@]2(C)Cc1ccc(F)cc1]

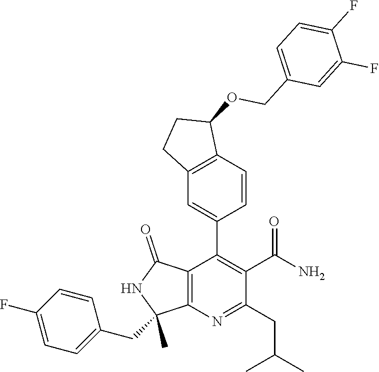 [SMILES: CC(C)Cc1nc2c(c(-c3ccc4c(c3)CC[C@H]4OCc3ccc(F)c(F)c3)c1C(N)=O)C(=O)N[C@@]2(C)Cc1ccc(F)cc1]

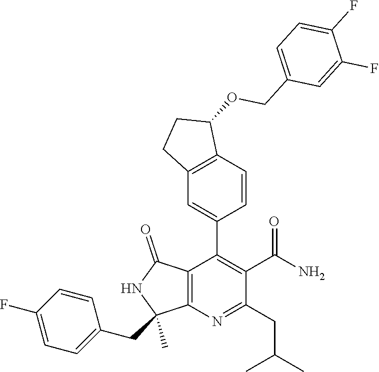 [SMILES: CC(C)Cc1nc2c(c(-c3ccc4c(c3)CC[C@@H]4OCc3ccc(F)c(F)c3)c1C(N)=O)C(=O)N[C@]2(C)Cc1ccc(F)cc1]

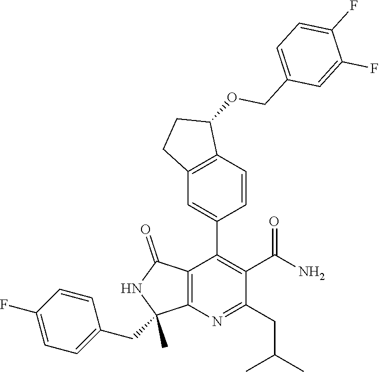 [SMILES: CC(C)Cc1nc2c(c(-c3ccc4c(c3)CC[C@@H]4OCc3ccc(F)c(F)c3)c1C(N)=O)C(=O)N[C@@]2(C)Cc1ccc(F)cc1]

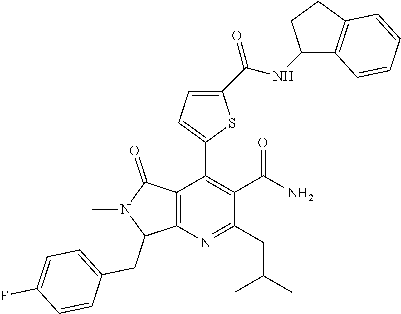 [SMILES: CC(C)Cc1nc2c(c(-c3ccc(C(=O)NC4CCc5ccccc54)s3)c1C(N)=O)C(=O)N(C)C2Cc1ccc(F)cc1]

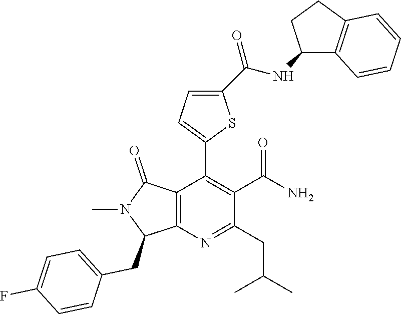 [SMILES: CC(C)Cc1nc2c(c(-c3ccc(C(=O)N[C@H]4CCc5ccccc54)s3)c1C(N)=O)C(=O)N(C)[C@@H]2Cc1ccc(F)cc1]

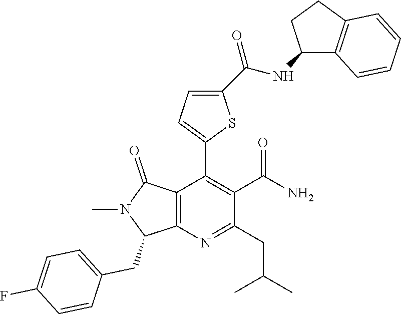 [SMILES: CC(C)Cc1nc2c(c(-c3ccc(C(=O)N[C@H]4CCc5ccccc54)s3)c1C(N)=O)C(=O)N(C)[C@H]2Cc1ccc(F)cc1]

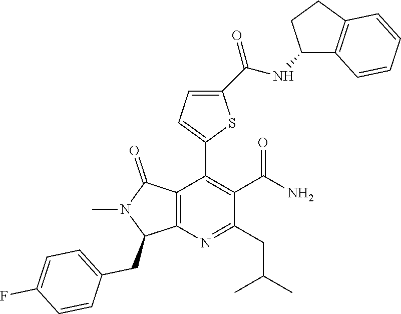 [SMILES: CC(C)Cc1nc2c(c(-c3ccc(C(=O)N[C@@H]4CCc5ccccc54)s3)c1C(N)=O)C(=O)N(C)[C@@H]2Cc1ccc(F)cc1]

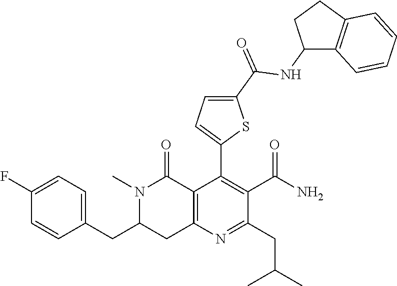 [SMILES: CC(C)Cc1nc2c(c(-c3ccc(C(=O)NC4CCc5ccccc54)s3)c1C(N)=O)C(=O)N(C)C(Cc1ccc(F)cc1)C2]

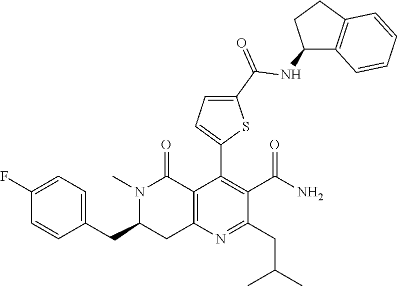 [SMILES: CC(C)Cc1nc2c(c(-c3ccc(C(=O)N[C@H]4CCc5ccccc54)s3)c1C(N)=O)C(=O)N(C)[C@H](Cc1ccc(F)cc1)C2]

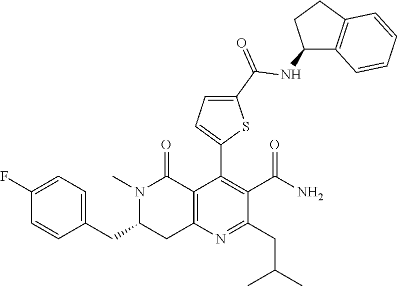 [SMILES: CC(C)Cc1nc2c(c(-c3ccc(C(=O)N[C@H]4CCc5ccccc54)s3)c1C(N)=O)C(=O)N(C)[C@@H](Cc1ccc(F)cc1)C2]

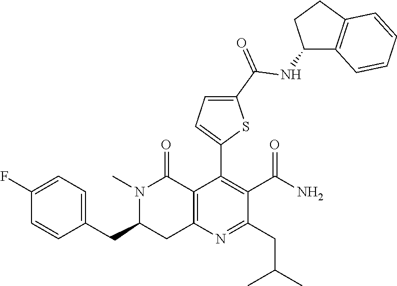 [SMILES: CC(C)Cc1nc2c(c(-c3ccc(C(=O)N[C@@H]4CCc5ccccc54)s3)c1C(N)=O)C(=O)N(C)[C@H](Cc1ccc(F)cc1)C2]

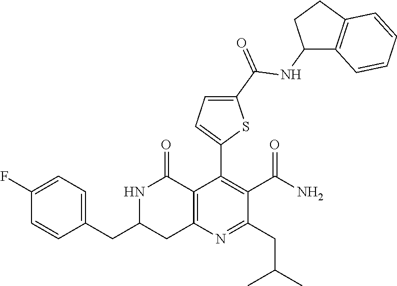 [SMILES: CC(C)Cc1nc2c(c(-c3ccc(C(=O)NC4CCc5ccccc54)s3)c1C(N)=O)C(=O)NC(Cc1ccc(F)cc1)C2]

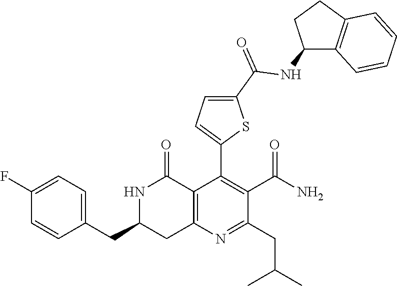 [SMILES: CC(C)Cc1nc2c(c(-c3ccc(C(=O)N[C@H]4CCc5ccccc54)s3)c1C(N)=O)C(=O)N[C@H](Cc1ccc(F)cc1)C2]

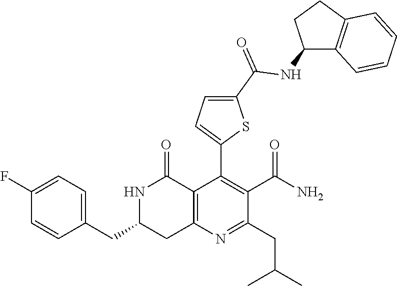 [SMILES: CC(C)Cc1nc2c(c(-c3ccc(C(=O)N[C@H]4CCc5ccccc54)s3)c1C(N)=O)C(=O)N[C@@H](Cc1ccc(F)cc1)C2]

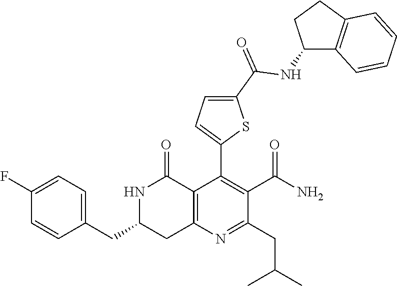 [SMILES: CC(C)Cc1nc2c(c(-c3ccc(C(=O)N[C@@H]4CCc5ccccc54)s3)c1C(N)=O)C(=O)N[C@@H](Cc1ccc(F)cc1)C2]

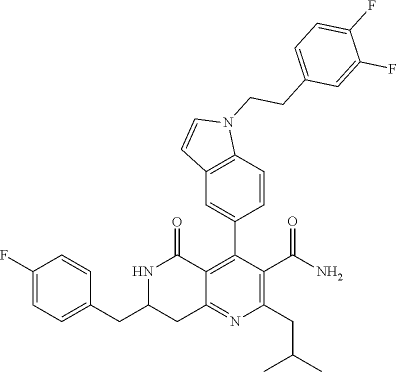 [SMILES: CC(C)Cc1nc2c(c(-c3ccc4c(ccn4CCc4ccc(F)c(F)c4)c3)c1C(N)=O)C(=O)NC(Cc1ccc(F)cc1)C2]

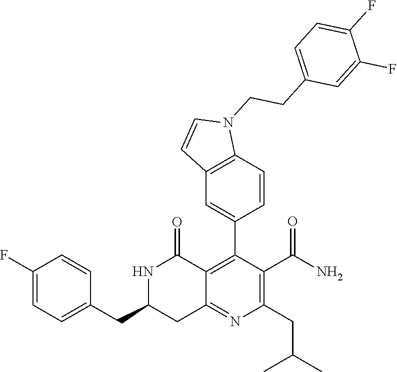 [SMILES: CC(C)Cc1nc2c(c(-c3ccc4c(ccn4CCc4ccc(F)c(F)c4)c3)c1C(N)=O)C(=O)N[C@H](Cc1ccc(F)cc1)C2]

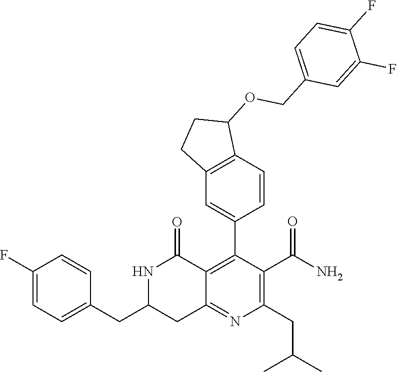 [SMILES: CC(C)Cc1nc2c(c(-c3ccc4c(c3)CCC4OCc3ccc(F)c(F)c3)c1C(N)=O)C(=O)NC(Cc1ccc(F)cc1)C2]

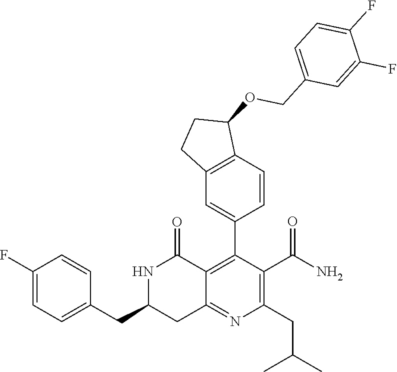 [SMILES: CC(C)Cc1nc2c(c(-c3ccc4c(c3)CC[C@H]4OCc3ccc(F)c(F)c3)c1C(N)=O)C(=O)N[C@H](Cc1ccc(F)cc1)C2]

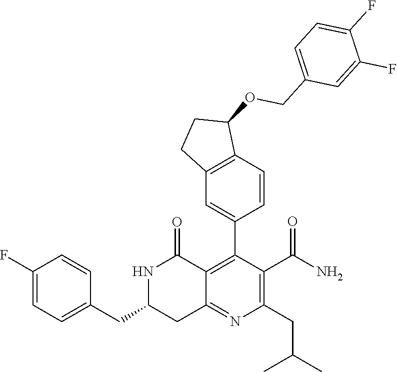 [SMILES: CC(C)Cc1nc2c(c(-c3ccc4c(c3)CC[C@H]4OCc3ccc(F)c(F)c3)c1C(N)=O)C(=O)N[C@@H](Cc1ccc(F)cc1)C2]

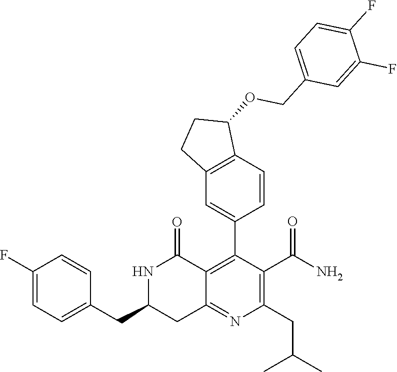 [SMILES: CC(C)Cc1nc2c(c(-c3ccc4c(c3)CC[C@@H]4OCc3ccc(F)c(F)c3)c1C(N)=O)C(=O)N[C@H](Cc1ccc(F)cc1)C2]

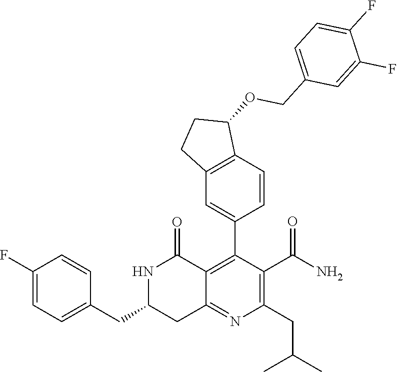 [SMILES: CC(C)Cc1nc2c(c(-c3ccc4c(c3)CC[C@@H]4OCc3ccc(F)c(F)c3)c1C(N)=O)C(=O)N[C@@H](Cc1ccc(F)cc1)C2]

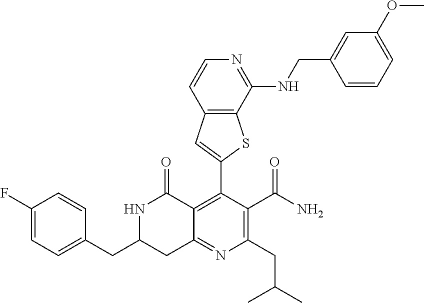 [SMILES: COc1cccc(CNc2nccc3cc(-c4c(C(N)=O)c(CC(C)C)nc5c4C(=O)NC(Cc4ccc(F)cc4)C5)sc23)c1]

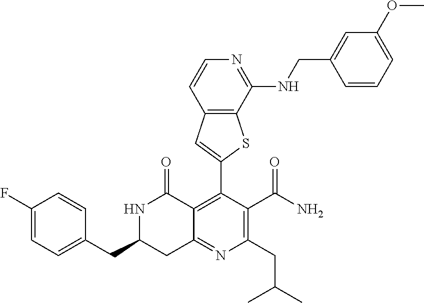 [SMILES: COc1cccc(CNc2nccc3cc(-c4c(C(N)=O)c(CC(C)C)nc5c4C(=O)N[C@H](Cc4ccc(F)cc4)C5)sc23)c1]

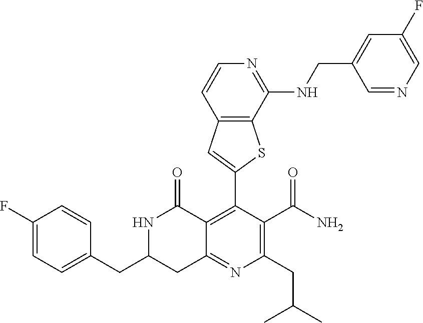 [SMILES: CC(C)Cc1nc2c(c(-c3cc4ccnc(NCc5cncc(F)c5)c4s3)c1C(N)=O)C(=O)NC(Cc1ccc(F)cc1)C2]

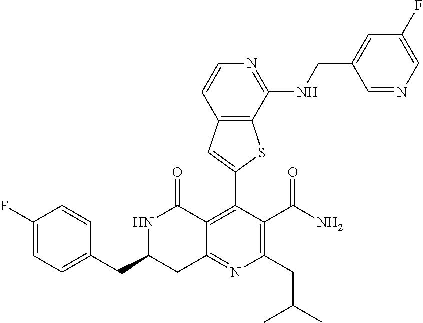 [SMILES: CC(C)Cc1nc2c(c(-c3cc4ccnc(NCc5cncc(F)c5)c4s3)c1C(N)=O)C(=O)N[C@H](Cc1ccc(F)cc1)C2]

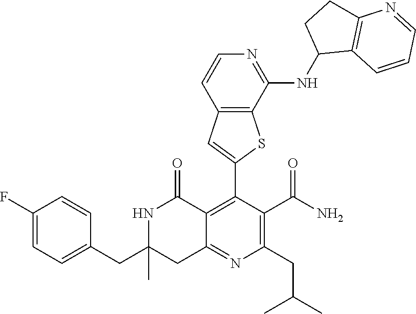 [SMILES: CC(C)Cc1nc2c(c(-c3cc4ccnc(NC5CCc6ncccc65)c4s3)c1C(N)=O)C(=O)NC(C)(Cc1ccc(F)cc1)C2]

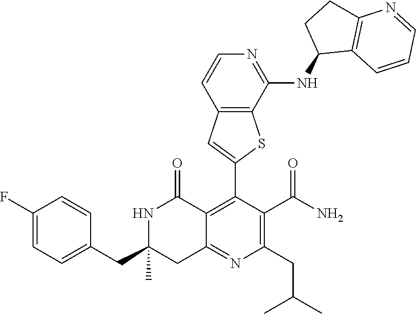 [SMILES: CC(C)Cc1nc2c(c(-c3cc4ccnc(N[C@H]5CCc6ncccc65)c4s3)c1C(N)=O)C(=O)N[C@](C)(Cc1ccc(F)cc1)C2]

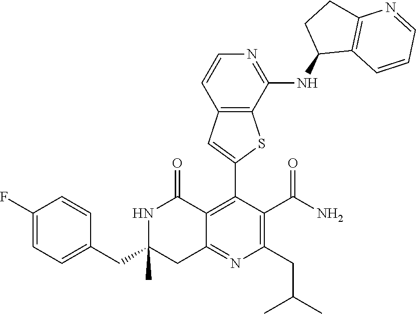 [SMILES: CC(C)Cc1nc2c(c(-c3cc4ccnc(N[C@H]5CCc6ncccc65)c4s3)c1C(N)=O)C(=O)N[C@@](C)(Cc1ccc(F)cc1)C2]

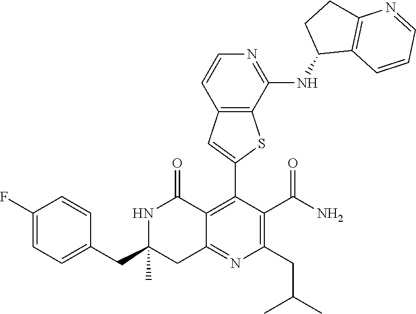 [SMILES: CC(C)Cc1nc2c(c(-c3cc4ccnc(N[C@@H]5CCc6ncccc65)c4s3)c1C(N)=O)C(=O)N[C@](C)(Cc1ccc(F)cc1)C2]

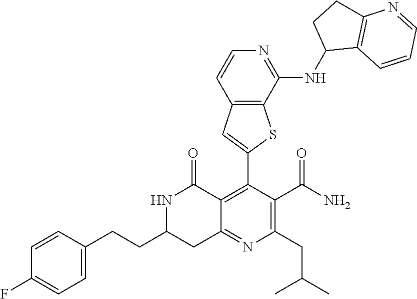 [SMILES: CC(C)Cc1nc2c(c(-c3cc4ccnc(NC5CCc6ncccc65)c4s3)c1C(N)=O)C(=O)NC(CCc1ccc(F)cc1)C2]

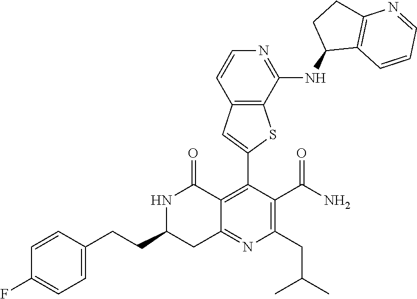 [SMILES: CC(C)Cc1nc2c(c(-c3cc4ccnc(N[C@H]5CCc6ncccc65)c4s3)c1C(N)=O)C(=O)N[C@H](CCc1ccc(F)cc1)C2]

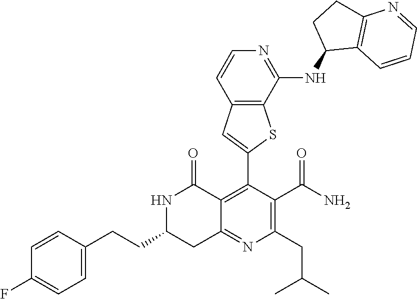 [SMILES: CC(C)Cc1nc2c(c(-c3cc4ccnc(N[C@H]5CCc6ncccc65)c4s3)c1C(N)=O)C(=O)N[C@@H](CCc1ccc(F)cc1)C2]

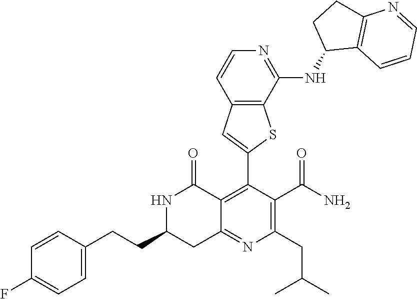 [SMILES: CC(C)Cc1nc2c(c(-c3cc4ccnc(N[C@@H]5CCc6ncccc65)c4s3)c1C(N)=O)C(=O)N[C@H](CCc1ccc(F)cc1)C2]

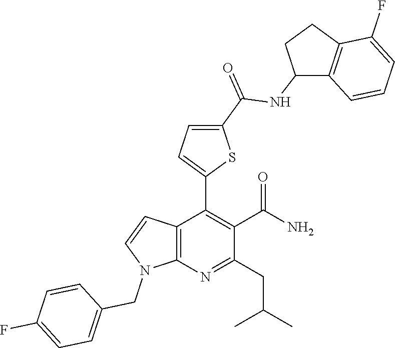 [SMILES: CC(C)Cc1nc2c(ccn2Cc2ccc(F)cc2)c(-c2ccc(C(=O)NC3CCc4c(F)cccc43)s2)c1C(N)=O]

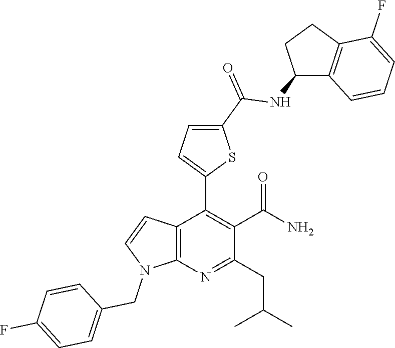 [SMILES: CC(C)Cc1nc2c(ccn2Cc2ccc(F)cc2)c(-c2ccc(C(=O)N[C@H]3CCc4c(F)cccc43)s2)c1C(N)=O]

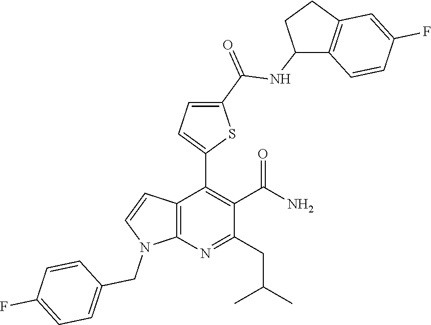 [SMILES: CC(C)Cc1nc2c(ccn2Cc2ccc(F)cc2)c(-c2ccc(C(=O)NC3CCc4cc(F)ccc43)s2)c1C(N)=O]

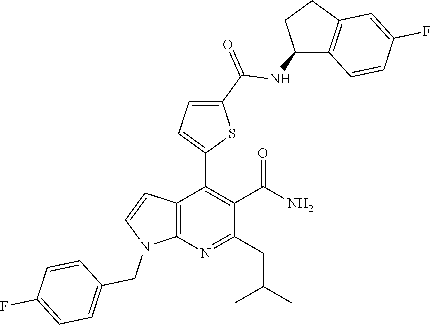 [SMILES: CC(C)Cc1nc2c(ccn2Cc2ccc(F)cc2)c(-c2ccc(C(=O)N[C@H]3CCc4cc(F)ccc43)s2)c1C(N)=O]

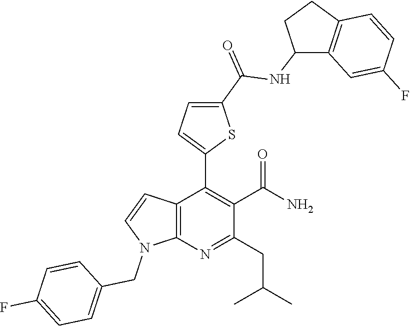 [SMILES: CC(C)Cc1nc2c(ccn2Cc2ccc(F)cc2)c(-c2ccc(C(=O)NC3CCc4ccc(F)cc43)s2)c1C(N)=O]

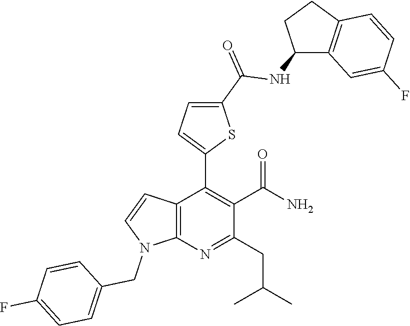 [SMILES: CC(C)Cc1nc2c(ccn2Cc2ccc(F)cc2)c(-c2ccc(C(=O)N[C@H]3CCc4ccc(F)cc43)s2)c1C(N)=O]

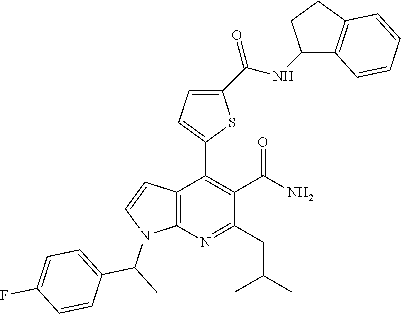 [SMILES: CC(C)Cc1nc2c(ccn2C(C)c2ccc(F)cc2)c(-c2ccc(C(=O)NC3CCc4ccccc43)s2)c1C(N)=O]

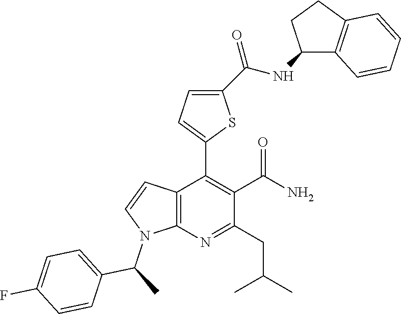 [SMILES: CC(C)Cc1nc2c(ccn2[C@@H](C)c2ccc(F)cc2)c(-c2ccc(C(=O)N[C@H]3CCc4ccccc43)s2)c1C(N)=O]

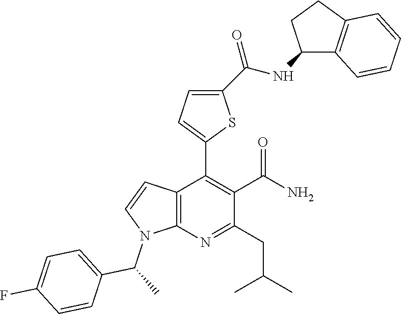 [SMILES: CC(C)Cc1nc2c(ccn2[C@H](C)c2ccc(F)cc2)c(-c2ccc(C(=O)N[C@H]3CCc4ccccc43)s2)c1C(N)=O]

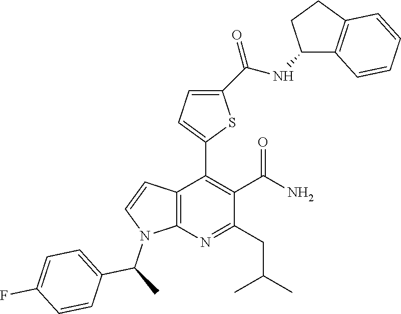 [SMILES: CC(C)Cc1nc2c(ccn2[C@@H](C)c2ccc(F)cc2)c(-c2ccc(C(=O)N[C@@H]3CCc4ccccc43)s2)c1C(N)=O]

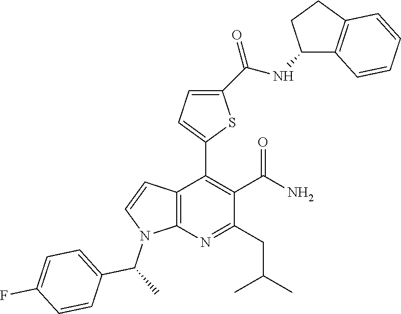 [SMILES: CC(C)Cc1nc2c(ccn2[C@H](C)c2ccc(F)cc2)c(-c2ccc(C(=O)N[C@@H]3CCc4ccccc43)s2)c1C(N)=O]

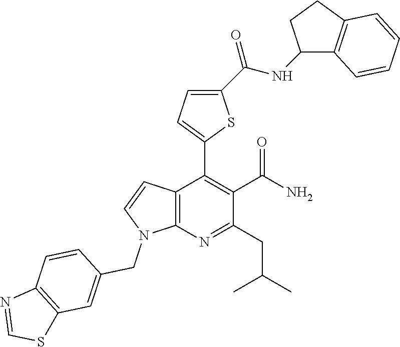 [SMILES: CC(C)Cc1nc2c(ccn2Cc2ccc3ncsc3c2)c(-c2ccc(C(=O)NC3CCc4ccccc43)s2)c1C(N)=O]

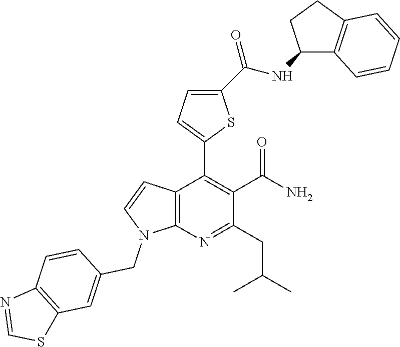 [SMILES: CC(C)Cc1nc2c(ccn2Cc2ccc3ncsc3c2)c(-c2ccc(C(=O)N[C@H]3CCc4ccccc43)s2)c1C(N)=O]

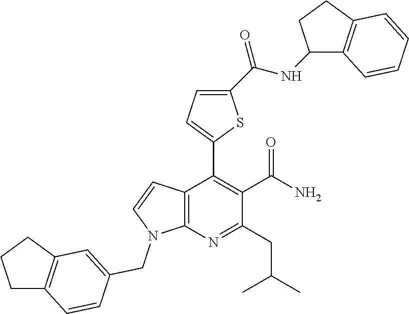 [SMILES: CC(C)Cc1nc2c(ccn2Cc2ccc3c(c2)CCC3)c(-c2ccc(C(=O)NC3CCc4ccccc43)s2)c1C(N)=O]

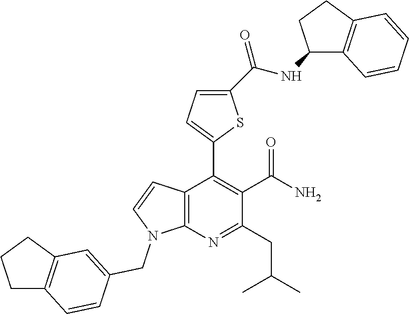 [SMILES: CC(C)Cc1nc2c(ccn2Cc2ccc3c(c2)CCC3)c(-c2ccc(C(=O)N[C@H]3CCc4ccccc43)s2)c1C(N)=O]

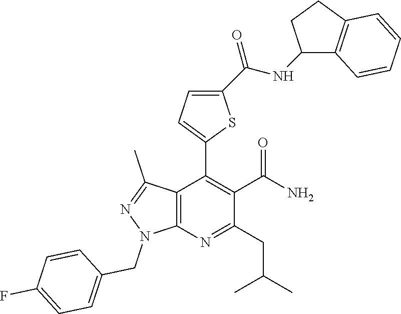 [SMILES: Cc1nn(Cc2ccc(F)cc2)c2nc(CC(C)C)c(C(N)=O)c(-c3ccc(C(=O)NC4CCc5ccccc54)s3)c12]

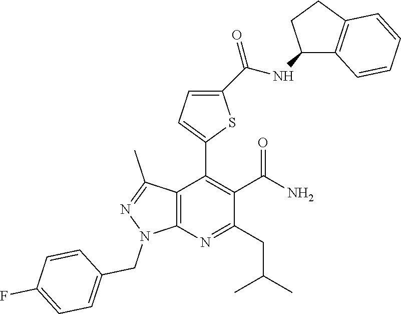 [SMILES: Cc1nn(Cc2ccc(F)cc2)c2nc(CC(C)C)c(C(N)=O)c(-c3ccc(C(=O)N[C@H]4CCc5ccccc54)s3)c12]

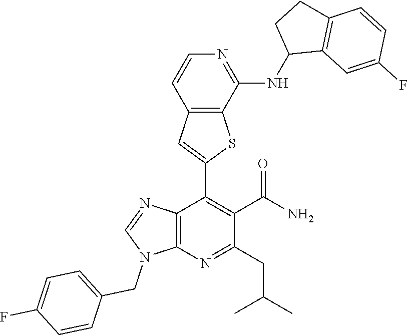 [SMILES: CC(C)Cc1nc2c(ncn2Cc2ccc(F)cc2)c(-c2cc3ccnc(NC4CCc5ccc(F)cc54)c3s2)c1C(N)=O]

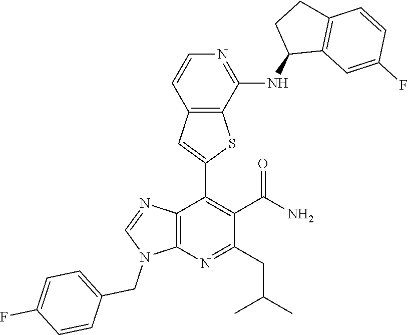 [SMILES: CC(C)Cc1nc2c(ncn2Cc2ccc(F)cc2)c(-c2cc3ccnc(N[C@H]4CCc5ccc(F)cc54)c3s2)c1C(N)=O]

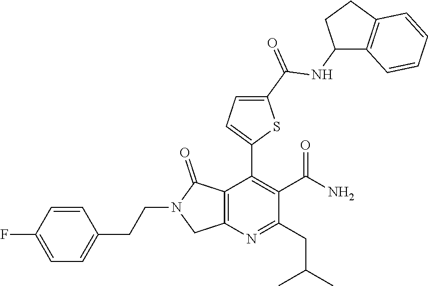 [SMILES: CC(C)Cc1nc2c(c(-c3ccc(C(=O)NC4CCc5ccccc54)s3)c1C(N)=O)C(=O)N(CCc1ccc(F)cc1)C2]

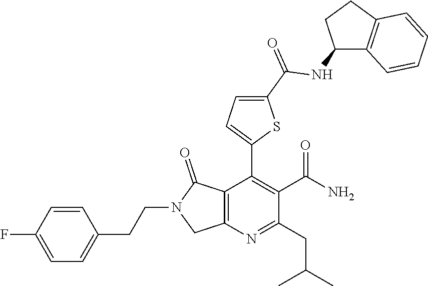 [SMILES: CC(C)Cc1nc2c(c(-c3ccc(C(=O)N[C@H]4CCc5ccccc54)s3)c1C(N)=O)C(=O)N(CCc1ccc(F)cc1)C2]

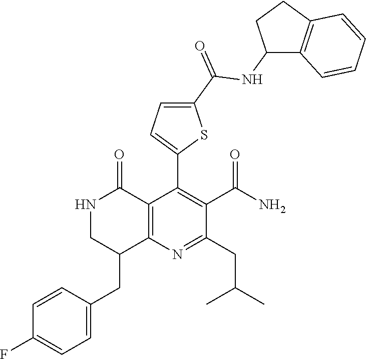 [SMILES: CC(C)Cc1nc2c(c(-c3ccc(C(=O)NC4CCc5ccccc54)s3)c1C(N)=O)C(=O)NCC2Cc1ccc(F)cc1]

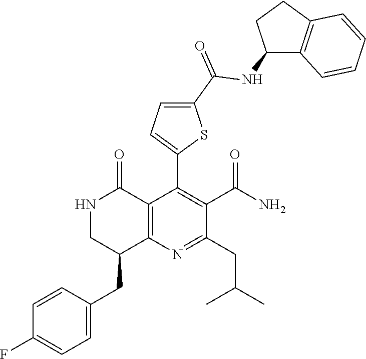 [SMILES: CC(C)Cc1nc2c(c(-c3ccc(C(=O)N[C@H]4CCc5ccccc54)s3)c1C(N)=O)C(=O)NC[C@@H]2Cc1ccc(F)cc1]

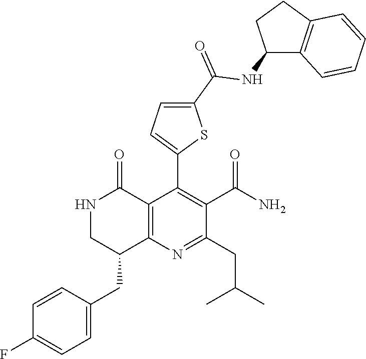 [SMILES: CC(C)Cc1nc2c(c(-c3ccc(C(=O)N[C@H]4CCc5ccccc54)s3)c1C(N)=O)C(=O)NC[C@H]2Cc1ccc(F)cc1]

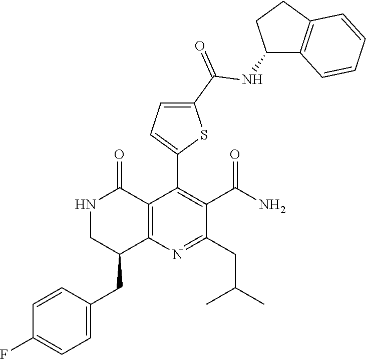 [SMILES: CC(C)Cc1nc2c(c(-c3ccc(C(=O)N[C@@H]4CCc5ccccc54)s3)c1C(N)=O)C(=O)NC[C@@H]2Cc1ccc(F)cc1]

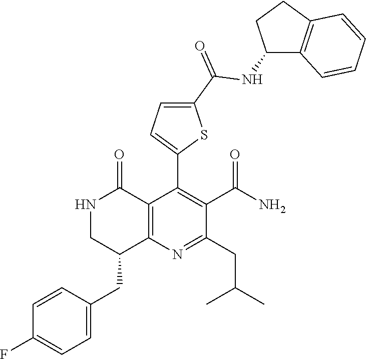 [SMILES: CC(C)Cc1nc2c(c(-c3ccc(C(=O)N[C@@H]4CCc5ccccc54)s3)c1C(N)=O)C(=O)NC[C@H]2Cc1ccc(F)cc1]

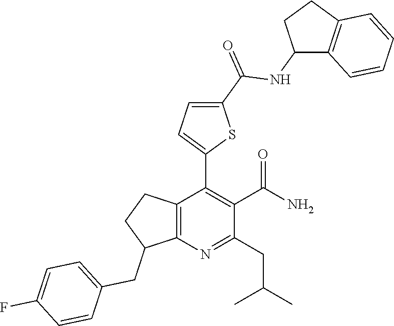 [SMILES: CC(C)Cc1nc2c(c(-c3ccc(C(=O)NC4CCc5ccccc54)s3)c1C(N)=O)CCC2Cc1ccc(F)cc1]

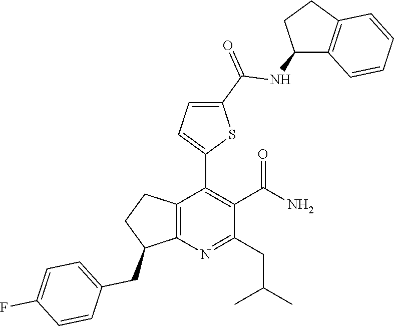 [SMILES: CC(C)Cc1nc2c(c(-c3ccc(C(=O)N[C@H]4CCc5ccccc54)s3)c1C(N)=O)CC[C@@H]2Cc1ccc(F)cc1]

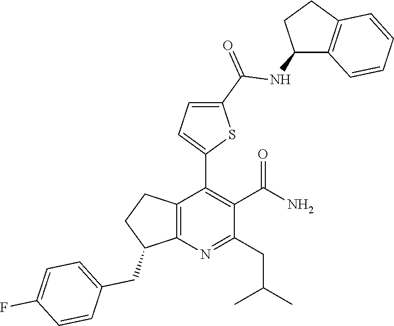 [SMILES: CC(C)Cc1nc2c(c(-c3ccc(C(=O)N[C@H]4CCc5ccccc54)s3)c1C(N)=O)CC[C@H]2Cc1ccc(F)cc1]

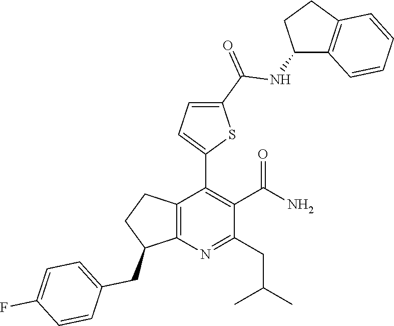 [SMILES: CC(C)Cc1nc2c(c(-c3ccc(C(=O)N[C@@H]4CCc5ccccc54)s3)c1C(N)=O)CC[C@@H]2Cc1ccc(F)cc1]

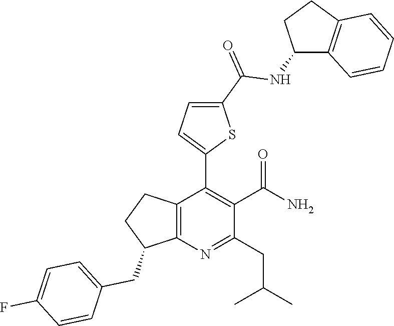 [SMILES: CC(C)Cc1nc2c(c(-c3ccc(C(=O)N[C@@H]4CCc5ccccc54)s3)c1C(N)=O)CC[C@H]2Cc1ccc(F)cc1]

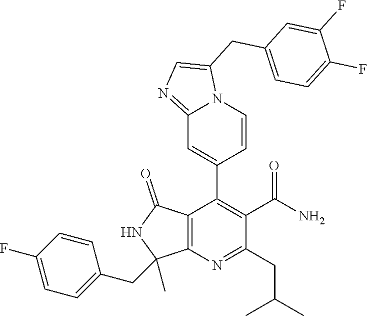 [SMILES: CC(C)Cc1nc2c(c(-c3ccn4c(Cc5ccc(F)c(F)c5)cnc4c3)c1C(N)=O)C(=O)NC2(C)Cc1ccc(F)cc1]

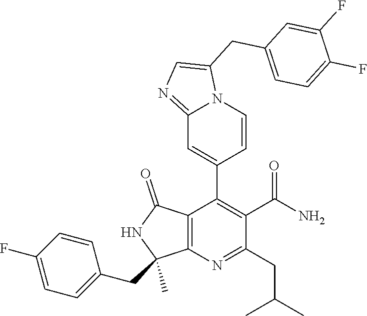 [SMILES: CC(C)Cc1nc2c(c(-c3ccn4c(Cc5ccc(F)c(F)c5)cnc4c3)c1C(N)=O)C(=O)N[C@]2(C)Cc1ccc(F)cc1]

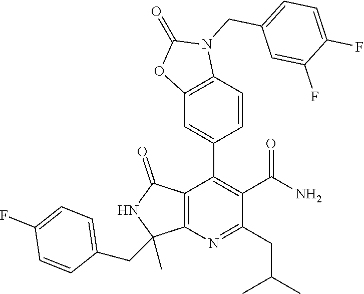 [SMILES: CC(C)Cc1nc2c(c(-c3ccc4c(c3)oc(=O)n4Cc3ccc(F)c(F)c3)c1C(N)=O)C(=O)NC2(C)Cc1ccc(F)cc1]

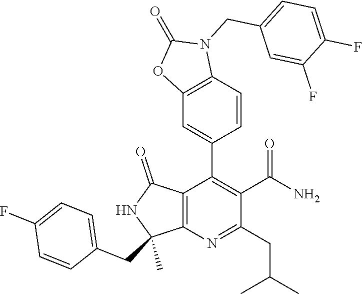 [SMILES: CC(C)Cc1nc2c(c(-c3ccc4c(c3)oc(=O)n4Cc3ccc(F)c(F)c3)c1C(N)=O)C(=O)N[C@]2(C)Cc1ccc(F)cc1]

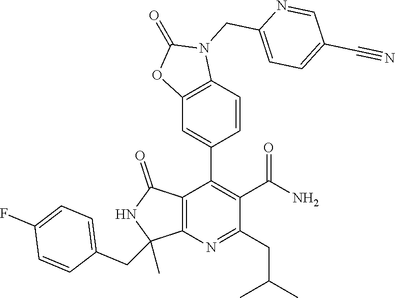 [SMILES: CC(C)Cc1nc2c(c(-c3ccc4c(c3)oc(=O)n4Cc3ccc(C#N)cn3)c1C(N)=O)C(=O)NC2(C)Cc1ccc(F)cc1]

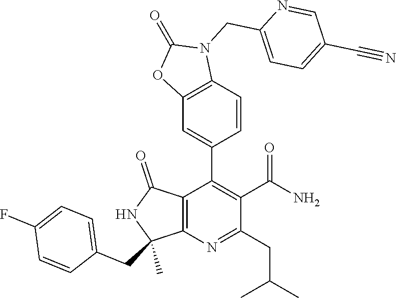 [SMILES: CC(C)Cc1nc2c(c(-c3ccc4c(c3)oc(=O)n4Cc3ccc(C#N)cn3)c1C(N)=O)C(=O)N[C@]2(C)Cc1ccc(F)cc1]

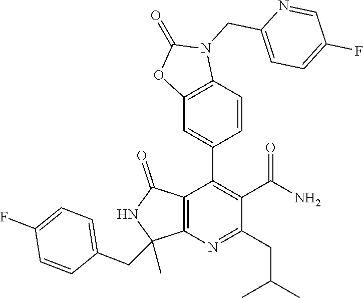 [SMILES: CC(C)Cc1nc2c(c(-c3ccc4c(c3)oc(=O)n4Cc3ccc(F)cn3)c1C(N)=O)C(=O)NC2(C)Cc1ccc(F)cc1]

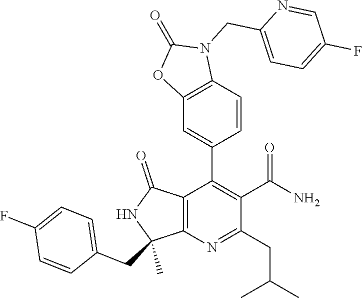 [SMILES: CC(C)Cc1nc2c(c(-c3ccc4c(c3)oc(=O)n4Cc3ccc(F)cn3)c1C(N)=O)C(=O)N[C@]2(C)Cc1ccc(F)cc1]

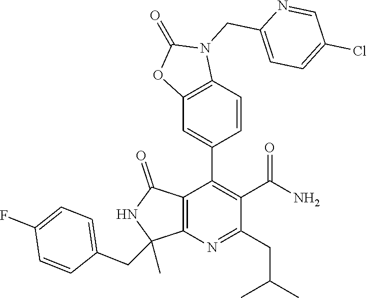 [SMILES: CC(C)Cc1nc2c(c(-c3ccc4c(c3)oc(=O)n4Cc3ccc(Cl)cn3)c1C(N)=O)C(=O)NC2(C)Cc1ccc(F)cc1]

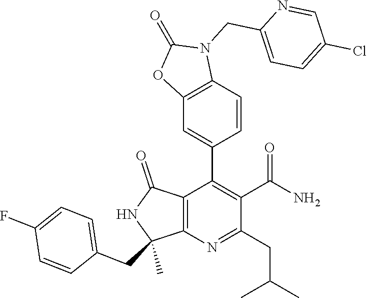 [SMILES: CC(C)Cc1nc2c(c(-c3ccc4c(c3)oc(=O)n4Cc3ccc(Cl)cn3)c1C(N)=O)C(=O)N[C@]2(C)Cc1ccc(F)cc1]

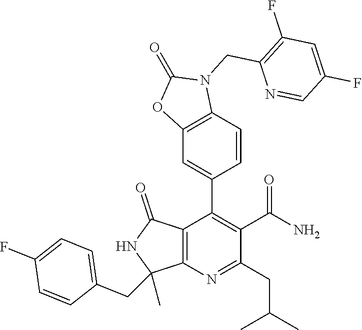 [SMILES: CC(C)Cc1nc2c(c(-c3ccc4c(c3)oc(=O)n4Cc3ncc(F)cc3F)c1C(N)=O)C(=O)NC2(C)Cc1ccc(F)cc1]

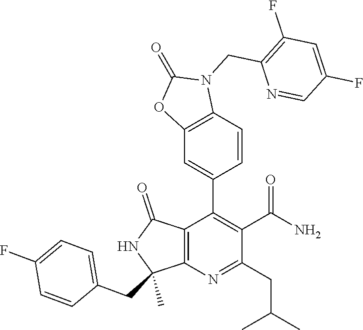 [SMILES: CC(C)Cc1nc2c(c(-c3ccc4c(c3)oc(=O)n4Cc3ncc(F)cc3F)c1C(N)=O)C(=O)N[C@]2(C)Cc1ccc(F)cc1]

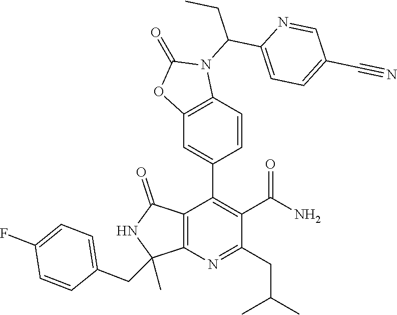 [SMILES: CCC(c1ccc(C#N)cn1)n1c(=O)oc2cc(-c3c(C(N)=O)c(CC(C)C)nc4c3C(=O)NC4(C)Cc3ccc(F)cc3)ccc21]

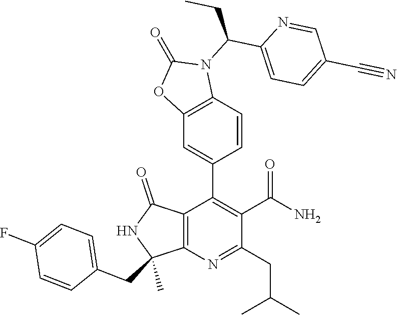 [SMILES: CC[C@@H](c1ccc(C#N)cn1)n1c(=O)oc2cc(-c3c(C(N)=O)c(CC(C)C)nc4c3C(=O)N[C@]4(C)Cc3ccc(F)cc3)ccc21]

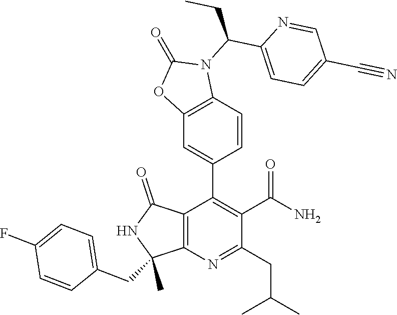 [SMILES: CC[C@@H](c1ccc(C#N)cn1)n1c(=O)oc2cc(-c3c(C(N)=O)c(CC(C)C)nc4c3C(=O)N[C@@]4(C)Cc3ccc(F)cc3)ccc21]

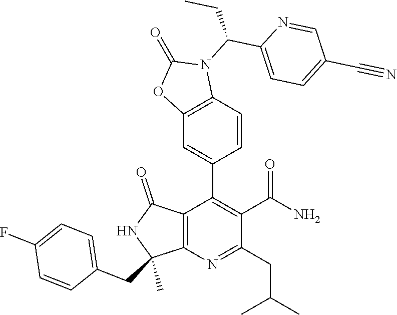 [SMILES: CC[C@H](c1ccc(C#N)cn1)n1c(=O)oc2cc(-c3c(C(N)=O)c(CC(C)C)nc4c3C(=O)N[C@]4(C)Cc3ccc(F)cc3)ccc21]

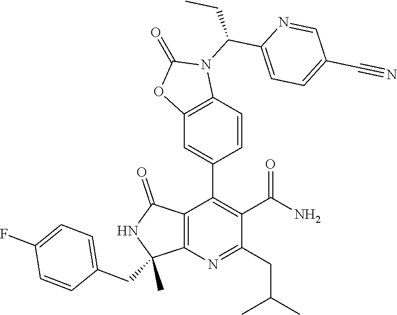 [SMILES: CC[C@H](c1ccc(C#N)cn1)n1c(=O)oc2cc(-c3c(C(N)=O)c(CC(C)C)nc4c3C(=O)N[C@@]4(C)Cc3ccc(F)cc3)ccc21]

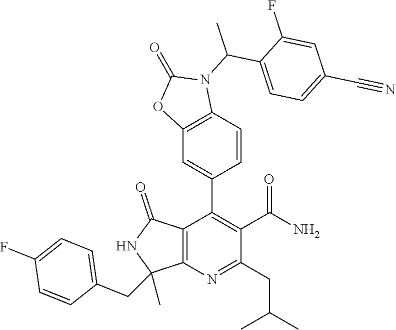 [SMILES: CC(C)Cc1nc2c(c(-c3ccc4c(c3)oc(=O)n4C(C)c3ccc(C#N)cc3F)c1C(N)=O)C(=O)NC2(C)Cc1ccc(F)cc1]

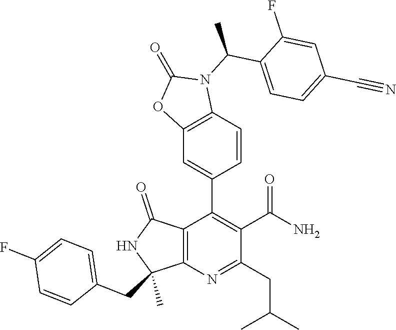 [SMILES: CC(C)Cc1nc2c(c(-c3ccc4c(c3)oc(=O)n4[C@@H](C)c3ccc(C#N)cc3F)c1C(N)=O)C(=O)N[C@]2(C)Cc1ccc(F)cc1]

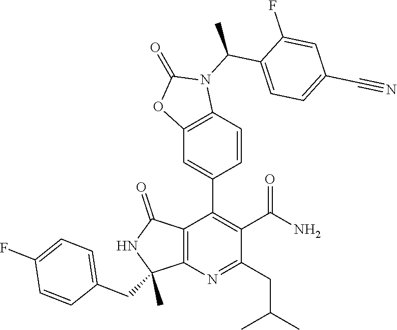 [SMILES: CC(C)Cc1nc2c(c(-c3ccc4c(c3)oc(=O)n4[C@@H](C)c3ccc(C#N)cc3F)c1C(N)=O)C(=O)N[C@@]2(C)Cc1ccc(F)cc1]

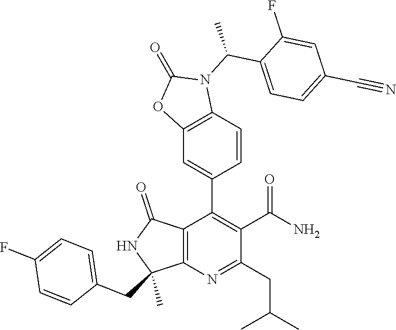 [SMILES: CC(C)Cc1nc2c(c(-c3ccc4c(c3)oc(=O)n4[C@H](C)c3ccc(C#N)cc3F)c1C(N)=O)C(=O)N[C@]2(C)Cc1ccc(F)cc1]

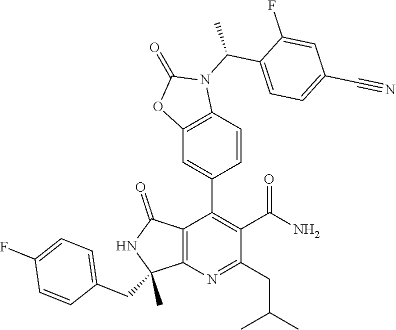 [SMILES: CC(C)Cc1nc2c(c(-c3ccc4c(c3)oc(=O)n4[C@H](C)c3ccc(C#N)cc3F)c1C(N)=O)C(=O)N[C@@]2(C)Cc1ccc(F)cc1]

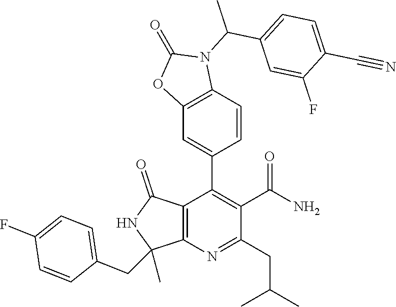 [SMILES: CC(C)Cc1nc2c(c(-c3ccc4c(c3)oc(=O)n4C(C)c3ccc(C#N)c(F)c3)c1C(N)=O)C(=O)NC2(C)Cc1ccc(F)cc1]

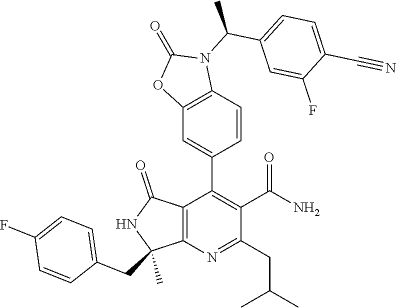 [SMILES: CC(C)Cc1nc2c(c(-c3ccc4c(c3)oc(=O)n4[C@@H](C)c3ccc(C#N)c(F)c3)c1C(N)=O)C(=O)N[C@]2(C)Cc1ccc(F)cc1]

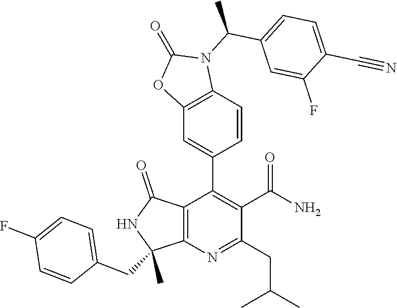 [SMILES: CC(C)Cc1nc2c(c(-c3ccc4c(c3)oc(=O)n4[C@@H](C)c3ccc(C#N)c(F)c3)c1C(N)=O)C(=O)N[C@@]2(C)Cc1ccc(F)cc1]

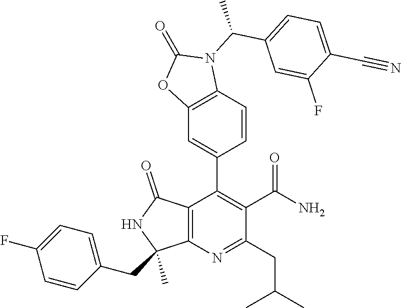 [SMILES: CC(C)Cc1nc2c(c(-c3ccc4c(c3)oc(=O)n4[C@H](C)c3ccc(C#N)c(F)c3)c1C(N)=O)C(=O)N[C@]2(C)Cc1ccc(F)cc1]

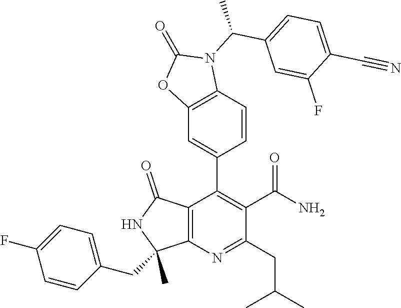 [SMILES: CC(C)Cc1nc2c(c(-c3ccc4c(c3)oc(=O)n4[C@H](C)c3ccc(C#N)c(F)c3)c1C(N)=O)C(=O)N[C@@]2(C)Cc1ccc(F)cc1]

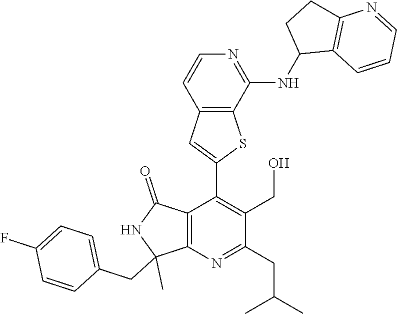 [SMILES: CC(C)Cc1nc2c(c(-c3cc4ccnc(NC5CCc6ncccc65)c4s3)c1CO)C(=O)NC2(C)Cc1ccc(F)cc1]

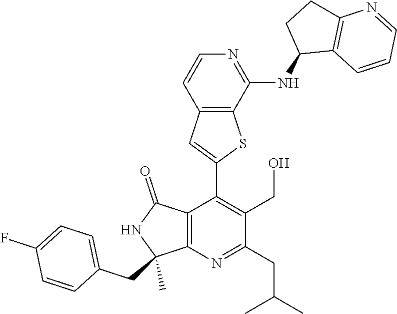 [SMILES: CC(C)Cc1nc2c(c(-c3cc4ccnc(N[C@H]5CCc6ncccc65)c4s3)c1CO)C(=O)N[C@]2(C)Cc1ccc(F)cc1]

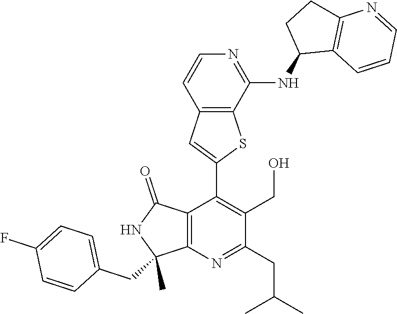 [SMILES: CC(C)Cc1nc2c(c(-c3cc4ccnc(N[C@H]5CCc6ncccc65)c4s3)c1CO)C(=O)N[C@@]2(C)Cc1ccc(F)cc1]

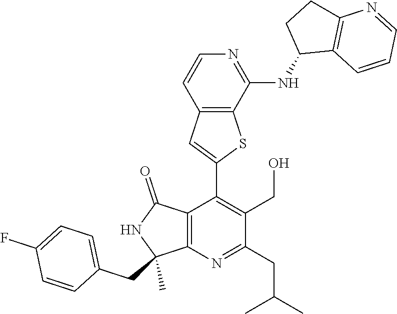 [SMILES: CC(C)Cc1nc2c(c(-c3cc4ccnc(N[C@@H]5CCc6ncccc65)c4s3)c1CO)C(=O)N[C@]2(C)Cc1ccc(F)cc1]

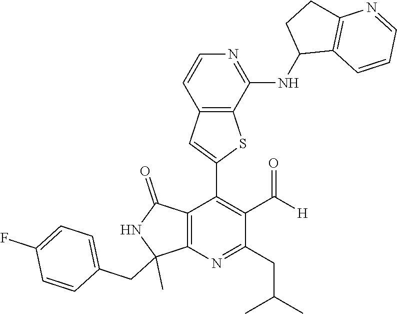 [SMILES: CC(C)Cc1nc2c(c(-c3cc4ccnc(NC5CCc6ncccc65)c4s3)c1C=O)C(=O)NC2(C)Cc1ccc(F)cc1]

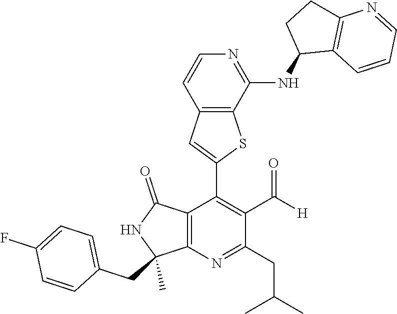 [SMILES: CC(C)Cc1nc2c(c(-c3cc4ccnc(N[C@H]5CCc6ncccc65)c4s3)c1C=O)C(=O)N[C@]2(C)Cc1ccc(F)cc1]

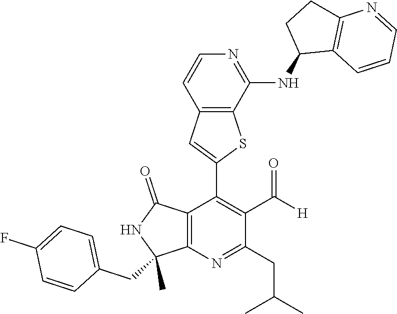 [SMILES: CC(C)Cc1nc2c(c(-c3cc4ccnc(N[C@H]5CCc6ncccc65)c4s3)c1C=O)C(=O)N[C@@]2(C)Cc1ccc(F)cc1]

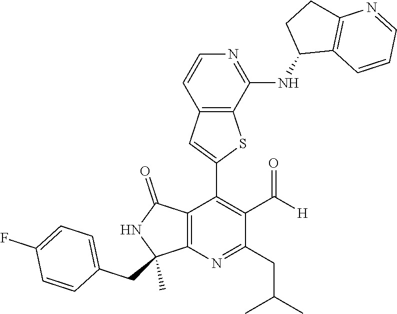 [SMILES: CC(C)Cc1nc2c(c(-c3cc4ccnc(N[C@@H]5CCc6ncccc65)c4s3)c1C=O)C(=O)N[C@]2(C)Cc1ccc(F)cc1]

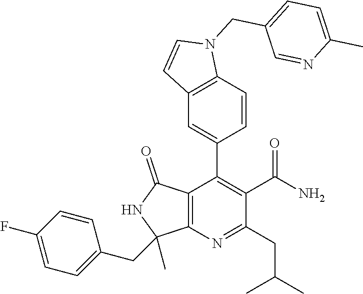 [SMILES: Cc1ccc(Cn2ccc3cc(-c4c(C(N)=O)c(CC(C)C)nc5c4C(=O)NC5(C)Cc4ccc(F)cc4)ccc32)cn1]

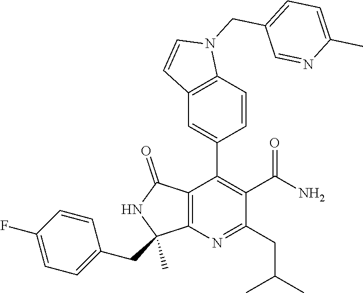 [SMILES: Cc1ccc(Cn2ccc3cc(-c4c(C(N)=O)c(CC(C)C)nc5c4C(=O)N[C@]5(C)Cc4ccc(F)cc4)ccc32)cn1]

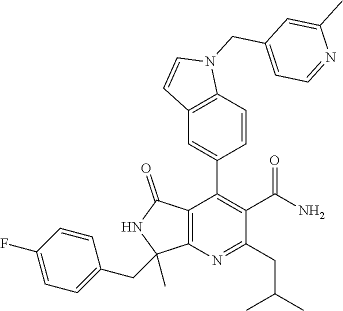 [SMILES: Cc1cc(Cn2ccc3cc(-c4c(C(N)=O)c(CC(C)C)nc5c4C(=O)NC5(C)Cc4ccc(F)cc4)ccc32)ccn1]

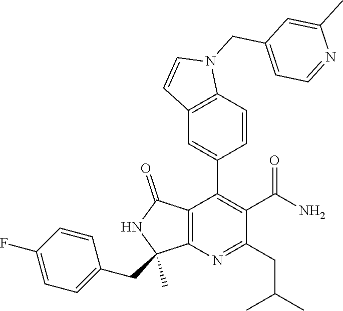 [SMILES: Cc1cc(Cn2ccc3cc(-c4c(C(N)=O)c(CC(C)C)nc5c4C(=O)N[C@]5(C)Cc4ccc(F)cc4)ccc32)ccn1]

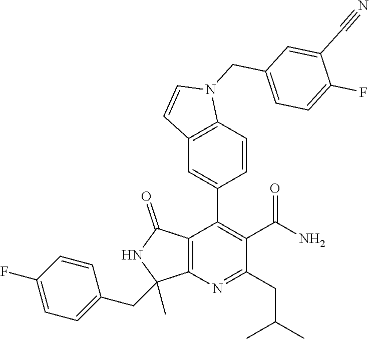 [SMILES: CC(C)Cc1nc2c(c(-c3ccc4c(ccn4Cc4ccc(F)c(C#N)c4)c3)c1C(N)=O)C(=O)NC2(C)Cc1ccc(F)cc1]

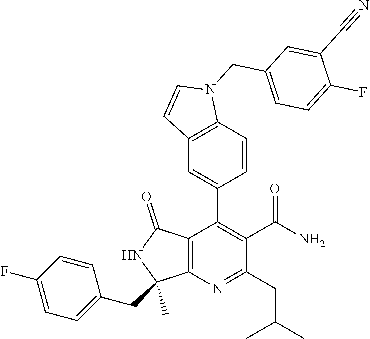 [SMILES: CC(C)Cc1nc2c(c(-c3ccc4c(ccn4Cc4ccc(F)c(C#N)c4)c3)c1C(N)=O)C(=O)N[C@]2(C)Cc1ccc(F)cc1]

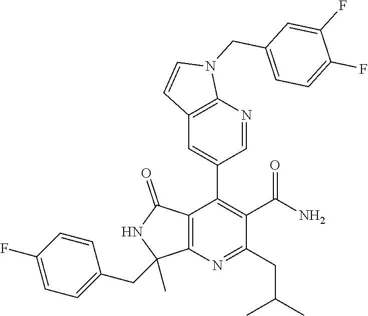 [SMILES: CC(C)Cc1nc2c(c(-c3cnc4c(ccn4Cc4ccc(F)c(F)c4)c3)c1C(N)=O)C(=O)NC2(C)Cc1ccc(F)cc1]

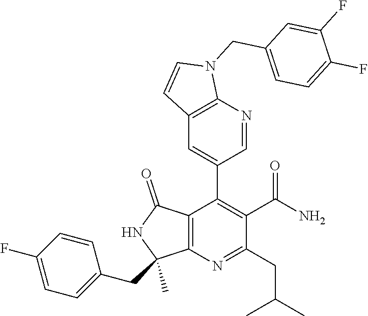 [SMILES: CC(C)Cc1nc2c(c(-c3cnc4c(ccn4Cc4ccc(F)c(F)c4)c3)c1C(N)=O)C(=O)N[C@]2(C)Cc1ccc(F)cc1]

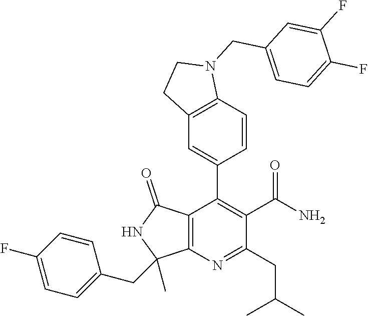 [SMILES: CC(C)Cc1nc2c(c(-c3ccc4c(c3)CCN4Cc3ccc(F)c(F)c3)c1C(N)=O)C(=O)NC2(C)Cc1ccc(F)cc1]

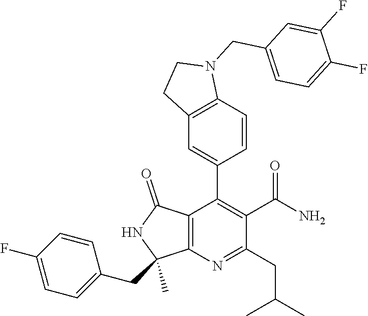 [SMILES: CC(C)Cc1nc2c(c(-c3ccc4c(c3)CCN4Cc3ccc(F)c(F)c3)c1C(N)=O)C(=O)N[C@]2(C)Cc1ccc(F)cc1]

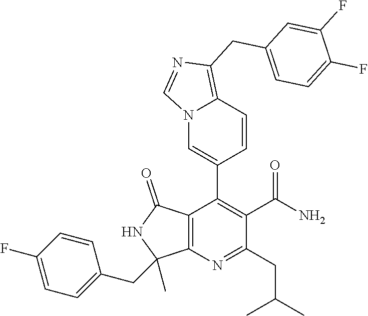 [SMILES: CC(C)Cc1nc2c(c(-c3ccc4c(Cc5ccc(F)c(F)c5)ncn4c3)c1C(N)=O)C(=O)NC2(C)Cc1ccc(F)cc1]

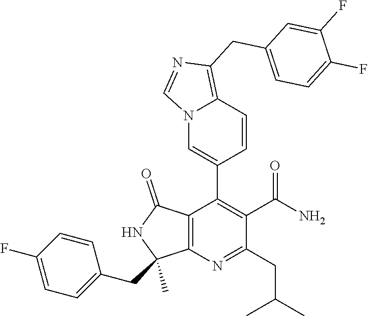 [SMILES: CC(C)Cc1nc2c(c(-c3ccc4c(Cc5ccc(F)c(F)c5)ncn4c3)c1C(N)=O)C(=O)N[C@]2(C)Cc1ccc(F)cc1]

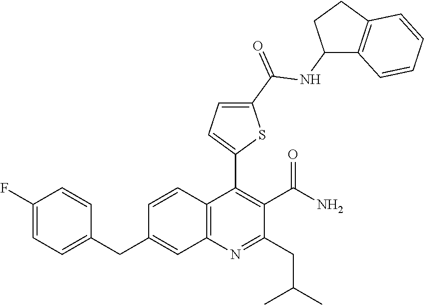 [SMILES: CC(C)Cc1nc2cc(Cc3ccc(F)cc3)ccc2c(-c2ccc(C(=O)NC3CCc4ccccc43)s2)c1C(N)=O]

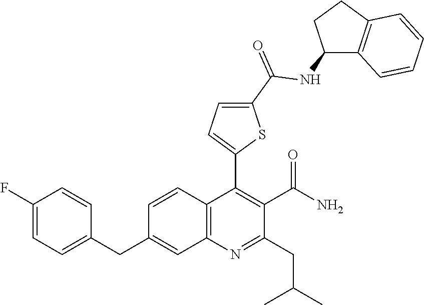 [SMILES: CC(C)Cc1nc2cc(Cc3ccc(F)cc3)ccc2c(-c2ccc(C(=O)N[C@H]3CCc4ccccc43)s2)c1C(N)=O]

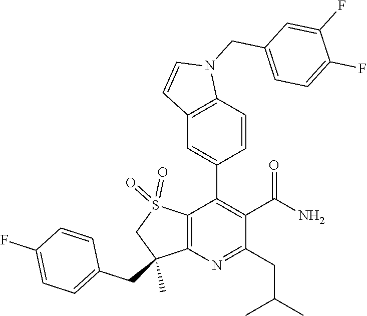 [SMILES: CC(C)Cc1nc2c(c(-c3ccc4c(ccn4Cc4ccc(F)c(F)c4)c3)c1C(N)=O)S(=O)(=O)C[C@]2(C)Cc1ccc(F)cc1]

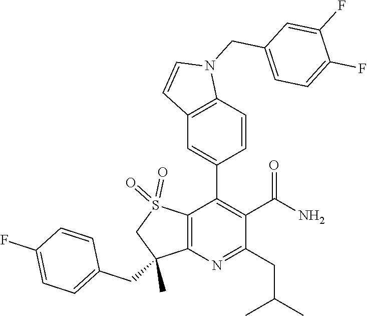 [SMILES: CC(C)Cc1nc2c(c(-c3ccc4c(ccn4Cc4ccc(F)c(F)c4)c3)c1C(N)=O)S(=O)(=O)C[C@@]2(C)Cc1ccc(F)cc1]

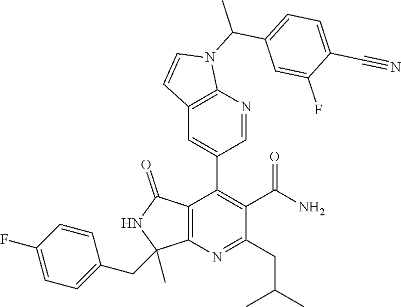 [SMILES: CC(C)Cc1nc2c(c(-c3cnc4c(ccn4C(C)c4ccc(C#N)c(F)c4)c3)c1C(N)=O)C(=O)NC2(C)Cc1ccc(F)cc1]

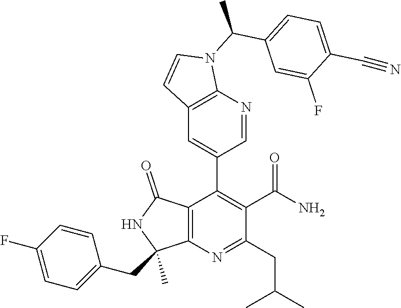 [SMILES: CC(C)Cc1nc2c(c(-c3cnc4c(ccn4[C@@H](C)c4ccc(C#N)c(F)c4)c3)c1C(N)=O)C(=O)N[C@]2(C)Cc1ccc(F)cc1]

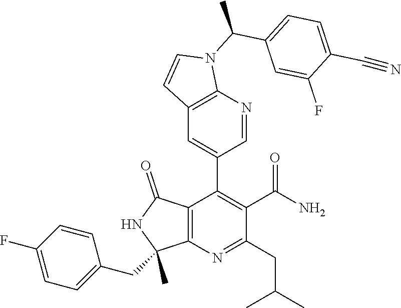 [SMILES: CC(C)Cc1nc2c(c(-c3cnc4c(ccn4[C@@H](C)c4ccc(C#N)c(F)c4)c3)c1C(N)=O)C(=O)N[C@@]2(C)Cc1ccc(F)cc1]

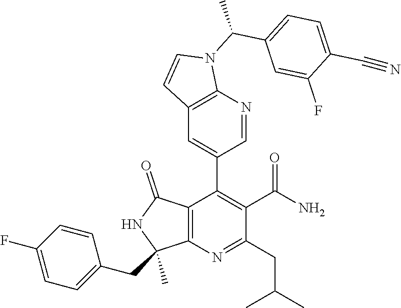 [SMILES: CC(C)Cc1nc2c(c(-c3cnc4c(ccn4[C@H](C)c4ccc(C#N)c(F)c4)c3)c1C(N)=O)C(=O)N[C@]2(C)Cc1ccc(F)cc1]

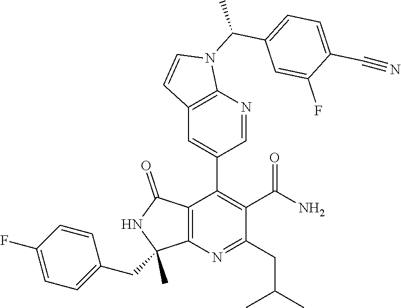 [SMILES: CC(C)Cc1nc2c(c(-c3cnc4c(ccn4[C@H](C)c4ccc(C#N)c(F)c4)c3)c1C(N)=O)C(=O)N[C@@]2(C)Cc1ccc(F)cc1]

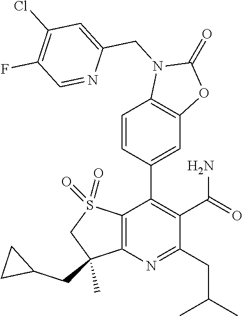 [SMILES: CC(C)Cc1nc2c(c(-c3ccc4c(c3)oc(=O)n4Cc3cc(Cl)c(F)cn3)c1C(N)=O)S(=O)(=O)C[C@]2(C)CC1CC1]

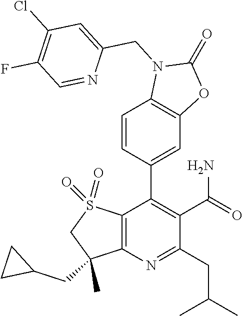 [SMILES: CC(C)Cc1nc2c(c(-c3ccc4c(c3)oc(=O)n4Cc3cc(Cl)c(F)cn3)c1C(N)=O)S(=O)(=O)C[C@@]2(C)CC1CC1]

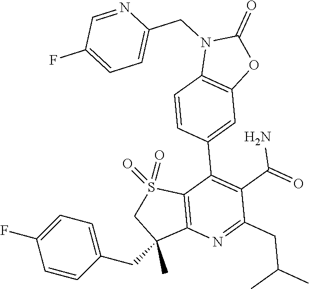 [SMILES: CC(C)Cc1nc2c(c(-c3ccc4c(c3)oc(=O)n4Cc3ccc(F)cn3)c1C(N)=O)S(=O)(=O)C[C@@]2(C)Cc1ccc(F)cc1]

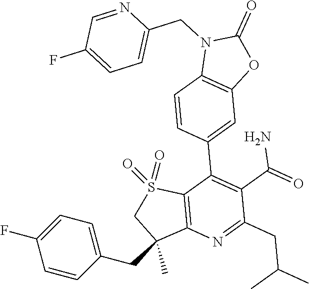 [SMILES: CC(C)Cc1nc2c(c(-c3ccc4c(c3)oc(=O)n4Cc3ccc(F)cn3)c1C(N)=O)S(=O)(=O)C[C@]2(C)Cc1ccc(F)cc1]

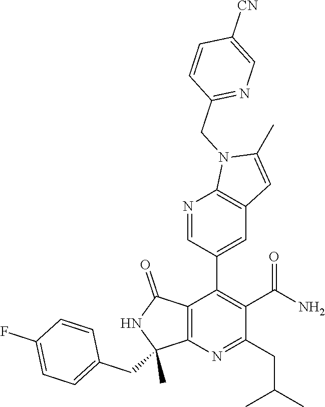 [SMILES: Cc1cc2cc(-c3c(C(N)=O)c(CC(C)C)nc4c3C(=O)N[C@@]4(C)Cc3ccc(F)cc3)cnc2n1Cc1ccc(C#N)cn1]

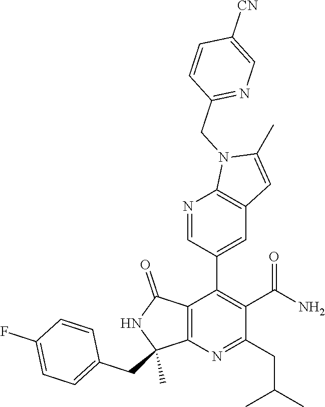 [SMILES: Cc1cc2cc(-c3c(C(N)=O)c(CC(C)C)nc4c3C(=O)N[C@]4(C)Cc3ccc(F)cc3)cnc2n1Cc1ccc(C#N)cn1]

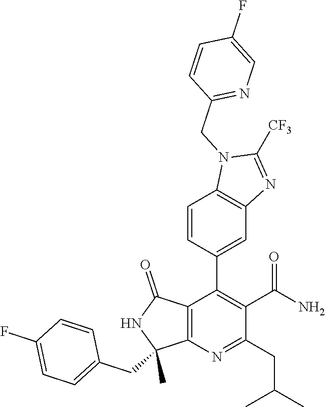 [SMILES: CC(C)Cc1nc2c(c(-c3ccc4c(c3)nc(C(F)(F)F)n4Cc3ccc(F)cn3)c1C(N)=O)C(=O)N[C@@]2(C)Cc1ccc(F)cc1]

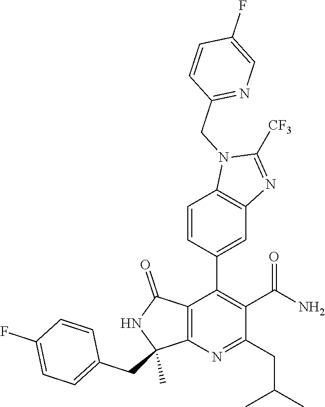 [SMILES: CC(C)Cc1nc2c(c(-c3ccc4c(c3)nc(C(F)(F)F)n4Cc3ccc(F)cn3)c1C(N)=O)C(=O)N[C@]2(C)Cc1ccc(F)cc1]

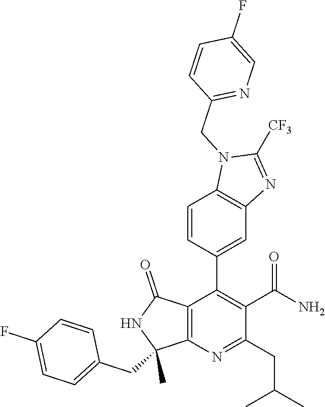 [SMILES: CC(C)Cc1nc2c(c(-c3ccc4c(c3)nc(C(F)(F)F)n4Cc3ccc(F)cn3)c1C(N)=O)C(=O)N[C@@]2(C)Cc1ccc(F)cc1]

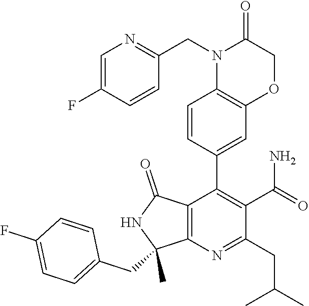 [SMILES: CC(C)Cc1nc2c(c(-c3ccc4c(c3)OCC(=O)N4Cc3ccc(F)cn3)c1C(N)=O)C(=O)N[C@@]2(C)Cc1ccc(F)cc1]

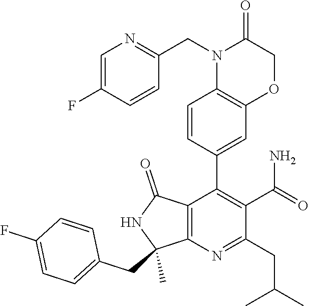 [SMILES: CC(C)Cc1nc2c(c(-c3ccc4c(c3)OCC(=O)N4Cc3ccc(F)cn3)c1C(N)=O)C(=O)N[C@]2(C)Cc1ccc(F)cc1]

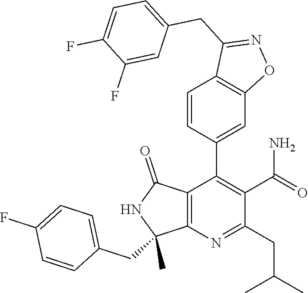 [SMILES: CC(C)Cc1nc2c(c(-c3ccc4c(Cc5ccc(F)c(F)c5)noc4c3)c1C(N)=O)C(=O)N[C@@]2(C)Cc1ccc(F)cc1]

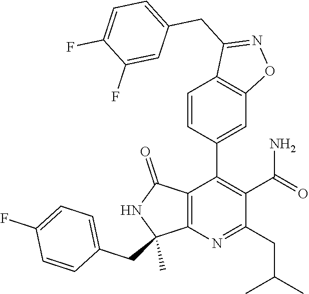 [SMILES: CC(C)Cc1nc2c(c(-c3ccc4c(Cc5ccc(F)c(F)c5)noc4c3)c1C(N)=O)C(=O)N[C@]2(C)Cc1ccc(F)cc1]

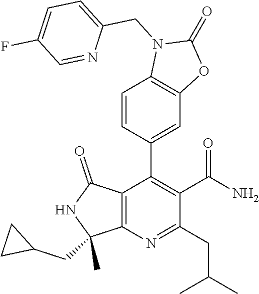 [SMILES: CC(C)Cc1nc2c(c(-c3ccc4c(c3)oc(=O)n4Cc3ccc(F)cn3)c1C(N)=O)C(=O)N[C@@]2(C)CC1CC1]

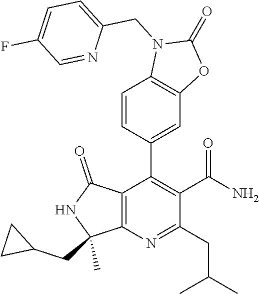 [SMILES: CC(C)Cc1nc2c(c(-c3ccc4c(c3)oc(=O)n4Cc3ccc(F)cn3)c1C(N)=O)C(=O)N[C@]2(C)CC1CC1]

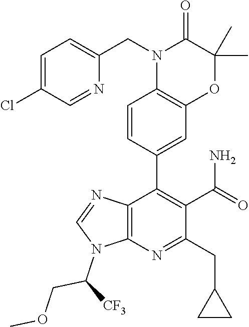

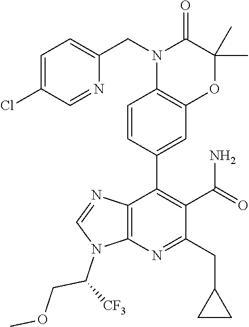

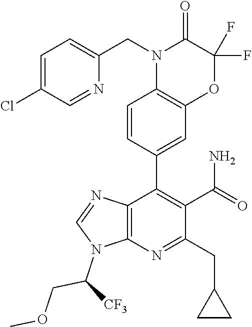 [SMILES: COC[C@@H](n1cnc2c(-c3ccc4c(c3)OC(F)(F)C(=O)N4Cc3ccc(Cl)cn3)c(C(N)=O)c(CC3CC3)nc21)C(F)(F)F]

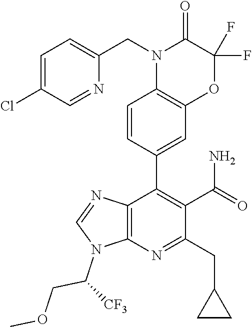 [SMILES: COC[C@H](n1cnc2c(-c3ccc4c(c3)OC(F)(F)C(=O)N4Cc3ccc(Cl)cn3)c(C(N)=O)c(CC3CC3)nc21)C(F)(F)F]

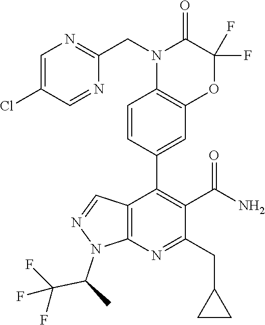 [SMILES: C[C@H](n1ncc2c(-c3ccc4c(c3)OC(F)(F)C(=O)N4Cc3ncc(Cl)cn3)c(C(N)=O)c(CC3CC3)nc21)C(F)(F)F]

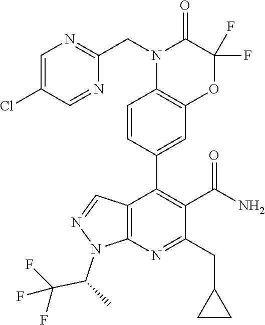 [SMILES: C[C@@H](n1ncc2c(-c3ccc4c(c3)OC(F)(F)C(=O)N4Cc3ncc(Cl)cn3)c(C(N)=O)c(CC3CC3)nc21)C(F)(F)F]

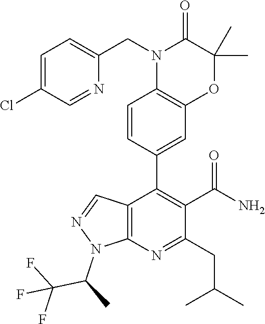

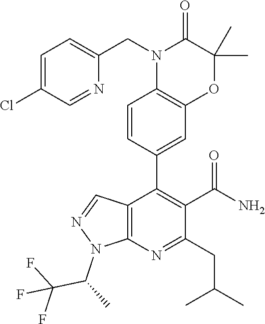

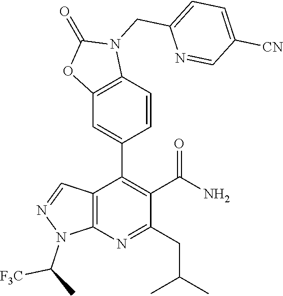 [SMILES: CC(C)Cc1nc2c(cnn2[C@@H](C)C(F)(F)F)c(-c2ccc3c(c2)oc(=O)n3Cc2ccc(C#N)cn2)c1C(N)=O]

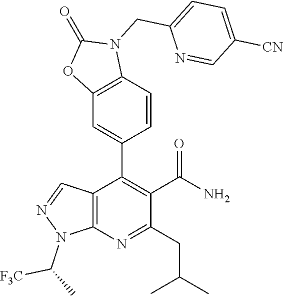 [SMILES: CC(C)Cc1nc2c(cnn2[C@H](C)C(F)(F)F)c(-c2ccc3c(c2)oc(=O)n3Cc2ccc(C#N)cn2)c1C(N)=O]

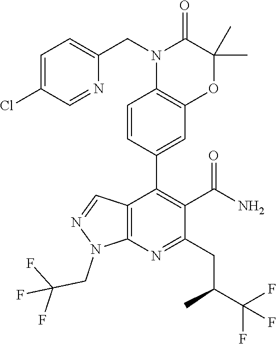 [SMILES: C[C@@H](Cc1nc2c(cnn2CC(F)(F)F)c(-c2ccc3c(c2)OC(C)(C)C(=O)N3Cc2ccc(Cl)cn2)c1C(N)=O)C(F)(F)F]

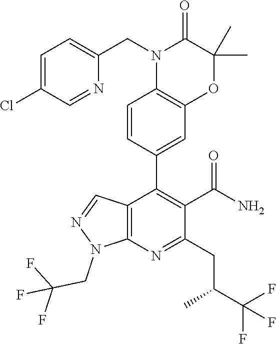 [SMILES: C[C@H](Cc1nc2c(cnn2CC(F)(F)F)c(-c2ccc3c(c2)OC(C)(C)C(=O)N3Cc2ccc(Cl)cn2)c1C(N)=O)C(F)(F)F]

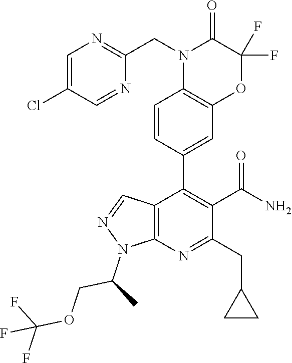 [SMILES: C[C@@H](COC(F)(F)F)n1ncc2c(-c3ccc4c(c3)OC(F)(F)C(=O)N4Cc3ncc(Cl)cn3)c(C(N)=O)c(CC3CC3)nc21]

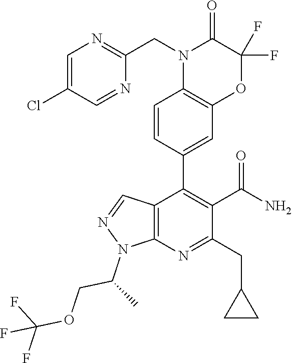 [SMILES: C[C@H](COC(F)(F)F)n1ncc2c(-c3ccc4c(c3)OC(F)(F)C(=O)N4Cc3ncc(Cl)cn3)c(C(N)=O)c(CC3CC3)nc21]

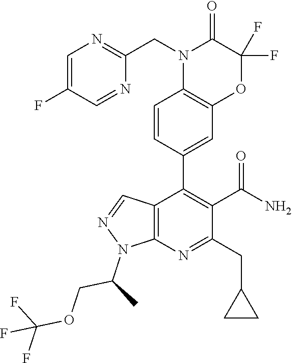 [SMILES: C[C@@H](COC(F)(F)F)n1ncc2c(-c3ccc4c(c3)OC(F)(F)C(=O)N4Cc3ncc(F)cn3)c(C(N)=O)c(CC3CC3)nc21]

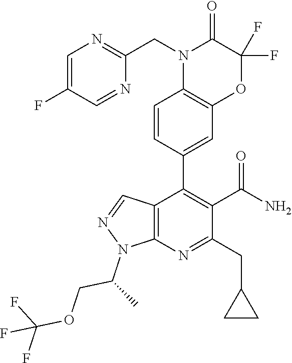 [SMILES: C[C@H](COC(F)(F)F)n1ncc2c(-c3ccc4c(c3)OC(F)(F)C(=O)N4Cc3ncc(F)cn3)c(C(N)=O)c(CC3CC3)nc21]

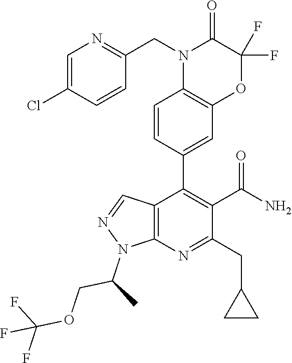 [SMILES: C[C@@H](COC(F)(F)F)n1ncc2c(-c3ccc4c(c3)OC(F)(F)C(=O)N4Cc3ccc(Cl)cn3)c(C(N)=O)c(CC3CC3)nc21]

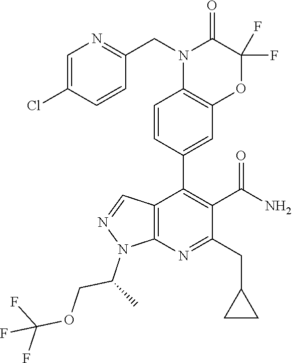 [SMILES: C[C@H](COC(F)(F)F)n1ncc2c(-c3ccc4c(c3)OC(F)(F)C(=O)N4Cc3ccc(Cl)cn3)c(C(N)=O)c(CC3CC3)nc21]

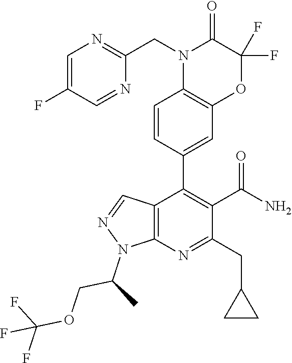 [SMILES: C[C@@H](COC(F)(F)F)n1ncc2c(-c3ccc4c(c3)OC(F)(F)C(=O)N4Cc3ncc(F)cn3)c(C(N)=O)c(CC3CC3)nc21]

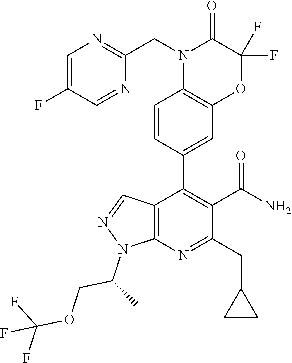 [SMILES: C[C@H](COC(F)(F)F)n1ncc2c(-c3ccc4c(c3)OC(F)(F)C(=O)N4Cc3ncc(F)cn3)c(C(N)=O)c(CC3CC3)nc21]

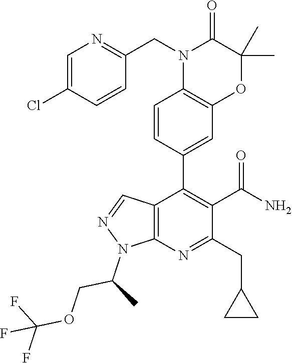 [SMILES: C[C@@H](COC(F)(F)F)n1ncc2c(-c3ccc4c(c3)OC(C)(C)C(=O)N4Cc3ccc(Cl)cn3)c(C(N)=O)c(CC3CC3)nc21]

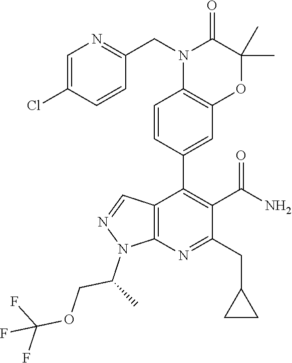 [SMILES: C[C@H](COC(F)(F)F)n1ncc2c(-c3ccc4c(c3)OC(C)(C)C(=O)N4Cc3ccc(Cl)cn3)c(C(N)=O)c(CC3CC3)nc21]

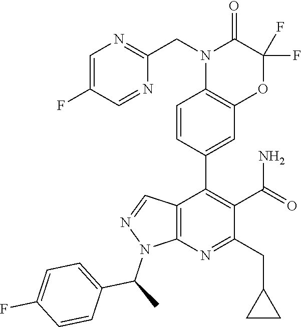 [SMILES: C[C@@H](c1ccc(F)cc1)n1ncc2c(-c3ccc4c(c3)OC(F)(F)C(=O)N4Cc3ncc(F)cn3)c(C(N)=O)c(CC3CC3)nc21]

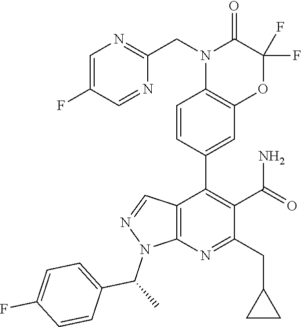 [SMILES: C[C@H](c1ccc(F)cc1)n1ncc2c(-c3ccc4c(c3)OC(F)(F)C(=O)N4Cc3ncc(F)cn3)c(C(N)=O)c(CC3CC3)nc21]

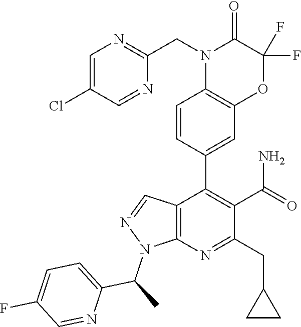 [SMILES: C[C@@H](c1ccc(F)cn1)n1ncc2c(-c3ccc4c(c3)OC(F)(F)C(=O)N4Cc3ncc(Cl)cn3)c(C(N)=O)c(CC3CC3)nc21]

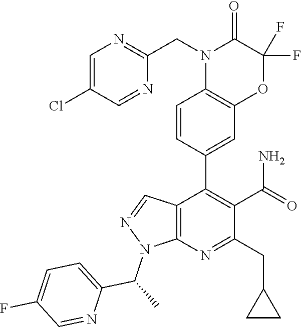 [SMILES: C[C@H](c1ccc(F)cn1)n1ncc2c(-c3ccc4c(c3)OC(F)(F)C(=O)N4Cc3ncc(Cl)cn3)c(C(N)=O)c(CC3CC3)nc21]

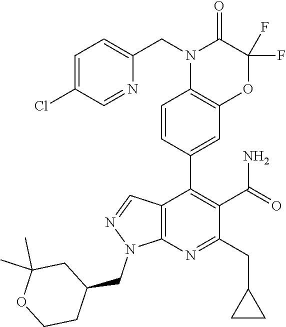 [SMILES: CC1(C)C[C@@H](Cn2ncc3c(-c4ccc5c(c4)OC(F)(F)C(=O)N5Cc4ccc(Cl)cn4)c(C(N)=O)c(CC4CC4)nc32)CCO1]

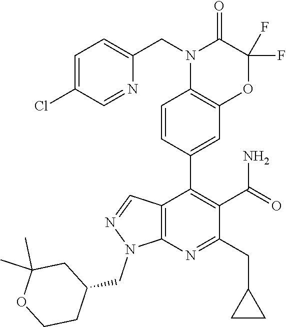 [SMILES: CC1(C)C[C@H](Cn2ncc3c(-c4ccc5c(c4)OC(F)(F)C(=O)N5Cc4ccc(Cl)cn4)c(C(N)=O)c(CC4CC4)nc32)CCO1]

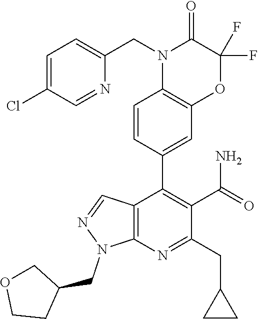 [SMILES: NC(=O)c1c(CC2CC2)nc2c(cnn2C[C@H]2CCOC2)c1-c1ccc2c(c1)OC(F)(F)C(=O)N2Cc1ccc(Cl)cn1]

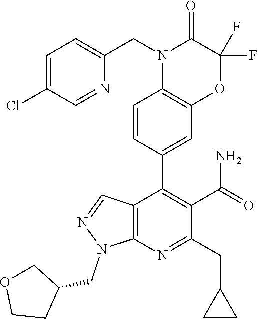 [SMILES: NC(=O)c1c(CC2CC2)nc2c(cnn2C[C@@H]2CCOC2)c1-c1ccc2c(c1)OC(F)(F)C(=O)N2Cc1ccc(Cl)cn1]

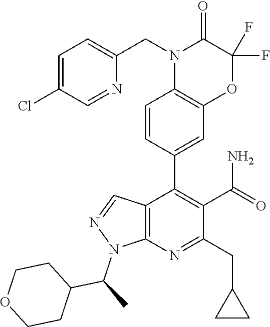 [SMILES: C[C@@H](C1CCOCC1)n1ncc2c(-c3ccc4c(c3)OC(F)(F)C(=O)N4Cc3ccc(Cl)cn3)c(C(N)=O)c(CC3CC3)nc21]

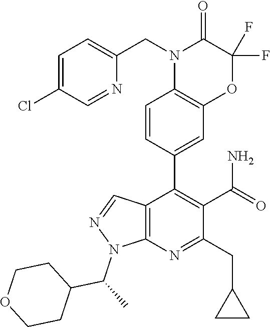 [SMILES: C[C@H](C1CCOCC1)n1ncc2c(-c3ccc4c(c3)OC(F)(F)C(=O)N4Cc3ccc(Cl)cn3)c(C(N)=O)c(CC3CC3)nc21]

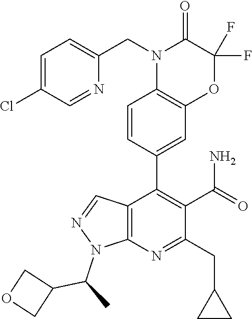 [SMILES: C[C@@H](C1COC1)n1ncc2c(-c3ccc4c(c3)OC(F)(F)C(=O)N4Cc3ccc(Cl)cn3)c(C(N)=O)c(CC3CC3)nc21]

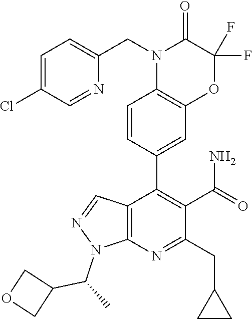 [SMILES: C[C@H](C1COC1)n1ncc2c(-c3ccc4c(c3)OC(F)(F)C(=O)N4Cc3ccc(Cl)cn3)c(C(N)=O)c(CC3CC3)nc21]

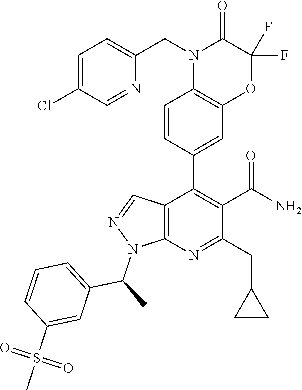 [SMILES: C[C@@H](c1cccc(S(C)(=O)=O)c1)n1ncc2c(-c3ccc4c(c3)OC(F)(F)C(=O)N4Cc3ccc(Cl)cn3)c(C(N)=O)c(CC3CC3)nc21]

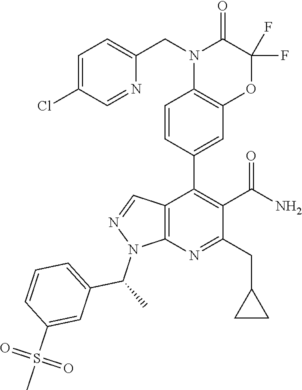 [SMILES: C[C@H](c1cccc(S(C)(=O)=O)c1)n1ncc2c(-c3ccc4c(c3)OC(F)(F)C(=O)N4Cc3ccc(Cl)cn3)c(C(N)=O)c(CC3CC3)nc21]

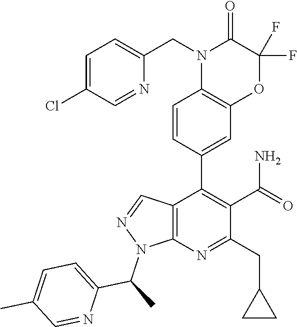 [SMILES: Cc1ccc([C@H](C)n2ncc3c(-c4ccc5c(c4)OC(F)(F)C(=O)N5Cc4ccc(Cl)cn4)c(C(N)=O)c(CC4CC4)nc32)nc1]

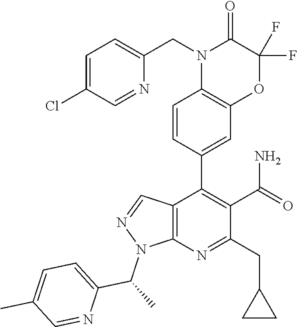 [SMILES: Cc1ccc([C@@H](C)n2ncc3c(-c4ccc5c(c4)OC(F)(F)C(=O)N5Cc4ccc(Cl)cn4)c(C(N)=O)c(CC4CC4)nc32)nc1]

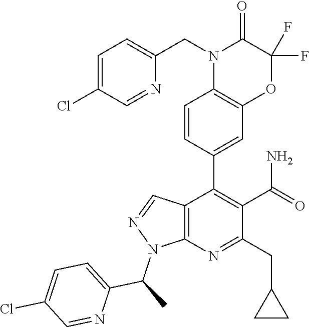 [SMILES: C[C@@H](c1ccc(Cl)cn1)n1ncc2c(-c3ccc4c(c3)OC(F)(F)C(=O)N4Cc3ccc(Cl)cn3)c(C(N)=O)c(CC3CC3)nc21]

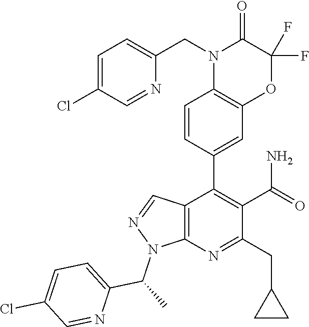 [SMILES: C[C@H](c1ccc(Cl)cn1)n1ncc2c(-c3ccc4c(c3)OC(F)(F)C(=O)N4Cc3ccc(Cl)cn3)c(C(N)=O)c(CC3CC3)nc21]

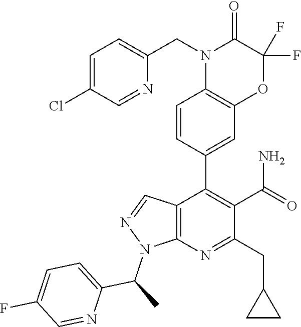 [SMILES: C[C@@H](c1ccc(F)cn1)n1ncc2c(-c3ccc4c(c3)OC(F)(F)C(=O)N4Cc3ccc(Cl)cn3)c(C(N)=O)c(CC3CC3)nc21]

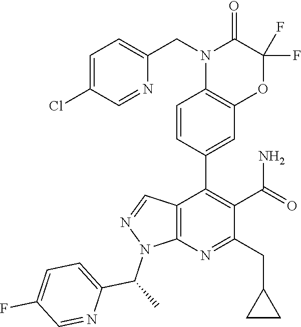 [SMILES: C[C@H](c1ccc(F)cn1)n1ncc2c(-c3ccc4c(c3)OC(F)(F)C(=O)N4Cc3ccc(Cl)cn3)c(C(N)=O)c(CC3CC3)nc21]

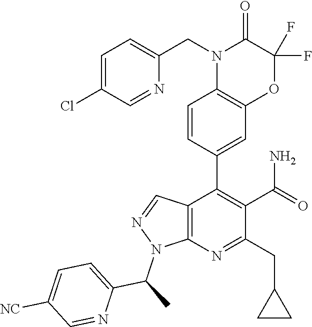 [SMILES: C[C@@H](c1ccc(C#N)cn1)n1ncc2c(-c3ccc4c(c3)OC(F)(F)C(=O)N4Cc3ccc(Cl)cn3)c(C(N)=O)c(CC3CC3)nc21]

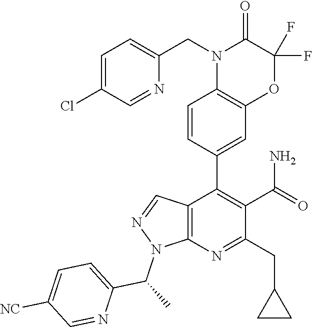 [SMILES: C[C@H](c1ccc(C#N)cn1)n1ncc2c(-c3ccc4c(c3)OC(F)(F)C(=O)N4Cc3ccc(Cl)cn3)c(C(N)=O)c(CC3CC3)nc21]

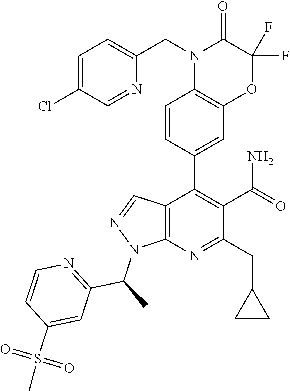 [SMILES: C[C@@H](c1cc(S(C)(=O)=O)ccn1)n1ncc2c(-c3ccc4c(c3)OC(F)(F)C(=O)N4Cc3ccc(Cl)cn3)c(C(N)=O)c(CC3CC3)nc21]

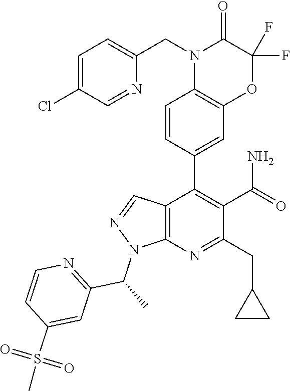 [SMILES: C[C@H](c1cc(S(C)(=O)=O)ccn1)n1ncc2c(-c3ccc4c(c3)OC(F)(F)C(=O)N4Cc3ccc(Cl)cn3)c(C(N)=O)c(CC3CC3)nc21]

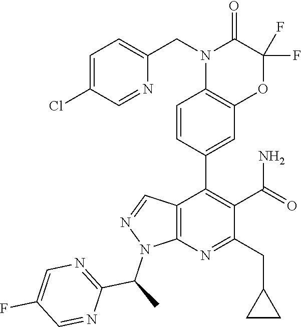 [SMILES: C[C@@H](c1ncc(F)cn1)n1ncc2c(-c3ccc4c(c3)OC(F)(F)C(=O)N4Cc3ccc(Cl)cn3)c(C(N)=O)c(CC3CC3)nc21]

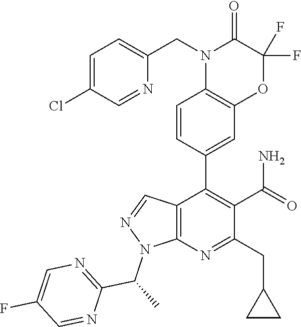 [SMILES: C[C@H](c1ncc(F)cn1)n1ncc2c(-c3ccc4c(c3)OC(F)(F)C(=O)N4Cc3ccc(Cl)cn3)c(C(N)=O)c(CC3CC3)nc21]

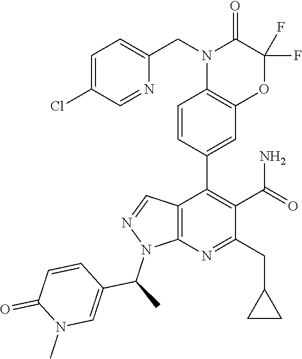 [SMILES: C[C@@H](c1ccc(=O)n(C)c1)n1ncc2c(-c3ccc4c(c3)OC(F)(F)C(=O)N4Cc3ccc(Cl)cn3)c(C(N)=O)c(CC3CC3)nc21]

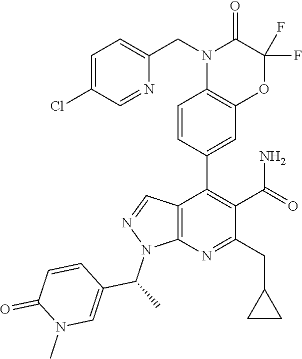 [SMILES: C[C@H](c1ccc(=O)n(C)c1)n1ncc2c(-c3ccc4c(c3)OC(F)(F)C(=O)N4Cc3ccc(Cl)cn3)c(C(N)=O)c(CC3CC3)nc21]

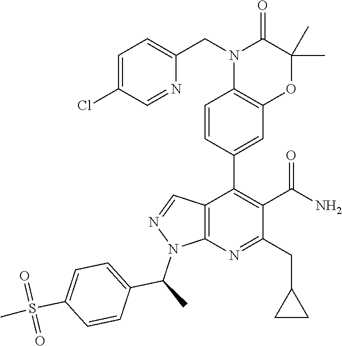

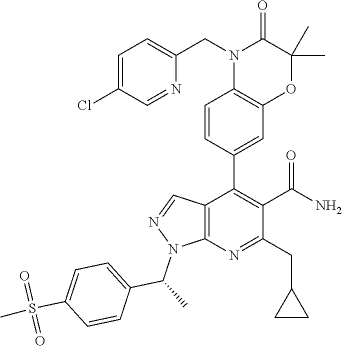

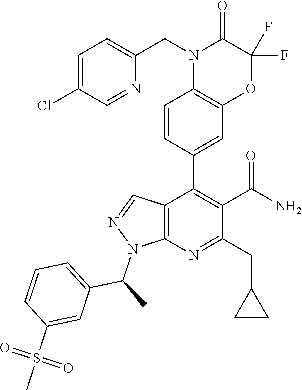 [SMILES: C[C@@H](c1cccc(S(C)(=O)=O)c1)n1ncc2c(-c3ccc4c(c3)OC(F)(F)C(=O)N4Cc3ccc(Cl)cn3)c(C(N)=O)c(CC3CC3)nc21]

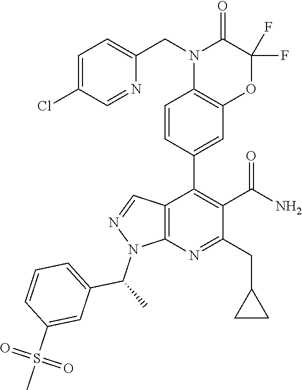 [SMILES: C[C@H](c1cccc(S(C)(=O)=O)c1)n1ncc2c(-c3ccc4c(c3)OC(F)(F)C(=O)N4Cc3ccc(Cl)cn3)c(C(N)=O)c(CC3CC3)nc21]

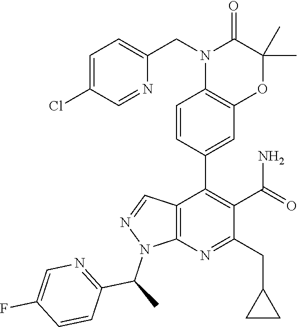 [SMILES: C[C@@H](c1ccc(F)cn1)n1ncc2c(-c3ccc4c(c3)OC(C)(C)C(=O)N4Cc3ccc(Cl)cn3)c(C(N)=O)c(CC3CC3)nc21]

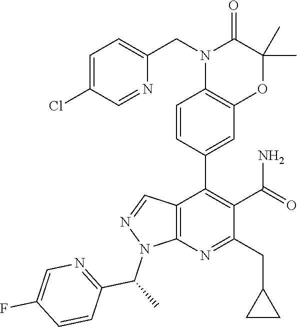 [SMILES: C[C@H](c1ccc(F)cn1)n1ncc2c(-c3ccc4c(c3)OC(C)(C)C(=O)N4Cc3ccc(Cl)cn3)c(C(N)=O)c(CC3CC3)nc21]

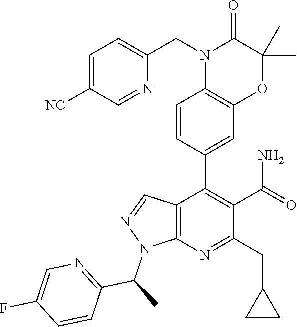 [SMILES: C[C@@H](c1ccc(F)cn1)n1ncc2c(-c3ccc4c(c3)OC(C)(C)C(=O)N4Cc3ccc(C#N)cn3)c(C(N)=O)c(CC3CC3)nc21]

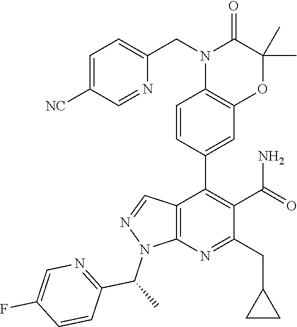 [SMILES: C[C@H](c1ccc(F)cn1)n1ncc2c(-c3ccc4c(c3)OC(C)(C)C(=O)N4Cc3ccc(C#N)cn3)c(C(N)=O)c(CC3CC3)nc21]

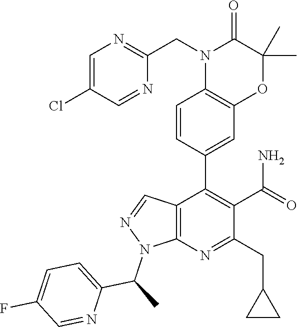 [SMILES: C[C@@H](c1ccc(F)cn1)n1ncc2c(-c3ccc4c(c3)OC(C)(C)C(=O)N4Cc3ncc(Cl)cn3)c(C(N)=O)c(CC3CC3)nc21]

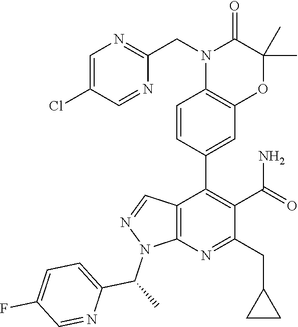 [SMILES: C[C@H](c1ccc(F)cn1)n1ncc2c(-c3ccc4c(c3)OC(C)(C)C(=O)N4Cc3ncc(Cl)cn3)c(C(N)=O)c(CC3CC3)nc21]

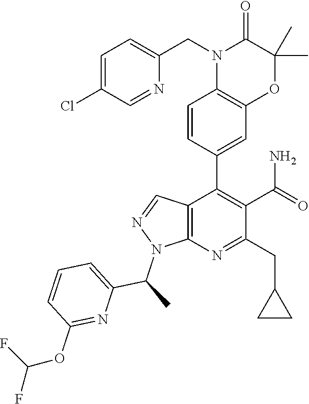 [SMILES: C[C@@H](c1cccc(OC(F)F)n1)n1ncc2c(-c3ccc4c(c3)OC(C)(C)C(=O)N4Cc3ccc(Cl)cn3)c(C(N)=O)c(CC3CC3)nc21]

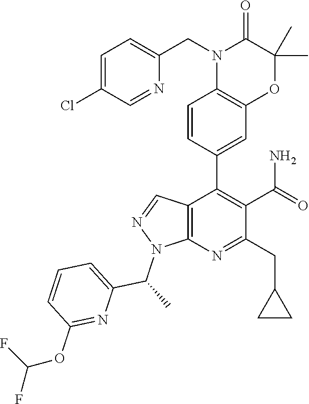 [SMILES: C[C@H](c1cccc(OC(F)F)n1)n1ncc2c(-c3ccc4c(c3)OC(C)(C)C(=O)N4Cc3ccc(Cl)cn3)c(C(N)=O)c(CC3CC3)nc21]

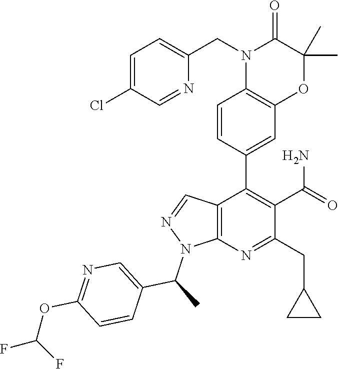 [SMILES: C[C@@H](c1ccc(OC(F)F)nc1)n1ncc2c(-c3ccc4c(c3)OC(C)(C)C(=O)N4Cc3ccc(Cl)cn3)c(C(N)=O)c(CC3CC3)nc21]

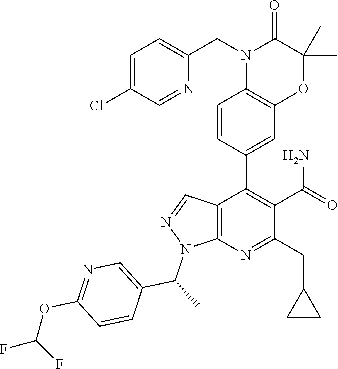 [SMILES: C[C@H](c1ccc(OC(F)F)nc1)n1ncc2c(-c3ccc4c(c3)OC(C)(C)C(=O)N4Cc3ccc(Cl)cn3)c(C(N)=O)c(CC3CC3)nc21]

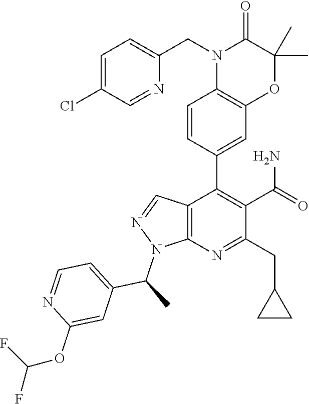 [SMILES: C[C@@H](c1ccnc(OC(F)F)c1)n1ncc2c(-c3ccc4c(c3)OC(C)(C)C(=O)N4Cc3ccc(Cl)cn3)c(C(N)=O)c(CC3CC3)nc21]

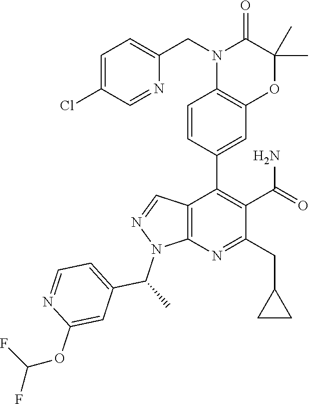 [SMILES: C[C@H](c1ccnc(OC(F)F)c1)n1ncc2c(-c3ccc4c(c3)OC(C)(C)C(=O)N4Cc3ccc(Cl)cn3)c(C(N)=O)c(CC3CC3)nc21]

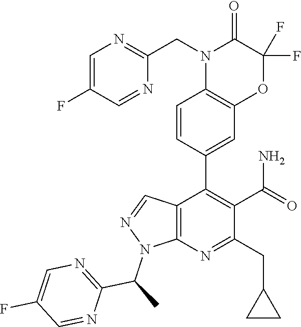 [SMILES: C[C@@H](c1ncc(F)cn1)n1ncc2c(-c3ccc4c(c3)OC(F)(F)C(=O)N4Cc3ncc(F)cn3)c(C(N)=O)c(CC3CC3)nc21]

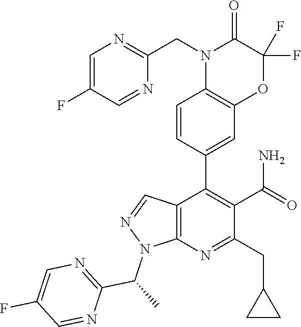 [SMILES: C[C@H](c1ncc(F)cn1)n1ncc2c(-c3ccc4c(c3)OC(F)(F)C(=O)N4Cc3ncc(F)cn3)c(C(N)=O)c(CC3CC3)nc21]

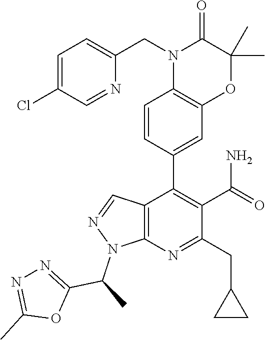

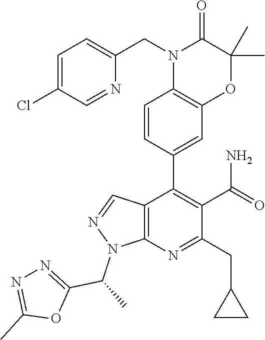

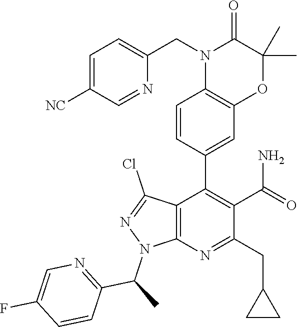 [SMILES: C[C@@H](c1ccc(F)cn1)n1nc(Cl)c2c(-c3ccc4c(c3)OC(C)(C)C(=O)N4Cc3ccc(C#N)cn3)c(C(N)=O)c(CC3CC3)nc21]

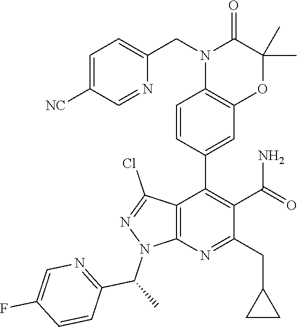 [SMILES: C[C@H](c1ccc(F)cn1)n1nc(Cl)c2c(-c3ccc4c(c3)OC(C)(C)C(=O)N4Cc3ccc(C#N)cn3)c(C(N)=O)c(CC3CC3)nc21]

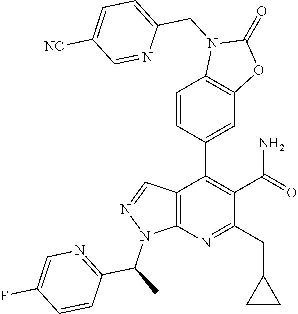 [SMILES: C[C@@H](c1ccc(F)cn1)n1ncc2c(-c3ccc4c(c3)oc(=O)n4Cc3ccc(C#N)cn3)c(C(N)=O)c(CC3CC3)nc21]

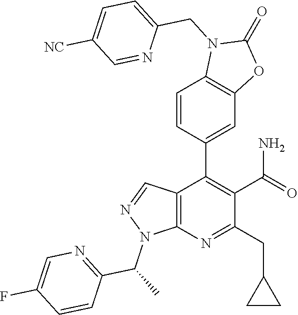 [SMILES: C[C@H](c1ccc(F)cn1)n1ncc2c(-c3ccc4c(c3)oc(=O)n4Cc3ccc(C#N)cn3)c(C(N)=O)c(CC3CC3)nc21]

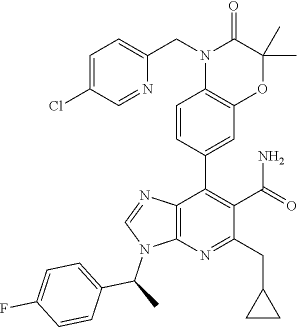 [SMILES: C[C@@H](c1ccc(F)cc1)n1cnc2c(-c3ccc4c(c3)OC(C)(C)C(=O)N4Cc3ccc(Cl)cn3)c(C(N)=O)c(CC3CC3)nc21]

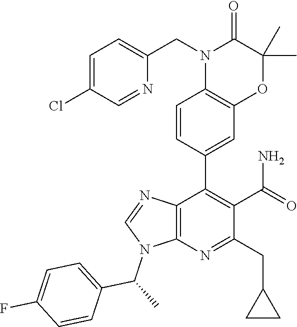 [SMILES: C[C@H](c1ccc(F)cc1)n1cnc2c(-c3ccc4c(c3)OC(C)(C)C(=O)N4Cc3ccc(Cl)cn3)c(C(N)=O)c(CC3CC3)nc21]

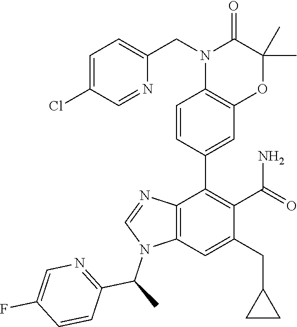 [SMILES: C[C@@H](c1ccc(F)cn1)n1cnc2c(-c3ccc4c(c3)OC(C)(C)C(=O)N4Cc3ccc(Cl)cn3)c(C(N)=O)c(CC3CC3)cc21]

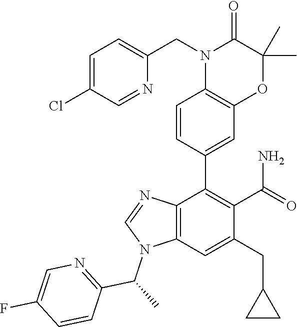 [SMILES: C[C@H](c1ccc(F)cn1)n1cnc2c(-c3ccc4c(c3)OC(C)(C)C(=O)N4Cc3ccc(Cl)cn3)c(C(N)=O)c(CC3CC3)cc21]

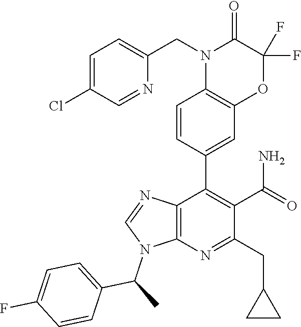 [SMILES: C[C@@H](c1ccc(F)cc1)n1cnc2c(-c3ccc4c(c3)OC(F)(F)C(=O)N4Cc3ccc(Cl)cn3)c(C(N)=O)c(CC3CC3)nc21]

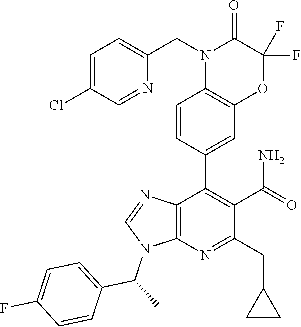 [SMILES: C[C@H](c1ccc(F)cc1)n1cnc2c(-c3ccc4c(c3)OC(F)(F)C(=O)N4Cc3ccc(Cl)cn3)c(C(N)=O)c(CC3CC3)nc21]

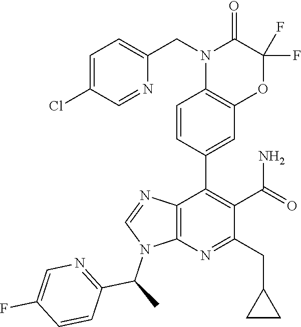 [SMILES: C[C@@H](c1ccc(F)cn1)n1cnc2c(-c3ccc4c(c3)OC(F)(F)C(=O)N4Cc3ccc(Cl)cn3)c(C(N)=O)c(CC3CC3)nc21]

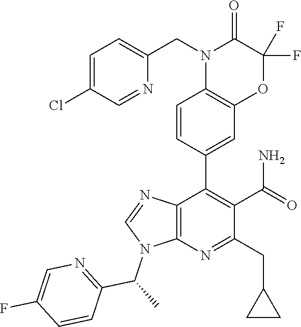 [SMILES: C[C@H](c1ccc(F)cn1)n1cnc2c(-c3ccc4c(c3)OC(F)(F)C(=O)N4Cc3ccc(Cl)cn3)c(C(N)=O)c(CC3CC3)nc21]

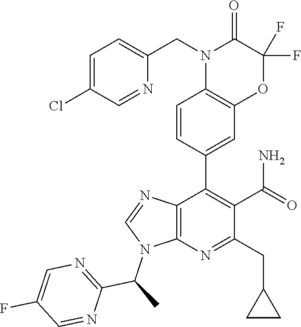 [SMILES: C[C@@H](c1ncc(F)cn1)n1cnc2c(-c3ccc4c(c3)OC(F)(F)C(=O)N4Cc3ccc(Cl)cn3)c(C(N)=O)c(CC3CC3)nc21]

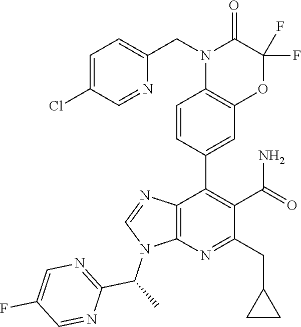 [SMILES: C[C@H](c1ncc(F)cn1)n1cnc2c(-c3ccc4c(c3)OC(F)(F)C(=O)N4Cc3ccc(Cl)cn3)c(C(N)=O)c(CC3CC3)nc21]

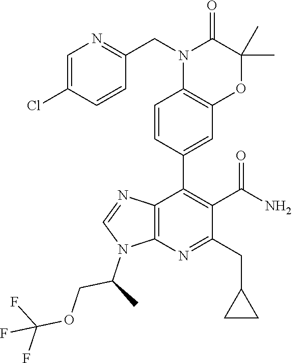 [SMILES: C[C@@H](COC(F)(F)F)n1cnc2c(-c3ccc4c(c3)OC(C)(C)C(=O)N4Cc3ccc(Cl)cn3)c(C(N)=O)c(CC3CC3)nc21]

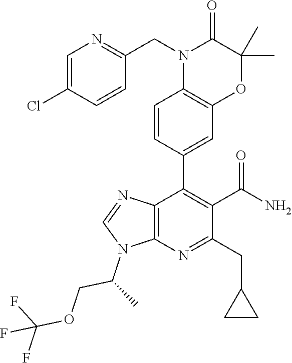 [SMILES: C[C@H](COC(F)(F)F)n1cnc2c(-c3ccc4c(c3)OC(C)(C)C(=O)N4Cc3ccc(Cl)cn3)c(C(N)=O)c(CC3CC3)nc21]

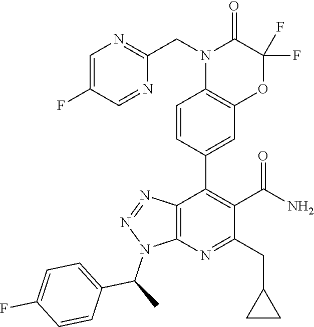 [SMILES: C[C@@H](c1ccc(F)cc1)n1nnc2c(-c3ccc4c(c3)OC(F)(F)C(=O)N4Cc3ncc(F)cn3)c(C(N)=O)c(CC3CC3)nc21]

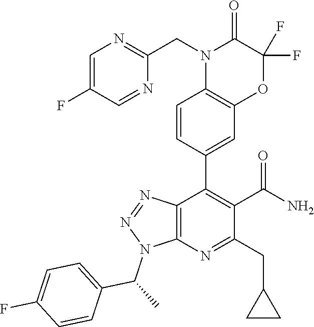 [SMILES: C[C@H](c1ccc(F)cc1)n1nnc2c(-c3ccc4c(c3)OC(F)(F)C(=O)N4Cc3ncc(F)cn3)c(C(N)=O)c(CC3CC3)nc21]

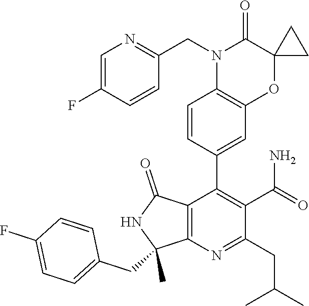 [SMILES: CC(C)Cc1nc2c(c(-c3ccc4c(c3)OC3(CC3)C(=O)N4Cc3ccc(F)cn3)c1C(N)=O)C(=O)N[C@@]2(C)Cc1ccc(F)cc1]

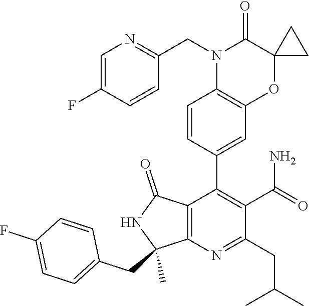 [SMILES: CC(C)Cc1nc2c(c(-c3ccc4c(c3)OC3(CC3)C(=O)N4Cc3ccc(F)cn3)c1C(N)=O)C(=O)N[C@]2(C)Cc1ccc(F)cc1]

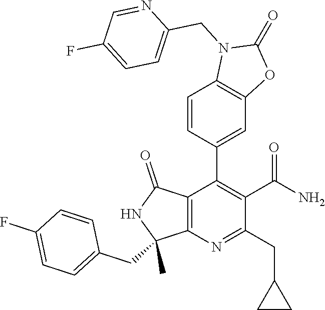 [SMILES: C[C@@]1(Cc2ccc(F)cc2)NC(=O)c2c1nc(CC1CC1)c(C(N)=O)c2-c1ccc2c(c1)oc(=O)n2Cc1ccc(F)cn1]

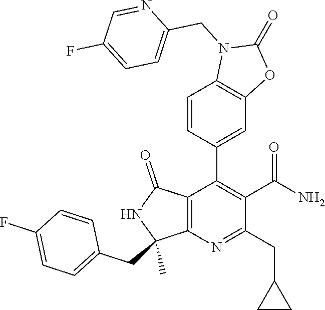 [SMILES: C[C@]1(Cc2ccc(F)cc2)NC(=O)c2c1nc(CC1CC1)c(C(N)=O)c2-c1ccc2c(c1)oc(=O)n2Cc1ccc(F)cn1]

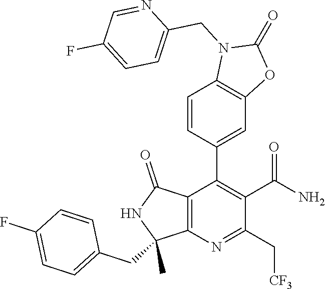 [SMILES: C[C@@]1(Cc2ccc(F)cc2)NC(=O)c2c1nc(CC(F)(F)F)c(C(N)=O)c2-c1ccc2c(c1)oc(=O)n2Cc1ccc(F)cn1]

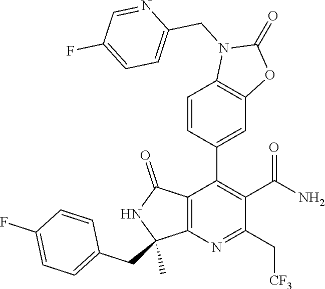 [SMILES: C[C@]1(Cc2ccc(F)cc2)NC(=O)c2c1nc(CC(F)(F)F)c(C(N)=O)c2-c1ccc2c(c1)oc(=O)n2Cc1ccc(F)cn1]

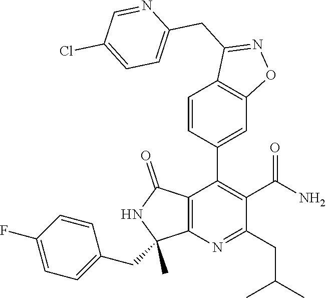 [SMILES: CC(C)Cc1nc2c(c(-c3ccc4c(Cc5ccc(Cl)cn5)noc4c3)c1C(N)=O)C(=O)N[C@@]2(C)Cc1ccc(F)cc1]

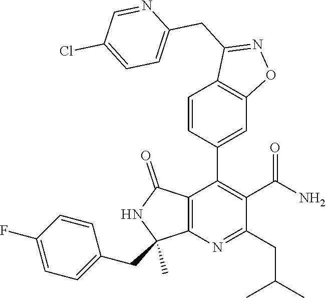 [SMILES: CC(C)Cc1nc2c(c(-c3ccc4c(Cc5ccc(Cl)cn5)noc4c3)c1C(N)=O)C(=O)N[C@]2(C)Cc1ccc(F)cc1]

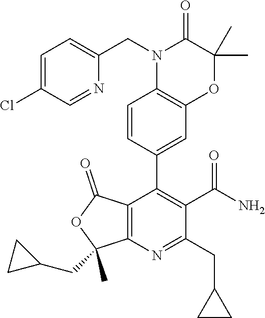 [SMILES: CC1(C)Oc2cc(-c3c(C(N)=O)c(CC4CC4)nc4c3C(=O)O[C@@]4(C)CC3CC3)ccc2N(Cc2ccc(Cl)cn2)C1=O]

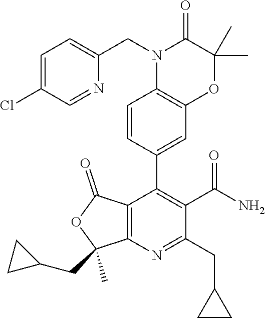 [SMILES: CC1(C)Oc2cc(-c3c(C(N)=O)c(CC4CC4)nc4c3C(=O)O[C@]4(C)CC3CC3)ccc2N(Cc2ccc(Cl)cn2)C1=O]

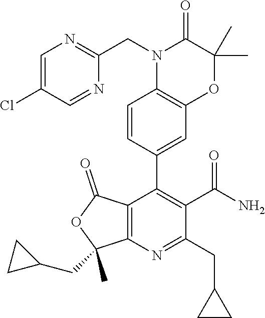 [SMILES: CC1(C)Oc2cc(-c3c(C(N)=O)c(CC4CC4)nc4c3C(=O)O[C@@]4(C)CC3CC3)ccc2N(Cc2ncc(Cl)cn2)C1=O]

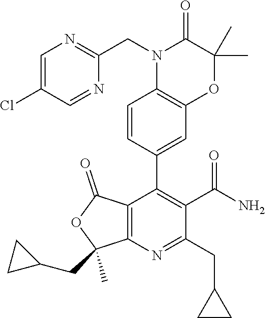 [SMILES: CC1(C)Oc2cc(-c3c(C(N)=O)c(CC4CC4)nc4c3C(=O)O[C@]4(C)CC3CC3)ccc2N(Cc2ncc(Cl)cn2)C1=O]

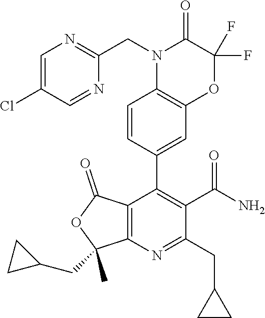 [SMILES: C[C@@]1(CC2CC2)OC(=O)c2c1nc(CC1CC1)c(C(N)=O)c2-c1ccc2c(c1)OC(F)(F)C(=O)N2Cc1ncc(Cl)cn1]

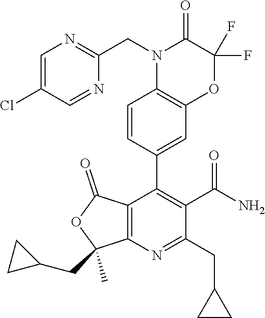 [SMILES: C[C@]1(CC2CC2)OC(=O)c2c1nc(CC1CC1)c(C(N)=O)c2-c1ccc2c(c1)OC(F)(F)C(=O)N2Cc1ncc(Cl)cn1]

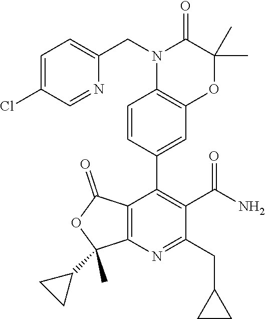 [SMILES: CC1(C)Oc2cc(-c3c(C(N)=O)c(CC4CC4)nc4c3C(=O)O[C@@]4(C)C3CC3)ccc2N(Cc2ccc(Cl)cn2)C1=O]

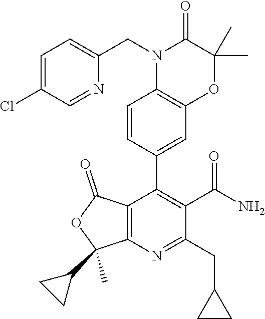 [SMILES: CC1(C)Oc2cc(-c3c(C(N)=O)c(CC4CC4)nc4c3C(=O)O[C@]4(C)C3CC3)ccc2N(Cc2ccc(Cl)cn2)C1=O]

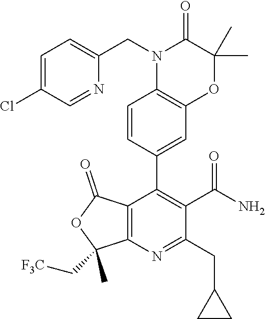 [SMILES: CC1(C)Oc2cc(-c3c(C(N)=O)c(CC4CC4)nc4c3C(=O)O[C@@]4(C)CC(F)(F)F)ccc2N(Cc2ccc(Cl)cn2)C1=O]

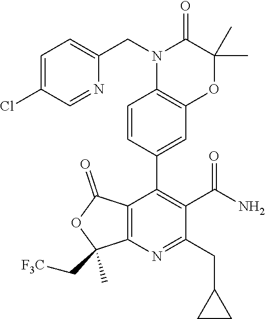 [SMILES: CC1(C)Oc2cc(-c3c(C(N)=O)c(CC4CC4)nc4c3C(=O)O[C@]4(C)CC(F)(F)F)ccc2N(Cc2ccc(Cl)cn2)C1=O]

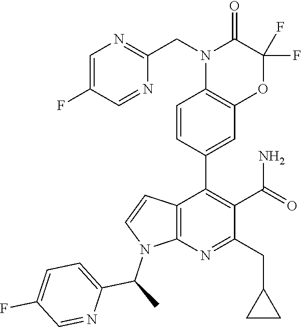 [SMILES: C[C@@H](c1ccc(F)cn1)n1ccc2c(-c3ccc4c(c3)OC(F)(F)C(=O)N4Cc3ncc(F)cn3)c(C(N)=O)c(CC3CC3)nc21]

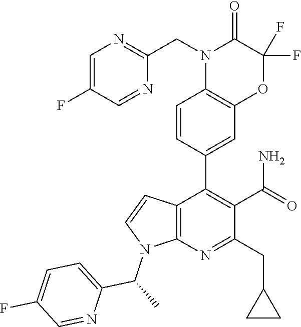 [SMILES: C[C@H](c1ccc(F)cn1)n1ccc2c(-c3ccc4c(c3)OC(F)(F)C(=O)N4Cc3ncc(F)cn3)c(C(N)=O)c(CC3CC3)nc21]

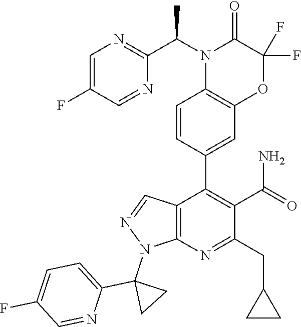 [SMILES: C[C@H](c1ncc(F)cn1)N1C(=O)C(F)(F)Oc2cc(-c3c(C(N)=O)c(CC4CC4)nc4c3cnn4C3(c4ccc(F)cn4)CC3)ccc21]

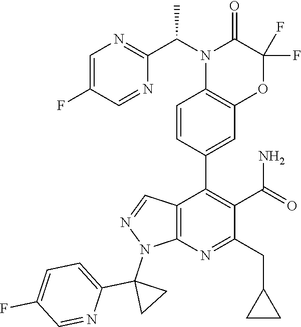 [SMILES: C[C@@H](c1ncc(F)cn1)N1C(=O)C(F)(F)Oc2cc(-c3c(C(N)=O)c(CC4CC4)nc4c3cnn4C3(c4ccc(F)cn4)CC3)ccc21]

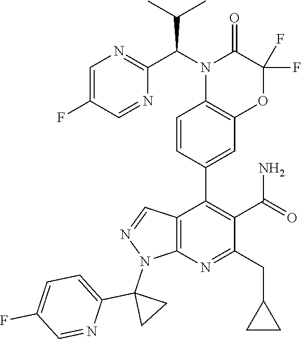 [SMILES: CC(C)[C@H](c1ncc(F)cn1)N1C(=O)C(F)(F)Oc2cc(-c3c(C(N)=O)c(CC4CC4)nc4c3cnn4C3(c4ccc(F)cn4)CC3)ccc21]

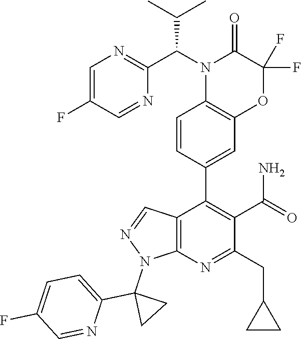 [SMILES: CC(C)[C@@H](c1ncc(F)cn1)N1C(=O)C(F)(F)Oc2cc(-c3c(C(N)=O)c(CC4CC4)nc4c3cnn4C3(c4ccc(F)cn4)CC3)ccc21]

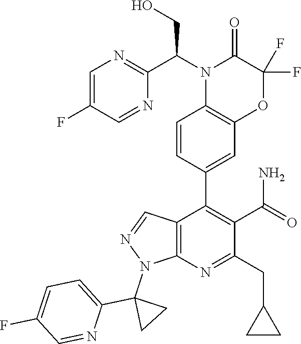 [SMILES: NC(=O)c1c(CC2CC2)nc2c(cnn2C2(c3ccc(F)cn3)CC2)c1-c1ccc2c(c1)OC(F)(F)C(=O)N2[C@H](CO)c1ncc(F)cn1]

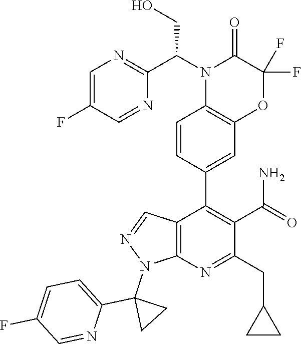 [SMILES: NC(=O)c1c(CC2CC2)nc2c(cnn2C2(c3ccc(F)cn3)CC2)c1-c1ccc2c(c1)OC(F)(F)C(=O)N2[C@@H](CO)c1ncc(F)cn1]

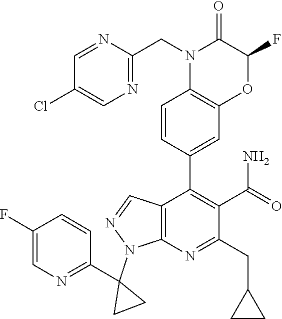 [SMILES: NC(=O)c1c(CC2CC2)nc2c(cnn2C2(c3ccc(F)cn3)CC2)c1-c1ccc2c(c1)O[C@H](F)C(=O)N2Cc1ncc(Cl)cn1]

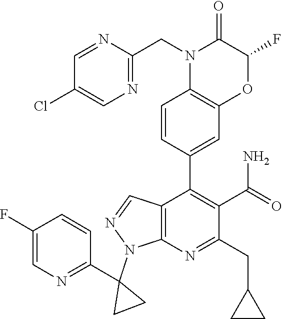 [SMILES: NC(=O)c1c(CC2CC2)nc2c(cnn2C2(c3ccc(F)cn3)CC2)c1-c1ccc2c(c1)O[C@@H](F)C(=O)N2Cc1ncc(Cl)cn1]

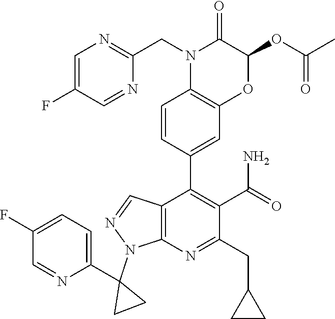 [SMILES: CC(=O)O[C@H]1Oc2cc(-c3c(C(N)=O)c(CC4CC4)nc4c3cnn4C3(c4ccc(F)cn4)CC3)ccc2N(Cc2ncc(F)cn2)C1=O]

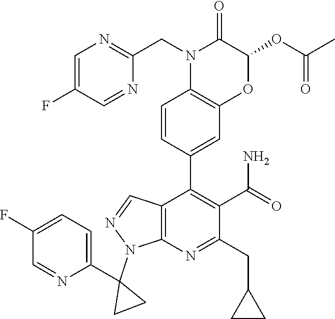 [SMILES: CC(=O)O[C@@H]1Oc2cc(-c3c(C(N)=O)c(CC4CC4)nc4c3cnn4C3(c4ccc(F)cn4)CC3)ccc2N(Cc2ncc(F)cn2)C1=O]

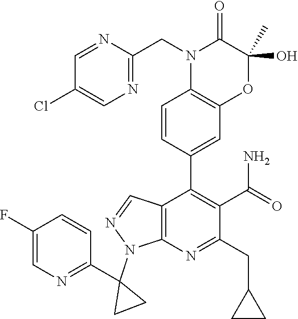 [SMILES: C[C@]1(O)Oc2cc(-c3c(C(N)=O)c(CC4CC4)nc4c3cnn4C3(c4ccc(F)cn4)CC3)ccc2N(Cc2ncc(Cl)cn2)C1=O]

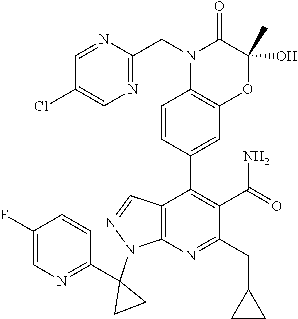 [SMILES: C[C@@]1(O)Oc2cc(-c3c(C(N)=O)c(CC4CC4)nc4c3cnn4C3(c4ccc(F)cn4)CC3)ccc2N(Cc2ncc(Cl)cn2)C1=O]

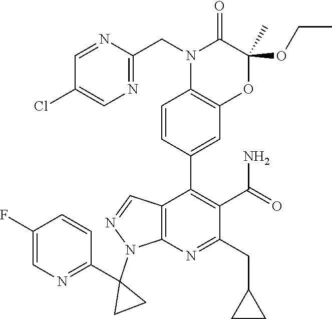 [SMILES: CCO[C@@]1(C)Oc2cc(-c3c(C(N)=O)c(CC4CC4)nc4c3cnn4C3(c4ccc(F)cn4)CC3)ccc2N(Cc2ncc(Cl)cn2)C1=O]

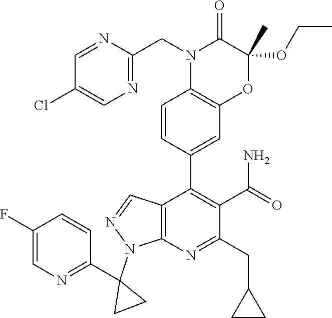 [SMILES: CCO[C@]1(C)Oc2cc(-c3c(C(N)=O)c(CC4CC4)nc4c3cnn4C3(c4ccc(F)cn4)CC3)ccc2N(Cc2ncc(Cl)cn2)C1=O]

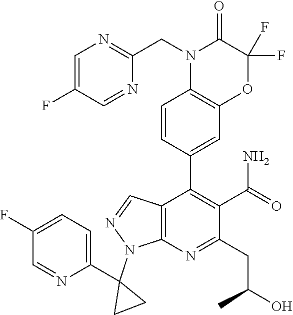 [SMILES: C[C@H](O)Cc1nc2c(cnn2C2(c3ccc(F)cn3)CC2)c(-c2ccc3c(c2)OC(F)(F)C(=O)N3Cc2ncc(F)cn2)c1C(N)=O]

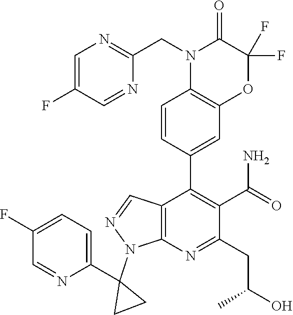 [SMILES: C[C@@H](O)Cc1nc2c(cnn2C2(c3ccc(F)cn3)CC2)c(-c2ccc3c(c2)OC(F)(F)C(=O)N3Cc2ncc(F)cn2)c1C(N)=O]

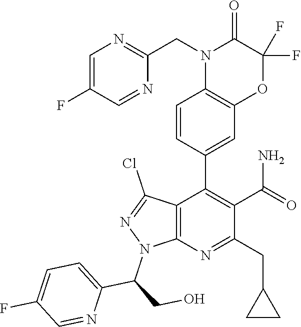 [SMILES: NC(=O)c1c(CC2CC2)nc2c(c(Cl)nn2[C@@H](CO)c2ccc(F)cn2)c1-c1ccc2c(c1)OC(F)(F)C(=O)N2Cc1ncc(F)cn1]

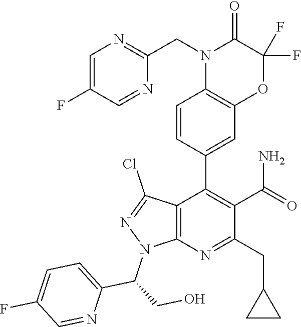 [SMILES: NC(=O)c1c(CC2CC2)nc2c(c(Cl)nn2[C@H](CO)c2ccc(F)cn2)c1-c1ccc2c(c1)OC(F)(F)C(=O)N2Cc1ncc(F)cn1]

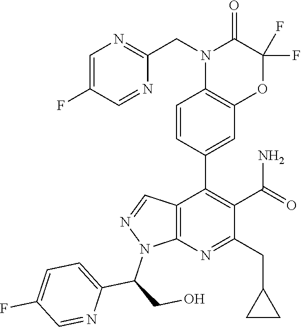 [SMILES: NC(=O)c1c(CC2CC2)nc2c(cnn2[C@@H](CO)c2ccc(F)cn2)c1-c1ccc2c(c1)OC(F)(F)C(=O)N2Cc1ncc(F)cn1]

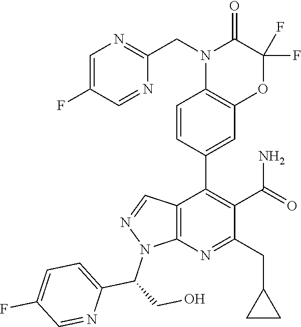 [SMILES: NC(=O)c1c(CC2CC2)nc2c(cnn2[C@H](CO)c2ccc(F)cn2)c1-c1ccc2c(c1)OC(F)(F)C(=O)N2Cc1ncc(F)cn1]

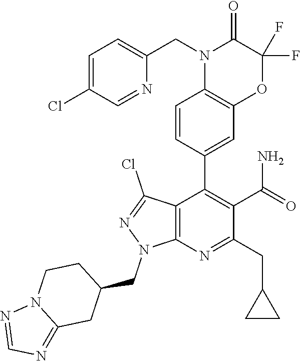 [SMILES: NC(=O)c1c(CC2CC2)nc2c(c(Cl)nn2C[C@@H]2CCn3ncnc3C2)c1-c1ccc2c(c1)OC(F)(F)C(=O)N2Cc1ccc(Cl)cn1]

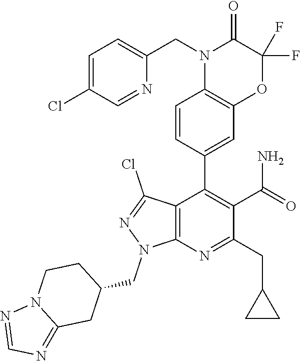 [SMILES: NC(=O)c1c(CC2CC2)nc2c(c(Cl)nn2C[C@H]2CCn3ncnc3C2)c1-c1ccc2c(c1)OC(F)(F)C(=O)N2Cc1ccc(Cl)cn1]

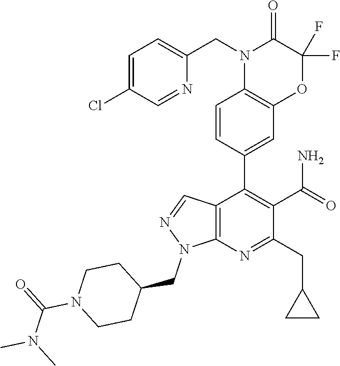

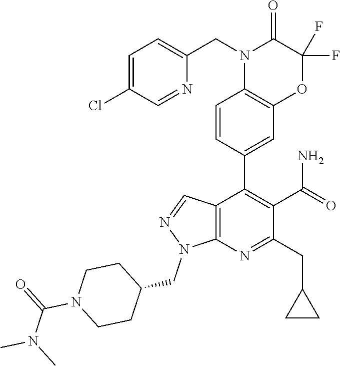

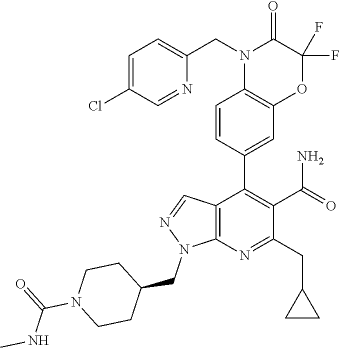

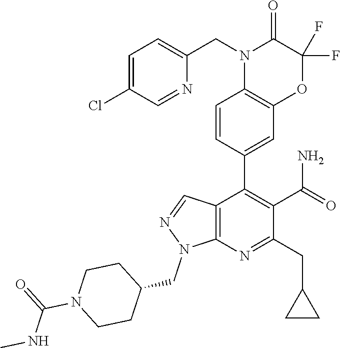

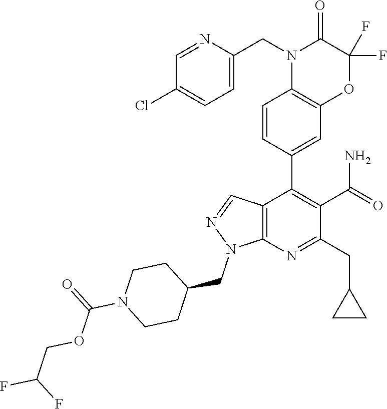

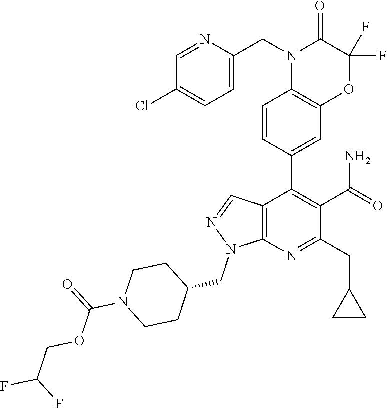

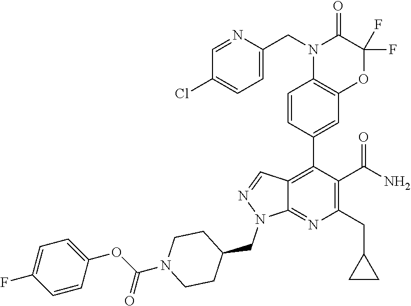

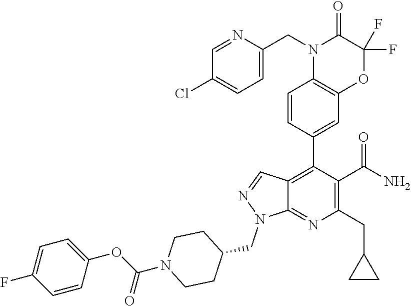

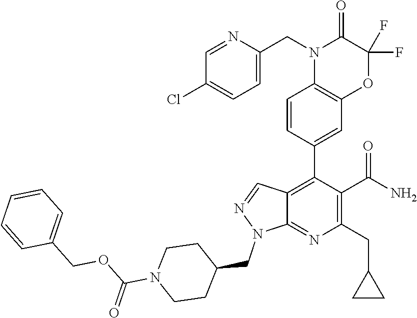

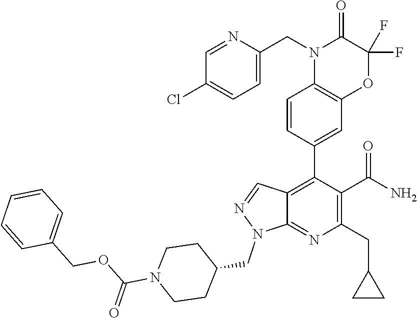

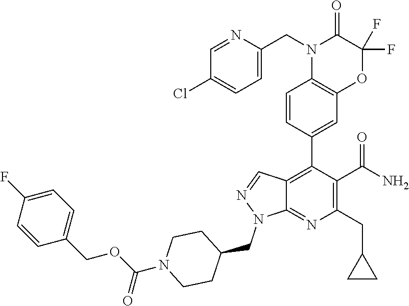

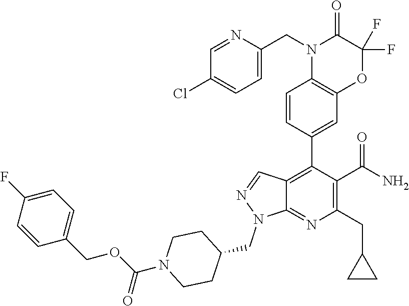

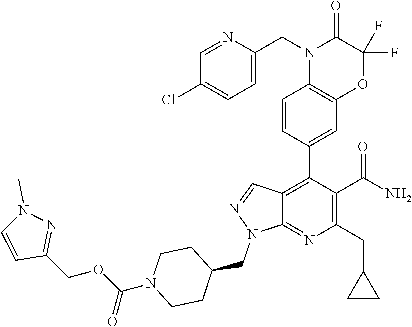

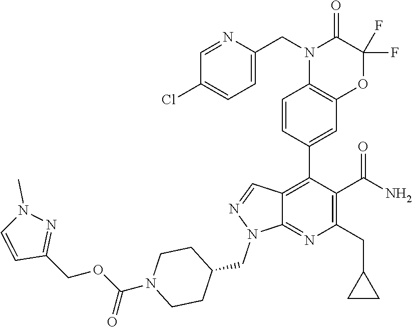

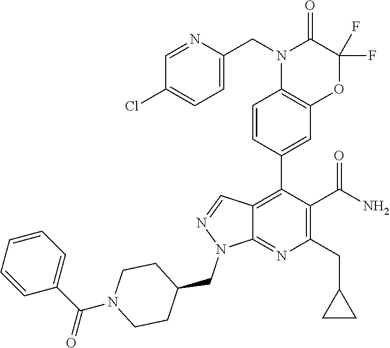

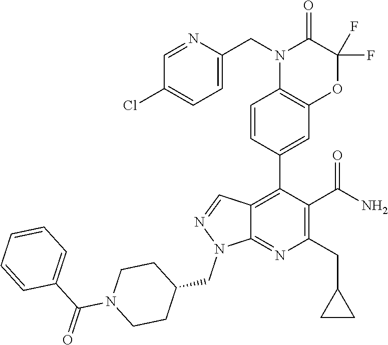

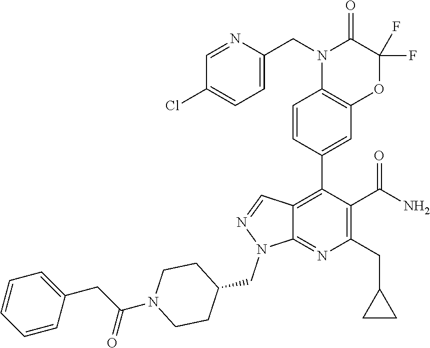

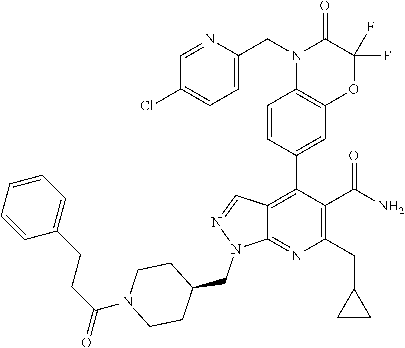

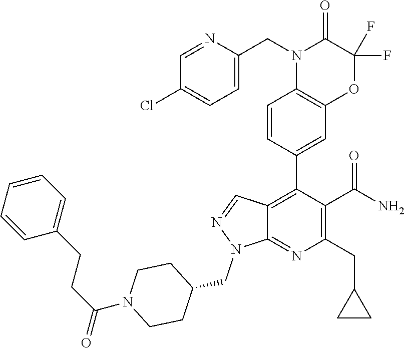

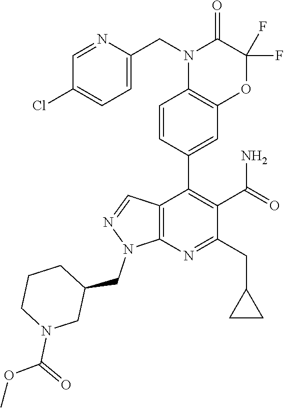 [SMILES: COC(=O)N1CCC[C@@H](Cn2ncc3c(-c4ccc5c(c4)OC(F)(F)C(=O)N5Cc4ccc(Cl)cn4)c(C(N)=O)c(CC4CC4)nc32)C1]

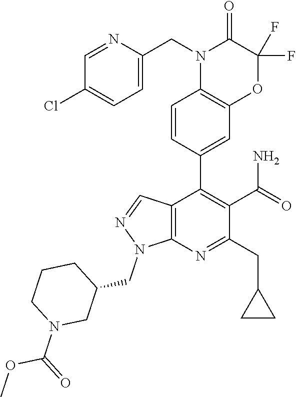 [SMILES: COC(=O)N1CCC[C@H](Cn2ncc3c(-c4ccc5c(c4)OC(F)(F)C(=O)N5Cc4ccc(Cl)cn4)c(C(N)=O)c(CC4CC4)nc32)C1]

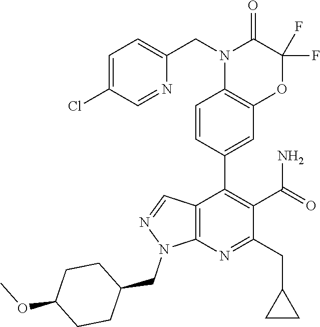 [SMILES: CO[C@H]1CC[C@@H](Cn2ncc3c(-c4ccc5c(c4)OC(F)(F)C(=O)N5Cc4ccc(Cl)cn4)c(C(N)=O)c(CC4CC4)nc32)CC1]

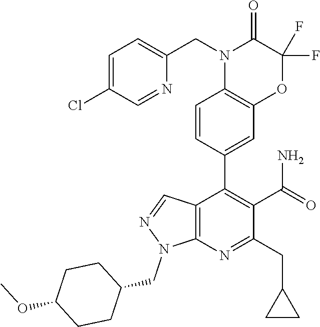 [SMILES: CO[C@H]1CC[C@@H](Cn2ncc3c(-c4ccc5c(c4)OC(F)(F)C(=O)N5Cc4ccc(Cl)cn4)c(C(N)=O)c(CC4CC4)nc32)CC1]

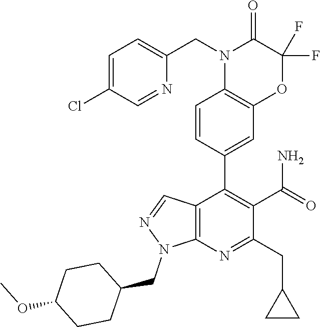 [SMILES: CO[C@H]1CC[C@H](Cn2ncc3c(-c4ccc5c(c4)OC(F)(F)C(=O)N5Cc4ccc(Cl)cn4)c(C(N)=O)c(CC4CC4)nc32)CC1]

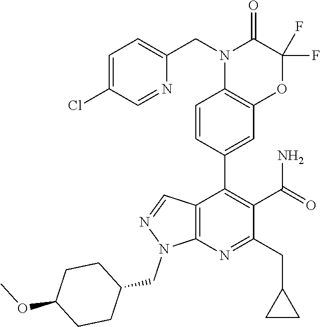 [SMILES: CO[C@H]1CC[C@H](Cn2ncc3c(-c4ccc5c(c4)OC(F)(F)C(=O)N5Cc4ccc(Cl)cn4)c(C(N)=O)c(CC4CC4)nc32)CC1]

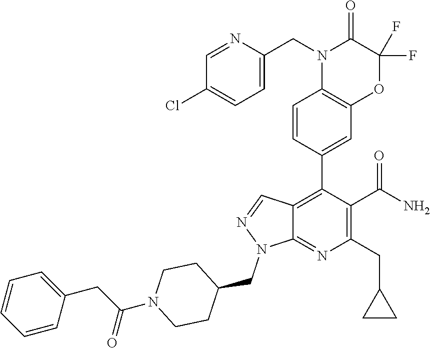

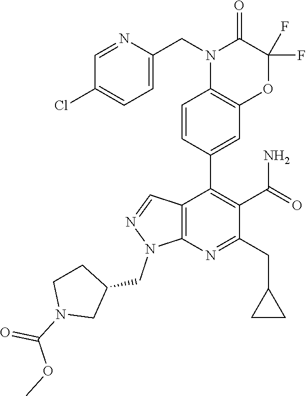 [SMILES: COC(=O)N1CC[C@H](Cn2ncc3c(-c4ccc5c(c4)OC(F)(F)C(=O)N5Cc4ccc(Cl)cn4)c(C(N)=O)c(CC4CC4)nc32)C1]

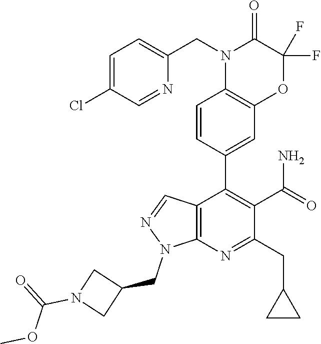

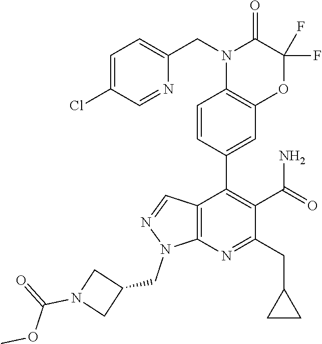

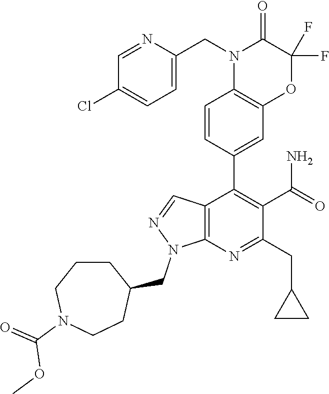 [SMILES: COC(=O)N1CCC[C@@H](Cn2ncc3c(-c4ccc5c(c4)OC(F)(F)C(=O)N5Cc4ccc(Cl)cn4)c(C(N)=O)c(CC4CC4)nc32)CC1]

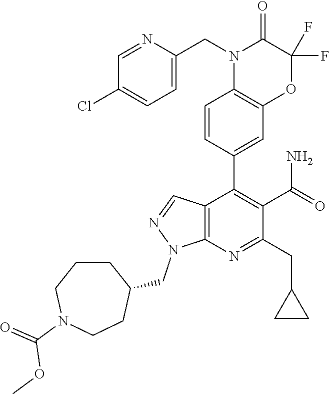 [SMILES: COC(=O)N1CCC[C@H](Cn2ncc3c(-c4ccc5c(c4)OC(F)(F)C(=O)N5Cc4ccc(Cl)cn4)c(C(N)=O)c(CC4CC4)nc32)CC1]

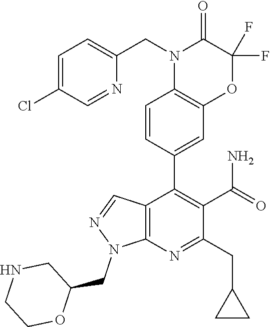 [SMILES: NC(=O)c1c(CC2CC2)nc2c(cnn2C[C@@H]2CNCCO2)c1-c1ccc2c(c1)OC(F)(F)C(=O)N2Cc1ccc(Cl)cn1]

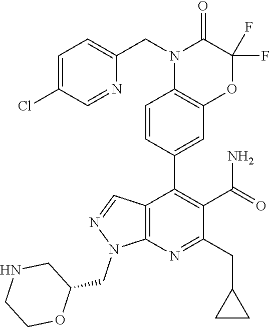 [SMILES: NC(=O)c1c(CC2CC2)nc2c(cnn2C[C@H]2CNCCO2)c1-c1ccc2c(c1)OC(F)(F)C(=O)N2Cc1ccc(Cl)cn1]

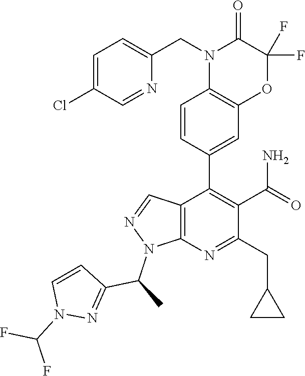 [SMILES: C[C@@H](c1ccn(C(F)F)n1)n1ncc2c(-c3ccc4c(c3)OC(F)(F)C(=O)N4Cc3ccc(Cl)cn3)c(C(N)=O)c(CC3CC3)nc21]

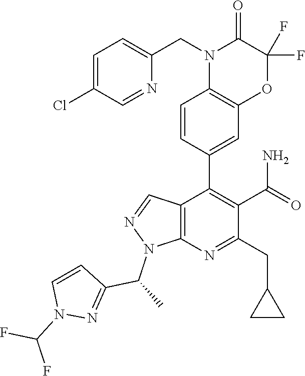 [SMILES: C[C@H](c1ccn(C(F)F)n1)n1ncc2c(-c3ccc4c(c3)OC(F)(F)C(=O)N4Cc3ccc(Cl)cn3)c(C(N)=O)c(CC3CC3)nc21]

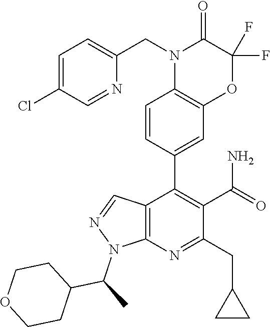 [SMILES: C[C@@H](C1CCOCC1)n1ncc2c(-c3ccc4c(c3)OC(F)(F)C(=O)N4Cc3ccc(Cl)cn3)c(C(N)=O)c(CC3CC3)nc21]

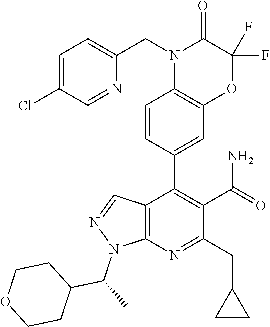 [SMILES: C[C@H](C1CCOCC1)n1ncc2c(-c3ccc4c(c3)OC(F)(F)C(=O)N4Cc3ccc(Cl)cn3)c(C(N)=O)c(CC3CC3)nc21]

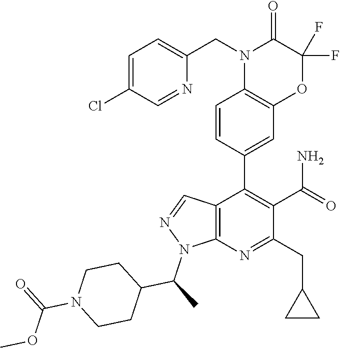 [SMILES: COC(=O)N1CCC([C@H](C)n2ncc3c(-c4ccc5c(c4)OC(F)(F)C(=O)N5Cc4ccc(Cl)cn4)c(C(N)=O)c(CC4CC4)nc32)CC1]

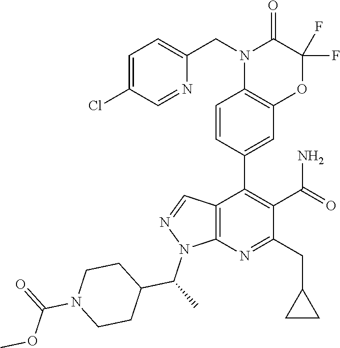 [SMILES: COC(=O)N1CCC([C@@H](C)n2ncc3c(-c4ccc5c(c4)OC(F)(F)C(=O)N5Cc4ccc(Cl)cn4)c(C(N)=O)c(CC4CC4)nc32)CC1]

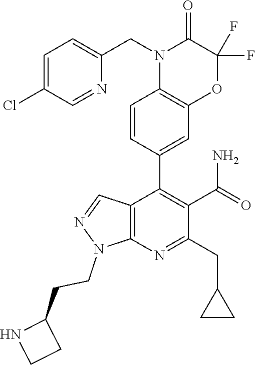 [SMILES: NC(=O)c1c(CC2CC2)nc2c(cnn2CC[C@H]2CCN2)c1-c1ccc2c(c1)OC(F)(F)C(=O)N2Cc1ccc(Cl)cn1]

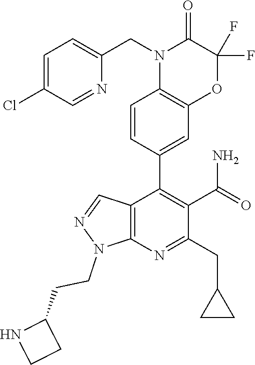 [SMILES: NC(=O)c1c(CC2CC2)nc2c(cnn2CC[C@@H]2CCN2)c1-c1ccc2c(c1)OC(F)(F)C(=O)N2Cc1ccc(Cl)cn1]

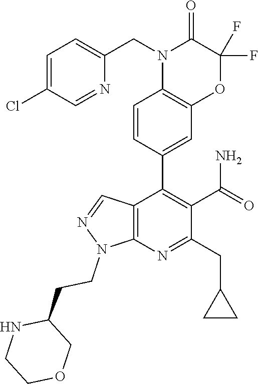 [SMILES: NC(=O)c1c(CC2CC2)nc2c(cnn2CC[C@H]2COCCN2)c1-c1ccc2c(c1)OC(F)(F)C(=O)N2Cc1ccc(Cl)cn1]

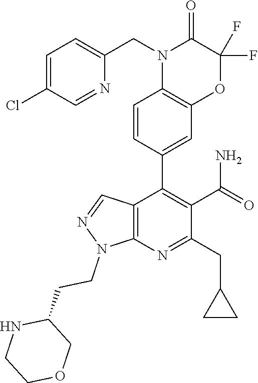 [SMILES: NC(=O)c1c(CC2CC2)nc2c(cnn2CC[C@@H]2COCCN2)c1-c1ccc2c(c1)OC(F)(F)C(=O)N2Cc1ccc(Cl)cn1]

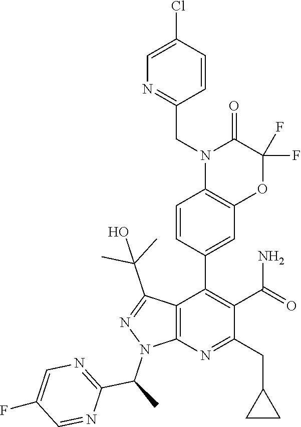 [SMILES: C[C@@H](c1ncc(F)cn1)n1nc(C(C)(C)O)c2c(-c3ccc4c(c3)OC(F)(F)C(=O)N4Cc3ccc(Cl)cn3)c(C(N)=O)c(CC3CC3)nc21]

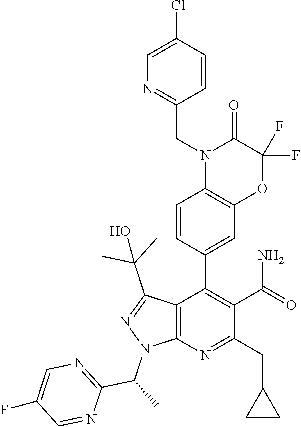 [SMILES: C[C@H](c1ncc(F)cn1)n1nc(C(C)(C)O)c2c(-c3ccc4c(c3)OC(F)(F)C(=O)N4Cc3ccc(Cl)cn3)c(C(N)=O)c(CC3CC3)nc21]

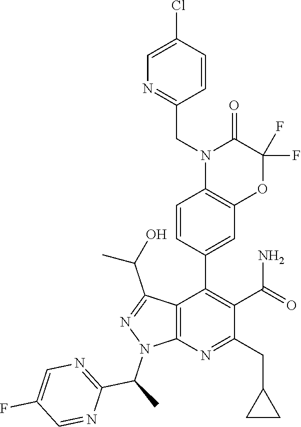 [SMILES: CC(O)c1nn([C@@H](C)c2ncc(F)cn2)c2nc(CC3CC3)c(C(N)=O)c(-c3ccc4c(c3)OC(F)(F)C(=O)N4Cc3ccc(Cl)cn3)c12]

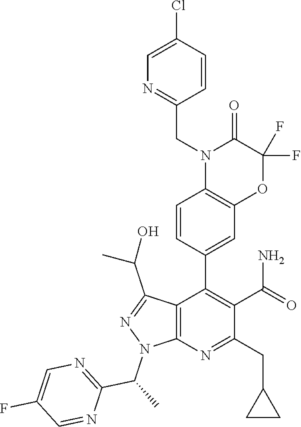 [SMILES: CC(O)c1nn([C@H](C)c2ncc(F)cn2)c2nc(CC3CC3)c(C(N)=O)c(-c3ccc4c(c3)OC(F)(F)C(=O)N4Cc3ccc(Cl)cn3)c12]

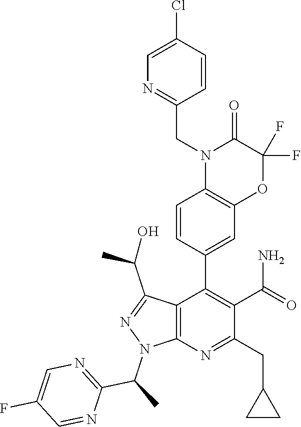 [SMILES: C[C@@H](O)c1nn([C@@H](C)c2ncc(F)cn2)c2nc(CC3CC3)c(C(N)=O)c(-c3ccc4c(c3)OC(F)(F)C(=O)N4Cc3ccc(Cl)cn3)c12]

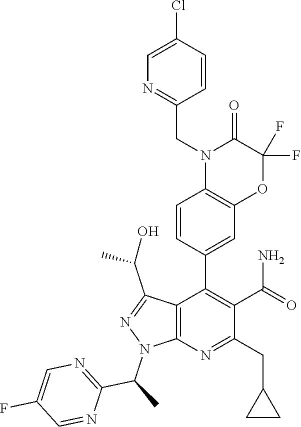 [SMILES: C[C@H](O)c1nn([C@@H](C)c2ncc(F)cn2)c2nc(CC3CC3)c(C(N)=O)c(-c3ccc4c(c3)OC(F)(F)C(=O)N4Cc3ccc(Cl)cn3)c12]

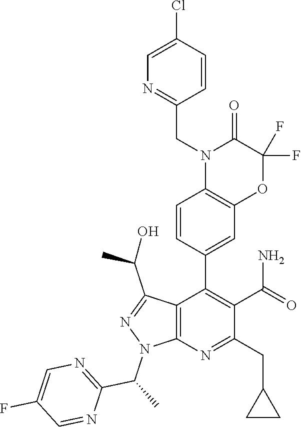 [SMILES: C[C@H](c1ncc(F)cn1)n1nc([C@@H](C)O)c2c(-c3ccc4c(c3)OC(F)(F)C(=O)N4Cc3ccc(Cl)cn3)c(C(N)=O)c(CC3CC3)nc21]

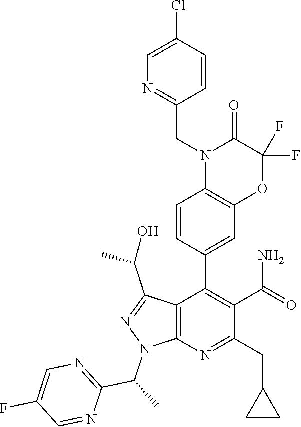 [SMILES: C[C@H](O)c1nn([C@H](C)c2ncc(F)cn2)c2nc(CC3CC3)c(C(N)=O)c(-c3ccc4c(c3)OC(F)(F)C(=O)N4Cc3ccc(Cl)cn3)c12]

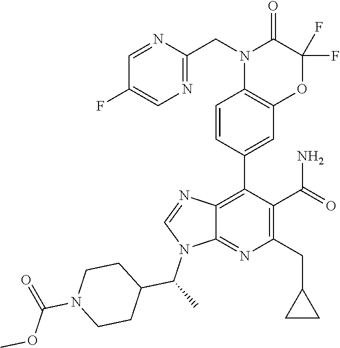 [SMILES: COC(=O)N1CCC([C@@H](C)n2cnc3c(-c4ccc5c(c4)OC(F)(F)C(=O)N5Cc4ncc(F)cn4)c(C(N)=O)c(CC4CC4)nc32)CC1]

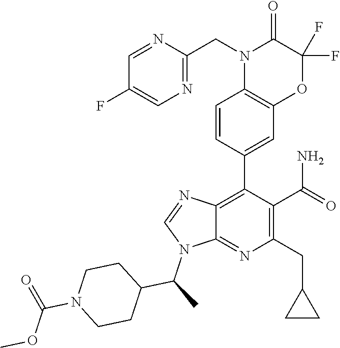 [SMILES: COC(=O)N1CCC([C@H](C)n2cnc3c(-c4ccc5c(c4)OC(F)(F)C(=O)N5Cc4ncc(F)cn4)c(C(N)=O)c(CC4CC4)nc32)CC1]

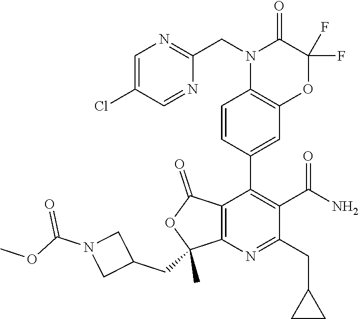 [SMILES: COC(=O)N1CC(C[C@]2(C)OC(=O)c3c2nc(CC2CC2)c(C(N)=O)c3-c2ccc3c(c2)OC(F)(F)C(=O)N3Cc2ncc(Cl)cn2)C1]

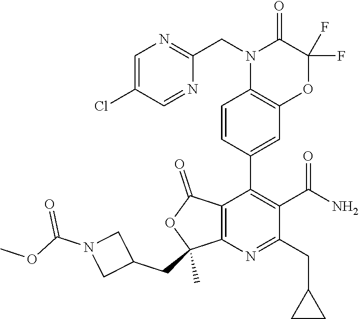 [SMILES: COC(=O)N1CC(C[C@@]2(C)OC(=O)c3c2nc(CC2CC2)c(C(N)=O)c3-c2ccc3c(c2)OC(F)(F)C(=O)N3Cc2ncc(Cl)cn2)C1]

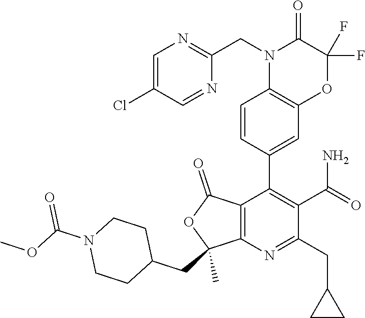 [SMILES: COC(=O)N1CCC(C[C@@]2(C)OC(=O)c3c2nc(CC2CC2)c(C(N)=O)c3-c2ccc3c(c2)OC(F)(F)C(=O)N3Cc2ncc(Cl)cn2)CC1]

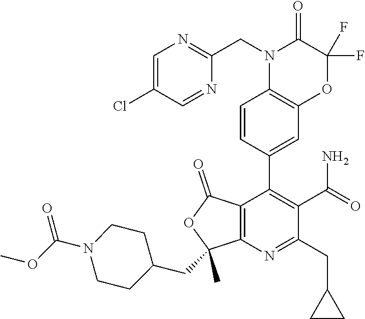 [SMILES: COC(=O)N1CCC(C[C@]2(C)OC(=O)c3c2nc(CC2CC2)c(C(N)=O)c3-c2ccc3c(c2)OC(F)(F)C(=O)N3Cc2ncc(Cl)cn2)CC1]

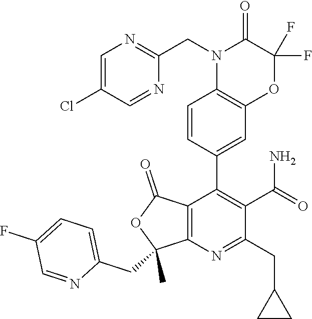 [SMILES: C[C@@]1(Cc2ccc(F)cn2)OC(=O)c2c1nc(CC1CC1)c(C(N)=O)c2-c1ccc2c(c1)OC(F)(F)C(=O)N2Cc1ncc(Cl)cn1]

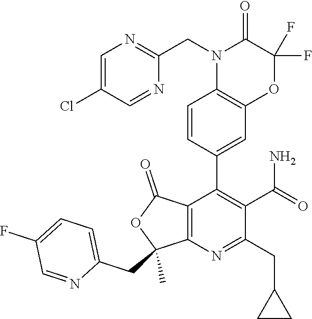 [SMILES: C[C@]1(Cc2ccc(F)cn2)OC(=O)c2c1nc(CC1CC1)c(C(N)=O)c2-c1ccc2c(c1)OC(F)(F)C(=O)N2Cc1ncc(Cl)cn1]

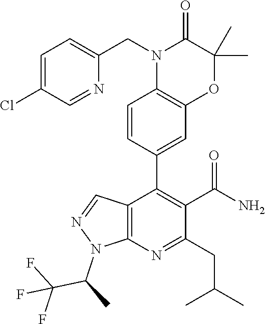

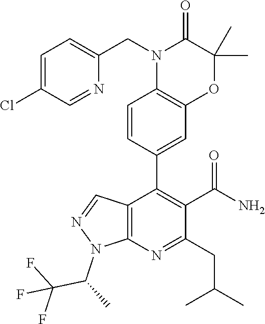

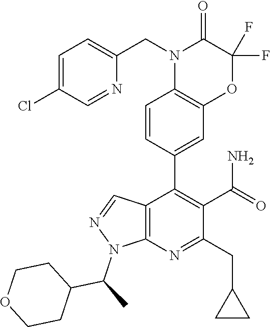 [SMILES: C[C@@H](C1CCOCC1)n1ncc2c(-c3ccc4c(c3)OC(F)(F)C(=O)N4Cc3ccc(Cl)cn3)c(C(N)=O)c(CC3CC3)nc21]

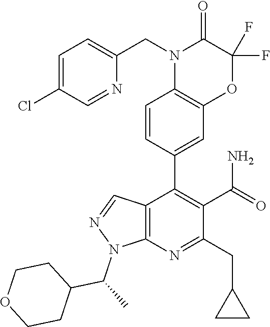 [SMILES: C[C@H](C1CCOCC1)n1ncc2c(-c3ccc4c(c3)OC(F)(F)C(=O)N4Cc3ccc(Cl)cn3)c(C(N)=O)c(CC3CC3)nc21]

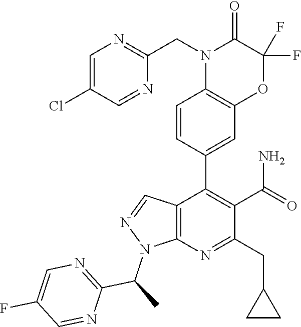 [SMILES: C[C@@H](c1ncc(F)cn1)n1ncc2c(-c3ccc4c(c3)OC(F)(F)C(=O)N4Cc3ncc(Cl)cn3)c(C(N)=O)c(CC3CC3)nc21]

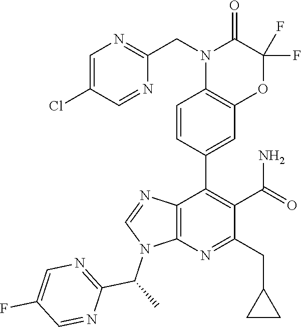 [SMILES: C[C@H](c1ncc(F)cn1)n1cnc2c(-c3ccc4c(c3)OC(F)(F)C(=O)N4Cc3ncc(Cl)cn3)c(C(N)=O)c(CC3CC3)nc21]

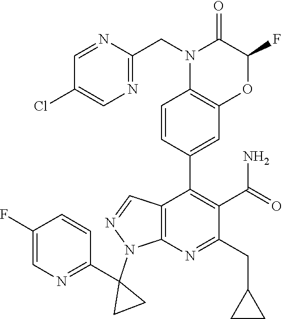 [SMILES: NC(=O)c1c(CC2CC2)nc2c(cnn2C2(c3ccc(F)cn3)CC2)c1-c1ccc2c(c1)O[C@H](F)C(=O)N2Cc1ncc(Cl)cn1]

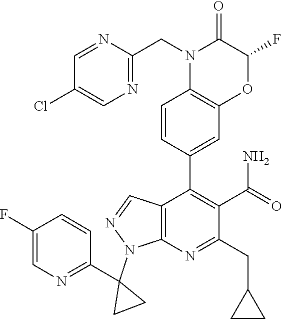 [SMILES: NC(=O)c1c(CC2CC2)nc2c(cnn2C2(c3ccc(F)cn3)CC2)c1-c1ccc2c(c1)O[C@@H](F)C(=O)N2Cc1ncc(Cl)cn1]

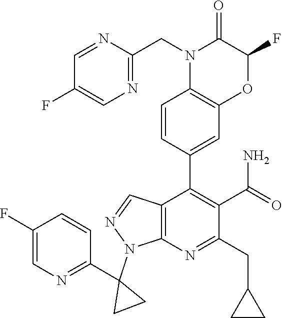 [SMILES: NC(=O)c1c(CC2CC2)nc2c(cnn2C2(c3ccc(F)cn3)CC2)c1-c1ccc2c(c1)O[C@H](F)C(=O)N2Cc1ncc(F)cn1]

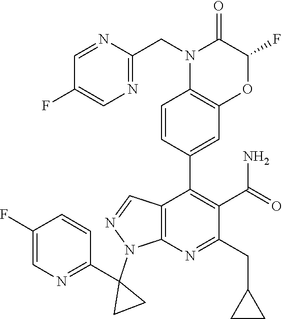 [SMILES: NC(=O)c1c(CC2CC2)nc2c(cnn2C2(c3ccc(F)cn3)CC2)c1-c1ccc2c(c1)O[C@@H](F)C(=O)N2Cc1ncc(F)cn1]

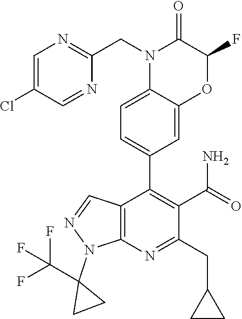 [SMILES: NC(=O)c1c(CC2CC2)nc2c(cnn2C2(C(F)(F)F)CC2)c1-c1ccc2c(c1)O[C@H](F)C(=O)N2Cc1ncc(Cl)cn1]

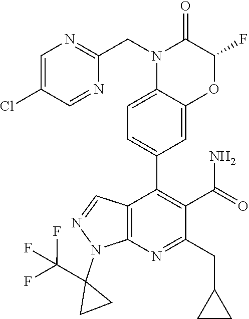 [SMILES: NC(=O)c1c(CC2CC2)nc2c(cnn2C2(C(F)(F)F)CC2)c1-c1ccc2c(c1)O[C@@H](F)C(=O)N2Cc1ncc(Cl)cn1]

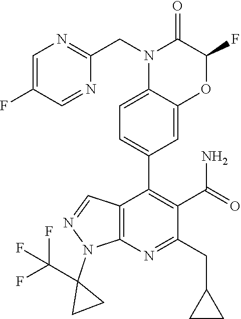 [SMILES: NC(=O)c1c(CC2CC2)nc2c(cnn2C2(C(F)(F)F)CC2)c1-c1ccc2c(c1)O[C@H](F)C(=O)N2Cc1ncc(F)cn1]

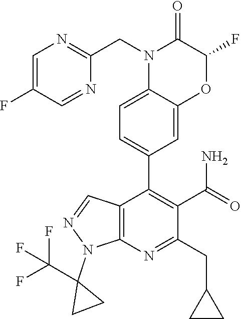 [SMILES: NC(=O)c1c(CC2CC2)nc2c(cnn2C2(C(F)(F)F)CC2)c1-c1ccc2c(c1)O[C@@H](F)C(=O)N2Cc1ncc(F)cn1]

The compounds of Formula I, II, III, IV, V, VI, VII, and VIII, and subformulas thereof include pharmaceutically acceptable salts thereof. In addition, the compounds of Formula I, II, III, IV, V, VI, VII, and VIII, and subformulas thereof also include other salts of such compounds which are not necessarily pharmaceutically acceptable salts, and which may be useful as intermediates for preparing and/or purifying compounds of Formula I, II, III, IV, V, VI, VII, and VIII, subformulas thereof and/or for separating enantiomers of compounds of Formula I, II, III, IV, V, VI, VII, and VIII, and subformulas thereof.

It will further be appreciated that the compounds of Formula I, II, III, IV, V, VI, VII, and VIII, and subformulas or their salts may be isolated in the form of solvates, and accordingly that any such solvate is included within the scope of the present disclosure. For example, compounds of Formula I, II, III, IV, V, VI, VII, and VIII, and subformulas thereof and salts of each thereof can exist in unsolvated as well as solvated forms with pharmaceutically acceptable solvents such as water, ethanol, and the like.

### Pharmaceutical Compositions and Administration

When employed as pharmaceuticals, compounds as described herein (e.g., one or more compounds as disclosed herein, or a stereoisomer or mixture of stereoisomers thereof) can be administered in the form of a pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, one or more compounds as disclosed herein, or a stereoisomer or mixture of stereoisomers thereof, in combination with one or more pharmaceutically acceptable excipients (carriers). For example, a pharmaceutical composition prepared using one or more compounds as disclosed herein, or a stereoisomer or mixture of stereoisomers thereof.

In one embodiment, provided is a pharmaceutical composition comprising a compound, or a stereoisomer or mixture of stereoisomers thereof, or pharmaceutically acceptable salt thereof, as disclosed herein, and a pharmaceutically acceptable excipient. In one embodiment, provided is a pharmaceutical composition comprising a compound, or a stereoisomer or mixture of stereoisomers thereof, or pharmaceutically acceptable salt thereof, as disclosed herein, and a pharmaceutically acceptable excipient, wherein a compound, or a stereoisomer or mixture of stereoisomers thereof, or pharmaceutically acceptable salt thereof, is present in the pharmaceutical composition in an amount greater than about 0.1%, greater than about 1%, greater than about 5%, greater than about 10%, greater than about 15%, greater than about 20%, greater than about 25%, greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than about 95% purity, or about 40%, or about 45%, or about 50%, or about 55%, or about 60%, or about 65%, or about 70%, or about 75%, or about 80%, or about 85%, or about 90%, or about 95%, by weight.

In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing one or more compounds as disclosed herein, or a stereoisomer or mixture of stereoisomers thereof with a pharmaceutically acceptable excipient. Pharmaceutical compositions containing one or more compounds as disclosed herein, or a stereoisomer or mixture of stereoisomers thereof as the active ingredient can be prepared by intimately mixing one or more compounds as disclosed herein, or a stereoisomer or mixture of stereoisomers thereof with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In some embodiments, the compound or pharmaceutical composition can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22nd Edition (Pharmaceutical Press, London, UK. 2012).

In some embodiments, the compounds and pharmaceutical compositions described herein or a pharmaceutical composition thereof can be administered to patient in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal (e.g., intranasal), nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In some embodiments, a route of administration is parenteral (e.g., intratumoral).

In some embodiments, one or more compounds as disclosed herein, or a stereoisomer or mixture of stereoisomers thereof as described herein or pharmaceutical compositions thereof can be formulated for parenteral administration, e.g., formulated for injection via the intraarterial, intrasternal, intracranial, intravenous, intramuscular, sub-cutaneous, or intraperitoneal routes. For example, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure. In some embodiments, devices are used for parenteral administration. For example, such devices may include needle injectors, microneedle injectors, needle-free injectors, and infusion techniques.

In some embodiments, the pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In some embodiments, the form must be sterile and must be fluid to the extent that it may be easily injected. In some embodiments, the form should be stable under the conditions of manufacture and storage and must be preserved against the contaminating action of microorganisms, such as bacteria and fungi.

In some embodiments, the carrier also can be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity can be maintained, for example, by the use of a coating, such as lecithin, by the maintenance of the required particle size in the case of dispersion, and by the use of surfactants. In some embodiments, the prevention of the action of microorganisms can be brought about by various antibacterial and antifungal agents, for example, parabens, chlorobutanol, phenol, sorbic acid, thimerosal, and the like. In some embodiments, isotonic agents, for example, sugars or sodium chloride are included. In some embodiments, prolonged absorption of the injectable compositions can be brought about by the use in the compositions of agents delaying absorption, for example, aluminum monostearate and gelatin.

In some embodiments, sterile injectable solutions are prepared by incorporating one or more compounds as disclosed herein, or a stereoisomer or mixture of stereoisomers thereof in the required amount in the appropriate solvent with various of the other ingredients enumerated above, as required, followed by filtered sterilization. In some embodiments, dispersions are prepared by incorporating the various sterilized active ingredients into a sterile vehicle which contains the basic dispersion medium and the required other ingredients from those enumerated above. In some embodiments, sterile powders are used for the preparation of sterile injectable solutions. In some embodiments, the methods of preparation are vacuum-drying and freeze-drying techniques, which yield a powder of the active ingredient, plus any additional desired ingredient from a previously sterile-filtered solution thereof.

In some embodiments, pharmacologically acceptable excipients usable in a rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol, Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In some embodiments, suppositories can be prepared by mixing one or more compounds as disclosed herein, or a stereoisomer or mixture of stereoisomers thereof, or pharmaceutical compositions as described herein with suitable non-irritating excipients or carriers such as cocoa butter, polyethylene glycol or a suppository wax which are solid at ambient temperature but liquid at body temperature and therefore melt in the rectum and release the active compound. In some embodiments, compositions for rectal administration are in the form of an enema.

In some embodiments, one or more compounds as disclosed herein, or a stereoisomer or mixture of stereoisomers thereof, as described herein or a pharmaceutical composition thereof is formulated for local delivery to the digestive or GI tract by way of oral administration (e.g., solid or liquid dosage forms).

In some embodiments, solid dosage forms for oral administration include capsules, tablets, pills, powders, and granules. In some embodiments, one or more compounds as disclosed herein, or a stereoisomer or mixture of stereoisomers thereof, is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For example, in the case of capsules, tablets and pills, the dosage form may also comprise buffering agents. In some embodiments, solid compositions of a similar type may also be employed as fillers in soft and hard-filled gelatin capsules using such excipients as lactose or milk sugar as well as high molecular weight polyethylene glycols and the like.

In some embodiments, the pharmaceutical compositions will take the form of a unit dosage form such as a pill or tablet and thus the composition may contain, along with one or more compounds as disclosed herein, or a stereoisomer or mixture of stereoisomers thereof as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In some embodiments, another solid dosage form, a powder, marume, solution or suspension (e.g., in propylene carbonate, vegetable oils, PEG's, poloxamer 124 or triglycerides) is encapsulated in a capsule (gelatin or cellulose base capsule). In some embodiments, unit dosage forms in which one or more compounds and pharmaceutical compositions as provided herein or additional active agents are physically separated are also contemplated; e.g., capsules with granules (or tablets in a capsule) of each drug; two-layer tablets; two-compartment gel caps, etc. In some embodiments, enteric coated or delayed release oral dosage forms are also contemplated.

In some embodiments, other physiologically acceptable compounds may include wetting agents, emulsifying agents, dispersing agents or preservatives that are particularly useful for preventing the growth or action of microorganisms. For example, various preservatives are well known and include, for example, phenol and ascorbic acid.

In some embodiments, the excipients are sterile and generally free of undesirable matter. For example, these compositions can be sterilized by conventional, well-known sterilization techniques. In some embodiments, for various oral dosage form excipients such as tablets and capsules, sterility is not required. For example, the United States Pharmacopeia/National Formulary (USP/NF) standard can be sufficient.

In some embodiments, one or more compounds as disclosed herein, or a stereoisomer or mixture of stereoisomers thereof as described herein or a pharmaceutical composition thereof is formulated for ocular administration. In some embodiments, ocular compositions can include, without limitation, one or more of any of the following: viscogens (e.g., carboxymethylcellulose, glycerin, polyvinylpyrrolidone, polyethylene glycol); stabilizers (e.g., Pluronic (triblock copolymers), cyclodextrins); preservatives (e.g., benzalkonium chloride, EDTA, SofZia (boric acid, propylene glycol, sorbitol, and zinc chloride; Alcon Laboratories, Inc.), Purite (stabilized oxychloro complex; Allergan, Inc.)).

In some embodiments, one or more compounds as disclosed herein, or a stereoisomer or mixture of stereoisomers thereof as described herein or a pharmaceutical composition thereof is formulated for topical administration to the skin or mucosa (e.g., dermally or transdermally). In some embodiments, topical compositions can include ointments and creams. In some embodiments, ointments are semisolid preparations that are typically based on petrolatum or other petroleum derivatives. In some embodiments, creams containing the selected active agent are typically viscous liquid or semisolid emulsions, often either oil-in-water or water-in-oil. For example, cream bases are typically water-washable, and contain an oil phase, an emulsifier and an aqueous phase. For example, the oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. In some embodiments, the emulsifier in a cream formulation is generally a nonionic, anionic, cationic or amphoteric surfactant. In some embodiments, as with other carriers or vehicles, an ointment base should be inert, stable, nonirritating and non-sensitizing.

In any of the foregoing embodiments, pharmaceutical compositions as described herein can include one or more one or more of the following: lipids, interbilayer crosslinked multilamellar vesicles, biodegradable poly(D,L-lactic-co-glycolic acid) (PLGA)-based or poly anhydride-based nanoparticles or microparticles, and nanoporous particle-supported lipid bilayers.

The amount of the compound in a pharmaceutical composition or formulation can vary within the full range employed by those skilled in the art. Typically, the formulation will contain, on a weight percent (wt %) basis, from about 0.01-99.99 wt % of a compound of this disclosure based on the total formulation, with the balance being one or more suitable pharmaceutical excipients. In one embodiment, the compound is present at a level of about 1-80 wt %. Representative pharmaceutical formulations are described below.

### Formulation Example 1—Tablet Formulation

The following ingredients are mixed intimately and pressed into single scored tablets.

| Ingredient | Quantity per tablet, mg |
|---|---|
| compound of this disclosure | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule

| Ingredient | Quantity per capsule, mg |
|---|---|
| compound of this disclosure | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| compound of this disclosure | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| coloring | 0.5 | mg |

| | |
|---|---|
| distilled water | q.s. to 100 mL |

### Formulation Example 4—Injectable Formulation

The following ingredients are mixed to form an injectable formulation.

| Ingredient | Amount |
|---|---|
| compound of this disclosure | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of this disclosure with Witepsol® H-15 (triglycerides of saturated vegetable fatty acid; Riches-Nelson, Inc., New York), and has the following composition:

| Ingredient | Amount |
|---|---|
| compound of this disclosure | 500 mg |
| Witepsol ® H-15 | balance |

In some embodiments, the dosage for one or more compounds as disclosed herein, or a stereoisomer or mixture of stereoisomers thereof, is determined based on a multiple factors including, but not limited to, type, age, weight, sex, medical condition of the patient, severity of the medical condition of the patient, route of administration, and activity of the compound or pharmaceutically acceptable s salt, stereoisomer, mixture of stereoisomers, or solvate thereof. In some embodiments, proper dosage for a particular situation can be determined by one skilled in the medical arts. In some embodiments, the total daily dosage may be divided and administered in portions throughout the day or by means providing continuous delivery.

In some embodiments, one or more compounds as disclosed herein, or a stereoisomer or mixture of stereoisomers thereof, is administered at a dose from about 0.01 to about 1000 mg. For example, from about 0.1 to about 30 mg, about 10 to about 80 mg, about 0.5 to about 15 mg, about 50 mg to about 200 mg, about 100 mg to about 300 mg, about 200 to about 400 mg, about 300 mg to about 500 mg, about 400 mg to about 600 mg, about 500 mg to about 800 mg, about 600 mg to about 900 mg, or about 700 mg to about 1000 mg. In some embodiments, the dose is a therapeutically effective amount.

In some embodiments, one or more compounds as disclosed herein, or a stereoisomer or mixture of stereoisomers thereof as described herein is administered at a dosage of from about 0.0002 mg/Kg to about 100 mg/Kg (e.g., from about 0.0002 mg/Kg to about 50 mg/Kg; from about 0.0002 mg/Kg to about 25 mg/Kg; from about 0.0002 mg/Kg to about 10 mg/Kg; from about 0.0002 mg/Kg to about 5 mg/Kg; from about 0.0002 mg/Kg to about 1 mg/Kg; from about 0.0002 mg/Kg to about 0.5 mg/Kg; from about 0.0002 mg/Kg to about 0.1 mg/Kg; from about 0.001 mg/Kg to about 50 mg/Kg; from about 0.001 mg/Kg to about 25 mg/Kg; from about 0.001 mg/Kg to about 10 mg/Kg; from about 0.001 mg/Kg to about 5 mg/Kg; from about 0.001 mg/Kg to about 1 mg/Kg; from about 0.001 mg/Kg to about 0.5 mg/Kg; from about 0.001 mg/Kg to about 0.1 mg/Kg; from about 0.01 mg/Kg to about 50 mg/Kg; from about 0.01 mg/Kg to about 25 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 50 mg/Kg; from about 0.1 mg/Kg to about 25 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg). In some embodiments, one or more compounds as disclosed herein, or a stereoisomer or mixture of stereoisomers thereof as described herein is administered as a dosage of about 100 mg/Kg.

In some embodiments, the foregoing dosages of one or more compounds as disclosed herein, or a stereoisomer or mixture of stereoisomers thereof, can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of one or more compounds as disclosed herein, or a stereoisomer or mixture of stereoisomers thereof as described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, one or more compounds as disclosed herein, or a stereoisomer or mixture of stereoisomers thereof is administered to a patient for a period of time followed by a separate period of time where administration of one or more compounds as disclosed herein, or a stereoisomer or mixture of stereoisomers thereof is stopped. In some embodiments, one or more compounds as disclosed herein, or a stereoisomer or mixture of stereoisomers thereof is administered for a first period and a second period following the first period, with administration stopped during the second period, followed by a third period where administration of one or more compounds as disclosed herein, or a stereoisomer or mixture of stereoisomers thereof is started and then a fourth period following the third period where administration is stopped. For example, the period of administration of one or more compounds as disclosed herein, or a stereoisomer or mixture of stereoisomers thereof followed by a period where administration is stopped is repeated for a determined or undetermined period of time. In some embodiments, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, one or more compounds as disclosed herein, or a stereoisomer or mixture of stereoisomers thereof, is orally administered to the patient one or more times per day (e.g., one time per day, two times per day, three times per day, four times per day per day or a single daily dose).

In some embodiments, one or more compounds as disclosed herein, or a stereoisomer or mixture of stereoisomers thereof, is administered by parenteral administration to the patient one or more times per day (e.g., 1 to 4 times, one time per day, two times per day, three times per day, four times per day or a single daily dose).

In some embodiments, one or more compounds as disclosed herein, or a stereoisomer or mixture of stereoisomers thereof, is administered by parenteral administration to the patient weekly.

### Methods of Treatment

In some embodiments, this disclosure provides methods for treating a subject (e.g., a human) having a disease, disorder, or condition in which inhibition of one or more calcitonin receptor and/or amylin receptor is beneficial for the treatment of the underlying pathology and/or symptoms and/or progression of the disease, disorder, or condition. In some embodiments, the methods provided herein can include treating one or more conditions associated, co-morbid or sequela with any one or more of the conditions provided herein.

Provided herein is a method for treating a calcitonin receptor and/or an amylin receptor associated disease or disorder, the method comprising administering to a subject in need thereof an effective amount of a compound disclosed herein (e.g., a compound of Formula I, II, III, IV, or V, or any subformula thereof or a pharmaceutically acceptable salt, stereoisomer, mixture of stereoisomers, or solvate thereof), or a pharmaceutical composition as disclosed herein. Also provided herein are methods for treating or preventing a calcitonin receptor and/or an amylin receptor associated disease or disorder in a subject in need thereof, the method comprising administering to a subject in need thereof a therapeutically effective amount of a compound disclosed herein (e.g., a compound of Formula I, II, III, IV, or V, or any subformula thereof, or a pharmaceutically acceptable salt, stereoisomer, mixture of stereoisomers, or solvate thereof), or a pharmaceutical composition thereof.

In some embodiments, the calcitonin receptor and/or amylin receptor associated disease or disorder is a bone disorder, a metabolic disorder, pain, a neurodegenerative disease or disorder, a cardiovascular disease, or other disease or disorder as described herein.

In some embodiments, the disease or disorder includes, but is not limited to type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, and Polycystic Ovary Syndrome (PCOS).

In some embodiments, the disease or disorder includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof.

In some embodiments, the disease or disorder includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

In some embodiments, the compounds and pharmaceutical compositions and methods for treating a patient described herein induce one or more of blood glucose reduction (e.g., reduce blood glucose levels), reduce blood hemoglobin A1c (HbA1c) levels, promote insulin synthesis, stimulate insulin secretion, increase the mass of β-cells, modulate gastric acid secretion, modulate gastric emptying, decrease the body mass index (BMI), and/or decrease glucagon production (e.g., level). In certain embodiments, the compounds and pharmaceutical compositions and methods for treating a patient described herein stabilize serum glucose and serum insulin levels (e.g., serum glucose and serum insulin concentrations). Also provided herein are methods for modulating glucose or insulin levels in a patient in need of such modulating, the method comprising administering to the patient an effective amount of one or more compounds as disclosed herein, or a stereoisomer or mixture of stereoisomers thereof, or a pharmaceutical composition as disclosed herein.

In some embodiments, provided herein is a method for reducing the risk (e.g., by about at least 20%, at least 30%, at least 40%, at least 50%, at least 60%, at least 70%, or at least 80%) of major adverse cardiovascular events (MACE) in a patient in need thereof, the method comprising administering to the patient an effective amount of one or more compounds as disclosed herein, or a stereoisomer or mixture of stereoisomers thereof, or a pharmaceutical composition as disclosed herein. In certain of these embodiments, the patient is an adult that has been diagnosed with type 2 diabetes (T2D). In certain embodiments, the patient is an adult that has been diagnosed with a heart disease. In certain embodiments, the patient is an adult that has been diagnosed with type 2 diabetes (T2D) and a heart disease. In certain embodiments, the patient is an adult that has type 2 diabetes (T2D). In certain embodiments, the patient is an adult that has a heart disease. In certain embodiments, the patient has type 2 diabetes (T2D) and a heart disease.

In some embodiments, the calcitonin receptor and/or amylin receptor associated disease or disorder is a bone disorder, including, but not limited to, osteoporosis, Paget's disease, hypercalcemia, Sudeck's atrophy, polystatic fibrous dysplasia, intersemocostoclavicular ossification, osteogenesis imperfecta, osteopenia, periodontal disease or defect, osteolytic bone disease, metastatic bone disorder, or bone loss resulting from a malignancy, autoimmune arthritides, a breakage or fracture, or immobility or disuse.

In some embodiments, the calcitonin receptor and/or amylin receptor associated disease or disorder is pain, including, but not limited to, osteopathic pain, phantom limb pain, general pain, hyperalgesia, or pain associated with diabetic neuropathy.

In some embodiments, the calcitonin receptor and/or amylin receptor associated disease or disorder is a neurodegenerative disease or disorder, including, but not limited to, Alzheimer's disease.

In some embodiments, the calcitonin receptor and/or amylin receptor associated disease or disorder is a metabolic disorder, including, but not limited to, non-alcoholic fatty liver disease (NAFLD), non-alcoholic steatohepatitis (NASH), insulin dependent diabetes, non-insulin dependent diabetes, impaired glucose tolerance, obesity, syndrome X, or other diabetic complication.

In some embodiments, the calcitonin receptor and/or amylin receptor associated disease or disorder is include primary or secondary hyperthyroidism, endocrine disorder, conditions associated with inhibiting gastric secretion, gastrointestinal disorders, renal osteodystrophy, or male infertility.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to alleviate insulin suppression in pancreatic tissue.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to treat alleviate insulin resistance.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to treat impaired glucose tolerance.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to treat obesity and symptoms thereof.

In some embodiments, provided is a method for reducing body fat or body fat gain, comprising administering to a subject in need thereof a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method of altering a body composition of a subject in need of treatment, wherein body fat is reduced and lean body mass is maintained or increased, comprising administering to the subject a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method for reducing body weight in a subject in need of, comprising administering to the subject a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method for reducing caloric intake in a subject in need of reduction thereof, comprising administering to the subject a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method for reducing body fat or body fat gain in a subject in need of treatment while maintaining or increasing loan body mass, comprising administering to the subject a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to treat hypertension.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to treat essential hypertension.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to treat a subject suffering from hypertension and hyperamylinemia.

In some embodiments, provided is a method for treating hyperinsulinemia, comprising administering to a subject in need thereof a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method for treating a hypertensive, insulin-resistant subject suffering from coronary artery disease and having hyperamylinemia or hyperinsulinemia, comprising administering to a subject in need thereof a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method for decreasing basal and submaximally stimulated rates of glycogen synthesis in a subject, comprising administering to a subject in need thereof a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method for decreasing the rate of incorporation of glucose into glycogen in muscle tissue of a subject, comprising administering to a subject in need thereof a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method for treating obesity and hypertension, and the lipid disorders and atherosclerosis associated therewith, in a subject, comprising administering to a subject in need thereof a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to modulate renin activity in a subject in need thereof.

In some embodiments, provided is a method for treating or preventing the development of cardiac failure, in a subject, comprising administering to a subject in need thereof a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to beneficially regulate gastrointestinal motility in a subject in need thereof. In some embodiments, the beneficial regulation of gastrointestinal motility comprises delaying gastric emptying.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to treat postprandial hyperglycemia in a subject in need thereof.

### Obesity

In some embodiments, the condition, disease or disorder is obesity and conditions, diseases or disorders that are associated with or related to obesity. Non-limiting examples of obesity and obesity related conditions include symptomatic obesity, simple obesity, childhood obesity, morbid obesity, and abdominal obesity (central obesity characterized by abdominal adiposity). Non-limiting examples of symptomatic obesity include endocrine obesity (e.g., Cushing syndrome, hypothyroidism, insulinoma, obese type II diabetes, pseudohypoparathyroidism, hypogonadism), hypothalamic obesity, hereditary obesity (e.g., Prader-Willi syndrome, Laurence-Moon-Biedl syndrome), and drug-induced obesity (e.g., steroid, phenothiazine, insulin, sulfonylurea agent, or β-blocker-induced obesity).

In some embodiments, the condition, disease or disorder is associated with obesity. Examples of such conditions, diseases or disorders include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, and metabolic syndrome. In some embodiments, the chemical compound and pharmaceutical compositions described herein can be used to treat patients exhibiting symptoms of both obesity and insulin deficiency.

### Diabetes

In some embodiments, the condition, disease or disorder is diabetes. Non-limiting examples of diabetes include type 1 diabetes mellitus, type 2 diabetes mellitus (e.g., diet-treated type 2-diabetes, sulfonylurea-treated type 2-diabetes, a far-advanced stage type 2-diabetes, long-term insulin-treated type 2-diabetes), diabetes mellitus (e.g., non-insulin-dependent diabetes mellitus, insulin-dependent diabetes mellitus), gestational diabetes, obese diabetes, autoimmune diabetes, and borderline type diabetes. In some embodiments, the condition, disease or disorder is type 2 diabetes mellitus (e.g., diet-treated type 2-diabetes, sulfonylurea-treated type 2-diabetes, a far-advanced stage type 2-diabetes, long-term insulin-treated type 2-diabetes).

Provided herein is a method of treating a diabetes mellitus in a patient, the method comprising (a) determining that the patient has type 2 diabetes mellitus, and (b) administering to the patient a therapeutically effective amount of one or more compounds as disclosed herein, or a stereoisomer or mixture of stereoisomers thereof, or a pharmaceutical composition as disclosed herein.

Provided herein is a method for treating type 2 diabetes mellitus in a patient, the method comprising administering to a patient identified or diagnosed as having type 2 diabetes mellitus a therapeutically effective amount of one or more compounds as disclosed herein, or a stereoisomer or mixture of stereoisomers thereof, or a pharmaceutical composition as disclosed herein.

Also provided herein is a method of treating type 2 diabetes mellitus in a patient in need thereof, the method comprising administering to the patient a therapeutically effective amount of one or more compounds as disclosed herein, or a stereoisomer or mixture of stereoisomers thereof, or a pharmaceutical composition as disclosed herein.

In some embodiments, the compounds and pharmaceutical compositions and methods for treating a patient with a condition, disease, or disorder (e.g., type 2 diabetes mellitus) described herein reduce fasting plasma glucose levels. In some embodiments, the compounds and pharmaceutical compositions and methods for treating a patient with a condition, disease, or disorder (e.g., type 2 diabetes mellitus) described herein reduce non-fasting plasma glucose levels. In some embodiments, the compounds and pharmaceutical compositions and methods for treating a patient with a condition, disease, or disorder (e.g., type 2 diabetes mellitus) described herein reduce HbA1c levels. In some embodiments, the compounds and pharmaceutical compositions and methods for treating a patient with a condition, disease, or disorder (e.g., type 2 diabetes mellitus) described herein reduce glucagon levels. In some embodiments, the compounds and pharmaceutical compositions and methods for treating a patient with a condition, disease, or disorder (e.g., type 2 diabetes mellitus) described herein increase insulin levels. In some embodiments, the compounds and pharmaceutical compositions and methods for treating a patient with a condition, disease, or disorder (e.g., type 2 diabetes mellitus) described herein reduce BMI.

In some embodiments, a reduction in fasting plasma glucose levels of about 5% to about 95% indicates treatment of type 2 diabetes mellitus. In some embodiments, a reduction in fasting plasma glucose levels of about 15% to about 80% indicates treatment of type 2 diabetes mellitus. In some embodiments, a reduction in fasting plasma glucose levels of about 25% to about 60% indicates treatment of type 2 diabetes mellitus. In some embodiments, a reduction in fasting plasma glucose levels to about or below 126 mg/dL, about or below 110 mg/dL, or about or below 90 mg/dL indicates treatment of the type 2 diabetes mellitus.

In some embodiments, a reduction in non-fasting plasma glucose levels of about 5% to about 95% indicates treatment of type 2 diabetes mellitus. In some embodiments, a reduction in non-fasting plasma glucose levels of about 15% to about 80% indicates treatment of type 2 diabetes mellitus. In some embodiments, a reduction in non-fasting plasma glucose levels of about 25% to about 60% indicates treatment of type 2 diabetes mellitus. In some embodiments, a reduction in non-fasting plasma glucose levels to about or below 200 mg/dL, about or below 150 mg/dL, or about or below 130 mg/dL indicates treatment of type 2 diabetes mellitus.

In some embodiments, a reduction in HbA1c levels of about 5% to about 95% indicates treatment of type 2 diabetes mellitus. In some embodiments, a reduction in HbA1c levels of about 15% to about 80% indicates treatment of type 2 diabetes mellitus. In some embodiments, a reduction in HbA1c levels of about 25% to about 60% indicates treatment of type 2 diabetes mellitus. In some embodiments, reduction in HbA1c levels to about or below 6.5%, about or below 6.0%, or about or below 5.0% indicates treatment of type 2 diabetes mellitus.

In some embodiments, a reduction in glucagon levels of about 5% to about 95% indicates treatment of type 2 diabetes mellitus. In some embodiments, a reduction in glucagon levels of about 15% to about 80% indicates treatment of type 2 diabetes mellitus. In some embodiments, a reduction in glucagon levels of about 25% to about 60% indicates treatment of type 2 diabetes mellitus. In some embodiments, an increase in insulin levels of about 5% to about 95% indicates treatment of type 2 diabetes mellitus. In some embodiments, an increase in insulin levels of about 15% to about 80% indicates treatment of type 2 diabetes mellitus. In some embodiments, an increase in insulin levels of about 25% to about 60% indicates treatment of type 2 diabetes mellitus.

In some embodiments, a reduction in BMI of about 5% to about 95% indicates treatment of type 2 diabetes mellitus. In some embodiments, a reduction in BMI of about 15% to about 80% indicates treatment of the type 2 diabetes mellitus. In some embodiments, a reduction in BMI of about 25% to about 60% indicates treatment of type 2 diabetes mellitus. In some embodiments, a reduction in BMI of about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of type 2 diabetes mellitus. In some embodiments, a reduction in BMI to about or below 40, about or below 30, or about or below 20 indicates treatment of type 2 diabetes mellitus.

In some embodiments, the condition, disease or disorder is associated with diabetes (e.g., a complication of diabetes). Non-limiting examples of disorders associated with diabetes include obesity, obesity-related disorders, metabolic syndrome, neuropathy, nephropathy (e.g., diabetic nephropathy), retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, diabetic cachexia, delayed wound healing, diabetic dyslipidemia peripheral blood circulation disorder, cardiovascular risk factors. (e.g., coronary artery disease, peripheral artery disease, cerebrovascular disease, hypertension, and risk factors related to unmanaged cholesterol and/or lipid levels, and/or inflammation), NASH, bone fracture, and cognitive dysfunction

Other non-limiting examples of disorders related to diabetes include pre-diabetes, hyperlipidemia (e.g., hypertriglyceridemia, hypercholesterolemia, high LDL-cholesterolemia, low HDL-cholesterolemia, postprandial hyperlipemia), metabolic syndrome (e.g., metabolic disorder where activation of GLP-1R is beneficial, metabolic syndrome X), hypertension, impaired glucose tolerance (IGT), insulin resistance, and sarcopenia.

In some embodiments, the condition, disease or disorder is diabetes and obesity (diabesity). In some embodiments, the compounds described herein are also useful in improving the therapeutic effectiveness of metformin.

### Disorders of Metabolically Important Tissues

In some embodiments, the condition, disease or disorder is a disorder of a metabolically important tissue. Non-limiting examples of metabolically important tissues include liver, fat, pancreas, kidney, and gut.

In some embodiments, the condition, disease or disorder is a fatty liver disease. Fatty liver diseases include, but are not limited to, non-alcoholic fatty acid liver disease (NAFLD), steatohepatitis, non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, glycogen storage diseases, Weber-Christian disease, Wolman's disease, acute fatty liver of pregnancy, and lipodystrophy.

Non-alcoholic fatty liver disease (NAFLD) represents a spectrum of disease occurring in the absence of alcohol abuse and is typically characterized by the presence of steatosis (fat in the liver). NAFLD is believed to be linked to a variety of conditions, e.g., metabolic syndrome (including obesity, diabetes and hypertriglyceridemia) and insulin resistance. It can cause liver disease in adults and children and may ultimately lead to cirrhosis (Skelly et al., J Hepatol 2001; 35:195-9; Chitturi et al., Hepatology 2002; 35 (2): 373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 Suppl: S186-90). In some embodiments, the patient is a pediatric patient. The term “pediatric patient” as used herein refers to a patient under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age. In some embodiments, the patient is an adult patient.

Other non-limiting examples of disorders in metabolically important tissues include joint disorders (e.g., osteoarthritis, secondary osteoarthritis), steatosis (e.g. in the liver); gall stones; gallbladder disorders; gastroesophageal reflux; sleep apnea; hepatitis; fatty liver; bone disorder characterized by altered bone metabolism, such as osteoporosis, including post-menopausal osteoporosis, poor bone strength, osteopenia, Paget's disease, osteolytic metastasis in cancer patients, osteodistrophy in liver disease and the altered bone metabolism caused by renal failure or hemodialysis, bone fracture, bone surgery, aging, pregnancy, protection against bone fractures, and malnutrition polycystic ovary syndrome; renal disease (e.g., chronic renal failure, glomerulonephritis, glomerulosclerosis, nephrotic syndrome, hypertensive nephrosclerosis, end-stage renal disease); muscular dystrophy, angina pectoris, acute or chronic diarrhea, testicular dysfunction, respiratory dysfunction, frailty, sexual dysfunction (e.g., erectile dysfunction), and geriatric syndrome. In some embodiments, the compounds and pharmaceutical compositions described herein can be used for treating surgical trauma by improving recovery after surgery and/or by preventing the catabolic reaction caused by surgical trauma.

### Cardiovascular and Vascular Diseases

In some embodiments, the disease or disorder is a cardiovascular disease. Non-limiting examples of cardiovascular disease include congestive heart failure, atherosclerosis, arteriosclerosis, coronary heart disease, coronary artery disease, congestive heart failure, coronary heart disease, hypertension, cardiac failure, cerebrovascular disorder (e.g., cerebral infarction), vascular dysfunction, myocardial infarction, elevated blood pressure (e.g., 130/85 mm Hg or higher), and prothrombotic state (exemplified by high fibrinogen or plasminogen activator inhibitor in the blood).

In some embodiments, the disease or disorder is related to a vascular disease. Non-limiting examples of vascular diseases include peripheral vascular disease, macrovascular complications (e.g., stroke), vascular dysfunction, peripheral artery disease, abdominal aortic aneurysm, carotid artery disease, cerebrovascular disorder (e.g., cerebral infarction), pulmonary embolism, chronic venous insufficiency, critical limb ischemia, retinopathy, nephropathy, and neuropathy.

### Neurological Diseases

In some embodiments, the disease or disorder is a neurological disorder (e.g., neurodegenerative disorder) or a psychiatric disorder. Non-limiting examples of neurological disorders include brain insulin resistance, mild cognitive impairment (MCI), Alzheimer's disease (AD), Parkinson's disease (PD), anxiety, dementia (e.g., senile dementia), traumatic brain injury, Huntington's chores, tardive dyskinesia, hyperkinesia, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), and chronic wasting syndrome). See, e.g., US2006/0275288A1.

Non-limiting examples of psychiatric disorders include drug dependence/addiction (narcotics and amphetamines and attention deficit/hyperactivity disorder (ADHD). The compounds and pharmaceutical compositions described herein can be useful in improving behavioral response to addictive drugs, decreasing drug dependence, prevention drug abuse relapse, and relieving anxiety caused by the absence of a given addictive substance. See, e.g., US2012/0021979A1.

In some embodiments, the compounds and pharmaceutical compositions described herein are useful in improving learning and memory by enhancing neuronal plasticity and facilitation of cellular differentiation, and also in preserving dopamine neurons and motor function in Morbus Parkinson.

### Insulin-Related Conditions and Disorders

In some embodiments, the disease or disorder is impaired fasting glucose (IFG), impaired fasting glycemia (IFG), hyperglycemia, insulin resistance (impaired glucose homeostasis), hyperinsulinemia, elevated blood levels of fatty acids or glycerol, a hypoglycemic condition, insulin resistant syndrome, paresthesia caused by hyperinsulinemia, hyperlipidemia, hypercholesteremia, impaired wound healing, leptin resistance, glucose intolerance, increased fasting glucose, dyslipidemia (e.g., hyperlipidemia, atherogenic dyslipidemia characterized by high triglycerides and low HDL cholesterol), glucagonoma, hyperprolactinemia, hypoglycemia (e.g., nighttime hypoglycemia), and concomitant comatose endpoint associated with insulin.

In some embodiments, the compounds and pharmaceutical compositions described herein can reduce or slow down the progression of borderline type, impaired fasting glucose or impaired fasting glycemia into diabetes.

### Autoimmune Disorders

In some embodiments, the disease or disorder is an autoimmune disorder. Non-limiting examples of autoimmune disorders include multiple sclerosis, experimental autoimmune encephalomyelitis, autoimmune disorder is associated with immune rejection, graft versus host disease, uveitis, optic neuropathics, optic neuritis, transverse myelitis, inflammatory bowel disease, rheumatoid arthritis, ankylosing spondylitis, systemic lupus erythematosus, myasthenia gravis, and Graves' disease. See, e.g., US20120148586A1.

### Stomach and Intestine-Related Disorders

In some embodiments, the disease or disorder is a stomach or intestine related disorder. Non-limiting examples of these disorders include ulcers of any etiology (e.g. peptic ulcers, Zollinger-Ellison syndrome, drug-induced ulcers, ulcers related to infections or other pathogens), digestion disorders, malabsorption, short bowel syndrome, cul-de-sac syndrome, inflammatory bowel diseases (Crohn's disease and ulcerative colitis), celiac sprue, hypogammaglobulinemic sprue, chemotherapy and/or radiation therapy-induced mucositis and diarrhea, gastrointestinal inflammation, short bowel syndrome, colitis ulcerosa, gastric mucosal injury (e.g., gastric mucosal injury caused by aspirin), small intestinal mucosal injury, and cachexia (e.g., cancerous cachexia, tuberculous cachexia, cachexia associated with blood disease, cachexia associated with endocrine disease, cachexia associated with infectious disease, and cachexia caused by acquired immunodeficiency syndrome).

### Body Weight

In some embodiments, the compounds and pharmaceutical compositions described herein can be used to reduce body weight (e.g., excess body weight), prevent body weight gain, induce weight loss, decrease body fat, or reduce food intake in a patient (e.g., a patient in need thereof). In some embodiments, the weight increase in a patient may be attributed to excessive ingestion of food or unbalanced diets, or may be weight increase derived from a concomitant drug (e.g., insulin sensitizers having a PPARγ agonist-like action, such as troglitazone, rosiglitazone, englitazone, ciglitazone, pioglitazone and the like). In some embodiments, the weight increase may be weight increase before reaching obesity, or may be weight increase in an obese patient. In some embodiments, the weight increase may also be medication-induced weight gain or weight gain subsequent to cessation of smoking.

In some embodiments, the disease or disorder is an eating disorder, such as hyperphagia, binge eating, bulimia, or compulsive eating.

### Inflammatory Diseases

In some embodiments, the disease or disorder is an inflammatory disorder. Non-limiting examples of inflammatory disorders include chronic rheumatoid arthritis, spondylitis deformans, arthritis deformans, lumbago, gout, post-operational or post-traumatic inflammation, bloating, neuralgia, laryngopharyngitis, cystitis, pneumonia, pancreatitis, enteritis, inflammatory bowel disease (including inflammatory large bowel disease), inflammation in metabolically important tissues including liver, fat, pancreas, kidney and gut, and a proinflammatory state (e.g., elevated levels of proinflammatory cytokines or markers of inflammation-like C-reactive protein in the blood).

### Cancer

In some embodiments, the disease or disorder is cancer. Suitable examples of cancer include breast cancer (e.g., invasive ductal breast cancer, noninvasive ductal breast cancer, inflammatory breast cancer), prostate cancer (e.g., hormone-dependent prostate cancer, hormone-independent prostate cancer), pancreatic cancer (e.g., ductal pancreatic cancer), gastric cancer (e.g., papillary adenocarcinoma, mucous adenocarcinoma, adenosquamous carcinoma), lung cancer (e.g., non-small cell lung cancer, small-cell lung cancer, malignant mesothelioma), colon cancer (e.g., gastrointestinal stromal tumor), rectal cancer (e.g., gastrointestinal stromal tumor), colorectal cancer (e.g., familial colorectal cancer, hereditary non-polyposis colorectal cancer, gastrointestinal stromal tumor), small intestinal cancer (e.g., non-Hodgkin's lymphoma, gastrointestinal stromal tumor), esophageal cancer, duodenal cancer, tongue cancer, pharyngeal cancer (e.g., nasopharyngeal cancer, oropharynx cancer, hypopharyngeal cancer), salivary gland cancer, brain tumor (e.g., pineal astrocytoma, pilocytic astrocytoma, diffuse astrocytoma, anaplastic astrocytoma), neurilemmoma, liver cancer (e.g., primary liver cancer, extrahepatic bile duct cancer), renal cancer (e.g., renal cell cancer, transitional cell cancer of the renal pelvis and ureter), bile duct cancer, endometrial cancer, uterine cervical cancer, ovarian cancer (e.g., epithelial ovarian cancer, extragonadal germ cell tumor, ovarian germ cell tumor, ovarian tumor of low malignant potential), bladder cancer, urethral cancer, skin cancer (e.g., intraocular (ocular) melanoma, Merkel cell carcinoma), hemangioma, malignant lymphoma, malignant melanoma, thyroid cancer (e.g., medullary thyroid cancer), parathyroid cancer, nasal cavity cancer, sinus cancer, bone tumor (e.g., osteosarcoma, Ewing tumor, uterine sarcoma, soft tissue sarcoma), angiofibroma, sarcoma of the retina, penis cancer, testicular tumor, pediatric solid tumor (e.g., Wilms' tumor, childhood kidney tumor), Kaposi's sarcoma, Kaposi's sarcoma caused by AIDS, tumor of maxillary sinus, fibrous histiocytoma, leiomyosarcoma, rhabdomyosarcoma, and leukemia (e.g., acute myeloid leukemia, acute lymphoblastic leukemia).

### Hypothalamic-Pituitary Disorders

In some embodiments, the disease or disorder is related to the hypothalamic-pituitary-gonadal axis. For example, the condition, disease or disorder is related to the hypothalamus-pituitary-ovary axis. In another example, the condition, disease or disorder is related to the hypothalamus-pituitary-testis axis. Hypothalamic-pituitary-gonadal axis diseases include, but are not limited to, hypogonadism, polycystic ovary syndrome, hypothyroidism, hypopituitarism, sexual dysfunction, and Cushing's disease.

In some embodiments, the disease or disorder associated with diabetes is related to the hypothalamic-pituitary-gonadal axis.

### Pulmonary Disease

In some embodiments, the disease or disorder is related to a pulmonary disease. Pulmonary diseases include, but are not limited to, asthma, idiopathic pulmonary fibrosis, pulmonary hypertension, obstructive sleep apnoea-hypopnoea syndrome, and chronic obstructive pulmonary disease (COPD) (e.g., emphysema, chronic bronchitis, and refractory (non-reversible) asthma).

In some embodiments, the disease or disorder associated with diabetes is a pulmonary disease.

### Combination Therapy

In some embodiments, this disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In some embodiments, the methods described herein include administering a compound described herein in combination with one or more of a diet therapy (e.g., dietary monitoring, diet therapy for diabetes), an exercise therapy (e.g., physical activity), blood sugar monitoring, gastric electrical stimulation (e.g., TANTALUS®), and diet modifications.

In some embodiments, one or more compounds as disclosed herein, or a stereoisomer or mixture of stereoisomers thereof can be administered in combination with one or more additional therapeutic agents.

Representative additional therapeutic agents include, but are not limited to, anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, anti-oxidants, therapeutic agents for osteoporosis, vitamins, antidementia drugs, erectile dysfunction drugs, therapeutic drugs for urinary frequency or urinary incontinence, therapeutic agents for NAFLD, therapeutic agents for NASH, therapeutic agents for dysuria and anti-emetic agents.

In some embodiments, the one or more additional therapeutic agents include those useful, for example, as anti-obesity agents. Non-limiting examples include monoamine uptake inhibitors (e.g., tramadol, phentermine, sibutramine, mazindol, fluoxetine, tesofensine), serotonin 2C receptor agonists (e.g., lorcaserin), serotonin 6 receptor antagonists, histamine H3 receptor modulator, GABA modulator (e.g., topiramate), including GABA receptor agonists (e.g., gabapentin, pregabalin), neuropeptide Y antagonists (e.g., velneperit), cannabinoid receptor antagonists (e.g., rimonabant, taranabant), ghrelin antagonists, ghrelin receptor antagonists, ghrelin acylation enzyme inhibitors, opioid receptor antagonists (e.g., GSK-1521498), orexin receptor antagonists, melanocortin 4 receptor agonists, 11β-hydroxysteroid dehydrogenase inhibitors (e.g., AZD-4017, BVT-3498, INCB-13739), pancreatic lipase inhibitors (e.g., orlistat, cetilistat), β3 agonists (e.g., N-5984), diacylglycerol acyltransferase 1 (DGAT1) inhibitors, acetylCoA carboxylase (ACC) inhibitors, stearoyl-CoA desaturated enzyme inhibitors, microsomal triglyceride transfer protein inhibitors (e.g., R-256918), sodium-glucose cotransporter 2 (SGLT-2) inhibitors (e.g., JKJ=28431754, dapagliflozin, AVE2268, TS-033, YM543, TA-7284, ASP1941, remogliflozin), NFK inhibitors (e.g., HE-3286), PPAR agonists (e.g., GFT-505, DRF-11605, gemfibrozil and fenofibrate), phosphotyrosine phosphatase inhibitors (e.g., sodium vanadate, trodusquemin), GPR119 agonists (e.g., PSN-821, MBX-2982, APD597), glucokinase activators (e.g., piragliatin, AZD-1656, AZD6370, TTP-355, compounds described in WO006/112549, WO007/028135, WO008/047821, WO008/050821, WO008/136428 and WO008/156757), leptin, leptin derivatives (e.g., metreleptin), leptin resistance improving drugs, CNTF (ciliary neurotrophic factor), BDNF (brain-derived neurotrophic factor), cholecystokinin agonists, amylin preparations (e.g., pramlintide, AC-2307), neuropeptide Y agonists (e.g., PYY3-36, derivatives of PYY3-36, obineptide, TM-30339, TM-30335), oxyntomodulin (OXM) preparations, appetite suppressants (e.g. ephedrine), FGF21 preparations (e.g., animal FGF21 preparations extracted from the pancreas of bovine or swine; human FGF21 preparations genetically synthesized using Escherichia coli or yeast; fragments or derivatives of FGF21), anorexigenic agents (e.g., P-57), human proislet peptide (HIP), farnesoid X receptor (FXR) agonist, phentermine, zonisamide, norepinephrine/dopamine reuptake inhibitor, GDF-15 analog, methionine aminopeptidase 2 (MetAP2) inhibitor, diethylpropion, phendimetrazine, benzphetamine, fibroblast growth factor receptor (FGFR) modulator, and AMP-activated protein kinase (AMPK) activator.

In some embodiments, one or more compounds as disclosed herein, or a stereoisomer or mixture of stereoisomers thereof can be administered in combination with one or more additional therapeutic agents, wherein the additional therapeutic agent is a GLP-1 agonist or exhibits GLP-1 agonist activity.

In some embodiments, the additional therapeutic agent is TTP273, LY2944876 (pegapamodutide), HDM1002, K-757, K-833, retatrutide, IBI362 (mazdutide), cotadutide, AMG133, CT-868, HRS9531, HS-20094, dapiglutide, efinopegdutide, efocipegtrutide, pemvidutide, survodutide, AP026, AZD9550, BGM0504, CT-388, DD01, DR10624, G3215, GMA106, HEC88473, HZ010, LY3493269, MWN101, NN9487, NN9541, RAY1225, SCO-094, SHR-1816, TB001, VK2735, ZP2929, ecnoglutide, GX-G6, GZR18, HRS-7535, YH14617, avexitide, froniglutide, pegsebrenatide, vurolenatide, JY09, NB1001, Byetalog, GW002, HL08, KN056, SAL0112, SHR2042, VCT220, ZT002, ZYOG1, or utreglutide.

In some embodiments, the additional therapeutic agent is endogenous GLP-1, endogenous glucagon, oxyntomodulin, exendin-4, exenatide, lixisenatide, albiglutide, beinaglutide, dulaglutide, efpeglenatide, langlenatide, liraglutide, semaglutide, taspoglutide, tirzepatide, pegapamodutide, lithium chloride, PF-06882961 (danuglipron), LY3502970 (orforglipron), ECC-5004, GSBR-1290, AZD0186, PF-07081532 (lotiglipron), VCT220, TERN-601, RGT-075, CT-996, MDR-001, SAL0112, XW014, AVE-0010, S4P, or Boc5),

In some embodiments, one or more compounds as disclosed herein, or a stereoisomer or mixture of stereoisomers thereof can be administered in combination with one or more additional therapeutic agents, wherein the additional therapeutic agent is selected from a compound disclosed in WO2021/155841, WO/2018/109607, WO/2018/056453, WO/2019/239319, or WO/2019/239371.

In some embodiments, the one or more additional therapeutic agents include those useful, for example, as anti-diabetic agents. Non-limiting examples include insulin and insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS-1), oral insulin preparation, synthetic human insulin), insulin sensitizers (e.g., pioglitazone or a salt thereof), biguanides (e.g., metformin, buformin or a salt thereof (e.g., hydrochloride, fumarate, succinate)), glucagon analogs (e.g., any of glucagon analogs described, e.g., in WO 2010/011439), agents which antagonize the actions of or reduce secretion of glucagon, sulfonylurea agents (e.g., chlorpropamide, tolazamide, gliclazide, glimepiride, tolbutamide, glibenclamide, gliclazide, acetohexamide, glyclopyramide, glybuzole, glyburide), thiazolidinedione agents (e.g. rosiglitazone or pioglitazone), α-glucosidase inhibitors (e.g., voglibose, acarbose, miglitol, emiglitate), insulin secretagogues, such as prandial glucose regulators (sometimes called “short-acting secretagogues”), e.g., meglitinides (e.g. repaglinide and nateglinide), cholinesterase inhibitors (e.g., donepezil, galantamine, rivastigmine, tacrine), NMDA receptor antagonists, dual GLP-1/GIP receptor agonists (e.g., LBT-2000, ZPD1-70), GLP-1R agonists (e.g., exenatide, liraglutide, albiglutide, dulaglutide, abiglutide, taspoglutide, lixisenatide, semaglutide, AVE-0010, S4P and Boc5), and dipeptidyl peptidase IV (DPP-4) inhibitors (e.g., vildagliptin, dutogliptin, gemigliptin, alogliptin, saxagliptin, sitagliptin, linagliptin, berberine, adogliptin, BI1356, GRC8200, MP-513, PF-00734200, PHX1149, SK-0403, ALS2-0426, TA-6666, TS-021, KRP-104, trelagliptin).

In some embodiments, the one or more additional therapeutic agents include those useful, for example, for treating NAFL and NASH. Non-limiting examples include FXR agonists, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21), a niacin analogue, a leukotriene D4 (LTD4) receptor antagonist, an acetyl-CoA carboxylase (ACC) inhibitor, a ketohexokinase (KHK) inhibitor, an apoptosis signal-regulating kinase 1 (ASK1) inhibitor, an ileal bile acid transporter (IBAT) inhibitor, glycyrrhizin, Schisandra extract, ascorbic acid, glutathione, silymarin, lipoic acid, and d-alpha-tocopherol, ascorbic acid, glutathione, vitamin B-complex, glitazones/thiazolidinediones (e.g., troglitazone, rosiglitazone, pioglitazone), metformin, cysteamine, sulfonylureas, alpha-glucosidase inhibitors, meglitinides, vitamin E, tetrahydrolipstatin, milk thistle protein, anti-virals, and anti-oxidants.

In some embodiments, the one or more additional therapeutic agents include those useful, for example, for treating diabetic complications. Non-limiting examples include aldose reductase inhibitors (e.g., tolrestat, epalrestat, zopolrestat, fidarestat, CT-112, ranirestat, lidorestat), neurotrophic factor and increasing agents thereof (e.g., NGF, NT-3, BDNF, neurotrophic production/secretion promoting agents described in WO01/14372 (e.g., 4-(4-chlorophenyl)-2-(2-methyl-1-imidazolyl)-5-[3-(2-methylphenoxyl)propyl]oxazole), compounds described in WO2004/039365), PKC inhibitors (e.g., ruboxistaurin mesylate), AGE inhibitors (e.g., ALT946, N-phenacylthiazolium bromide (ALT766), EXO-226, pyridorin, pyridoxamine), serotonin and noradrenalin reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), and apoptosis signal regulating kinase-1 (ASK-1) inhibitors.

In some embodiments, the one or more additional therapeutic agents include those useful, for example, for treating hyperlipidemia. Non-limiting examples include HMG-COA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compounds described in WO97/10224, e.g., N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidin-4-acetic acid), fibrate compounds (e.g., bezafibrate, clofibrate, simfibrate, clinofibrate), anion exchange resin (e.g., colestyramine), nicotinic acid drugs (e.g., nicomol, niceritrol, niaspan), phytosterols (e.g., soysterol, gamma oryzanol (7-oryzanol)), cholesterol absorption inhibitors (e.g., zechia), CETP inhibitors (e.g., dalcetrapib, anacetrapib) and ω-3 fatty acid preparations (e.g., ω-3-fatty acid ethyl esters 90).

In some embodiments, the one or more additional therapeutic agents include those useful, for example, as anti-hypertensive agents. Non-limiting examples include angiotensin converting enzyme inhibitors (e.g., captopril, enalapril, delapril), angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil), calcium antagonists (e.g., manidipine, nifedipine, amlodipine, efonidipine, nicardipine, cilnidipine) and β-blockers (e.g., metoprolol, atenolol, propranolol, carvedilol, pindolol).

In some embodiments, the one or more additional therapeutic agents include those useful, for example, as diuretics. Non-limiting examples include xanthine derivatives (e.g., theobromine sodium salicylate, theobromine calcium salicylate), thiazide preparations (e.g., ethiazide, cyclopenthiazide, trichloromethiazide, hydrochlorothiazide, hydroflumethiazide, benzylhydrochlorothiazide, penfluthiazide, polythiazide, methyclothiazide), antialdosterone preparations (e.g., spironolactone, triamterene), carbonic anhydrase inhibitors (e.g., acetazolamide) and chlorobenzenesulfonamide agents (e.g., chlortalidone, mefruside, indapamide).

In some embodiments, the one or more additional therapeutic agents include those useful, for example, as immunotherapeutic agents. Non-limiting examples include microbial or bacterial compounds (e.g., muramyl dipeptide derivative, picibanil), polysaccharides having immunoenhancing activity (e.g., lentinan, sizofiran, krestin), cytokines obtained by genetic engineering approaches (e.g., interferon, interleukin (IL) such as IL-1, IL-2, IL-12), and colony-stimulating factors (e.g., granulocyte colony-stimulating factor, erythropoietin).

In some embodiments, the one or more additional therapeutic agents include those useful, for example, as anti-thrombotic agents. Non-limiting examples include heparins (e.g., heparin sodium, heparin calcium, enoxaparin sodium, dalteparin sodium) warfarin (e.g., warfarin potassium); anti-thrombin drugs (e.g., aragatroban, dabigatran) FXa inhibitors (e.g., rivaroxaban, apixaban, edoxaban, betrixaban, YM150, compounds described in WO02/06234, WO2004/048363, WO2005/030740, WO2005/058823, and WO2005/113504) thrombolytic agents (e.g., urokinase, tisokinase, alteplase, nateplase, monteplase, pamiteplase), and platelet aggregation inhibitors (e.g., ticlopidine hydrochloride, clopidogrel, prasugrel, E5555, SHC530348, cilostazol, ethyl icosapentate, beraprost sodium, and sarpogrelate hydrochloride).

In some embodiments, the one or more additional therapeutic agents include those useful, for example, for treating osteoporosis. Non-limiting examples include alfacalcidol, calcitriol, elcatonin, calcitonin salmon, estriol, ipriflavone, pamidronate disodium, alendronate sodium hydrate, incadronate disodium, and risedronate disodium. Suitable examples of vitamins include vitamin B1 and vitamin B12. Suitable examples of erectile dysfunction drugs include apomorphine and sildenafil citrate. Suitable examples of therapeutic agents for urinary frequency or urinary incontinence include flavorxate hydrochloride, oxybutynin hydrochloride and propiverine hydrochloride. Suitable examples of therapeutic agents for dysuria include acetylcholine esterase inhibitors (e.g., distigmine). Suitable examples of anti-inflammatory agents include nonsteroidal anti-inflammatory drugs such as aspirin, acetaminophen, indomethacin.

Other exemplary additional therapeutic agents include agents that modulate hepatic glucose balance (e.g., fructose 1,6-bisphosphatase inhibitors, glycogen phosphorylase inhibitors, glycogen synthase kinase inhibitors, glucokinase activators), agents designed to treat the complications of prolonged hyperglycemia, such as aldose reductase inhibitors (e.g. epalrestat and ranirestat), agents used to treat complications related to micro-angiopathies, anti-dyslipidemia agents, such as HMG-COA reductase inhibitors (statins, e.g. rosuvastatin), cholesterol-lowering agents, bile acid sequestrants (e.g., cholestyramine), cholesterol absorption inhibitors (e.g. plant sterols such as phytosterols), cholesteryl ester transfer protein (CETP) inhibitors, inhibitors of the ileal bile acid transport system (IBAT inhibitors), bile acid binding resins, nicotinic acid (niacin) and analogues thereof, anti-oxidants (e.g., probucol), omega-3 fatty acids, antihypertensive agents, including adrenergic receptor antagonists, such as beta blockers (e.g. atenolol), alpha blockers (e.g. doxazosin), and mixed alpha/beta blockers (e.g. labetalol), adrenergic receptor agonists, including alpha-2 agonists (e.g. clonidine), angiotensin converting enzyme (ACE) inhibitors (e.g. lisinopril), calcium channel blockers, such as dihydropridines (e.g. nifedipine), phenylalkylamines (e.g. verapamil), and benzothiazepines (e.g. diltiazem), angiotensin II receptor antagonists (e.g. candesartan), aldosterone receptor antagonists (e.g. eplerenone), centrally acting adrenergic drugs, such as central alpha agonists (e.g. clonidine), diuretic agents (e.g. furosemide), haemostasis modulators, including antithrombotics (e.g., activators of fibrinolysis), thrombin antagonists, factor VIIa inhibitors, anticoagulants (e.g., vitamin K antagonists such as warfarin), heparin and low molecular weight analogues thereof, factor Xa inhibitors, and direct thrombin inhibitors (e.g. argatroban), antiplatelet agents (e.g., cyclooxygenase inhibitors (e.g. aspirin)), adenosine diphosphate (ADP) receptor inhibitors (e.g. clopidogrel), phosphodiesterase inhibitors (e.g. cilostazol), glycoprotein IIB/IIA inhibitors (e.g. tirofiban), adenosine reuptake inhibitors (e.g. dipyridamole), noradrenergic agents (e.g. phentermine), serotonergic agents (e.g. sibutramine), diacyl glycerolacyltransferase (DGAT) inhibitors, feeding behavior modifying agents, pyruvate dehydrogenase kinase (PDK) modulators, serotonin receptor modulators, monoamine transmission-modulating agents, such as selective serotonin reuptake inhibitors (SSRI) (e.g. fluoxetine), noradrenaline reuptake inhibitors (NARI), noradrenaline-serotonin reuptake inhibitors (SNRI), and monoamine oxidase inhibitors (MAOI) (e.g. toloxatone and amiflamine), compounds described in WO007/013694, WO2007/018314, WO2008/093639 and WO2008/099794, GPR40 agonists (e.g., fasiglifam or a hydrate thereof, compounds described in WO2004/041266, WO2004/106276, WO2005/063729, WO2005/063725, WO2005/087710, WO2005/095338, WO2007/013689 and WO2008/001931), SGLT1 inhibitors, adiponectin or agonist thereof, IKK inhibitors (e.g., AS-2868), somatostatin receptor agonists, ACC2 inhibitors, cachexia-ameliorating agents, such as a cyclooxygenase inhibitors (e.g., indomethacin), progesterone derivatives (e.g., megestrol acetate), glucocorticoids (e.g., dexamethasone), metoclopramide agents, tetrahydrocannabinol agents, agents for improving fat metabolism (e.g., eicosapentaenoic acid), growth hormones, IGF-1, antibodies against a cachexia-inducing factor TNF-α, LIF, IL-6, and oncostatin M, metabolism-modifying proteins or peptides such as glucokinase (GK), glucokinase regulatory protein (GKRP), uncoupling proteins 2 and 3 (UCP2 and UCP3), peroxisome proliferator-activated receptor a (PPARa), MC4r agonists, insulin receptor agonist, PDE 5 inhibitors, glycation inhibitors (e.g., ALT-711), nerve regeneration-promoting drugs (e.g., Y-128, VX853, prosaptide), antidepressants (e.g., desipramine, amitriptyline, imipramine), antiepileptic drugs (e.g., lamotrigine, trileptal, keppra, zonegran, pregabalin, harkoseride, carbamazepine), antiarrhythmic drugs (e.g., mexiletine), acetylcholine receptor ligands (e.g., ABT-594), endothelin receptor antagonists (e.g., ABT-627), narcotic analgesics (e.g., morphine), α2 receptor agonists (e.g., clonidine), local analgesics (e.g., capsaicin), antianxiety drugs (e.g., benzothiazepine), phosphodiesterase inhibitors (e.g., sildenafil), dopamine receptor agonists (e.g., apomorphine), cytotoxic antibodies (e.g., T-cell receptor and IL-2 receptor-specific antibodies), B cell depleting therapies (e.g., anti-CD20 antibody (e.g., rituxan), i-BLyS antibody), drugs affecting T cell migration (e.g., anti-integrin alpha 4/beta 1 antibody (e.g., tysabri), drugs that act on immunophilins (e.g., cyclosporine, tacrolimus, sirolimus, rapamicin), interferons (e.g., IFN-β), immunomodulators (e.g., glatiramer), TNF-binding proteins (e.g., circulating receptors), immunosupressants (e.g., mycophenolate), and metaglidasen, AMG-131, balaglitazone, MBX-2044, rivoglitazone, aleglitazar, chiglitazar, lobeglitazone, PLX-204, PN-2034, GFT-505, THR-0921, exenatide, exendin-4, memantine, midazolam, ketoconazole, ethyl icosapentate, clonidine, azosemide, isosorbide, ethacrynic acid, piretanide, bumetanide, etoposide, piroxicam, NO donating agents (e.g., organonitrates), and NO promoting agents (e.g., phosphodiesterase inhibitors).

In some embodiments, the one or more additional therapeutic agents include those useful, for example, as anti-emetic agents. As used herein, an “anti-emetic” agent refers to any agent that counteracts (e.g., reduces or removes) nausea or emesis (vomiting). It is to be understood that when referring to a therapeutically effective amount of an anti-emetic agent, the amount administered is an amount needed to counteract (e.g., reduce or remove) nausea or emesis (vomiting). While not wishing to be bound by theory, it is believed that administering one or more anti-emetic agents in combination with the formula (I) compounds described herein may allow higher dosages of the formula (I) compounds to be administered, e.g., because the patient may be able to have a normal food intake and thereby respond faster to the treatment.

Non-limiting examples of anti-emetic agents include 5HT3-receptor antagonists (serotonin receptor antagonists), neuroleptics/anti-psychotics, antihistamines, anticholinergic agents, steroids (e.g., corticosteroids), NK1-receptor antagonists (e.g., Neurokinin 1 substance P receptor antagonists), antidopaminergic agents/dopamine receptor antagonists, benzodiazepines, cannabinoids.

For example, the antiemetic agent can be selected from the group consisting of; neuroleptics, antihistamines, anti-cholinergic agents, steroids, 5HT-3-receptor antagonists, NK1-receptor antagonists, anti-dopaminergic agents/dopamine receptor antagonists, benzodiazepines and non-psychoactive cannabinoids.

In some embodiments, the anti-emetic agent is a 5HT3-receptor antagonist (serotonin receptor antagonist). Non-limiting examples of 5HT3-receptor antagonists (serotonin receptor antagonists) include: granisetron (Kytril), dolasetron, ondansetron (Zofran), tropisetron, ramosetron, palonosetron, alosetron, azasetron, bemesetron, zatisetron, batanopirde, MDL-73147EF; Metoclopramide, N-3389 (endo-3,9-dimethyl-3,9-diazabicyclo[3,3,1]non-7-yl-1H-indazole-3-carboxamide dihydrochloride), Y-25130 hydrochloride, MDL 72222, Tropanyl-3,5-dimethylbenzoate, 3-(4-Allylpiperazin-1-yl)-2-quinoxalinecarbonitrile maleate, zacopride hydrochloride, and mirtazepine. Other non-limiting examples of 5HT3-receptor antagonists (serotonin receptor antagonists) include: cilansetron, clozapine, cyproheptadine, dazopride, hydroxyzine, lerisetron, metoclopramide, mianserin, olanzapine, palonosetron (+ netupitant), quetiapine, qamosetron, ramosteron, ricasetron, risperidone, ziprasidone, and zatosetron.

In certain embodiments, the 5HT-3-receptor antagonist is granisetron, dolasetron, ondansetron hydrochloride, tropisctron, ramosctron, palonosctron, alosctron, bemesctron, zatisctron, batanopirde, MDL-73147EF, metoclopramide, N-3389, Y-25130 hydrochloride, MDL 72222, tropanyl-3,5-dimethylbenzoate 3-(4-Allyl-piperazin-1-yl)-2-quinoxalinecarbonitrile maleate, zacopride hydrochloride and mirtazepine.

In certain embodiments, the 5HT-3-receptor antagonist is granisetron, dolasetron, ondansetron hydrochloride, tropisetron, ramosetron, palonosetron, alosetron, bemesetron, and zatisetron.

In certain embodiments, the 5HT-3-receptor antagonist is granisetron, dolasetron and ondansetron.

In certain embodiments, the 5HT-3-receptor antagonist is granisetron.

In certain embodiments, the 5HT-3-receptor antagonist is ondansetron.

In some embodiments, the anti-emetic agent is an antihistamine. Non-limiting examples of antihistamines include: piperazine derivatives (e.g., cyclizine, meclizine, and cinnarizine); promethazine; dimenhydrinate (Dramamine, Gravol); diphenhydramine; hydroxyzine; buclizine; and meclizine hydrochloride (Bonine, Antivert), doxylamine, and mirtazapine.

In some embodiments, the anti-emetic agent is an anticholinergic agent (inhibitors of the acetylcholine receptors). Non-limiting examples of anticholinergic agents include: atropine, scopolamine, glycopyrron, hyoscine, artane (trihexy-5 trihexyphenidyl hydrochloride), cogentin (benztropine mesylate), akineton (biperiden hydrochloride), disipal (norflex orphenadrine citrate), diphenhydramine, hydroxyzine, hyoscyamine, and kemadrin (procyclidine hydrochloride).

In some embodiments, the anti-emetic agent is a steroid (e.g., a corticosteroid). Non-limiting examples of steroids include: betamethasone, dexamethasone, methylprednisolone, Prednisone®, and trimethobenzamide (Tigan).

In some embodiments, the anti-emetic agent is an NK1-receptor antagonists (e.g., Neurokinin 1 substance P receptor antagonists). Non-limiting examples of NK1-receptor antagonists include: aprepitant, casopitant, ezlopitant, fosaprepitant, maropitant, netupitant, rolapitant, and vestipitant.

Other non-limiting examples of NK1-receptor antagonists include: MPC-4505, GW597599, MPC-4505, GR205171, L-759274, SR 140333, CP-96,345, BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK 333A, YM-49244, YM-44778, ZM-274773, MEN-10930, S-19752, Neuronorm, YM-35375, DA-5018, MK-869, L-754030, CJ=11974, L-758298, DNK-33A, 6b-1, CJ=11974 j. Benserazide and carbidopak. TAK-637 [(aR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthyridine-6,13-dione], PD 154075, ([(2-benzofuran)-CH₂OCO]-(R)-alpha-MeTrp-(S)-NHCH(CH₃) Ph), FK888, and (D-Pro4, D-Trp7,9,10, Phe11) SP4-11.

In some embodiments, the anti-emetic agent is an anti-dopaminergic agents/dopamine receptor antagonist (e.g., dopamine receptor antagonist, e.g., D2 or D3 antagonists). Non-limiting examples include phenothiazines (e.g., promethazine, chlorpromazine, prochlorperazine, perphenazine, hydroxyzine, thiethylperazine, metopimazine); benzamides (e.g., metoclopramida, domperidone), butyrophenones (e.g., haloperidol, droperidol); alizapride, bromopride, clebopride, domperidone, itopride, metoclopramide, trimethobenzamide, and amisulpride.

In some embodiments, the anti-emetic agent is a non-psychoactive cannabinoids (e.g., Cannabidiol (CBD), Cannabidiol dimethylheptyl (CBD-DMH), Tetra-hydro-cannabinol (THC), Cannabinoid agonists such as WIN 55-212 (a CB1 and CB2 receptor agonist), Dronabinol (Marinol®), and Nabilone (Cesamet)).

Other exemplary anti-emetic agents include: c-9280 (Merck); benzodiazepines (diazepam, midazolam, lorazepam); neuroleptics/anti-psychotics (e.g., dixyrazine, haloperidol, and Prochlorperazine (Compazine®)); cerium oxalate; propofol; sodium citrate; dextrose; fructose (Nauzene); orthophosphoric acid; fructose; glucose (Emetrol); bismuth subsalicylate (Pepto Bismol); ephedrine; vitamin B6; peppermint, lavender, and lemon essential oils; and ginger.

Still other exemplary anti-emetic agents include those disclosed in US20120101089A1; U.S. Pat. Nos. 10,071,088 B2; 6,673,792 B1; 6,197,329 B1; 10,828,297 B2; 10,322,106 B2; 10,525,033 B2; WO 2009080351 A1; WO 2019203753 A2; WO 2002020001 A2; U.S. Pat. No. 8,119,697 B2; U.S. Pat. No. 5,039,528; US20090305964A1; and WO 2006/111169, each of which is incorporated by reference in its entirety.

In some embodiments, the additional therapeutic agent or regimen is administered to the patient prior to contacting with or administering the compounds and pharmaceutical compositions (e.g., about one hour prior, or about 6 hours prior, or about 12 hours prior, or about 24 hours prior, or about 48 hours prior, or about 1 week prior, or about 1 month prior).

In some embodiments, the additional therapeutic agent or regimen is administered to the patient at about the same time as contacting with or administering the compounds and pharmaceutical compositions. By way of example, the additional therapeutic agent or regimen and the compounds and pharmaceutical compositions are provided to the patient simultaneously in the same dosage form. As another example, the additional therapeutic agent or regimen and the compounds and pharmaceutical compositions are provided to the patient concurrently in separate dosage forms.

### Patient Selection

In some embodiments, the methods described herein further include the step of identifying a patient (e.g., a subject) in need of such treatment (e.g., by way of blood assay, body mass index, or other conventional method known in the art).

In some embodiments, the methods described herein further include the step of identifying a patient (e.g., patient) that has type 2 diabetes mellitus. In some embodiments, determining if the patient has type 2 diabetes mellitus includes performing an assay to determine the level of hemoglobin Alc (HbA1c), fasting plasma glucose, non-fasting plasma glucose, or any combination thereof. In some embodiments, the level of HbA1c is about 6.5% to about 24.0%. In some embodiments, the level of HbA1c is greater than or about 6.5%. In some embodiments, the level of HbA1c is greater than or about 8.0%. In some embodiments, the level of HbA1c is greater than or about 10.0%. In some embodiments, the level of HbA1c is greater than or about 12.0%. In some embodiments, the level of HbA1c is greater than or about 14.0%. In some embodiments, the level of HbA1c is greater than or about 16.0%. In some embodiments, the level of HbA1c is greater than or about 18.0%. In some embodiments, the level of HbA1c is greater than or about 20.0%. In some embodiments, the level of HbA1c is greater than or about 22.0%. In some embodiments, the level of HbA1c is greater than or about 24.0%.

In some embodiments, the level of fasting plasma glucose is greater than or about 120 mg/dL to greater than or about 750 mg/dL. In some embodiments, the level of fasting plasma glucose is greater than or about 200 mg/dL to greater than or about 500 mg/dL. In some embodiments, the level of fasting plasma glucose is greater than or about 300 mg/dL to greater than or about 700 mg/dL.

In some embodiments, the level of non-fasting plasma glucose is greater than or about 190 mg/dL to greater than or about 750 mg/dL. In some embodiments, the level of non-fasting plasma glucose is greater than or about 250 mg/dL to greater than or about 450 mg/dL. In some embodiments, the level of non-fasting plasma glucose is greater than or about 400 mg/dL to greater than or about 700 mg/dL.

In some embodiments, determining if the patient has type 2 diabetes mellitus further includes determining the patient's BMI. In some embodiments, the BMI of the patient is greater than or about 22 kg/m2 to greater than or about 100 kg/m2. In some embodiments, the BMI of the patient is greater than or about 30 kg/m2 to greater than or about 90 kg/m². In some embodiments, the BMI of the patient is greater than or about 40 kg/m2 to greater than or about 80 kg/m². In some embodiments, the BMI of the patient is greater than or about 50 kg/m2 to greater than or about 70 kg/m2.

In some embodiments, additional factors (e.g. risk factors) used for determining if the patient has type 2 diabetes mellitus further includes age and ethnicity of the patient. In some embodiments, the patient's age is greater than or about 10 years. In some embodiments, the patient's age is greater than or about 15 years. In some embodiments, the patient's age is greater than or about 20 years. In some embodiments, the patient's age is greater than or about 25 years. In some embodiments, the patient's age is greater than or about 30 years. In some embodiments, the patient's age is greater than or about 35 years. In some embodiments, the patient's age is greater than or about 40 years. In some embodiments, the patient's age is greater than or about 42 years. In some embodiments, the patient's age is greater than or about 44 years. In some embodiments, the patient's age is greater than or about 46 years. In some embodiments, the patient's age is greater than or about 48 years. In some embodiments, the patient's age is greater than or about 50 years. In some embodiments, the patient's age is greater than or about 52 years. In some embodiments, the patient's age is greater than or about 54 years. In some embodiments, the patient's age is greater than or about 56 years. In some embodiments, the patient's age is greater than or about 58 years. In some embodiments, the patient's age is greater than or about 60 years. In some embodiments, the patient's age is greater than or about 62 years. In some embodiments, the patient's age is greater than or about 64 years. In some embodiments, the patient's age is greater than or about 66 years. In some embodiments, the patient's age is greater than or about 68 years. In some embodiments, the patient's age is greater than or about 70 years. In some embodiments, the patient's age is greater than or about 72 years. In some embodiments, the patient's age is greater than or about 74 years. In some embodiments, the patient's age is greater than or about 76 years. In some embodiments, the patient's age is greater than or about 78 years. In some embodiments, the patient's age is greater than or about 80 years. In some embodiments, the patient's age is greater than or about 85 years. In some embodiments, the patient's age is greater than or about 90 years. In some embodiments, the patient's age is greater than or about 95 years. In some embodiments, the ethnicity of the patient may be African American, American Indian or Alaska Native, Asian American, Hispanics or Latinos, or Native Hawaiian or Pacific Islander.

### Synthesis of the Compounds

The compounds of this disclosure can be prepared from readily available starting materials using, for example, the following general methods, and procedures. It will be appreciated that where certain process conditions (i.e., reaction temperatures, times, mole ratios of reactants, solvents, pressures, etc.) are given, other process conditions can also be used unless otherwise stated. Optimum reaction conditions may vary with the reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting certain functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of this disclosure may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance CA USA), EMKA-Chemie Gmbh & Co. KG (Eching Germany), or Millipore Sigma (Burlington MA USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

Scheme I illustrates a general method which can be employed for the synthesis of compounds described herein, wherein each of A, ring B, L¹, L², R¹, R³, R⁴, and R⁵ are independently as defined herein.

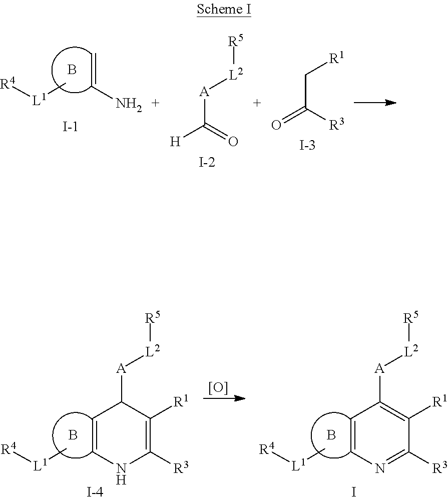

A Hantzsch style pyridine synthesis strategy can be used to synthesize the multi-substituted pyridine-based compound I-4. As shown in Scheme I, coupling compound I-1 with compounds I-2 and I-3 provides I-4. Oxidation of I-4, such as by CAN or DDQ, provides for compounds of Formula I. In some embodiments, the coupling reaction is performed under heated conditions in a suitable solvent (e.g., ethanol). In some embodiments, the oxidation step is performed under microwave irradiation.

Alternative strategies known in the art may be used to form ring B as needed. For example, Scheme II illustrates methods to synthesize compounds of Formula I-10, wherein each of A, L¹, L², R¹, R³, R⁴, and R⁵ are independently as defined herein, m is 0 or 1, and R⁵⁰ is an alkyl or substituted alkyl.

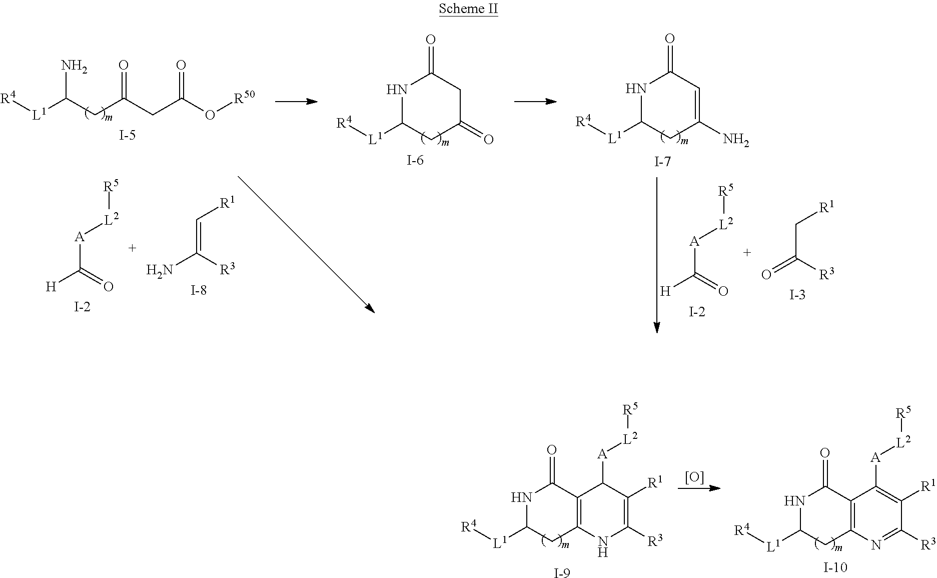

Compound I-5 can undergo cyclization under suitable conditions to provide compound I-6, which can be further reduced to provide compound I-7. Compound I-7 may then be coupled with compound I-2 and compound I-3 using the same Hantzsch style pyridine synthesis strategy as shown in Scheme I to provide compound I-9. Alternatively, compound I-5 can be coupled directly with compound I-2 and compound I-8 to provide compound I-9. Oxidation of compound I-9 in the presence of CAN or DDQ under suitable conditions (i.e., at elevated temperatures and/or under microwave irradiation) provides compounds of Formula I-10. In some embodiments, the nitrogen on I-5 may be protected and deprotected in later steps as needed using conventional methods.

Additionally, compound of Formula I-17 may be prepared as shown in Scheme III, wherein each of A, L¹, L², R¹, R³, R⁴, and R⁵ are independently as defined herein, and LG represents a suitable leaving group (such as halo, hydroxy, or triflate).

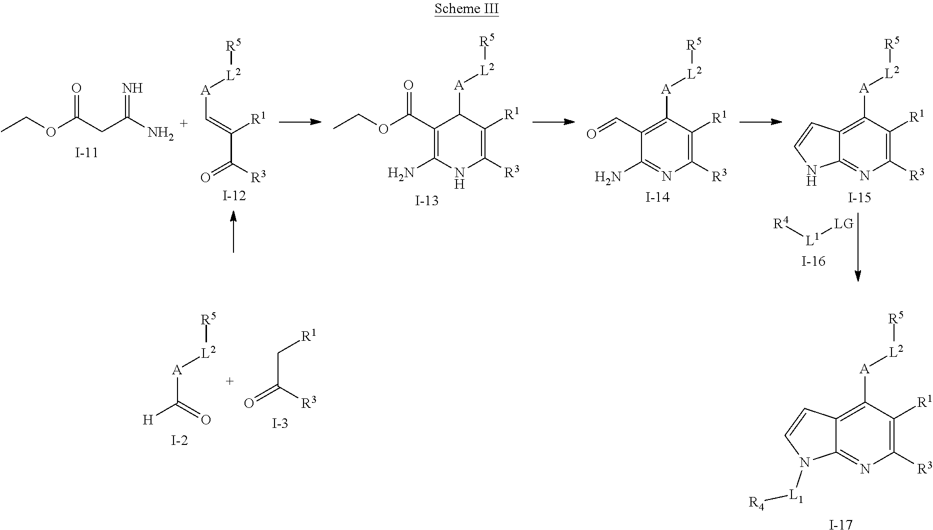

Compound I-12 may be prepared by condensation of compound I-2 with compound I-3. Compound I-12 can then undergo Guareschi-Thorpe type condensation with I-11 to provide compound I-13. Oxidation of compound I-13 in the presence of CAN or DDQ, followed by reduction of the ester provides compound I-14. Compound I-14 can cyclize in the presence of a methylating agent such as diazomethane or trimethylsilyldiazomethane to provide compound I-15. Alkylation of I-15 with I-16 in the presence of a base provides compounds of Formula I-17.

For any compound shown in Scheme I, II, or III, it should be understood that various derivatives can be provided by functional group interconversion at any step. In some embodiments, the various substituents of the compounds (e.g., A, L¹, L², R¹, R³, R⁴, and R⁵) are as defined herein. However, derivatization of compounds prior to reacting in any step, and/or further derivatization of the resulting reaction product, provides various compounds of Formula I. Appropriate starting materials and reagents can be purchased or prepared by methods known to one of skill in the art.

Upon each reaction completion, each of the intermediates or final compounds can be recovered, and optionally purified, by conventional techniques such as neutralization, extraction, precipitation, chromatography, filtration, and the like. In certain embodiments, when control of stereochemistry is desired, proper control of reaction conditions and selection of substituents for the reagents can at least partially dictate or preserve the formation of the various stereoisomers. Other modifications to arrive at compounds of this disclosure are within the skill of the art.

### General Synthesis

Typical embodiments of compounds described herein may be synthesized using the general reaction schemes described below. It will be apparent given the description herein that the general schemes may be altered by substitution of the starting materials with other materials having similar structures to result in products that are correspondingly different. Descriptions of syntheses follow to provide numerous examples of how the starting materials may vary to provide corresponding products. Given a desired product for which the substituent groups are defined, the necessary starting materials generally may be determined by inspection. Starting materials are typically obtained from commercial sources or synthesized using published methods. For synthesizing compounds which are embodiments described in the present disclosure, inspection of the structure of the compound to be synthesized will provide the identity of each substituent group. The identity of the final product will generally render apparent the identity of the necessary starting materials by a simple process of inspection, given the examples herein. In general, compounds described herein are typically stable and isolatable at room temperature and pressure.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Abbreviations (as Used Herein):

| | |
|---|---|
| ACN/MeCN | acetonitrile |
| AcOH/HOAc | acetic acid |
| ⁿBuLi | n-butyllithium |
| (Boc)₂O | di-tert-butyl dicarbonate |
| CAN | ceric ammonium nitrate |
| CataCXium A | di(1-adamantyl)-n-butylphosphine |
| CDI | carbonyldiimidazole |
| CHCl₃ | chloroform |
| DCC | N,N′-dicyclohexylcarbodiimide |
| DCM | dichloromethane |
| DIBAL-H | diisobutylaluminium hydride |
| DIEA | N,N-diisopropylethylamine |
| DMA | dimethylacetamide |
| DMAP | 4-dimethylaminopyridine |
| DMF | N,N-dimethylformamide |
| EA/EtOAc | ethyl acetate |
| EtOH | ethanol |
| FA | formic acid |
| HATU | 1-[bis(dimethylamino)methylene]-1H-1,2,3-triazolo[4,5- |
| | b]pyridinium 3-oxide hexafluorophosphate |
| HCl | hydrochloric acid |
| IBX | 2-iodoxybenzoic acid |
| MeI | methyl iodide |
| MeOH | methanol |
| NBS | N-bromosuccinimide |
| Pd/C | palladium on carbon |
| Pd(dba)₃ | tris(dibenzylideneacetone)dipalladium(0) |
| Pd(dppf)Cl₂ | bis(diphenylphosphino)ferrocene)palladium(II) dichloride |
| PE | petroleum ether |
| ⁱPrOH | isopropanol |
| PyBOP | (benzotriazol-1-yloxy)tripyrrolidinophosphonium |
| | hexafluorophosphate |
| RuPhos | 2-dicyclohexylphosphino-2,6-diisopropoxybiphenyl |
| TEA | triethylamine |
| TFA | trifluoroacetic acid |
| THF | tetrahydrofuran |
| TMSCHN₂ | trimethylsilyldiazomethane |
| XantPhos | (9,9-dimethyl-9H-xanthene-4,5- |
| | diyl)bis(diphenylphosphane) |
| Yb(OTf)₂ | ytterbium(III) trifluoromethanesulfonate |

General information: All evaporations or concentrations were carried out in vacuo with a rotary evaporator. Analytical samples were dried in vacuo (1-5 mm Hg) at rt. Thin layer chromatography (TLC) was performed on silica gel plates, spots were visualized by UV light (214 and 254 nm). Purification by column and flash chromatography was carried out using silica gel (100-200 mesh). Solvent systems were reported as mixtures by volume. NMR spectra were recorded on a Bruker 400 or Varian (400 MHz) spectrometer. ¹H chemical shifts are reported in δ values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on SHIMADZU LC20-MS2020 or Agilent 1260 series 6125B mass spectrometer or Agilent 1200 series, 6110 or 6120 mass spectrometer with electrospray ionization and excepted as otherwise indicated.

### Example I1: 4-((5-chloropyridin-2-yl)methyl)-2,2-difluoro-3-oxo-3,4-dihydro-2H-benzo[b][1,4]oxazine-7-carbaldehyde

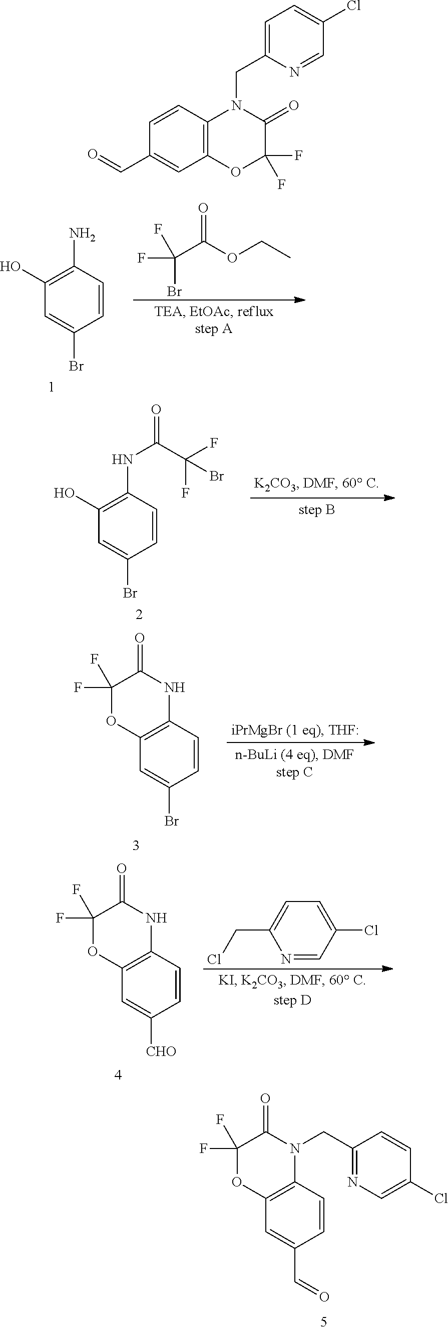

### Step A: 2-bromo-N-(4-bromo-2-hydroxyphenyl)-2,2-difluoroacetamide

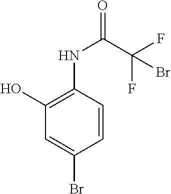 [SMILES: O=C(Nc1ccc(Br)cc1O)C(F)(F)Br]

A mixture of 2-amino-5-bromophenol (40 g, 212.74 mmol), Et₃N (23.68 g, 234.02 mmol, 32.57 mL), ethyl 2-bromo-2,2-difluoroacetate (60.46 g, 297.84 mmol, 38.38 mL) in EtOAc (400 mL) was degassed and purged with N₂ for 3 times, and then the mixture was stirred at 85° C. for 2 hrs under N₂ atmosphere. The reaction mixture was diluted with water (400 mL) and extracted with EA (200 mL*2), dried over Na₂SO₄, filtered and concentrated under reduced pressure to give a residue. The residue was purified by flash silica gel chromatography (Eluent of 0~100% EA/PE) to afford 2-bromo-N-(4-bromo-2-hydroxyphenyl)-2,2-difluoroacetamide (57 g, 77.68%) as a brown solid. ¹H NMR (400 MHz, CDCl₃) δ 7.95-7.98 (m, 2H), 7.00-7.03 (m, 2H).

### Step B: 7-bromo-2,2-difluoro-4H-1,4-benzoxazin-3-one

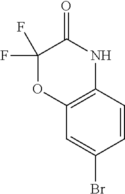 [SMILES: O=C1Nc2ccc(Br)cc2OC1(F)F]

To a solution of 2-bromo-N-(4-bromo-2-hydroxy-phenyl)-2,2-difluoro-acetamide (31 g, 89.87 mmol) in DMF (500 mL) was added K₂CO₃ (24.84 g, 179.74 mmol). The mixture was stirred at 60° C. for 2 hrs. The residue was diluted with H₂O (2500 mL) and extracted with EA (200 mL*3). The combined organic layers were washed with brine (300 mL*2), dried over Na₂SO₄, filtered and concentrated under reduced pressure to give a residue. The residue was purified by column chromatography (SiO₂, PE/EA=3/1 to 1/1) to afford 7-bromo-2,2-difluoro-4H-1,4-benzoxazin-3-one (21.7 g, 91.45%) as a yellow solid. ¹H NMR (400 MHz, CDCl₃) δ 9.80 (br s, 1H), 7.31 (d, J=2.0 Hz, 1H), 7.25 (dd, J=8.4, 2.0 Hz, 1H), 6.89 (d, J=8.4 Hz, 1H).

### Step C: 2,2-difluoro-3-oxo-4H-1,4-benzoxazine-7-carbaldehyde

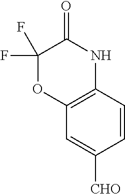 [SMILES: O=Cc1ccc2c(c1)OC(F)(F)C(=O)N2]

To a solution of 7-bromo-2,2-difluoro-4H-1,4-benzoxazin-3-one (8 g, 30.30 mmol) in THF (200 mL) under N₂ was added chloro(isopropyl)magnesium (2.8 M, 11.90 mL) dropwise at −10° C. After stirring for 30 min, the mixture was cooled to −30° C. and n-BuLi (2.5 M, 48.48 mL) was added dropwise over 15 min. After stirring at this temperature for 30 min, DMF (8.86 g, 121.20 mmol, 9.32 mL) was added and the mixture was stirred at 25° C. for 1.5 hrs under N₂ atmosphere. The reaction mixture was quenched by addition of sat. aqueous NH₄Cl (50 mL), and then diluted with H₂O 100 mL and extracted with EA (150 mL*3). The combined organic layers were washed with brine (150 mL), dried over Na₂SO₄, filtered and concentrated under reduced pressure to give a residue. The residue was purified by column chromatography (SiO₂, PE/EA=5/1 to 1/1) to afford 2,2-difluoro-3-oxo-4H-1,4-benzoxazine-7-carbaldehyde (4.48 g, 69.37%) as a yellow solid. ¹H NMR (400 MHz, (400 MHz, CDCl₃) δ 9.88 (s, 1H), 7.63-7.72 (m, 2H), 7.11 (d, J=8.4 Hz, 1H).

### Step D: 4-((5-chloropyridin-2-yl)methyl)-2,2-difluoro-3-oxo-3,4-dihydro-2H-benzo[b][1,4]oxazine-7-carbaldehyde

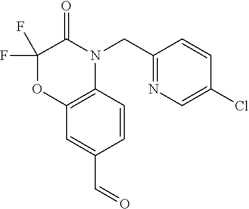 [SMILES: O=Cc1ccc2c(c1)OC(F)(F)C(=O)N2Cc1ccc(Cl)cn1]

To a solution of 2,2-difluoro-3-oxo-4H-1,4-benzoxazine-7-carbaldehyde (3 g, 14.08 mmol), 5-chloro-2-(chloromethyl)pyridine (2.51 g, 15.48 mmol) in DMF (30 mL) was added K₂CO₃ (5.84 g, 42.23 mmol), KI (2.34 g, 14.08 mmol). The mixture was stirred at 60° C. for 2 hrs. The residue was diluted with H₂O (150 mL) and extracted with EtOAc (100 mL*2). The combined organic layers were washed with brine (150 mL), dried over Na₂SO₄, filtered and concentrated under reduced pressure to give a residue. The residue was purified by column chromatography (SiO₂, PE/EA=3/1 to 1/1) to afford 4-((5-chloropyridin-2-yl)methyl)-2,2-difluoro-3-oxo-3,4-dihydro-2H-benzo[b][1,4]oxazine-7-carbaldehyde (1.88 g, 39.44%) as a yellow oil. LC-MS: m/z 339.0 (M+H)⁺. ¹H NMR (400 MHz, CDCl₃) δ 9.93 (s, 1H), 8.53 (d, J=2.4 Hz, 1H), 7.73 (d, J=1.6 Hz, 1H), 7.68-7.71 (m, 2H), 7.47 (d. J=8.4 Hz, 1H), 7.31 (d, J=8.4 Hz, 1H), 5.35 (s, 2H).

### Example I2: 4-((5-fluoropyrimidin-2-yl)methyl)-2,2-difluoro-3-oxo-3,4-dihydro-2H-benzo[b][1,4]oxazine-7-carbaldehyde

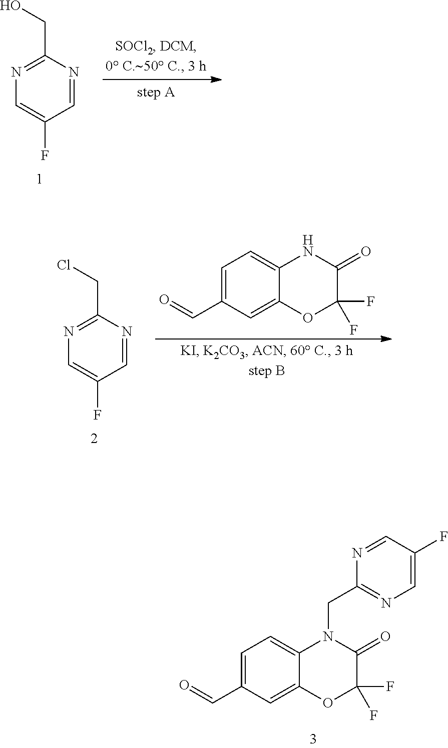 [SMILES: OCc1ncc(F)cn1 ; Fc1cnc(CCl)nc1 ; O=Cc1ccc2c(c1)OC(F)(F)C(=O)N2 ; O=Cc1ccc2c(c1)OC(F)(F)C(=O)N2Cc1ncc(F)cn1]

### Step A: 2-(chloromethyl)-5-fluoropyrimidine

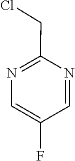

To a solution of (5-fluoropyrimidin-2-yl) methanol (45 g, 351.261 mmol) in DCM (600 mL) was added SOCl₂ (76.438 mL, 1053.782 mmol). The mixture was stirred at 50° C. for 3 hours. The reaction mixture was concentrated under vacuum to afford 2-(chloromethyl)-5-fluoropyrimidine (50 g, 82.56%) as a yellow oil. LC-MS: m/z 147.0 (M+H)⁺.

### Step B: 4-((5-fluoropyrimidin-2-yl)methyl)-2,2-difluoro-3-oxo-3,4-dihydro-2H-benzo[b][1,4]oxazine-7-carbaldehyde

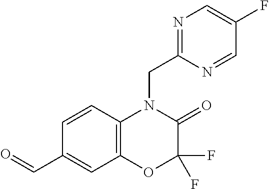 [SMILES: O=Cc1ccc2c(c1)OC(F)(F)C(=O)N2Cc1ncc(F)cn1]

To a solution of 2-(chloromethyl)-5-fluoropyrimidine (31.97 g, 218.166 mmol) in ACN (600 mL) were added 2,2-difluoro-3-oxo-3,4-dihydro-2H-benzo[b][1,4]oxazine-7-carbaldehyde (31 g, 145.444 mmol), potassium iodide (24.14 g, 145.444 mmol) and K₂CO₃ (80.40 g, 581.777 mmol). The mixture was stirred for 3 h at 60° C. under N₂. The mixture was poured into water (200 mL), extracted with ethyl acetate (200 mL×3). The combined organic layers were washed with brine (100 mL×3), dried over sodium sulfate, filtered and concentrated under vacuum. The crude product was stirred with a mixture of PE/EA (10/1, 200 mL) for 30 min, the mixture was filtered, the cake was dried under vacuum at 40° C. to afford 4-((5-fluoropyrimidin-2-yl)methyl)-2,2-difluoro-3-oxo-3,4-dihydro-2H-benzo[b][1,4]oxazine-7-carbaldehyde (43.8 g, 88.51%) as a yellow solid. ¹H NMR (400 MHz, DMSO-d₆) δ 9.94 (s, 1H), 8.91 (d, J=0.6 Hz, 2H), 7.90 (d, J=1.6 Hz, 1H), 7.79 (dd, J=8.4, 1.6 Hz, 1H), 7.49 (d, J=8.4 Hz, 1H), 5.54 (s, 2H). ¹⁹F NMR (376 MHz, DMSO-d₆) δ −75.47, −139.22. LC-MS: m/z 324.2 (M+H)⁺.

The following compounds were synthesized following the similar route of Example I1 and Example I2 with appropriate starting materials.

| Name | Structure | LC-MS |
|---|---|---|
| 2,2-difluoro-4-((5-fluoropyridin-2- yl)methyl)-3-oxo-3,4-dihydro-2H- benzo[b][1,4]oxazine-7-carbaldehyde | 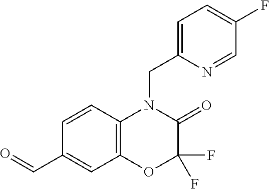 [SMILES: O=Cc1ccc2c(c1)OC(F)(F)C(=O)N2Cc1ccc(F)cn1] | LC-MS: m/z 323.1 (M + H)⁺ |
| 4-((5-chloro-3-methoxypyridin-2- yl)methyl)-2,2-difluoro-3-oxo-3,4-dihydro- 2H-benzo[b][1,4]oxazine-7-carbaldehyde | 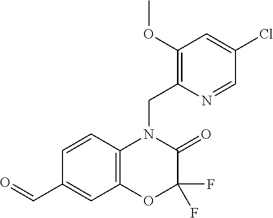 [SMILES: COc1cc(Cl)cnc1CN1C(=O)C(F)(F)Oc2cc(C=O)ccc21] | LC-MS: m/z 369.1 (M + H)⁺ |
| 2,2-difluoro-4-((5-fluoro-3-methoxypyridin- 2-yl)methyl)-3-oxo-3,4-dihydro-2H- benzo[b][1,4]oxazine-7-carbaldehyde | 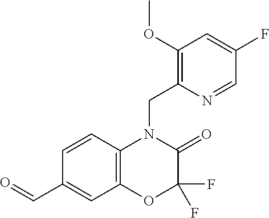 [SMILES: COc1cc(F)cnc1CN1C(=O)C(F)(F)Oc2cc(C=O)ccc21] | LC-MS: m/z 353.1 (M + H)⁺ |
| 2,2-difluoro-4-((5-fluoro-4- methoxypyrimidin-2-yl)methyl)-3-oxo-3,4- dihydro-2H-benzo[b][1,4]oxazine-7- carbaldehyde | 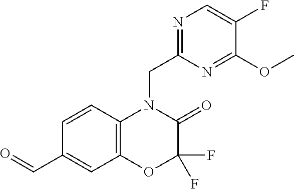 [SMILES: COc1nc(CN2C(=O)C(F)(F)Oc3cc(C=O)ccc32)ncc1F] | LC-MS: m/z 354.2 (M + H)⁺ |
| 4-((5-chloropyrimidin-2-yl)methyl)-2,2- difluoro-3-oxo-3,4-dihydro-2H- benzo[b][1,4]oxazine-7-carbaldehyde | 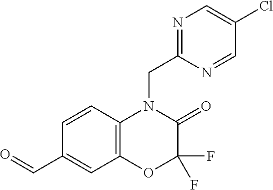 [SMILES: O=Cc1ccc2c(c1)OC(F)(F)C(=O)N2Cc1ncc(Cl)cn1] | LC-MS: m/z 340.2 (M + H)⁺ |
| 2-fluoro-4-((5-fluoropyrimidin-2- yl)methyl)-3-oxo-3,4-dihydro-2H- benzo[b][1,4]oxazine-7-carbaldehyde | 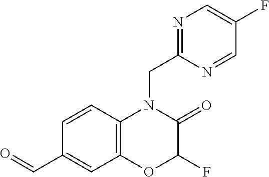 [SMILES: O=Cc1ccc2c(c1)OC(F)C(=O)N2Cc1ncc(F)cn1] | LC-MS: m/z 306.1 (M + H)⁺ |
| 4-((5-chloropyrimidin-2-yl)methyl)-2- fluoro-3-oxo-3,4-dihydro-2H- benzo[b][1,4]oxazine-7-carbaldehyde | 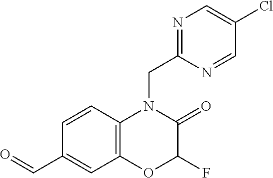 [SMILES: O=Cc1ccc2c(c1)OC(F)C(=O)N2Cc1ncc(Cl)cn1] | LC-MS: m/z 322.1 (M + H)⁺ |
| 2,2-difluoro-4-(1-(5-fluoropyrimidin-2- yl)ethyl)-3-oxo-3,4-dihydro-2H- benzo[b][1,4]oxazine-7-carbaldehyde | 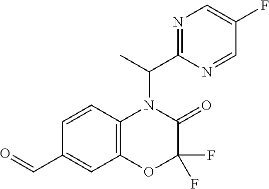 [SMILES: CC(c1ncc(F)cn1)N1C(=O)C(F)(F)Oc2cc(C=O)ccc21] | LC-MS: m/z 338.1 (M + H)⁺ |
| 2,2-difluoro-4-(1-(5-fluoropyrimidin-2-yl)- 2-methylpropyl)-3-oxo-3,4-dihydro-2H- benzo[b][1,4]oxazine-7-carbaldehyde | 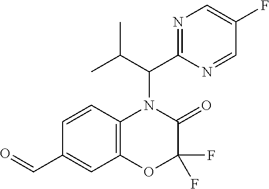 [SMILES: CC(C)C(c1ncc(F)cn1)N1C(=O)C(F)(F)Oc2cc(C=O)ccc21] | LC-MS: m/z 366.1 (M + H)⁺ |
| 4-(2-((tert-butyldimethylsilyl)oxy)-1-(5- fluoropyrimidin-2-yl)ethyl)-2,2-difluoro-3- oxo-3,4-dihydro-2H-benzo[b][1,4]oxazine- 7-carbaldehyde | 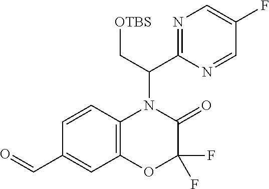 [SMILES: CC(C)(C)[Si](C)(C)OCC(c1ncc(F)cn1)N1C(=O)C(F)(F)Oc2cc(C=O)ccc21] | LC-MS: m/z 468.2 (M + H)⁺ |

### Example I3: 4-[(5-chloropyridin-2-yl)methyl]-2,2-dimethyl-3-oxo-3,4-dihydro-2H-benzo[b][1,4]oxazine-7-carbaldehyde

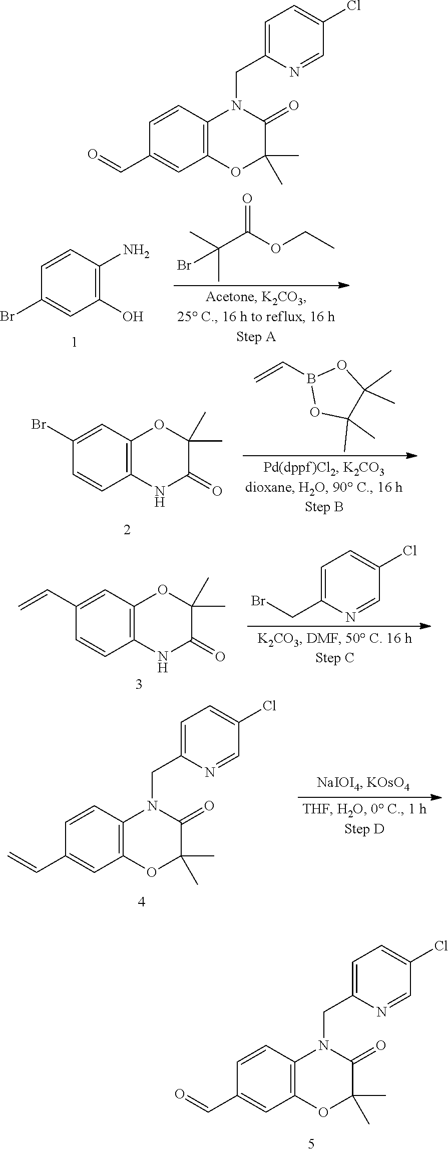

### Step A: 7-bromo-2,2-dimethyl-2H-benzo[b][1,4]oxazin-3(4H)-one

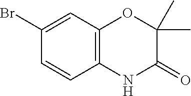

To a solution of 2-amino-5-bromophenol (15.0 g, 79.779 mmol) in Acetone (300 mL) were added ethyl 2-bromo-2-methylpropanoate (31.12 g, 159.557 mmol) and K₂CO₃ (33.08 g, 239.336 mmol), the mixture was stirred at rt for 16 hrs under N₂. And then stirred at 85° C. for another 16 hrs under N₂. The mixture was added to ice-water (500 mL), extracted with EA (500 mL×2), the combined organic layers were washed with brine (500 mL×2), dried over sodium sulfate, filtered and concentrated under vacuum. The resulting residue was stirred with EA/PE=1/10 to afford 7-bromo-2,2-dimethyl-2H-benzo[b][1,4]oxazin-3(4H)-one (23 g, 112.51%) as a brown solid. LC-MS: m/z 257.9 (M+H)⁺. ¹H NMR (400 MHz, DMSO-d₆) δ 10.81 (m, 1H), 7.12-7.14 (m, 2H), 6.87 (dd, J=5.6, 4.0 Hz, 1H), 1.38 (s, 6H).

### Step B: 2,2-dimethyl-7-vinyl-3,4-dihydro-2H-benzo[b][1,4]oxazin-3-one

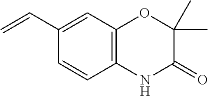

To a solution of 7-bromo-2,2-dimethyl-3,4-dihydro-2H-benzo[b][1,4]oxazin-3-one (20.00 g, 78.094 mmol) in dioxane (500 mL) and H₂O (50 mL) were added 4,4,5,5-tetramethyl-2-vinyl-1,3,2-dioxaborolane (24.06 g, 156.189 mmol), Pd(dppf)Cl₂·CH₂Cl₂ (3.19 g, 3.905 mmol) and K₂CO₃ (32.38 g, 234.283 mmol), the reaction mixture was stirred at 90° C. for 18 hrs under N₂. The mixture was concentrated in vacuum, the resulting residue was purified by silica gel chromatography (eluting PE/EA=5/1) to afford 2,2-dimethyl-7-vinyl-3,4-dihydro-2H-benzo[b][1,4]oxazin-3-one (12.33 g, 77.68%) as a pink solid. LC-MS: m/z 204.3 (M+H)⁺.

### Step C: 4-[(5-chloropyridin-2-yl)methyl]-2,2-dimethyl-7-vinyl-3,4-dihydro-2H-benzo[b][1,4]oxazin-3-one

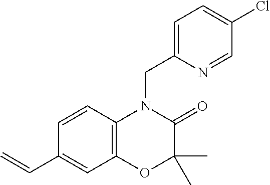 [SMILES: C=Cc1ccc2c(c1)OC(C)(C)C(=O)N2Cc1ccc(Cl)cn1]

To a solution of 2,2-dimethyl-7-vinyl-3,4-dihydro-2H-benzo[b][1,4]oxazin-3-one (5.53 g, 27.209 mmol) in DMF (150 mL) were added K₂CO₃ (18.80 g, 136.046 mmol), followed by 2-(bromomethyl)-5-chloropyridine (8.43 g, 40.814 mmol), the reaction mixture was stirred at 50° C. for 16 hrs under N₂. The mixture was added to water (80 mL), extracted with EA (50 mL×2), the combined organic layers were washed with brine (50 mL×2), dried over sodium sulfate, filtered and concentrated under vacuum. The resulting residue was purified by silica gel chromatography (eluting PE/EA=8/1) to afford 4-[(5-chloropyridin-2-yl)methyl]-2,2-dimethyl-7-vinyl-3,4-dihydro-2H-benzo[b][1,4]oxazin-3-one (4.3 g, 48.06%) as a yellow oil. LC-MS: m/z 329.2 (M+H)⁺. ¹H NMR (400 MHz, DMSO-d₆) δ 8.55 (d, J=2.0 Hz, 1H), 7.90 (dd, J=8.4, 2.4 Hz, 1H), 7.30 (d, J=8.4 Hz, 1H), 7.15 (d, J=1.6 Hz, 1H), 7.03 (dd, J=8.4, 1.6 Hz, 1H), 6.93 (d, J=8.4 Hz, 1H), 6.58-6.65 (m, 1H), 5.75 (d, J=17.2 Hz, 1H), 5.17-5.20 (m, 3H), 1.47 (s, 6H).

### Step D: 4-[(5-chloropyridin-2-yl)methyl]-2,2-dimethyl-3-oxo-3,4-dihydro-2H-benzo[b][1,4]oxazine-7-carbaldehyde

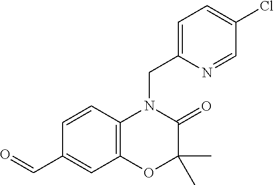 [SMILES: CC1(C)Oc2cc(C=O)ccc2N(Cc2ccc(Cl)cn2)C1=O]

To a solution of 4-[(5-chloropyridin-2-yl)methyl]-2,2-dimethyl-7-vinyl-3,4-dihydro-2H-benzo[b][1,4]oxazin-3-one (3.3 g, 10.036 mmol) in THF (100 mL) were added dipotassium dioxidodioxo-λ6-osmium(VI) dihydrate (0.37 g, 1.004 mmol) at 0° C., followed by a solution of NaIO₄ (8.59 g, 40.146 mmol) in H₂O (100 mL), the reaction mixture was stirred at 0° C. for 1 hr. To the mixture was added EA (100 mL), the mixture was washed with water (200 mL) and brine (100 mL), the organic layer was dried over sodium sulfate, filtered and concentrated under vacuum. The resulting residue was purified by silica gel chromatography (eluting PE/EA=6/1) to afford 4-[(5-chloropyridin-2-yl)methyl]-2,2-dimethyl-3-oxo-3,4-dihydro-2H-benzo[b][1,4]oxazine-7-carbaldehyde (2.6 g, 78.32%) as a yellow oil. LC-MS: m/z 331.2 (M+H)⁺. ¹H NMR (400 MHz, DMSO-d₆) δ 9.85 (s, 1H), 8.53 (d, J=2.4 Hz, 1H), 7.93 (dd, J=8.4, 2.4 Hz, 1H), 7.55 (dd, J=8.4, 1.5 Hz, 1H), 7.46 (d, J=2 Hz, 1H), 7.39 (d, J=8.4 Hz, 1H), 7.20 (d, J=8.4 Hz, 1H), 5.28 (s, 2H), 1.50 (s, 6H).

The following compounds were synthesized following the similar route of Example I3 with appropriate starting materials.

| Name | Structure | LC-MS |
|---|---|---|
| 4-[(5-cyanopyridin-2-yl)methyl]-2,2- dimethyl-3-oxo-3,4-dihydro-2H- benzo[b][1,4]oxazine-7-carbaldehyde | 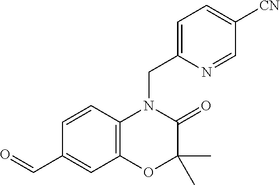 [SMILES: CC1(C)Oc2cc(C=O)ccc2N(Cc2ccc(C#N)cn2)C1=O] | LC-MS: m/z 322.2 (M + H)⁺ |
| 4-((5-fluoropyridin-2-yl)methyl)-2,2- dimethyl-3-oxo-3,4-dihydro-2H- benzo[b][1,4]oxazine-7-carbaldehyde | 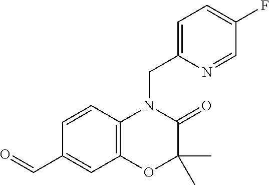 [SMILES: CC1(C)Oc2cc(C=O)ccc2N(Cc2ccc(F)cn2)C1=O] | LC-MS: m/z 314.2 (M + H)⁺ |
| 4-((5-fluoropyrimidin-2-yl)methyl)-2,2- dimethyl-3-oxo-3,4-dihydro-2H- benzo[b][1,4]oxazine-7-carbaldehyde | 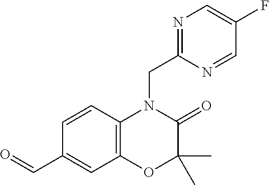 [SMILES: CC1(C)Oc2cc(C=O)ccc2N(Cc2ncc(F)cn2)C1=O] | LC-MS: m/z 316.1 (M + H)⁺ |
| 4-((5-chloropyrimidin-2-yl)methyl)-2,2- dimethyl-3-oxo-3,4-dihydro-2H- benzo[b][1,4]oxazine-7-carbaldehyde | 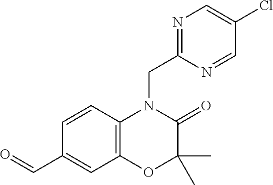 [SMILES: CC1(C)Oc2cc(C=O)ccc2N(Cc2ncc(Cl)cn2)C1=O] | LC-MS: m/z 332.0 (M + H)⁺ |
| 4-((6-formyl-2-oxobenzo[d]oxazol- 3(2H)-yl)methyl)benzonitrile | 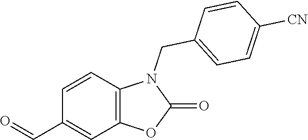 [SMILES: N#Cc1ccc(Cn2c(=O)oc3cc(C=O)ccc32)cc1] | LC-MS: m/z 279.1 (M + H)⁺ |
| 3-((5-chloropyridin-2-yl)methyl)-2-oxo- 2,3-dihydrobenzo[d]oxazole-6- carbaldehyde | 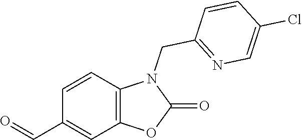 [SMILES: O=Cc1ccc2c(c1)oc(=O)n2Cc1ccc(Cl)cn1] | LC-MS: m/z 289.1 (M + H)⁺ |
| 3-((5-fluoropyridin-2-yl)methyl)-2-oxo- 2,3-dihydrobenzo[d]oxazole-6- carbaldehyde | 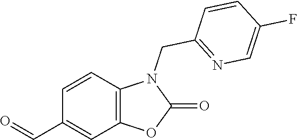 [SMILES: O=Cc1ccc2c(c1)oc(=O)n2Cc1ccc(F)cn1] | LC-MS: m/z 273.1 (M + H)⁺ |
| 6-((6-formyl-2-oxobenzo[d]oxazol- 3(2H)-yl)methyl)nicotinonitrile | 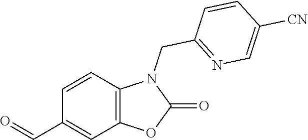 [SMILES: N#Cc1ccc(Cn2c(=O)oc3cc(C=O)ccc32)nc1] | LC-MS: m/z 197.0 (M + H)⁺ |
| 3-((5-fluoropyrimidin-2-yl)methyl)-2- oxo-2,3-dihydrobenzo\|d\|oxazole-6- carbaldehyde | 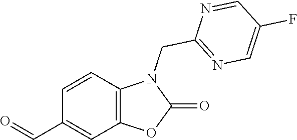 [SMILES: O=Cc1ccc2c(c1)oc(=O)n2Cc1ncc(F)cn1] | LC-MS: m/z 274.2 (M + H)⁺ |
| 4-((5-fluoropyridin-2-yl)methyl)-3-oxo- 3,4-dihydrospiro\|benzo[b][1,4]oxazine- 2,1′-cyclopropane]-7-carbaldehyde | 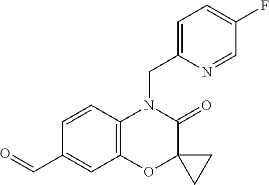 [SMILES: O=Cc1ccc2c(c1)OC1(CC1)C(=O)N2Cc1ccc(F)cn1] | LC-MS: m/z 313.1 (M + H)⁺ |
| 4-((5-chloropyrimidin-2-yl)methyl)-3- oxo-3,4- dihydrospiro[benzo[b][1,4]oxazine-2,1′- cyclobutane]-7-carbaldehyde | 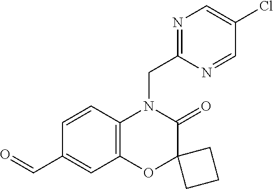 [SMILES: O=Cc1ccc2c(c1)OC1(CCC1)C(=O)N2Cc1ncc(Cl)cn1] | LC-MS: m/z 344.1 (M + H)⁺ |

### Example I4: 1-((5-chloropyrimidin-2-yl)methyl)-3-methyl-2-oxo-1,2-dihydroquinoxaline-6-carbaldehyde

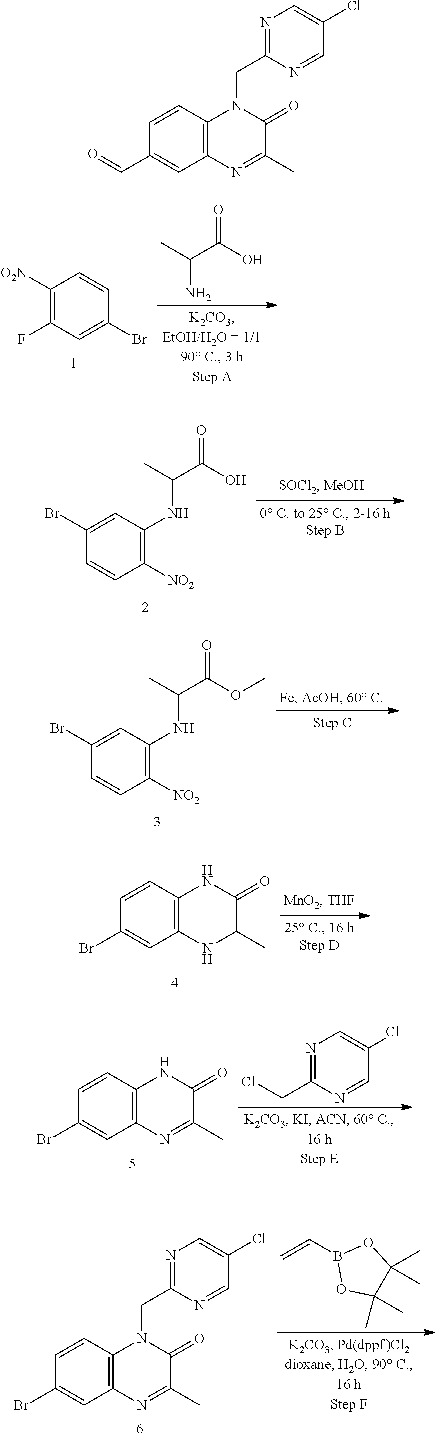

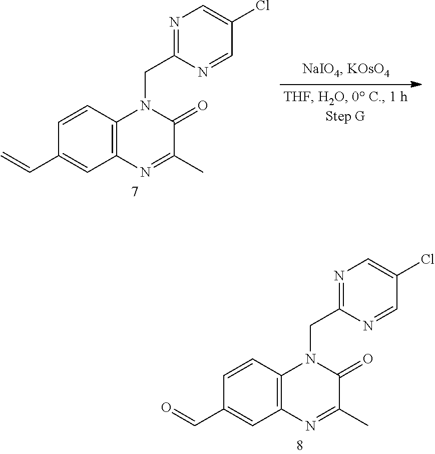 [SMILES: C=Cc1ccc2c(c1)nc(C)c(=O)n2Cc1ncc(Cl)cn1 ; Cc1nc2cc(C=O)ccc2n(Cc2ncc(Cl)cn2)c1=O]

### Step A: (5-bromo-2-nitrophenyl)alanine

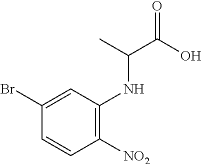 [SMILES: CC(Nc1cc(Br)ccc1[N+](=O)[O-])C(=O)O]

To a mixture of 4-bromo-2-fluoro-1-nitrobenzene (5 g, 22.73 mmol) and DL-alanine (2.02 g, 22.723 mmol) in EtOH (25 mL) and ILO (25 mL) were added K₂CO₃ (3.77 g, 27.273 mmol). The reaction mixture was stirred at 90° C. for 3 h. The mixture was cooled to 25° C. and adjusted to pH=2 by 1N HCl. The mixture was filtered. The filter cake was washed with water (10 mL) and dried under reduced pressure to afford (5-bromo-2-nitrophenyl)alanine (6600 mg, 90.41%) as a white solid. LC-MS: m/z 289.1 (M+H)⁺.

### Step B: methyl(5-bromo-2-nitrophenyl)alaninate

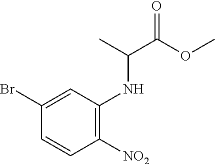 [SMILES: COC(=O)C(C)Nc1cc(Br)ccc1[N+](=O)[O-]]

To a solution of (5-bromo-2-nitrophenyl)alanine (6.6 g, 22.83 mmol) in MeOH (50 mL) was slowly added SOCl₂ (3.3 mL, 45.66 mmol) at 0° C. The reaction mixture was stirred at rt for 16 h. The mixture was concentrated under vacuum. The residue was added to EA (100 mL), washed with aq. NaHCO₃ (50 mL) and brine (30 mL), dried with sodium sulfate, filtered and concentrated under vacuum to afford methyl(5-bromo-2-nitrophenyl)alaninate (6200 mg, 89.59%) as a brown solid. LC-MS: m/z 303.1 (M+H)⁺.

### Step C: 6-bromo-3-methyl-3,4-dihydroquinoxalin-2 (1H)-one

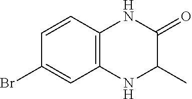

To a solution of methyl(5-bromo-2-nitrophenyl)alaninate (6 g, 19.78 mmol) in AcOH (60 mL) was added iron (11.0 g, 197.94 mmol). The reaction mixture was stirred at 60° C. for 3 h under N₂. The mixture was allowed to cooled to 25° C. and filtered. The filtrate was concentrated under vacuum. The residue was diluted with water (100 mL), extracted with EtOAc (100 mL×3), washed with brine (20 mL), dried with sodium sulfate, filtered and concentrated under vacuum. The residue was purified by silica gel chromatography (PE/EA=3/1) to afford 6-bromo-3-methyl-3,4-dihydroquinoxalin-2 (1H)-one (4000 mg, 83.89%) as a white solid. LC-MS: m/z 241.1 (M+H)⁺. ¹H NMR (400 MHz, DMSO-d₆) δ 10.29 (s, 1H), 6.81 (d, J=2.0 Hz, 1H), 6.72-6.75 (m, 1H), 6.83-6.85 (m, 1H), 6.27 (s, 1H), 3.78-3.84 (m, 1H), 1.23 (d, J=6.8 Hz, 3H).

### Step D: 4-[(5-chloropyridin-2-yl)methyl]-2,2-dimethyl-3-oxo-3,4-dihydro-2H-benzo[b][1,4]oxazine-7-carbaldehyde

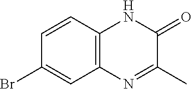

To a solution of 6-bromo-3-methyl-3,4-dihydroquinoxalin-2 (1H)-one (4 g, 16.59 mmol) in THF (60 mL) was added MnO₂ (7.64 g, 87.934 mmol). The reaction mixture was stirred at 25° C. for 16 h. The mixture was filtered, the filtrate was concentrated under vacuo to afford 6-bromo-3-methylquinoxalin-2(1H)-one (3600 mg, 90.76%) as a brown solid. LC-MS: m/z 239.1 (M+H)⁺.

### Step E: 6-bromo-1-((5-chloropyrimidin-2-yl)methyl)-3-methylquinoxalin-2 (1H)-one

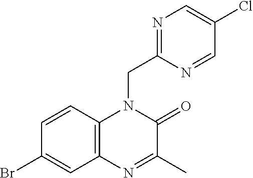 [SMILES: Cc1nc2cc(Br)ccc2n(Cc2ncc(Cl)cn2)c1=O]

A mixture of 6-bromo-3-methylquinoxalin-2(1H)-one (1 g, 4.18 mmol), 5-chloro-2-(chloromethyl)pyrimidine (1.02 g, 6.27 mmol), K₂CO₃ (2.89 g, 20.91 mmol) and potassium iodide (0.69 g, 4.18 mmol) in ACN (20 mL) was stirred at 60° C. for 16 h under N₂. The mixture was filtered. The filtrate was concentrated under vacuum. The residue was purified by silica gel chromatography (eluting PE/EA=3/1) to afford 6-bromo-1-((5-chloropyrimidin-2-yl)methyl)-3-methylquinoxalin-2 (1H)-one (980 mg, 64.08%) as a yellow solid. LC-MS: m/z 365.1 (M+H)⁺. ¹H NMR (400 MHz, DMSO-d₆) δ 8.88 (s, 2H), 7.97 (s, 1H), 7.62-7.64 (m, 1H), 7.34-7.36 (m, 1H), 5.67 (s, 2H), 2.48 (s, 3H).

### Step F: 1-((5-chloropyrimidin-2-yl)methyl)-3-methyl-6-vinylquinoxalin-2 (1H)-one

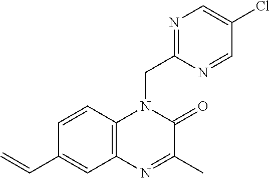 [SMILES: C=Cc1ccc2c(c1)nc(C)c(=O)n2Cc1ncc(Cl)cn1]

To a mixture of 6-bromo-1-((5-chloropyrimidin-2-yl)methyl)-3-methylquinoxalin-2 (1H)-one (980 mg, 2.680 mmol) and 4,4,5,5-tetramethyl-2-vinyl-1,3,2-dioxaborolane (1238 mg, 8.04 mmol) in dioxane (10 mL) and H₂O (1 mL) were added K₂CO₃ (1111 mg, 8.041 mmol) and Pd(dppf)Cl₂ (196 mg, 0.26 mmol). The reaction mixture was stirred at 90° C. for 16 h under N₂. The mixture was concentrated under vacuum and the residue was purified by silica gel chromatography (PE/EA=4/1) to afford 1-((5-chloropyrimidin-2-yl)methyl)-3-methyl-6-vinylquinoxalin-2 (1H)-one (330 mg, 39.36%) as a yellow solid. LC-MS: m/z 313.6 (M+H)⁺.

### Step G: 1-((5-chloropyrimidin-2-yl)methyl)-3-methyl-2-oxo-1,2-dihydroquinoxuline-6-carbaldehyde

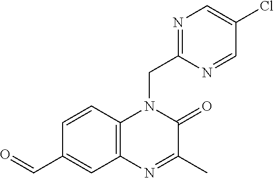 [SMILES: Cc1nc2cc(C=O)ccc2n(Cc2ncc(Cl)cn2)c1=O]

To a mixture of 1-((5-chloropyrimidin-2-yl)methyl)-3-methyl-6-vinylquinoxalin-2 (1H)-one (330 mg, 1.05 mmol) and dipotassium dioxidodioxo-λ6-osmium^{(VI)} dihydrate (40 mg, 0.11 mmol) in THF (10 mL) and H₂O (5 mL) was slowly added sodium periodate (902 mg, 4.22 mmol) at 0° C. The reaction mixture was stirred at 0° C. for 1 h. The mixture was diluted with water (15 mL), extracted with EA (15 mL×3), the combined organic layers were washed with brine (10 mL), dried with sodium sulfate, filtered and concentrated under vacuum. The resulting residue was purified by silica gel chromatography (PE/EA=1/1) to afford 1-((5-chloropyrimidin-2-yl)methyl)-3-methyl-2-oxo-1,2-dihydroquinoxaline-6-carbaldehyde (150 mg, 45.17%) as a yellow solid. LC-MS: m/z 315.2 (M+H)⁺.

### Example I5: 4-((5-chloropyridin-2-yl)methyl)-2-methyl-3-oxo-3,4-dihydropyrido[2,3-b]pyrazine-7-carbaldehyde

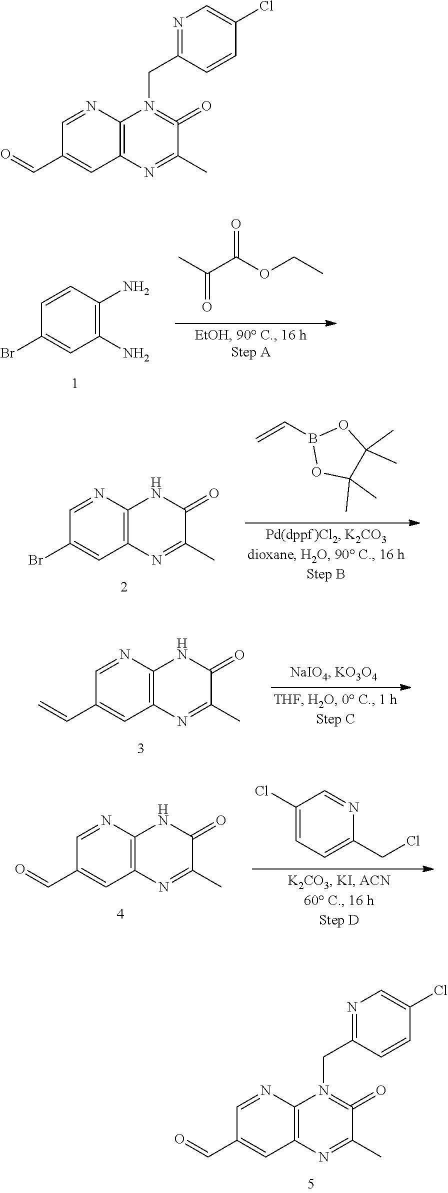

### Step A: 7-bromo-2-methylpyrido[2,3-b]pyrazin-3(4H)-one

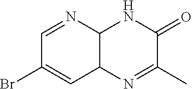

To a solution of 4-bromobenzene-1,2-diamine (11.60 g, 61.69 mmol) in EtOH (232 mL) was added ethyl 2-oxopropanoate (10.75 g, 92.54 mmol), the reaction mixture was stirred at 90° C. for 16 h under N₂. The mixture was filtered and the cake was dried under reduced pressure at 50° C. for 30 min to afford 7-bromo-2-methylpyrido[2,3-b]pyrazin-3(4H)-one (12.6 g, 85.48%) as a yellow solid. ¹H NMR (400 MHz, DMSO-d₆) δ 12.86 (s, 1H), 8.58 (d, J=2.4 Hz, 1H), 8.38 (d, J=2.4 Hz, 1H), 2.42 (s, 3H).

### Step B: 2-methyl-7-vinylpyrido[2,3-b]pyrazin-3(4H)-one

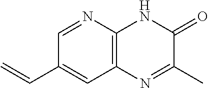

To a solution of 7-bromo-2-methylpyrido[2,3-b]pyrazin-3(4H)-one (406 mg, 1.699 mmol) in dioxane (30 mL) and H₂O (5 mL) were added 4,4,5,5-tetramethyl-2-vinyl-1,3,2-dioxaborolane (785 mg, 5.096 mmol), K₂CO₃ (704 mg, 5.096 mmol) and Pd(dppf)Cl₂ (124 mg, 0.1699 mmol), the reaction mixture was stirred at 90° C. for 16 h under N₂. The mixture was concentrated under vacuum and the resulting residue was purified by silica gel chromatography (eluting PE/EA=3/1) to afford 2-methyl-7-vinylpyrido[2,3-b]pyrazin-3(4H)-one (87 mg, 27.39%) as a white solid. LC-MS: m/z 188.0 (M+H)⁺.

### Step C: 2-methyl-3-oxo-3,4-dihydropyrido[2,3-b]pyrazine-7-carbaldehyde

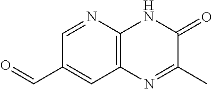

To a solution of 2-methyl-7-vinylpyrido[2,3-b]pyrazin-3(4H)-one (174 mg, 0.93 mmol) in THF (20 mL) were added a solution of NaIO₄ (796 mg, 3.71 mmol) in H₂O (20 mL) and dipotassium dioxidodioxo-λ⁶-osmium(VI) dihydrate (34.26 mg, 0,093 mmol) at 0° C., the reaction mixture was stirred at 0° C. for 1 h under N₂. The mixture was added to water (60 mL), extracted with EA (30 mL×2), the combined organic layers were dried over sodium sulfate, filtered and concentrated under vacuum to afford 2-methyl-3-oxo-3,4-dihydropyrido[2,3-b]pyrazine-7-carbaldehyde (122 mg, 70%) as a white solid. LC-MS: m/z 190.0 (M−H)⁺.

### Step D: 4-((5-chloropyridin-2-yl)methyl)-2-methyl-3-oxo-3,4-dihydropyrido[2,3-b]pyrazine-7-carbaldehyde

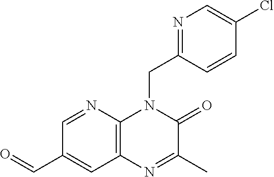 [SMILES: Cc1nc2cc(C=O)cnc2n(Cc2ccc(Cl)cn2)c1=O]

To a solution of 2-methyl-3-oxo-3,4-dihydropyrido[2,3-b]pyrazine-7-carbaldehyde (122 mg, 0.646 mmol) in ACN (10 mL) were added 5-chloro-2-(chloromethyl)pyridine (156.73 mg, 0.968 mmol), KI (107.06 mg, 0.646 mmol) and K₂CO₃ (356.51 mg, 2.580 mmol), the reaction mixture was stirred at 60° C. for 16 h under N₂. The mixture was added to water (30 mL), extracted with EA (30 mL×2), the combined organic layers were washed with brine (30 mL×3), dried over sodium sulfate, filtered and concentrated under vacuum. The resulting residue was purified by silica gel chromatography (eluting PE/EA=1/1) to afford 4-((5-chloropyridin-2-yl)methyl)-2-methyl-3-oxo-3,4-dihydropyrido[2,3-b]pyrazine-7-carbaldehyde (60 mg, 41.67%) as a yellow oil. LC-MS: m/z 315.2 (M+H)⁺.

### Example I6: 1-(1,1,1-trifluoro-3-methoxypropan-2-yl)-1H-imidazol-5-amine

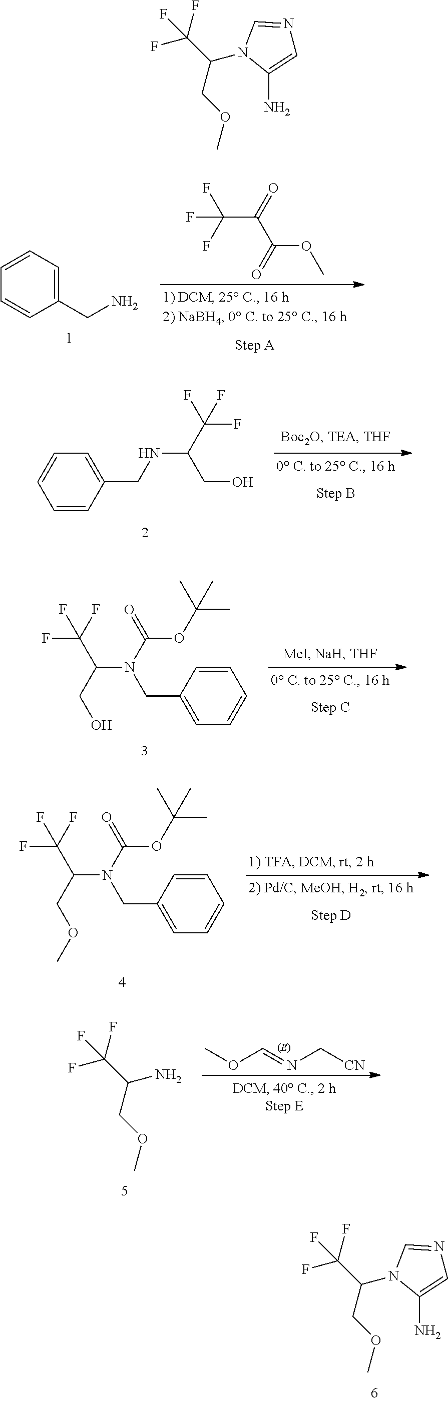

### Step A: 2-(benzylamino)-3,3,3-trifluoropropan-1-ol

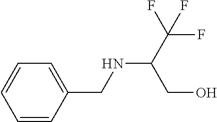 [SMILES: OCC(NCc1ccccc1)C(F)(F)F]

To a solution of phenylmethanamine (3.850 mL, 35.243 mmol) in DCM (100 mL) was added methyl 3,3,3-trifluoro-2-oxopropanoate (5000 mg, 32.039 mmol), the mixture was stirred at 25° C. for 16 h under N₂. NaBH₄ (2424.07 mg, 64.078 mmol) was added to the reaction mixture at 0° C. under N₂, the reaction mixture was stirred at 25° C. for 16 h. To the mixture was added water (100 mL), the mixture was separated and the aqueous phase was extracted with DCM (300 mL), the combined organic layers was washed with brine (50 mL), dried over sodium sulfate, filtered and concentrated under vacuum to afford 2-(benzylamino)-3,3,3-trifluoropropan-1-ol (7000 mg, 99.67%) as a yellow oil, which was used in the next step without further purification. LC-MS: m/z 220.4 (M−H)⁺.

### Step B: tert-butyl benzyl(1,1,1-trifluoro-3-hydroxypropan-2-yl)carbamate

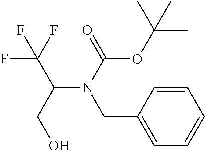 [SMILES: CC(C)(C)OC(=O)N(Cc1ccccc1)C(CO)C(F)(F)F]

To a solution of 2-(benzylamino)-3,3,3-trifluoropropan-1-ol (7000 mg, 31.933 mmol) in THF (100 mL) were added TEA (13.316 mL, 95.799 mmol), followed by Di-tert-butyl dicarbonate (10454.02 mg, 47.899 mmol) at 0° C. under N₂. The reaction mixture was stirred at 25° C. for 16 h under N₂. The mixture was concentrated under vacuum. The resulting residue was purified by silica gel chromatography (eluting PE/EA=25/1) to afford tert-butyl benzyl(1,1,1-trifluoro-3-hydroxypropan-2-yl)carbamate (2.5 g, 24.52%) as a yellow oil. LC-MS: m/z 342.4 (M+Na).

### Step C: tert-butyl benzyl(1,1,1-trifluoro-3-methoxypropan-2-yl)carbamate

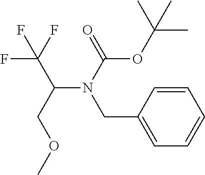 [SMILES: COCC(N(Cc1ccccc1)C(=O)OC(C)(C)C)C(F)(F)F]

To a solution of tert-butyl benzyl(1,1,1-trifluoro-3-hydroxypropan-2-yl)carbamate (2300 mg, 7.203 mmol) in THF (50 mL) was added NaH (576.22 mg, 14.406 mmol) at 0° C., the reaction mixture was stirred at 0° C. for 1 h under N₂, iodomethane (1.169 mL, 14.406 mmol) was added. The reaction mixture was stirred at 25° C. for 18 h under N₂. To the mixture was added water (100 mL), the residue was extracted with EA (100 mL×2). The combined organic layers were washed with brine (100 mL×2), dried over sodium sulfate, filtered and concentrated under vacuum. The resulting residue was purified by column chromatography (eluting PE/EA-5/1) to afford tert-butyl benzyl(1,1,1-trifluoro-3-methoxypropan-2-yl)carbamate (1400 mg, 58.31%) as a yellow oil. LC-MS: m/z 234.1 (M+H-Boc)⁺.

### Step D: 1,1,1-trifluoro-3-methoxypropan-2-amine

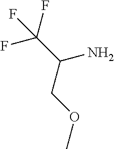

To a solution of tert-butyl benzyl(1,1,1-trifluoro-3-methoxypropan-2-yl)carbamate (200 mg, 0.600 mmol) in DCM (10 mL) was added TFA (1 mL) and stirred at 25° C. for 2 h under N₂. The mixture was concentrated under vacuum. LC-MS: m/z 234.2 (M+H)⁺. Then MeOH (20 mL) and Pd/C 10% (63.85 mg, 0.600 mmol) were added. The reaction mixture was stirred at 25° C. for 18 hours under H₂. The mixture was filtered and concentrated under vacuum to afford 1,1,1-trifluoro-3-methoxypropan-2-amine (85 mg, 99.00%) as a yellow oil, which was used in the next step without further purification. LC-MS: m/z 144.2 (M+H)⁺.

### Step E: 1-(1,1,1-trifluoro-3-methoxypropan-2-yl)-1H-imidazol-5-amine

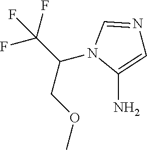

To a solution of {[(E)-methoxymethylidene]amino}acetonitrile (118 mg, 1.203 mmol) in DCM (10 mL) was added 1,1,1-trifluoro-3-methoxypropan-2-amine (85 mg, 0.594 mmol), the mixture was stirred at 40° C. for 2 h under N₂. The reaction mixture was concentrated under vacuum and the resulting residue was purified by prep-HPLC (A: 0.1% FA/H₂O, B: ACN; Flow rate: 45 ml/min; Gradient: 0%-5%) to afford 1-(1,1,1-trifluoro-3-methoxypropan-2-yl)-1H-imidazol-5-amine (70 mg, 56.34%) as a yellow oil. LC-MS: m/z 210.2 (M+H)⁺.

The following compounds were synthesized following the similar route of Example I6 with appropriate starting materials.

| Name | Structure | LC-MS/NMR |
|---|---|---|
| (S)-1-(1-(4-fluorophenyl)ethyl)-1H- imidazol-5-amine | 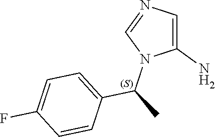 | LC-MS: m/z 206.1 (M + H)⁺ ¹H NMR (400 MHZ, DMSO- d₆) δ 7.34-7.30 (m, 1H), 7.27- 7.21 (m, 2H), 7.19 - 7.13 (m, 2H), 6.14 - 6.00 (m, 1H), 5.39- 5.29 (m, 1H), 4.38 - 4.19 (m, 2H), 1.72 (d, J = 7.2 Hz, 3H) |
| 1-(1-(4-fluorophenyl)cyclopropyl)-1H- imidazol-5-amine | 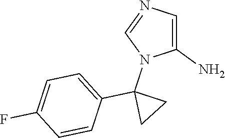 [SMILES: Nc1cncn1C1(c2ccc(F)cc2)CC1] | LC-MS: m/z 218.1 (M + H)⁺ |
| 1-(2-(4-fluorophenyl)propan-2-yl)-1H- imidazol-5-amine | 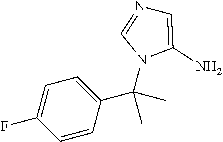 [SMILES: CC(C)(c1ccc(F)cc1)n1cncc1N] | LC-MS: m/z 220.2 (M + H)⁺ |
| (S)-1-(1-(5-fluoropyridin-2-yl)ethyl)- 1H-imidazol-5-amine | 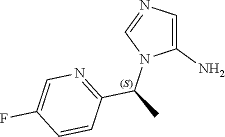 | LC-MS: m/z 207.2 (M + H)⁺ |
| 1-(1-(5-fluoropyridin-2-yl)cyclopropyl)- 1H-imidazol-5-amine | 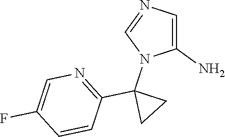 [SMILES: Nc1cncn1C1(c2ccc(F)cn2)CC1] | LC-MS: m/z 219.1 (M + H)⁺ |
| 1-(1-(5-fluoropyrimidin-2-yl)ethyl)-1H- imidazol-5-amine | 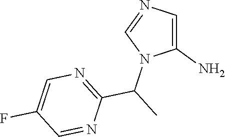 | LC-MS: m/z 208.1 (M + H)⁺ |
| 1-(2,2,2-trifluoroethyl)-1H-imidazol-5- amine | 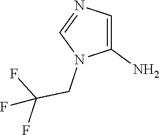 [SMILES: Nc1cncn1CC(F)(F)F] | LC-MS: m/z 166.1 (M + H)⁺ |
| (S)-1-(1-(trifluoromethoxy)propan-2- yl)-1H-imidazol-5-amine | 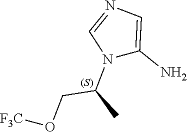 | LC-MS: m/z 210.1 (M + H)⁺ |
| methyl (S)-4-(1-(5-amino-1H-imidazol- 1-yl)ethyl)piperidine-1-carboxylate | 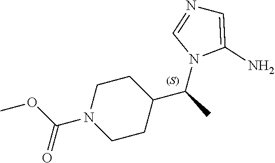 | LC-MS: m/z 253.2 (M + H)⁺ |
| 1-(1-(trifluoromethyl)cyclopropyl)-1H- imidazol-5-amine | 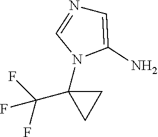 [SMILES: Nc1cncn1C1(C(F)(F)F)CC1] | LC-MS: m/z 192.1 (M + H)⁺ |
| 1-(1-(difluoromethyl)cyclopropyl)-1H- imidazol-5-amine | 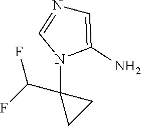 [SMILES: Nc1cncn1C1(C(F)F)CC1] | LC-MS: m/z 174.1 (M + H)⁺ |
| 1-(1-(methoxymethyl)cyclopropyl)-1H- imidazol-5-amine | 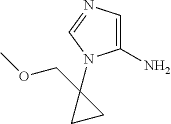 | LC-MS: m/z 168.1 (M + H)⁺ |
| 1-(1-((trifluoromethoxy)methyl) cyclopropyl)-1H-imidazol-5-amine | 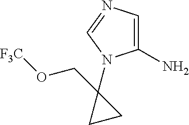 [SMILES: Nc1cncn1C1(COC(F)(F)F)CC1] | LC-MS: m/z 222.1 (M + H)⁺ |
| 1-(1-((difluoromethoxy)methyl) cyclopropyl)-1H-imidazol-5-amine | 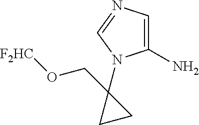 [SMILES: Nc1cncn1C1(COC(F)F)CC1] | LC-MS: m/z 204.1 (M + H)⁺ |
| 2-methyl-1-(1- (trifluoromethyl)cyclopropyl)-1H- imidazol-5-amine | 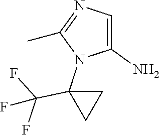 | LC-MS: m/z 206.1 (M + H)⁺ |

### Example I7: 2-isopropyl-1-(1-(trifluoromethyl)cyclopropyl)-1H-imidazol-5-amine

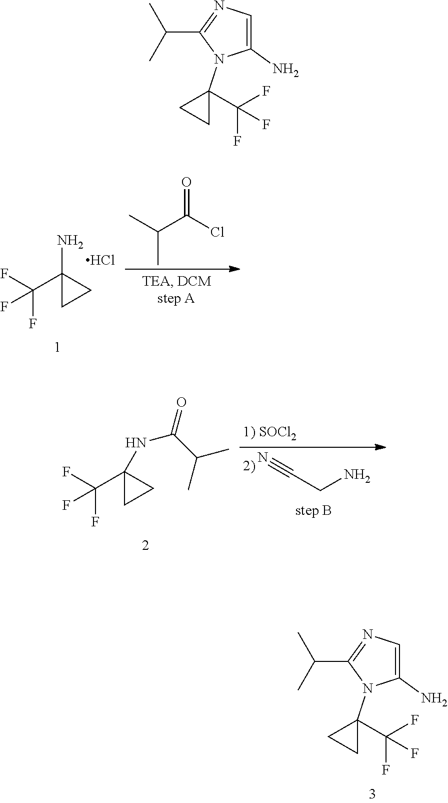

### Step A: N-(1-(trifluoromethyl)cyclopropyl)isobutyramide

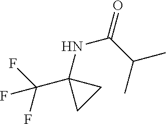

To a solution of 1-(trifluoromethyl)cyclopropanamine (1.0 g, 6.19 mmol, HCl salt) in DCM (10 mL) was added TEA (1.88 g, 18.57 mmol, 2.58 mL) and 2-methylpropanoyl chloride (726 mg, 6.81 mmol, 713.86 μL). The mixture was stirred at 25° C. for 1 hr. The reaction mixture was quenched by addition of H₂O (30 mL) at 20° C., and then extracted with EtOAc (30 mL×2). The combined organic layers were washed with brine (30 mL), dried over Na₂SO₄, filtered and concentrated under reduced pressure to give a residue. The residue was purified by flash silica gel chromatography (ISCO®; 20 g SepaFlash® Silica Flash Column, Eluent of 0~10% Ethyl acetate/Petroleum ether gradient @ 60 mL/min) to afford N-(1-(trifluoromethyl)cyclopropyl)isobutyramide (1.1 g, 91.05%) as a white solid. ¹H NMR (400 MHz, CDCl₃) § 5.85 (br s, 1H), 2.39-2.27 (m, 1H), 1.35-1.29 (m, 2H), 1.15 (d, J=6.8 Hz, 7H), 1.13-1.07 (m, 2H). LC-MS: m/z 195.9 (M+H)⁺.

### Step B: 2-isopropyl-1-(1-(trifluoromethyl)cyclopropyl)-1H-imidazol-5-amine

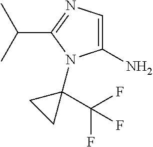 [SMILES: CC(C)c1ncc(N)n1C1(C(F)(F)F)CC1]

A mixture of N-(1-(trifluoromethyl)cyclopropyl)isobutyramide (0.9 g, 4.61 mmol), SOCl₂ (36.85 g, 309.78 mmol, 22.50 mL) was stirred at 100° C. for 4 hr under N₂ atmosphere. And the reaction mixture was concentrated in vacuo and to the residue was added dioxane (5 mL), the mixture was concentrated in vacuo to remove the trace SOCl₂. The residue was solved with dioxane (10 mL) and to the mixture was added 2-aminoacetonitrile (1.35 g, 24.08 mmol) in dioxane (1 mL) at 25° C. A lot of white solid was observed. After the mixture was stirred at 25° C. for 16 hr. The reaction mixture was concentrated in vacuo. The residue was purified by flash silica gel chromatography (ISCO®; 12 g Sepa Flash® Silica Flash Column, Eluent of 0~5% MeOH/DCM gradient @50 mL/min) to afford 2-isopropyl-1-(1-(trifluoromethyl)cyclopropyl)-1H-imidazol-5-amine (190 mg, 17.67%) as a brown solid. LC-MS: m/z 234.1 (M+H)⁺.

### Example I8: 2-(5-amino-1H-pyrazol-1-yl)-2-(5-fluoropyridin-2-yl) ethan-1-ol and 1-(2-((tert-butyldimethylsilyl)oxy)-1-(5-fluoropyridin-2-yl)ethyl)-3-chloro-1H-pyrazol-5-amine

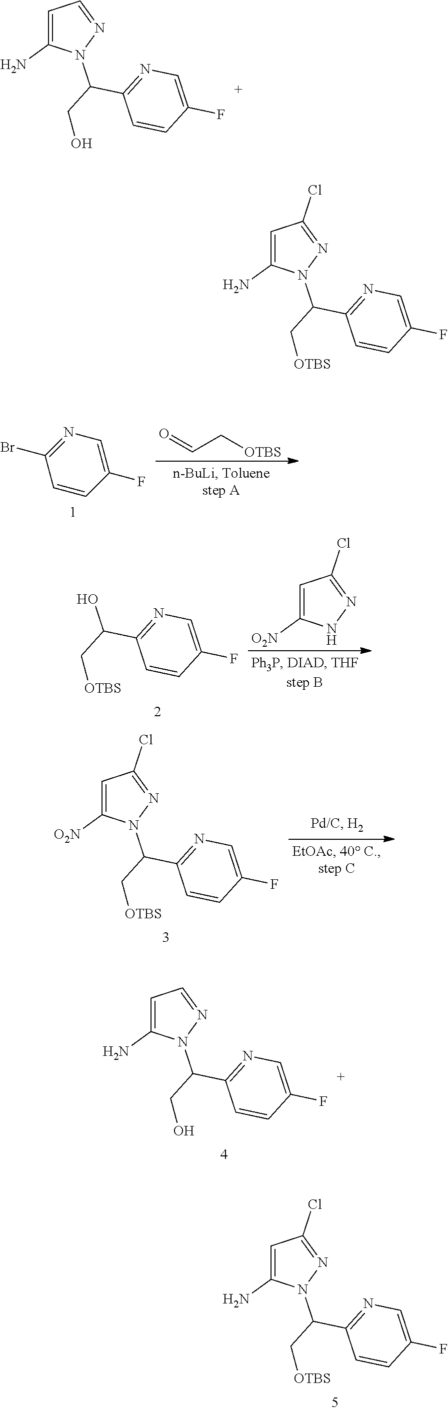

### Step A: 2-((tert-butyldimethylsilyl)oxy)-1-(5-fluoropyridin-2-yl) ethan-1-ol

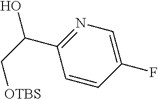 [SMILES: CC(C)(C)[Si](C)(C)OCC(O)c1ccc(F)cn1]

To a solution of butyllithium (2.5 M, 11.36 mL) in toluene (100.0 mL) was added a solution of 2-bromo-5-fluoropyridine (5 g, 28.41 mmol) in toluene (60.0 mL) at −78° C. under N₂ and the mixture was stirred at −78° C. for 30 min. Then 2-((tert-butyldimethylsilyl)oxy) acetaldehyde (4.46 g, 25.57 mmol, 4.87 mL) was added at −78° C. under N₂ and the mixture was stirred at −78° C. for 2 hours under N₂ atmosphere. The mixture was quenched by the addition of aqueous NH₄Cl (3 mL) at 0° C. The mixture was diluted with H₂O (2 mL) and extracted with ethyl acetate (4 mL×3). The combined organic layers were washed with brine (2 mL), dried over Na₂SO₄, filtered and concentrated under reduced pressure to give a crude. The crude was purified by flash silica gel chromatography (ISCOR; 80 g SepaFlash® Silica Flash Column, Eluent of 0~50% Ethyl acetate/Petroleum ether gradient @ 25 mL/min) and concentrated under reduced pressure to afford 2-((tert-butyldimethylsilyl)oxy)-1-(5-fluoropyridin-2-yl) ethan-1-ol (3.98 g, 51.62%) as a yellow oil.

¹H NMR (400 MHz, CDCl₃) δ 8.40 (d, J=2.8 Hz, 1H), 7.51-7.46 (m, 1H), 7.46-7.37 (m, 1H), 4.79 (t, J=5.6 Hz, 1H), 3.88-3.82 (m, 1H), 3.82-3.76 (m, 1H), 0.86 (s, 9H), 0.04-−0.02 (m, 6H).

### Step B: 2-(2-((tert-butyldimethylsilyl)oxy)-1-(3-chloro-5-nitro-1H-pyrazol-1-yl)ethyl)-5-fluoropyridine

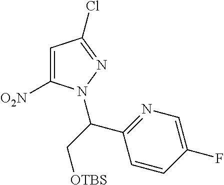 [SMILES: CC(C)(C)[Si](C)(C)OCC(c1ccc(F)cn1)n1nc(Cl)cc1[N+](=O)[O-]]

To a solution of 2-((tert-butyldimethylsilyl)oxy)-1-(5-fluoropyridin-2-yl) ethan-1-ol (1.84 g, 6.78 mmol), 3-chloro-5-nitro-1H-pyrazole (1.0 g, 6.78 mmol) and PPh₃ (1.33 g, 5.08 mmol) in THF (10.0 mL) was added DIAD (1.03 g, 5.08 mmol, 985.66 μL, 0.75 eq) at 0° C. under N₂ and the mixture was stirred at 25° C. for 18 hours under N₂. The volatiles were removed in vacuo to give a crude. The crude was purified by flash silica gel chromatography (ISCOR; 40 g SepaFlash® Silica Flash Column, Eluent of 0~3% Ethyl acetate/Petroleum ether gradient @ 25 mL/min) to afford 2-(2-((tort-butyldimethylsilyl)oxy)-1-(3-chloro-5-nitro-1H-pyrazol-1-yl)ethyl)-5-fluoropyridine (1.33 g, 47.78%) as a yellow oil. LC-MS: m/z 401.1 (M+H)⁺. ¹H NMR (400 MHz, CDCl₃) δ 8.44 (d, J=2.8 Hz, 1H), 7.46-7.35 (m, 2H), 6.99 (s, 1H), 6.55-6.47 (m, 1H), 4.51-4.43 (m, 1H), 4.36-4.29 (m, 1H), 0.79 (s, 9H), 0.05-−0.06 (m, 6H).

### Step C: 2-(5-amino-1H-pyrazol-1-yl)-2-(5-fluoropyridin-2-yl) ethan-1-ol and 1-(2-((tert-butyldimethylsilyl)oxy)-1-(5-fluoropyridin-2-yl)ethyl)-3-chloro-1H-pyrazol-5-amine

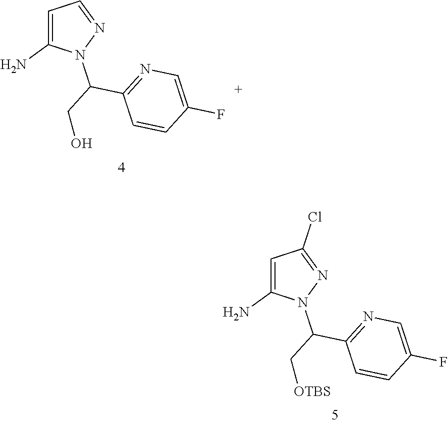 [SMILES: Nc1ccnn1C(CO)c1ccc(F)cn1 ; CC(C)(C)[Si](C)(C)OCC(c1ccc(F)cn1)n1nc(Cl)cc1N]

To a solution of 2-(2-((tert-butyldimethylsilyl)oxy)-1-(3-chloro-5-nitro-1H-pyrazol-1-yl)ethyl)-5-fluoropyridine (650.0 mg, 1.62 mmol) in ethyl acetate (10.0 mL) was added Pd/C (172.54 mg, 162.13 μmol, 10% purity) under N₂ atmosphere. The suspension was degassed and purged with H₂ for 3 times. The mixture was stirred under H₂ (15 Psi) at 40° C. for 16 hour. The reaction mixture was filtered and the filtrate was concentrated under reduced pressure to give a residue. The crude was purified by flash silica gel chromatography (ISCOR; 12 g SepaFlash® Silica Flash Column, Eluent of 0~20% Dichloromethane:Methanol @ 15 mL/min) to afford 2-(5-amino-1H-pyrazol-1-yl)-2-(5-fluoropyridin-2-yl) ethan-1-ol (40.0 mg, 10.87%) as a colorless oil and 1-(2-((tert-butyldimethylsilyl)oxy)-1-(5-fluoropyridin-2-yl)ethyl)-3-chloro-1H-pyrazol-5-amine (230.0 mg, 32.14%) as a colorless oil.

Compound 4: ¹H NMR (400 MHz, CDCl₃) δ 8.32 (d, J=2.4 Hz, 1H), 7.30 (dd, J=8.4, 2.8 Hz, 1H), 7.27-7.24 (m, 1H), 7.07-6.99 (m, 1H), 5.49 (d, J=2.0 Hz, 1H), 5.28 (t, J=4.8 Hz, 1H), 4.33 (d, J=4.8 Hz, 2H). LC-MS: m/z 223.1 (M+H)⁺.

Compound 5: ¹H NMR (400 MHz, CDCl₃) δ 8.41 (d, J=2.4 Hz, 1H), 7.43-7.33 (m, 2H), 5.49 (s, 1H), 5.45 (t, J=7.2 Hz, 1H), 4.49-4.36 (m, 2H), 4.25 (s, 2H), 0.80 (s, 9H), 0.02-−0.04 (m, 6H). LC-MS: m/z 371.1 (M+H)⁺.

### 3-chloro-1-((5,6,7,8-tetrahydro-[1,2,4]triazolo[1,5-a]pyridin-7-yl)methyl)-1H-pyrazol-5-amine

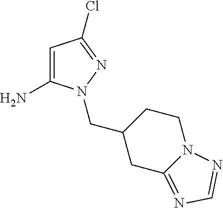 [SMILES: Nc1cc(Cl)nn1CC1CCn2ncnc2C1]

3-chloro-1-((5,6,7,8-tetrahydro-[1,2,4]triazolo[1,5-a]pyridin-7-yl)methyl)-1H-pyrazol-5-amine was synthesized following the similar route of Example 18, using (5,6,7,8-tetrahydro-[1,2,4]triazolo[1,5-a]pyridin-7-yl) methanol in step B. LC-MS: m/z 253.1 (M+H)⁺.

### Example I9: 4-cyclopropyl-3-oxobutanamide

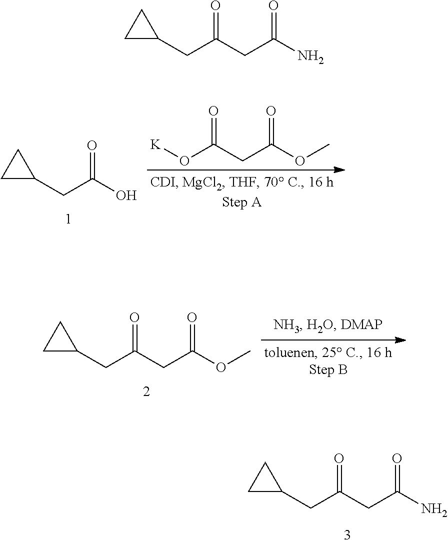

### Step A: methyl 4-cyclopropyl-3-oxobutanoate

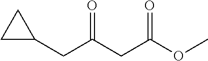

To a solution of 2-cyclopropylacetic acid (2000 mg, 19.976 mmol) in THF (80 mL) was added CDI (6478.23 mg, 39.952 mmol), the reaction mixture was stirred at 25° C. for 2 hrs under N₂. Potassium 3-methoxy-3-oxopropanoate (6239.71 mg, 39.952 mmol) was added, the mixture was stirred at 25° C. for 30 min, magnesium chloride (3803.44 mg, 39.952 mmol) was added. The reaction mixture was stirred at 70° C. for another 16 hrs under N₂. Water (100 mL) was added to quench the reaction, the mixture was extracted with EA (80 mL), the organic layer was separated and washed with brine (40 mL). The organic layer was dried over sodium sulfate, filtered and concentrated under vacuum. The resulting residue was purified by silica gel column chromatography (eluting PE/EA=8/1) to afford methyl 4-cyclopropyl-3-oxobutanoate (1160 mg, 37.18%) as a yellow oil. LC-MS: m/z 157.0 (M+H)⁺.

### Step B: 4-cyclopropyl-3-oxobutanamide

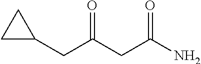

To a solution of methyl 4-cyclopropyl-3-oxobutanoate (1160 mg, 7.427 mmol) in toluene (10 mL) were added DMAP (907.40 mg, 7.427 mmol) and ammonium hydroxide (5.721 mL, 37.135 mmol), the reaction mixture was stirred at 25° C. for 18 hrs under N₂. The mixture was concentrated under vacuum. The resulting residue was purified by silica gel chromatography (eluting DCM/CH₃OH=100/1) to afford 4-cyclopropyl-3-oxobutanamide (300 mg, 28.61%) as a white solid. ¹H NMR (400 MHz, DMSO-d₆) δ 7.37 (s, 1H), 6.94 (s, 1H), 3.02 (s, 2H), 2.31 (d, J=6.8 Hz, 2H), 0.80-0.84 (m, 1H), 0.36-0.40 (m, 2H), 0.00-0.04 (m, 2H). LC-MS: m/z 142.0 (M+H)⁺.

### 4,5-dimethyl-3-oxohexanamide

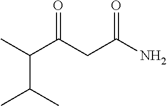

4,5-dimethyl-3-oxohexanamide was synthesized following the similar route of Example I9, using 2,3-dimethylbutanoic acid in step A. LC-MS: m/z 158.1 (M+H)⁺.

### Example I10: 4-(1-((4-methoxybenzyl)oxy)cyclopropyl)-3-oxobutanamide

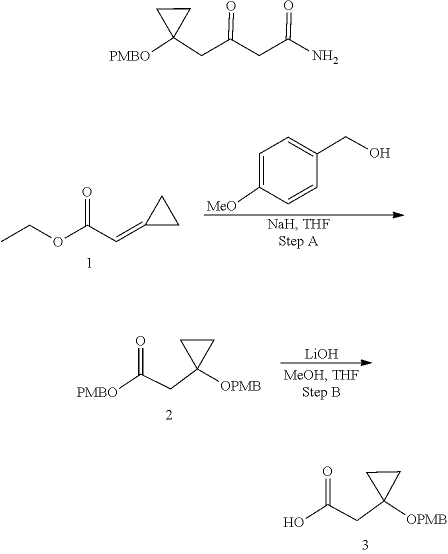

### Step A: 4-methoxybenzyl 2-(1-((4-methoxybenzyl)oxy)cyclopropyl)acetate

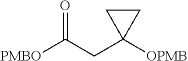

To a solution of NaH (159 mg, 3.98 mmol, 60% purity) in THF (5 mL) was added (4-methoxyphenyl) methanol (8.20 g, 59.34 mmol, 7.4 mL). The mixture was stirred for 30 minutes, before ethyl 2-cyclopropylideneacetate (2.5 g, 19.82 mmol) in THF (2.5 mL) was added. The reaction was stirred at 15-20° C. for 2 hours. H₂O (2 mL) was added. The organic layers were dried over anhydrous sodium sulfate, filtered and concentrated to give the crude product. The crude product was purified by column chromatography (SiO₂, 0~30% ethyl acetate in petroleum ether) to give 4-methoxybenzyl 2-(1-((4-methoxybenzyl)oxy)cyclopropyl)acetate (3.0 g, 42.47% yield) as colorless oil.

¹H NMR (400 MHz, CDCl₃) δ 7.33-7.28 (m, 2H), 7.20-7.11 (m, 2H), 6.90-6.84 (m, 2H), 6.84-6.78 (m, 2H), 5.11 (s, 2H), 4.45 (s, 2H), 3.80 (d, J=7.2 Hz, 6H), 2.69 (s, 2H), 1.02-0.92 (m, 2H), 0.72-0.60 (m, 2H).

### Step B: 2-(1-((4-methoxybenzyl)oxy)cyclopropyl)acetic acid

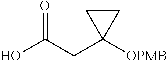

To a solution of 4-methoxybenzyl 2-(1-((4-methoxybenzyl)oxy)cyclopropyl)acetate (2.0 g, 5.61 mmol) in THF (10 mL) and MeOH (10 mL) was added LiOH H₂O (2 M, 10 mL). The mixture was stirred at 15-20° C. for 2 hours. The reaction was concentrated and diluted with water (30 mL), extracted with ethyl acetate (50 mL×2). The aqueous layer was acidified by HCl (2M) to pH=2-3, extracted with ethyl acetate (60 mL×3). The organic layers were dried over anhydrous Na₂SO₄, filtered and concentrated to give crude 2-(1-((4-methoxybenzyl)oxy)cyclopropyl)acetic acid (1.3 g, 98.05% yield) as a colorless oil.

¹H NMR (400 MHz, CDCl₃) δ 7.26-7.20 (m, 2H), 6.90-6.83 (m, 2H), 4.52 (s, 2H), 3.80 (s, 3H), 2.69 (s, 2H), 1.08-1.00 (m, 2H), 0.76-0.65 (m, 2H).

### 4-(1-((4-methoxybenzyl)oxy)cyclopropyl)-3-oxobutanamide

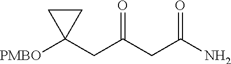

4-(1-((4-methoxybenzyl)oxy)cyclopropyl)-3-oxobutanamide was then synthesized following the similar route of Example 19, using 2-(1-((4-methoxybenzyl)oxy)cyclopropyl)acetic acid in step A. LC-MS: m/z 278.1 (M−H)⁺.

### Example I11: tert-butyl(4-cyclopropyl-3-oxobutanoyl)carbamate

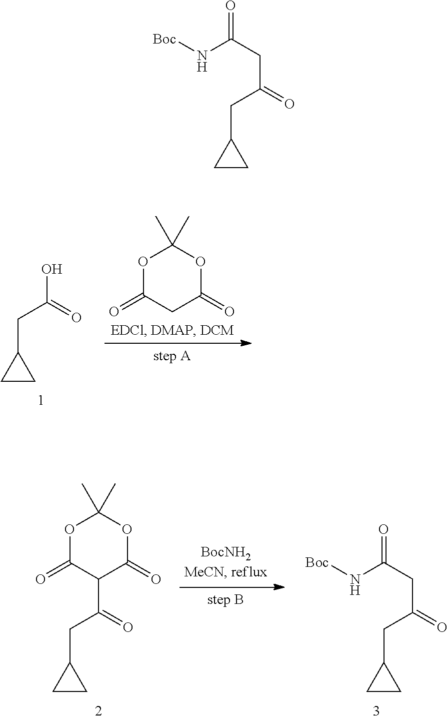

### Step A: 5-(2-cyclopropylacetyl)-2,2-dimethyl-1,3-dioxane-4,6-dione

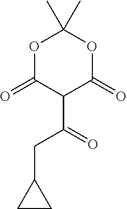 [SMILES: CC1(C)OC(=O)C(C(=O)CC2CC2)C(=O)O1]

To a solution of 2,2-dimethyl-1,3-dioxane-4,6-dione (7.92 g, 54.94 mmol), 2-cyclopropylacetic acid (5 g, 49.94 mmol), DMAP (8.54 g, 69.92 mmol) in DCM (100 mL) was added EDCl (13.40 g, 69.92 mmol). The mixture was stirred at 25° C. for 16 hours. The mixture was washed with HCl (0.5 N, 20 mL*2), brine (20 mL), dried over Na₂SO₄, filtered and concentrated to give a crude 5-(2-cyclopropylacetyl)-2,2-dimethyl-1,3-dioxane-4,6-dione (11 g, 97.36% yield) as a yellow oil, which was used in the next step without further purification. ¹H NMR (400 MHz, CDCl₃) δ 6.68 (d, J=7.6 Hz, 1H), 2.91 (d, J=7.2 Hz, 2H), 1.69 (s, 6H), 1.07-1.19 (m, 1H), 0.42-0.54 (m, 2H), 0.20-0.24 (m, 2H). LC-MS: m/z 227.0 (M+H)⁺.

### Step B: tert-butyl(4-cyclopropyl-3-oxobutanoyl)carbamate

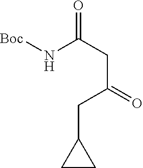 [SMILES: CC(C)(C)OC(=O)NC(=O)CC(=O)CC1CC1]

To a solution of 5-(2-cyclopropylacetyl)-2,2-dimethyl-1,3-dioxane-4,6-dione (11 g, 48.62 mmol) in MeCN (100 mL) was added tert-butyl carbamate (6.84 g, 58.35 mmol). The mixture was stirred at 84° C. for 2 hr. The reaction mixture concentrated under reduced pressure to give a residue, which was purified by flash silica gel chromatography (ISCO®; 40 g SepaFlash® Silica Flash Column, Eluent of 0~100% Ethyl acetate/Petroleum other gradient @ 50 mL/min) to give tort-butyl(4-cyclopropyl-3-oxobutanoyl) carbamate (5.37 g, 45.77%) as a yellow solid. ¹H NMR (400 MHz, DMSO-d₆) δ 10.38 (s, 1H), 3.70 (s, 1H), 2.40 (d, J=6.8 Hz, 2H), 1.36 (s, 9H), 0.84-0.95 (m, 1H), 0.42-0.52 (m, 2H), 0.04-0.09 (m, 2H). LC-MS: m/z 186.0 (M+H−56)⁺.

### tert-butyl(5,5,5-trifluoro-3-oxopentanoyl)carbamate

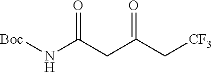 [SMILES: CC(C)(C)OC(=O)NC(=O)CC(=O)CC(F)(F)F]

tert-butyl(5,5,5-trifluoro-3-oxopentanoyl)carbamate was synthesized following the similar route of Example I11, using 3,3,3-trifluoropropanoic acid in step A. LC-MS: m/z 270.1 (M+H)⁺.

### tert-butyl(6,6,6-trifluoro-5-methyl-3-oxohexanoyl)carbamate

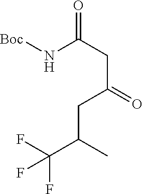 [SMILES: CC(CC(=O)CC(=O)NC(=O)OC(C)(C)C)C(F)(F)F]

tert-butyl(5,5,5-trifluoro-3-oxopentanoyl)carbamate was synthesized following the similar route of Example I11, using 4,4,4-trifluoro-3-methylbutanoic acid in step A. LC-MS: m/z 298.2 (M+H)⁺.

### Example I2: 3-((5-fluoropyridin-2-yl)methyl)benzo[d]isoxazole-6-carbaldehyde

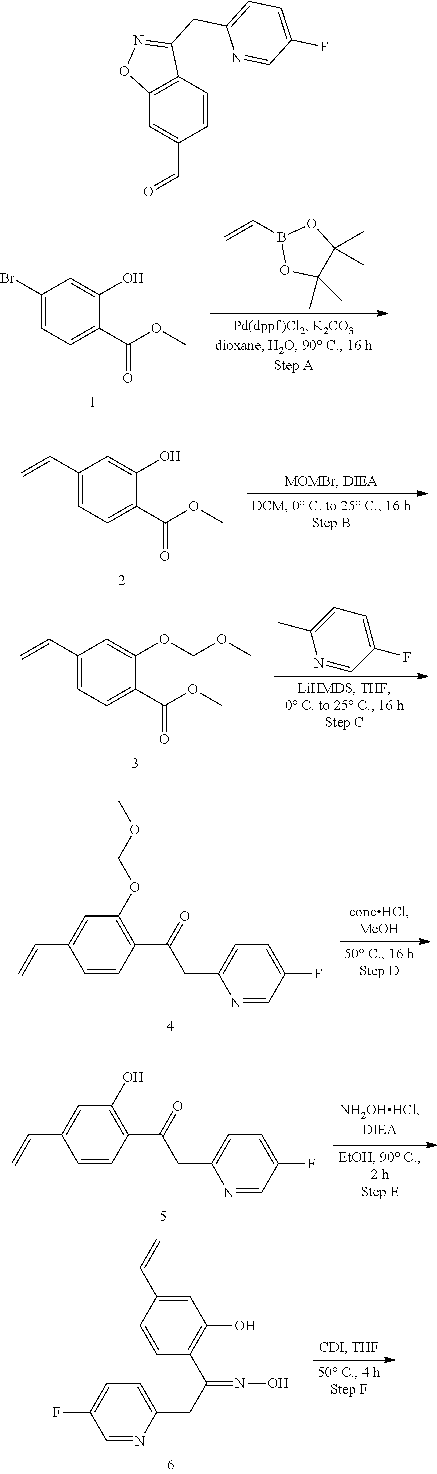

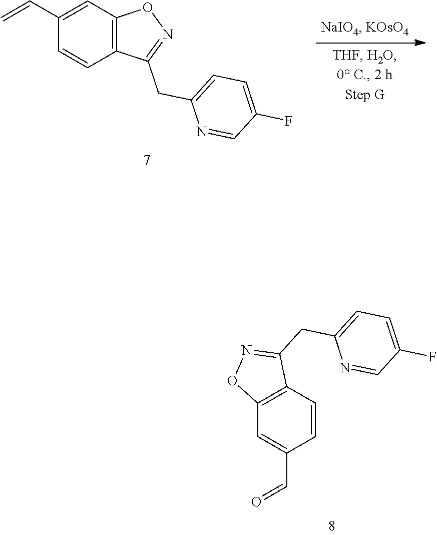 [SMILES: C=Cc1ccc2c(Cc3ccc(F)cn3)noc2c1 ; O=Cc1ccc2c(Cc3ccc(F)cn3)noc2c1]

### Step A: methyl 2-hydroxy-4-vinylbenzoate

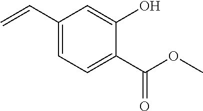

To a solution of methyl 4-bromo-2-hydroxybenzoate (18.64 g, 80.679 mmol) in dioxane (400 mL) and H₂O (40 mL) were added 4,4,5,5-tetramethyl-2-vinyl-1,3,2-dioxaborolane (24.85 g, 161.357 mmol), K₂CO₃ (33.45 g, 242.036 mmol) and Pd(dppf)Cl₂ (5.90 g, 8.068 mmol). The reaction mixture was stirred at 90° C. for 16 hrs under N₂. The mixture was concentrated under vacuum. The resulting residue was purified by silica gel chromatography (PE/EA=10/1) to afford methyl 2-hydroxy-4-vinylbenzoate (13.8 g, 95.99%) as a colorless oil. ¹H NMR (400 MHz, CDCl₃) δ 10.66 (s, 1H), 7.70 (d, J=8.4 Hz, 1H), 6.92 (d, J=1.6 Hz, 1H), 6.86 (dd, J=8.0, 1.2 Hz, 1H), 6.59 (dd, J=17.6, 10.8 Hz, 1H), 5.78 (dd, J=17.6, 0.4 Hz, 1H), 5.32 (d, J=10.8 Hz, 1H), 3.86 (s, 3H). LC-MS: m/z 179.3 (M+H)⁺.

### Step B: methyl 2-[(methoxymethyl)oxy]-4-vinylbenzoate

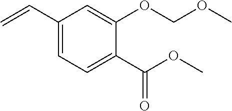 [SMILES: C=Cc1ccc(C(=O)OC)c(OCOC)c1]

To a solution of methyl 2-hydroxy-4-vinylbenzoate (14 g, 78.568 mmol) in DCM (300 mL) were added DIEA (15.23 g, 117.852 mmol) and bromo(methoxy)methane (14.73 g, 117.852 mmol) at 0° C. under N₂, The reaction mixture was stirred at 25° C. for 16 hrs under N₂. The mixture was concentrated under vacuum. To the resulting residue MTBE (100 mL) and EA (50 mL)/PE (50 mL) was added, the white solid was filtered and the filtrate was concentrated under vacuum to afford methyl 2-[(methoxymethyl)oxy]-4-vinylbenzoate (16.6 g, 95.07%) as a yellow oil, which was used in the next step without further purification. LC-MS: m/z 245.2 (M+Na)⁺.

### Step C: 2-(5-fluoropyridin-2-yl)-1-{2-[(methoxymethyl)oxy]-4-vinylphenyl}ethan-1-one

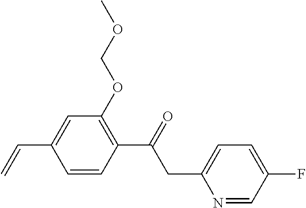 [SMILES: C=Cc1ccc(C(=O)Cc2ccc(F)cn2)c(OCOC)c1]

To a solution of methyl 2-[(methoxymethyl)oxy]-4-vinylbenzoate (15.40 g, 69.294 mmol) and 5-fluoro-2-methylpyridine (7 g, 62.995 mmol) in THF (300 mL) was added 2-(methoxymethyl) oxiran-1-ium (188.985 mL, 188.985 mmol) at 0° C. under N₂. The reaction mixture was stirred at 25° C. for 16 hrs under N₂. The mixture was added to ice-water (500 mL), extracted with EA (300 mL×3), the combined organic layers were washed with brine (200 mL), dried over sodium sulfate, filtered and concentrated in vacuum. The resulting residue was purified by silica gel chromatography (eluting PE/EA=10/1) to afford 2-(5-fluoropyridin-2-yl)-1-{2-[(methoxymethyl)oxy]-4-vinylphenyl}ethan-1-one (16.12 g, 84.92%) as a yellow oil. ¹H NMR (400 MHz, DMSO-d₆) δ 8.45 (d, J=3.2 Hz, 1H), 7.61-7.73 (m, 2H), 7.40-7.43 (m, 1H), 7.28 (d, J=0.8 Hz, 1H), 7.21 (dd, J=8.4, 0.8 Hz, 1H), 6.72-6.79 (m, 1H), 5.96 (d, J=17.6 Hz, 1H), 5.42 (d, J=11.2 Hz, 1H), 5.37 (s, 2H), 4.50 (s, 2H), 3.42 (s, 3H). LC-MS: m/z 302.3 (M+H)⁺.

### Step D: 2-(5-fluoropyridin-2-yl)-1-(2-hydroxy-4-vinylphenyl) ethan-1-one

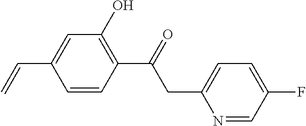 [SMILES: C=Cc1ccc(C(=O)Cc2ccc(F)cn2)c(O)c1]

To a solution of 2-(5-fluoropyridin-2-yl)-1-{2-[(methoxymethyl)oxy]-4-vinylphenyl}ethan-1-one (12 g, 39.825 mmol) in MeOH (200 mL) was added HCl (33.187 mL, 398.248 mmol), The reaction mixture was stirred at 50° C. for 16 hrs. The mixture was concentrated in vacuum to afford 2-(5-fluoropyridin-2-yl)-1-(2-hydroxy-4-vinylphenyl) ethan-1-one (11.7 g, 100.0%) as a yellow solid, which was used in the next step without further purification. LC-MS: m/z 258.2 (M+H)⁺.

### Step E: 2-(5-fluoropyridin-2-yl)-1-(2-hydroxy-4-vinylphenyl) ethan-1-one oxime

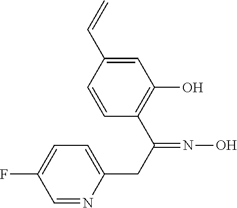 [SMILES: C=Cc1ccc(C(Cc2ccc(F)cn2)=NO)c(O)c1]

To a solution of 2-(5-fluoropyridin-2-yl)-1-(2-hydroxy-4-vinylphenyl) ethan-1-one (6 g, 23.323 mmol) in EtOH (150 mL) were added azanol hydrochloride (8.10 g, 116.614 mmol) and DIEA (20.313 mL, 116.614 mmol), The reaction mixture was stirred at 90° C. for 2 hrs under N₂. The mixture was added to EA (50 mL), washed with brine (20 mL), dried over sodium sulfate, filtered and concentrated in vacuum. The resulting residue was purified by silica gel chromatography (eluting PE/EA=3/1) to afford 2-(5-fluoropyridin-2-yl)-1-(2-hydroxy-4-vinylphenyl) ethan-1-one oxime (5.5 g, 86.61%) as a white solid. ¹H NMR (400 MHz, DMSO-d₆) δ 11.81 (s, 1H), 11.52 (s, 1H), 8.43 (d, J=2.8 Hz, 1H), 7.62-7.68 (m, 1H), 7.47 (d, J=8.0 Hz, 1H), 7.37 (dd, J=8.8, 4.4 Hz, 1H), 6.93-6.96 (m, 2H), 6.64 (dd, J=19.2, 10.8 Hz, 1H), 5.84 (d, J=18.0 Hz, 1H), 5.28 (d, J=11.6 Hz, 1H), 4.37 (s, 2H). LC-MS: m/z 273.2 (M+H)⁺.

### Step F: 3-((5-fluoropyridin-2-yl)methyl)-6-vinylbenzo[d]isoxazole

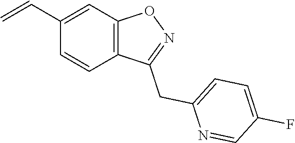 [SMILES: C=Cc1ccc2c(Cc3ccc(F)cn3)noc2c1]

To a solution of 2-(5-fluoropyridin-2-yl)-1-(2-hydroxy-4-vinylphenyl) ethan-1-one oxime (6 g, 22.036 mmol) in THF (500 mL) was added CDI (7.15 g, 44.072 mmol), the mixture was stirred at 50° C. for 4 hrs under N₂. The mixture was concentrated under vacuum. The resulting residue was purified by silica gel chromatography (eluting PE/EA=9/1) to afford 3-((5-fluoropyridin-2-yl)methyl)-6-vinylbenzo[d]isoxazole (4.2 g, 74.96%) as a yellow solid. ¹H NMR (400 MHz, DMSO-d₆) δ 8.48 (d, J=2.8 Hz, 1H), 7.79 (s, 1H), 7.70-7.75 (m, 1H), 7.58-7.63 (m, 2H), 7.49 (d, J=8 Hz, 1H), 6.88 (dd, J=17.6, 11.2 Hz, 1H), 6.04 (d, J=17.6 Hz, 1H), 5.43 (d, J=11.2 Hz, 1H), 4.54 (s, 2H). LC-MS: m/z 255.2 (M+H)⁺.

### Step G: 3-((5-fluoropyridin-2-yl)methyl)benzo[d]isoxazole-6-carbaldehyde

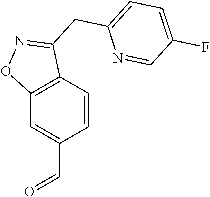 [SMILES: O=Cc1ccc2c(Cc3ccc(F)cn3)noc2c1]

To a solution of 3-((5-fluoropyridin-2-yl)methyl)-6-vinylbenzo[d]isoxazole (500 mg, 1.966 mmol) in THF (10 mL) were added a solution of NaIO₄ (1682.45 mg, 7.866 mmol) in H₂O (10 mL), followed by dipotassium dioxidodioxo-λ6-osmium^{(VI)} dihydrate (72.46 mg, 0.197 mmol), The reaction mixture was stirred at 0° C. for 2 hrs under N₂. The mixture was filtered, the filtrate was purified by C18 (eluting 44% of ACN in water, 0.1% FA) to afford 3-((5-fluoropyridin-2-yl)methyl)benzo[d]isoxazole-6-carbaldehyde (353 mg, 70.05%) as a white solid. ¹H NMR (400 MHz, DMSO-d₆) δ 10.15 (s, 1H), 8.47 (d, J=3.2 Hz, 1H), 8.29 (d, J=1.2 Hz, 1H), 7.84-7.90 (m, 2H), 7.72-7.77 (m, 1H), 7.63 (dd, J=8.4, 4.4 Hz, 1H), 4.63 (s, 2H). LC-MS: m/z 257.2 (M+H)⁺.

### 3-((5-chloropyridin-2-yl)methyl)benzo[d]isoxazole-6-carbaldehyde

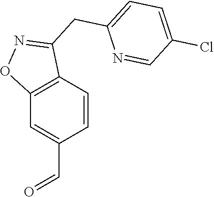 [SMILES: O=Cc1ccc2c(Cc3ccc(Cl)cn3)noc2c1]

3-((5-chloropyridin-2-yl)methyl)benzo[d]isoxazole-6-carbaldehyde was synthesized following the similar route of Example I12, using 5-chloro-2-methylpyridine in step C. LC-MS: m/z 273.2 (M+H)⁺.

### Example 1

### (S)-7-{7-[(R)-4-aza-1-indanylamino]-1-thia-6-aza-2-indenyl}-3-[(p-fluorophenyl)methyl]-5-isobutyl-3-methyl-1-oxo-2,4-diaza-6-indancarboxamide (Compound 101)

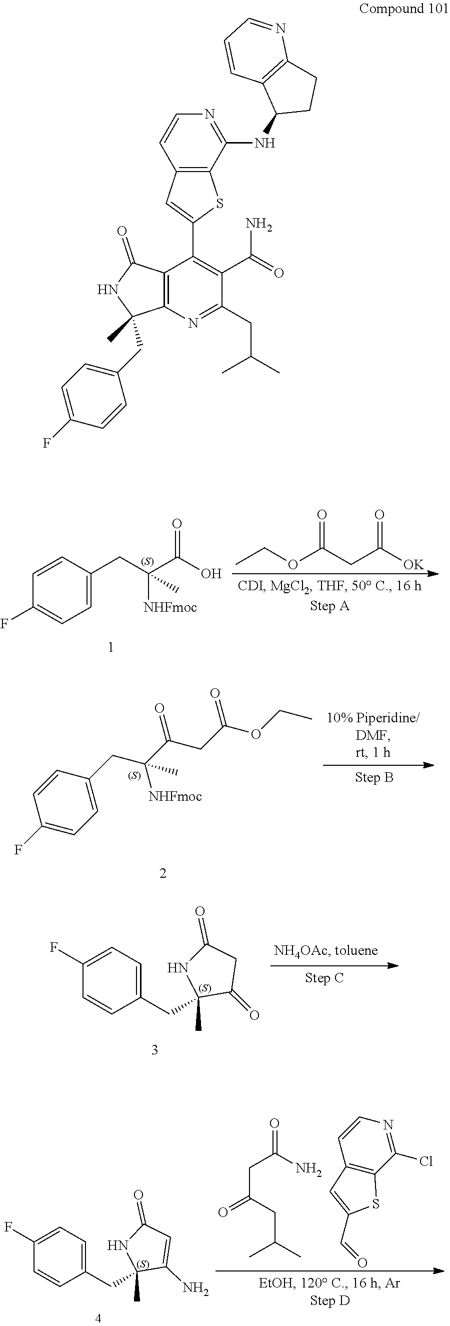

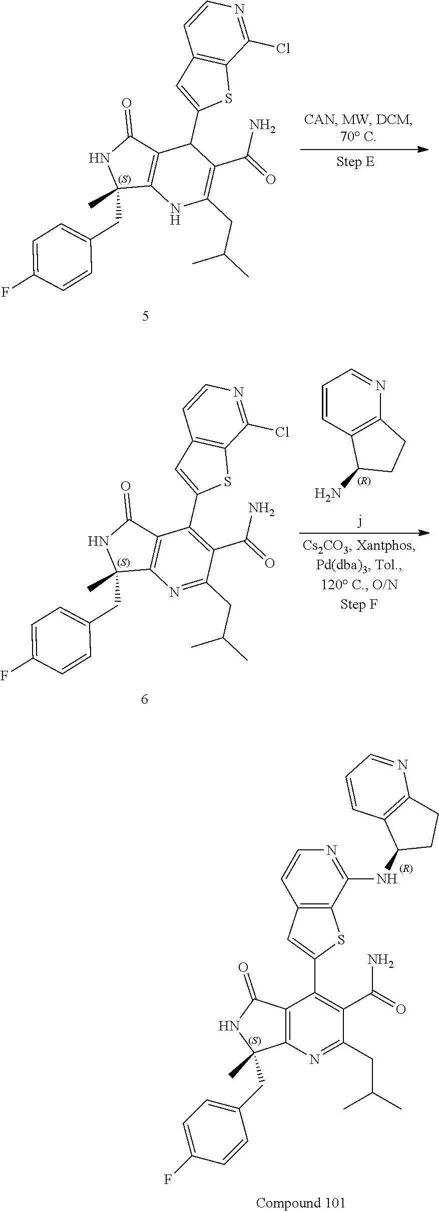

### Step A: ethyl(S)-4-((((9H-fluoren-9-yl) methoxy) carbonyl)amino)-5-(4-fluorophenyl)-4-methyl-3-oxopentanoate

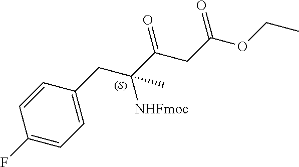

To a solution of (2S)-3-(4-fluorophenyl)-2-({[(9H-fluoren-9-ylmethyl)oxy]carbonyl}amino)-2-methylpropanoic acid (750 mg, 1.788 mmol) in THF (15 mL) was added CDI (434.90 mg, 2.682 mmol), the mixture was stirred at room temperature for 1 hour, [(3-ethoxy-1,3-dioxopropyl)oxy]potassium (608.69 mg, 3.576 mmol) and MgCl₂ (340.48 mg, 3.576 mmol) were added, and the reaction mixture was stirred at 70° C. overnight. The reaction mixture was diluted with EA and brine. The organic layer was separated, washed with brine, and concentrated in vacuum. The residue was purified by silica gel column chromatography eluting with EtOAc:PE=1:5 and concentrated in vacuum to afford ethyl(S)-4-((((9H-fluoren-9-yl) methoxy) carbonyl)amino)-5-(4-fluorophenyl)-4-methyl-3-oxopentanoate (500 mg, 57.12%). LC-MS: m/z 512.1 (M+Na)⁺.

### Step B: (S)-5-(4-fluorobenzyl)-5-methylpyrrolidine-2,4-dione

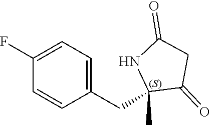

To a solution of ethyl(S)-4-((((9H-fluoren-9-yl) methoxy) carbonyl)amino)-5-(4-fluorophenyl)-4-methyl-3-oxopentanoate (500 mg, 1.021 mmol) in DMF (2 mL) was added piperidine (0.2 mL) and the reaction mixture was stirred at room temperature for 30 min. The residue was purified by reversed phase column with 0.1% FA and concentrated in vacuum to afford (S)-5-(4-fluorobenzyl)-5-methylpyrrolidine-2,4-dione (130 mg, 57.53%). LC-MS: m/z 222.0 (M+H-Boc) 1.

### Step C: (S)-4-amino-5-(4-fluorobenzyl)-5-methyl-1,5-dihydro-2H-pyrrol-2-one

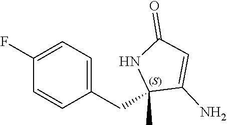

To a solution of ((S)-5-(4-fluorobenzyl)-5-methylpyrrolidine-2,4-dione (130 mg, 0.59 mmol) in toluene (5 mL) was added NH₄OAc (226 mg, 2.94 mmol) and the reaction mixture was stirred at 110° C. overnight with N₂ protection. The reaction mixture was filtered and concentrated in vacuum to afford (S)-4-amino-5-(4-fluorobenzyl)-5-methyl-1,5-dihydro-2H-pyrrol-2-one (160 mg crude) which was used in the next step without further purification. LC-MS: m/z 220.1 (M+H)⁺.

### Step D: (7S)-4-(7-chlorothieno[2,3-c]pyridin-2-yl)-7-[(4-fluorophenyl)methyl]-2-(2-methylpropyl)-5-oxo-1,4,5,6,7,8-hexahydropyrido[4,3-b]pyridine-3-carboxamide

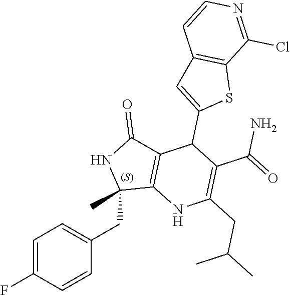

To a solution of(S)-4-amino-5-(4-fluorobenzyl)-5-methyl-1,5-dihydro-2H-pyrrol-2-one (120 mg, 0.54 mmol) in EtOH (5 mL) were added 7-chlorothieno[2,3-c]pyridine-2-carbaldehyde (107 mg, 0.54 mmol), 5-methyl-3-oxohexanamide (77 mg, 0.54 mmol) and the reaction mixture was stirred at 120° C. overnight in a sealed tube with Ar protection. The residue was purified by TLC with DCM:MeOH=20:1 and concentrated in vacuum to afford (7S)-4-(7-chlorothieno[2,3-c]pyridin-2-yl)-7-[(4-fluorophenyl)methyl]-2-(2-methylpropyl)-5-oxo-1,4,5,6,7,8-hexahydropyrido[4,3-b]pyridine-3-carboxamide (63 mg, 22.12%).

### Step E: (S)-4-(7-chlorothieno[2,3-c]pyridin-2-yl)-7-(4-fluorobenzyl)-2-isobutyl-7-methyl-5-oxo-6,7-dihydro-5H-pyrrolo[3,4-b]pyridine-3-carboxamide

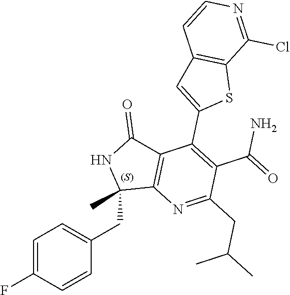

To a solution of (7S)-4-(7-chlorothieno[2,3-c]pyridin-2-yl)-7-[(4-fluorophenyl)methyl]-7-methyl-2-(2-methylpropyl)-5-oxo-4,5,6,7-tetrahydro-1H-pyrrolo[4,3-b]pyridine-3-carboxamide (60 mg, 0.11 mmol) in DCM (4 mL) was added ceric ammonium nitrate (188 mg, 0.34 mmol) and the reaction was stirred at 70° C. for 30 min. The reaction mixture was filtered and concentrated in vacuum to afford (S)-4-(7-chlorothieno[2,3-c]pyridin-2-yl)-7-(4-fluorobenzyl)-2-isobutyl-7-methyl-5-oxo-6,7-dihydro-5H-pyrrolo[3,4-b]pyridine-3-carboxamide (50 mg, 83.66%) which was used in the next step without further purification. LC-MS: m/z 523.3 (M+H)⁺.

### Step F: (S)-7-{7-[(R)-4-aza-1-indanylamino]-1-thia-6-aza-2-indenyl}-3-[(p-fluorophenyl)methyl]-5-isobutyl-3-methyl-1-oxo-2,4-diaza-6-indancarboxamide (Compound 101)

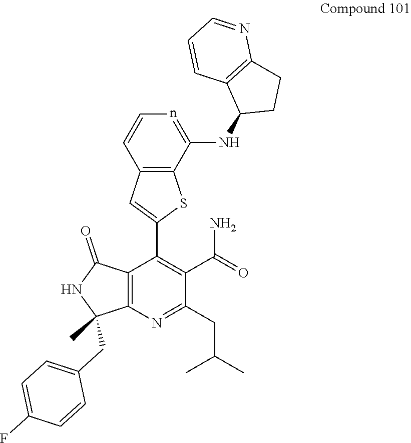

To a mixture of(S)-4-(7-chlorothieno[2,3-c]pyridin-2-yl)-7-(4-fluorobenzyl)-2-isobutyl-7-methyl-5-oxo-6,7-dihydro-5H-pyrrolo[3,4-b]pyridine-3-carboxamide (50 mg, 0.096 mmol) in toluene (5 mL) were added Cs₂CO₃ (62.30 mg, 0.19 mmol), (5R)-6,7-dihydro-5H-cyclopenta[1,2-b]pyridin-5-amine (39 mg, 0.29 mmol), Xantphos (11 mg, 0.019 mmol) and Pd (dba) 3 (9 mg, 0.01 mmol) and the reaction mixture was stirred at 120° C. overnight in a sealed tube with Ar protection. The reaction mixture was filtered and concentrated in vacuum. The residue was purified by TLC with DCM:MeOH=20:1 and Prep-HPLC (0.1% FA) to afford the compound (7S)-7-[(4-fluorophenyl)methyl]-4-(7-{[(5R)-6,7-dihydro-5H-cyclopenta[1,2-b]pyridin-5-yl]amino}thieno[2,3-c]pyridin-2-yl)-7-methyl-2-(2-methylpropyl)-5-oxo-6,7-dihydro-5H-pyrrolo[4,3-b]pyridine-3-carboxamide (1.14 mg, 1.92%).

¹H NMR (400 MHz, CD₃OD) δ 8.33 (d, J=4.4 Hz, 1H), 7.91 (d, J=5.6 Hz, 1H), 7.71 (d, J=7.6 Hz, 1H), 7.22 (s, 1H), 7.18-7.21 (m, 1H), 7.07 (d, J=5.6 Hz, 1H), 6.88-6.92 (m, 2H), 6.81 (t, J=8.8 Hz, 2H), 5.87 (t, J=8.0 Hz, 1H), 3.39-3.42 (m, 1H), 3.12-3.18 (m, 2H), 2.99-3.06 (m, 1H), 2.88-2.97 (m, 3H), 2.68-2.74 (m, 1H), 2.07-2.12 (m, 1H), 1.67 (s, 3H), 1.08 (d, J=6.4 Hz, 3H), 1.22 (d, J=6.8 Hz, 3H). ¹⁹F NMR (376 MHz, CD₃OD) δ −118.24. LC-MS: m/z 621.2 (M+H)⁺.

### Example 2

### (S)-7-{1 [(3,4-difluorophenyl)methyl]-5-indolyl}-3-[(p-fluorophenyl)methyl]-5-isobutyl-3-methyl-1-oxo-2,4-diaza-6-indancarboxamide (Compound 102)

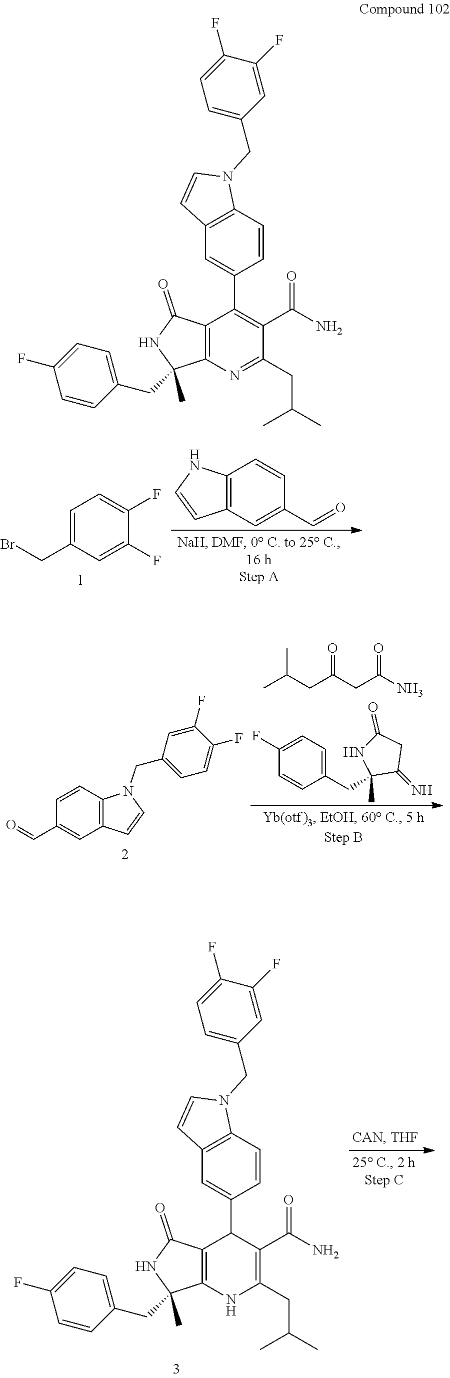

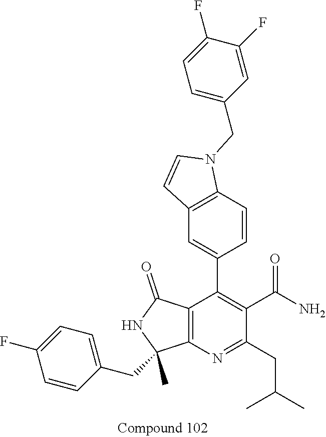 [SMILES: CC(C)Cc1nc2c(c(-c3ccc4c(ccn4Cc4ccc(F)c(F)c4)c3)c1C(N)=O)C(=O)N[C@@]2(C)Cc1ccc(F)cc1]

### Step A: 1-[(3,4-difluorophenyl)methyl]indole-5-carbaldehyde

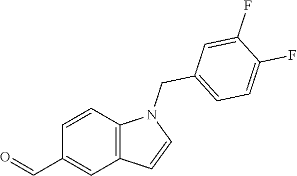 [SMILES: O=Cc1ccc2c(ccn2Cc2ccc(F)c(F)c2)c1]

To a solution of 1H-indole-5-carbaldehyde (500 mg, 3.444 mmol) in DMF (10 mL) was added NaH (206.67 mg, 5.167 mmol) at 0° C. and stirred at 0° C. for 1 hour under N₂. 4-(bromomethyl)-1,2-difluorobenzene (0.529 mL, 4.133 mmol) was then added and the reaction mixture was stirred at 25° C. for another 16 hours under N₂. To the reaction mixture was added EA (50 mL), then the reaction mixture was washed with H₂O (30 mL×3) and brine (20 mL), dried over anhydrous Na₂SO₄, filtered and concentrated under vacuum. The resulting residue was purified by silica gel chromatography (PE/EA=2/1) to afford 1-[(3,4-difluorophenyl)methyl]indole-5-carbaldehyde (912 mg, 97.60%). LC-MS: m/z 272.1 (M+H)⁺.

### Step B: (7S)-4-{1-[(3,4-difluorophenyl)methyl]indol-5-yl}-7-[(4-fluorophenyl)methyl]-7-methyl-2-(2-methylpropyl)-5-oxo-4,5,6,7-tetrahydro-1H-pyrrolo[4,3-b]pyridine-3-carboxamide

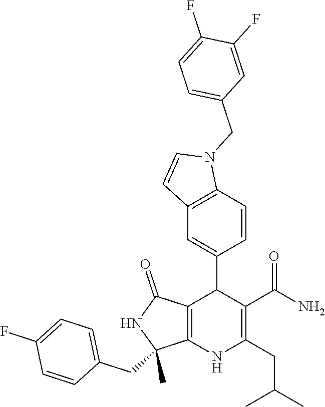 [SMILES: CC(C)CC1=C(C(N)=O)C(c2ccc3c(ccn3Cc3ccc(F)c(F)c3)c2)C2=C(N1)[C@](C)(Cc1ccc(F)cc1)NC2=O]

To a solution of 1-[(3,4-difluorophenyl)methyl]indole-5-carbaldehyde (50 mg, 0.184 mmol), (5S)-4-azanylidene-5-[(4-fluorophenyl)methyl]-5-methyltetrahydropyrrol-2-one (69.01 mg, 0.313 mmol) and 5-methyl-3-oxohexanamide (52.79 mg, 0.369 mmol) in EtOH (5 mL) was added Yb(OTf)₃ (11.41 mg, 0.018 mmol) under N₂. The reaction mixture was stirred at 60° C. for 5 hours in a sealed tube under N₂. The reaction mixture was concentrated under vacuum to afford (7S)-4-{1-[(3,4-difluorophenyl)methyl]indol-5-yl}-7-[(4-fluorophenyl)methyl]-7-methyl-2-(2-methylpropyl)-5-oxo-4,5,6,7-tetrahydro-1H-pyrrolo[4,3-b]pyridine-3-carboxamide (60 mg, 54.37%) which was used directly in the next step without further purification. LC-MS: m/z 599.3 (M+H)⁺.

### Step C: (S)-7-{1-[(3,4-difluorophenyl)methyl]-5-indolyl}-3-[(p-fluorophenyl)methyl]-5-isobutyl-3-methyl-1-oxo-2,4-diaza-6-indancarboxamide (Compound 102)

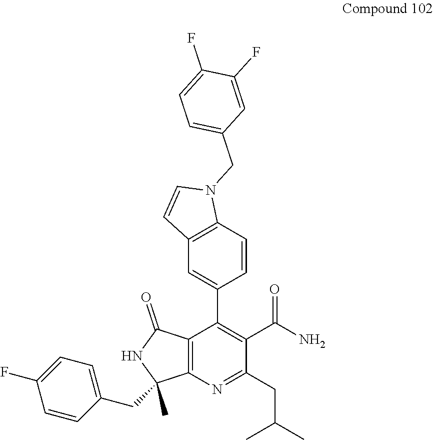 [SMILES: CC(C)Cc1nc2c(c(-c3ccc4c(ccn4Cc4ccc(F)c(F)c4)c3)c1C(N)=O)C(=O)N[C@@]2(C)Cc1ccc(F)cc1]

To a solution of (7S)-4-{1-[(3,4-difluorophenyl)methyl]indol-5-yl}-7-[(4-fluorophenyl)methyl]-7-methyl-2-(2-methylpropyl)-5-oxo-4,5,6,7-tetrahydro-1H-pyrrolo[4,3-b]pyridine-3-carboxamide (60 mg, 0.100 mmol) in THF (5 mL) was added ceric ammonium nitrate (183.15 mg, 0.334 mmol), and the reaction mixture was stirred at 25° C. for 2 hours under N₂. To the reaction mixture was added EA (20 mL), then the reaction mixture was washed with aq. NaHCO₃ (10 mL), the organic layer was separated and dried over Na₂SO₄, filtered, and concentrated under vacuum. The resulting residue was purified by prep-HPLC (Waters 3767/Qda Column: SunFire Sunfire C18, 19*250 mm*10 μm; Mobile Phase A: 0.1% FA/H₂O, B: ACN; Flow rate: 20 ml/min; Gradient: 58%-63%; Retention Time: 9.2-10.1 min of 17 min) to afford (S)-7-{1-[(3,4-difluorophenyl)methyl]-5-indolyl}-3-[(p-fluorophenyl)methyl]-5-isobutyl-3-methyl-1-oxo-2,4-diaza-6-indancarboxamide (15.15 mg, 14.66%).

¹H NMR (400 MHz, DMSO-d₆) δ 8.57 (s, 1H), 7.53-7.56 (m, 2H), 7.29-7.42 (m, 5H), 7.11-7.14 (m, 1H), 6.82-6.93 (m, 5H), 6.44 (d, J=2.8 Hz, 1H), 5.39 (s, 2H), 3.26 (d, J=13.6 Hz, 1H), 3.05 (d, J=13.2 Hz, 1H), 2.74-2.78 (m, 2H), 2.37-2.40 (m, 1H), 1.55 (s, 3H), 1.03 (d, J=6.8 Hz, 3H), 0.92 (d, J=6.8 Hz, 3H). ¹⁹F NMR (376 MHz, DMSO-d₆) δ −116.78, −138.37, −140.42. LC-MS: m/z 597.2 (M+H)⁺.

### (S)-7-{1-[(4-cyano-3-fluorophenyl)methyl]-5-indolyl}-3-[(p-fluorophenyl)methyl]-5-isobutyl-3-methyl-1-oxo-2,4-diaza-6-indancarboxamide (Compound 103)

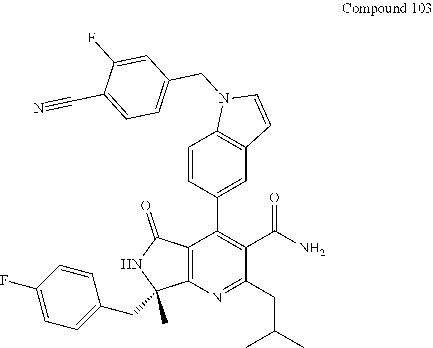 [SMILES: CC(C)Cc1nc2c(c(-c3ccc4c(ccn4Cc4ccc(C#N)c(F)c4)c3)c1C(N)=O)C(=O)N[C@@]2(C)Cc1ccc(F)cc1]

Compound 103 was then synthesized following the similar route of Example 2, using 4-(Bromomethyl)-2-fluorobenzonitrile in step A.

¹H NMR (400 MHz, DMSO-d₆) δ 8.57 (s, 1H), 7.88 (t, J=7.2 Hz, 1H), 7.55-7.57 (m, 2H), 7.41 (d, J=10.0 Hz, 1H), 7.30-7.36 (m, 3H), 7.21 (d, J=8.0 Hz, 1H), 6.91 (t, J=8.8 Hz, 2H), 6.82-6.86 (m, 3H), 6.47 (d, J=2.8 Hz, 1H), 5.53 (s, 2H), 3.25 (d, J=13.2 Hz, 1H), 3.04 (d, J=13.2 Hz, 1H), 2.72-2.81 (m, 2H), 2.35-2.42 (m, 1H), 1.55 (s, 3H), 1.02 (d, J=6.4 Hz, 3H), 0.92 (d, J=6.4 Hz, 3H). ¹⁹F NMR (376 MHz, DMSO-d₆) δ −108.22, −116.78. LC-MS: m/z 604.4 (M+H)⁺.

### (S)-3-[(p-fluorophenyl)methyl]-5-isobutyl-3-methyl-1-oxo-7-{1-[(2-pyridyl)methyl]-5-indolyl}-2,4-diaza-6-indancarboxamide (Compound 109)

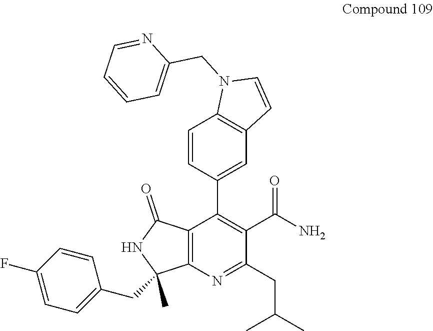 [SMILES: CC(C)Cc1nc2c(c(-c3ccc4c(ccn4Cc4ccccn4)c3)c1C(N)=O)C(=O)N[C@@]2(C)Cc1ccc(F)cc1]

Compound 109 was then synthesized following the similar route of Example 2, using picolyl chloride hydrochloride in step A.

¹H NMR (400 MHz, DMSO-d₆) δ 8.55-8.57 (m, 2H), 7.76-7.80 (m, 1H), 7.56 (s, 1H), 7.50 (d, J=3.2 Hz, 1H), 7.29-7.34 (m, 4H), 7.16 (d, J=7.6 Hz, 1H), 6.82-6.93 (m, 5H), 6.45 (d, J=2.8 Hz, 1H), 5.50 (s, 2H), 3.26 (d, J=13.2 Hz, 1H), 3.05 (d, J=13.2 Hz, 1H), 2.70-2.81 (m, 2H), 2.37-2.42 (m, 1H), 1.55 (s, 3H), 1.03 (d, J=6.8 Hz, 3H), 0.93 (d, J=6.8 Hz, 3H). ¹⁹F NMR (376 MHz, DMSO-d₆) δ −116.79. LC-MS: m/z 562.4 (M+H)⁺.

### (S)-7-{1-[(3-cyano-4-fluorophenyl)methyl]-5-indolyl}-3-[(p-fluorophenyl)methyl]-5-isobutyl-3-methyl-1-oxo-2,4-diaza-6-indancarboxamide (Compound 156)

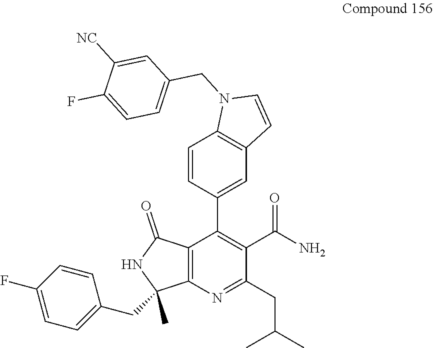 [SMILES: CC(C)Cc1nc2c(c(-c3ccc4c(ccn4Cc4ccc(F)c(C#N)c4)c3)c1C(N)=O)C(=O)N[C@@]2(C)Cc1ccc(F)cc1]

Compound 156 was synthesized following the similar route of Example 2, using 5-(bromomethyl)-2-fluorobenzonitrile in step A.

¹H NMR (400 MHz, DMSO-d₆) δ 8.57 (s, 1H), 7.95 (d, J=4.4 Hz, 1H), 7.70-7.66 (m, 1H), 7.57 (d, J=3.2 Hz, 2H), 7.48 (t, J=8.8 Hz, 1H), 7.42 (d, J=8.4 Hz, 1H), 7.33-7.29 (m, 2H), 6.91 (t, J=8.8 Hz, 2H), 6.86-6.82 (m, 3H), 6.45 (d, J=2.8 Hz, 1H), 5.43 (s, 2H), 3.24 (d, J=13.2 Hz, 1H), 3.04 (d, J=13.2 Hz, 1H), 2.81-2.70 (m, 2H), 2.02-1.98 (m, 1H), 1.55 (s, 3H), 1.02 (d, J=6.4 Hz, 3H), 0.92 (d, J=6.4 Hz, 3H). ¹⁹F NMR (376 MHz, DMSO-d₆) δ −110.19, −116.78. LC-MS: m/z 604.3 (M+H)⁺.

### (S)-7-{1-[(3,4-difluorophenyl)methyl]-1H-1,7-diazainden-5-yl}-3-[(p-fluorophenyl)methyl]-5-isobutyl-3-methyl-1-oxo-2,4-diaza-6-indancarboxamide (Compound 157)

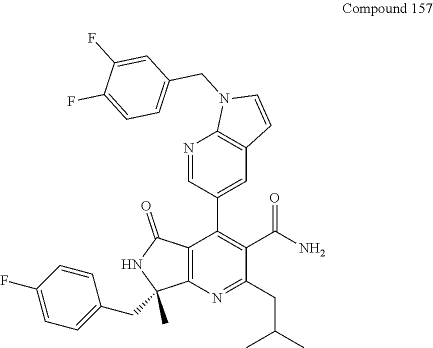 [SMILES: CC(C)Cc1nc2c(c(-c3cnc4c(ccn4Cc4ccc(F)c(F)c4)c3)c1C(N)=O)C(=O)N[C@@]2(C)Cc1ccc(F)cc1]

Compound 157 was synthesized following the similar route of Example 2, using 1H-pyrrolo[2,3-b]pyridine-5-carbaldehyde in step A.

¹H NMR (400 MHz, DMSO-d₆) δ 70 (s, 1H), 7.93 (s, 1H), 7.74-7.67 (m, 3H), 7.42-7.34 (m, 3H), 7.13-7.11 (m, 1H), 6.92 (t, J=8.8 Hz, 2H), 6.88-6.83 (m, 2H), 6.52 (d, J=3.2 Hz, 1H), 5.45 (s, 2H), 3.26 (d, J=13.2 Hz, 1H), 3.06 (d, J=13.2 Hz, 1H), 2.82-2.73 (m, 2H), 2.42-2.37 (m, 1H), 1.57 (s, 3H), 1.04 (d, J=6.8 Hz, 3H), 0.94 (d, J=6.8 Hz, 3H). ¹⁹F NMR (376 MHz, DMSO-d₆) δ −116.75, −138.36, −140.42. LC-MS: m/z 598.1 (M+H)⁺.

### (S)-7-{1-[2-(3,4-difluorophenyl)ethyl]-5-indolyl}-3-[(p-fluorophenyl)methyl]-5-isobutyl-3-methyl-1-oxo-2,4-diaza-6-indancarboxamide (Compound 104)

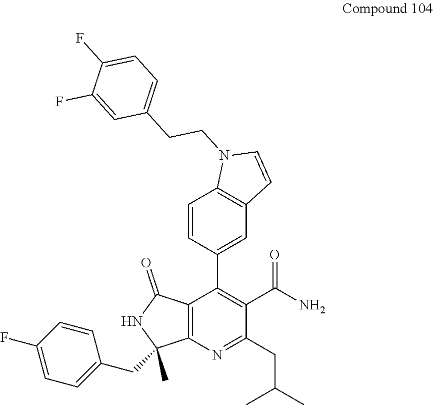 [SMILES: CC(C)Cc1nc2c(c(-c3ccc4c(ccn4CCc4ccc(F)c(F)c4)c3)c1C(N)=O)C(=O)N[C@@]2(C)Cc1ccc(F)cc1]

Compound 104 was then synthesized following the similar route of Example 2, using 4-(2-Bromoethyl)-1,2-difluorobenzene in stop A.

¹H NMR (400 MHz, DMSO-d₆) δ 8.57 (s, 1H), 7.57 (s, 1H), 7.47-7.40 (m, 2H), 7.36-7.29 (m, 4H), 7.11-7.08 (m, 1H), 6.94-6.84 (m, 5H), 6.35 (d, J=3.2 Hz, 1H), 4.38 (t, J=7.6 Hz, 2H), 3.26 (d, J=13.2 Hz, 1H), 3.08-3.03 (m, 3H), 2.82-2.72 (m, 2H), 2.43-2.36 (m, 1H), 1.56 (s, 3H), 1.03 (d, J=6.8 Hz, 3H), 0.94 (d, J=6.8 Hz, 3H). ¹⁹F NMR (376 MHz, DMSO-d₆) δ −118.01. LC-MS: m/z 611.0 (M+H)⁺.

### Example 3

### (S)-7-{1-[1-(3,4-difluorophenyl)ethyl]-5-indolyl}-3-[(p-fluorophenyl)methyl]-5-isobutyl-3-methyl-1-oxo-2,4-diaza-6-indancarboxamide (Compound 105)

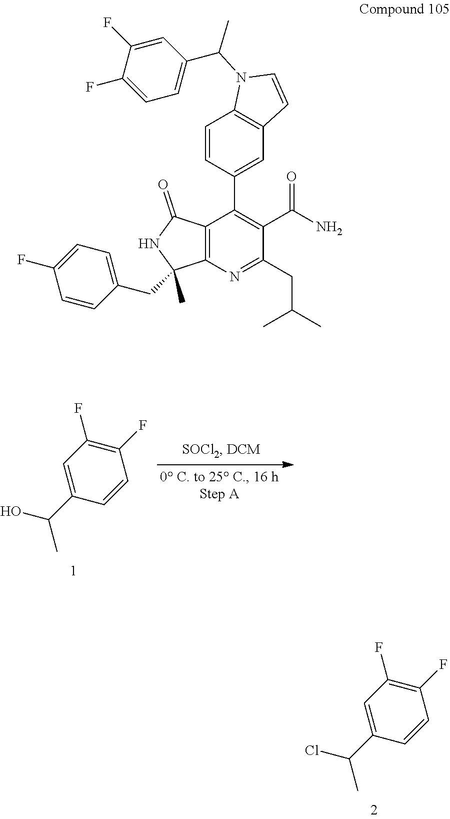 [SMILES: CC(C)Cc1nc2c(c(-c3ccc4c(ccn4C(C)c4ccc(F)c(F)c4)c3)c1C(N)=O)C(=O)N[C@@]2(C)Cc1ccc(F)cc1 ; CC(O)c1ccc(F)c(F)c1 ; CC(Cl)c1ccc(F)c(F)c1]

### Step A: 4-(1-chloroethyl)-1,2-difluorobenzene

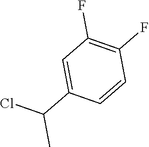

To a solution of 1-(3,4-difluorophenyl) ethan-1-ol (500 mg, 3.162 mmol) in DCM (10 mL) was added chloranesulfinyl chloride (0.459 mL, 6.324 mmol) at 0° C., and the reaction mixture was stirred at 25° C. for 2 hours. The reaction mixture was concentrated in vacuo. The resulting residue was purified by TLC (PE/EA=2/1) to afford 4-(1-chloroethyl)-1,2-difluorobenzene (256 mg, 45.85%). LC-MS: m/z 200.2 (M+Na)⁺.

### (S)-7-{1-[1-(3,4-difluorophenyl)ethyl]-5-indolyl}-3-[(p-fluorophenyl)methyl]-5-isobutyl-3-methyl-1-oxo-2,4-diaza-6-indancarboxamide (Compound 105)

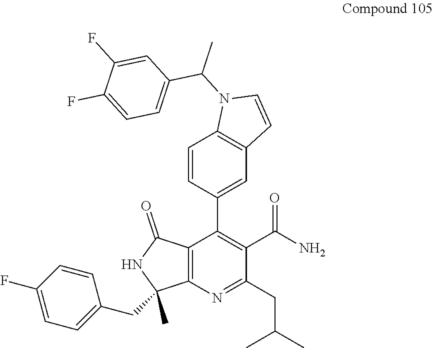 [SMILES: CC(C)Cc1nc2c(c(-c3ccc4c(ccn4C(C)c4ccc(F)c(F)c4)c3)c1C(N)=O)C(=O)N[C@@]2(C)Cc1ccc(F)cc1]

Compound 105 was then synthesized following the similar route of Example 2, using 4-(1-chloroethyl)-1,2-difluorobenzene in step A.

¹H NMR (400 MHz, DMSO-d₆) δ 8.57 (s, 1H), 7.71 (d, J=3.2 Hz, 1H), 7.55 (s, 1H), 7.43-7.50 (m, 1H), 7.34-7.39 (m, 2H), 7.28-7.34 (m, 2H), 7.15-7.21 (m, 1H), 6.88-6.92 (m, 2H), 6.83-6.85 (m, 3H), 6.47 (d, J=3.2 Hz, 1H), 5.81-5.86 (m, 1H), 3.24 (d, J=13.6 Hz, 1H), 3.03 (d, J=13.6 Hz, 1H), 2.73-2.77 (m, 2H), 2.35-2.41 (m, 1H), 1.86 (d, J=7.2 Hz, 3H), 1.54 (s, 3H), 1.02 (d, J=6.8 Hz, 3H), 0.91 (d, J=6.8 Hz, 3H). ¹⁹F NMR (376 MHz, DMSO-d₆) δ −116.79, −138.19, −140.49. LC-MS: m/z 611.3 (M+H)⁺.

### (S)-3-[(p-fluorophenyl)methyl]-5-isobutyl-3-methyl-7-{1-[(6-methyl-3-pyridyl)methyl]-5-indolyl}-1-oxo-2,4-diaza-6-indancarboxamide (Compound 154)

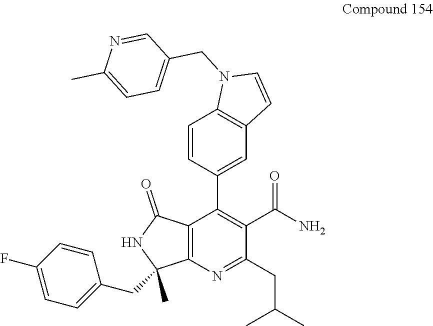 [SMILES: Cc1ccc(Cn2ccc3cc(-c4c(C(N)=O)c(CC(C)C)nc5c4C(=O)N[C@@]5(C)Cc4ccc(F)cc4)ccc32)cn1]

Compound 154 was synthesized following the similar route of Example 3, using (6-methylpyridin-3-yl) methanol in step A.

¹H NMR (400 MHz, DMSO-d₆) δ 8.56 (s, 1H), 8.52-8.51 (m, 1H), 7.67 (d, J=7.6 Hz, 1H), 7.55-7.54 (m, 2H), 7.40 (d, J=8.8 Hz, 1H), 7.32-7.28 (m, 2H), 7.21-7.19 (m, 1H), 6.90 (t, J=8.8 Hz, 2H), 6.85-6.82 (m, 3H), 6.43 (d, J=2.8 Hz, 1H), 5.41 (s, 2H), 3.24 (d, J=13.6 Hz, 1H), 3.03 (d, J=13.6 Hz, 1H), 2.79-2.73 (m, 2H), 2.44 (s, 3H), 2.40-2.36 (m, 1H), 1.55 (s, 3H), 1.02 (d, J=6.4 Hz, 3H), 0.92 (d, J=6.4 Hz, 3H). ¹⁹F NMR (376 MHz, DMSO-d₆) δ −116.79. LC-MS: m/z 576.4 (M+H)⁺.

### (S)-3-[(p-fluorophenyl)methyl]-5-isobutyl-3-methyl-7-{1-[(2-methyl-4-pyridyl)methyl]-5-indolyl}-1-oxo-2,4-diaza-6-indancarboxamide (Compound 155)

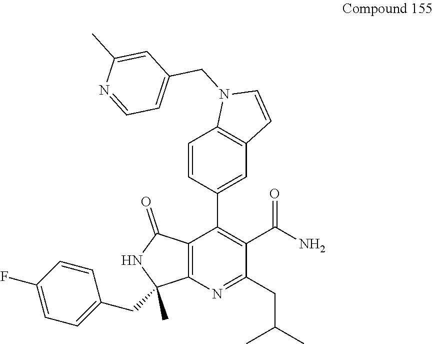 [SMILES: Cc1cc(Cn2ccc3cc(-c4c(C(N)=O)c(CC(C)C)nc5c4C(=O)N[C@@]5(C)Cc4ccc(F)cc4)ccc32)ccn1]

Compound 155 was synthesized following the similar route of Example 3, using (2-methylpyridin-4-yl) methanol in step A.

¹H NMR (400 MHz, DMSO-d₆) δ 8.57 (s, 1H), 8.54-8.53 (m, 1H), 7.58 (s, 1H), 7.54 (d, J=3.2 Hz, 1H), 7.40-7.37 (m, 2H), 7.31-7.29 (m, 2H), 7.26-7.24 (m, 1H), 6.90 (t, J=9 Hz, 2H), 6.85-6.82 (m, 3H), 6.50 (d, J=2.8 Hz, 1H), 5.56 (s, 2H), 3.25 (d, J=13.6 Hz, 1H), 3.02 (d, J=13.6 Hz, 1H), 2.78-2.74 (m, 2H), 2.53 (s, 3H), 2.40-2.37 (m, 1H), 1.55 (s, 3H), 1.02 (d, J=6.8 Hz, 3H), 0.92 (d, J=6.8 Hz, 3H). ¹⁹F NMR (376 MHz, DMSO-d₆) δ −116.79. LC-MS: m/z 576.4 (M+H)⁺.

### Example 4

### (S)-7-{1-[1-(3,4-difluorophenyl)cyclopropyl]-5-indolyl}-3-[(p-fluorophenyl)methyl]-5-isobutyl-3-methyl-1-oxo-2,4-diaza-6-indancarboxamide (Compound 106)

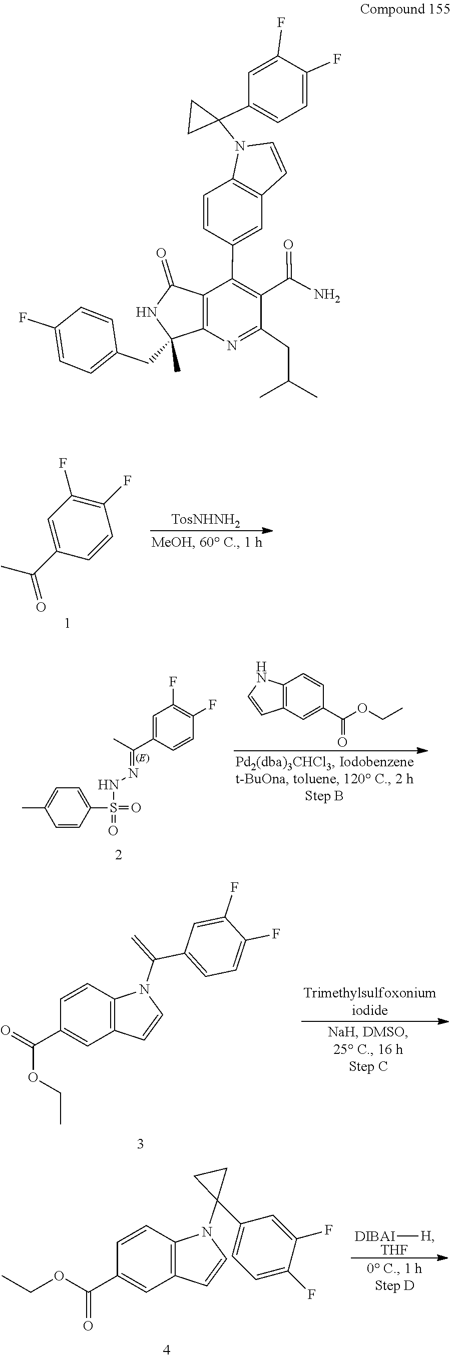

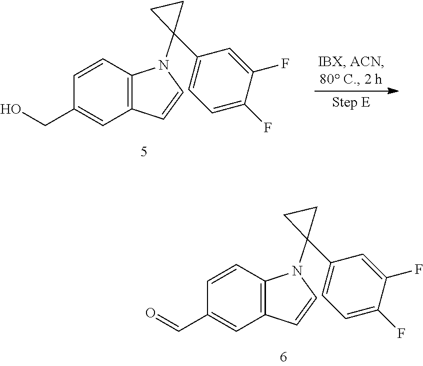 [SMILES: OCc1ccc2c(ccn2C2(c3ccc(F)c(F)c3)CC2)c1 ; O=Cc1ccc2c(ccn2C2(c3ccc(F)c(F)c3)CC2)c1]

### Step A: (E)-N′-(1-(3,4-difluorophenyl)ethylidene)-4-methylbenzenesulfonohydrazide

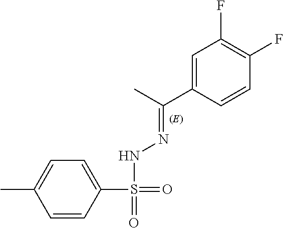 [SMILES: C/C(=N\NS(=O)(=O)c1ccc(C)cc1)c1ccc(F)c(F)c1]

To a solution of 1-(3,4-difluorophenyl) ethan-1-one (5.0 g, 32.025 mmol) in MeOH (60 mL) was added N-amino-4-methylbenzenesulfonamide (5.96 g, 32.025 mmol), the mixture was stirred at 60° C. for 1 hour under N₂. The mixture was concentrated under vacuum to afford (E)-N′-(1-(3,4-difluorophenyl)ethylidene)-4-methylbenzenesulfonohydrazide (10.02 g, 96.47%) which was used in the next step without further purification. LC-MS: m/z 325.2 (M+H)⁺.

### Step B: ethyl 1-(1-(3,4-difluorophenyl) vinyl)-1H-indole-5-carboxylate

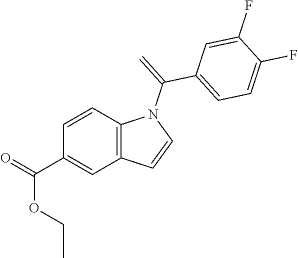 [SMILES: C=C(c1ccc(F)c(F)c1)n1ccc2cc(C(=O)OCC)ccc21]

To a solution of ethyl 1H-indole-5-carboxylate (500 mg, 2.643 mmol) in DMF (12 mL) were added (E)-N′-(1-(3,4-difluorophenyl)ethylidene)-4-methylbenzenesulfonohydrazide (1285.67 mg, 3.964 mmol), palladium chloride bis(triphenylphosphane) (185.51 mg, 0.264 mmol) and lithium 2-methylpropan-2-olate (423.20 mg, 5.286 mmol), the reaction mixture was stirred at 80° C. for 16 hours under air. To the mixture was added water (100 mL), the resulting mixture was extracted with EA (50 mL×2), the combined organic layers were washed with brine (50 mL×2), dried over anhydrous Na₂SO₄, filtered and concentrated under vacuum. The resulting residue was purified by flash column chromatography (eluting PE/EA=12/1) to afford ethyl 1-(1-(3,4-difluorophenyl) vinyl)-1H-indole-5-carboxylate (420 mg, 48.56%).

LC-MS: m/z 328.1 (M+H)⁺. ¹H NMR (400 MHz, CDCl₃) δ 8.41 (d, J=1.6 Hz, 1H), 7.82-7.84 (dd, J=8.8, 1.6 Hz, 1H), 7.19-7.23 (m, 2H), 7.07-7.16 (m, 2H), 6.98-7.01 (m, 1H), 6.72 (d, J=3.2 Hz, 1H), 5.63 (s, 1H), 5.43 (s, 1H), 4.36-4.41 (m, 2H), 1.40 (t, J=7 Hz, 3H).

### Step C: ethyl 1-(1-(3,4-difluorophenyl)cyclopropyl)-1H-indole-5-carboxylate

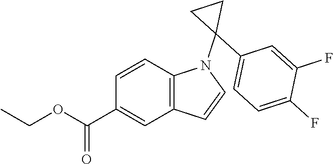 [SMILES: CCOC(=O)c1ccc2c(ccn2C2(c3ccc(F)c(F)c3)CC2)c1]

To a solution of trimethylsulfoxonium iodide (567.34 mg, 2.566 mmol) in DMSO (20 mL) was added NaH (102.65 mg, 2.566 mmol), the mixture was stirred at 25° C. for 30 min under Ar, then ethyl 1-(1-(3,4-difluorophenyl) vinyl)-1H-indole-5-carboxylate (420 mg, 1.283 mmol) was added. The reaction mixture was stirred at 25° C. for 16 hours under Ar. To the mixture was added water (50 mL), the resulting mixture was extracted with EA (50 mL×2), the combined organic layers were washed with brine (100 mL×3), dried over anhydrous Na₂SO₄, filtered and concentrated under vacuum. The resulting residue was purified by flash column chromatography (eluting PE/EA=10/1) to afford ethyl 1-(1-(3,4-difluorophenyl)cyclopropyl)-1H-indole-5-carboxylate (150 mg, 34.25%). LC-MS: m/z 342.1 (M+H)⁺.

### Step D: (1-(1-(3,4-difluorophenyl)cyclopropyl)-1H-indol-5-yl) methanol

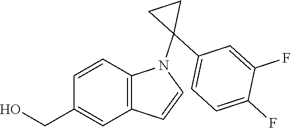 [SMILES: OCc1ccc2c(ccn2C2(c3ccc(F)c(F)c3)CC2)c1]

To a solution of ethyl 1-(1-(3,4-difluorophenyl)cyclopropyl)-1H-indole-5-carboxylate (150 mg, 0.439 mmol) in DCM (10 mL) was added DIBAL-H (1.5 M in toluene, 0.293 mL, 0.439 mmol) at 0° C. under N₂, the reaction mixture was stirred at 0° C. for 1 hour under N₂. The reaction mixture was quenched by 15% citric acid (0.6 mL), the mixture was stirred at room temperature for 30 min, filtered by Celite, the filtrate was extracted with DCM (10 mL×2), the combined organic layers were concentrated under vacuum to afford (1-(1-(3,4-difluorophenyl)cyclopropyl)-1H-indol-5-yl) methanol (132 mg, 100.36%) which was used in the next step without further purification.

### Step E: 1-(1-(3,4-difluorophenyl)cyclopropyl)-1H-indole-5-carbaldehyde

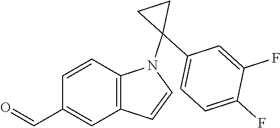 [SMILES: O=Cc1ccc2c(ccn2C2(c3ccc(F)c(F)c3)CC2)c1]

To a solution of (1-(1-(3,4-difluorophenyl)cyclopropyl)-1H-indol-5-yl) methanol (132 mg, 0.441 mmol) in ACN (10 mL) were added IBX (246.98 mg, 0.882 mmol), the reaction mixture was stirred at 80° C. for 2 hours under N₂. The reaction mixture was filtered after cooled to 25° C., and the filtrate was concentrated under vacuum. The resulting residue was purified by prep-TLC (eluting PE/EA=4/1) to afford 1-(1-(3,4-difluorophenyl)cyclopropyl)-1H-indole-5-carbaldehyde (90 mg, 68.64%). LC-MS: m/z 298.1 (M+H)⁺.

### (S)-7-{1-[1-(3,4-difluorophenyl)cyclopropyl]-5-indolyl}-3-[(p-fluorophenyl)methyl]-5-isobutyl-3-methyl-1-oxo-2,4-diaza-6-indancarboxamide (Compound 106)

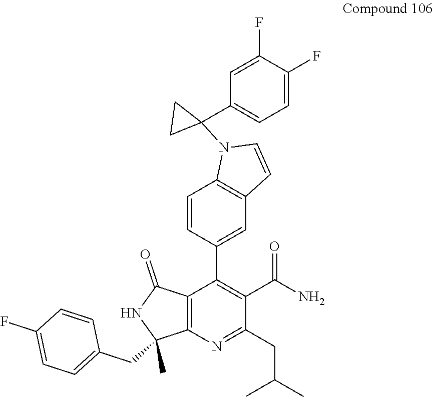 [SMILES: CC(C)Cc1nc2c(c(-c3ccc4c(ccn4C4(c5ccc(F)c(F)c5)CC4)c3)c1C(N)=O)C(=O)N[C@@]2(C)Cc1ccc(F)cc1]

Compound 106 was then synthesized following the similar route of Example 2, using 1-(1-(3,4-difluorophenyl)cyclopropyl)-1H-indole-5-carbaldehyde in step B.

¹H NMR (400 MHz, DMSO-d₆) δ 8.58 (s, 1H), 7.58 (d, J=3.2 Hz, 2H), 7.26-7.36 (m, 4H), 7.04-7.09 (m, 1H), 6.83-6.93 (m, 5H), 6.71-6.74 (m, 1H), 6.47 (d, J=2.8 Hz, 1H), 3.24 (d, J=13.2 Hz, 1H), 3.04 (d, J=13.2 Hz, 1H), 2.74-2.80 (m, 2H), 2.37-2.42 (m, 1H), 1.59-1.67 (m, 4H), 1.55 (s, 3H), 1.03 (d, J=6.8 Hz, 3H), 0.93 (d, J=6.8 Hz, 3H). ¹⁹F NMR (376 MHz, DMSO-d₆) δ −116.72, −138.37, −141.46. LC-MS: m/z 623.8 (M+H)⁺.

### Example 5

### (S)-7-{1-[(3,4-difluorophenyl)methyl]-1H-indazol-5-yl}-3-[(p-fluorophenyl)methyl]-5-isobutyl-3-methyl-1-oxo-2,4-diaza-6-indancarboxamide (Compound 107) and (S)-7-{2-[(3,4-difluorophenyl)methyl]-2H-indazol-5-yl}-3-[(p-fluorophenyl)methyl]-5-isobutyl-3-methyl-1-oxo-2,4-diaza-6-indancarboxamide (Compound 108)

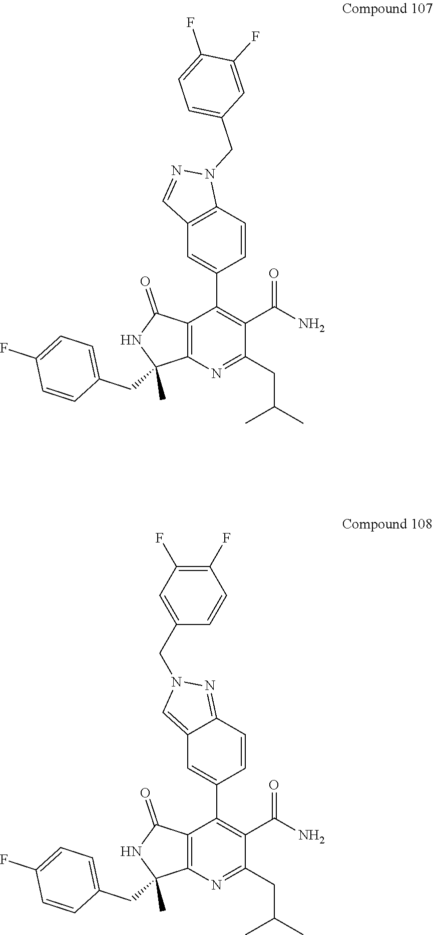 [SMILES: CC(C)Cc1nc2c(c(-c3ccc4c(cnn4Cc4ccc(F)c(F)c4)c3)c1C(N)=O)C(=O)N[C@@]2(C)Cc1ccc(F)cc1 ; CC(C)Cc1nc2c(c(-c3ccc4nn(Cc5ccc(F)c(F)c5)cc4c3)c1C(N)=O)C(=O)N[C@@]2(C)Cc1ccc(F)cc1]

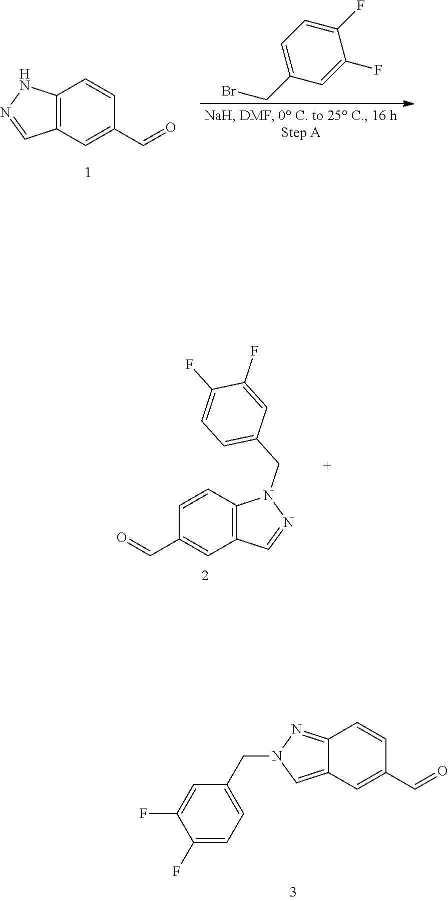 [SMILES: O=Cc1ccc2[nH]ncc2c1 ; Fc1ccc(CBr)cc1F ; O=Cc1ccc2c(cnn2Cc2ccc(F)c(F)c2)c1 ; O=Cc1ccc2nn(Cc3ccc(F)c(F)c3)cc2c1]

### Step A: 1-[(3,4-difluorophenyl)methyl]indazole-5-carbaldehyde and 2-(3,4-difluorobenzyl)-2H-indazole-5-carbaldehyde

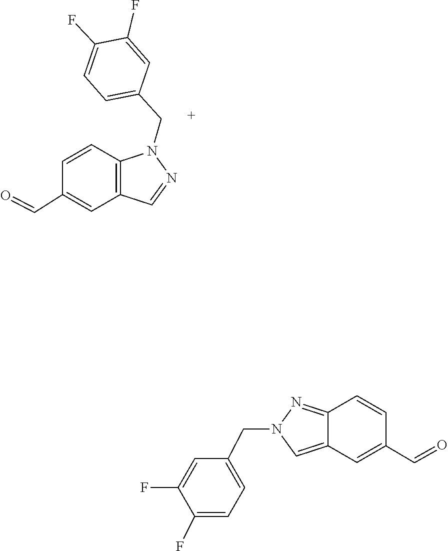 [SMILES: O=Cc1ccc2c(cnn2Cc2ccc(F)c(F)c2)c1 ; O=Cc1ccc2nn(Cc3ccc(F)c(F)c3)cc2c1]

To a solution of 1H-indazole-5-carbaldehyde (470.65 mg, 3.220 mmol) in DMF (10 mL) was added NaH (154.57 mg, 6.441 mmol) at 0° C. and the reaction mixture was stirred at 0° C. for 1 hour under N₂. 4-(bromomethyl)-1,2-difluorobenzene (1 g, 4.830 mmol) was added. The reaction mixture was stirred at 25° C. for another 16 hours under N₂. To the reaction mixture was added EA (50 mL), then the reaction mixture was washed with H₂O (30 mL×3) and brine (20 mL), dried over anhydrous Na₂SO₄, filtered and concentrated under vacuum. The resulting residue was purified by silica gel chromatography (PE/EA=2/1) to afford 1-[(3,4-difluorophenyl)methyl]indazole-5-carbaldehyde (546 mg, 62.28%) and 2-(3,4-difluorobenzyl)-2H-indazole-5-carbaldehyde (53 mg, 6.05%). LC-MS: m/z 273.0 (M+H)⁺.

1-[(3,4-difluorophenyl)methyl]indazole-5-carbaldehyde: ¹H NMR (400 MHz, DMSO-d₆) δ 10.04 (s, 1H), 8.46 (s, 1H), 8.41 (s, 1H), 7.88-7.95 (m, 2H), 7.33-7.42 (m, 2H), 7.07-7.10 (m, 1H), 5.74 (s, 2H).

2-(3,4-difluorobenzyl)-2H-indazole-5-carbaldehyde: ¹H NMR (400 MHz, DMSO-d₆) δ 9.97 (s, 1H), 8.89 (s, 1H), 8.48 (s, 1H), 7.65-7.72 (dd, J=17.6, 9.2 Hz, 2H), 7.40-7.53 (m, 2H), 7.22-7.25 (m, 1H), 5.71 (s, 2H).

### (S)-7-{1-[(3,4-difluorophenyl)methyl]-1H-indazol-5-yl}-3-[(p-fluorophenyl)methyl]-5-isobutyl-3-methyl-1-oxo-2,4-diaza-6-indancarboxamide (Compound 107)

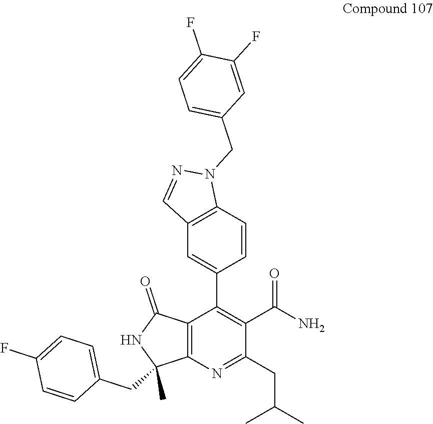 [SMILES: CC(C)Cc1nc2c(c(-c3ccc4c(cnn4Cc4ccc(F)c(F)c4)c3)c1C(N)=O)C(=O)N[C@@]2(C)Cc1ccc(F)cc1]

Compound 107 was then synthesized following the similar route of Example 2, using 1-[(3,4-difluorophenyl)methyl]indazole-5-carbaldehyde in step B.

¹H NMR (400 MHz, DMSO-d₆) δ 8.65 (s, 1H), 8.12 (s, 1H), 7.66 (d. J=8.8 Hz, 2H), 7.46-7.60 (m, 1H), 7.35-7.41 (m, 3H), 7.03-7.22 (m, 2H), 6.91 (t, J=8.8 Hz, 2H), 6.82-6.88 (m, 2H), 5.64 (s, 2H), 3.23 (m, 1H), 3.05 (d, J=13.2 Hz, 1H), 2.73-2.83 (m, 2H), 1.97-2.02 (m, 1H), 1.56 (s, 3H), 1.03 (d, J=6.8 Hz, 3H), 0.93 (d, J=6.8 Hz, 3H). ¹⁹F NMR (376 MHz, DMSO-d₆) δ −116.69, −138.26, −140.07. LC-MS: m/z 598.3 (M+H)⁺.

### (S)-7-{2-[(3,4-difluorophenyl)methyl]-2H-indazol-5-yl}-3-[(p-fluorophenyl)methyl]-5-isobutyl-3-methyl-1-oxo-2,4-diaza-6-indancarboxamide (Compound 108)

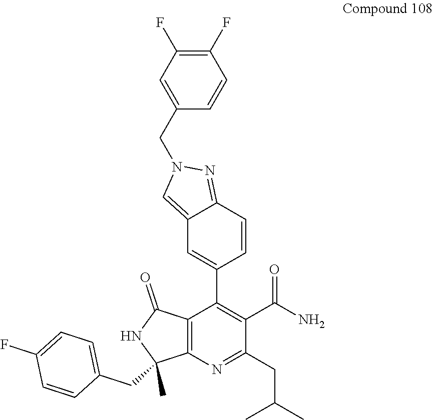 [SMILES: CC(C)Cc1nc2c(c(-c3ccc4nn(Cc5ccc(F)c(F)c5)cc4c3)c1C(N)=O)C(=O)N[C@@]2(C)Cc1ccc(F)cc1]

Compound 108 was then synthesized following the similar route of Example 2, using 2-(3,4-difluorobenzyl)-2H-indazole-5-carbaldehyde in step B.

¹H NMR (400 MHz, DMSO-d₆) δ 8.63 (s, 1H), 8.51 (s, 1H), 7.63 (s, 1H), 7.37-7.50 (m, 4H), 7.33 (s, 1H), 7.19-7.21 (m, 1H), 6.83-6.94 (m, 5H), 5.62 (s, 2H), 3.25 (d, J=13.2 Hz, 1H), 3.06 (d, J=13.2 Hz, 1H), 2.71-2.79 (m, 2H), 2.37-2.40 (m, 1H), 1.55 (s, 3H), 1.03 (d, J=6.8 Hz, 3H), 0.92 (d, J=6.8 Hz, 3H). ¹⁹F NMR (376 MHz, DMSO-d₆) δ −116.70, −138.35, −139.77. LC-MS: m/z 598.3 (M+H)⁺.

### Example 7

### (S)-7-{7-[(R)-4-aza-1-indanylamino]-1-thia-4-aza-2-indenyl}-3-[(p-fluorophenyl)methyl]-5-isobutyl-3-methyl-1-oxo-2,4-diaza-6-indancarboxamide (Compound 110)

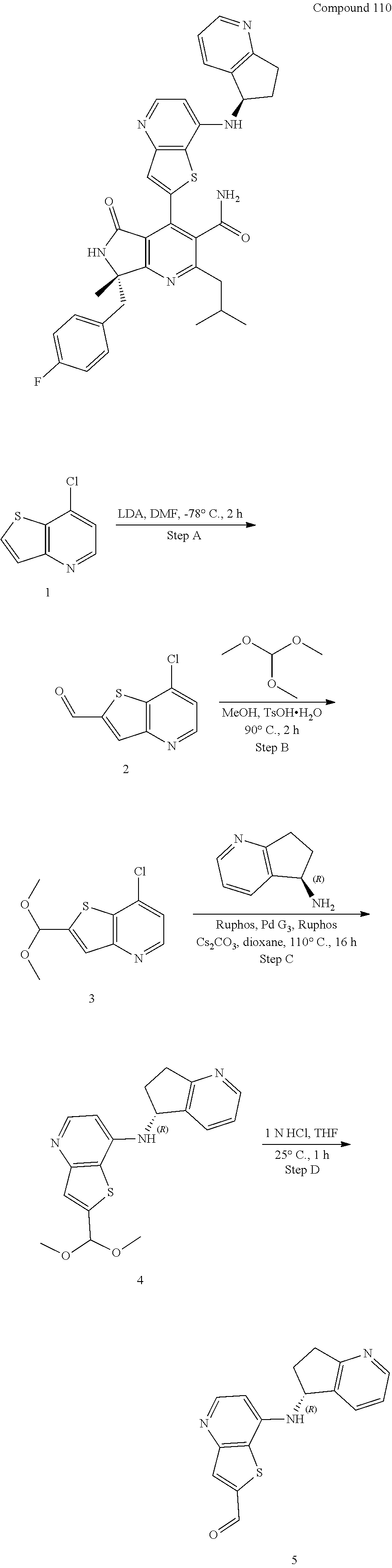

### Step A: 7-chlorothieno[3,2-b]pyridine-2-carbaldehyde

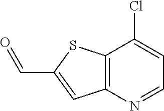 [SMILES: O=Cc1cc2nccc(Cl)c2s1]

To a solution of DIEA (1.248 mL, 8.843 mmol) in THF (2 mL) was added ⁿBuLi (3.301 mL, 8.253 mmol) at −65° C. under Ar. The reaction mixture was stirred at −10° C. for 5 min after addition. Then the mixture was cooled to −65° C., and a solution of 7-chlorothieno[3,2-b]pyridine (1 g, 5.895 mmol) in THF (2 mL) was added at −65° C. slowly. The reaction mixture was stirred at −65° C. for another 1 hour. DMF (2.373 mL, 29.476 mmol) was added. The reaction mixture was allowed to warm to 25° C. and stirred at 25° C. for another 16 hours under Ar. The resulting residue was poured into aq. NH₄Cl (50 mL) and extracted with EA (50 mL×3). The combined organics was washed with brine (50 mL) and dried over anhydrous Na₂SO₄, filtered, and concentrated under vacuum. The resulting residue was purified by silica gel column chromatography (eluting PE/EA=5/1) to afford 7-chlorothieno[3,2-b]pyridine-2-carbaldehyde (1.1 g, 94.41%). LC-MS: m/z 198.2 (M+H)⁺.

### Step B: (7-chlorothieno[3,2-b]pyridin-2-yl)dimethoxymethane

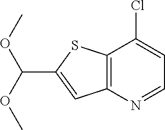

To a solution of 7-chlorothieno[3,2-b]pyridine-2-carbaldehyde (1.101 g, 5.571 mmol) in MeOH (60 mL) were added 4-methylbenzenesulfonic acid oxidane (0.21 g, 1.114 mmol) and trimethoxymethane (3.909 mL, 35.653 mmol). The reaction mixture was stirred at 90° C. for 2 hours under N₂. The resulting mixture was concentrated under vacuum and purified by silica gel column chromatography (eluting PE/EA=5/1) to afford (7-chlorothieno[3,2-b]pyridin-2-yl)dimethoxymethane (1.2 g, 88.39%). LC-MS: m/z 244.0 (M+H)⁺.

### Step C: 1-{[(3,4-difluorophenyl)methyl]oxy}-2,3-dihydro-1H-indene-5-carbaldehyde

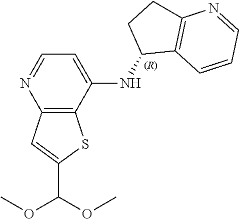

To a solution of (7-chlorothieno[3,2-b]pyridin-2-yl)dimethoxymethane (100 mg, 0.410 mmol) in dioxane (10 mL) were added (5R)-6,7-dihydro-5H-cyclopenta[1,2-b]pyridin-5-amine (149.87 mg, 0.615 mmol), RuPhos Pd G3 (1.07 g, 1.272 mmol), RuPhos (38.30 mg, 0.082 mmol) and Cs₂CO₃ (401.08 mg, 1.231 mmol). The reaction mixture was stirred at 110° C. for 16 hours under Ar. The resulting mixture was concentrated under vacuum and purified by prep-TLC (DCM/MeOH=10/1) to afford (7-{[(5R)-6,7-dihydro-5H-cyclopenta[1,2-b]pyridin-5-yl]amino}thieno[3,2-b]pyridin-2-yl)dimethoxymethane (140 mg, 99.93%). LC-MS: m/z 342.6 (M+H)⁺.

### Step D: 7-{[(5R)-6,7-dihydro-5H-cyclopenta[1,2-b]pyridin-5-yl]amino}thieno[3,2-b]pyridine-2-carbaldehyde

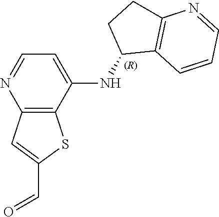

To a solution of (7-{[(5R)-6,7-dihydro-5H-cyclopenta[1,2-b]pyridin-5-yl]amino}thieno[3,2-b]pyridin-2-yl)dimethoxymethane (140 mg, 0.410 mmol) in THF (5 mL) and H₂O (5 mL) was added 1 N HCl (4.100 mL, 4.100 mmol) at 0° C. under N₂. The reaction mixture was stirred at room temperature for 1 hour under N₂. To the reaction mixture was added aq. Na₂CO₃ to pH=8. The resulting residue was poured into H₂O (50 mL) and extracted with EA (50 mL×3). The combined organic phase was washed with brine (50 mL) and dried over anhydrous Na₂SO₄, filtered and concentrated under vacuum. The resulting residue was purified by prep-TLC (eluting DCM/MeOH=10/1) to afford 7-{[(5R)-6,7-dihydro-5H-cyclopenta[1,2-b]pyridin-5-yl]amino}thieno[3,2-b]pyridine-2-carbaldehyde (116 mg, 95.78%). LC-MS: m/z 295.9 (M+H)⁺.

### (S)-7-{7-[(R)-4-aza-1-indanylamino]-1-thia-4-aza-2-indenyl}-3-[(p-fluorophenyl)methyl]-5-isobutyl-3-methyl-1-oxo-2,4-diaza-6-indancarboxamide (Compound 110)

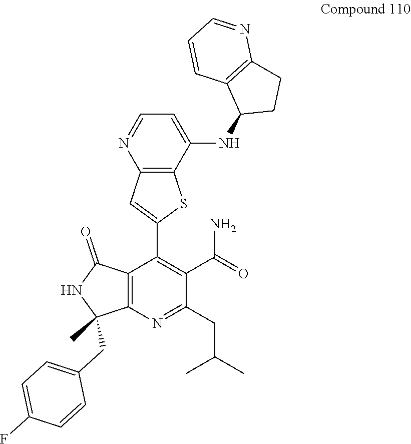 [SMILES: CC(C)Cc1nc2c(c(-c3cc4nccc(N[C@@H]5CCc6ncccc65)c4s3)c1C(N)=O)C(=O)N[C@@]2(C)Cc1ccc(F)cc1]

Compound 110 was then synthesized following the similar route of Example 2, using 7-{[(5R)-6,7-dihydro-5H-cyclopenta[1,2-b]pyridin-5-yl]amino}thieno[3,2-b]pyridine-2-carbaldehyde in step B.

¹H NMR (400 MHz, DMSO-d₆) δ 8.82 (s, 1H), 8.41 (d, J=4.8 Hz, 1H), 8.25 (d, J=5.2 Hz, 1H), 7.84 (s, 1H), 7.62 (d, J=7.6 Hz, 1H), 7.55 (s, 1H), 7.14-7.18 (m, 2H), 7.11 (d, J=8.4 Hz, 1H), 6.86-6.91 (m, 4H), 6.73 (d, J=5.6 Hz, 1H), 5.34-5.40 (m, 1H), 3.24-3.25 (m, 1H), 3.06-3.11 (m, 1H), 3.01-3.05 (m, 1H), 2.91-2.95 (m, 1H), 2.76-2.81 (m, 2H), 2.54-2.63 (m, 1H), 2.36-2.40 (m, 1H), 2.05-2.10 (m, 1H), 1.55 (s, 3H), 1.03 (d, J=6.8 Hz, 3H), 0.93 (d, J=6.8 Hz, 3H). ¹⁹F NMR (376 MHz, DMSO-d₆) δ −116.45. LC-MS: m/z 621.6 (M+H)⁺.

### Example 8

### (1S)-1′-[(3,4-difluorophenyl)methoxy]-1-[(p-fluorophenyl)methyl]-6-isobutyl-1-methyl-3-oxo-2,7-diaza-[4,5′-biindanyl]-5-carboxamide (Compound 111)

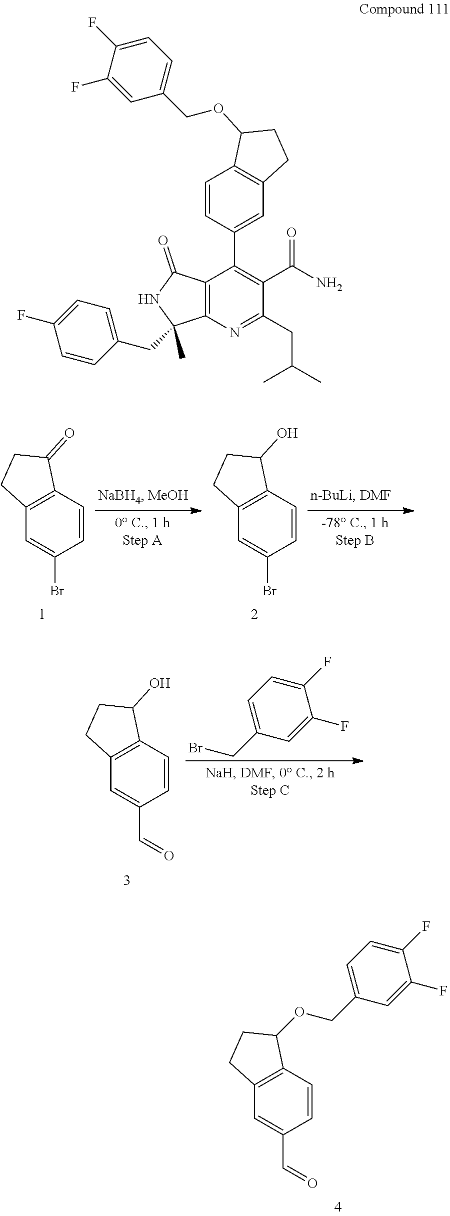

### Step A: 5-bromo-2,3-dihydro-1H-inden-1-ol

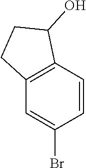 [SMILES: OC1CCc2cc(Br)ccc21]

To a solution of 5-bromo-2,3-dihydro-1H-inden-1-one (5 g, 23.690 mmol) in MeOH (80 mL) was added NaBH₄ (2.69 g, 71.070 mmol). The mixture was stirred at 0° C. for 1 hour under N₂. The reaction mixture was quenched by H₂O (5 ml.). The aqueous phase was concentrated under vacuum. The resulting residue was purified by silica gel column chromatography (eluting PE/EA=8/1) to afford 5-bromo-2,3-dihydro-1H-inden-1-ol (4.64 g, 91.92%).

### Step B: 1-hydroxy-2,3-dihydro-1H-indene-5-carbaldehyde

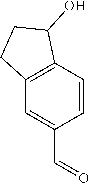 [SMILES: O=Cc1ccc2c(c1)CCC2O]

To a solution of 5-bromo-2,3-dihydro-1H-inden-1-ol (4.64 g, 21.791 mmol) in THF (80 mL) was added ⁿBuLi (40.858 mL, 65.373 mmol) at −78° C. under N₂. The reaction mixture was stirred at −78° C. for 1 hour. DMF (5 mL, 62.115 mmol) was added. The reaction mixture was allowed to warm to 25° C. and stirred at 25° C. for another 1 hour under N₂. The reaction mixture was poured with H₂O (100 mL) and saturated NaCl solution (100 mL), the mixture was extracted with EA (100 mL×2), the combined organic layers was dried over Na₂SO₄, filtered and concentrated under vacuum. The resulting residue was purified by silica gel column chromatography (eluting PE/EA=4/1) to afford 1-hydroxy-2,3-dihydro-1H-indene-5-carbaldehyde (2.00 g, 56.59%). LC-MS: m/z 163.1 (M+H)⁺.

### Step C: 1-{[(3,4-difluorophenyl)methyl]oxy}-2,3-dihydro-1H-indene-5-carbaldehyde

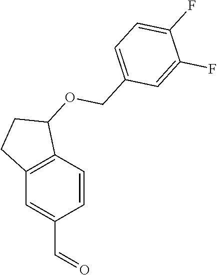 [SMILES: O=Cc1ccc2c(c1)CCC2OCc1ccc(F)c(F)c1]

To a solution of 1-hydroxy-2,3-dihydro-1H-indene-5-carbaldehyde (2 g, 12.331 mmol) in DMF (20 mL) was added NaH (0.59 g, 14.797 mmol) at 0° C. The reaction mixture was stirred at 0° C. for 20 min under N₂, then 4-(bromomethyl)-1,2-difluorobenzene (2.55 g, 12.331 mmol) was added. The reaction mixture was stirred at 25° C. for 2 hours under N₂. The reaction mixture was poured into H₂O (50 mL) and extracted with EA (50 mL×3). The combined organics was washed with brine (100 mL) and dried over Na₂SO₄, filtered and concentrated under vacuum. The resulting residue was purified by silica gel column chromatography (eluting PE/EA=10/1) to afford 1-{[(3,4-difluorophenyl)methyl]oxy}-2,3-dihydro-1H-indene-5-carbaldehyde (282 mg, 6.74%). LC-MS: m/z 289.1 (M+H)⁺.

### (1S)-1′-[(3,4-difluorophenyl)methoxy]-1-[(p-fluorophenyl)methyl]-6-isobutyl-1-methyl-3-oxo-2,7-diaza-[4,5′-biindanyl]-5-carboxamide (Compound 111)

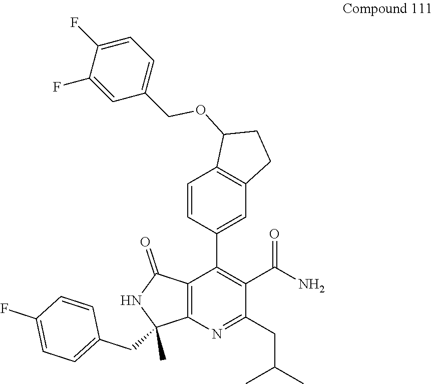 [SMILES: CC(C)Cc1nc2c(c(-c3ccc4c(c3)CCC4OCc3ccc(F)c(F)c3)c1C(N)=O)C(=O)N[C@@]2(C)Cc1ccc(F)cc1]

Compound 111 was then synthesized following the similar route of Example 2, using 1-{[(3,4-difluorophenyl)methyl]oxy}-2,3-dihydro-1H-indene-5-carbaldehyde in step B.

¹H NMR (400 MHz, DMSO-d₆) δ 8.64 (s, 1H), 7.65 (s, 1H), 7.37-7.45 (m, 3H), 7.32 (d, J=8.0 Hz, 1H), 7.22-7.25 (m, 1H), 6.83-7.00 (m, 6H), 4.99 (t, J=6.0 Hz, 1H), 4.61 (q, J=12.8 Hz, 2H), 3.24 (d, J=13.2 Hz, 1H), 3.05 (d, J=13.2 Hz, 1H), 2.91-2.95 (m, 1H), 2.71-2.82 (m, 3H), 2.34-2.41 (m, 2H), 1.99-2.03 (m, 1H), 1.55 (s, 3H), 1.02 (d, J=6.4 Hz, 3H), 0.92 (d, J=6.4 Hz, 3H). ¹⁹F NMR (376 MHz, DMSO-d₆) δ −116.76, −139.00, −140.85. LC-MS: m/z 614.2 (M+H)⁺.

### Example 9

### (1S,1′S)-1′-[(3,4-difluorophenyl)methoxy]-1-[(p-fluorophenyl)methyl]-6-isobutyl-1-methyl-3-oxo-2,7-diaza-[4,5′-biindanyl]-5-carboxamide, and (1S,1′R)-1′-[(3,4-difluorophenyl)methoxy]-1-[(p-fluorophenyl)methyl]-6-isobutyl-1-methyl-3-oxo-2,7-diaza-[4,5′-biindanyl]-5-carboxamide (Compound 112 and Compound 113)

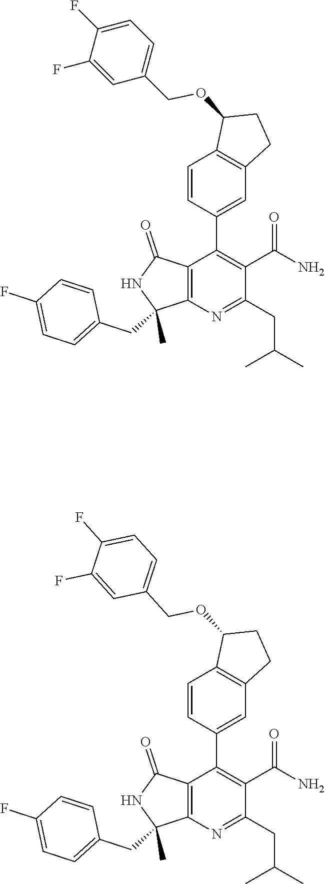 [SMILES: CC(C)Cc1nc2c(c(-c3ccc4c(c3)CC[C@@H]4OCc3ccc(F)c(F)c3)c1C(N)=O)C(=O)N[C@@]2(C)Cc1ccc(F)cc1 ; CC(C)Cc1nc2c(c(-c3ccc4c(c3)CC[C@H]4OCc3ccc(F)c(F)c3)c1C(N)=O)C(=O)N[C@@]2(C)Cc1ccc(F)cc1]

(1S)-1′-[(3,4-difluorophenyl)methoxy]-1-[(p-fluorophenyl)methyl]-6-isobutyl-1-methyl-3-oxo-2,7-diaza-[4,5′-biindanyl]-5-carboxamide (55.36 mg, 0.089 mmol) was purified by SFC (system: Waters SFC 150; Column name: DAICEL CHIRALPAK® AD; Column size: 250*30 mm 10 μm; Mobile Phase A: Supercritical CO₂; Mobile Phase B: IPA; Gradient: A/B=62/38; Flow rate: 70 mL/min; Column Temp: RT) to afford (1S,1′S)-1′-[(3,4-difluorophenyl)methoxy]-1-[(p-fluorophenyl)methyl]-6-isobutyl-1-methyl-3-oxo-2,7-diaza-[4,5′-biindanyl]-5-carboxamide and (1S,1′R)-1′-[(3,4-difluorophenyl)methoxy]-1-[(p-fluorophenyl)methyl]-6-isobutyl-1-methyl-3-oxo-2,7-diaza-[4,5′-biindanyl]-5-carboxamide.

Compound 112, enantiomer 1 (24.72 mg, 44.65%): ¹H NMR (400 MHz, DMSO-d₆) δ 8.64 (s, 1H), 7.65 (s, 1H), 7.36-7.45 (m, 3H), 7.32 (d, J=7.6 Hz, 1H), 7.22-7.25 (m, 1H), 6.88-7.00 (m, 4H), 6.82-6.85 (m, 2H), 4.98-5.01 (m, 1H), 4.56-4.65 (m, 2H), 3.24 (d, J=13.2 Hz, 1H), 3.05 (d, J=13.2 Hz, 1H), 2.90-2.96 (m, 1H), 2.67-2.80 (m, 3H), 2.33-2.39 (m, 2H), 1.98-2.02 (m, 1H), 1.55 (s, 3H), 1.02 (d, J=6.8 Hz, 3H), 0.92 (d, J=6.4 Hz, 3H). ¹⁹F NMR (376 MHz, DMSO-d₆) δ −116.75, −139.00, −140.84. LC-MS: m/z 614.1 (M+H)⁺.

Compound 113, enantiomer 2 (20.63 mg, 37.27%): ¹H NMR (400 MHz, DMSO-d₆) δ 8.64 (s, 1H), 7.64 (s, 1H), 7.36-7.45 (m, 3H), 7.32 (d, J=8.0 Hz, 1H), 7.21-7.25 (m, 1H), 6.88-7.00 (m, 4H), 6.82-6.86 (m, 2H), 4.98-5.00 (m, 1H), 4.56-4.64 (m, 2H), 3.24 (d, J=13.2 Hz, 1H), 3.05 (d, J=13.2 Hz, 1H), 2.91-2.97 (m, 1H), 2.71-2.80 (m, 3H), 2.32-2.39 (m, 2H), 1.98-2.03 (m, 1H), 1.55 (s, 3H), 1.02 (d, J=6.4 Hz, 3H), 0.92 (d, J=6.4 Hz, 3H). ¹⁹F NMR (376 MHz, DMSO-d₆) δ −116.76, −139.00, −140.85. LC-MS: m/z 614.0 (M+H)⁺.

### Example 10

### (S)-7-{5-[N-(R)-1-indanylcarbamoyl]-2-thienyl}-3-[(p-fluorophenyl)methyl]-5-isobutyl-2-methyl-1-oxo-2,4-diaza-6-indancarboxamide (Compound 114)

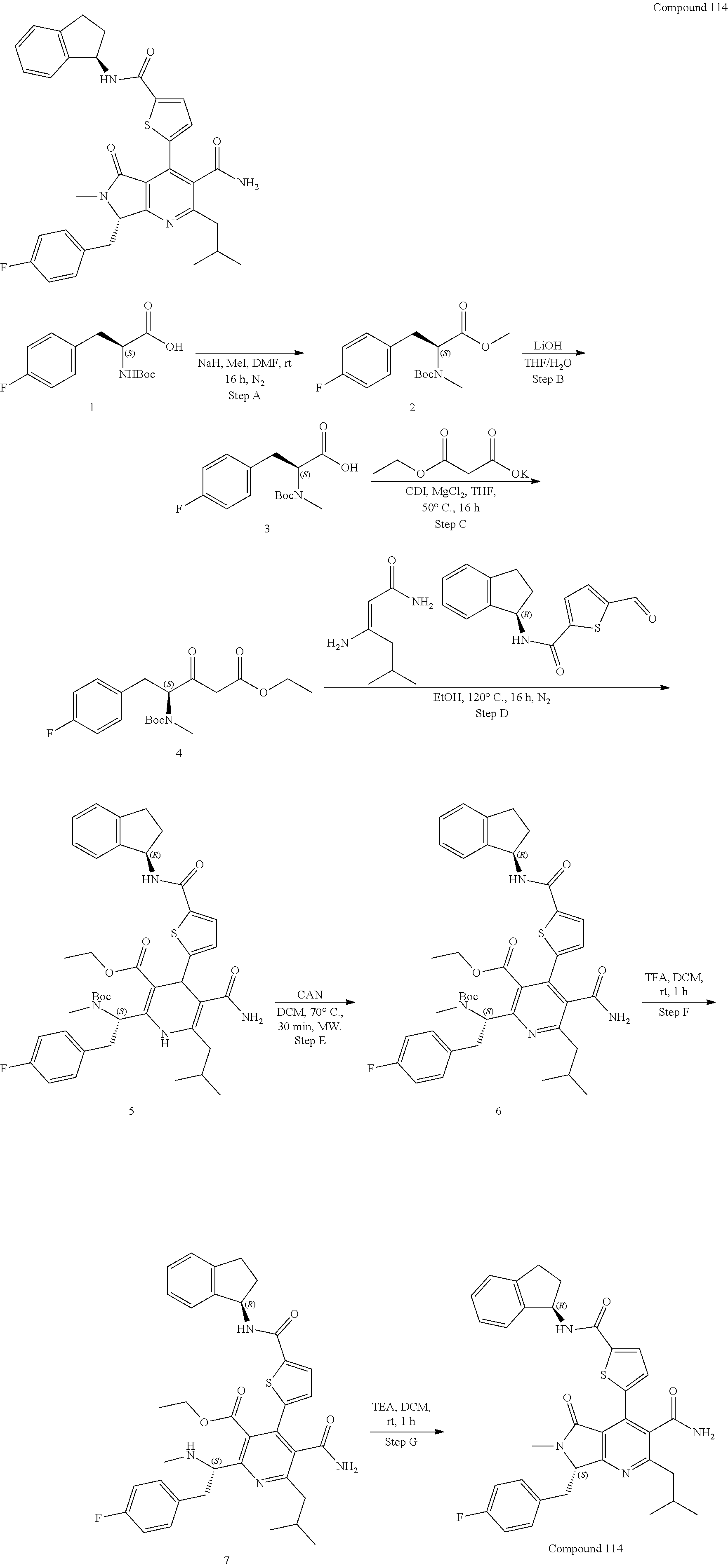

### Step A: methyl(S)-2-((tert-butoxycarbonyl)(methyl)amino)-3-(4-fluorophenyl)propanoate

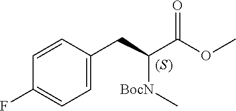

To a solution of (2S)-3-(4-fluorophenyl)-2-({[(2-methylprop-2-yl)oxy]carbonyl}amino)propanoic acid (3.3 g, 11.648 mmol) (3.3 g, 11.64 mmol) in DMF (40 mL) was added NaH (0.34 g, 13.98 mmol) at 0° C., after stirring at room temperature 30 mins, Mel (1.45 ml, 23.30 mmol) was added and the reaction mixture was stirred at room temperature overnight with N₂ protection. The reaction mixture was diluted with EA. The organic layer was separated, washed with brine, and concentrated in vacuum. The residue was purified by using silica gel column chromatography eluting with PE:EA=5:1 to afford methyl(S)-2-((tert-butoxycarbonyl)(methyl)amino)-3-(4-fluorophenyl)propanoate (3.4 g, 93.75%). LC-MS: m/z 212.1 (M+H-Boc)⁺.

### Step B: (2S)-2-(2,2-dimethyl-4-oxo-5-aza-3-oxahex-5-yl)-3-(4-fluorophenyl) propanoic acid

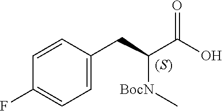

To a solution of methyl(2S)-2-(2,2-dimethyl-4-oxo-5-aza-3-oxahex-5-yl)-3-(4-fluorophenyl) propanoate (1000.00 mg, 3.212 mmol) in THF (8 mL) and H₂O (2 mL) was added LiOH (369 mg, 16.06 mmol) and the reaction mixture was stirred at room temperature 2 hours. The reaction mixture was diluted with H₂O (50 mL), adjusted pH to 2-3 by addition of 0.5 N HCl, then extracted with EA (30 mL×3). the organic layer was separated, washed with brine, and concentrated in vacuum. The residue was purified by using silica gel column chromatography eluting with DCM:MeOH=20:1 to afford (2S)-2-(2,2-dimethyl-4-oxo-5-aza-3-oxahex-5-yl)-3-(4-fluorophenyl) propanoic acid (800 mg, 83.77%). LC-MS: m/z 198.1 (M+H-Boc)⁺.

### Step C: ethyl(S)-4-((tert-butoxycarbonyl)(methyl)amino)-5-(4-fluorophenyl)-3-oxopentanoate

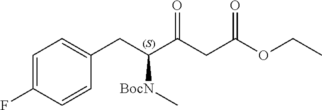

To a solution of (2S)-2-(2,2-dimethyl-4-oxo-5-aza-3-oxahex-5-yl)-3-(4-fluorophenyl) propanoic acid (800 mg, 2.69 mmol) in THF (12 mL) was added CDI (655 mg, 4.04 mmol), after stirring at room temperature 1 hour, MgCl₂ (256 mg, 2.69 mmol) and [(3-ethoxy-1,3-dioxopropyl)oxy]potassium (458 mg, 2.69 mmol) were added, and the reaction mixture was stirred at 50° C. overnight. The reaction mixture was diluted with EA. The organic layer was separated, washed with brine, and concentrated in vacuum. The residue was purified by silica gel column chromatography eluting with PE:EA=5:1 to afford ethyl (S)-4-((tert-butoxycarbonyl)(methyl)amino)-5-(4-fluorophenyl)-3-oxopentanoate (440 mg, 44.51%). LC-MS: m/z 268.1 (M+H)⁺.

### Step D: ethyl 2-((S)-1-((tert-butoxycarbonyl)(methyl)amino)-2-(4-fluorophenyl)ethyl)-5-carbamoyl-4-(5-(((R)-2,3-dihydro-1H-inden-1-yl)carbamoyl)thiophen-2-yl)-6-isobutyl-1,4-dihydropyridine-3-carboxylate

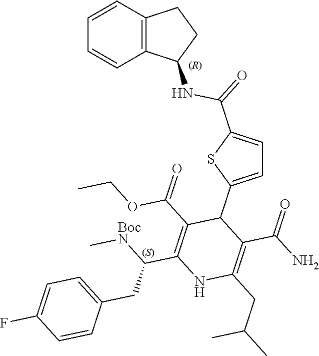

To a solution of ethyl(S)-4-((tert-butoxycarbonyl)(methyl)amino)-5-(4-fluorophenyl)-3-oxopentanoate (271 mg, 0.74 mmol) in EtOH (4 mL) were added (2E)-3-amino-5-methylhex-2-enamide (104.82 mg, 0.737 mmol), N-[(1R)-2,3-dihydro-1H-indenyl]-5-formylthiophene-2-carboxamide (200 mg, 0.74 mmol) and the reaction mixture was stirred at 120° C. overnight with N₂ protection in a sealed tube. The residue was purified by silica gel column chromatography eluting with PE:EA=1:1 to afford ethyl 2-((S)-1-((tert-butoxycarbonyl)(methyl)amino)-2-(4-fluorophenyl)ethyl)-5-carbamoyl-4-(5-(((R)-2,3-dihydro-1H-inden-1-yl)carbamoyl)thiophen-2-yl)-6-isobutyl-1,4-dihydropyridine-3-carboxylate (112 mg, 20.79%). LC-MS: m/z 745.3 (M+H)⁺.

### Step E: ethyl 2-((S)-1-((tert-butoxycarbonyl)(methyl)amino)-2-(4-fluorophenyl)ethyl)-5-carbamoyl-4-(5-(((R)-2,3-dihydro-1H-inden-1-yl)carbamoyl)thiophen-2-yl)-6-isobutylnicotinate

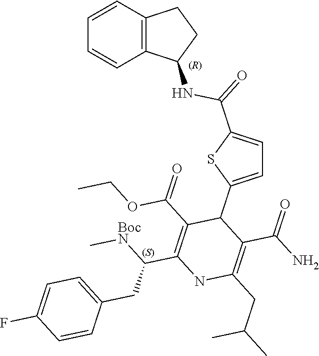

To a solution of ethyl 2-((S)-1-((tert-butoxycarbonyl)(methyl)amino)-2-(4-fluorophenyl)ethyl)-5-carbamoyl-4-(5-(((R)-2,3-dihydro-1H-inden-1-yl)carbamoyl)thiophen-2-yl)-6-isobutyl-1,4-dihydropyridine-3-carboxylate (110 mg, 0.15 mmol) in DCM (15 mL) was added ceric ammonium nitrate (243 mg, 0.44 mmol) and the reaction mixture was stirred at 70° C. for 10 mins under microwave. The reaction mixture was filtered and concentrated in vacuo to afford ethyl 2-((S)-1-((tert-butoxycarbonyl)(methyl)amino)-2-(4-fluorophenyl)ethyl)-5-carbamoyl-4-(5-(((R)-2,3-dihydro-1H-inden-1-yl)carbamoyl)thiophen-2-yl)-6-isobutylnicotinate (75 mg, 68.37%) which was used in the next step without further purification. LC-MS: m/z 743.2 (M+H)⁺.

### Step F: ethyl 5-carbamoyl-4-(5-(((R)-2,3-dihydro-1H-inden-1-yl)carbamoyl)thiophen-2-yl)-2-((S)-2-(4-fluorophenyl)-1-(methylamino)ethyl)-6-isobutylnicotinate

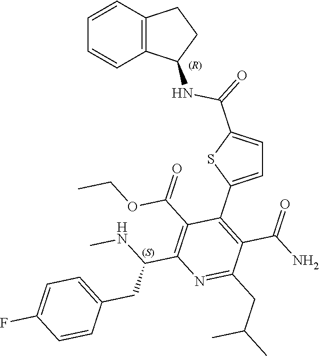

To a solution of ethyl 2-((S)-1-((tert-butoxycarbonyl)(methyl)amino)-2-(4-fluorophenyl)ethyl)-5-carbamoyl-4-(5-(((R)-2,3-dihydro-1H-inden-1-yl)carbamoyl)thiophen-2-yl)-6-isobutylnicotinate (85 mg, 0.12 mmol) in DCM (2.5 mL) was added TFA (0.5 mL) and the reaction mixture was stirred at room temperature for 1.5 hours. The reaction mixture was diluted with DCM and sat. Na₂CO₃. The organic layer was separated, washed with brine and concentrated in vacuo to afford ethyl 5-carbamoyl-4-(5-(((R)-2,3-dihydro-1H-inden-1-yl)carbamoyl)thiophen-2-yl)-2-((S)-2-(4-fluorophenyl)-1-(methylamino)ethyl)-6-isobutylnicotinate (72 mg, 97.90%). LC-MS: m/z 643.4 (M+H)⁺.

### Step G: (S)-7-{5-[N-(R)-1-indanylcarbamoyl]-2-thienyl}-3-[(p-fluorophenyl)methyl]-5-isobutyl-2-methyl-1-oxo-2,4-diaza-6-indancarboxamide (Compound 114)

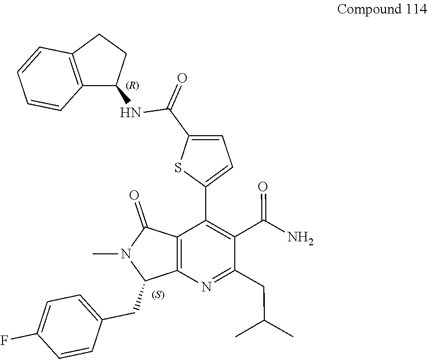

To a solution of ethyl 5-carbamoyl-4-(5-(((R)-2,3-dihydro-1H-inden-1-yl)carbamoyl)thiophen-2-yl)-2-((S)-2-(4-fluorophenyl)-1-(methylamino)ethyl)-6-isobutylnicotinate (50 mg, 0.078 mmol) in DCM (1.5 mL) was added TEA (0.5 mL) and the reaction mixture was stirred at room temperature for 2 hours. The reaction mixture was concentrated and purified by Prep-HPLC with 0.1% FA to afford (S)-7-{5-[N-(R)-1-indanylcarbamoyl]-2-thienyl}-3-[(p-fluorophenyl)methyl]-5-isobutyl-2-methyl-1-oxo-2,4-diaza-6-indancarboxamide (2 mg, 4.31%).

¹H NMR (400 MHz, DMSO-d₆) δ 8.85 (d, J=8.4 Hz, 1H), 7.83 (s, 1H), 7.73 (d, J=4.0 Hz, 1H), 7.58 (s, 1H), 7.19-7.29 (m, 4H), 7.01 (d, J=3.6 Hz, 1H), 6.94-7.02 (m, 4H), 5.48-5.54 (m, 1H), 4.88 (t, J=4.0 Hz, 1H), 3.39-3.42 (m, 2H), 2.96-3.03 (m, 4H), 2.82-2.90 (m, 1H), 2.74-2.76 (m, 2H), 2.44-2.46 (m, 1H), 2.33-2.40 (m, 1H), 1.92-2.02 (m, 1H), 0.98 (d, J=6.4 Hz, 3H), 0.90 (d, J=6.4 Hz, 3H). ¹⁹F NMR (376 MHz, DMSO-d₆) δ −116.31. LC-MS: m/z 597.3 (M+H)⁺.

### Example 11

### (S)-4-{5-[N-(R)-1-indanylcarbamoyl]-2-thienyl}-7-[(p-fluorophenyl)methyl]-2-isobutyl-6-methyl-5-oxo-5,6,7,8-tetrahydro-1,6-diaza-3-naphthamide (Compound 115)

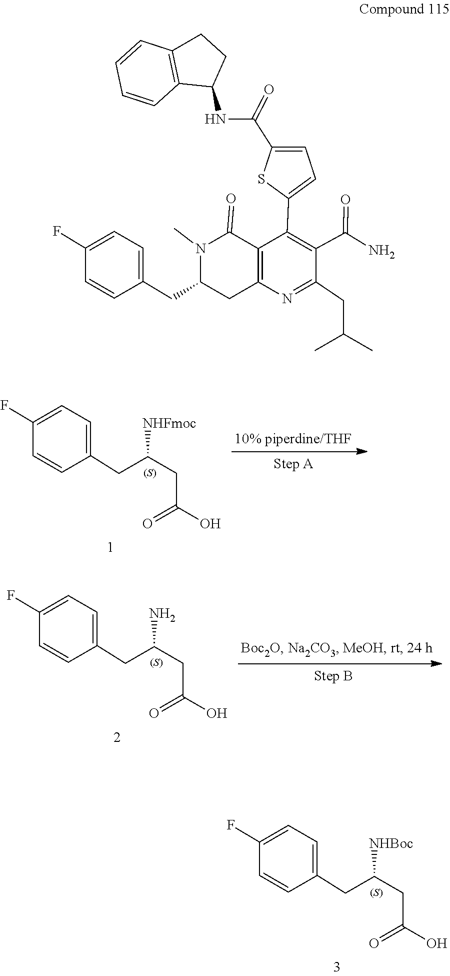

### Step A: (3S)-3-amino-4-(4-fluorophenyl) butanoic acid

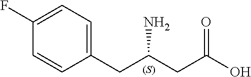

To a solution of (3S)-4-(4-fluorophenyl)-3-({[(9H-fluoren-9-ylmethyl)oxy]carbonyl}amino) butanoic acid (1 g, 2.38 mmol) in DMF (10 mL) was added piperidine (1 mL) and the reaction mixture was stirred at room temperature for 30 minutes. The reaction mixture was diluted with EA and brine. The water layer was concentrated in vacuum to afford (3S)-3-amino-4-(4-fluorophenyl) butanoic acid (1 g crude) which was used directly in the next step without further purification. LC-MS: m/z 198.0 (M+H)⁺.

### Step B: (3S)-4-(4-fluorophenyl)-3-({[(2-methylprop-2-yl)oxy]carbonyl}amino)butanoic acid

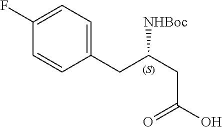

To a solution of (3S)-3-amino-4-(4-fluorophenyl) butanoic acid (1 g, crude) in MeOH (20 mL) and H₂O (10 mL) were added (Boc)₂O (5 mL, 5.071 mmol) and Na₂CO₃ (2.69 g, 25.35 mmol), the reaction mixture was stirred at room temperature for 24 hours. The reaction mixture was adjusted to pH=5-6 with 0.5 N HCl, and then extracted with EA (50 mL×2), dried, concentrated in vacuum to afford (3S)-4-(4-fluorophenyl)-3-({[(2-methylprop-2-yl)oxy]carbonyl}amino)butanoic acid (620 mg, 41.12%). LC-MS: m/z 241.9 (M+H-^{t}Bu)⁺.

### (S)-4-{5-[N-(R)-1-indanylcarbamoyl]-2-thienyl}-7-[(p-fluorophenyl)methyl]-2-isobutyl-6-methyl-5-oxo-5,6,7,8-tetrahydro-1,6-diaza-3-naphthamide (Compound 115)

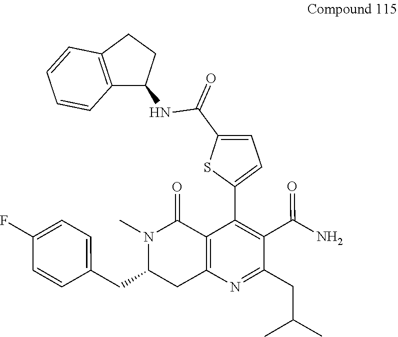 [SMILES: CC(C)Cc1nc2c(c(-c3ccc(C(=O)N[C@@H]4CCc5ccccc54)s3)c1C(N)=O)C(=O)N(C)[C@@H](Cc1ccc(F)cc1)C2]

Compound 115 was then synthesized following the similar route of Example 10, using (3S)-4-(4-fluorophenyl)-3-({[(2-methylprop-2-yl)oxy]carbonyl}amino)butanoic acid in step A.

¹H NMR (400 MHz, CD₃OD) δ 7.65 (d, J=4.0 Hz, 1H), 7.15-7.31 (m, 6H), 6.99-7.06 (m, 3H), 5.62 (t, J=8.0 Hz, 1H), 3.94-3.99 (m, 1H), 3.47-3.52 (m, 1H), 3.06-3.08 (m, 2H), 2.90-3.02 (m, 2H), 2.84 (s, 3H), 2.75-2.81 (m, 3H), 2.56-2.60 (m, 1H), 2.29-2.36 (m, 1H), 2.00-2.05 (m, 1H), 1.00 (d, J=6.8 Hz, 6H). ¹⁹F NMR (376 MHz, CD₃OD) δ −118.01. LC-MS: m/z 611.2 (M+H)⁺.

### Example 12

### (R)-4-{5-[N-(R)-1-indanylcarbamoyl]-2-thienyl}-7-[(p-fluorophenyl)methyl]-2-isobutyl-5-oxo-5,6,7,8-tetrahydro-1,6-diaza-3-naphthamide (Compound 116)

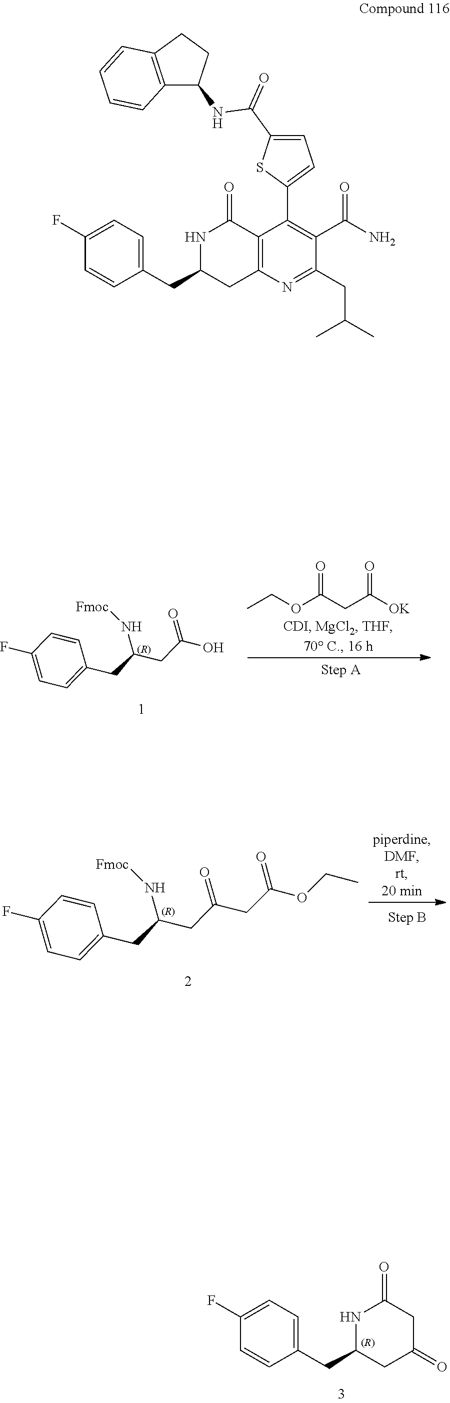

### Step A: ethyl (R)-5-((((9H-fluoren-9-yl) methoxy) carbonyl)amino)-6-(4-fluorophenyl)-3-oxohexanoate

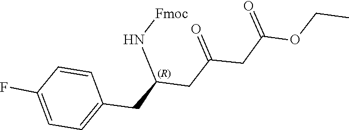

A mixture of (3R)-4-(4-fluorophenyl)-3-({[(9H-fluoren-9-ylmethyl)oxy]carbonyl}amino)butanoic acid (1 g, 2.38 mmol) and CDI (0.58 g, 3.58 mmol) in THF (25 mL) was stirred at room temperature for 1 hour, then [(3-ethoxy-1,3-dioxopropyl)oxy]potassium (1.01 g, 5.96 mmol), and MgCl₂ (0.45 g, 4.77 mmol) were added, and the reaction mixture was stirred at 70° C. overnight. The reaction mixture was diluted with EA and brine. The organic layer was separated, washed with brine, and concentrated in vacuum. The residue was purified by silica gel column chromatography eluting with EA:PE=1:5 to afford ethyl (R)-5-((((9H-fluoren-9-yl) methoxy) carbonyl)amino)-6-(4-fluorophenyl)-3-oxohexanoate (680 mg, 58.26%). LC-MS: m/z 490.2 (M+H)⁺.

### Step B: (R)-6-(4-fluorobenzyl)piperidine-2,4-dione

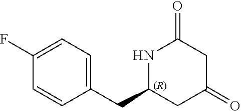

A mixture of ethyl (R)-5-((((9H-fluoren-9-yl) methoxy) carbonyl)amino)-6-(4-fluorophenyl)-3-oxohexanoate (230 mg, 0.47 mmol) in THF (8 mL) and piperidine (0.8 mL) was stirred at room temperature for 30 min. The reaction mixture was diluted with EA and H₂O. The water layer was concentrated in vacuum. The residue was purified by reversed phase column with 0.1% FA to afford (R)-6-(4-fluorobenzyl) piperidine-2,4-dione (40 mg, 38.48%). LC-MS: m/z 222.1 (M+H)⁺.

### (R)-4-{5-[N-(R)-1-indanylcarbamoyl]-2-thienyl}-7-[(p-fluorophenyl)methyl]-2-isobutyl-5-oxo-5,6,7,8-tetrahydro-1,6-diaza-3-naphthamide (Compound 116)

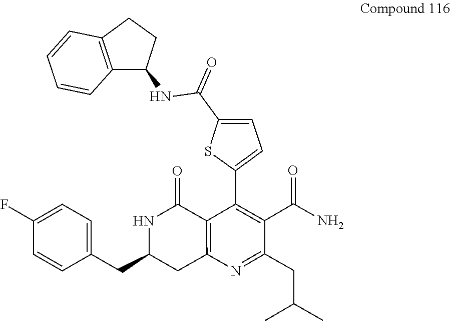 [SMILES: CC(C)Cc1nc2c(c(-c3ccc(C(=O)N[C@@H]4CCc5ccccc54)s3)c1C(N)=O)C(=O)N[C@H](Cc1ccc(F)cc1)C2]

Compound 116 was then synthesized following the similar route of Example 10, using (R)-6-(4-fluorobenzyl) piperidine-2,4-dione in step D. ¹H NMR (400 MHz, CD₃OD) δ 7.62 (d, J=4.0 Hz, 1H), 7.19-7.28 (m, 6H), 7.03-7.07 (m, 2H), 6.98 (d, J=4.0 Hz, 1H), 5.60 (t, J=8.0 Hz, 1H), 3.92-3.95 (m, 1H), 3.17-3.22 (m, 1H), 2.89-3.08 (m, 4H), 2.74-2.84 (m, 3H), 2.55-2.59 (m, 1H), 2.28-2.32 (m, 1H), 1.99-2.04 (m, 1H), 0.98 (dd, J=6.8, 2.0 Hz, 6H). ¹⁹F NMR (376 MHz, CD₃OD) δ −118.26. LC-MS: m/z 597.2 (M+H)⁺.

### (S)-4-{1-[2-(3,4-difluorophenyl)ethyl]-5-indolyl}-7-[(p-fluorophenyl)methyl]-2-isobutyl-5-oxo-5,6,7,8-tetrahydro-1,6-diaza-3-naphthamide (Compound 117)

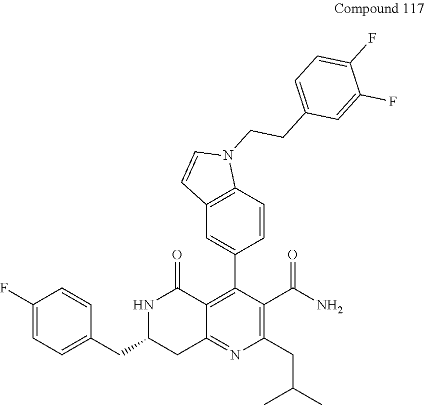 [SMILES: CC(C)Cc1nc2c(c(-c3ccc4c(ccn4CCc4ccc(F)c(F)c4)c3)c1C(N)=O)C(=O)N[C@@H](Cc1ccc(F)cc1)C2]

Compound 117 was synthesized following the similar route of Example 12, using (3S)-4-(4-fluorophenyl)-3-({[(9H-fluoren-9-ylmethyl)oxy]carbonyl}amino)butanoic acid in step A, then following the similar route of Example 10, using(S)-6-(4-fluorobenzyl) piperidine-2,4-dione and 1-[2-(3,4-difluorophenyl)ethyl]indole-5-carbaldehyde in step D. ¹H NMR (400 MHz, DMSO-d₆) δ 7.82 (m, 1H), 7.49-7.42 (m, 4H), 7.37-7.29 (m, 5H), 7.21-7.03 (m, 5H), 6.36 (s, 1H), 4.38 (t, J=7.2 Hz, 2H), 3.83 (m, 1H), 3.09 (t, J=7.6 Hz, 2H), 2.97-2.94 (m, 1H), 2.87-2.85 (m, 1H), 2.74-2.73 (m, 1H), 2.61 (d, J=7.2 Hz, 2H), 2.26-2.21 (m, 1H), 0.90 (dd, J=6.8, 2.0 Hz, 6H). ¹⁹F NMR (376 MHz, DMSO-d₆) δ −116.57, −139.16, −142.16. LC-MS: m/z 611.4 (M+H)⁺.

### (S)-4-{1-[(3,4-difluorophenyl)methoxy]-5-indanyl}-7-[(p-fluorophenyl)methyl]-2-isobutyl-5-oxo-5,6,7,8-tetrahydro-1,6-diaza-3-naphthamide (Compound 118)

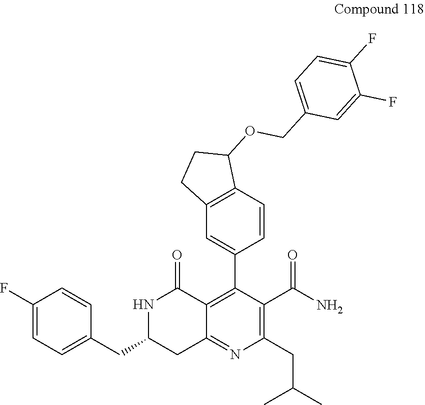 [SMILES: CC(C)Cc1nc2c(c(-c3ccc4c(c3)CCC4OCc3ccc(F)c(F)c3)c1C(N)=O)C(=O)N[C@@H](Cc1ccc(F)cc1)C2]

Compound 118 was synthesized following the similar route of Example 10, using 1-{[(3,4-difluorophenyl)methyl]oxy}-2,3-dihydro-1H-indene-5-carbaldehyde and(S)-6-(4-fluorobenzyl) piperidine-2,4-dione in step D. ¹H NMR (400 MHz, DMSO-d₆) δ 7.86 (m, 1H), 7.55 (m, 1H), 7.46-7.38 (m, 2H), 7.31-7.26 (m, 5H), 7.15 (t, J=8.8 Hz, 2H), 7.08-7.02 (m, 2H), 5.01 (m, 1H), 4.66-4.58 (dd, J=20.4, 12.0 Hz, 2H), 3.84-3.81 (m, 1H), 2.97-2.81 (m, 4H), 2.75-2.67 (m, 2H), 2.61 (d, J=6.8 Hz, 2H), 2.41-2.32 (m, 1H), 2.26-2.19 (m, 1H), 2.08-1.98 (m, 1H), 0.89-0.87 (dd, J=6.4, 1.6 Hz, 6H). ¹⁹F NMR (376 MHz, DMSO-d₆) δ −118.26. LC-MS: m/z 614.4 (M+H)⁺.

### (S)-7-[(p-fluorophenyl)methyl]-2-isobutyl-4-(7-{[(m-methoxyphenyl)methyl]amino}-1-thia-6-aza-2-indenyl)-5-oxo-5,6,7,8-tetrahydro-1,6-diaza-3-naphthamide (Compound 119)

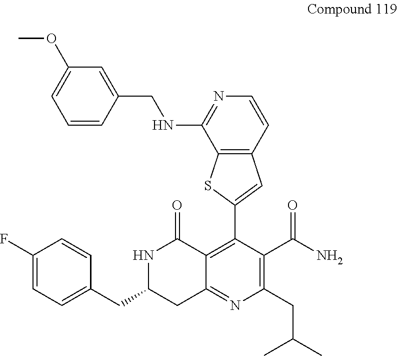 [SMILES: COc1cccc(CNc2nccc3cc(-c4c(C(N)=O)c(CC(C)C)nc5c4C(=O)N[C@@H](Cc4ccc(F)cc4)C5)sc23)c1]

Compound 119 was synthesized following the similar route of Example 12, using (3S)-4-(4-fluorophenyl)-3-({[(9H-fluoren-9-ylmethyl)oxy]carbonyl}amino)butanoic acid in step A, then following the similar route of Example 1, using(S)-4-(7-chlorothieno[2,3-c]pyridin-2-yl)-7-(4-fluorobenzyl)-2-isobutyl-5-oxo-5,6,7,8-tetrahydro-1,6-naphthyridine-3-carboxamide and (3-methoxyphenyl) methanamine in step F. ¹H NMR (400 MHz, DMSO-d₆) δ 8.09 (d, J=2.0 Hz, 1H), 7.86 (d, J=5.6 Hz, 1H), 7.76 (s, 1H), 7.48 (s, 1H), 7.28-7.34 (m, 3H), 7.11-7.25 (m, 4H), 6.99 (d, J=5.6 Hz, 1H), 6.90-6.96 (m, 2H), 6.77 (dd, J=8.4, 1.6 Hz, 1H), 4.60-4.73 (m, 2H), 3.85-3.93 (m, 1H), 3.71 (s, 3H), 2.87-3.02 (m, 3H), 2.70-2.79 (m, 1H), 2.61-2.66 (m, 2H), 2.19-2.30 (m, 1H), 0.90 (dd, J=6.8, 1.2 Hz, 6H). ¹⁹F NMR (376 MHz, DMSO-d₆) δ −116.48. LC-MS: m/z 624.2 (M+H)⁺.

### (S)-7-[(p-fluorophenyl)methyl]-4-(7-{[(5-fluoro-3-pyridyl)methyl]amino}-1-thia-6-aza-2-indenyl)-2-isobutyl-5-oxo-5,6,7,8-tetrahydro-1,6-diaza-3-naphthamide (Compound 120)

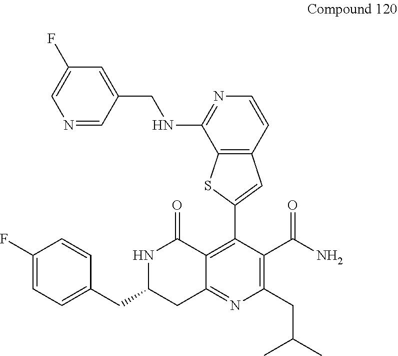 [SMILES: CC(C)Cc1nc2c(c(-c3cc4ccnc(NCc5cncc(F)c5)c4s3)c1C(N)=O)C(=O)N[C@@H](Cc1ccc(F)cc1)C2]

Compound 120 was synthesized following the similar route of Example 12, using (3S)-4-(4-fluorophenyl)-3-({[(9H-fluoren-9-ylmethyl)oxy]carbonyl}amino)butanoic acid in step A, then following the similar route of Example 1, using(S)-4-(7-chlorothieno[2,3-c]pyridin-2-yl)-7-(4-fluorobenzyl)-2-isobutyl-5-oxo-5,6,7,8-tetrahydro-1,6-naphthyridine-3-carboxamide and (5-fluoropyridin-3-yl)methanamine in step F. ¹H NMR (400 MHz, DMSO-d₆) δ 8.48 (s, 1H), 8.42 (d, J=2.8 Hz, 1H), 8.10 (d, J=2.8 Hz, 1H), 7.88 (d, J=5.6 Hz, 1H), 7.76 (s, 1H), 7.62-7.68 (m, 1H), 7.48 (s, 1H), 7.42 (t, J=5.6 Hz, 1H), 7.26-7.33 (m, 2H), 7.12-7.21 (m, 3H), 7.04 (d, J=5.6 Hz, 1H), 4.73 (d, J=6.4 Hz, 2H), 3.78-3.90 (m, 1H), 2.85-3.03 (m, 3H), 2.70-2.80 (m, 1H), 2.62-2.70 (m, 2H), 2.16-2.30 (m, 1H), 0.91 (dd, J=6.8, 1.6 Hz, 6H). ¹⁹F NMR (376 MHz, DMSO-d₆) δ −116.47, −128.08. LC-MS: m/z 613.3 (M+H)⁺.

### Example 13

### (S)-4-{7-[(R)-4-aza-1-indanylamino]-1-thia-6-aza-2-indenyl}-7-[(p-fluorophenyl)methyl]-2-isobutyl-7-methyl-5-oxo-5,6,7,8-tetrahydro-1,6-diaza-3-naphthamide (Compound 121)

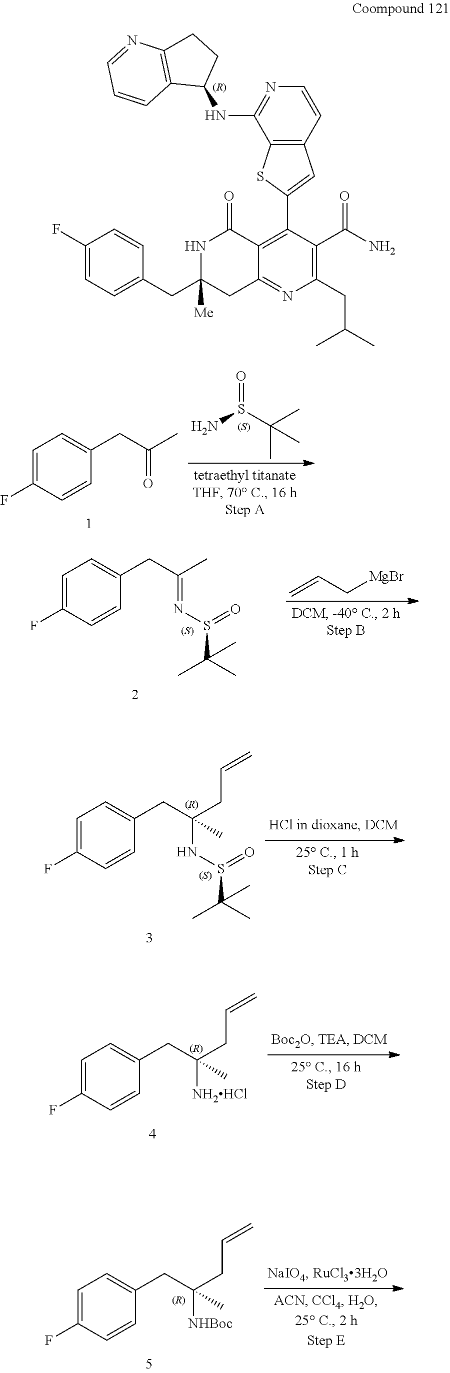

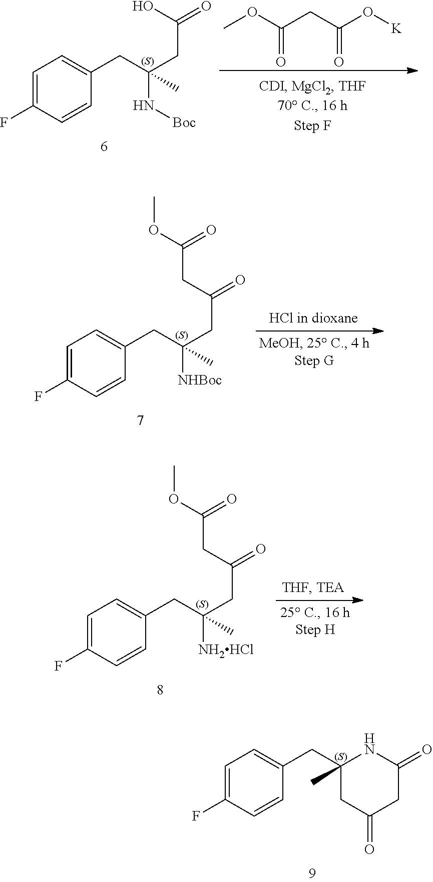

### Step A: (S,E)-N-(1-(4-fluorophenyl) propan-2-ylidene)-2-methylpropane-2-sulfinamide

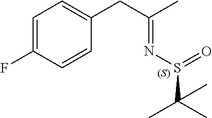

To a solution of 1-(4-fluorophenyl) propan-2-one (1.0 g, 6.572 mmol) and(S)-(2-methylprop-2-yl)(oxo)-λ4-sulfanamine (0.88 g, 7.229 mmol) in THF (30 mL) was added tetraethyl titanate (3.00 g, 13.143 mmol). The reaction mixture was stirred at 70° C. overnight under N₂. To the reaction mixture was added water (50 mL) and EA (50 mL), the mixture was filtered and the filtrate was concentrated under vacuum. The resulting residue was purified by silica gel chromatography (eluting PE/EA=2/1) to afford (S,E)-N-(1-(4-fluorophenyl) propan-2-ylidene)-2-methylpropane-2-sulfinamide (1.46 g, 87.01%) which was used directly in the next step without further purification. LC-MS: m/z 256.1 (M+H)⁺.

### Step B: (S)-N-((R)-1-(4-fluorophenyl)-2-methylpent-4-en-2-yl) propane-2-sulfinamide

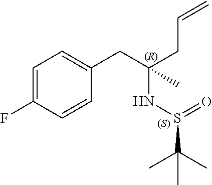

To a solution of (S,E)-N-(1-(4-fluorophenyl) propan-2-ylidene)-2-methylpropane-2-sulfinamide (4.5 g, 17.623 mmol) in DCM (60 mL) was added bromo (prop-2-enyl) magnesium (61.680 mL, 61.680 mmol) at −40° C. under N₂. The reaction mixture was stirred at −40° C. for 2 hours. The reaction mixture was quenched by aq. NH₄Cl (60 mL), the mixture was extracted with DCM (180 mL), the combined organic layers were concentrated under vacuum. The resulting residue was purified by silica gel chromatography (eluting with PE/EA=1/1) to afford (S)-N-((R)-1-(4-fluorophenyl)-2-methylpent-4-en-2-yl) propane-2-sulfinamide (3.5 g, 66.77%). LC-MS: m/z 298.2 (M+H)⁺.

### Step C: (R)-1-(4-fluorophenyl)-2-methylpent-4-en-2-amine, Hydrochloride Salt

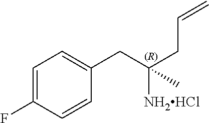

To a solution of(S)-N-((R)-1-(4-fluorophenyl)-2-methylpent-4-en-2-yl)propane-2-sulfinamide (3.4 g, 11.431 mmol) in DCM (30 mL) was added 1,4-dioxane/HCl (6.12 mL) at 25° C. under N₂. The reaction mixture was stirred at 25° C. for 1 hour. The reaction mixture was concentrated under vacuum to afford (R)-1-(4-fluorophenyl)-2-methylpent-4-en-2-amine, hydrochloride salt (2.1 g, 95.02%) which was used directly in the next step without further purification. LC-MS: m/z 194.2 (M+H-HCl)⁺.

### Step D: tert-butyl (R)-(1-(4-fluorophenyl)-2-methylpent-4-en-2-yl)carbamate

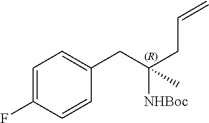

To a solution of (R)-1-(4-fluorophenyl)-2-methylpent-4-en-2-amine, hydrochloride salt (2.1 g, 10.866 mmol) in DCM (30 mL) was added TEA (0.728 mL, 4.657 mmol) and (2-methylprop-2-yl)oxidanecarboxylic anhydride (3.748 mL, 16.298 mmol) at 25° C. under N₂. The reaction mixture was stirred 25° C. for 2 hours. The reaction mixture was quenched by aq. NH₄Cl (30 mL), extracted with DCM (30 mL), and concentrated under vacuum. The resulting residue was purified by TLC (PE/EA=5/1) to afford tert-butyl (R)-(1-(4-fluorophenyl)-2-methylpent-4-en-2-yl)carbamate (1.96 g, 61.49%). LC-MS: m/z 310.0 (M+H)⁺.

### Step E: (S)-3-((tert-butoxycarbonyl)amino)-4-(4-fluorophenyl)-3-methylbutanoic acid

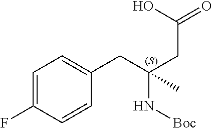

To a solution of tert-butyl (R)-(1-(4-fluorophenyl)-2-methylpent-4-en-2-yl)carbamate (1.96 g, 6.681 mmol) in ACN (20 mL) were added tetrachloromethane (20 mL), H₂O (30 mL), NaIO₄ (5.72 g, 26.723 mmol) and tris(oxidane) trichloroλ-3-ruthenium (III) (0.09 g, 0.334 mmol), the reaction mixture was stirred at 25° C. for 2 hours under N₂. The mixture was concentrated under vacuum and purified by C18 (eluting with 50% of ACN in water, 0.1% FA) to afford (S)-3-((tert-butoxycarbonyl)amino)-4-(4-fluorophenyl)-3-methylbutanoic acid (500 mg, 24.04%). LC-MS: m/z 212.0 (M+H-BoC)⁺.

### Step F: methyl(S)-5-((tert-butoxycarbonyl)amino)-6-(4-fluorophenyl)-5-methyl-3-oxohexanoate

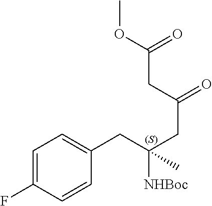

To a solution of(S)-3-((tert-butoxycarbonyl)amino)-4-(4-fluorophenyl)-3-methylbutanoic acid (1.2 g, 3.854 mmol) in THF (40 mL) was added CDI (1.25 g, 7.708 mmol), the mixture was stirred at 25° C. for 2 hours under N₂ protection. Then [(3-methoxy-1,3-dioxopropyl)oxy]potassium (1.20 g, 7.708 mmol) was added, the reaction mixture was stirred for 10 min, then magnesium chloride (0.73 g, 7.708 mmol) was added. The reaction mixture was stirred at 70° C. for another 24 hours under N₂. The reaction mixture was quenched by aq. NaHSO₄ (20 mL), extracted with EA (80 mL), the organic layer was washed by brine (20 mL), dried over anhydrous Na₂SO₄, filtered and concentrated under vacuum. The resulting residue was purified by prep-HPLC (50% of ACN in water, 0.1% FA) to afford methyl(S)-5-((tert-butoxycarbonyl)amino)-6-(4-fluorophenyl)-5-methyl-3-oxohexanoate (700 mg, 49.43%). LC-MS: m/z 268.2 (M+H-BoC)⁺.

### Step G: (S)-5-amino-6-(4-fluorophenyl)-5-methyl-3-oxohexanoate, Hydrochloride Salt

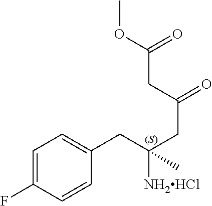

To a solution of methyl(S)-5-((tert-butoxycarbonyl)amino)-6-(4-fluorophenyl)-5-methyl-3-oxohexanoate (300 mg, 0.817 mmol) in MeOH (6 mL) was added 1,4-dioxane/HCl (1.0 mL) at 25° C. under N₂. The reaction mixture was stirred at 25° C. for 1 hour. The mixture was concentrated under vacuum to afford (S)-5-amino-6-(4-fluorophenyl)-5-methyl-3-oxohexanoate, hydrochloride salt (218 mg, 0.816 mmol) which was used directly in the next step without further purification. LC-MS: m/z 268.4 (M+H-HCl)⁺.

### Step H: (S)-6-(4-fluorobenzyl)-6-methylpiperidine-2,4-dione

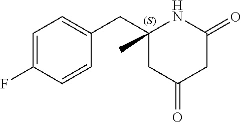

To a solution of(S)-5-amino-6-(4-fluorophenyl)-5-methyl-3-oxohexanoate, hydrochloride salt (218 mg, 0.816 mmol) in THF (5 mL) was added TEA (1.134 mL, 8.156 mmol) at 25° C. under N₂. The reaction mixture was stirred at 25° C. for 1 hour. The mixture was concentrated under vacuum and purified by prep-HPLC (35% of ACN in water, 0.1% FA) to afford (S)-6-(4-fluorobenzyl)-6-methylpiperidine-2,4-dione (120 mg, 62.54%). LC-MS: m/z 236.2 (M+H)⁺.

### (S)-4-{7-[(R)-4-aza-1-indanylamino]-1-thia-6-aza-2-indenyl}-7-[(p-fluorophenyl)methyl]-2-isobutyl-7-methyl-5-oxo-5,6,7,8-tetrahydro-1,6-diaza-3-naphthamide (Compound 121)

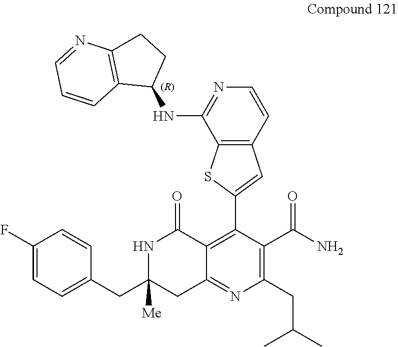

Compound 121 was then synthesized following the similar route of Example 10, using (R)-7-((6,7-dihydro-5H-cyclopenta[b]pyridin-5-yl)amino) thieno[2,3-c]pyridine-2-carbaldehyde and (3S)-5-(4-fluorophenyl)-3-({[(2-methylprop-2-yl)oxy]carbonyl}amino) pentanoic acid in step D.

¹H NMR (400 MHz, DMSO-d₆) δ 8.37 (d, J=4.0 Hz, 1H), 8.00 (s, 1H), 7.95 (d, J=5.6 Hz, 1H), 7.72 (s, 1H), 7.62 (d, J=8.0 Hz, 1H), 7.46 (s, 1H), 7.09-7.19 (m, 7H), 7.06 (d, J=5.6 Hz, 1H), 5.89-5.96 (m, 1H), 2.93-3.07 (m, 4H), 2.80 (d, J=4.0 Hz, 2H), 2.65-2.67 (m, 2H), 2.54-2.58 (m, 1H), 2.23-2.32 (m, 1H), 2.08-2.16 (m, 1H), 1.21 (s, 3H), 0.94 (d, J=6.8 Hz, 3H), 0.90 (d, J=6.8 Hz, 3H). ¹⁹F NMR (376 MHz, DMSO-d₆) δ −116.42. LC-MS: m/z 635.4 (M+H)⁺.

### Example 14

### (S)-4-{7-[(R)-4-aza-1-indanylamino]-1-thia-6-aza-2-indenyl}-7-[2-(p-fluorophenyl)ethyl]-2-isobutyl-5-oxo-5,6,7,8-tetrahydro-1,6-diaza-3-naphthamide (Compound 122)

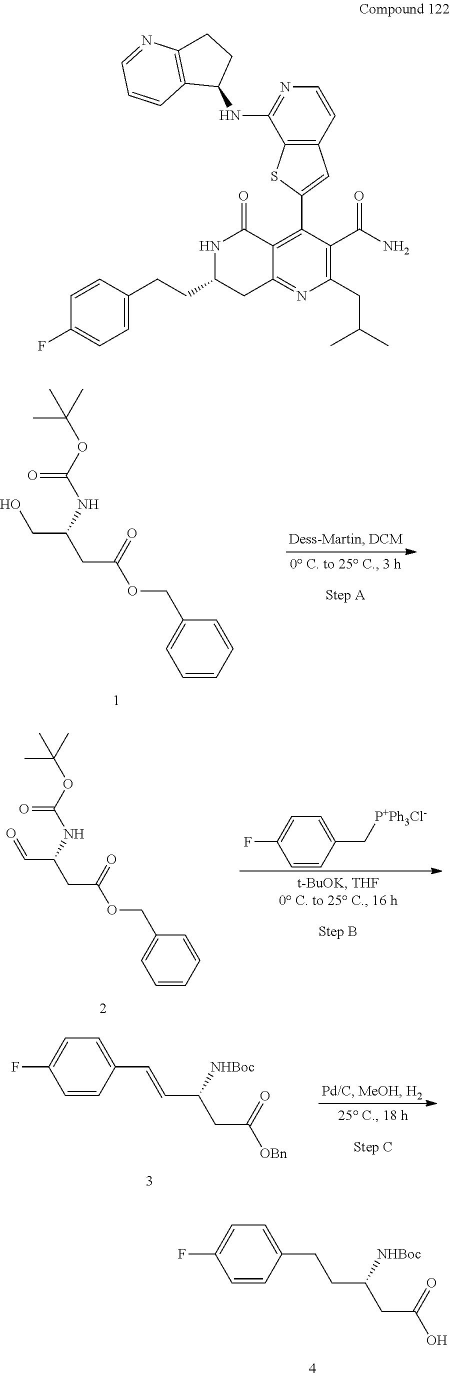

### Step A: benzyl(3R)-3-formyl-3-({[(2-methylprop-2-yl)oxy]carbonyl}amino)propanoate

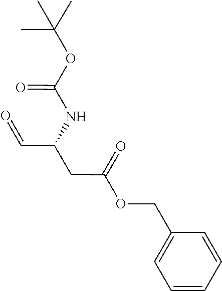 [SMILES: CC(C)(C)OC(=O)N[C@@H](C=O)CC(=O)OCc1ccccc1]

To a solution of benzyl(3R)-4-hydroxy-3-({[(2-methylprop-2-yl)oxy]carbonyl}amino)butanoate (1.7 g, 5.495 mmol) in DCM (20 mL) was added Dess-Martin periodinane (4.66 g, 10.990 mmol) at 0° C. The reaction mixture was stirred at 0° C. for 3 hours under N₂. The reaction mixture was washed with aq. NaHCO₃ (20 mL), the organic layer was dried over anhydrous Na₂SO₄, filtered and concentrated under vacuum. The resulting residue was purified by silica gel chromatography (eluting with PE/EA=2/1) to afford benzyl(3R)-3-formyl-3-({[(2-methylprop-2-yl)oxy]carbonyl}amino)propanoate (0.612 g, 36.24%).

### Step B: benzyl(3R,4E)-5-(4-fluorophenyl)-3-({[(2-methylprop-2-yl)oxy]carbonyl}amino)pent-4-enoate

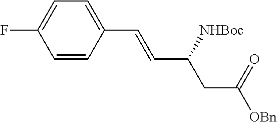 [SMILES: CC(C)(C)OC(=O)N[C@@H](/C=C/c1ccc(F)cc1)CC(=O)OCc1ccccc1]

To a solution of [(4-fluorophenyl)methyl]triphenylphosphanium chloride (810.17 mg, 1.991 mmol) in THF (20 mL) was added potassium 2-methylpropan-2-olate (335.15 mg, 2.987 mmol) under N₂. The reaction mixture was stirred at 0° C. for 5 min. Then benzyl(3R)-3-formyl-3-({[(2-methylprop-2-yl)oxy]carbonyl}amino)propanoate (612 mg, 1.991 mmol) in THF (5 mL) was added under N₂. The reaction mixture was stirred at 25° C. for another 16 hours. To the reaction mixture was added aq. NH₄Cl (50 mL). The resulting residue was extracted with EA (30 mL×3), the combined organic layers were washed with brine (20 mL), dried over anhydrous Na₂SO₄, filtered and concentrated under vacuum. The resulting residue was purified by silica gel chromatography (eluting with PE/EA=5/1) to afford benzyl(3R,4E)-5-(4-fluorophenyl)-3-({[(2-methylprop-2-yl)oxy]carbonyl}amino)pent-4-enoate (502 mg, 63.11%). LC-MS: m/z 422.4 (M+Na)⁺.

### Step C: (3S)-5-(4-fluorophenyl)-3-({[(2-methylprop-2-yl)oxy]carbonyl}amino)pentanoic acid

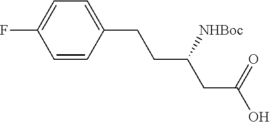 [SMILES: CC(C)(C)OC(=O)N[C@@H](CCc1ccc(F)cc1)CC(=O)O]

To a solution of benzyl(3R,4E)-5-(4-fluorophenyl)-3-({[(2-methylprop-2-yl)oxy]carbonyl}amino)pent-4-enoate (502 mg, 1.257 mmol) in MeOH (30 mL) was added Pd/C 10% (112.1 mg, 1.053 mmol). The reaction mixture was stirred at room temperature for 18 hours under H₂ balloon. The reaction mixture was filtered by Celite, the filtrate was concentrated under vacuum. The resulting residue was purified by C18 (eluting with 45% of ACN in water, 0.1% FA) to afford (3S)-5-(4-fluorophenyl)-3-({[(2-methylprop-2-yl)oxy]carbonyl}amino) pentanoic acid (230 mg, 58.78%). LC-MS: m/z 310.0 (M−H)⁻.

### (S)-4-{7-[(R)-4-aza-1-indanylamino]-1-thia-6-aza-2-indenyl}-7-[2-(p-fluorophenyl)ethyl]-2-isobutyl-5-oxo-5,6,7,8-tetrahydro-1,6-diaza-3-naphthamide (Compound 122)

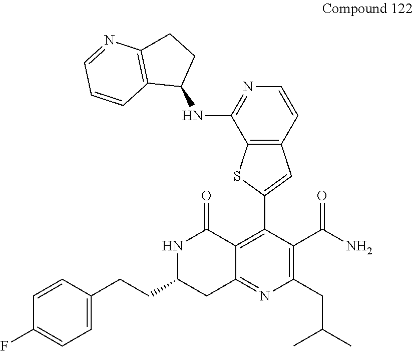 [SMILES: CC(C)Cc1nc2c(c(-c3cc4ccnc(N[C@@H]5CCc6ncccc65)c4s3)c1C(N)=O)C(=O)N[C@@H](CCc1ccc(F)cc1)C2]

Compound 122 was then synthesized following the similar route of Example 13, using (3S)-5-(4-fluorophenyl)-3-({[(2-methylprop-2-yl)oxy]carbonyl}amino) pentanoic acid in step F.

¹H NMR (400 MHz, DMSO-d₆) δ 8.50 (s, 1H), 8.18 (s, 1H), 7.91 (m, 3H), 7.83-7.52 (s, 1H), 7.24-7.37 (m, 5H), 7.10 (t, J=8.8 Hz, 2H), 5.73-5.75 (m, 1H), 3.18-3.23 (m, 2H), 2.97-3.03 (m, 2H), 2.66-2.68 (m, 5H), 2.31-2.33 (m, 1H), 2.24-2.28 (m, 1H), 2.15-2.19 (m, 1H), 1.78-1.86 (m, 3H), 0.92 (d, J=6.8 Hz, 6H). ¹⁹F NMR (376 MHz, DMSO-d₆) δ −117.42. LC-MS: m/z 635.1 (M+H)⁺.

### Example 15

### 4-{5-[N-(R)-4-fluoro-1-indanylcarbamoyl]-2-thienyl}-1-[(p-fluorophenyl)methyl]-6-isobutyl-1H-1,7-diazaindene-5-carboxamide (Compound 123)

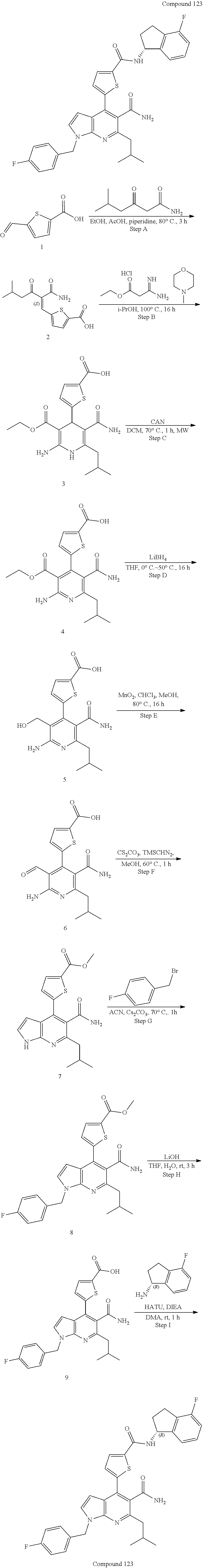

### Step A: (Z)-5-(2-carbamoyl-5-methyl-3-oxohex-1-en-1-yl)thiophene-2-carboxylic acid

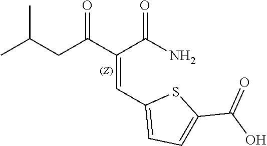 [SMILES: CC(C)CC(=O)/C(=C/c1ccc(C(=O)O)s1)C(N)=O]

To a solution of 5-formylthiophene-2-carboxylic acid (1 g, 6.41 mmol), 5-methyl-3-oxohexanamide (917 mg, 6.41 mmol) in EtOH (20 mL) were added piperidine (28 mg, 0.05 mmol) and AcOH (20 mg, 0.05 mmol), and the mixture was stirred at 80° C. for 3 hours. The reaction mixture was purified by reverse-phase flash chromatography (0.1 FA/H₂O:ACN=0 to 20%) to afford (Z)-5-(2-carbamoyl-5-methyl-3-oxohex-1-en-1-yl)thiophene-2-carboxylic acid (1.3 g, 72.2%). LC-MS: m/z 282.2 (M+H)⁺.

### Step B: ethyl (S)-4-((tert-butoxycarbonyl)amino)-5-(4-fluorophenyl)-3-oxopentanoate

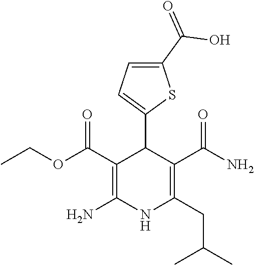 [SMILES: CCOC(=O)C1=C(N)NC(CC(C)C)=C(C(N)=O)C1c1ccc(C(=O)O)s1]

To a solution of (Z)-5-(2-carbamoyl-5-methyl-3-oxohex-1-en-1-yl)thiophene-2-carboxylic acid (1.3 g, 4.63 mmol) in i-PrOH (20 mL) were added ethyl 3-amino-3-iminopropanoate hydrochloride (773 mg, 4.63 mmol) and 4-methylmorpholine (470 mg, 4.63 mmol), the reaction mixture was stirred 5-(2-amino-5-carbamoyl-3-(ethoxycarbonyl)-6-isobutyl-1,4-dihydropyridin-4-yl)thiophene-2-carboxylic acid (1.7 g, 94.4%). LC-MS: m/z 394.0 (M+H)⁺.

### Step C: 5-(2-amino-5-carbamoyl-3-(ethoxycarbonyl)-6-isobutylpyridin-4-yl)thiophene-2-carboxylic acid

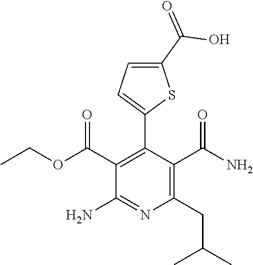 [SMILES: CCOC(=O)c1c(N)nc(CC(C)C)c(C(N)=O)c1-c1ccc(C(=O)O)s1]

To a solution of 5-(2-amino-5-carbamoyl-3-(ethoxycarbonyl)-6-isobutyl-1,4-dihydropyridin-4-yl)thiophene-2-carboxylic acid (1.7 g, 4.33 mmol) in DCM (17 mL) was added ceric ammonium nitrate (7.1 g, 12.98 mmol), and the reaction mixture was stirred at 70° C. for 1 hour in microwave. The reaction mixture was purified by reverse phase flash chromatography (0.1 FA/H₂O:ACN=0 to 10%) to afford 5-(2-amino-5-carbamoyl-3-(ethoxycarbonyl)-6-isobutylpyridin-4-yl)thiophene-2-carboxylic acid (1.6 g, 94.7%). LC-MS: m/z 391.9 (M+H)⁺.

### Step D: 5-(2-amino-5-carbamoyl-3-(hydroxymethyl)-6-isobutylpyridin-4-yl)thiophene-2-carboxylic acid

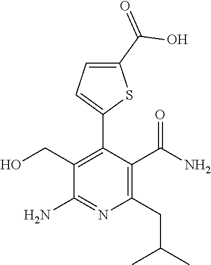 [SMILES: CC(C)Cc1nc(N)c(CO)c(-c2ccc(C(=O)O)s2)c1C(N)=O]

To a solution of 5-(2-amino-5-carbamoyl-3-(ethoxycarbonyl)-6-isobutylpyridin-4-yl)thiophene-2-carboxylic acid (370 mg, 0.90 mmol) in THF (15 mL) was added LiBH₄ (60 mg, 2.70 mmol), and the mixture was stirred at 50° C. for 16 hours under N₂. The reaction mixture was purified by reverse phase flash chromatography (0.1 FA/H₂O:ACN=0 to 10%) to afford 5-(2-amino-5-carbamoyl-3-(hydroxymethyl)-6-isobutylpyridin-4-yl)thiophene-2-carboxylic acid (300 mg, crude) which was used directly in the next step without further purification. LC-MS: m/z 350.1 (M+H)⁺.

### Step E: 5-(2-amino-5-carbamoyl-3-formyl-6-isobutylpyridin-4-yl)thiophene-2-carboxylic acid

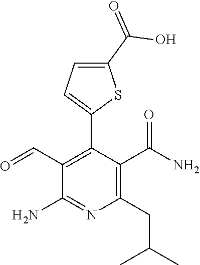 [SMILES: CC(C)Cc1nc(N)c(C=O)c(-c2ccc(C(=O)O)s2)c1C(N)=O]

To a solution of 5-(2-amino-5-carbamoyl-3-(hydroxymethyl)-6-isobutylpyridin-4-yl)thiophene-2-carboxylic acid (250 mg, 0.72 mmol) in CHCl₃/MeOH (30 mL/5 mL) was added MnO₂ (627 mg, 7.2 mmol), the mixture was stirred at 80° C. for 16 hours. After the reaction was completed, the organic layer was filtered and concentrated to afford 5-(2-amino-5-carbamoyl-3-formyl-6-isobutylpyridin-4-yl)thiophene-2-carboxylic acid (200 mg, 80.3%) which was used directly in the next step without further purification. LC-MS: m/z 347.9 (M+H)⁺.

### Step F: methyl 5-(5-carbamoyl-6-isobutyl-1H-pyrrolo[2,3-b]pyridin-4-yl)thiophene-2-carboxylate

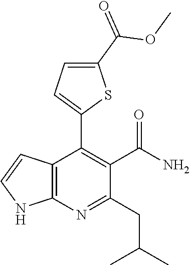 [SMILES: COC(=O)c1ccc(-c2c(C(N)=O)c(CC(C)C)nc3[nH]ccc23)s1]

A mixture of 5-(2-amino-5-carbamoyl-3-formyl-6-isobutylpyridin-4-yl)thiophene-2-carboxylic acid (80 mg, 0.23 mmol) and Cs₂CO₃ (150 mg, 0.46 mmol) in methanol (10 mL) was treated with TMSCHN₂ (2.3 mL, 4.6 mmol, 2.0 M) solution in hexanes (0.58 mL, 1.16 mmol) dropwise at 60° C. The mixture was stirred at 60° C. for 1 hour. The reaction mixture was concentrated and purified by Prep-TLC (EtOAc) to afford methyl 5-(5-carbamoyl-6-isobutyl-1H-pyrrolo[2,3-b]pyridin-4-yl)thiophene-2-carboxylate (35 mg, 42.7%). LC-MS: m/z 358.1 (M+H)⁺.

### Step G: methyl 5-(5-carbamoyl-1-(4-fluorobenzyl)-6-isobutyl-1H-pyrrolo[2,3-b]pyridin-4-yl)thiophene-2-carboxylate

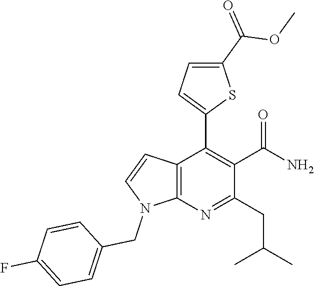 [SMILES: COC(=O)c1ccc(-c2c(C(N)=O)c(CC(C)C)nc3c2ccn3Cc2ccc(F)cc2)s1]

To a solution of methyl 5-(5-carbamoyl-6-isobutyl-1H-pyrrolo[2,3-b]pyridin-4-yl)thiophene-2-carboxylate (35 mg, 0.10 mmol) in ACN (5 mL) were added 1-(bromomethyl)-4-fluorobenzene (22 mg, 0.12 mmol), Cs₂CO₃ (98 mg, 0.30 mmol) and the reaction mixture was stirred at 70° C. for 1 hour. The reaction mixture was diluted with EA (50 mL), washed with brine (50 mL×2), concentrated, purified by Prep-TLC (EA:PE=1:1) to afford methyl 5-(5-carbamoyl-1-(4-fluorobenzyl)-6-isobutyl-1H-pyrrolo[2,3-b]pyridin-4-yl)thiophene-2-carboxylate (15 mg, 32.6%). LC-MS: m/z 358.1 (M+H)⁺.

### Step H: 5-(5-carbamoyl-1-(4-fluorobenzyl)-6-isobutyl-1H-pyrrolo[2,3-b]pyridin-4-yl)thiophene-2-carboxylic acid

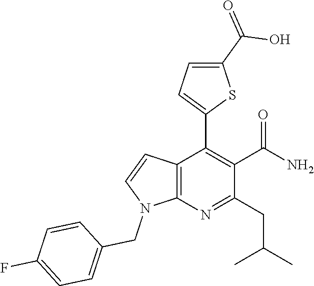 [SMILES: CC(C)Cc1nc2c(ccn2Cc2ccc(F)cc2)c(-c2ccc(C(=O)O)s2)c1C(N)=O]

To a solution of methyl 5-(5-carbamoyl-1-(4-fluorobenzyl)-6-isobutyl-1H-pyrrolo[2,3-b]pyridin-4-yl)thiophene-2-carboxylate (15 mg, 0.03 mmol) in THF/H₂O (6 mL/2 mL) was added LiOH (7 mg, 0.16 mmol), and the reaction mixture was stirred at room temperature for 3 hours. After the reaction was completed, the reaction mixture was diluted with H₂O (20 mL), adjusted with acetic acid (0.5 mL) until pH=5-6, extracted with EA (30 mL×2). The organic layer was washed with water (30 mL×2), dried over Na₂SO₄, filtered and concentrated to afford 5-(5-carbamoyl-1-(4-fluorobenzyl)-6-isobutyl-1H-pyrrolo[2,3-b]pyridin-4-yl)thiophene-2-carboxylic acid (12 mg, 82.8%) which was used directly in the next step without further purification. LC-MS: m/z 452.1 (M+H)⁺.

### Step I: 4-{5-[N-(R)-4-fluoro-1-indanylcarbamoyl]-2-thienyl}-1-[(p-fluorophenyl)methyl]-6-isobutyl-1H-1,7-diazaindene-5-carboxamide (Compound 123)

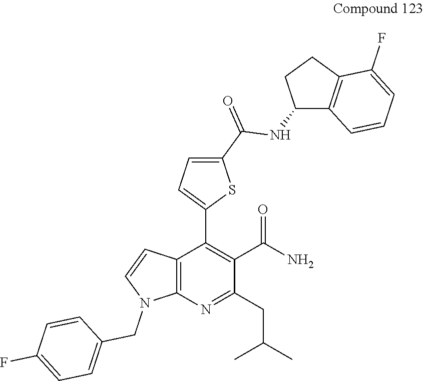 [SMILES: CC(C)Cc1nc2c(ccn2Cc2ccc(F)cc2)c(-c2ccc(C(=O)N[C@@H]3CCc4c(F)cccc43)s2)c1C(N)=O]

To a solution of 5-(5-carbamoyl-1-(4-fluorobenzyl)-6-isobutyl-1H-pyrrolo[2,3-b]pyridin-4-yl)thiophene-2-carboxylic acid (12 mg, 0.03 mmol) in DMA (1 mL) were added (R)-4-fluoro-2,3-dihydro-1H-inden-1-amine (6 mg, 0.032 mmol), HATU (12 mg, 0.032 mmol) and DIEA (12 mg, 0.032 mmol), the reaction mixture was stirred at room temperature for 1 hour. The reaction mixture was purified by Prep-HPLC with 0.1% FA to afford 4-{5-[N-(R)-4-fluoro-1-indanylcarbamoyl]-2-thienyl}-1-[(p-fluorophenyl)methyl]-6-isobutyl-1H-1,7-diazaindene-5-carboxamide (3.52 mg, 23.0%).

¹H NMR (400 MHz, CD₃OD) δ 7.77-7.78 (m, 1H), 7.49-7.50 (m, 1H), 7.41-7.42 (m, 1H), 7.42-7.34 (m, 3H), 7.14-7.16 (m, 1H), 7.00-7.04 (m, 3H), 6.66 (d, J=3.6 Hz, 1H), 5.68 (t, J=8.4 Hz, 1H), 5.52 (s, 2H), 3.13-3.20 (m, 1H), 2.91-2.97 (m, 1H), 2.88 (d, J=7.2 Hz, 2H), 2.61-2.73 (m, 1H), 2.39-2.46 (m, 1H), 2.05-2.15 (m, 1H), 0.78-0.99 (m, 6H). ¹⁹F NMR (376 MHz, CD₃OD) δ −117.26, −121.12. LC-MS: m/z 585.3 (M+H)⁺.

### 4-{5-[N-(R)-5-fluoro-1-indanylcarbamoyl]-2-thienyl}-1-[(p-fluorophenyl)methyl]-6-isobutyl-1H-1,7-diazaindene-5-carboxamide (Compound 124)

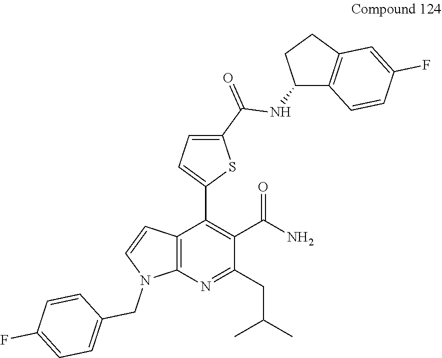 [SMILES: CC(C)Cc1nc2c(ccn2Cc2ccc(F)cc2)c(-c2ccc(C(=O)N[C@@H]3CCc4cc(F)ccc43)s2)c1C(N)=O]

Compound 124 was synthesized following the similar route of Example 15, using (R)-5-fluoro-2,3-dihydro-1H-inden-1-amine in step I.

¹H NMR (400 MHz, DMSO-d₆) δ 8.90 (d, J=8.0 Hz, 1H), 7.86 (d, J=3.6 Hz, 1H), 7.76 (s, 1H), 7.70 (d, J=3.6 Hz, 1H), 7.47-7.48 (m, 1H), 7.35-7.40 (m, 3H), 7.26-7.30 (m, 1H), 7.11-7.16 (m, 3H), 6.99-7.04 (m, 1H), 6.58 (d, J=3.6 Hz, 1H), 5.47-5.52 (m, 3H), 2.98-3.04 (m, 1H), 2.85-2.91 (m, 1H), 2.74-2.76 (m, 2H), 2.31-2.47 (m, 2H), 1.98-2.08 (m, 1H), 0.90-0.92 (m, 6H). ¹⁹F NMR (376 MHz, DMSO-d₆) δ −115.15, −115.82. LC-MS: m/z 585.2 (M+H)⁺.

### 4-{5-[N-(R)-6-fluoro-1-indanylcarbamoyl]-2-thienyl}-1-[(p-fluorophenyl)methyl]-6-isobutyl-1H-1,7-diazaindene-5-carboxamide (Compound 125)

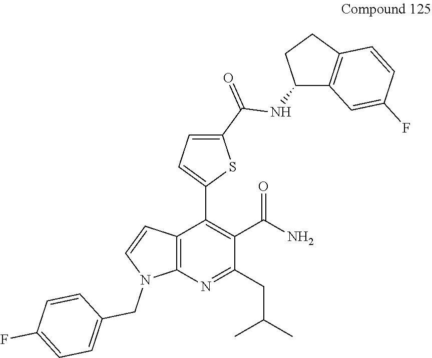 [SMILES: CC(C)Cc1nc2c(ccn2Cc2ccc(F)cc2)c(-c2ccc(C(=O)N[C@@H]3CCc4ccc(F)cc43)s2)c1C(N)=O]

Compound 125 was synthesized following the similar route of Example 15, using (R)-6-fluoro-2,3-dihydro-1H-inden-1-amine in step I.

¹H NMR (400 MHz, DMSO-d₆) δ 8.94 (d, J=8.4 Hz, 1H), 7.87 (d, J=4.0 Hz, 1H), 7.76-7.77 (m, 1H), 7.71 (d, J=3.6 Hz, 1H), 7.47-7.48 (m, 1H), 7.35-7.40 (m, 3H), 7.29-7.32 (m, 1H), 7.03-7.16 (m, 4H), 6.58 (d, J=3.6 Hz, 1H), 5.47-5.54 (m, 3H), 2.94-3.00 (m, 1H), 2.78-2.87 (m, 1H), 2.74-2.76 (m, 2H), 2.31-2.47 (m, 2H), 1.97-2.07 (m, 1H), 0.90-0.92 (m, 6H). ¹⁹F NMR (376 MHz, DMSO-d₆) δ −115.15, −115.95. LC-MS: m/z 585.2 (M+H)⁺.

### 4-{5-[N-(R)-1-indanylcarbamoyl]-2-thienyl}-1-[1-(p-fluorophenyl)ethyl]-6-isobutyl-1H-1,7-diazaindene-5-carboxamide (Compound 126)

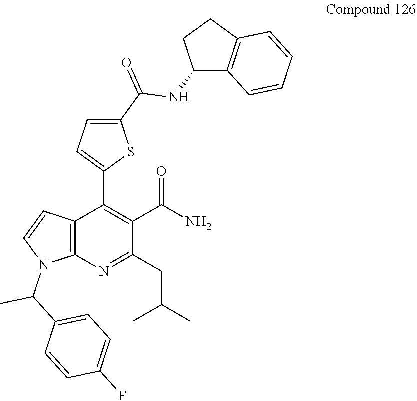 [SMILES: CC(C)Cc1nc2c(ccn2C(C)c2ccc(F)cc2)c(-c2ccc(C(=O)N[C@@H]3CCc4ccccc43)s2)c1C(N)=O]

Compound 126 was synthesized following the similar route of Example 15, using 1-(1-bromoethyl)-4-fluorobenzene in step G and (R)-2,3-dihydro-1H-inden-1-amine in step I.

¹H NMR (400 MHz, DMSO-d₆) δ 8.90 (d, J=8.4 Hz, 1H), 7.86-7.87 (m, 1H), 7.82-7.83 (m, 1H), 7.73-7.74 (m, 1H), 7.45-7.46 (m, 1H), 7.37-7.40 (m, 3H) 7.19-7.30 (m, 4H), 7.11-7.15 (m, 2H), 6.60 (d, J=3.6 Hz, 1H), 6.13-6.19 (m, 1H), 5.51-5.57 (m, 1H), 2.97-3.04 (m, 1H), 2.83-2.91 (m, 1H), 2.72-2.74 (m, 2H), 2.28-2.48 (m, 2H), 1.91-2.03 (m, 4H), 0.88-0.92 (m, 6H). ¹⁹F NMR (376 MHz, DMSO-d₆) δ −115.37. LC-MS: m/z 581.2 (M+H)⁺.

### 4-{5-[N-(R)-1-indanylcarbamoyl]-2-thienyl}-1-[(1,3-benzothiazol-6-yl)methyl]-6-isobutyl-1H-1,7-diazaindene-5-carboxamide (Compound 127)

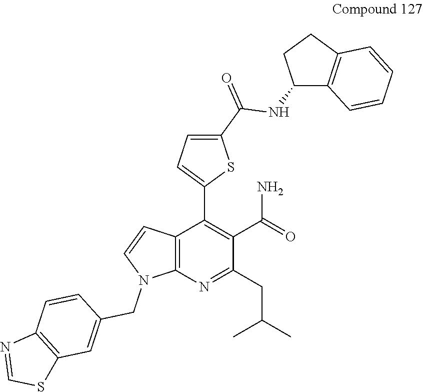 [SMILES: CC(C)Cc1nc2c(ccn2Cc2ccc3ncsc3c2)c(-c2ccc(C(=O)N[C@@H]3CCc4ccccc43)s2)c1C(N)=O]

Compound 127 was synthesized following the similar route of Example 15, using 6-(bromomethyl)benzo[d]thiazole in step G and (R)-2,3-dihydro-1H-inden-1-amine in step I.

¹H NMR (400 MHz, DMSO-d₆) δ 9.36 (s, 1H), 8.91 (d, J=8.4 Hz, 1H), 8.15 (s, 1H), 8.01-8.03 (m, 1H), 7.86-7.87 (m, 1H), 7.75-7.77 (m, 2H), 7.47-7.52 (m, 2H), 7.39 (d, J=3.6 Hz, 1H), 7.19-7.30 (m, 4H), 6.60 (d, J=3.6 Hz, 1H), 5.46 (s, 2H), 5.51-557 (m, 1H), 2.97-3.04 (m, 1H), 2.83-2.91 (m, 1H), 2.75-2.77 (m, 2H), 2.32-2.48 (m, 2H), 1.93-2.02 (m, 1H), 0.89-0.91 (m, 6H). LC-MS: m/z 606.2 (M+H)⁺.

### Example 16

### 4-{5-[N-(R)-1-indanylcarbamoyl]-2-thienyl}-1-[(5-indanyl)methyl]-6-isobutyl-1H-1,7-diazaindene-5-carboxamide (Compound 128)

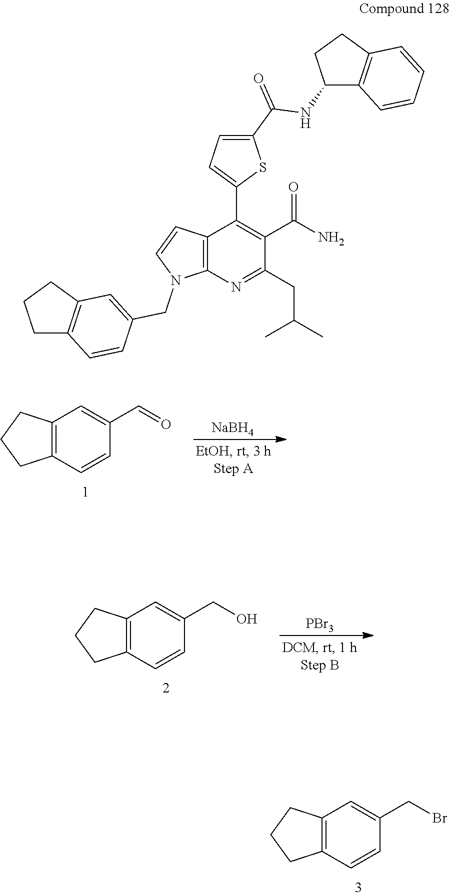

### Step A: (2,3-dihydro-1H-inden-5-yl)methanol

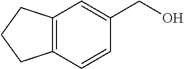 [SMILES: OCc1ccc2c(c1)CCC2]

To a solution of 2,3-dihydro-1H-indene-5-carbaldehyde (230 mg, 1.58 mmol) in EtOH (5 mL), was added NaBH₄ (238 mg, 6.29 mmol) at 0° C., the mixture was stirred at room temperature for 3 hours. After the reaction was completed, the mixture was purified by Prep-TLC (PE:EA=5:1) to afford (2,3-dihydro-1H-inden-5-yl) methanol (190 mg, 81.48%).

¹H NMR (400 MHz, DMSO-d₆) δ 7.14-7.16 (m, 2H), 7.03-7.05 (m, 1H), 5.03-5.06 (m, 1H), 4.44 (d, J=5.6 Hz, 2H), 2.80-2.84 (m, 4H), 1.96-2.03 (m, 2H)

### Step B: 5-(bromomethyl)-2,3-dihydro-1H-indene

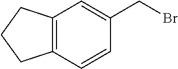 [SMILES: BrCc1ccc2c(c1)CCC2]

To a solution of (2,3-dihydro-1H-inden-5-yl) methanol (190 mg, 1.28 mmol) in DCM (5 mL), was added PBr₃ (139 mg, 0.51 mmol) at 0° C., the mixture was stirred at room temperature for 1 hour. After the reaction was completed. The mixture was purified by prep-TLC (PE:EA=10:1) to afford 5-(bromomethyl)-2,3-dihydro-1H-indene (210 mg, 77.6%). ¹H NMR (400 MHz, DMSO-d₆) δ 7.28 (s, 1H), 7.18-7.20 (m, 2H), 4.67 (s, 2H), 2.81-2.85 (m, 4H), 2.01 (dd, J=14.8 Hz, 7.2 Hz, 2H).

### 4-{5-[N-(R)-1-indanylcarbamoyl]-2-thienyl}-1-[(5-indanyl)methyl]-6-isobutyl-1H-1,7-diazaindene-5-carboxamide (Compound 128)

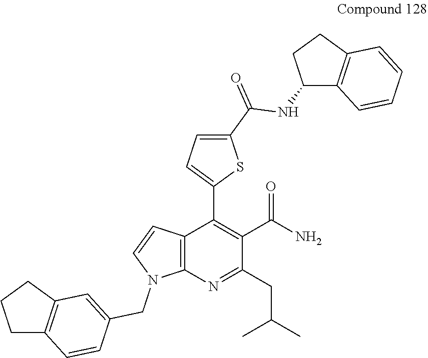 [SMILES: CC(C)Cc1nc2c(ccn2Cc2ccc3c(c2)CCC3)c(-c2ccc(C(=O)N[C@@H]3CCc4ccccc43)s2)c1C(N)=O]

Compound 128 was then synthesized following the similar route of Example 15, using 5-(bromomethyl)-2,3-dihydro-1H-indene in step G and (R)-2,3-dihydro-1H-inden-1-amine in step I.

¹H NMR (400 MHz, CD₃OD) δ 7.77 (d, J=3.6 Hz, 1H), 7.45 (d, J=3.6 Hz, 1H), 7.42 (d, J=4.0 Hz, 1H), 7.31-7.33 (m, 1H), 7.21-7.30 (m, 3H), 7.11-7.13 (m, 2H), 7.05-7.07 (m, 1H), 6.63 (d, J=3.6 Hz, 1H), 5.63-5.67 (m, 1H), 5.49 (s, 2H), 3.07-3.14 (m, 1H), 2.08-2.90 (m, 7H), 2.57-2.65 (m, 1H), 2.40-2.47 (m, 1H), 2.00-2.10 (m, 3H), 1.00 (d, J=6.4 Hz, 6H). LC-MS: m/z 589.3 (M+H)⁺.

### Example 17

### 4-(5-{[(3,4-difluorophenyl)methyl]carbamoyl}-2-thienyl)-1-[(p-fluorophenyl)methyl]-6-isobutyl-3-vinyl-1H-1,7-diazaindene-5-carboxamide (Compound 129) and 4-(5-{[(3,4-difluorophenyl)methyl]carbamoyl}-2-thienyl)-3-ethyl-1-[(p-fluorophenyl)methyl]-6-isobutyl-1H-1,7-diazaindene-5-carboxamide (Compound 130)

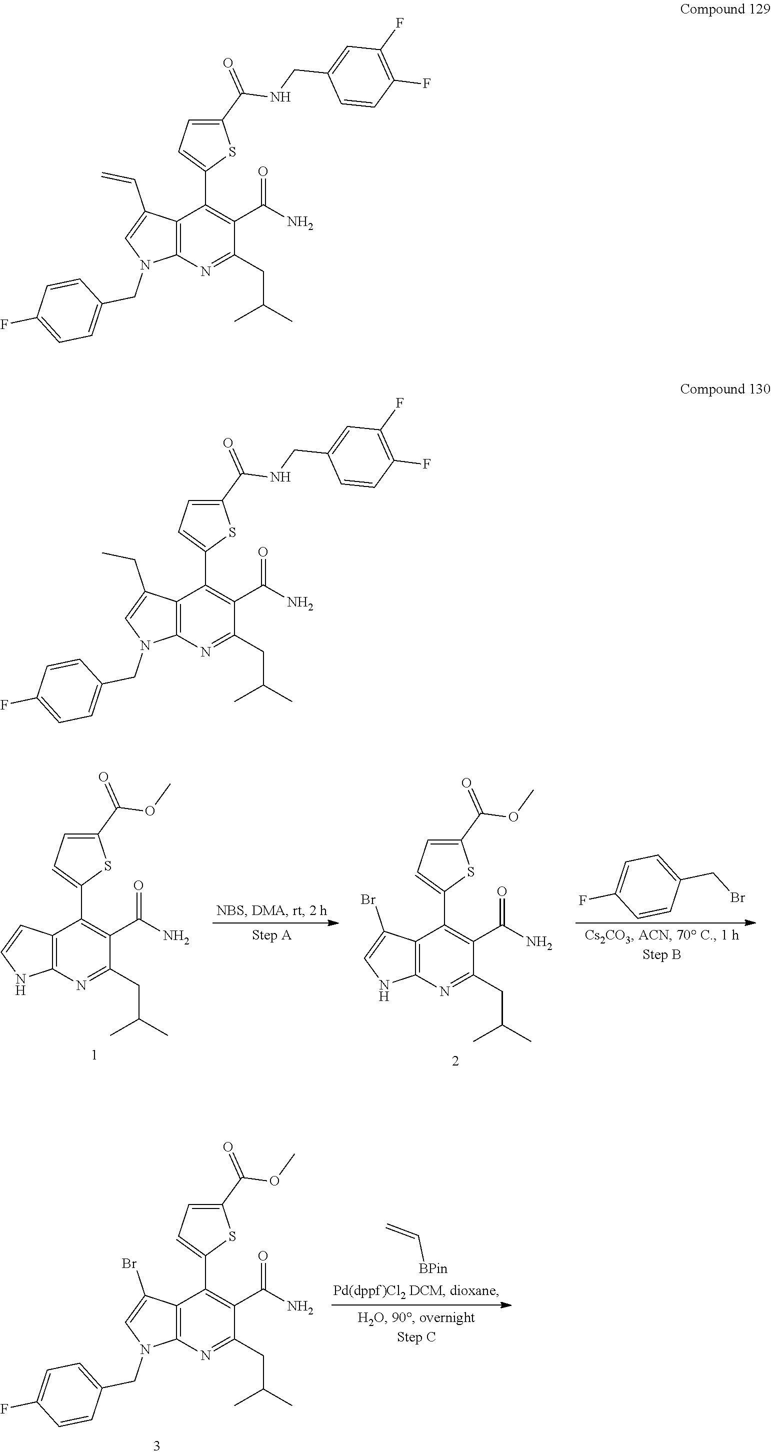

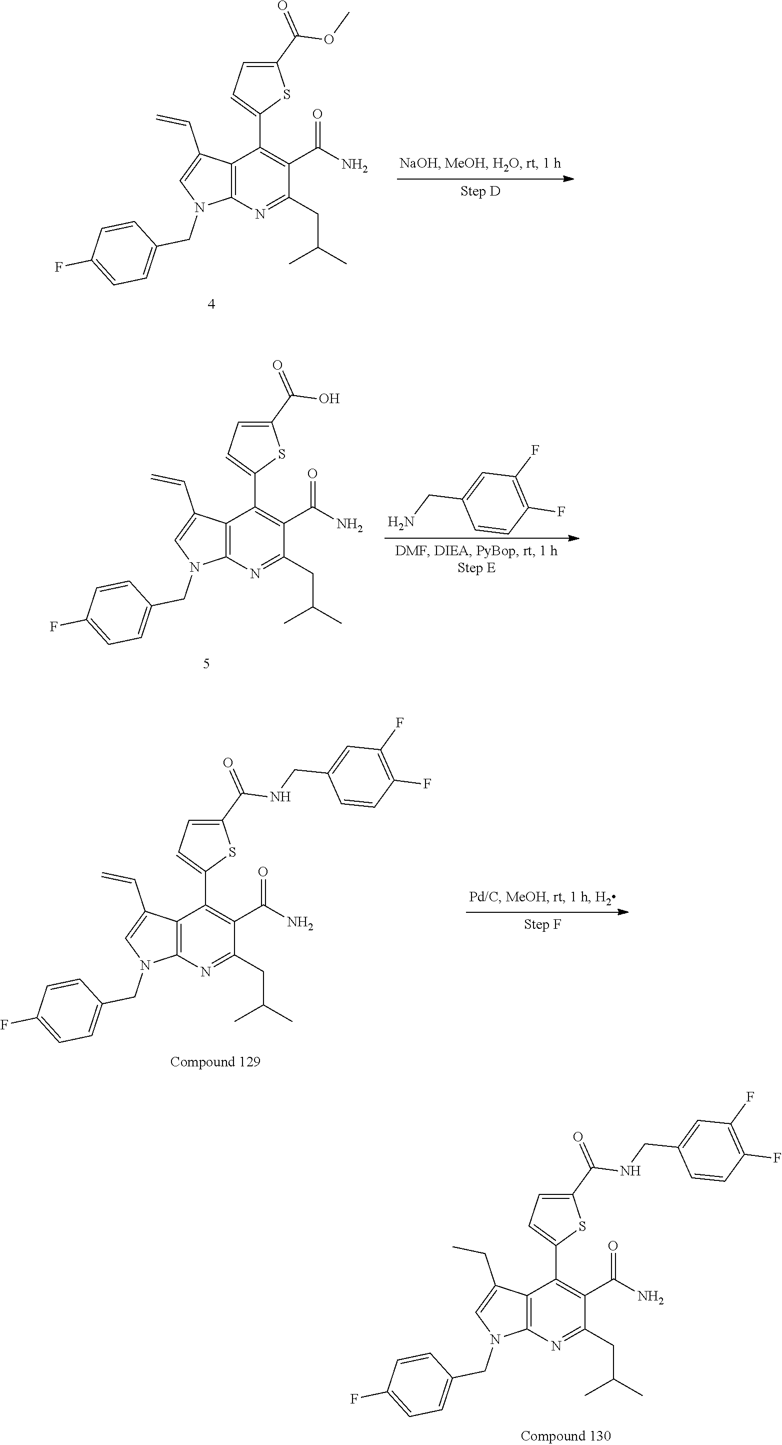 [SMILES: C=Cc1cn(Cc2ccc(F)cc2)c2nc(CC(C)C)c(C(N)=O)c(-c3ccc(C(=O)OC)s3)c12 ; C=Cc1cn(Cc2ccc(F)cc2)c2nc(CC(C)C)c(C(N)=O)c(-c3ccc(C(=O)O)s3)c12 ; NCc1ccc(F)c(F)c1 ; C=Cc1cn(Cc2ccc(F)cc2)c2nc(CC(C)C)c(C(N)=O)c(-c3ccc(C(=O)NCc4ccc(F)c(F)c4)s3)c12 ; CCc1cn(Cc2ccc(F)cc2)c2nc(CC(C)C)c(C(N)=O)c(-c3ccc(C(=O)NCc4ccc(F)c(F)c4)s3)c12]

### Step A: methyl 5-(3-bromo-5-carbamoyl-6-isobutyl-1H-pyrrolo[2,3-b]pyridin-4-yl)thiophene-2-carboxylate

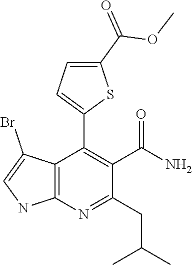 [SMILES: COC(=O)c1ccc(-c2c(C(N)=O)c(CC(C)C)nc3[nH]cc(Br)c23)s1]

To a solution of methyl 5-(5-carbamoyl-6-isobutyl-1H-pyrrolo[2,3-b]pyridin-4-yl)thiophene-2-carboxylate (210 mg, 0.588 mmol) in DMA (5 mL) was added NBS (125.49 mg, 0.705 mmol), and the reaction mixture was stirred at room temperature for 1.5 hour. After the reaction was completed, the mixture was diluted with EA (30 mL), washed with water (60 mL). The organic layers were dried over Na₂SO₄, filtered and concentrated in vacuum to dryness. The residue was purified by column chromatography on silica gel (EA/PE=35/65) to afford methyl 5-(3-bromo-5-carbamoyl-6-isobutyl-1H-pyrrolo[2,3-b]pyridin-4-yl)thiophene-2-carboxylate (151 mg, 58.90%). LC-MS: m/z 436.0 (M+H)⁺.

### Step B: methyl 5-(3-bromo-5-carbamoyl-1-(4-fluorobenzyl)-6-isobutyl-1H-pyrrolo[2,3-b]pyridin-4-yl)thiophene-2-carboxylate

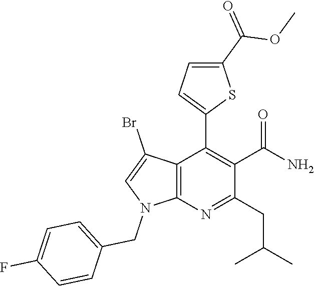 [SMILES: COC(=O)c1ccc(-c2c(C(N)=O)c(CC(C)C)nc3c2c(Br)cn3Cc2ccc(F)cc2)s1]

To a solution of methyl 5-(3-bromo-5-carbamoyl-6-isobutyl-1H-pyrrolo[2,3-b]pyridin-4-yl)thiophene-2-carboxylate (130 mg, 0.298 mmol) in ACN (3 mL) were added Cs₂CO₃ (291.23 mg, 0.894 mmol) and 1-(bromomethyl)-4-fluorobenzene (67.58 mg, 0.358 mmol), and the reaction mixture was stirred at 70° C. for 1 hour. After the reaction was completed, the mixture was filtered and concentrated under vacuum to dryness. The residue was purified by column chromatography on silica gel (PE/EA=3/1) to afford methyl 5-(3-bromo-5-carbamoyl-1-(4-fluorobenzyl)-6-isobutyl-1H-pyrrolo[2,3-b]pyridin-4-yl)thiophene-2-carboxylate (155 mg, 95.56%). LC-MS: m/z 544.1 (M+H)⁺.

### Step C: methyl 5-(5-carbamoyl-1-(4-fluorobenzyl)-6-isobutyl-3-vinyl-1H-pyrrolo[2,3-b]pyridin-4-yl)thiophene-2-carboxylate

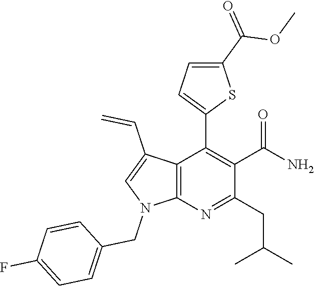 [SMILES: C=Cc1cn(Cc2ccc(F)cc2)c2nc(CC(C)C)c(C(N)=O)c(-c3ccc(C(=O)OC)s3)c12]

To a solution of methyl 5-(3-bromo-5-carbamoyl-1-(4-fluorobenzyl)-6-isobutyl-1H-pyrrolo[2,3-b]pyridin-4-yl)thiophene-2-carboxylate (50 mg, 0.092 mmol) in dioxane (2 mL) and H₂O (0.2 mL) were added 4,4,5,5-tetramethyl-2-vinyl-1,3,2-dioxaborolane (16.97 mg, 0.110 mmol), K₂CO₃ (38.08 mg, 0.276 mmol), and Pd(dppf)Cl₂·CH₂Cl₂ (7.50 mg, 0.009 mmol), and the reaction mixture was stirred at 90° C. overnight under N₂. After the reaction was completed, the mixture was filtered and concentrated under vacuum to dryness. The residue was purified by Prep-TLC (PE/EA=1/1) to afford methyl 5-(5-carbamoyl-1-(4-fluorobenzyl)-6-isobutyl-3-vinyl-1H-pyrrolo[2,3-b]pyridin-4-yl)thiophene-2-carboxylate (40 mg, 88.59%). LC-MS: m/z 492.1 (M+H)⁺.

### Step D: 5-(5-carbamoyl-1-(4-fluorobenzyl)-6-isobutyl-3-vinyl-1H-pyrrolo[2,3-b]pyridin-4-yl)thiophene-2-carboxylic acid

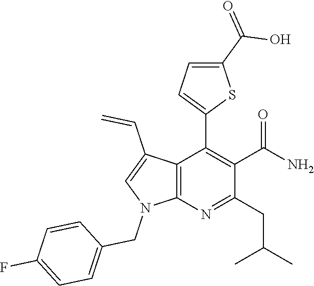 [SMILES: C=Cc1cn(Cc2ccc(F)cc2)c2nc(CC(C)C)c(C(N)=O)c(-c3ccc(C(=O)O)s3)c12]

A mixture of methyl 5-(5-carbamoyl-1-(4-fluorobenzyl)-6-isobutyl-3-vinyl-1H-pyrrolo[2,3-b]pyridin-4-yl)thiophene-2-carboxylate (35 mg, 0.071 mmol) and NaOH (85.44 mg, 2.136 mmol) in MeOH (1 mL) and H₂O (0.1 mL) was stirred at room temperature for 1 hour. After the reaction was completed, the mixture was diluted with EA (10 mL), adjusted pH to 6 with HCl (1 M), washed with H₂O (15 mL). The organic layers were dried over Na₂SO₄, filtered and concentrated to afford 5-(5-carbamoyl-1-(4-fluorobenzyl)-6-isobutyl-3-vinyl-1H-pyrrolo[2,3-b]pyridin-4-yl)thiophene-2-carboxylic acid (38 mg, crude) which was used directly in the next step without further purification. LC-MS: m/z 492.1 (M+H)⁺.

### Step E: 4-(5-{[(3,4-difluorophenyl)methyl]carbamoyl}-2-thienyl)-1-[(p-fluorophenyl)methyl]-6-isobutyl-3-vinyl-1H-1,7-diazaindene-5-carboxamide (Compound 129)

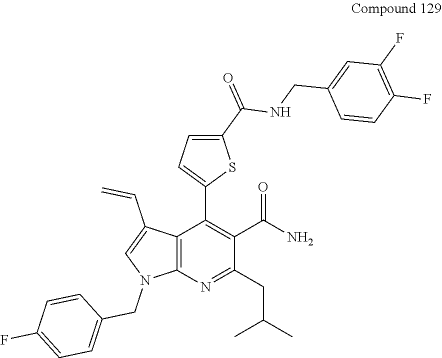 [SMILES: C=Cc1cn(Cc2ccc(F)cc2)c2nc(CC(C)C)c(C(N)=O)c(-c3ccc(C(=O)NCc4ccc(F)c(F)c4)s3)c12]

To a solution of 5-(5-carbamoyl-1-(4-fluorobenzyl)-6-isobutyl-3-vinyl-1H-pyrrolo[2,3-b]pyridin-4-yl)thiophene-2-carboxylic acid (30 mg, 0.063 mmol) in DMF (1 mL) were added DIEA (24.36 mg, 0.188 mmol), (3,4-difluorophenyl) methanamine (10.79 mg, 0.075 mmol), and PyBOP (39.23 mg, 0.075 mmol), and the reaction mixture was stirred at room temperature for 1 hour. After the reaction was completed, the mixture was diluted with water EA (20 mL), washed with water (40 mL). The organic layers were dried over Na₂SO₄, filtered and concentrated under vacuum to dryness. The residue was purified by Prep-HPLC to afford 4-(5-{[(3,4-difluorophenyl)methyl]carbamoyl}-2-thienyl)-1-[(p-fluorophenyl)methyl]-6-isobutyl-3-vinyl-1H-1,7-diazaindene-5-carboxamide (2.05 mg, 20.5%).

¹H NMR (400 MHz, DMSO-d₆) δ 9.17 (t, J=6.0 Hz, 1H), 8.00 (br s, 1H), 7.79 (d, J=4.0 Hz, 1H), 7.65 (br s, 1H), 7.31-7.47 (m, 5H), 7.09-7.24 (m, 4H), 5.89 (dd, J=11.2 Hz, J=17.6 Hz, 1H), 5.46 (s, 2H), 5.34 (d, J=17.6 Hz, 1H), 4.79 (d, J=12.4 Hz, 1H), 4.45 (d, J=6.0 Hz, 2H), 2.73 (d, J=7.2 Hz, 2H), 2.28-2.38 (m, 1H), 0.91 (d, J=6.4 Hz, 6H). ¹⁹F NMR (376 MHz, DMSO-d₆) δ −114.96, −138.87, −141.38. LC-MS: m/z 603.2 (M+H)⁺.

### Step F: 4-(5-{[(3,4-difluorophenyl)methyl]carbamoyl}-2-thienyl)-3-ethyl-1-[(p-fluorophenyl)methyl]-6-isobutyl-1H-1,7-diazaindene-5-carboxamide (Compound 130)

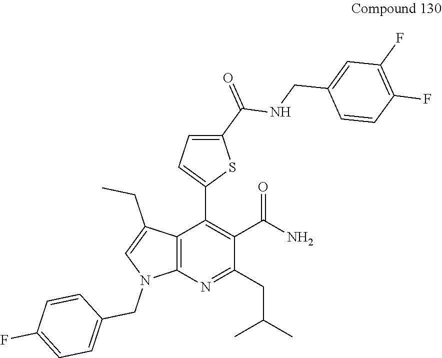 [SMILES: CCc1cn(Cc2ccc(F)cc2)c2nc(CC(C)C)c(C(N)=O)c(-c3ccc(C(=O)NCc4ccc(F)c(F)c4)s3)c12]

To a solution of 4-(5-{[(3,4-difluorophenyl)methyl]carbamoyl}-2-thienyl)-1-[(p-fluorophenyl)methyl]-6-isobutyl-3-vinyl-1H-1,7-diazaindene-5-carboxamide (17 mg, 0.028 mmol) in MeOH (1 mL) was added Pd/C (9.01 mg, 0.085 mmol, 10% Pd, wetted with ca. 55% water) and the reaction mixture was stirred at room temperature for 1 hour under H₂. After the reaction was completed, the mixture was filtered and concentrated under vacuum to dryness. The residue was purified by Prep-HPLC to afford 4-(5-{[(3,4-difluorophenyl)methyl]carbamoyl}-2-thienyl)-3-ethyl-1-[(p-fluorophenyl)methyl]-6-isobutyl-1H-1,7-diazaindene-5-carboxamide (2.17 mg, 12.72%).

¹H NMR (400 MHz, DMSO-d₆) δ 9.17 (t, J=6.0 Hz, 1H), 8.00 (br s, 1H), 7.79 (d, J=4.0 Hz, 1H), 7.65 (br s, 1H), 7.31-7.47 (m, 5H), 7.09-7.24 (m, 4H), 5.89 (dd, J=11.2 Hz, J=17.6 Hz, 1H), 5.46 (s, 2H), 5.34 (d, J=17.6 Hz, 1H), 4.79 (d, J=12.4 Hz, 1H), 4.45 (d, J=6.0 Hz, 2H), 2.73 (d, J=7.2 Hz, 2H), 2.28-2.38 (m, 1H), 0.91 (d, J=6.4 Hz, 6H). ¹⁹F NMR (376 MHz, DMSO-d₆) δ −115.23, −138.87, −141.38. LC-MS: m/z 605.1 (M+H)⁺.

### Example 18

### 4-{5-[N-(R)-1-indanylcarbamoyl]-2-thienyl}-1-[(p-fluorophenyl)methyl]-6-isobutyl-3-methyl-1H-1,2,7-triazaindene-5-carboxamide (Compound 131)

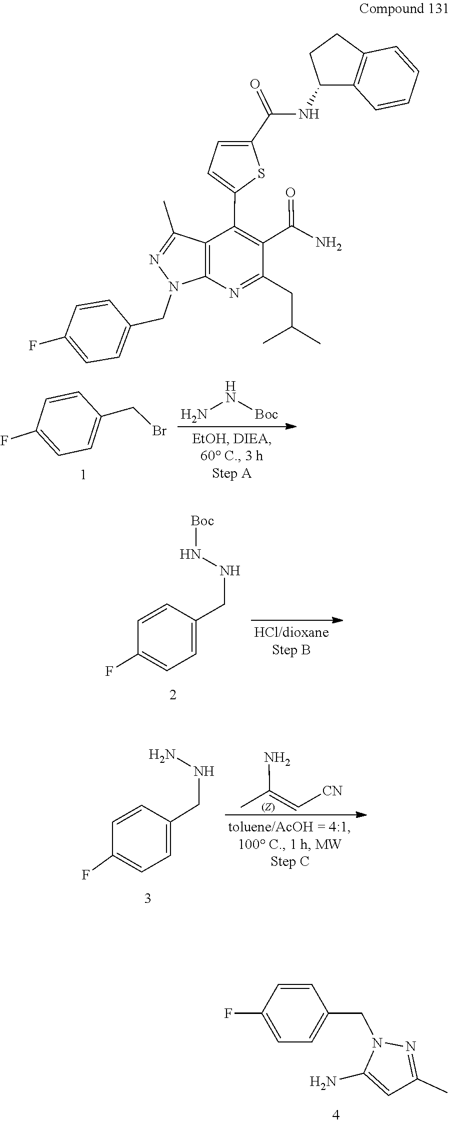

### Step A: tert-butyl 2-(4-fluorobenzyl)hydrazine-1-carboxylate

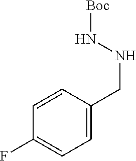 [SMILES: CC(C)(C)OC(=O)NNCc1ccc(F)cc1]

To a solution of 1-(bromomethyl)-4-fluorobenzene (1 g, 5.32 mmol) in EtOH (15 mL) were added tert-butyl hydrazinecarboxylate (5.10 g, 21.28 mmol) and DIEA (2.04 g, 15.96 mmol), the reaction mixture was stirred at 60° C. for 3 hours. The mixture was concentrated and purified by silica gel column chromatography eluting with (PF:FA=0~50%) to afford tert-butyl 2-(4-fluorobenzyl)hydrazine-1-carboxylate (800 mg, 62.5%). LC-MS: m/z 185.1 (M+H-^{t}Bu)⁺.

### Step B: (4-fluorobenzyl)hydrazine

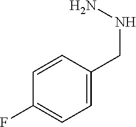

A solution of tert-butyl 2-(4-fluorobenzyl)hydrazine-1-carboxylate (800 g, 3.33 mmol) in HCl/dioxane (20 mL) was stirred at room temperature for 1 hour. After the reaction was completed, the mixture was concentrated to afford (4-fluorobenzyl)hydrazine (400 mg, 85.8%) which was used directly in the next step without further purification. LC-MS: m/z 141.2 (M−H)⁺.

### Step C: 1-(4-fluorobenzyl)-3-methyl-1H-pyrazol-5-amine

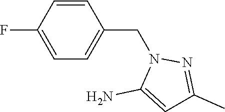

To a solution of (4-fluorobenzyl)hydrazine (400 mg, 2.16 mmol) in toluene/AcOH (16 mL/4 mL) was added (Z)-3-aminobut-2-enenitrile (89 mg, 1.08 mmol), the reaction mixture was stirred at 100° C. with N₂ for 30 min in microwave. The reaction mixture was filtered and concentrated in vacuo, purified by reversed phase flash chromatography (0.1% FA) to afford 1-(4-fluorobenzyl)-3-methyl-1H-pyrazol-5-amine (131 mg, 22.4%). LC-MS: m/z 206.1 (M+H).

### 4-{5-[N-(R)-1-indanylcarbamoyl]-2-thienyl}-1-[(p-fluorophenyl)methyl]-6-isobutyl-3-methyl-1H-1,2,7-triazaindene-5-carboxamide (Compound 131)

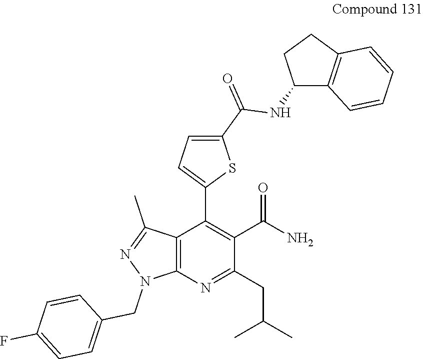 [SMILES: Cc1nn(Cc2ccc(F)cc2)c2nc(CC(C)C)c(C(N)=O)c(-c3ccc(C(=O)N[C@@H]4CCc5ccccc54)s3)c12]

Compound 131 was then synthesized following the similar route of Example 1, using 1-(4-fluorobenzyl)-3-methyl-1H-pyrazol-5-amine and (R)-N-(2,3-dihydro-1H-inden-1-yl)-5-formylthiophene-2-carboxamide in step D. ¹H NMR (400 MHz, DMSO-d₆) δ 8.94 (d, J=8.4 Hz, 1H), 7.86-7.87 (m, 1H), 7.77-7.78 (m, 1H), 7.49-7.50 (m, 1H), 7.32-7.35 (m, 2H), 7.27-7.32 (m, 2H), 7.17-7.30 (m, 3H), 7.12-7.15 (m, 2H), 5.59 (s, 2H), 5.50-5.56 (m, 1H), 2.99-3.05 (m, 1H), 2.83-2.91 (m, 1H), 2.78-2.80 (m, 2H), 2.33-2.47 (m, 2H), 2.06 (s, 3H), 1.93-2.01 (m, 1H), 0.94 (d, J=6.8 Hz, 6H), ¹⁹F NMR (376 MHz, DMSO-d₆) δ −114.93. LC-MS: m/z 582.2 (M+H)⁺.

### Example 19

### 7-(5-{[(3,4-difluorophenyl)methyl]carbamoyl}-2-thienyl)-3-[2-(p-fluorophenyl)ethyl]-5-isobutyl-3H-1,3,4-triazaindene-6-carboxamide (Compound 132)

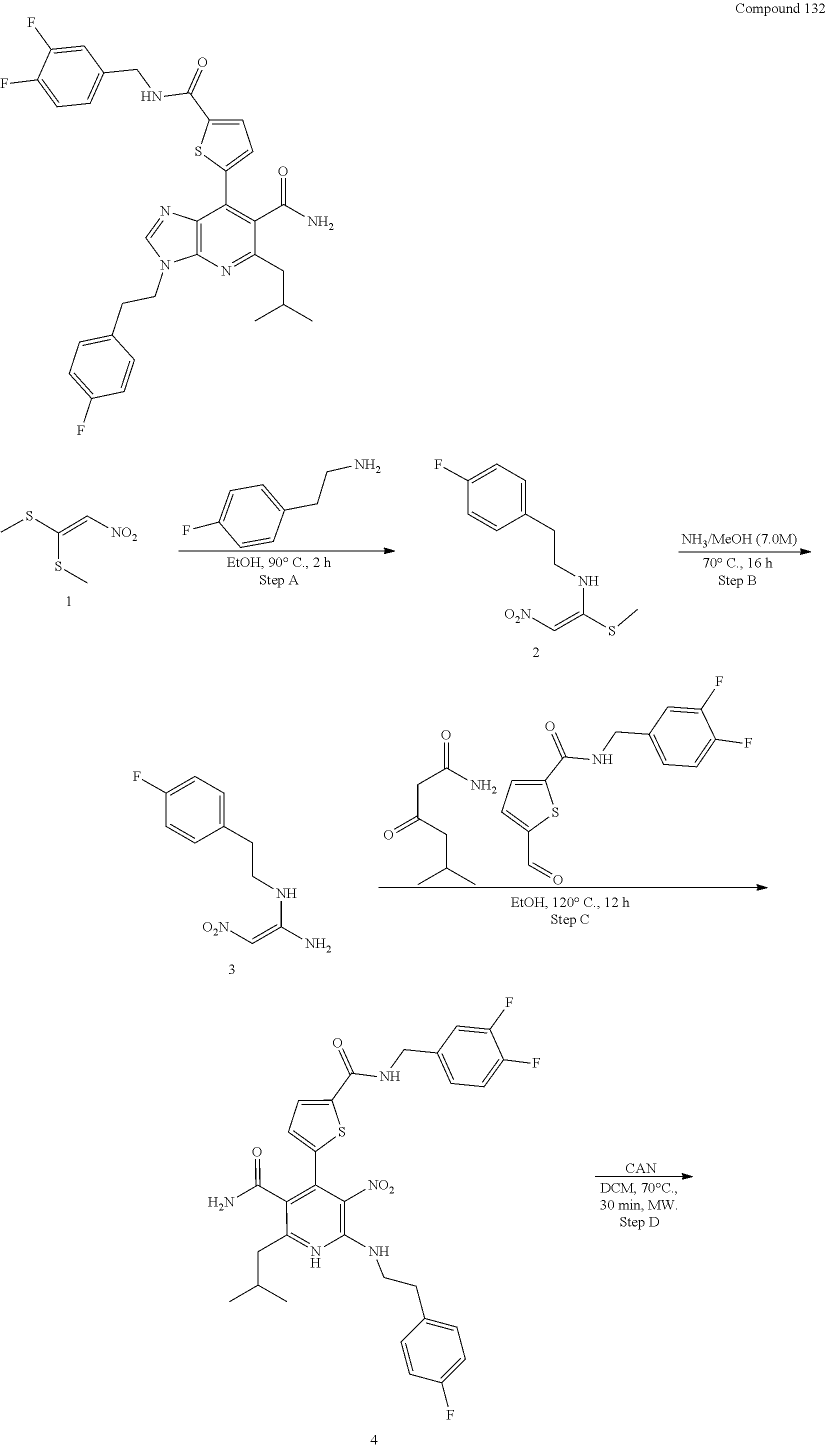

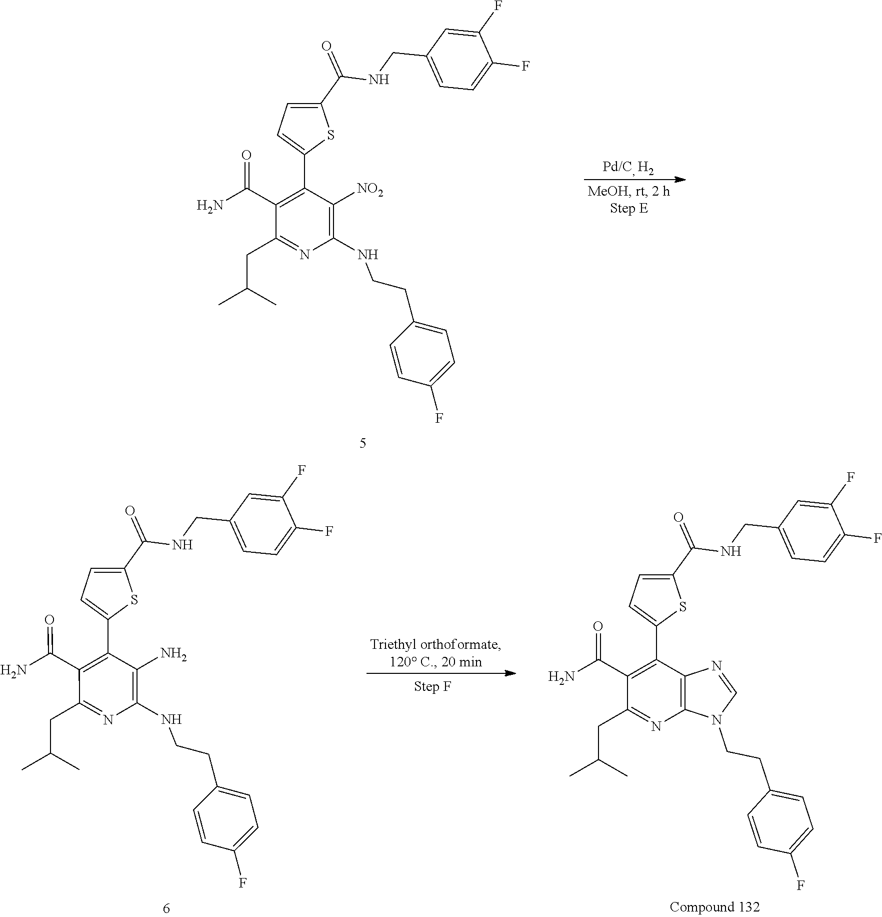 [SMILES: CC(C)Cc1nc(NCCc2ccc(F)cc2)c([N+](=O)[O-])c(-c2ccc(C(=O)NCc3ccc(F)c(F)c3)s2)c1C(N)=O ; "Pd/C, H2" ; CC(C)Cc1nc(NCCc2ccc(F)cc2)c(N)c(-c2ccc(C(=O)NCc3ccc(F)c(F)c3)s2)c1C(N)=O ; CC(C)Cc1nc2c(ncn2CCc2ccc(F)cc2)c(-c2ccc(C(=O)NCc3ccc(F)c(F)c3)s2)c1C(N)=O]

### Step A: (E)-N-(4-fluorophenethyl)-1-(methylthio)-2-nitroethen-1-amine

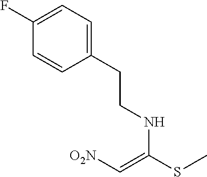 [SMILES: CS/C(=C/[N+](=O)[O-])NCCc1ccc(F)cc1]

A mixture of 3-(nitromethylidene)-2,4-dithiapentane (1 g, 6.05 mmol) in EtOH (7 mL) were added 2-(4-fluorophenyl) ethan-1-amine (925 mg, 0.67 mmol) and the reaction was stirred at 90° C. for 2 hours. The reaction was concentrated in vacuum to afford (E)-N-(4-fluorophenethyl)-1-(methylthio)-2-nitroethen-1-amine (1.2 g, crude) which was used directly in the next step without further purification.

### Step B: (Z)-N-(4-fluorophenethyl)-2-nitroethene-1,1-diamine

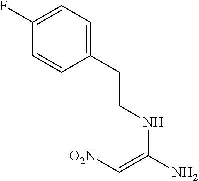 [SMILES: N/C(=C/[N+](=O)[O-])NCCc1ccc(F)cc1]

A mixture of (E)-N-(4-fluorophenethyl)-1-(methylthio)-2-nitroethen-1-amine (1.2 g, 4.68 mmol) in NH₃/MeOH (5 mL, 7 M) was stirred at 70° C. for 16 hours in a scaled tube. After the reaction was completed, the mixture was purified by chromatography on silica gel (CH₂Cl₂/MeOH=10:1) to afford (Z)-N-(4-fluorophenethyl)-2-nitroethene-1,1-diamine (800 mg, 75.85%). LC-MS: m/z 226.1 (M+H)⁺.

### Step C: 4-(5-((3,4-difluorobenzyl)carbamoyl)thiophen-2-yl)-6-((4-fluorophenethyl)amino)-2-isobutyl-5-nitro-1,4-dihydropyridine-3-carboxamide

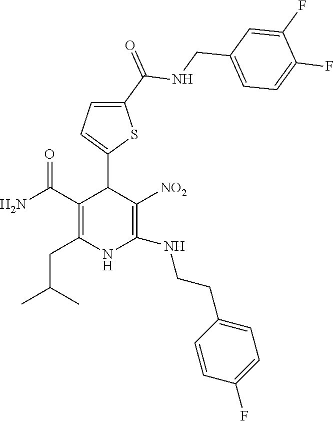 [SMILES: CC(C)CC1=C(C(N)=O)C(c2ccc(C(=O)NCc3ccc(F)c(F)c3)s2)C([N+](=O)[O-])=C(NCCc2ccc(F)cc2)N1]

A mixture of (Z)-N-(4-fluorophenethyl)-2-nitroethene-1,1-diamine (250 mg, 1.11 mmol) in EtOH (15 mL) were added 5-methyl-3-oxohexanamide (159 mg, 1.11 mmol), N-(3,4-difluorobenzyl)-5-formylthiophene-2-carboxamide (312 mg, 1.11 mmol), and the reaction mixture was stirred at 120° C. for 16 hours with N₂ protection in a sealed tube. The residue was concentrated and purified by reversed flash (0.1 FA/H₂O:ACN=0 to 51%) to afford 4-(5-((3,4-difluorobenzyl)carbamoyl)thiophen-2-yl)-6-((4-fluorophenethyl)amino)-2-isobutyl-5-nitro-1,4-dihydropyridine-3-carboxamide (290 mg, 42.6%). LC-MS: m/z 613.9 (M+H)⁺.

### Step D: 4-(5-((3,4-difluorobenzyl)carbamoyl)thiophen-2-yl)-6-((4-fluorophenethyl)amino)-2-isobutyl-5-nitronicotinamide

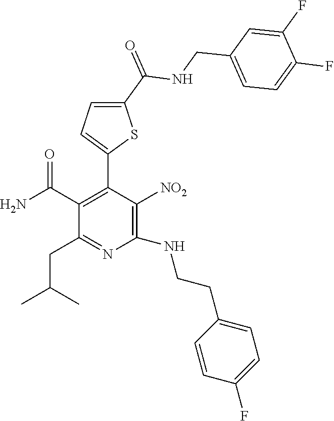 [SMILES: CC(C)Cc1nc(NCCc2ccc(F)cc2)c([N+](=O)[O-])c(-c2ccc(C(=O)NCc3ccc(F)c(F)c3)s2)c1C(N)=O]

A mixture of 4-(5-((3,4-difluorobenzyl)carbamoyl)thiophen-2-yl)-6-((4-fluorophenethyl)amino)-2-isobutyl-5-nitro-1,4-dihydropyridine-3-carboxamide (230 mg, 0.38 mmol) in DCM (10 mL) was added ceric ammonium nitrate (597 mg, 1.13 mmol) and the reaction mixture was stirred at 70° C. for 30 minutes in microwave. The reaction mixture was filtered, concentrated and purified by silica gel column chromatography eluting with (PE:EA=2:1) to afford 4-(5-((3,4-difluorobenzyl)carbamoyl)thiophen-2-yl)-6-((4-fluorophenethyl)amino)-2-isobutyl-5-nitronicotinamide (130 mg, 56.7%). LC-MS: m/z 612.1 (M+H)⁺.

### Step E: 5-amino-4-(5-((3,4-difluorobenzyl)carbamoyl)thiophen-2-yl)-6-((4-fluorophenethyl)amino)-2-isobutylnicotinamide

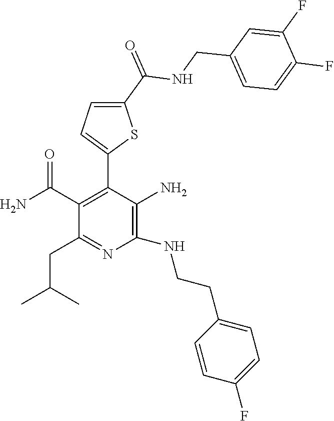 [SMILES: CC(C)Cc1nc(NCCc2ccc(F)cc2)c(N)c(-c2ccc(C(=O)NCc3ccc(F)c(F)c3)s2)c1C(N)=O]

A mixture of 4-(5-((3,4-difluorobenzyl)carbamoyl)thiophen-2-yl)-6-((4-fluorophenethyl)amino)-2-isobutyl-5-nitronicotinamide (130 mg, 0.21 mmol) in MeOH (8 mL) was added Pd/C and the reaction mixture was stirred at 40° C. for 2 hours with H₂ protection. The reaction mixture was filtered and concentrated in vacuum to afford 5-amino-4-(5-((3,4-difluorobenzyl)carbamoyl)thiophen-2-yl)-6-((4-fluorophenethyl)amino)-2-isobutylnicotinamide (45 mg, 36.4%). LC-MS: m/z 582.1 (M+H)⁺.

### Step F: 7-(5-{[(3,4-difluorophenyl)methyl]carbamoyl}-2-thienyl)-3-[2-(p-fluorophenyl)ethyl]-5-isobutyl-3H-1,3,4-triazaindene-6-carboxamide (Compound 132)

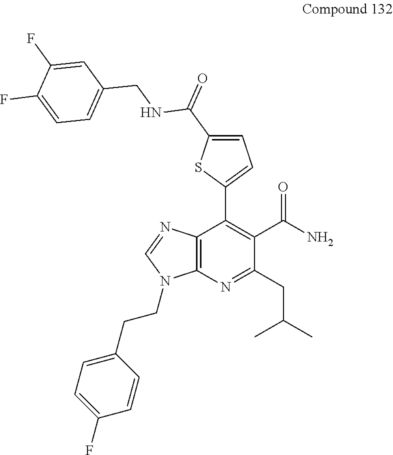 [SMILES: CC(C)Cc1nc2c(ncn2CCc2ccc(F)cc2)c(-c2ccc(C(=O)NCc3ccc(F)c(F)c3)s2)c1C(N)=O]

A mixture of 5-amino-4-(5-((3,4-difluorobenzyl)carbamoyl)thiophen-2-yl)-6-((4-fluorophenethyl)amino)-2-isobutylnicotinamid (40 mg, 0.069 mmol) in triethyl orthoformate (1 mL) was stirred at 120° C. for 20 minutes in a sealed tube. After the reaction was completed, the mixture was purified by Prep-HPLC (0.1% FA) to afford 7-(5-{[(3,4-difluorophenyl)methyl]carbamoyl}-2-thienyl)-3-[2-(p-fluorophenyl)ethyl]-5-isobutyl-3H-1,3,4-triazaindene-6-carboxamide (8.05 mg, 19.8%).

¹H NMR (400 MHz, CD₃OD) δ 8.09 (s, 1H), 7.73 (d, J=4.0 Hz, 1H), 7.60 (d, J=4.0 Hz, 1H), 7.17-7.28 (m, 3H), 7.05-7.08 (m, 2H), 6.90-6.94 (m, 2H), 4.61 (1, 6.8 Hz, 2H), 4.52 (s, 2H), 3.23-3.27 (m, 2H), 2.88 (d, J=7.2 Hz, 2H), 2.37-2.44 (m, 1H), 1.00 (d, J=6.8 Hz, 6H). ¹⁹F NMR (376 MHz, CD₃OD) δ −118.33, −140.66, −143.14. LC-MS: m/z 592.0 (M+H)⁺.

### 7-[(R)-6-fluoro-1-indanylamino]-1′-[(p-fluorophenyl)methyl]-6′-isobutyl-1′H-1-thia-1′,3′,6,7′-tetraaza-[2,4′-biindenyl]-5′-carboxamide (Compound 133)

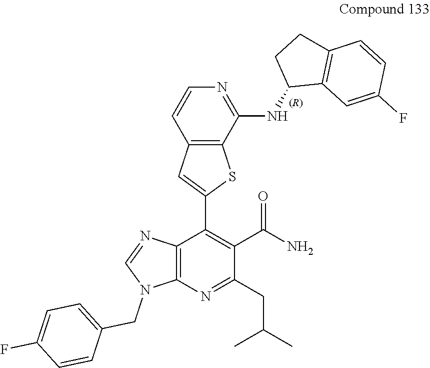

Compound 133 was synthesized following the similar route of Example 19, using 7-chlorothieno[2,3-c]pyridine-2-carbaldehyde in step C, and then following the similar route of Example 1, using 7-(7-chlorothieno[2,3-c]pyridin-2-yl)-3-(4-fluorobenzyl)-5-isobutyl-3H-imidazo[4,5-b]pyridine-6-carboxamide and (R)-6-fluoro-2,3-dihydro-1H-inden-1-amine in step J.

¹H NMR (400 MHz, DMSO-d₆) δ 8.49 (s, 2H), 7.52 (d, J=5.6 Hz, 1H), 7.77 (s, 1H), 7.46 (q, J=5.2 Hz, 2H), 7.28 (q, J=5.2 Hz, 1H), 7.15 (d, J=6.0 Hz, 1H), 7.05 (t, J=8.4 Hz, 2H), 6.89-6.94 (m, 2H), 5.85 (t, J=8.0 Hz, 1H), 5.55 (s, 2H), 4.80 (s, 1H), 2.98-2.99 (m, 1H), 2.91 (d, J=7.2 Hz, 2H), 2.66-2.74 (m, 1H), 2.42 (p, J=7.2 Hz, 1H), 2.02-2.10 (m, 1H), 0.91 (d, J=6.8 Hz, 6H). ¹⁹F NMR (376 MHz, DMSO-d₆) δ −116.14, −119. LC-MS: m/z 609.3 (M+H)⁺.

### Example 20

### 7-{5-[N-(R)-1-indanylcarbamoyl]-2-thienyl}-2-[2-(p-fluorophenyl)ethyl]-5-isobutyl-1-oxo-2,4-diaza-6-indancarboxamide (Compound 134)

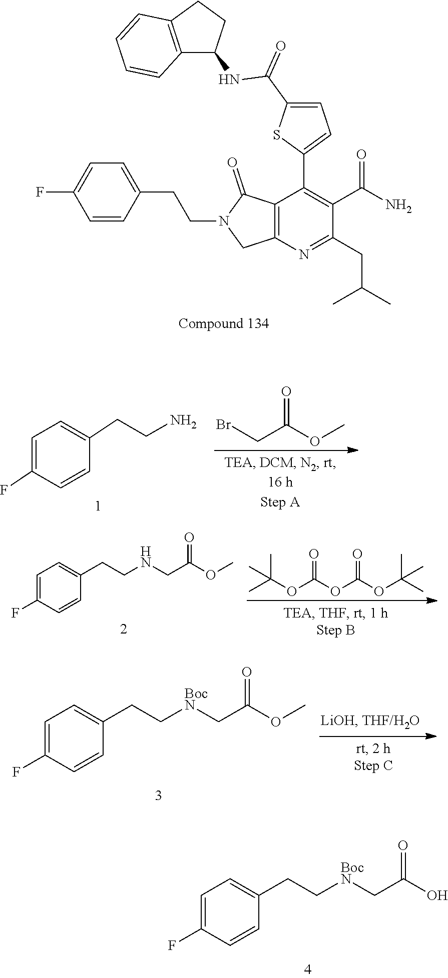

### Step A: methyl(4-fluorophenethyl)glycinate

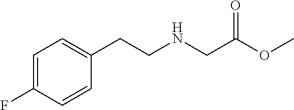

To a solution of 2-(4-fluorophenyl) ethan-1-amine (2 g, 14.39 mmol) in THF (20 ml.) was added methyl 2-bromoacetate (2.2 g, 14.39 mmol) and TEA (2.78 g, 21.59 mmol), the mixture was stirred at room temperature for 16 hours. The reaction mixture was concentrated in vacuo to afford methyl(4-fluorophenethyl)glycinate as a crude which was used directly in the next step without further purification. LC-MS: m/z 212.1 (M+H)⁺.

### Step B: methyl N-(tert-butoxycarbonyl)-N-(4-fluorophenethyl)glycinate

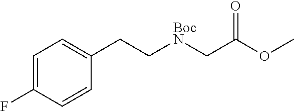 [SMILES: COC(=O)CN(CCc1ccc(F)cc1)C(=O)OC(C)(C)C]

To a solution of methyl(4-fluorophenethyl)glycinate (crude) in THF (20 mL) were added di-tert-butyl dicarbonate (4.65 g, 21.33 mmol) and TEA (1.87 g, 18.49 mmol), the mixture was stirred at room temperature for 1 hour. The reaction mixture was diluted with DCM and aq. NaCl. The organic layer was separated, washed with brine, concentrated in vacuo and purified by silica gel chromatography (EA:PE=0 to 20%) to afford methyl N-(tert-butoxycarbonyl)-N-(4-fluorophenethyl)glycinate (2.95 g, 62.5%). LC-MS: m/z 212.0 (M+H-Boc)⁺.

### Step C: N-(tert-butoxycarbonyl)-N-(4-fluorophenethyl)glycine

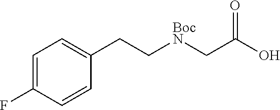 [SMILES: CC(C)(C)OC(=O)N(CCc1ccc(F)cc1)CC(=O)O]

To a solution of methyl N-(tert-butoxycarbonyl)-N-(4-fluorophenethyl)glycinate (2.95 g, 8.04 mmol) in THF (30 mL) and H₂O (10 mL) was added LiOH (1.69 g, 40.19 mmol), the mixture was stirred at room temperature for 2 hour. The reaction mixture were diluted with DCM (40 mL) and adjusted pH to 5-6 by the addition of HCl (1 M) solution slowly, followed the DCM were washed with H₂O (30 mL×3), dried, filtered, and concentrated in vacuo to afford N-(tert-butoxycarbonyl)-N-(4-fluorophenethyl)glycine (2.8 g, crude) which was used directly in the next step without further purification. LC-MS: m/z 198.1 (M+H-Boc)⁺.

### 7-{5-[N-(R)-1-indanylcarbamoyl]-2-thienyl}-2-[2-(p-fluorophenyl)ethyl]-5-isobutyl-1-oxo-2,4-diaza-6-indancarboxamide (Compound 134)

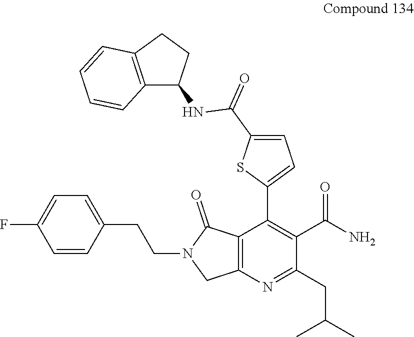 [SMILES: CC(C)Cc1nc2c(c(-c3ccc(C(=O)N[C@@H]4CCc5ccccc54)s3)c1C(N)=O)C(=O)N(CCc1ccc(F)cc1)C2]

Compound 134 was then synthesized following the similar route of Example 10, using N-(tert-butoxycarbonyl)-N-(4-fluorophenethyl)glycine in step C.

¹H NMR (400 MHz, DMSO-d₆) δ 8.88 (d, J=8.4 Hz, 1H), 7.84 (s, 1H), 7.77-7.78 (m, 1H), 7.60 (s, 1H), 7.21-7.30 (m, 6H), 7.09-7.14 (m, 3H), 5.49-5.57 (m, 1H), 4.45 (s, 2H), 3.71 (t, J=7.6 Hz, 2H), 2.97-3.04 (m, 1H), 2.85-2.93 (m, 3H), 2.72-2.74 (m, 2H), 2.44-2.47 (m, 1H), 2.24-2.33 (m, 1H), 1.93-2.03 (m, 1H), 0.91-0.92 (m, 6H). ¹⁹F NMR (376 MHz, DMSO-d₆) δ −116.81. LC-MS: m/z 597.1 (M+H)⁺.

### 4-{5-[N-(R)-1-indanylcarbamoyl]-2-thienyl}-8-[(p-fluorophenyl)methyl]-2-isobutyl-5-oxo-5,6,7,8-tetrahydro-1,6-diaza-3-naphthamide (Compound 135)

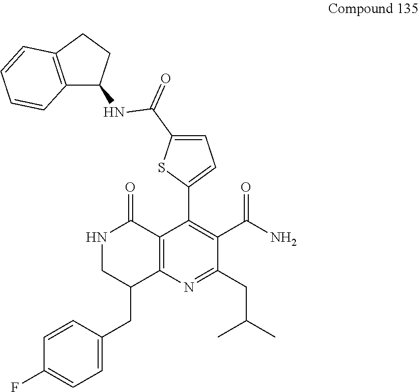 [SMILES: CC(C)Cc1nc2c(c(-c3ccc(C(=O)N[C@@H]4CCc5ccccc54)s3)c1C(N)=O)C(=O)NCC2Cc1ccc(F)cc1]

Compound 135 was synthesized following the similar route of Example 10, using 2-(2,2-dimethyl-4-oxo-5-aza-3-oxahex-6-yl)-3-(4-fluorophenyl) propanoic acid in step C.

¹H NMR (400 MHz, DMSO-d₆) δ 8.80 (d, J=8.4 Hz, 1H), 7.93-7.99 (m, 1H), 7.77 (s, 1H), 7.70 (d, J=3.6 Hz, 1H), 7.49 (s, 1H), 7.20-7.36 (m, 6H), 7.10-7.19 (m, 2H), 6.94 (d, J=4.0 Hz, 1H), 5.51 (q, J=8.0 Hz, 1H), 3.30-3.35 (m, 1H), 3.22-3.27 (m, 1H), 3.15-3.22 (m, 1H), 2.96-3.12 (m, 2H), 2.84-2.91 (m, 1H), 2.74-2.80 (m, 1H), 2.61-2.67 (m, 2H), 2.40-2.47 (m, 1H), 2.16-2.25 (m, 1H), 1.91-2.02 (m, 1H), 0.85-0.94 (m, 6H). ¹⁹F NMR (376 MHz, DMSO-d₆) δ −116.92. LC-MS: m/z 597.1 (M+H)⁺.

### Example 21

### N-[(R)-1-indanyl]-5-{6-carbamoyl-3-[(p-fluorophenyl)methyl]-5-isobutyl-4-aza-7-indanyl}-2-thenamide (Compound 136)

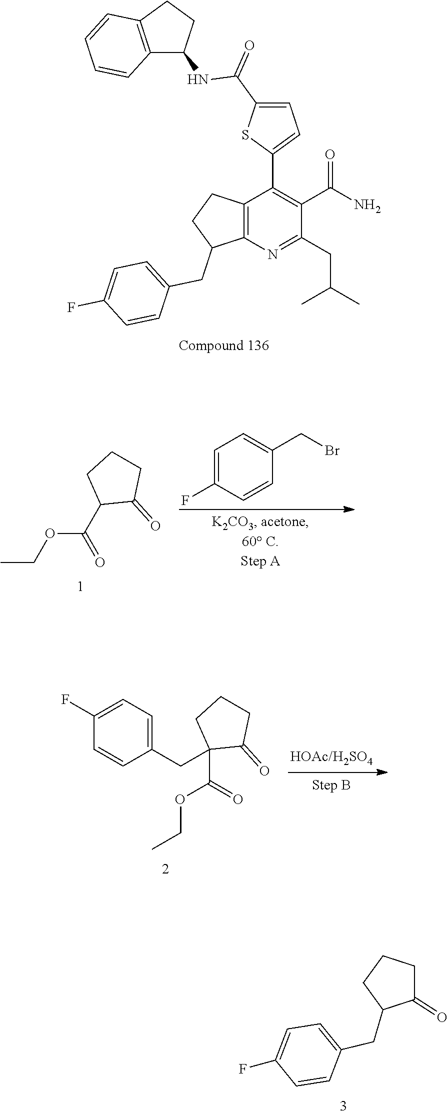

### Step A: ethyl 1-[(4-fluorophenyl)methyl]-2-oxocyclopentane-1-carboxylate

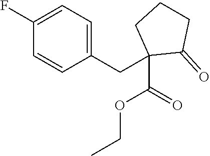 [SMILES: CCOC(=O)C1(Cc2ccc(F)cc2)CCCC1=O]

To a solution of ethyl 2-oxocyclopentane-1-carboxylate (10 g, 64.029 mmol) in acetone (10 mL) were added 1-(bromomethyl)-4-fluorobenzene (18.16 g, 96.043 mmol) and K₂CO₃ (26.55 g, 192.086 mmol). The reaction mixture was stirred at 60° C. for 4 hours. The reaction mixture was filtered and concentrated in vacuo. The residue was purified by silica gel column chromatography eluting with (PE:EA=5:1) to afford ethyl 1-[(4-fluorophenyl)methyl]-2-oxocyclopentane-1-carboxylate (15 g, 88.64%). LC-MS: m/z 265.1 (M+H)⁺.

### Step B: 2-[(4-fluorophenyl)methyl]cyclopentan-1-one

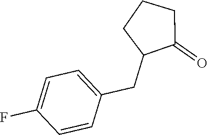 [SMILES: O=C1CCCC1Cc1ccc(F)cc1]

A solution of ethyl 1-[(4-fluorophenyl)methyl]-2-oxocyclopentane-1-carboxylate (15 g, 56.754 mmol) in H₂SO₄ (50 mL, 12.5% in water) and HOAc (100 mL) was stirred at 120° C. for 3 hours. The reaction mixture was concentrated in vacuo and the residue was diluted with EA (200 mL) and water (100 mL). The organic layer was separated, washed with water (100 mL×2) and saturated NaCl (100 mL). The organic layer was separated, dried over Na₂SO₄ and then filtered. The organic layer was collected, concentrated in vacuo. The residue was purified by silica gel column chromatography eluting with PE:EA=5:1 to afford 2-[(4-fluorophenyl)methyl]cyclopentan-1-one (9.7 g, 88.91%). LC-MS: m/z 193.2 (M+H)⁺.

### N-[(R)-1-indanyl]-5-{6-carbamoyl-3-[(p-fluorophenyl)methyl]-5-isobutyl-4-aza-7-indanyl}-2-thenamide (Compound 136)

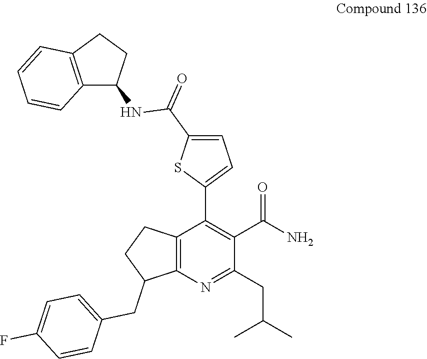 [SMILES: CC(C)Cc1nc2c(c(-c3ccc(C(=O)N[C@@H]4CCc5ccccc54)s3)c1C(N)=O)CCC2Cc1ccc(F)cc1]

Compound 136 was then synthesized following the similar route of Example 10, using 2-[(4-fluorophenyl)methyl]cyclopentan-1-one in step D.

¹H NMR (400 MHz, DMSO-d₆) δ 8.87 (d, J=4.4 Hz, 1H), 7.75-7.82 (m, 2H), 7.48 (s, 1H), 7.17-7.33 (m, 7H), 7.10 (t, J=8.8 Hz, 2H), 5.47-5.56 (m, 1H), 3.14-3.22 (m, 2H), 2.99-3.04 (m, 1H), 2.86-2.95 (m, 1H), 2.75-2.83 (m, 2H), 2.62-2.69 (m, 3H), 2.41-2.43 (m, 1H), 2.21-2.30 (m, 1H), 1.92-2.11 (m, 2H), 1.64-1.75 (m, 1H), 0.92 (d, J=6.8 Hz, 3H), 0.91 (d, J=6.8 Hz, 3H). ¹⁹F NMR (376 MHz, DMSO-d₆) δ −117.44. LC-MS: m/z 568.2 (M+H)⁺.

### Example 24

### (S)-7-{3-[(3,4-difluorophenyl)methyl]-1,3a-diaza-6-indenyl}-3-[(p-fluorophenyl)methyl]-5-isobutyl-3-methyl-1-oxo-2,4-diaza-6-indancarboxamide (Compound 141)

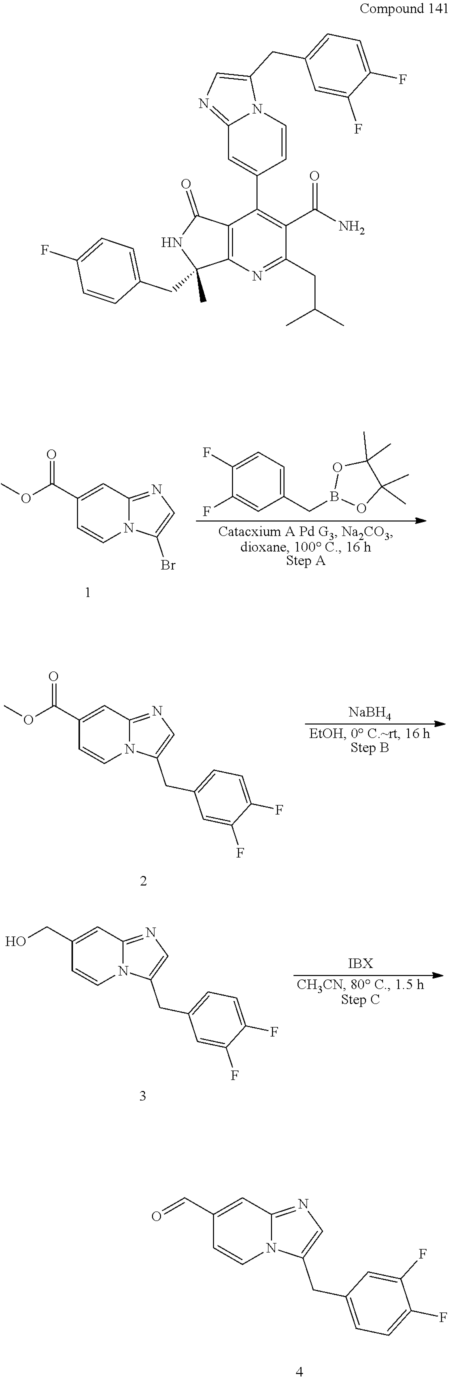

### Step A: methyl 3-(3,4-difluorobenzyl)imidazo[1,2-a]pyridine-7-carboxylate

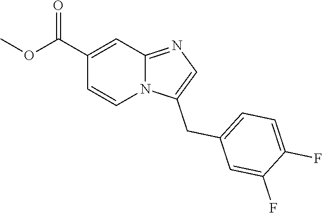 [SMILES: COC(=O)c1ccn2c(Cc3ccc(F)c(F)c3)cnc2c1]

A mixture of methyl 3-bromoimidazo[1,2-a]pyridine-7-carboxylate (200 mg, 0.784 mmol), 2-(3,4-difluorobenzyl)-4,4,5,5-tetramethyl-1,3,2-dioxaborolane (398.45 mg, 1.568 mmol), cataCXium A Pd G3 (56.78 mg, 0.078 mmol) and Na₂CO₃ (2 mL) in dioxane (4 mL) was stirred at 100° C. for 16 hours under N₂ atmosphere. The reaction mixture was poured into water (50 mL) and extracted with EA (2×50 mL), the combined organic layer was washed with brine, dried over anhydrous Na₂SO₄, filtered and the residue was purified by reverse-phase flash column chromatography (1.0% TFA in the mixture of ACN and water) to afford methyl 3-(3,4-difluorobenzyl)imidazo[1,2-a]pyridine-7-carboxylate (220 mg, 92.82%). LC-MS: m/z 303.2 (M+H)⁺.

### Step B: (3-(3,4-difluorobenzyl)imidazo[1,2-a]pyridin-7-yl) methanol

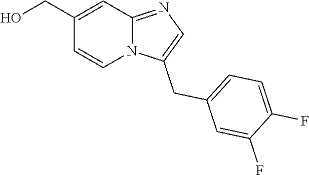 [SMILES: OCc1ccn2c(Cc3ccc(F)c(F)c3)cnc2c1]

A mixture of methyl 3-(3,4-difluorobenzyl)imidazo[1,2-a]pyridine-7-carboxylate (220 mg, 0,728 mmol) in EtOH (10 mL) was added NaBH₄ (137.66 mg, 3.639 mmol) at 0° C. The mixture was stirred at room temperature for 16 hours under N₂ atmosphere. The mixture was poured into aq. NH₄Cl (100 mL) and extracted with EA (2×100 mL), the combined organic layer was washed with brine, dried over anhydrous Na₂SO₄, filtered and the residue was purified by flash column chromatography (PE/EtOAc=30/1) to afford (3-(3,4-difluorobenzyl)imidazo[1,2-a]pyridin-7-yl) methanol (62 mg, 31.06%). LC-MS: m/z 275.2 (M+H)⁺.

### Step C: 3-(3,4-difluorobenzyl)imidazo[1,2-a]pyridine-7-carbaldehyde

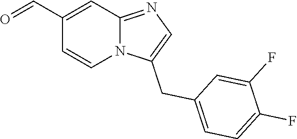 [SMILES: O=Cc1ccn2c(Cc3ccc(F)c(F)c3)cnc2c1]

A mixture of (3-(3,4-difluorobenzyl)imidazo[1,2-a]pyridin-7-yl) methanol (62 mg, 0.226 mmol) and IBX (126.59 mg, 0.452 mmol) in ACN (5 mL) was stirred at 80° C. for 1.5 hours under N₂ atmosphere. The mixture was filtered and the concentrated to afford 3-(3,4-difluorobenzyl)imidazo[1,2-a]pyridine-7-carbaldehyde (72 mg, crude) which was used in the next step without further purification. LC-MS: m/z 273.3 (M+H)⁺.

### (S)-7-{3-[(3,4-difluorophenyl)methyl]-1,3a-diaza-6-indenyl}-3-[(p-fluorophenyl)methyl]-5-isobutyl-3-methyl-1-oxo-2,4-diaza-6-indancarboxamide (Compound 141)

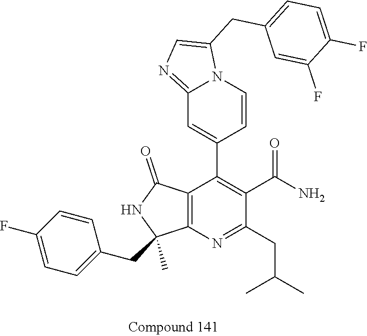 [SMILES: CC(C)Cc1nc2c(c(-c3ccn4c(Cc5ccc(F)c(F)c5)cnc4c3)c1C(N)=O)C(=O)N[C@]2(C)Cc1ccc(F)cc1]

Compound 141 was then synthesized following the similar route of Example 2, using 3-(3,4-difluorobenzyl)imidazo[1,2-a]pyridine-7-carbaldehyde in step B.

¹H NMR (400 MHz, CD₃OD) δ 8.04 (d, J=7.2 Hz, 1H), 7.44 (s, 2H), 7.15-7.22 (m, 2H), 7.05-7.08 (m, 1H), 6.79-6.89 (m, 4H), 6.60-6.70 (m, 1H), 4.30 (s, 2H), 3.40 (d, J=13.6 Hz, 1H), 3.16 (d, J=13.6 Hz, 1H), 2.89-2.95 (m, 2H), 2.41-2.51 (m, 1H), 1.68 (s, 3H), 1.08 (d, J=6.4 Hz, 3H), 1.01 (d, J=6.8 Hz, 3H). ¹⁹F NMR (376 MHz, CD₃OD) δ −118.23, −140.19, −143.45. LC-MS: m/z 598.1 (M+H)⁺.

### Example 25

### (S)-7-{3-[(3,4-difluorophenyl)methyl]-2-oxo-1,3-benzoxazol-6-yl}-3-[(p-fluorophenyl)methyl]-5-isobutyl-3-methyl-1-oxo-2,4-diaza-6-indancarboxamide (Compound 142)

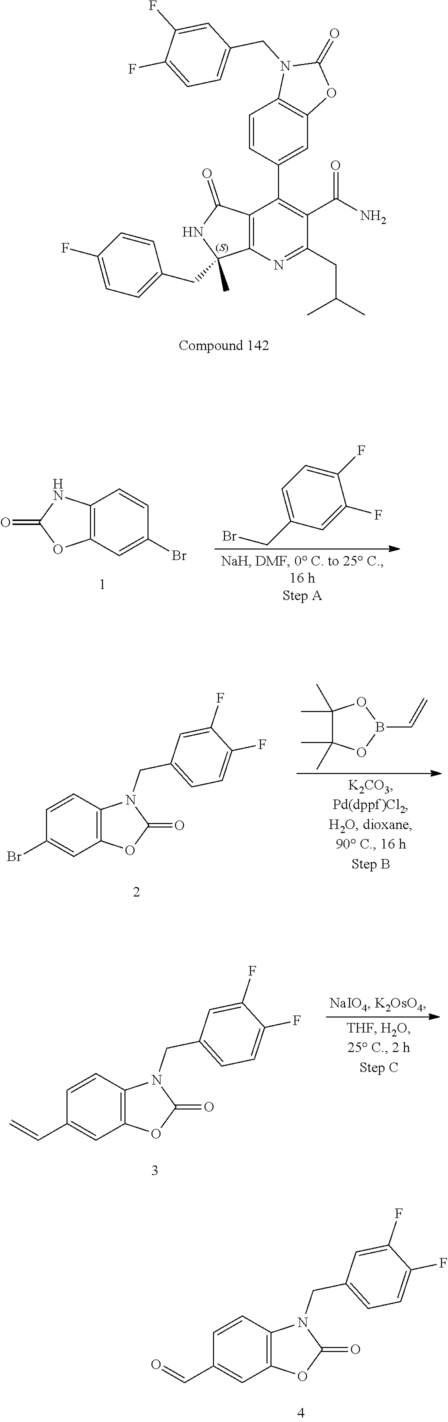

### Step A: 6-bromo-3-(3,4-difluorobenzyl)benzo[d]oxazol-2 (3H)-one

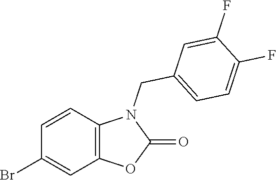 [SMILES: O=c1oc2cc(Br)ccc2n1Cc1ccc(F)c(F)c1]

To a solution of 6-bromobenzo[d]oxazol-2 (3H)-one (1000 mg, 4.672 mmol) in DMF (20 mL) was added NaH (224.26 mg, 9.344 mmol) at 0° C., the reaction mixture was stirred at 0° C. for 30 mins under N₂. Then 4-(bromomethyl)-1,2-difluorobenzene (2901.59 mg, 14.016 mmol) was added. The resulting mixture was stirred at 25° C. for 18 hours under N₂. To the mixture was added water (30 mL), the mixture was extracted with EA (30 mL×2), the combined organic layers were washed with brine (30 mL×3), dried over anhydrous Na₂SO₄, filtered and concentrated under vacuum. The resulting residue was purified by silica gel chromatography (eluting with EA/PE=1/5) to afford 6-bromo-3-(3,4-difluorobenzyl)benzo[d]oxazol-2 (3H)-one (1300 mg, 81.81%).

¹H NMR (400 MHz, CDCl₃) δ 7.39 (d, J=1.6 Hz, 1H), 7.25-7.27 (m, 1H), 7.12-7.19 (m, 2H), 7.05-7.08 (m, 1H), 6.69 (d, J=8.4 Hz, 1H), 4.93 (s, 2H).

### Step B: 3-(3,4-difluorobenzyl)-6-vinylbenzo[d]oxazol-2 (3H)-one

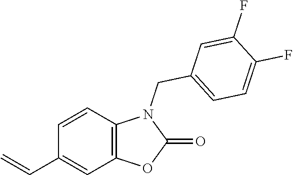 [SMILES: C=Cc1ccc2c(c1)oc(=O)n2Cc1ccc(F)c(F)c1]

To a solution of 6-bromo-3-(3,4-difluorobenzyl)benzo[d]oxazol-2 (3H)-one (650 mg, 1.911 mmol) and 4,4,5,5-tetramethyl-2-vinyl-1,3,2-dioxaborolane (353.22 mg, 2.293 mmol) in dioxane (20 mL) and H₂O (2 mL) were added K₂CO₃ (792.34 mg, 5.733 mmol) and Pd(dppf)Cl₂ (139.84 mg, 0.191 mmol) under N₂. The reaction mixture was stirred at 80° C. for 18 hours. The reaction mixture was concentrated under vacuum. The resulting residue was purified by silica gel column chromatography (eluting with PE/EA=5/1) to afford 3-(3,4-difluorobenzyl)-6-vinylbenzo[d]oxazol-2 (3H)-one (400 mg, 72.86%). LC-MS: m/z 288.1 (M+H)⁺.

### Step C: 3-(3,4-difluorobenzyl)-2-oxo-2,3-dihydrobenzo[d]oxazole-6-carbaldehyde

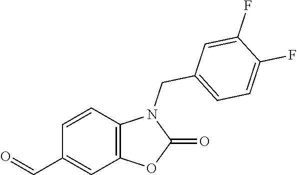 [SMILES: O=Cc1ccc2c(c1)oc(=O)n2Cc1ccc(F)c(F)c1]

To a solution of 3-(3,4-difluorobenzyl)-6-vinylbenzo[d]oxazol-2 (3H)-one (200 mg, 0.696 mmol) in THF (5 mL) and H₂O (5 mL) was added NaIO₄ (744.56 mg, 3.481 mmol). Then potassium osmate(VI) dihydrate (4.34 mg, 0.014 mmol) was added at 0° C. The reaction mixture was stirred at 25° C. for 2 hours under N₂. To the mixture was added water (30 mL), the resulting mixture was extracted with EA (30 mL×2), the combined organic layers were washed with brine (30 mL×3), dried over anhydrous Na₂SO₄, filtered and concentrated under vacuum. The resulting residue was purified by silica gel chromatography (eluting EA/PE=1/2) to afford 3-(3,4-difluorobenzyl)-2-oxo-2,3-dihydrobenzo[d]oxazole-6-carbaldehyde (110 mg, 54.63%).

¹H NMR (400 MHz, DMSO-d₆) δ 9.93 (s, 1H) 7.82-7.84 (m, 2H), 7.53-7.58 (m, 1H), 7.43-7.45 (m, 2H), 7.26-7.29 (m, 1H), 5.10 (s, 2H).

### (S)-7-{3-[(3,4-difluorophenyl)methyl]-2-oxo-1,3-benzoxazol-6-yl}-3-[(p-fluorophenyl)methyl]-5-isobutyl-3-methyl-1-oxo-2,4-diaza-6-indancarboxamide (Compound 142)

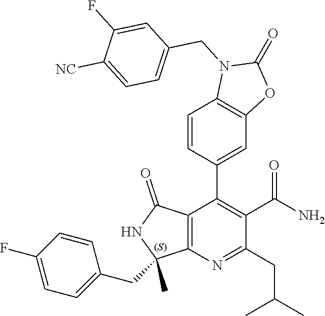

Compound 142 was then synthesized following the similar route of Example 2, using 3-(3,4-difluorobenzyl)-2-oxo-2,3-dihydrobenzo[d]oxazole-6-carbaldehyde in step B.

¹H NMR (400 MHz, DMSO-d₆) δ 8.71 (s, 1H), 7.70 (s, 1H), 7.51-7.59 (m, 1H), 7.41-7.49 (m, 2H), 7.24-7.29 (m, 2H), 7.02-7.08 (m, 1H), 6.82-6.91 (m, 5H), 5.03 (s, 2H), 3.19-3.24 (m, 1H), 3.06-3.09 (m, 1H), 2.75 (m, 2H), 2.36 (m, 1H), 1.55 (s, 3H), 0.91-1.01 (m, 6H). ¹⁹F NMR (376 MHz DMSO-d₆) δ −116.68, −137.81, −139.39. LC-MS: m/z 615.2 (M+H)⁺.

### Example 26

### (S)-7-{3-[(5-cyano-2-pyridyl)methyl]-2-oxo-1,3-benzoxazol-6-yl}-3-[(p-fluorophenyl)methyl]-5-isobutyl-3-methyl-1-oxo-2,4-diaza-6-indancarboxamide (Compound 143)

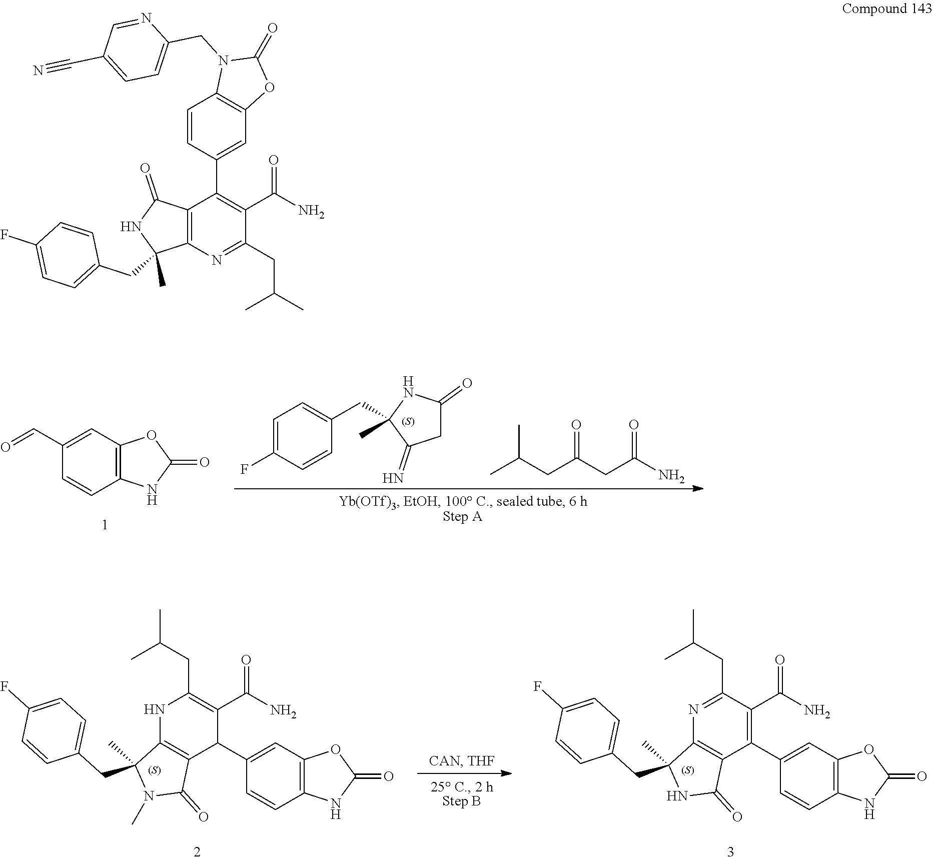

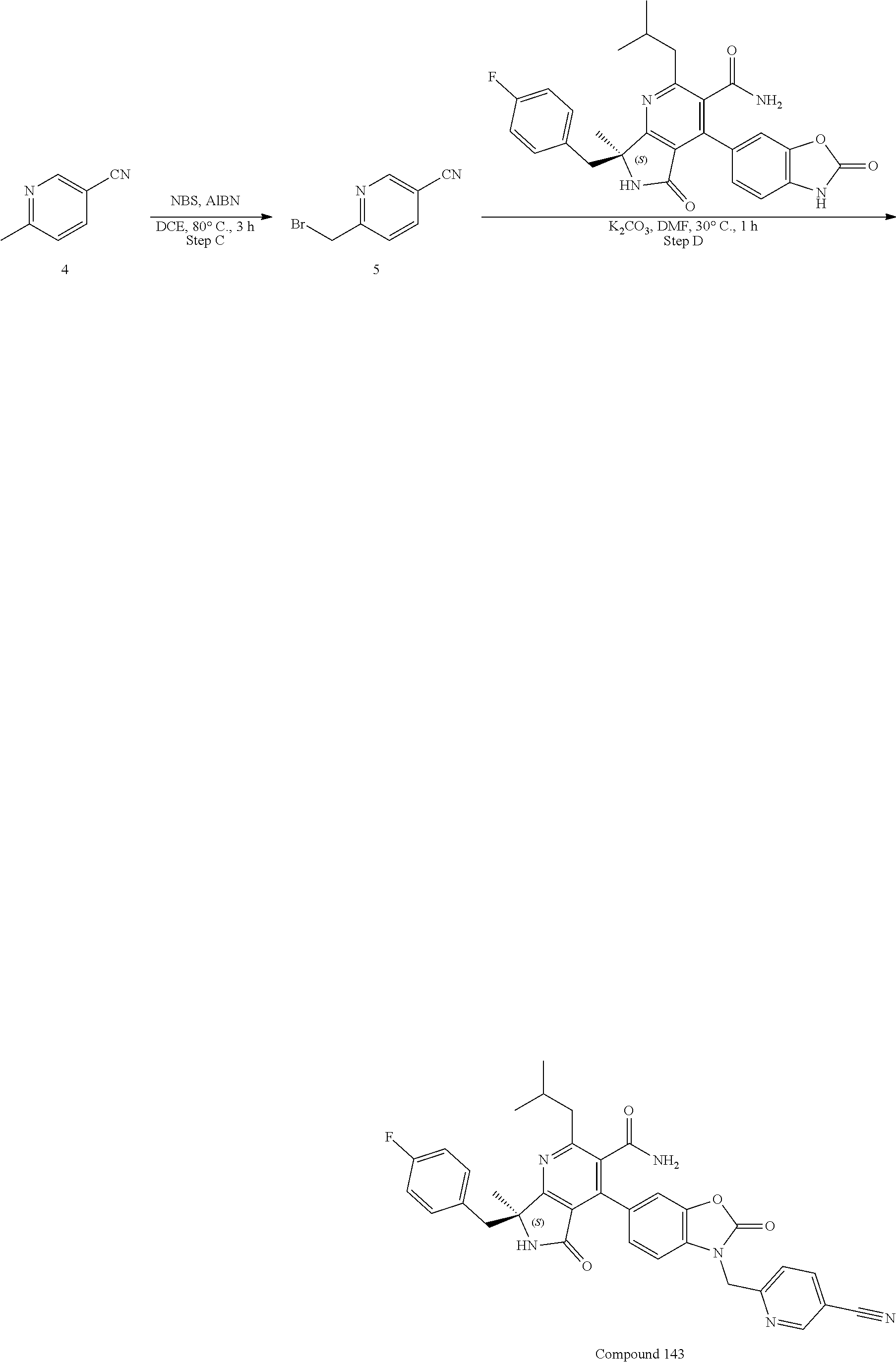

### Step A: (7S)-7-(4-fluorobenzyl)-2-isobutyl-7-methyl-5-oxo-4-(2-oxo-2,3-dihydrobenzo[d]oxazol-6-yl)-4,5,6,7-tetrahydro-1H-pyrrolo[3,4-b]pyridine-3-carboxamide

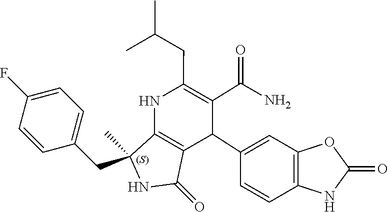

To a solution of 2-oxo-3H-benzo[d][1,3]oxazole-6-carbaldehyde (250 mg, 1.533 mmol) in EtOH (5 mL) were added(S)-4-amino-5-(4-fluorobenzyl)-5-methyl-1,5-dihydro-2H-pyrrol-2-one (50 mg, 0.173 mmol), Yb(OTf)₃ (63.37 mg, 0.102 mmol) and 5-methyl-3-oxohexanamide (438.88 mg, 3.065 mmol), the reaction mixture was stirred at 100° C. for 6 hours in a sealed tube under N₂. The reaction mixture was concentrated under vacuum to afford (7S)-7-(4-fluorobenzyl)-2-isobutyl-7-methyl-5-oxo-4-(2-oxo-2,3-dihydrobenzo[d]oxazol-6-yl)-4,5,6,7-tetrahydro-1H-pyrrolo[3,4-b]pyridine-3-carboxamide (500 mg, 99.77%), which was used in the next step without further purification. LC-MS: m/z 491.4 (M+H)⁺.

### Step B: (S)-7-(4-fluorobenzyl)-2-isobutyl-7-methyl-5-oxo-4-(2-oxo-2,3-dihydrobenzo[d]oxazol-6-yl)-6,7-dihydro-5H-pyrrolo[3,4-b]pyridine-3-carboxamide

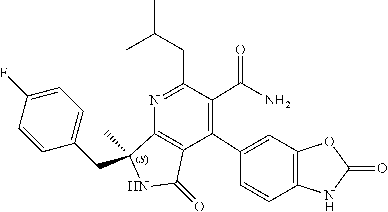

To a solution of (7S)-7-(4-fluorobenzyl)-2-isobutyl-7-methyl-5-oxo-4-(2-oxo-2,3-dihydrobenzo[d]oxazol-6-yl)-4,5,6,7-tetrahydro-1H-pyrrolo[3,4-b]pyridine-3-carboxamide (500 mg, 1.019 mmol) in THF (10 mL) was added ceric ammonium nitrate (1117.58 mg, 2.039 mmol) under N₂. The reaction mixture was stirred at 25° C. for 2 hours under N₂. To the reaction mixture was added. aq. NaHCO₃ until pH=8, the mixture was extracted with EA (50 mL×3). The combined organics was washed with brine (50 mL) and dried over anhydrous Na₂SO₄, filtered and concentrated under vacuum. The resulting residue was purified by prep-TLC (eluting DCM/MeOH=10/1) to afford (S)-7-(4-fluorobenzyl)-2-isobutyl-7-methyl-5-oxo-4-(2-oxo-2,3-dihydrobenzo[d]oxazol-6-yl)-6,7-dihydro-5H-pyrrolo[3,4-b]pyridine-3-carboxamide (450 mg, 90.37%). LC-MS: m/z 489.0 (M+H)⁺.

### Step C: 3-(3,4-difluorobenzyl)-2-oxo-2,3-dihydrobenzo[d]oxazole-6-carbaldehyde

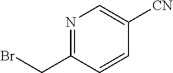

To a solution of 6-methylnicotinonitrile (2 g, 16.942 mmol) in tetrachloromethane (15 mL) were added NBS (3.31 g, 18.622 mmol) and AIBN (0.28 g, 1.693 mmol), the reaction mixture was stirred at 85° C. for 2 hours under N₂. To the mixture was added water (20 mL), the resulting mixture was extracted with DCM (20 mL), the organic layer was washed with aq. NaHCO₃ (20 mL) and brine (20 mL), dried over anhydrous Na₂SO₄, filtered and concentrated in vacuum. The residue was purified by silica gel chromatography (eluting PE/EA=25/1 to 15/1) to afford 6-(bromomethyl) nicotinonitrile (1640 mg, 49.40%). LC-MS: m/z 196.9 (M+H)⁺.

### Step D: (S)-7-{3-[(5-cyano-2-pyridyl)methyl]-2-oxo-1,3-benzoxazol-6-yl}-3-[(p-fluorophenyl)methyl]-5-isobutyl-3-methyl-1-oxo-2,4-diaza-6-indancarboxamide

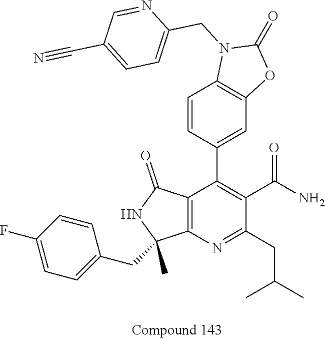 [SMILES: CC(C)Cc1nc2c(c(-c3ccc4c(c3)oc(=O)n4Cc3ccc(C#N)cn3)c1C(N)=O)C(=O)N[C@@]2(C)Cc1ccc(F)cc1]

To a solution of(S)-7-(4-fluorobenzyl)-2-isobutyl-7-methyl-5-oxo-4-(2-oxo-2,3-dihydrobenzo[d]oxazol-6-yl)-6,7-dihydro-5H-pyrrolo[3,4-b]pyridine-3-carboxamide (210 mg, 0.430 mmol) in DMF (15 mL) were added 6-(bromomethyl)pyridine-3-carbonitrile (211.74 mg, 1.075 mmol) and K₂CO₃ (297.04 mg, 2.149 mmol), and the mixture was stirred at 30° C. for 1 hour under N₂. To the mixture was added water (50 mL), the resulting mixture was extracted with EA (50 mL×2), the combined organic layers were washed with brine (20 mL×3), dried over anhydrous Na₂SO₄, filtered and concentrated under vacuum. The resulting residue was purified by silica gel chromatography (eluting DCM/MeOH=50/1) to afford a crude product (240 mg), which was then purified by prep-HPLC (Waters 3767/Qda Column: SunFire Sunfire C18, 19*250 mm*10 μm; Mobile Phase A: 0.1% FA/H₂O, B: ACN; Flow rate: 40 mL/min; Gradient: 46%; Retention Time: 9.2-10.1 min of 17 min) to afford (S)-7-{3-[(5-cyano-2-pyridyl)methyl]-2-oxo-1,3-benzoxazol-6-yl}-3-[(p-fluorophenyl)methyl]-5-isobutyl-3-methyl-1-oxo-2,4-diaza-6-indancarboxamide (166 mg, 63.62%).

¹H NMR (400 MHz, DMSO-d₆) δ 8.96 (d, J=1.6 Hz, 1H), 8.71 (s, 1H), 8.35 (dd, J=8.4, 2.0 Hz, 1H), 7.74 (d, J=8.4 Hz, 1H), 7.71 (s, 1H), 7.45 (s, 1H), 7.14 (d, J=8.0 Hz, 1H), 7.07 (m, 1H), 6.89-6.93 (m, 3H), 6.81-6.85 (m, 2H), 5.30 (s, 2H), 3.23 (d, J=13.6 Hz, 1H), 3.05 (d, J=13.6 Hz, 1H), 2.71-2.82 (m, 2H), 2.32-2.41 (m, 1H), 1.55 (s, 3H), 1.02 (d, J=6.4 Hz, 3H), 0.93 (d, J=6.4 Hz, 3H). ¹⁹F NMR (376 MHz DMSO-d₆) δ −116.69. LC-MS: m/z 605.6 (M+H)⁺.

### (S)-3-[(p-fluorophenyl)methyl]-7-{3-[(5-fluoro-2-pyridyl)methyl]-2-oxo-1,3-benzoxazol-6-yl}-5-isobutyl-3-methyl-1-oxo-2,4-diaza-6-indancarboxamide (Compound 144)

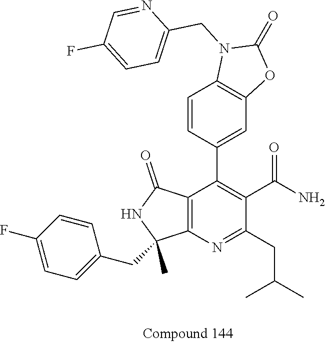 [SMILES: CC(C)Cc1nc2c(c(-c3ccc4c(c3)oc(=O)n4Cc3ccc(F)cn3)c1C(N)=O)C(=O)N[C@@]2(C)Cc1ccc(F)cc1]

Compound 144 was synthesized following the similar route of Example 26, using 5-fluoro-2-methylpyridine in step C.

¹H NMR (400 MHz, DMSO-d₆) δ 8.70 (s, 1H), 8.52 (d, J=2.8 Hz, 1H), 7.75-7.80 (m, 1H), 7.70 (s, 1H), 7.59-7.62 (m, 1H), 7.44 (s, 1H), 7.12 (d, J=8.0 Hz, 1H), 7.01-7.08 (m, 1H), 6.89-6.93 (m, 3H), 6.81-6.85 (m, 2H), 5.17 (s, 2H), 3.23 (d, J=13.6 Hz, 1H), 3.05 (d, J=13.6 Hz, 1H), 2.74-2.80 (m, 2H), 2.35-2.40 (m, 1H), 1.55 (s, 3H), 1.02 (d, J=6.8 Hz, 3H), 0.92 (d, J=6.8 Hz, 3H). ¹⁹F NMR (376 MHz, DMSO-d₆) δ −116.70, −128.73. LC-MS: m/z 598.0 (M+H)⁺.

### (S)-7-{3-[(5-chloro-2-pyridyl)methyl]-2-oxo-1,3-benzoxazol-6-yl}-3-[(p-fluorophenyl)methyl]-5-isobutyl-3-methyl-1-oxo-2,4-diaza-6-indancarboxamide (Compound 145)

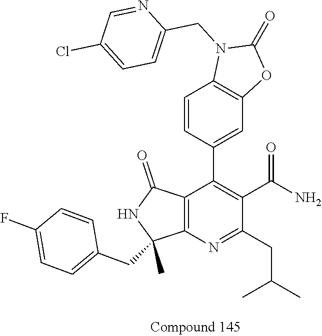 [SMILES: CC(C)Cc1nc2c(c(-c3ccc4c(c3)oc(=O)n4Cc3ccc(Cl)cn3)c1C(N)=O)C(=O)N[C@@]2(C)Cc1ccc(F)cc1]

Compound 145 was synthesized following the similar route of Example 26, using 5-chloro-2-(chloromethyl)pyridine, hydrochloride in step D.

¹H NMR (400 MHz, DMSO-d₆) δ 8.71 (s, 1H), 8.57 (d, J=2.4 Hz, 1H), 7.97 (dd, J=8.4, 2.4 Hz, 1H), 7.70 (s, 1H), 7.56 ((d, J=8.4 Hz, 1H), 7.44 (s, 1H), 7.12 (d, J=8.0 Hz, 1H), 7.05 (s, 1H), 6.89-6.96 (m, 3H), 6.81-6.85 (m, 2H), 5.18 (s, 2H), 3.23 (d, J=13.2 Hz, 1H), 3.05 (d, J=13.2 Hz, 1H), 2.71-2.80 (m, 2H), 2.32-2.42 (m, 1H), 1.55 (s, 3H), 1.02 (d, J=6.8 Hz, 3H), 0.92 (d, J=6.8 Hz, 3H). ¹⁹F NMR (376 MHz, DMSO-d₆) δ −116.69. LC-MS: m/z 614.3 (M+H)⁺.

### (S)-7-{3-[(3,5-difluoro-2-pyridyl)methyl]-2-oxo-1,3-benzoxazol-6-yl}-3-[(p-fluorophenyl)methyl]-5-isobutyl-3-methyl-1-oxo-2,4-diaza-6-indancarboxamide (Compound 146)

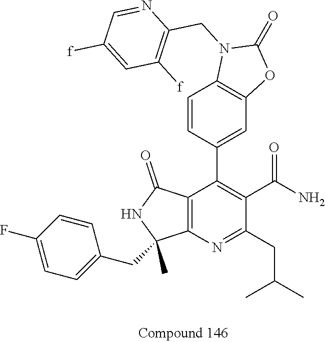

Compound 146 was synthesized following the similar route of Example 26, using 2-(chloromethyl)-3,5-difluoropyridine in step D.

¹H NMR (400 MHz, DMSO-d₆) δ 8.71 (s, 1H), 8.47 (d, J=2.4 Hz, 1H), 8.01-8.06 (m, 1H), 7.71 (s, 1H), 7.45 (s, 1H), 7.16 (d, J=8.0 Hz, 1H), 7.06 (s, 1H), 6.89-6.96 (m, 3H), 6.82-6.85 (m, 2H), 5.26 (s, 2H), 3.25 (d, J=13.2 Hz, 1H), 3.07 (d, J=13.2 Hz, 1H), 2.74-2.79 (m, 2H), 2.37-2.49 (m, 1H), 1.55 (s, 3H), 1.02 (d, J=6.8 Hz, 3H), 0.93 (d, J=6.8 Hz, 3H). ¹⁹F NMR (376 MHz, DMSO-d₆) δ −116.70, −121.63, −124.55. LC-MS: m/z 616.6 (M+H)⁺.

### Example 27

### (S)-7-{3-[1-(5-cyano-2-pyridyl) propyl]-2-oxo-1,3-benzoxazol-6-yl}-3-[(p-fluorophenyl)methyl]-5-isobutyl-3-methyl-1-oxo-2,4-diaza-6-indancarboxamide (Compound 147)

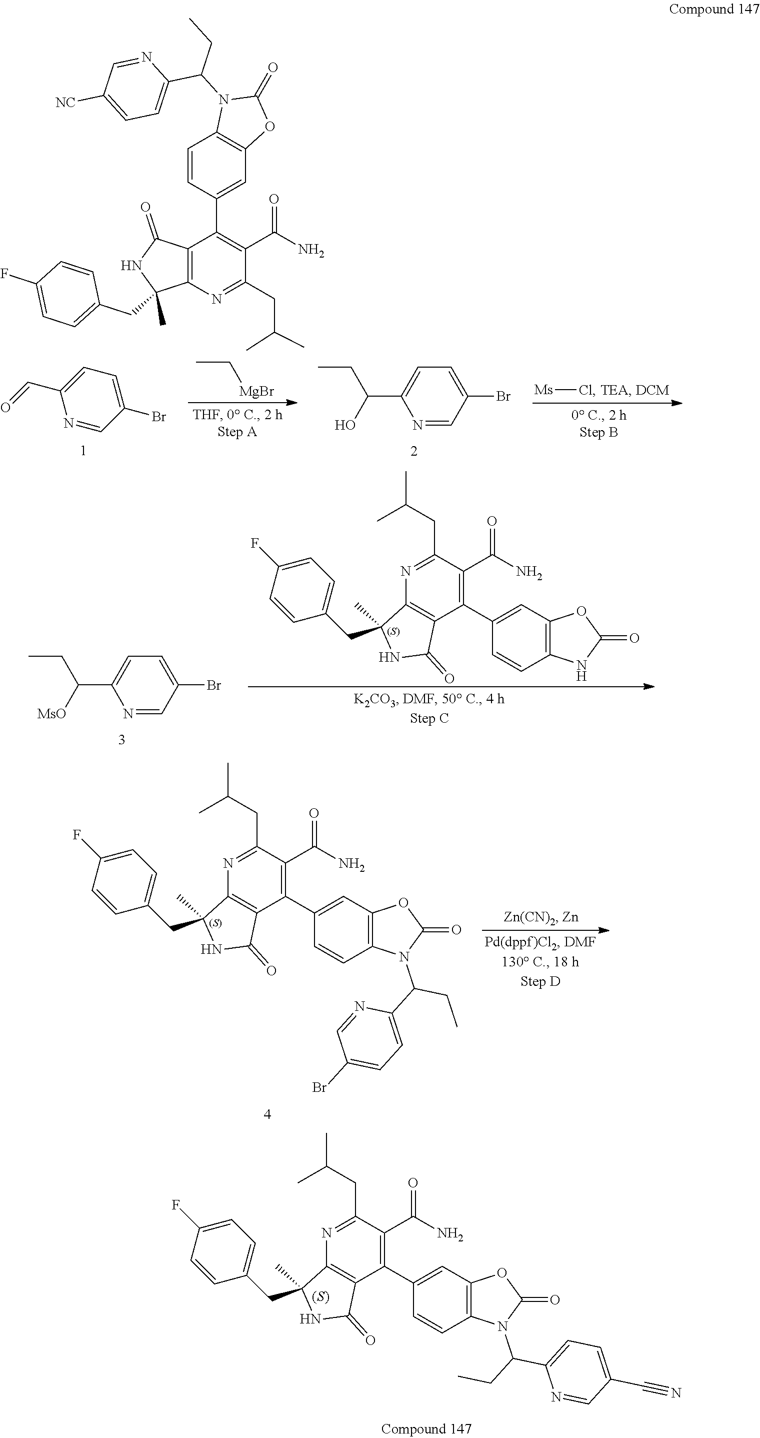

### Step A: 1-(5-bromopyridin-2-yl) propan-1-ol

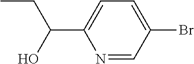

To a solution of 5-bromopicolinaldehyde (1 g, 5.376 mmol) in tetrahydrofuran (20 mL) was added ethyl magnesium bromide (4.8 mL, 13.44 mmol) at 0° C. under N₂. The reaction mixture was stirred at 0° C. for 2 hrs. The reaction mixture was quenched by aq. NH₄Cl (10 mL), extracted with EtOAc (30 mL), the organic layer was washed with brine (30 mL), dried over anhydrous Na₂SO₄, filtered and concentrated under vacuum. The resulting residue was purified by silica gel chromatography (PE/EtOAc=3/1) to afford 1-(5-bromopyridin-2-yl) propan-1-ol (600 mg, 55.61%). ¹H NMR (400 MHz, CDCl₃) δ 8.60 (d, J=2.0 Hz, 1H), 7.81 (dd, J=8.4, 2.0 Hz, 1H), 7.20 (d, J=8.4 Hz, 1H), 4.65-4.68 (m, 1H), 1.83-1.89 (m, 1H), 1.67-1.75 (m, 1H), 0.94 (t, J=7.2 Hz, 3H).

### Step B: 1-(5-bromopyridin-2-yl) propyl methanesulfonate

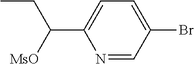

To a solution of 1-(5-bromopyridin-2-yl) propan-1-ol (600 mg, 2.777 mmol) in DCM (10 mL) were added triethylamine (1.930 mL, 13.884 mmol) and methanesulfonyl chloride (0.430 mL, 5.553 mmol) at 0° C. The mixture was stirred at 0° C. for 2 hours. The reaction mixture was quenched by water (10 mL) and extracted with DCM (30 mL), the combined organic layer was washed with brine, dried over anhydrous Na₂SO₄, filtered and the residue was concentrated in vacuo to afford 1-(5-bromopyridin-2-yl) propyl methanesulfonate (642 mg, 78.60%) which was used directly in the next step without further purification. ¹H NMR (400 MHz, CDCl₃) δ 8.66 (d, J=2.0 Hz, 1H), 7.88 (dd, J=8.4, 2.0 Hz, 1H), 7.36 (d, J=8.4 Hz, 1H), 5.53 (t, J=6.4 Hz, 1H), 2.94 (s, 3H), 2.02-2.10 (m, 2H), 0.99 (t, J=7.2 Hz, 3H).

### Step C: (7S)-4-(3-(1-(5-bromopyridin-2-yl) propyl)-2-oxo-2,3-dihydrobenzo[d]oxazol-6-yl)-7-(4-fluorobenzyl)-2-isobutyl-7-methyl-5-oxo-6,7-dihydro-5H-pyrrolo[3,4-b]pyridine-3-carboxamide

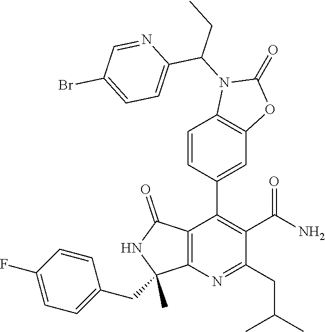 [SMILES: CCC(c1ccc(Br)cn1)n1c(=O)oc2cc(-c3c(C(N)=O)c(CC(C)C)nc4c3C(=O)N[C@@]4(C)Cc3ccc(F)cc3)ccc21]

To a solution of (S)-7-(4-fluorobenzyl)-2-isobutyl-7-methyl-5-oxo-4-(2-oxo-2,3-dihydrobenzo[d]oxazol-6-yl)-6,7-dihydro-5H-pyrrolo[3,4-b]pyridine-3-carboxamide (41.52 mg, 0.085 mmol) in DMF (5 mL) were added 1-(5-bromopyridin-2-yl) propyl methanesulfonate (50 mg, 0.170 mmol) and potassium carbonate (58.73 mg, 0.425 mmol) at 25° C. under N₂. The reaction mixture was stirred at 50° C. for 6 hours. The reaction mixture was quenched by NH₄Cl (10 mL), extracted with EtOAc (30 mL×2), the combined organic layers were washed with brine (30 mL×3), dried over anhydrous Na₂SO₄, filtered and concentrated under vacuum. The resulting residue was purified by prep-TLC (DCM/MeOH=10/1) to afford (7S)-4-(3-(1-(5-bromopyridin-2-yl) propyl)-2-oxo-2,3-dihydrobenzo[d]oxazol-6-yl)-7-(4-fluorobenzyl)-2-isobutyl-7-methyl-5-oxo-6,7-dihydro-5H-pyrrolo[3,4-b]pyridine-3-carboxamide (42 mg, 71.97%). LC-MS: m/z 688.1 (M+H)⁺.

### Step D: (S)-7-{3-[1-(5-cyano-2-pyridyl) propyl]-2-oxo-1,3-benzoxazol-6-yl}-3-[(p-fluorophenyl)methyl]-5-isobutyl-3-methyl-1-oxo-2,4-diaza-6-indancarboxamide

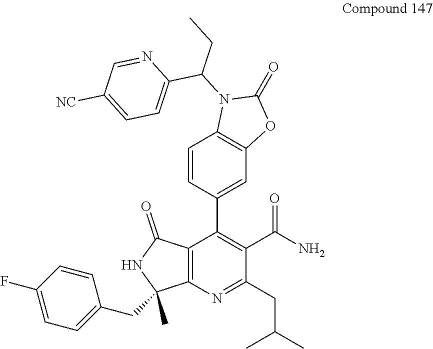 [SMILES: CCC(c1ccc(C#N)cn1)n1c(=O)oc2cc(-c3c(C(N)=O)c(CC(C)C)nc4c3C(=O)N[C@@]4(C)Cc3ccc(F)cc3)ccc21]

To a solution of (7S)-4-(3-(1-(5-bromopyridin-2-yl) propyl)-2-oxo-2,3-dihydrobenzo[d]oxazol-6-yl)-7-(4-fluorobenzyl)-2-isobutyl-7-methyl-5-oxo-6,7-dihydro-5H-pyrrolo[3,4-b]pyridine-3-carboxamide (42 mg, 0.061 mmol) in DMA (3 mL) were added Pd(dppf)Cl₂ (4.44 mg, 0.006 mmol) 2, Zn(CN)₂ (21.55 mg, 0.184 mmol) and TMEDA (3.55 mg, 0.031 mmol). The reaction mixture was stirred at 200° C. for 30 mins under microwave. After the reaction was completed, water (10 mL) was added, the mixture was extracted with EtOAc (30 mL), the combined organic layers were washed with brine (15 mL), dried over anhydrous Na₂SO₄, filtered and concentrated under vacuum. The resulting residue was purified by silica gel chromatography (DCM/MeOH=10/1) to afford a crude product (20 mg), which was then purified by prep-HPLC (Waters 3767/Qda Column: SunFire Sunfire C18, 19*250 mm*10 μm; Mobile Phase A: 0.1% FA/H2O, B: ACN; Flow rate: 40 mL/min; Gradient: 46%; Retention Time: 9.2-10.1 min of 17 min) to afford (S)-7-{3-[1-(5-cyano-2-pyridyl) propyl]-2-oxo-1,3-benzoxazol-6-yl}-3-[(p-fluorophenyl)methyl]-5-isobutyl-3-methyl-1-oxo-2,4-diaza-6-indancarboxamide (2.84 mg, 7.03%).

¹H NMR (400 MHz, DMSO-d₆) δ 9.00 (s, 1H), 8.72 (s, 1H), 8.36 (dd, J=8.4, 2.4 Hz, 1H), 7.78 (d, J=8.4 Hz, 1H), 7.70 (s, 1H), 7.48 (s, 1H), 7.20 (dd, J=8.0, 3.2 Hz, 1H), 7.04-7.06 (m, 1H), 6.86-6.94 (m, 3H), 6.80-6.84 (m, 2H), 5.54 (t, J=8.0 Hz, 1H), 3.23 (d, J=13.6 Hz, 1H), 3.04 (d, J=13.6 Hz, 1H), 2.74-2.78 (m, 2H), 2.35-2.47 (m, 3H), 1.55 (s, 3H), 1.01 (d, J=6.4 Hz, 3H), 0.91-0.96 (m, 6H). ¹⁹F NMR (376 MHz, DMSO-d₆) δ −116.68. LC-MS: m/z 633.4 (M+H)⁺.

### (S)-7-{3-[1-(4-cyano-2-fluorophenyl)ethyl]-2-oxo-1,3-benzoxazol-6-yl}-3-[(p-fluorophenyl)methyl]-5-isobutyl-3-methyl-1-oxo-2,4-diaza-6-indancarboxamide (Compound 148)

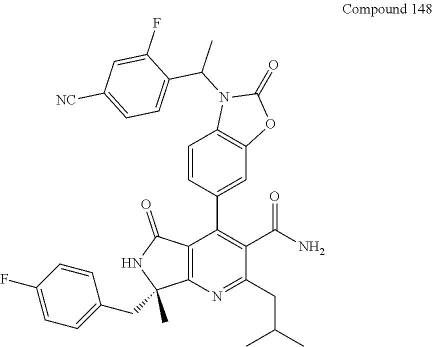 [SMILES: CC(C)Cc1nc2c(c(-c3ccc4c(c3)oc(=O)n4C(C)c3ccc(C#N)cc3F)c1C(N)=O)C(=O)N[C@@]2(C)Cc1ccc(F)cc1]

Compound 148 was synthesized following the similar route of Example 27, using 1-(4-cyano-2-fluorophenyl)ethyl methanesulfonate in step C. ¹H NMR (400 MHz, DMSO-d₆) δ 8.71 (s, 1H), 7.95 (t, J=7.2 Hz, 1H), 7.86 (d, J=10.4 Hz, 1H), 7.80 (d, J=8.0 Hz, 1H), 7.70 (s, 1H), 7.46 (s, 1H), 7.14 (dd, J=8.4, 4.0 Hz, 1H), 7.03 (s, 1H), 6.86-6.94 (m, 3H), 6.80-6.84 (m, 2H), 5.83-5.88 (m, 1H), 3.23 (d, J=13.6 Hz, 1H), 3.05 (d, J=13.6 Hz, 1H), 2.70-2.81 (m, 2H), 2.32-2.40 (m, 1H), 1.88 (d, J=7.2 Hz, 3H), 1.54 (s, 3H), 1.02 (d, J=6.8 Hz, 3H), 0.91 (d, J=6.8 Hz, 3H). ¹⁹F NMR (376 MHz, DMSO-d₆) δ −113.08, −116.67. LC-MS: m/z 636.1 (M+H)⁺.

### (S)-7-{3-[1-(4-cyano-3-fluorophenyl)ethyl]-2-oxo-1,3-benzoxazol-6-yl}-3-[(p-fluorophenyl)methyl]-5-isobutyl-3-methyl-1-oxo-2,4-diaza-6-indancarboxamide (Compound 149)

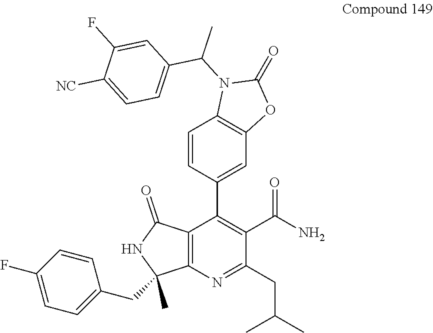 [SMILES: CC(C)Cc1nc2c(c(-c3ccc4c(c3)oc(=O)n4C(C)c3ccc(C#N)c(F)c3)c1C(N)=O)C(=O)N[C@@]2(C)Cc1ccc(F)cc1]

Compound 149 was synthesized following the similar route of Example 27, using 1-(4-cyano-3-fluorophenyl)ethyl methanesulfonate in step C. ¹H NMR (400 MHz, CD₃OD) δ 7.75 (t, J=7.4 Hz, 1H), 7.50 (d, J=10.4 Hz, 1H), 7.45 (d, J=8.0 Hz, 1H), 7.04-7.11 (m, 1H), 6.95-6.97 (m, 2H), 6.84-6.88 (m, 2H), 6.80 (t, J=8.4 Hz, 2H), 5.67-5.72 (m, 1H), 3.39 (d, J=13.6 Hz, 1H), 3.15 (d, J=13.6 Hz, 1H), 2.87-2.90 (m, 2H), 2.41-2.48 (m, 1H), 1.93 (d, J=6.8 Hz, 3H), 1.66 (s, 3H), 1.07 (d, J=6.4 Hz, 3H), 1.01 (d, J=6.4 Hz, 3H). ¹⁹F NMR (376 MHz, CD₃OD) δ −108.73, −118.37. LC-MS: m/z 636.5 (M+H)⁺.

### (S)-7-{3-[(R)-1-(4-cyano-3-fluorophenyl)ethyl]-2-oxo-1,3-benzoxazol-6-yl}-3-[(p-fluorophenyl)methyl]-5-isobutyl-3-methyl-1-oxo-2,4-diaza-6-indancarboxamide and(S)-7-{3-[(S)-1-(4-cyano-3-fluorophenyl)ethyl]-2-oxo-1,3-benzoxazol-6-yl}-3-[(p-fluorophenyl)methyl]-5-isobutyl-3-methyl-1-oxo-2,4-diaza-6-indancarboxamide (Compound 150 and Compound 151)

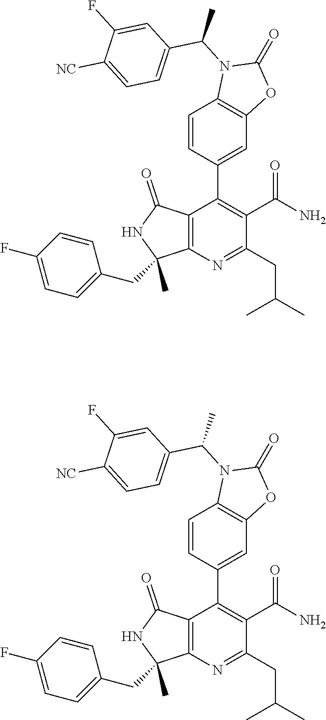 [SMILES: CC(C)Cc1nc2c(c(-c3ccc4c(c3)oc(=O)n4[C@H](C)c3ccc(C#N)c(F)c3)c1C(N)=O)C(=O)N[C@@]2(C)Cc1ccc(F)cc1 ; CC(C)Cc1nc2c(c(-c3ccc4c(c3)oc(=O)n4[C@@H](C)c3ccc(C#N)c(F)c3)c1C(N)=O)C(=O)N[C@@]2(C)Cc1ccc(F)cc1]

(S)-7-{3-[1-(4-cyano-3-fluorophenyl)ethyl]-2-oxo-1,3-benzoxazol-6-yl}-3-[(p-fluorophenyl)methyl]-5-isobutyl-3-methyl-1-oxo-2,4-diaza-6-indancarboxamide (32 mg, 0.050 mmol) was separated by SFC (Column: CHIRALCEL OZ-H (OZHOCD-VF004); Column size: 0.46 cm I.D.*15 cm L, 10 μm; Mobile Phase A: Hexane; Mobile Phase B: EtOH; Gradient: B=50%). Flow rate: 1.0 mL/min, Column temp.: 35° C.) to afford (S)-7-{3-[(R)-1-(4-cyano-3-fluorophenyl)ethyl]-2-oxo-1,3-benzoxazol-6-yl}-3-[(p-fluorophenyl)methyl]-5-isobutyl-3-methyl-1-oxo-2,4-diaza-6-indancarboxamide and (S)-7-{3-[(S)-1-(4-cyano-3-fluorophenyl)ethyl]-2-oxo-1,3-benzoxazol-6-yl}-3-[(p-fluorophenyl)methyl]-5-isobutyl-3-methyl-1-oxo-2,4-diaza-6-indancarboxamide.

Compound 150, enantiomer 1 (8.56 mg, 26.8%): ¹H NMR (400 MHz, DMSO-d₆) δ 8.69 (s, 1H), 7.94 (t, J=7.6 Hz, 1H), 7.69-7.71 (m, 2H), 7.49 (dd, J=8.0, 1.2 Hz, 1H), 7.44 (s, 1H), 7.17 (d, J=8.0 Hz, 1H), 7.03-7.07 (m, 1H), 6.88-6.92 (m, 3H), 6.80-6.84 (m, 2H), 5.66-5.72 (m, 1H), 3.24 (d, J=13.2 Hz, 1H), 3.06 (d, J=13.2 Hz, 1H), 2.71-2.81 (m, 2H), 2.35-2.40 (m, 1H), 1.89 (d, J=7.2 Hz, 3H), 1.55 (s, 3H), 1.02 (d, J=6.8 Hz, 3H), 0.92 (d, J=6.8 Hz, 3H). ¹⁹F NMR (376 MHz, DMSO-d₆) δ −107.57, −116.68. LC-MS: m/z 636.3 (M+H)⁺.

Compound 151, enantiomer 2 (7.04 mg, 22.0%): ¹H NMR (400 MHz, DMSO-d₆) δ 8.69 (s, 1H), 7.94 (t, J=7.2 Hz, 1H), 7.69-7.71 (m, 2H), 7.48 (dd, J=8.0, 1.2 Hz, 1H), 7.44 (s, 1H), 7.16 (d, J=8.4 Hz, 1H), 7.03-7.06 (m, 1H), 6.88-6.92 (m, 3H), 6.80-6.84 (m, 2H), 5.66-5.72 (m, 1H), 3.24 (d, J=13.6 Hz, 1H), 3.06 (d, J=13.6 Hz, 1H), 2.74-2.78 (m, 2H), 2.35-2.38 (m, 1H), 1.89 (d, J=7.2 Hz, 3H), 1.55 (s, 3H), 1.02 (d, J=6.8 Hz, 3H), 0.92 (d, J=6.8 Hz, 3H). ¹⁹F NMR (376 MHz, DMSO-d₆) δ −107.59, −116.68. LC-MS: m/z 636.4 (M+H)⁺.

### Example 28

### (S)-7-{7-[(R)-4-aza-1-indanylamino]-1-thia-6-aza-2-indenyl}-3-[(p-fluorophenyl)methyl]-6-(hydroxymethyl)-5-isobutyl-3-methyl-2,4-diaza-1-indanone (Compound 152) and(S)-7-{7-[(R)-4-aza-1-indanylamino]-1-thia-6-aza-2-indenyl}-3-[(p-fluorophenyl)methyl]-5-isobutyl-3-methyl-1-oxo-2,4-diaza-6-indancarbaldehyde (Compound 153)

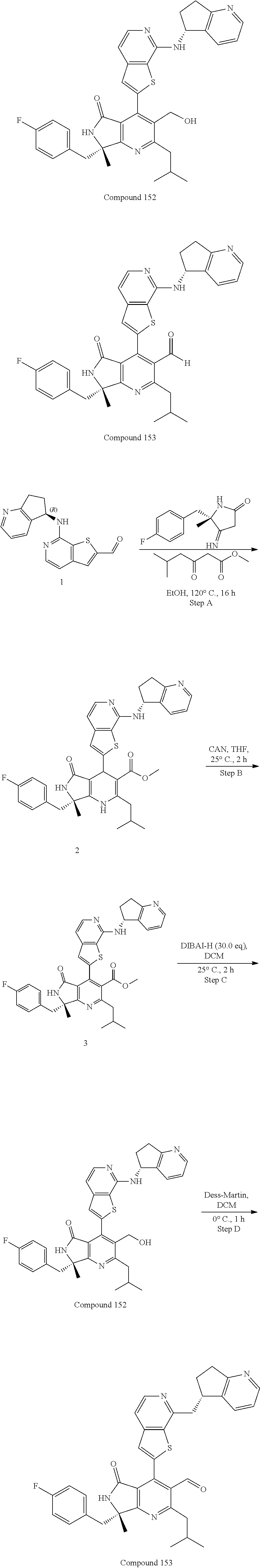

### Step A: methyl(7S)-7-[(4-fluorophenyl)methyl]-4-(7-{[(5R)-6,7-dihydro-5H-cyclopenta[1,2-b]pyridin-5-yl]amino}thieno[2,3-c]pyridin-2-yl)-7-methyl-2-(2-methylpropyl)-5-oxo-4,5,6,7-tetrahydro-1H-pyrrolo[4,3-b]pyridine-3-carboxylate

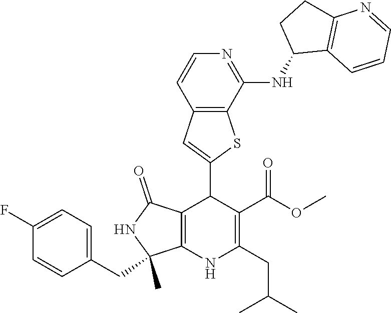 [SMILES: COC(=O)C1=C(CC(C)C)NC2=C(C(=O)N[C@@]2(C)Cc2ccc(F)cc2)C1c1cc2ccnc(N[C@@H]3CCc4ncccc43)c2s1]

To a solution of (5S)-4-azanylidene-5-[(4-fluorophenyl)methyl]-5-methyltetrahydropyrrol-2-one (100 mg, 0.454 mmol) in EtOH (5 mL) were added 7-{[(5R)-6,7-dihydro-5H-cyclopenta[1,2-b]pyridin-5-yl]amino}thieno[2,3-c]pyridine-2-carbaldehyde which was synthesized following the similar route of Example 7, using 7-chlorothieno[2,3-c]pyridine in step A (134.10 mg, 0.454 mmol) and methyl 5-methyl-3-oxohexanoate (143.65 mg, 0.908 mmol), the reaction mixture was stirred at 120° C. for 16 hours under N₂ in a sealed tube. The reaction mixture was concentrated under vacuum to afford methyl(7S)-7-[(4-fluorophenyl)methyl]-4-(7-{[(5R)-6,7-dihydro-5H-cyclopenta[1,2-b]pyridin-5-yl]amino}thieno[2,3-c]pyridin-2-yl)-7-methyl-2-(2-methylpropyl)-5-oxo-4,5,6,7-tetrahydro-1H-pyrrolo[4,3-b]pyridine-3-carboxylate (289 mg, 99.80%) which was used in the next step without further purification. LC-MS: m/z 637.2 (M+H)⁺.

### Step B: methyl(S)-7-{7-[(R)-4-aza-1-indanylamino]-1-thia-6-aza-2-indenyl}-3-[(p-fluorophenyl)methyl]-5-isobutyl-3-methyl-1-oxo-2,4-diaza-6-indancarboxylate

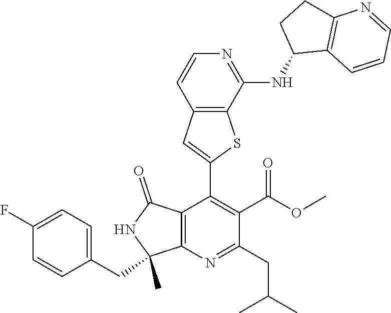 [SMILES: COC(=O)c1c(CC(C)C)nc2c(c1-c1cc3ccnc(N[C@@H]4CCc5ncccc54)c3s1)C(=O)N[C@@]2(C)Cc1ccc(F)cc1]

To a solution of methyl(7S)-7-[(4-fluorophenyl)methyl]-4-(7-{[(5R)-6,7-dihydro-5H-cyclopenta[1,2-b]pyridin-5-yl]amino}thieno[2,3-c]pyridin-2-yl)-7-methyl-2-(2-methylpropyl)-5-oxo-4,5,6,7-tetrahydro-1H-pyrrolo[4,3-b]pyridine-3-carboxylate (289 mg, 0.453 mmol) in THF (5 mL) was added Ceric ammonium nitrate (496.84 mg, 0.906 mmol). The reaction mixture was stirred at 25° C. for 2 hours under N₂. To the reaction mixture was added aq. NaHCO₃ (30 mL). The resulting residue was extracted with EA (30 mL×2), the combined organic layers were washed with brine (30 mL×3), dried over anhydrous Na₂SO₄, filtered and concentrated under vacuum. The resulting residue was purified by silica gel chromatography (eluting with DCM/MeOH=10/1) to afford methyl(S)-7-{7-[(R)-4-aza-1-indanylamino]-1-thia-6-aza-2-indenyl}-3-[(p-fluorophenyl)methyl]-5-isobutyl-3-methyl-1-oxo-2,4-diaza-6-indancarboxylate (136.0 mg, 47.2%). LC-MS: m/z 636.1 (M+H)⁺.

### Step C: (S)-7-{7-[(R)-4-aza-1-indanylamino]-1-thia-6-aza-2-indenyl}-3-[(p-fluorophenyl)methyl]-6-(hydroxymethyl)-5-isobutyl-3-methyl-2,4-diaza-1-indanone

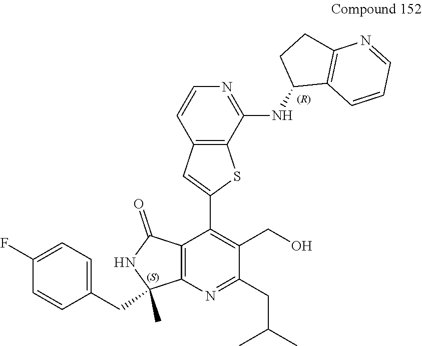

A solution of methyl(S)-7-{7-[(R)-4-aza-1-indanylamino]-1-thia-6-aza-2-indenyl}-3-[(p-fluorophenyl)methyl]-5-isobutyl-3-methyl-1-oxo-2,4-diaza-6-indancarboxylate (45 mg, 0.071 mmol) in DCM (15 mL) was added DIBAL-H (1.42 mL, 2.131 mmol) at 0° C., the reaction mixture was stirred at 0° C. for 1 hour under N₂. The reaction mixture was quenched by citric acid (30% in water, 3 mL), the resulting mixture was extracted with DCM (20 mL×2), washed with brine (20 mL), dried over anhydrous Na₂SO₄, filtered and concentrated under vacuum. The resulting residue was purified by prop-TLC (DCM/CH₃OH=10/1) and prep-HPLC (Waters 3767/Qda Column: SunFire Sunfire C18, 19*250 mm*10 μm; Mobile Phase A: 10 mmol NH₄HCO₃/H2O, B: ACN; Flow rate: 20 mL/min; Gradient: 62%-72%; Retention Time: 9.3-10 min of 16 min) to afford (S)-7-{7-[(R)-4-aza-1-indanylamino]-1-thia-6-aza-2-indenyl}-3-[(p-fluorophenyl)methyl]-6-(hydroxymethyl)-5-isobutyl-3-methyl-2,4-diaza-1-indanone (6.1 mg, 14.15%).

¹H NMR (400 MHz, DMSO-d₆) δ 8.47-8.66 (m, 2H), 8.34 (s, 1H), 7.94 (d, J=5.6 Hz, 1H), 7.73 (d, J=7.2 Hz, 1H), 7.18-7.21 (m, 1H), 7.12 (s, 1H), 7.10 (d, J=5.6 Hz, 1H), 6.84-6.88 (m, 2H), 6.75-6.79 (m, 2H), 5.86-5.90 (t, J=9.2 Hz, 1H), 4.54-4.57 (m, 2H), 4.49 (d, J=12.0 Hz, 1H), 3.40 (d, J=14.0 Hz, 1H), 3.12-3.16 (m, 2H), 3.00-3.07 (m, 2H), 2.68-2.75 (m, 1H), 2.42-2.48 (m, 1H), 2.15 (s, 1H), 2.07-2.12 (m, 1H), 1.66 (s, 3H), 1.12 (d, J=6.8 Hz, 3H), 1.04 (d, J=6.8 Hz, 3H). ¹⁹F NMR (376 MHz, DMSO-d₆) δ −118.35. LC-MS: m/z 608.3 (M+H)⁺.

### Step D: (S)-7-{7-[(R)-4-aza-1-indanylamino]-1-thia-6-aza-2-indenyl}-3-[(p-fluorophenyl)methyl]-5-isobutyl-3-methyl-1-oxo-2,4-diaza-6-indancarbaldehyde

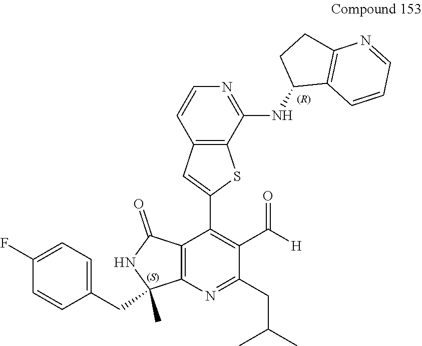

To a solution of(S)-7-{7-[(R)-4-aza-1-indanylamino]-1-thia-6-aza-2-indenyl}-3-[(p-fluorophenyl)methyl]-6-(hydroxymethyl)-5-isobutyl-3-methyl-2,4-diaza-1-indanone (50 mg, 0.082 mmol) in DCM (20 mL) was added Dess-Martin periodinane (69.79 mg, 0.165 mmol), the reaction mixture was stirred at 0° C. for 1 hour under N₂. The reaction mixture was filtered and the filtrate was concentrated under vacuum. The resulting residue was purified by prep-TLC (DCM/CH₃OH=10/1) and prep-HPLC (Waters 3767/Qda Column: SunFire Sunfire C18, 19*250 mm*10 μm; Mobile Phase A: 0.1% FA/H2O, B: ACN; Flow rate: 20 mL/min; Gradient: 77%-82%; Retention Time: 9.3-11 min of 16 min) to afford (S)-7-{7-[(R)-4-aza-1-indanylamino]-1-thia-6-aza-2-indenyl}-3-[(p-fluorophenyl)methyl]-5-isobutyl-3-methyl-1-oxo-2,4-diaza-6-indancarbaldehyde (2.03 mg, 4.06%).

¹H NMR (400 MHz, DMSO-d₆) δ 9.93 (s, 1H), 9.00 (s, 1H), 8.37 (d, J=4.8 Hz, 1H), 8.00 (d, J=5.2 Hz, 1H), 7.61 (d, J=7.2 Hz, 1H), 7.33 (d, J=8.0 Hz, 1H), 7.28 (s, 1H), 7.10-7.14 (m, 1H), 7.09 (d, J=6 Hz, 1H), 6.84-6.93 (m, 4H), 5.84-5.91 (m, 1H), 3.25-3.29 (m, 1H), 3.11-3.17 (m, 2H), 2.99-3.04 (m, 2H), 2.88-2.97 (m, 2H), 2.17-2.25 (m, 1H), 2.02-2.10 (m, 1H), 1.59 (s, 3H), 1.03 (d, J=6.8 Hz, 3H), 0.93 (d, J=6.8 Hz, 3H). ¹⁹F NMR (376 MHz, DMSO-d₆) δ −116.19. LC-MS: m/z 606.2 (M+H)⁺.

### Example 29

### N-[(R)-1-indanyl]-5-{3-carbamoyl-7-[(p-fluorophenyl)methyl]-2-isobutyl-4-quinolyl}-2-thenamide (Compound 160)

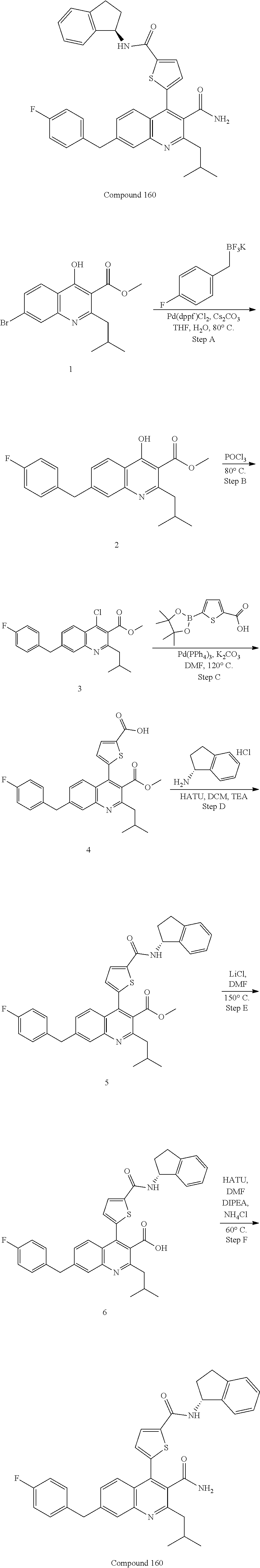

### Step A: methyl 7-[(4-fluorophenyl)methyl]-4-hydroxy-2-isobutyl-quinoline-3-carboxylate

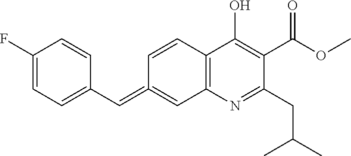

A mixture of methyl 7-bromo-4-hydroxy-2-isobutyl-quinoline-3-carboxylate (2 g, 5.91 mmol), trifluoro(4-fluorobenzyl)-14-borane, potassium salt (1.41 g, 6.51 mmol), cyclopentyl(diphenyl)phosphane;dichloropalladium;iron (216.36 mg, 295.69 μmol) and Cs₂CO₃ (3.85 g, 11.83 mmol) in the mixture of THF (20 mL) and H₂O (2 mL) was degassed and purged with N₂ for 3 times, and then the mixture was stirred at 80° C. for 5 hours under N₂ atmosphere. The reaction mixture was concentrated and the residue was purified by column chromatography (SiO₂, DCM/MeOH=1/0 to 10/1) to afford methyl 7-[(4-fluorophenyl)methyl]-4-hydroxy-2-isobutyl-quinoline-3-carboxylate (2.4 g, 88.37%, 80% purity).

¹H NMR (400 MHz, DMSO-d₆) δ 11.61 (s, 1H), 7.96 (d, J=8.0 Hz, 1H), 7.32 (dd, J=8.0, 5.6 Hz, 2H), 7.21-7.27 (m, 2H), 7.13-7.19 (m, 2H), 4.08 (s, 2H), 3.73 (s, 3H), 2.41-2.46 (m, 2H), 1.92-2.06 (m, 1H), 0.88 (d, J=6.4 Hz, 6H).

### Step B: methyl 4-chloro-7-[(4-fluorophenyl)methyl]-2-isobutyl-quinoline-3-carboxylate

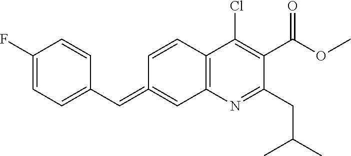

A mixture of methyl 7-[(4-fluorophenyl)methyl]-4-hydroxy-2-isobutyl-quinoline-3-carboxylate (2.2 g, 5.99 mmol) in POCl₃ (25 mL) was degassed and purged with N₂ for 3 times, and then the mixture was stirred at 80° C. for 16 hours under N₂ atmosphere. The solution was concentrated and to the residue was added water (50 mL), and NaHCO₃ (20 mL). The mixture was extracted with EtOAc (30 mL×3). The combined organic layers were washed with brine (30 mL), dried over anhydrous Na₂SO₄, filtered and concentrated under reduced pressure to afford a residue. The residue was purified by column chromatography (SiO₂, PE/EtOAc=1/0 to 10/1) to afford methyl 4-chloro-7-[(4-fluorophenyl)methyl]-2-isobutyl-quinoline-3-carboxylate (1.61 g, 69.68%).

¹H NMR (400 MHz, CDCl₃) δ 8.13 (d, J=8.4 Hz, 1H), 7.84 (s, 1H), 7.44 (d, J=8.4 Hz, 1H), 7.16-7.23 (m, 2H), 7.00 (t, J=8.4 Hz, 2H), 4.16 (s, 2H), 4.02 (s, 3H), 2.80 (d, J=7.2 Hz, 2H), 2.24-2.32 (m, 1H), 0.96 (d, J=6.8 Hz, 6H).

### Step C: 5-[7-[(4-fluorophenyl)methyl]-2-isobutyl-3-methoxycarbonyl-4-quinolyl]thiophene-2-carboxylic acid

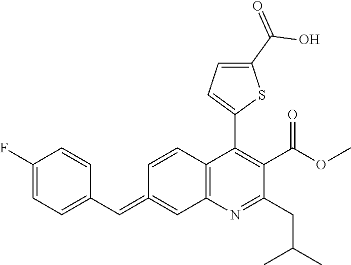

A mixture of methyl 4-chloro-7-[(4-fluorophenyl)methyl]-2-isobutyl-quinoline-3-carboxylate (1 g, 2.59 mmol), 5-(4,4,5,5-tetramethyl-1,3,2-dioxaborolan-2-yl)thiophene-2-carboxylic acid (724.41 mg, 2.85 mmol), K₂CO₃ (1.07 g, 7.77 mmol) and palladium; triphenylphosphane (95.55 mg, 259.16 μmol) in DMF (10 mL) was degassed and purged with N₂ for 3 times, and then the mixture was stirred at 120° C. for 16 hours under N₂ atmosphere. The reaction mixture was diluted with H₂O (100 mL) and extracted with EtOAc (20 mL×3). The combined organic layers were washed with H₂O (50 mL×2), dried over Na₂SO₄, filtered and concentrated under reduced pressure to afford a residue. The residue was purified by column chromatography (SiO₂, PE/EtOAc=1/0 to 3/1) to afford 5-[7-[(4-fluorophenyl)methyl]-2-isobutyl-3-methoxycarbonyl-4-quinolyl]thiophene-2-carboxylic acid (480 mg, crude) which was used in the next step without further purification.

### Step D: methyl 7-[(4-fluorophenyl)methyl]-4-[5-[[(1R)-indan-1-yl]carbamoyl]-2-thienyl]-2-isobutyl-quinoline-3-carboxylate

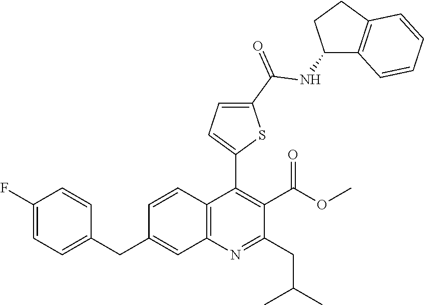 [SMILES: COC(=O)c1c(CC(C)C)nc2cc(Cc3ccc(F)cc3)ccc2c1-c1ccc(C(=O)N[C@@H]2CCc3ccccc32)s1]

To a solution of 5-[7-[(4-fluorophenyl)methyl]-2-isobutyl-3-methoxycarbonyl-4-quinolyl]thiophene-2-carboxylic acid (380 mg, 795.73 μmol) in DCM (10 mL) was added TEA (322.08 mg, 3.18 mmol) and HATU (453.84 mg, 1.19 mmol). The mixture was stirred at 25° C. for 30 min. Then (1R)-indan-1-amine;hydrochloride (162.00 mg, 954.88 μmol) was added to the solution and the mixture was stirred at 25° C. for 2.5 hours. The solution was concentrated and the residue was purified by column chromatography (SiO₂, PE/EtOAc=1/0 to 5/1) to afford methyl 7-[(4-fluorophenyl)methyl]-4-[5-[[(1R)-indan-1-yl]carbamoyl]-2-thienyl]-2-isobutyl-quinoline-3-carboxylate (353 mg, 74.84%).

¹H NMR (400 MHz, CDCl₃) δ 7.89 (s, 1H), 7.73 (d, J=8.4 Hz, 1H), 7.53 (d, J=3.6 Hz, 1H), 7.38-7.46 (m, 2H), 7.29-7.34 (m, 2H), 7.20 (dd, J=8.0, 5.2 Hz, 3H), 7.10 (d, J=3.6 Hz, 1H), 6.96-7.03 (m, 3H), 6.25 (d, J=8.2 Hz, 1H), 5.66-5.75 (m, 1H), 3.91-3.98 (m, 1H), 3.72 (s, 3H), 3.03-3.12 (m, 2H), 2.91-3.00 (m, 1H), 2.85 (d, J=7.2 Hz, 2H), 2.71-2.75 (m, 1H), 2.25-2.35 (m, 1H), 0.98 (d, J=6.4 Hz, 6H).

### Step E: 7-[(4-fluorophenyl)methyl]-4-[5-[[(1R)-indan-1-yl]carbamoyl]-2-thienyl]-2-isobutyl-quinoline-3-carboxylic acid

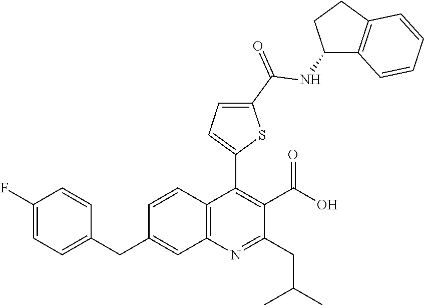 [SMILES: CC(C)Cc1nc2cc(Cc3ccc(F)cc3)ccc2c(-c2ccc(C(=O)N[C@@H]3CCc4ccccc43)s2)c1C(=O)O]

To a solution of methyl 7-[(4-fluorophenyl)methyl]-4-[5-[[(1R)-indan-1-yl]carbamoyl]-2-thienyl]-2-isobutyl-quinoline-3-carboxylate (220 mg, 371.17 μmol) in DMA (5 mL) was added LiCl (78.68 mg, 1.86 mmol). The mixture was stirred at 150° C. for 48 hours. The solution was concentrated to afford the crude. The residue was purified by flash silica gel chromatography (Eluent of 0~10% MeOH/DCM) to afford 7-[(4-fluorophenyl)methyl]-4-[5-[[(1R)-indan-1-yl]carbamoyl]-2-thienyl]-2-isobutyl-quinoline-3-carboxylic acid (180 mg, 83.80%) which was used in the next step without further purification.

### Step F: N-[(R)-1-indanyl]-5-{3-carbamoyl-7-[(p-fluorophenyl)methyl]-2-isobutyl-4-quinolyl}-2-thenamide

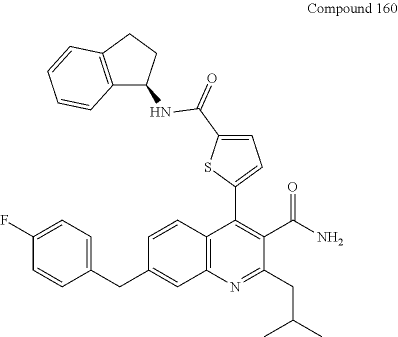 [SMILES: CC(C)Cc1nc2cc(Cc3ccc(F)cc3)ccc2c(-c2ccc(C(=O)N[C@@H]3CCc4ccccc43)s2)c1C(N)=O]

To a solution of 7-[(4-fluorophenyl)methyl]-4-[5-[[(1R)-indan-1-yl]carbamoyl]-2-thienyl]-2-isobutyl-quinoline-3-carboxylic acid (120 mg, 207.36 μmol) in DMF (1 mL) was added HATU (118.27 mg, 311.05 μmol) and DIPEA (80.40 mg, 622.09 μmol). The mixture was stirred at 25° C. for 1 hour. Then NH₄Cl (22.18 mg, 414.73 μmol) was added. The mixture was stirred at 60° C. for 15 hours. The reaction mixture was diluted with water (10 ml.) and extracted with EtOAc (5 mL×3). The combined organic layers were washed with water (10 mL×2), dried over Na₂SO₄, filtered and concentrated under reduced pressure to afford a residue. The residue was purified by flash silica gel chromatography (Eluent of 0~80% EtOAc in PE) to afford N-[(R)-1-indanyl]-5-{3-carbamoyl-7-[(p-fluorophenyl)methyl]-2-isobutyl-4-quinolyl}-2-thenamide (39.44 mg, 32.92%).

¹H NMR (400 MHz, CDCl₃) δ 7.89 (s, 1H), 7.73 (d, J=8.4 Hz, 1H), 7.53 (d, J=3.6 Hz, 1H), 7.38 (d, J=7.2 Hz, 1H), 7.33 (s, 1H), 7.29 (d, J=6.0 Hz, 2H), 7.23-7.26 (m, 1H), 7.15-7.22 (m, 3H), 6.99 (t, J=8.4 Hz, 2H), 6.37 (d, J=8.4 Hz, 1H), 5.70-5.74 (m, 1H), 5.63-5.69 (m, 1H), 5.55 (s, 1H), 4.15 (s, 2H), 3.02-3.12 (m, 1H), 2.88-2.99 (m, 3H), 2.72 (m, 1H), 2.41-2.46 (m, 1H), 1.91-2.04 (m, 1H), 1.00 (d, J=6.8 Hz, 6H), LC-MS: m/z 578.3 (M+H)⁺.

### Example 30

### (S)-7-{1-[1-(4-cyano-3-fluorophenyl)ethyl]-1H-1,7-diazainden-5-yl}-3-[(p-fluorophenyl)methyl]-5-isobutyl-3-methyl-1-oxo-2,4-diaza-6-indancarboxamide (Compound 164)

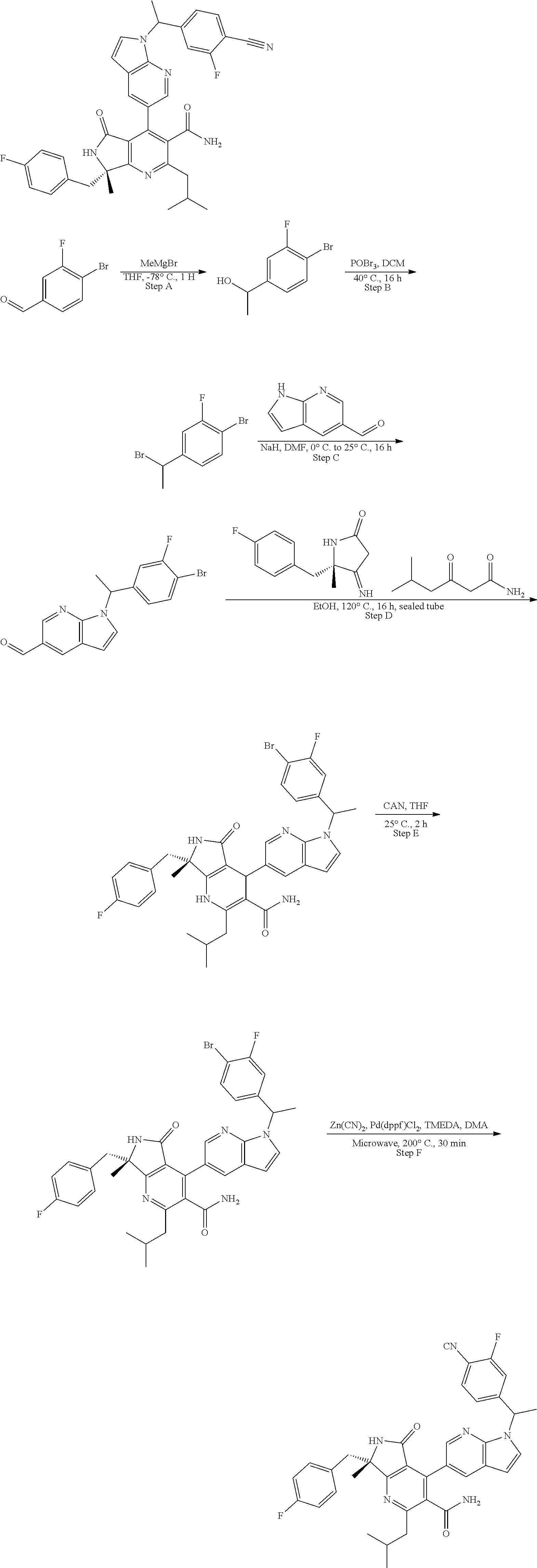

### Step A: 1-(4-bromo-3-fluorophenyl) ethan-1-ol

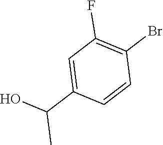

To a solution of 4-bromo-3-fluorobenzaldehyde (3.0 g, 14.778 mmol) in tetrahydrofuran (60 mL) was added methyl magnesium bromide (1M in THF, 73.888 mL) at 0° C. under N₂. The reaction mixture was stirred at 0° C. for 1 hr. The reaction mixture was quenched by aq. NH₄Cl (100 mL), the mixture was extracted with EA (300 mL), washed with brine (50 mL), dried over anhydrous Na₂SO₄, filtered and concentrated under vacuum. The resulting residue was purified by silica gel chromatography (PE/EA=5/1) to afford 1-(4-bromo-3-fluorophenyl) ethan-1-ol (1600 mg, 49.43%). ¹H NMR (400 MHz, CDCl₃) δ 7.50 (dd, J=8.0, 7.2 Hz, 1H), 7.16 (dd, J=9.6, 2.0 Hz, 1H), 7.02 (dd, J=8.0, 2.0 Hz, 1H), 4.86-4.88 (m, 1H), 1.91 (q, J=3.6 Hz, 1H), 1.47 (d, J=6.4 Hz, 3H).

### Step B: 1-bromo-4-(1-bromoethyl)-2-fluorobenzene

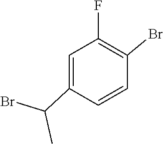

To a solution of 1-(4-bromo-3-fluorophenyl) ethan-1-ol (1.6 g, 7.304 mmol) in DCM (50 mL) were added triphenylphosphane (2.87 g, 10.956 mmol) and NBS (1.95 g, 10.956 mmol), the reaction mixture was stirred at 25° C. for 16 hrs. The reaction mixture was concentrated under vacuum. The resulting residue was purified by silica gel chromatography (PE/EA=5/1) to afford 1-bromo-4-(1-bromoethyl)-2-fluorobenzene (1600 mg, 77.69%). ¹H NMR (400 MHz, CDCl₃) δ 7.51 (t, J=8.0 Hz, 1H), 7.21 (dd, J=9.6, 2.0 Hz, 1H), 7.09 (dd, J=8.4, 2.0 Hz, 1H), 5.08-5.16 (m, 1H), 2.00 (d, J=6.8 Hz, 3H).

### Step C: 1-(1-(4-bromo-3-fluorophenyl)ethyl)-1H-pyrrolo[2,3-b]pyridine-5-carbaldehyde

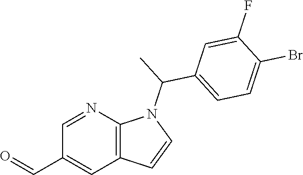 [SMILES: CC(c1ccc(Br)c(F)c1)n1ccc2cc(C=O)cnc21]

To a solution of 1H-pyrrolo[2,3-b]pyridine-5-carbaldehyde (50 mg, 0.342 mmol) in DMF (15 mL) were added 1-bromo-4-(1-bromoethyl)-2-fluorobenzene (96.43 mg, 0,342 mmol) and potassium carbonate (141.84 mg, 1.026 mmol) at 25° C. under N₂. The reaction mixture was stirred at 50° C. for 16 hrs. The reaction mixture was quenched by NH₄Cl (30 mL), extracted with EA (30 mL×3), the combined organic layers were washed with brine (50 mL×2), dried over anhydrous Na₂SO₄, filtered and concentrated under vacuum. The resulting residue was purified by prep-TLC (PE/EA=5/1) to afford 1-(1-(4-bromo-3-fluorophenyl)ethyl)-1H-pyrrolo[2,3-b]pyridine-5-carbaldehyde (110 mg, 92.61%). LC-MS: m/z 346.9 (M+H)⁺.

### Step D: (7S)-4-(1-(1-(4-bromo-3-fluorophenyl)ethyl)-1H-pyrrolo[2,3-b]pyridin-5-yl)-7-(4-fluorobenzyl)-2-isobutyl-7-methyl-5-oxo-4,5,6,7-tetrahydro-1H-pyrrolo[3,4-b]pyridine-3-carboxamide

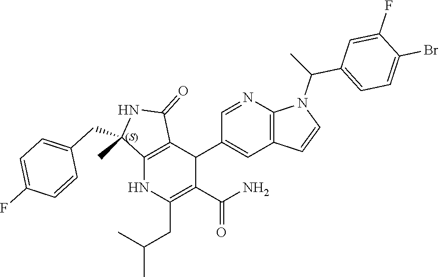

To a solution of(S)-5-(4-fluorobenzyl)-4-imino-5-methylpyrrolidin-2-one (50 mg, 0,227 mmol) in EtOH (5 mL) were added 1-(1-(4-bromo-3-fluorophenyl)ethyl)-1H-pyrrolo[2,3-b]pyridine-5-carbaldehyde (78.81 mg, 0.227 mmol) and 5-methyl-3-oxohexanamide (130.02 mg, 0.908 mmol). The reaction mixture was stirred at 120° C. for 16 hrs under N₂ in a seal tube. The reaction mixture was concentrated under vacuum to afford (7S)-4-(1-(1-(4-bromo-3-fluorophenyl)ethyl)-1H-pyrrolo[2,3-b]pyridin-5-yl)-7-(4-fluorobenzyl)-2-isobutyl-7-methyl-5-oxo-4,5,6,7-tetrahydro-1H-pyrrolo[3,4-b]pyridine-3-carboxamide (150 mg, 97.96%) which was used in the next step without further purification. LC-MS: m/z 675.9 (M+H)⁺.

### Step E: (7S)-4-(1-(1-(4-bromo-3-fluorophenyl)ethyl)-1H-pyrrolo[2,3-b]pyridin-5-yl)-7-(4-fluorobenzyl)-2-isobutyl-7-methyl-5-oxo-6,7-dihydro-5H-pyrrolo[3,4-b]pyridine-3-carboxamide

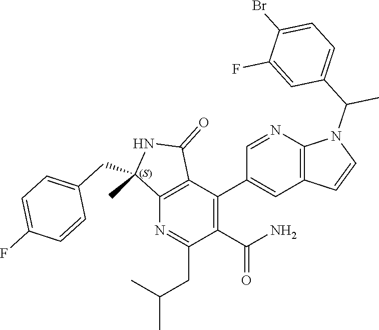

To a solution of (7S)-4-(1-(1-(4-bromo-3-fluorophenyl)ethyl)-1H-pyrrolo[2,3-b]pyridin-5-yl)-7-(4-fluorobenzyl)-2-isobutyl-7-methyl-5-oxo-4,5,6,7-tetrahydro-1H-pyrrolo[3,4-b]pyridine-3-carboxamide (150 mg, 0.222 mmol) in tetrahydrofuran (10 mL) was added CAN (259.81 mg, 0.445 mmol) at 25° C. under N₂. The reaction mixture was stirred at 25° C. for 2 hrs. The reaction mixture was quenched by aq. NaHCO₃ (20 mL), the mixture was extracted with EA (30 mL), washed with brine (30 mL), dried over anhydrous Na₂SO₄, filtered and concentrated in vacuum. The resulting residue was purified by prep-TLC (DCM/MeOH=10/1) to afford (7S)-4-(1-(1-(4-bromo-3-fluorophenyl)ethyl)-1H-pyrrolo[2,3-b]pyridin-5-yl)-7-(4-fluorobenzyl)-2-isobutyl-7-methyl-5-oxo-6,7-dihydro-5H-pyrrolo[3,4-b]pyridine-3-carboxamide (48 mg, 30.08%) as a yellow oil. LC-MS: m/z 674.2 (M+H)⁺.

### Step F: (S)-7-{1-[1-(4-cyano-3-fluorophenyl)ethyl]-1H-1,7-diazainden-5-yl}-3-[(p-fluorophenyl)methyl]-5-isobutyl-3-methyl-1-oxo-2,4-diaza-6-indancarboxamide (Compound 164)

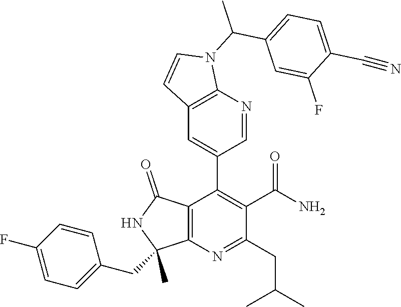 [SMILES: CC(C)Cc1nc2c(c(-c3cnc4c(ccn4C(C)c4ccc(C#N)c(F)c4)c3)c1C(N)=O)C(=O)N[C@@]2(C)Cc1ccc(F)cc1]

To a solution of (7S)-4-(1-(1-(4-bromo-3-fluorophenyl)ethyl)-1H-pyrrolo[2,3-b]pyridin-5-yl)-7-(4-fluorobenzyl)-2-isobutyl-7-methyl-5-oxo-6,7-dihydro-5H-pyrrolo[3,4-b]pyridine-3-carboxamide (22 mg, 0.033 mmol) in DMA (5 mL) was added Pd(dppf)Cl₂ (2.37 mg, 0.003 mmol), TMEDA (1.90 mg, 0.016 mmol) and Zn(CN)₂ (11.52 mg, 0.098 mmol). The reaction mixture was stirred at 200° C. for 30 min by microwave. After the reaction was completed, water (10 mL) was added, the mixture was extracted with EA (10 mL×3), the combined organic layers were washed with brine (15 mL), dried over anhydrous Na₂SO₄, filtered and concentrated under vacuum. The resulting residue was purified by prep-HPLC (Waters 3767/Qda Column: SunFire Sunfire C18, 19*250 mm*10 μm; Mobile Phase A: 0.1% FA/H2O, B: ACN; Flow rate: 40 ml/min; Gradient: 46%; Retention Time: 9.2-10.1 min of 17 min) to afford (S)-7-{1-[1-(4-cyano-3-fluorophenyl)ethyl]-1H-1,7-diazainden-5-yl}-3-[(p-fluorophenyl)methyl]-5-isobutyl-3-methyl-1-oxo-2,4-diaza-6-indancarboxamide (1.9 mg, 8.75%).

¹H NMR (400 MHz, DMSO-d₆) δ 8.70 (s, 1H), 7.85-7.90 (m, 3H), 7.71-7.74 (m, 2H), 7.52 (dd, J=10.0, 3.6 Hz, 1H), 7.42 (s, 1H), 7.29 (t, J=7.2 Hz, 1H), 6.92 (t, J=8.8 Hz, 2H), 6.83-6.85 (m, 2H), 6.59 (d, J=3.2 Hz, 1H), 6.22-6.27 (m, 1H), 3.26 (d, J=13.6 Hz, 1H), 3.06 (d. J=13.6 Hz, 1H), 2.76-2.81 (m, 2H), 2.36-2.40 (m, 1H), 1.91 (d, J=7.2 Hz, 3H), 1.57 (s, 3H), 1.03 (d, J=6.8 Hz, 3H), 0.93 (dd, J=6.8, 2.8 Hz, 3H). ¹⁹F NMR (376 MHz, DMSO-d₆) δ −107.96, −116.65. LC-MS: m/z 619.3 (M+H)⁺.

### (S)-7-{1-[(S)-1-(4-cyano-3-fluorophenyl)ethyl]-1H-1,7-diazainden-5-yl}-3-[(p-fluorophenyl)methyl]-5-isobutyl-3-methyl-1-oxo-2,4-diaza-6-indancarboxamide(S)-7-{1-[(R)-1-(4-cyano-3-fluorophenyl)ethyl]-1H-1,7-diazainden-5-yl}-3-[(p-fluorophenyl)methyl]-5-isobutyl-3-methyl-1-oxo-2,4-diaza-6-indancarboxamide (Compound 162 and Compound 163)

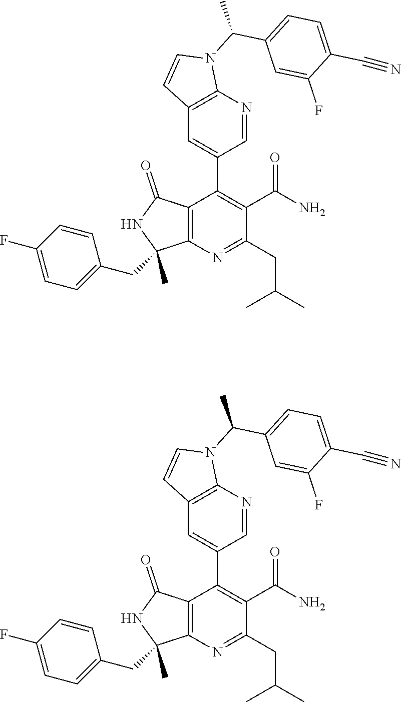 [SMILES: CC(C)Cc1nc2c(c(-c3cnc4c(ccn4[C@H](C)c4ccc(C#N)c(F)c4)c3)c1C(N)=O)C(=O)N[C@@]2(C)Cc1ccc(F)cc1 ; CC(C)Cc1nc2c(c(-c3cnc4c(ccn4[C@@H](C)c4ccc(C#N)c(F)c4)c3)c1C(N)=O)C(=O)N[C@@]2(C)Cc1ccc(F)cc1]

(S)-7-{1-[1-(4-cyano-3-fluorophenyl)ethyl]-1H-1,7-diazainden-5-yl}-3-[(p-fluorophenyl)methyl]-5-isobutyl-3-methyl-1-oxo-2,4-diaza-6-indancarboxamide (31.0 mg, 0.047 mmol) was separated by SFC (system: Waters SFC 80; Column: DAICEL CHIRALPAK® IC; Column size: 250*25 mm 10 μm; Mobile Phase A: Supercritical CO₂; Mobile Phase B: MeOH (+0.1% 7.0 mol/L Ammonia in MEOH); Gradient: A/B=60/40; Flow rate: 70 mL/min; Column Temp: 25° C.) to afford (S)-7-{1-[(S)-1-(4-cyano-3-fluorophenyl)ethyl]-1H-1,7-diazainden-5-yl}-3-[(p-fluorophenyl)methyl]-5-isobutyl-3-methyl-1-oxo-2,4-diaza-6-indancarboxamide and (S)-7-{1-[(R)-1-(4-cyano-3-fluorophenyl)ethyl]-1H-1,7-diazainden-5-yl}-3-[(p-fluorophenyl)methyl]-5-isobutyl-3-methyl-1-oxo-2,4-diaza-6-indancarboxamide.

Compound 162, enantiomer 1 (7.6 mg, 21.2%): Rt=2.67 min. ¹H NMR (400 MHz, DMSO-d₆) & 8.70 (s, 1H), 7.84-7.90 (m, 3H), 7.72 (s, 2H), 7.50 (dd, J=10.4, 1.2 Hz, 1H), 7.42 (s, 1H), 7.27 (dd, J=8.4, 1.2 Hz, 1H), 6.91 (t, J=8.8 Hz, 2H), 6.83-6.85 (m, 2H), 6.58 (d, J=3.6 Hz, 1H), 6.21-6.26 (m, 1H), 3.25 (d, J=13.6 Hz, 1H), 3.05 (d, J=13.6 Hz, 1H), 2.73-2.80 (m, 2H), 2.34-2.42 (m, 1H), 1.91 (d, J=7.2 Hz, 3H), 1.56 (s, 3H), 1.02 (d, J=6.8 Hz, 3H), 0.92 (d, J=6.8 Hz, 3H). ¹⁹F NMR (376 MHz, DMSO-d₆) δ −107.96, −116.65. LC-MS: m/z 619.3 (M+H)⁺.

Compound 163, enantiomer 2 (7.45 mg, 20.8%): Rt=1.63 min. ¹H NMR (400 MHz, DMSO-d₆) δ 8.70 (s, 1H), 7.85-7.90 (m, 3H), 7.71-7.74 (m, 2H), 7.52 (d, J=9.6 Hz, 1H), 7.42 (s, 1H), 7.29 (dd, J=8.0, 1.2 Hz, 1H), 6.91 (t, J=8.8 Hz, 2H), 6.82-6.86 (m, 2H), 6.58 (d, J=3.6 Hz, 1H), 6.21-6.26 (m, 1H), 3.25 (d, J=13.2 Hz, 1H), 3.06 (d, J=13.2 Hz, 1H), 2.72-2.83 (m, 2H), 2.36-2.40 (m, 1H), 1.91 (d, J=7.2 Hz, 3H), 1.56 (s, 3H), 1.03 (d, J=6.8 Hz, 3H), 0.93 (d, J=6.8 Hz, 3H). ¹⁹F NMR (376 MHz, DMSO-d₆) δ −107.93, −116.64. LC-MS: m/z 619.3 (M+H)⁺.

### Example 31

### 7-{1-[(3,4-difluorophenyl)methyl]-5-indolyl}-3-[(p-fluorophenyl)methyl]-5-isobutyl-3-methyl-1,1-dioxo-1λ⁶-thia-4-aza-6-indancarboxamide (Compound 161)

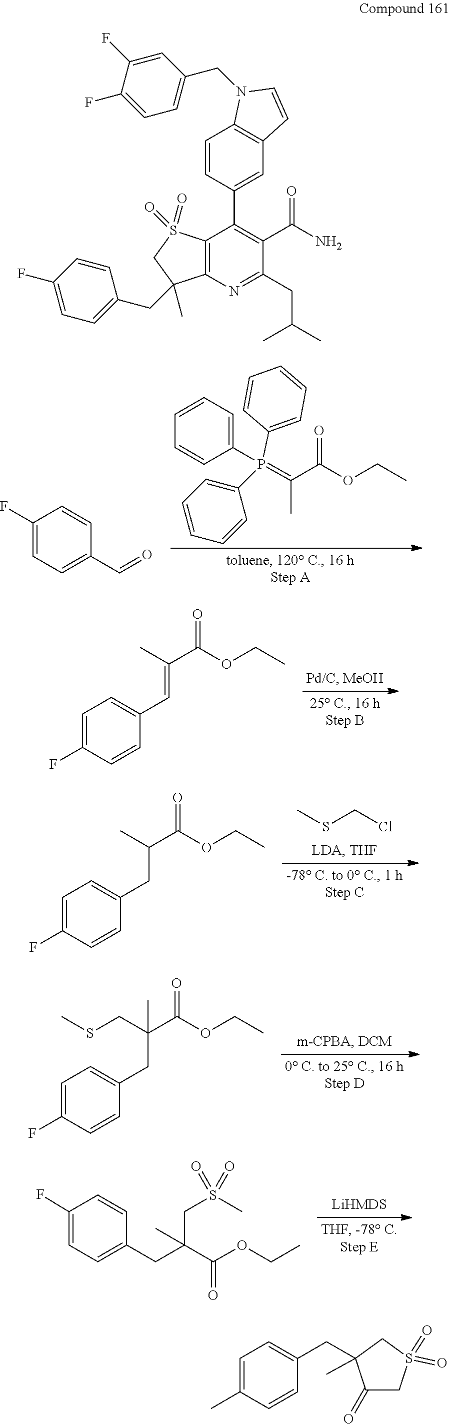

### Step A: ethyl (E)-3-(4-fluorophenyl)-2-methylacrylate

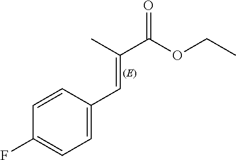

To a solution of 4-fluorobenzene-1-carbaldehyde (5 g, 40.287 mmol) in Toluene (200 mL) were added ethyl 2-(triphenyl-λ⁵-phosphanylidene) propanoate (17.52 g, 48.344 mmol), the reaction mixture was stirred at 120° C. for 3 hrs under N₂. The reaction mixture was concentrated under vacuum, the resulting residue was purified by silica gel chromatography (eluting PE/EA=10/1) to afford ethyl (E)-3-(4-fluorophenyl)-2-methylacrylate (9.5 g, 100%). ¹H NMR (400 MHz, CDCl₃) δ 7.56 (s, 1H), 7.29-7.32 (m, 2H), 6.98-7.03 (m, 2H), 4.17-4.22 (m, 2H), 2.02 (d, J=1.2 Hz, 3H), 1.27 (t, J=7.2 Hz, 3H).

### Step B: ethyl 3-(4-fluorophenyl)-2-methylpropanoate

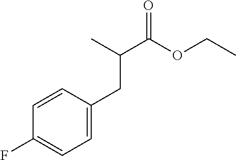

To a solution of ethyl (E)-3-(4-fluorophenyl)-2-methylacrylate (9.5 g, 45.623 mmol) in MeOH (250 mL) was added 10% Pd/C (0.97 g, 9.125 mmol). The reaction mixture was stirred at room temperature for 18 hrs under H₂ balloon. The reaction mixture was filtered by Celite, the filtrate was concentrated in vacuum to afford ethyl 3-(4-fluorophenyl)-2-methylpropanoate (9.0 g, 93.83%) which was used in the next step without further purification. ¹H NMR (400 MHz, CDCl₃) δ 7.03-7.06 (m, 2H), 6.86-6.90 (m, 2H), 3.98-4.03 (m, 2H), 2.87-2.90 (m, 1H), 2.55-2.63 (m, 2H), 1.11 (t, J=7.2 Hz, 3H), 1.08 (d, J=6.8 Hz, 3H).

### Step C: ethyl 2-(4-fluorobenzyl)-2-methyl-3-(methylthio) propanoate

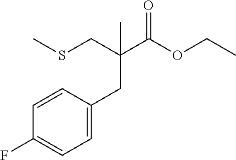 [SMILES: CCOC(=O)C(C)(CSC)Cc1ccc(F)cc1]

To a solution of ethyl 3-(4-fluorophenyl)-2-methylpropanoate (2.8 g, 13.317 mmol) in THF (50 mL) was added LDA (13.317 mL, 26.635 mmol) at −65° C. under N₂, the reaction mixture was stirred at −65° C. for 30 min, (chloromethyl)(methyl)sulfane (3.86 g, 39.952 mmol) was added. The reaction mixture was allowed to warm to room temperature and stirred for another 16 hrs under N₂. The reaction mixture was quenched by aq. NH₄Cl (30 mL), the mixture was extracted with EA (50 mL×2), the combined organic layers were washed with brine, dried over anhydrous Na₂SO₄, filtered and concentrated under vacuum. The resulting residue was purified by silica gel chromatography (eluting PE/EA=10/1) to afford ethyl 2-(4-fluorobenzyl)-2-methyl-3-(methylthio) propanoate (2.24 g, 62.21%). ¹H NMR (400 MHz, CDCl₃) δ 7.01-7.04 (m, 2H), 6.85-6.89 (m, 2H), 4.03-4.08 (m, 2H), 2.91 (d, J=13.2 Hz, 1H), 2.74-2.78 (m, 2H), 2.56 (d, J=12.8 Hz, 1H), 2.06 (s, 3H), 1.17 (t, J=7.2 Hz, 3H), 1.14 (s, 3H).

### Step D: ethyl 2-(4-fluorobenzyl)-2-methyl-3-(methylsulfonyl)propanoate

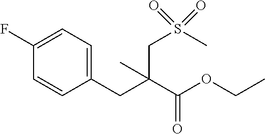 [SMILES: CCOC(=O)C(C)(Cc1ccc(F)cc1)CS(C)(=O)=O]

To a solution of ethyl 2-(4-fluorobenzyl)-2-methyl-3-(methylthio) propanoate (2.24 g, 8.285 mmol) in DCM (30 mL) was added m-CPBA (8.41 g, 41.425 mmol) at 0° C. under N₂. The reaction mixture was stirred at room temperature for 16 hrs. The mixture was filtered, the filtrate was washed with 1N NaOH (50 mL), the organic layer was washed with water (50 mL) and brine (50 mL), dried over anhydrous Na₂SO₄, filtered and concentrated under vacuum. The resulting residue was purified by silica gel chromatography (eluting PE/EA=2/1) to afford ethyl 2-(4-fluorobenzyl)-2-methyl-3-(methylsulfonyl) propanoate (1.4 g, 55.89%). ¹H NMR (400 MHz, CDCl₃) δ 7.09-7.12 (m, 2H), 6.95-7.01 (m, 2H), 4.11-4.20 (m, 2H), 3.60 (d, J=14.4 Hz, 1H), 3.13 (d, J=14.4 Hz, 1H), 3.03 (d, J=13.6 Hz, 1H), 2.92-2.96 (m, 4H), 1.47 (s, 3H), 1.22-1.27 (m, 3H). LC-MS: m/z 324.8 (M+Na)⁺.

### Step E: 4-(4-fluorobenzyl)-4-methyldihydrothiophen-3(2H)-one 1,1-dioxide

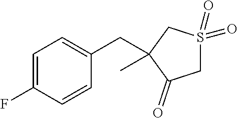 [SMILES: CC1(Cc2ccc(F)cc2)CS(=O)(=O)CC1=O]

To a solution of ethyl 2-(4-fluorobenzyl)-2-methyl-3-(methylsulfonyl)propanoate (1.4 g, 4,630 mmol) in THF (45 mL) was added 2-(methoxymethyl) oxiran-1-ium (23.151 mL, 23.151 mmol), the mixture was stirred at −78° C. for 1 hr under N₂. The reaction mixture was concentrated in vacuum. The resulting residue was purified by silica gel chromatography (eluting PE/EA=4/1) to afford 4-(4-fluorobenzyl)-4-methyldihydrothiophen-3(2H)-one 1,1-dioxide (1.1 g, 92.70%). ¹H NMR (400 MHz, CDCl₃) δ 6.94-7.06 (m, 4H), 3.73 (dd, J=18.0, 1.6 Hz, 1H), 3.48-3.56 (m, 2H), 3.20 (d, J=14.0 Hz, 1H), 3.08 (dd, J=14.4, 2.4 Hz, 1H), 2.68 (d, J=14.0 Hz, 1H), 1.36 (s, 3H).

### Step F: 7-{1-[(3,4-difluorophenyl)methyl]-5-indolyl}-3-[(p-fluorophenyl)methyl]-5-isobutyl-3-methyl-1,1-dioxo-1λ⁶-thia-4-aza-6-indancarboxamide (Compound 161)

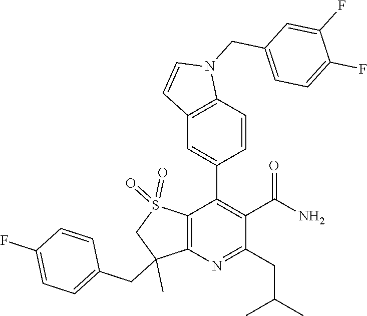 [SMILES: CC(C)Cc1nc2c(c(-c3ccc4c(ccn4Cc4ccc(F)c(F)c4)c3)c1C(N)=O)S(=O)(=O)CC2(C)Cc1ccc(F)cc1]

Compound 161 was then synthesized following the similar route of Example 2, using 4-(4-fluorobenzyl)-4-methyldihydrothiophen-3(2H)-one 1,1-dioxide and 5-methyl-3-oxohexanamide in step B.

¹H NMR (400 MHz, CD₃OD) δ 7.78 (s, 1H), 7.35-7.38 (m, 2H), 7.30 (dd, J=8.8, 1.2 Hz, 1H), 7.12-7.19 (m, 3H), 7.05-7.09 (m, 1H), 6.97-7.01 (m, 3H), 6.56 (d, J=3.2 Hz, 1H), 5.39 (s, 2H), 3.59 (d, J=13.6 Hz, 1H), 3.42 (d, J=13.6 Hz, 1H), 3.27 (d, J=13.6 Hz, 1H), 3.14 (d, J=13.6 Hz, 1H), 2.87-2.90 (m, 2H), 2.41-2.45 (m, 1H), 1.57 (s, 3H), 1.03 (t, J=6.4 Hz, 6H). ¹⁹F NMR (376 MHz, CD₃OD) δ −118.06, −140.11, −142.51. LC-MS: m/z 632.0 (M+H)⁺.

### Example 32

### (S)-7-{1-[(3,4-difluorophenyl)methyl]-5-indolinyl}-3-[(p-fluorophenyl)methyl]-5-isobutyl-3-methyl-1-oxo-2,4-diaza-6-indancarboxamide (Compound 158)

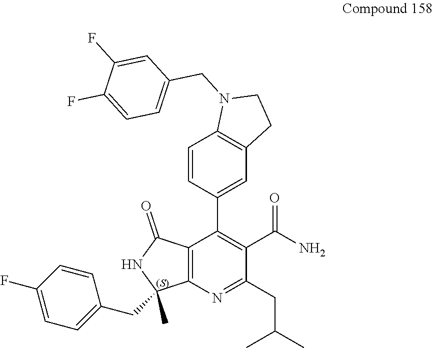

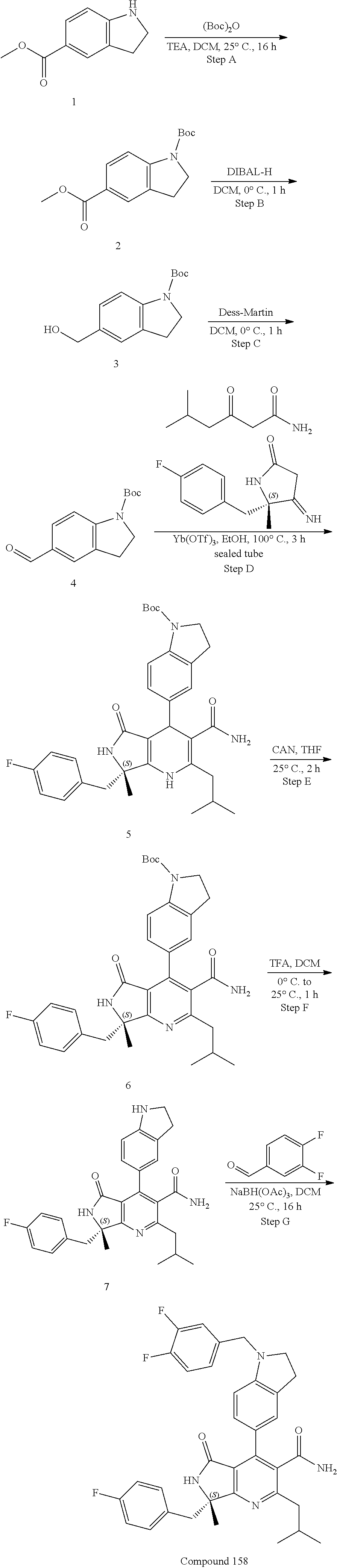

### Step A: 1-(tert-butyl) 5-methyl indoline-1,5-dicarboxylate

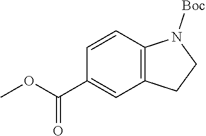 [SMILES: COC(=O)c1ccc2c(c1)CCN2C(=O)OC(C)(C)C]

To a solution of methyl 2,3-dihydro-1H-indole-5-carboxylate (500 mg, 2.822 mmol) in DCM (20 mL) were added (2-methylprop-2-yl)oxidanecarboxylic anhydride (0.779 mL, 3.386 mmol) and TEA (0.471 mL, 3.386 mmol) at 25° C. under N₂. The reaction mixture was stirred at 25° C. for 16 hrs. The reaction mixture was concentrated under vacuum. The resulting residue was purified by TLC (PE/EA=10/1) to afford 1-(tert-butyl) 5-methyl indoline-1,5-dicarboxylate (700 mg, 89.46%). LC-MS: m/z 278.2 (M+H)⁺.

### Step B: tert-butyl 5-(hydroxymethyl) indoline-1-carboxylate

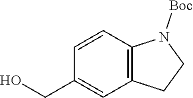 [SMILES: CC(C)(C)OC(=O)N1CCc2cc(CO)ccc21]

To a solution of 1-(tert-butyl) 5-methyl indoline-1,5-dicarboxylate (700 mg, 2.524 mmol) in DCM (20 mL) was added DIBAL-H (3.366 mL, 5.048 mmol) at 0° C. under N₂. The reaction mixture was stirred at 0° C. for 1 hr. The reaction mixture was quenched by 30% aq. citric acid (10 mL), extracted with DCM (20 mL×2), the combined organic layers were concentrated under vacuum to afford tert-butyl 5-(hydroxymethyl) indoline-1-carboxylate (620 mg, 98.52%) which was used in the next step without further purification. LC-MS: m/z 194.0 (M+H-^{t}Bu)⁻.

### Step C: tert-butyl 5-formylindoline-1-carboxylate

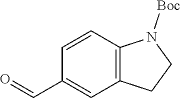 [SMILES: CC(C)(C)OC(=O)N1CCc2cc(C=O)ccc21]

To a solution of tert-butyl 5-(hydroxymethyl) indoline-1-carboxylate (620 mg, 2.487 mmol) in DCM (20 mL) was added DMP (2109.56 mg, 4.974 mmol) at 0° C. under N₂. The reaction mixture was stirred at 25° C. for 2 hrs. The reaction mixture was quenched by aq. NaHCO₃ (10 mL), extracted with DCM (20 mL×2), the combined organic layers were concentrated under vacuum. The resulting residue was purified by prep-TLC (PE/EA=10/1) to afford tert-butyl 5-formylindoline-1-carboxylate (100 mg, 16.26%). LC-MS: m/z 248.0 (M+H)⁺.

### Step D: tert-butyl 5-((7S)-3-carbamoyl-7-(4-fluorobenzyl)-2-isobutyl-7-methyl-5-oxo-4,5,6,7-tetrahydro-1H-pyrrolo[3,4-b]pyridin-4-yl) indoline-1-carboxylate

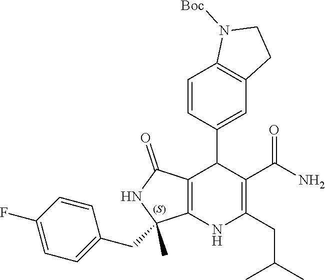

To a solution of(S)-5-(4-fluorobenzyl)-4-imino-5-methylpyrrolidin-2-one (44.53 mg, 0.202 mmol) in EtOH (5 mL) were added tert-butyl 5-formylindoline-1-carboxylate (100 mg, 0.404 mmol), 5-methyl-3-oxohexanamide (86.86 mg, 0.607 mmol) and Yb(OTf)₃ (12.54 mg, 0.020 mmol). The reaction mixture was stirred at 100° C. for 3 hrs under N₂ in a sealed tube. The reaction mixture was concentrated in vacuum to afford tert-butyl 5-((7S)-3-carbamoyl-7-(4-fluorobenzyl)-2-isobutyl-7-methyl-5-oxo-4,5,6,7-tetrahydro-1H-pyrrolo[3,4-b]pyridin-4-yl) indoline-1-carboxylate (116 mg, 99.83%) was used in the next step without further purification. LC-MS: m/z 575.2 (M+H)⁺.

### Step E: tert-butyl(S)-5-(3-carbamoyl-7-(4-fluorobenzyl)-2-isobutyl-7-methyl-5-oxo-6,7-dihydro-5H-pyrrolo[3,4-b]pyridin-4-yl) indoline-1-carboxylate

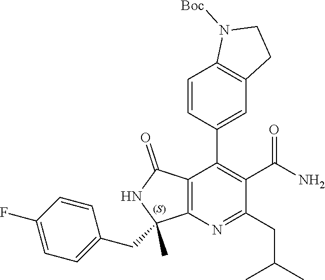

To a solution of tert-butyl 5-((7S)-3-carbamoyl-7-(4-fluorobenzyl)-2-isobutyl-7-methyl-5-oxo-4,5,6,7-tetrahydro-1H-pyrrolo[3,4-b]pyridin-4-yl) indoline-1-carboxylate (139 mg, 0.227 mmol) in THF (10 mL) was added CAN (235.84 mg, 0.404 mmol) at 25° C. under N₂. The reaction mixture was stirred at 25° C. for 2 hrs. The reaction mixture was quenched by NaHCO₃ (10 mL), extracted with EtOAc (30 mL), washed with brine (30 mL), dried over Na₂SO₄, filtered and concentrated under vacuum. The resulting residue was purified by TLC (DCM/MeOH=10/1) to afford tert-butyl(S)-5-(3-carbamoyl-7-(4-fluorobenzyl)-2-isobutyl-7-methyl-5-oxo-6,7-dihydro-5H-pyrrolo[3,4-b]pyridin-4-yl) indoline-1-carboxylate (72 mg, 62.29%). LC-MS: m/z 573.8 (M+H)⁺.

### Step F: 1-(3,4-difluorobenzyl)imidazo[1,5-a]pyridine-6-carbaldehyde

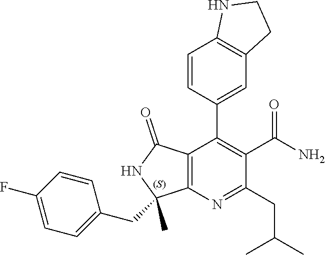

To a solution of tert-butyl(S)-5-(3-carbamoyl-7-(4-fluorobenzyl)-2-isobutyl-7-methyl-5-oxo-6,7-dihydro-5H-pyrrolo[3,4-b]pyridin-4-yl) indoline-1-carboxylate (72 mg, 0.126 mmol) in DCM (10 mL) was added TFA (1 mL) at 25° C. under N₂. The reaction mixture was stirred at 25° C. for 2 hrs. The mixture was concentrated under vacuum to afford (S)-7-(4-fluorobenzyl)-4-(indolin-5-yl)-2-isobutyl-7-methyl-5-oxo-6,7-dihydro-5H-pyrrolo[3,4-b]pyridine-3-carboxamide (59.41 mg, 100%) which was used in the next step without further purification. LC-MS: m/z 473.7 (M−H)⁺.

### Step G: (S)-7-{1-[(3,4-difluorophenyl)methyl]-5-indolinyl}-3-[(p-fluorophenyl)methyl]-5-isobutyl-3-methyl-1-oxo-2,4-diaza-6-indancarboxamide (Compound 158)

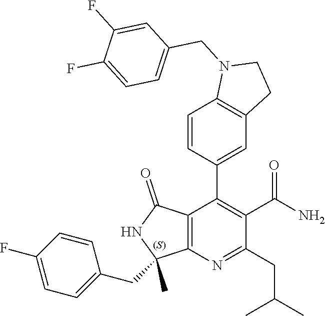

To a solution of(S)-7-(4-fluorobenzyl)-4-(indolin-5-yl)-2-isobutyl-7-methyl-5-oxo-6,7-dihydro-5H-pyrrolo[3,4-b]pyridine-3-carboxamide (25 mg, 0.053 mmol) in DCM (5 mL) was added 3,4-difluorobenzene-1-carbaldehyde (0.012 mL, 0.106 mmol) at 25° C. under N₂. The reaction mixture was stirred at 25° C. for 10 mins, NaBH(OAc)₃ (56.06 mg, 0.265 mmol) and acetic acid (0.003 mL, 0.053 mmol) were added. The reaction mixture was stirred at 25° C. for another 16 hrs. The reaction mixture was concentrated under vacuum. The resulting residue was purified by prep-TLC (DCM/MeOH=20/1) then prep-HPLC (Waters 3767/QDA Column: SunFire C18, 19*250 mm, 10 μm; Mobile Phase A: 10 mmol/L A: 0.05% TFA/H₂O, B: CAN; flow rate: 20 mL/min; Gradient: 55-60% Retention Time: 8.0-9.5 min of 17 min) to afford (S)-7-{1-[(3,4-difluorophenyl)methyl]-5-indolinyl}-3-[(p-fluorophenyl)methyl]-5-isobutyl-3-methyl-1-oxo-2,4-diaza-6-indancarboxamide (4.5 mg, 14.12%).

¹H NMR (400 MHz, DMSO-d₆) δ 8.55 (s, 1H), 7.57 (s, 1H), 7.44-7.37 (m, 3H), 7.23-7.20 (m, 1H), 6.92-6.82 (m, 6H), 6.47 (d, J=8.4 Hz, 1H), 4.27 (s, 2H), 3.32-3.29 (m, 1H), 3.22 (d, J=13.6 Hz, 1H), 3.02 (d, J=13.6 Hz, 1H), 2.85 (t, J=8.0 Hz, 2H), 2.78-2.68 (m, 2H), 2.54-2.50 (m, 1H), 2.40-2.33 (m, 1H), 1.52 (s, 3H), 1.01 (d, J=6.4 Hz, 3H), 0.91 (d, J=6.4 Hz, 3H). ¹⁹F NMR (377 MHz, DMSO-d₆) δ −116.82, −138.75, −141.14. LC-MS: m/z 599.4 (M+H)⁺.

### Example 33

### (S)-7-{1-[(3,4-difluorophenyl)methyl]-2,3a-diaza-5-indenyl}-3-[(p-fluorophenyl)methyl]-5-isobutyl-3-methyl-1-oxo-2,4-diaza-6-indancarboxamide (Compound 159)

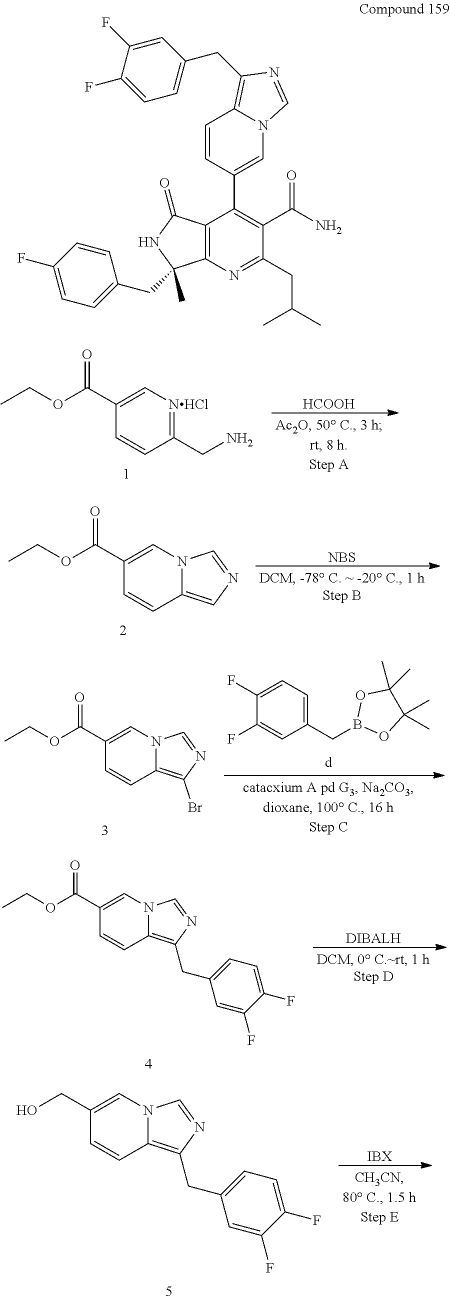

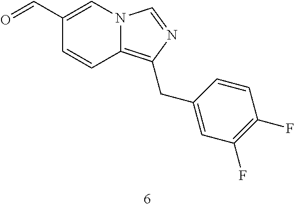 [SMILES: O=Cc1ccc2c(Cc3ccc(F)c(F)c3)ncn2c1]

### Step A: ethyl imidazo[1,5-a]pyridine-6-carboxylate

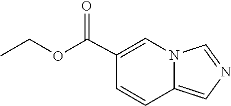

To a solution of FA (3 mL) in AC₂O (7 mL) was stirred at 50° C. for 3 h under N₂. Then ethyl 6-(aminomethyl)nicotinate.HCl (500 mg, 2.308 mmol) was added. The mixture was stirred at room temperature for 8 hrs under N₂ atmosphere. The mixture was poured into water (50 mL) and extracted with EtOAc (2×50 mL), the combined organic layer was washed with brine, dried over Na₂SO₄, filtered and the residue was purified by column chromatography (DCM/MeOH=20/1) on silica gel to obtain ethyl imidazo[1,5-a]pyridine-6-carboxylate (362 mg, 82.48%). LC-MS: m/z 191.1 (M+H)⁺.

### Step B: ethyl 1-bromoimidazo[1,5-a]pyridine-6-carboxylate

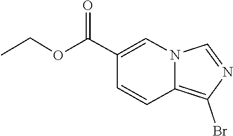

A mixture of ethyl imidazo[1,5-a]pyridine-6-carboxylate (240 mg, 1.262 mmol) and NBS (224.59 mg, 1.262 mmol) in DCM (8 mL) was stirred at −78° C. to −20° C. for 1 hr under N₂ atmosphere. The reaction mixture was diluted with EtOAc (50 mL) and saturated Na₂CO₃ solution (50 mL). The organic layer was separated, washed with further brine (50 mL×2), dried over Na₂SO₄ and concentrated in vacuo. The residue was purified using silica gel column chromatography eluting with (DCM:MeOH=20:1). The residue was concentrated in vacuo to afford ethyl 1-bromoimidazo[1,5-a]pyridine-6-carboxylate (271 mg, 79.81%). LC-MS: m/z 269.1 (M+H)⁺.

### Step C: ethyl 1-(3,4-difluorobenzyl)imidazo[1,5-a]pyridine-6-carboxylate

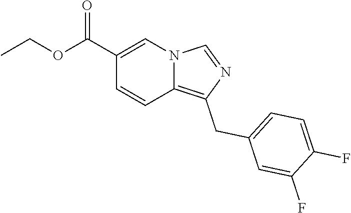 [SMILES: CCOC(=O)c1ccc2c(Cc3ccc(F)c(F)c3)ncn2c1]

A mixture of ethyl 1-bromoimidazo[1,5-a]pyridine-6-carboxylate (271 mg, 1.007 mmol), 2-(3,4-difluorobenzyl)-4,4,5,5-tetramethyl-1,3,2-dioxaborolane (511.75 mg, 2.014 mmol), catacxium A pd G3 (13.53 mg, 0.019 mmol) and Na₂CO₃ (2 mL) in dioxane (8 mL) was stirred at 100° C. for 16 hrs under N₂ atmosphere. The mixture was poured into water (50 mL) and extracted with EtOAc (2×50 mL), the combined organic layer was washed with brine, dried over Na₂SO₄, filtered and the residue was purified by column chromatography (PE/EtOAc=3/1) on silica gel to obtain ethyl 1-(3,4-difluorobenzyl)imidazo[1,5-a]pyridine-6-carboxylate (160 mg, 50.23%). LC-MS: m/z 317.2 (M+H)⁺.

### Step D: (1-(3,4-difluorobenzyl)imidazo[1,5-a]pyridin-6-yl) methanol

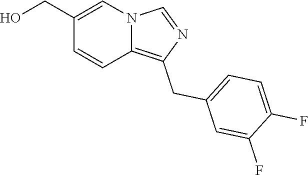 [SMILES: OCc1ccc2c(Cc3ccc(F)c(F)c3)ncn2c1]

To a solution of ethyl 1-(3,4-difluorobenzyl)imidazo[1,5-a]pyridine-6-carboxylate (100 mg, 0.316 mmol) in DCM (4 mL) was added a solution of DIBAL-H (1 M in toluene) (62.95 mg, 0.443 mmol) dropwise at 0° C. under Ar for 1 hr. The reaction mixture was diluted with DCM (50 mL) and citric Acid (30% in H₂O) (1.26 mL). The organic layer was separated, washed with brine (50 mL×2), dried over Na₂SO₄, filtered and the residue was concentrated to obtain (1-(3,4-difluorobenzyl)imidazo[1,5-a]pyridin-6-yl) methanol (86 mg, 99.18%) which was used in the next step without further purification. LC-MS: m/z 275.2 (M+H)⁺.

### Step E: 1-(3,4-difluorobenzyl)imidazo[1,5-a]pyridine-6-carbaldehyde

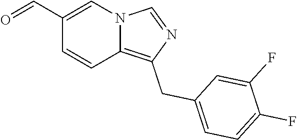 [SMILES: O=Cc1ccc2c(Cc3ccc(F)c(F)c3)ncn2c1]

A mixture of (1-(3,4-difluorobenzyl)imidazo[1,5-a]pyridin-6-yl) methanol (86 mg, 0.314 mmol) and IBX (175.61 mg, 0.627 mmol) in ACN (5 mL) as stirred at 80° C. for 1.5 hrs under N₂ atmosphere. The reaction solution was filtered and dried in vacuo to obtain 1-(3,4-difluorobenzyl)imidazo[1,5-a]pyridine-6-carbaldehyde (85 mg, 99.57%) which was used in the next step without further purification. LC-MS: m/z 273.2 (M+H)⁺.

### Step F: (S)-7-{1-[(3,4-difluorophenyl)methyl]-2,3a-diaza-5-indenyl}-3-[(p-fluorophenyl)methyl]-5-isobutyl-3-methyl-1-oxo-2,4-diaza-6-indancarboxamide (Compound 159)

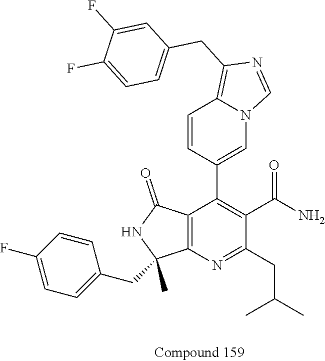 [SMILES: CC(C)Cc1nc2c(c(-c3ccc4c(Cc5ccc(F)c(F)c5)ncn4c3)c1C(N)=O)C(=O)N[C@@]2(C)Cc1ccc(F)cc1]

Compound 159 was then synthesized following the similar route of Example 2, using 1-(3,4-difluorobenzyl)imidazo[1,5-a]pyridine-6-carbaldehyde in step A.

¹H NMR (400 MHz, CD₃OD) δ 8.25 (s, 1H), 7.96 (s, 1H), 7.39 (d, J=9.2 Hz, 1H), 7.03-7.14 (m, 3H), 6.87-6.91 (m, 2H), 6.79-6.84 (m, 2H), 6.47-6.55 (m, 1H), 4.18 (s, 2H), 3.40 (d, J=13.6 Hz, 1H), 3.17 (d, J=13.6 Hz, 1H), 2.85-2.96 (m, 2H), 2.43-2.51 (m, 1H), 1.67 (s, 3H), 1.08 (d, J=6.8 Hz, 3H), 1.02 (d, J=6.8 Hz, 3H). ¹⁹F NMR (377 MHz, CD₃OD) δ −118.29, −141.02, −144.80. LC-MS: m/z 598.3 (M+H)⁺.

### Example 34

### (S)-3-(cyclopropylmethyl)-7-{3-[(5-fluoro-2-pyridyl)methyl]-2-oxo-1,3-benzoxazol-6-yl}-5-isobutyl-3-methyl-1-oxo-2,4-diaza-6-indancarboxamide (Compound 175)

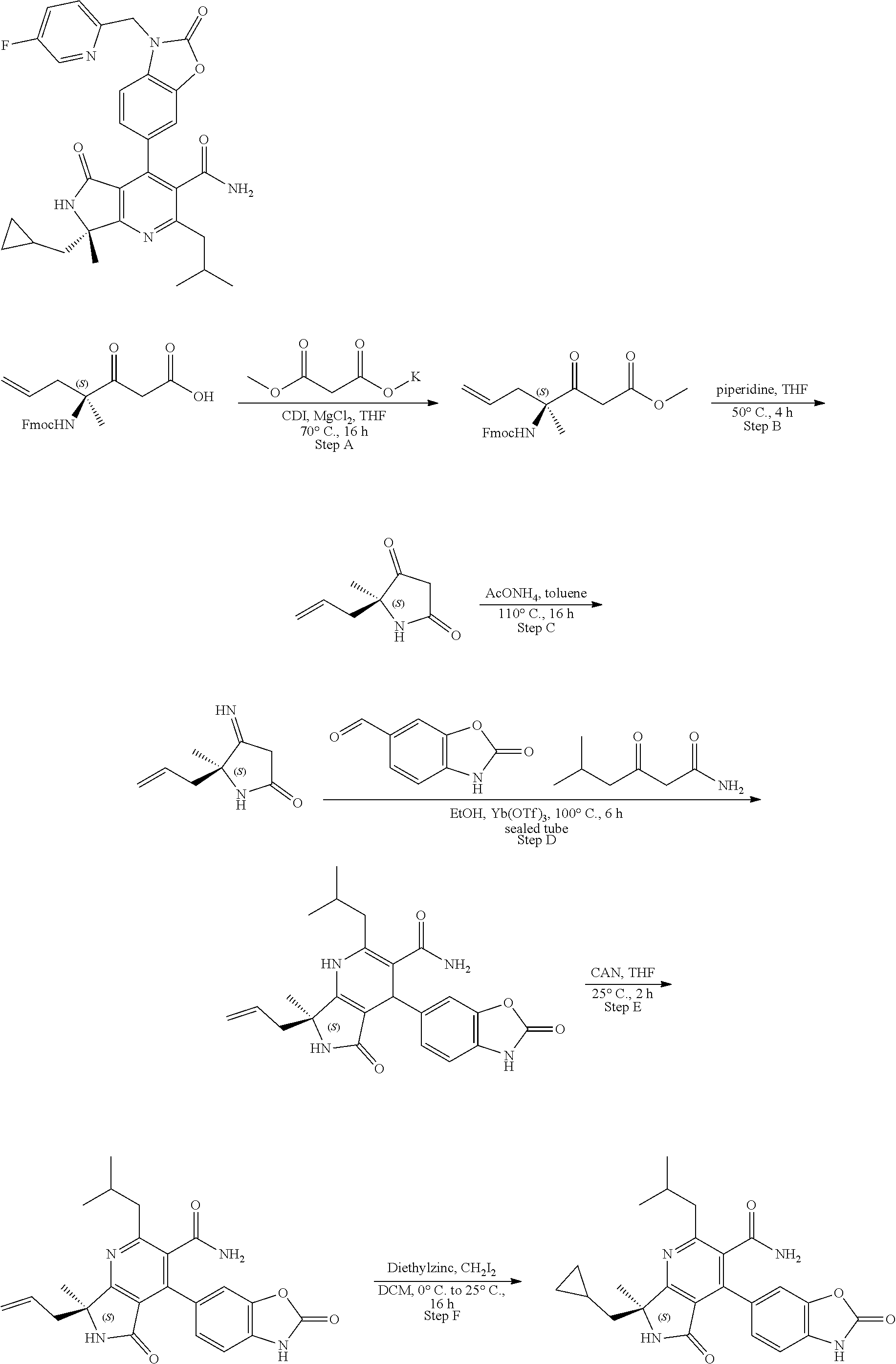

### Step A: methyl(4S)-4-({[(9H-fluoren-9-ylmethyl)oxy]carbonyl}amino)-4-methyl-3-oxohept-6-enoate

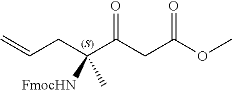

To a solution of CDI (646.02 mg, 3.984 mmol) in THF (10 mL) was added (2S)-2-({[(9H-fluoren-9-ylmethyl)oxy]carbonyl}amino)-2-methylpent-4-enoic acid (700 mg, 1.992 mmol) under Ar. The reaction mixture was stirred at 25° C. for 1 hr. Magnesium chloride (379.28 mg, 3.984 mmol) and [(3-methoxy-1,3-dioxopropyl)oxy]potassium (622.23 mg, 3.984 mmol) were added. The reaction mixture was stirred at 70° C. for 18 hrs under Ar. The reaction mixture was filtered and the filtrate was concentrated under vacuum. The resulting residue was purified by column chromatography (PE/EA=3/2) to afford methyl(4S)-4-({[(9H-fluoren-9-ylmethyl)oxy]carbonyl}amino)-4-methyl-3-oxohept-6-enoate (171 mg, 21.07%) as a yellow solid. LC-MS: m/z 408.3 (M+H)⁺.

### Step B: (5S)-5-methyl-5-(prop-2-enyl)tetrahydropyrrole-2,4-dione

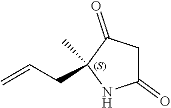

To a solution of methyl(4S)-4-({[(9H-fluoren-9-ylmethyl)oxy]carbonyl}amino)-4-methyl-3-oxohept-6-enoate (171 mg, 0.420 mmol) in THF (10 mL) was added piperidine (1 mL, 10.112 mmol) at 25° C. under Ar. The reaction mixture was stirred at 50° C. for 4 hrs. The reaction mixture was concentrated under vacuum. The resulting residue was purified by column chromatography (DCM/CH₃OH=10/1) to afford (5S)-5-methyl-5-(prop-2-enyl)tetrahydropyrrole-2,4-dione (64 mg, 99.56%) as a yellow solid. LC-MS: m/z 153.8 (M+H)⁺.

### Step C: (5S)-4-azanylidene-5-methyl-5-(prop-2-enyl)tetrahydropyrrol-2-one

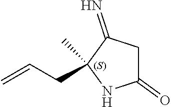

To a solution of (5S)-5-methyl-5-(prop-2-enyl)tetrahydropyrrole-2,4-dione (64 mg, 0.418 mmol) in Toluene (5 mL) was added NH₄OAc (321.71 mg, 4.178 mmol) under N₂. The reaction mixture was stirred at 110° C. for 16 hrs. The reaction mixture was concentrated under vacuum to give (5S)-4-azanylidene-5-methyl-5-(prop-2-enyl)tetrahydropyrrol-2-one (63 mg, 99.07%) as a yellow solid, which was used in the next step without further purification. LC-MS: m/z 153.0 (M+H)⁺.

### Step D: (7S)-7-methyl-2-(2-methylpropyl)-5-oxo-4-(2-oxo-3H-benzo[d][1,3]oxazol-6-yl)-7-(prop-2-enyl)-4,5,6,7-tetrahydro-1H-pyrrolo[4,3-b]pyridine-3-carboxamide

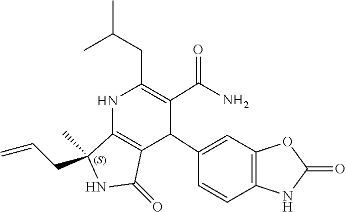

To a solution of 2-oxo-3H-benzo[d][1,3]oxazole-6-carbaldehyde (68 mg, 0.417 mmol) in EtOH (5 mL) was added (5S)-4-azanylidene-5-(cyclopropylmethyl)-5-methyltetrahydropyrrol-2-one (15.62 mg, 0.094 mmol), ytterbium (3+) tris(trifluoromethanesulfonate) (25.85 mg, 0.042 mmol) and 5-methyl-3-oxohexanamide (119.38 mg, 0.834 mmol). The reaction mixture was stirred at 100° C. for 6 hrs in a sealed tube under N₂. The reaction mixture was concentrated under vacuum to afford (7S)-7-methyl-2-(2-methylpropyl)-5-oxo-4-(2-oxo-3H-benzo[d][1,3]oxazol-6-yl)-7-(prop-2-cnyl)-4,5,6,7-tetrahydro-1H-pyrrolo[4,3-b]pyridine-3-carboxamide (70 mg, 39.75%) as a yellow oil, which was used in the next step without further purification. LC-MS: m/z 423.4 (M+H)⁺.

### Step E: (7S)-7-methyl-2-(2-methylpropyl)-5-oxo-4-(2-oxo-3H-benzo[d][1,3]oxazol-6-yl)-7-(prop-2-enyl)-6,7-dihydro-5H-pyrrolo[4,3-b]pyridine-3-carboxamide

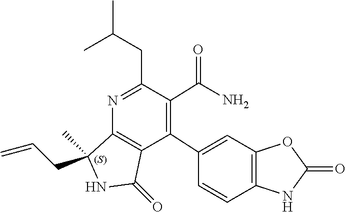

To a solution of (7S)-7-methyl-2-(2-methylpropyl)-5-oxo-4-(2-oxo-3H-benzo[d][1,3]oxazol-6-yl)-7-(prop-2-enyl)-4,5,6,7-tetrahydro-1H-pyrrolo[4,3-b]pyridine-3-carboxamide (70 mg, 0.166 mmol) in THF (5 mL) was added cerium (4+) tetrakis(N-oxonitrite) diammonia bis(dioxo-λ5-azanol) (259.52 mg, 0.473 mmol) under N₂. The reaction mixture was stirred at 25° C. for 2 hrs under N₂. aq. NaHCO₃ was added to the reaction mixture until pH=8. The resulting mixture was poured into H₂O (50 mL) and extracted with EtOAc (50 mL×3). The combined organics were washed with brine (50 mL) and dried over anhydrous Na₂SO₄, filtered and concentrated under vacuum. The resulting residue was purified by prep-TLC (eluting DCM/MeOH=10/1) to afford (7S)-7-methyl-2-(2-methylpropyl)-5-oxo-4-(2-oxo-3H-benzo[d][1,3]oxazol-6-yl)-7-(prop-2-enyl)-6,7-dihydro-5H-pyrrolo[4,3-b]pyridine-3-carboxamide (64 mg, 91.56%) as a white solid. LC-MS: m/z 421.2 (M+H)⁺.

### Step F: (7S)-7-(cyclopropylmethyl)-7-methyl-2-(2-methylpropyl)-5-oxo-4-(2-oxo-3H-benzo[d][1,3]oxazol-6-yl)-6,7-dihydro-5H-pyrrolo[4,3-b]pyridine-3-carboxamide

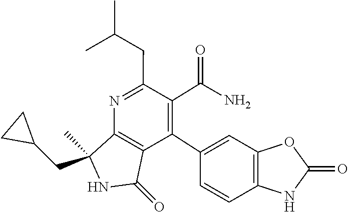 [SMILES: CC(C)Cc1nc2c(c(-c3ccc4[nH]c(=O)oc4c3)c1C(N)=O)C(=O)N[C@@]2(C)CC1CC1]

To a solution of diiodomethane (687.96 mg, 2.569 mmol) in DCM (10 mL) was added diethyl-λ2-zinc(II) (1.284 mL, 2.569 mmol) at 0° C. under Ar, and the reaction mixture was stirred at 0° C. for 2 hrs. (7S)-7-methyl-2-(2-methylpropyl)-5-oxo-4-(2-oxo-3H-benzo[d][1,3]oxazol-6-yl)-7-(prop-2-enyl)-6,7-dihydro-5H-pyrrolo[4,3-b]pyridine-3-carboxamide (54 mg, 0.128 mmol) in DCM was added. The reaction mixture was stirred at 25° C. for 18 hrs. Water (20 mL) was added to quench the reaction. The reaction mixture was extracted with EtOAc (20 mL×3). The combined organic layers were washed with brine (15 mL), dried over anhydrous Na₂SO₄, filtered and concentrated under vacuum. The resulting residue was purified by prep-TLC (PE/EA=1/1) to afford (7S)-7-(cyclopropylmethyl)-7-methyl-2-(2-methylpropyl)-5-oxo-4-(2-oxo-3H-benzo[d][1,3]oxazol-6-yl)-6,7-dihydro-5H-pyrrolo[4,3-b]pyridine-3-carboxamide. LC-MS: m/z 435.4 (M−H)⁺

### (S)-3-(cyclopropylmethyl)-7-{3-[(5-fluoro-2-pyridyl)methyl]-2-oxo-1,3-benzoxazol-6-yl}-5-isobutyl-3-methyl-1-oxo-2,4-diaza-6-indancarboxamide (Compound 175)

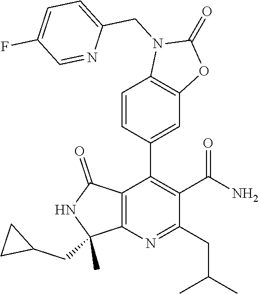 [SMILES: CC(C)Cc1nc2c(c(-c3ccc4c(c3)oc(=O)n4Cc3ccc(F)cn3)c1C(N)=O)C(=O)N[C@@]2(C)CC1CC1]

Compound 175 was then synthesized following the similar route of Example 26, using (7S)-7-(cyclopropylmethyl)-7-methyl-2-(2-methylpropyl)-5-oxo-4-(2-oxo-3H-benzo[d][1,3]oxazol-6-yl)-6,7-dihydro-5H-pyrrolo[4,3-b]pyridine-3-carboxamide and 2-(bromomethyl)-5-fluoropyridine in step D. ¹H NMR (400 MHz, DMSO-d₆) δ 8.69 (s, 1H), 8.54 (d, J=2.8 Hz, 1H), 7.76-7.81 (m, 2H), 7.65 (dd, J=8.4, 4.4 Hz, 1H), 7.45 (s, 1H), 7.35 (s, 1H), 7.18 (s, 2H), 5.21 (s, 2H), 2.73 (d, J=7.2 Hz, 2H), 2.23-2.30 (m, 1H), 1.77 (d, J=6.8 Hz, 2H), 1.44 (s, 3H), 0.94 (d, J=6.8 Hz, 3H), 0.88 (d, J=6.4 Hz, 3H), 0.40-0.43 (m, 1H), 0.29-0.33 (m, 1H), 0.05-0.15 (m, 2H), −0.30-−0.27 (m, 1H). ¹⁹F NMR (377 MHz, DMSO-d₆) δ −128.71, LC-MS: m/z 544.6 (M+H)⁺.

### Example 35

### (S)-3-[(p-fluorophenyl)methyl]-7-{1-[(5-fluoro-2-pyridyl)methyl]-2-(trifluoromethyl)-1H-1,3-benzimidazol-5-yl}-5-isobutyl-3-methyl-1-oxo-2,4-diaza-6-indancarboxamide (Compound 170)

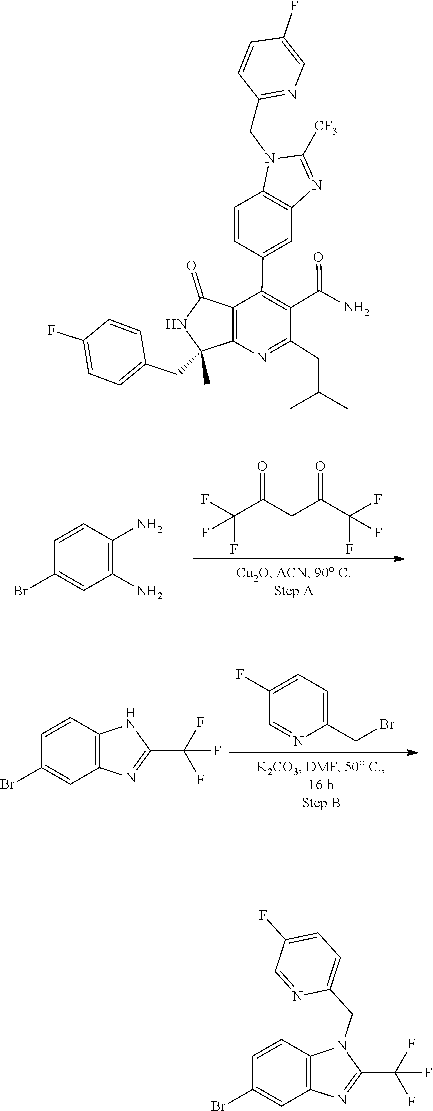 [SMILES: CC(C)Cc1nc2c(c(-c3ccc4c(c3)nc(C(F)(F)F)n4Cc3ccc(F)cn3)c1C(N)=O)C(=O)N[C@@]2(C)Cc1ccc(F)cc1 ; Nc1ccc(Br)cc1N ; O=C(CC(=O)C(F)(F)F)C(F)(F)F ; FC(F)(F)c1nc2cc(Br)ccc2[nH]1 ; Fc1ccc(CBr)nc1 ; Fc1ccc(Cn2c(C(F)(F)F)nc3cc(Br)ccc32)nc1]

### Step A: 5-bromo-2-(trifluoromethyl)-1H-benzo[d]imidazole

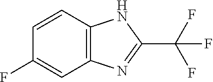 [SMILES: Fc1ccc2[nH]c(C(F)(F)F)nc2c1]

To a solution of 4-bromobenzene-1,2-diamine (2 g, 10.693 mmol) in ACN (20 mL) were added 1,1,1,5,5,5-hexafluoropentane-2,4-dione (2.67 g, 12.831 mmol) and Cu₂O (310 mg, 2.139 mmol) at 25° C., the reaction mixture was stirred at 90° C. for 16 hrs. The mixture was concentrated under vacuum. The resulting residue was purified by prep-TLC (eluting PE/EA=5/1) to afford 5-bromo-2-(trifluoromethyl)-1H-benzo[d]imidazole (2.10 g, 74.10%) as a yellow solid. LC-MS: m/z 266.6 (M+H)⁺.

### Step B: 5-bromo-1-((5-fluoropyridin-2-yl)methyl)-2-(trifluoromethyl)-1H-benzo[d]imidazole

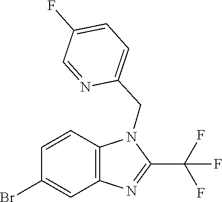 [SMILES: Fc1ccc(Cn2c(C(F)(F)F)nc3cc(Br)ccc32)nc1]

To a solution of 5-bromo-2-(trifluoromethyl)-1H-benzo[d]imidazole (400 mg, 1.509 mmol) in DMF (50 mL) were added 2-(bromomethyl)-5-fluoropyridine (573.58 mg, 3.019 mmol) and potassium carbonate (1042.90 mg, 7.546 mmol) at 25° C. under N₂. The reaction mixture was stirred at 50° C. for 16 hrs. The reaction mixture was quenched by NH₄Cl (10 mL), the mixture was extracted with EA (30 mL), washed with brine (30 mL), dried over sodium sulfate, filtered and concentrated under vacuum. The resulting residue was purified by TLC (PE/EA=4/1) to afford 5-bromo-1-((5-fluoropyridin-2-yl)methyl)-2-(trifluoromethyl)-1H-benzo[d]imidazole (217 mg, 38.43%) as a colorless oil. ¹H NMR (400 MHz, CDCl₃) δ 8.42 (d, J=4.8 Hz, 1H), 8.03 (d, J=1.6 Hz, 1H), 7.47-7.50 (m, 1H), 7.33-7.37 (m, 2H), 6.95-6.98 (m, 1H), 5.58 (d, J=9.2 Hz, 2H).

### (S)-3-[(p-fluorophenyl)methyl]-7-{1-[(5-fluoro-2-pyridyl)methyl]-2-(trifluoromethyl)-1H-1,3-benzimidazol-5-yl}-5-isobutyl-3-methyl-1-oxo-2,4-diaza-6-indancarboxamide (Compound 170)

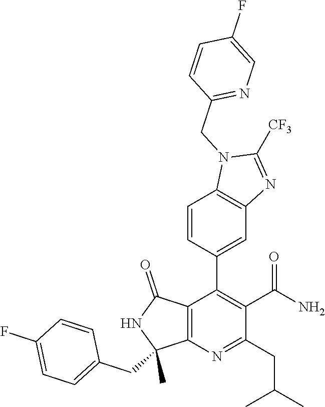 [SMILES: CC(C)Cc1nc2c(c(-c3ccc4c(c3)nc(C(F)(F)F)n4Cc3ccc(F)cn3)c1C(N)=O)C(=O)N[C@@]2(C)Cc1ccc(F)cc1]

Compound 170 was then synthesized following the similar route of Example 25, using 5-bromo-1-((5-fluoropyridin-2-yl)methyl)-2-(trifluoromethyl)-1H-benzo[d]imidazole in step B.

¹H NMR (400 MHz, DMSO-d₆) δ 8.69 (s, 1H), 8.46 (d, J=2.8 Hz, 1H), 7.76-7.81 (m, 1H), 7.73 (s, 1H), 7.54-7.65 (m, 3H), 7.40 (s, 1H), 7.04-7.14 (m, 1H), 6.92 (t, J=8.8 Hz, 2H), 7.82-7.86 (m, 2H), 5.80 (s, 2H), 3.25 (d, J=13.6 Hz, 1H), 3.06 (d, J=13.6 Hz, 1H), 2.73-2.82 (m, 2H), 2.37-2.44 (m, 1H), 1.57 (s, 3H), 1.03 (d, J=6.8 Hz, 3H), 0.93 (d, J=6.8 Hz, 3H). ¹⁹F NMR (377 MHz, DMSO-d₆) δ −60.82, −116.67, −128.62. LC-MS: m/z 649.5 (M+H)⁺.

### Example 36

### (S)-7-{1-[(5-cyano-2-pyridyl)methyl]-2-methyl-1H-1,7-diazainden-5-yl}-3-[(p-fluorophenyl)methyl]-5-isobutyl-3-methyl-1-oxo-2,4-diaza-6-indancarboxamide (Compound 169)

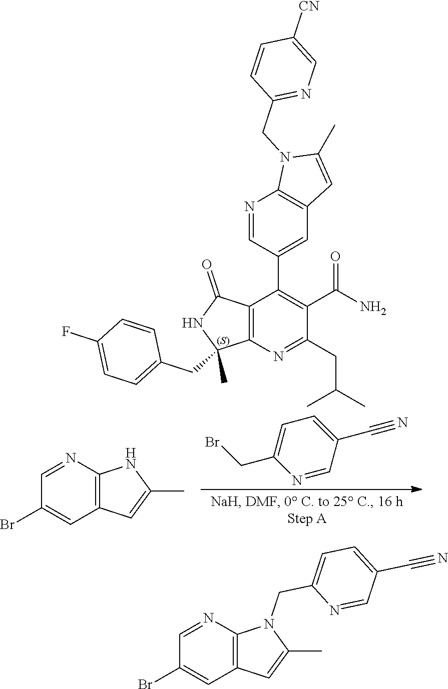

### Step A: 6-((5-bromo-2-methyl-1H-pyrrolo[2,3-b]pyridin-1-yl)methyl) nicotinonitrile

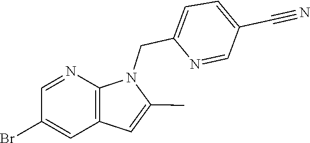 [SMILES: Cc1cc2cc(Br)cnc2n1Cc1ccc(C#N)cn1]

To a solution of 5-bromo-2-methyl-1H-pyrrolo[2,3-b]pyridine (228.52 mg, 1.083 mmol) in DMF (10 mL) was added NaH (51.97 mg, 2.165 mmol) at 0° C. under Ar. The reaction mixture was stirred at 0° C. for 0.5 hr under Ar. 6-(bromomethyl) nicotinonitrile (640.0 mg, 3.248 mmol) was added and the reaction mixture was stirred at rt for 16 hrs under Ar. The reaction mixture was poured into H₂O (50 mL) and extracted with EA (50 mL×2). The combined organics were washed with brine (20 mL) and dried over sodium sulfate, filtered and concentrated under vacuum. The resulting residue was purified by silica gel column chromatography (eluting PE/EA=20/1) to afford 6-((5-bromo-2-methyl-1H-pyrrolo[2,3-b]pyridin-1-yl)methyl) nicotinonitrile (290 mg, 81.86%) as a white solid. LC-MS: m/z 329.1 (M+H)⁺.

### (S)-7-{1-[(5-cyano-2-pyridyl)methyl]-2-methyl-1H-1,7-diazainden-5-yl}-3-[(p-fluorophenyl)methyl]-5-isobutyl-3-methyl-1-oxo-2,4-diaza-6-indancarboxamide (Compound 169)

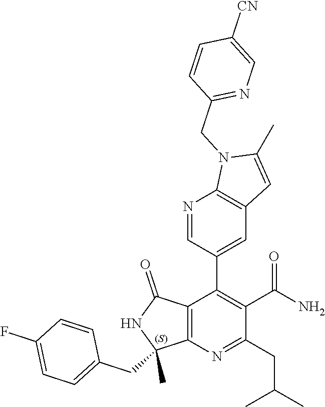

Compound 169 was then synthesized following the similar route of Example 25, using 6-((5-bromo-2-methyl-1H-pyrrolo[2,3-b]pyridin-1-yl)methyl)nicotinonitrile in step B.

¹H NMR (400 MHz, DMSO-d₆) δ 8.94 (d, J=1.6 Hz, 1H), 8.68 (s, 1H), 8.26 (dd, J=8.0, 2.0 Hz, 1H), 7.62-7.79 (m, 3H), 7.41 (s, 1H), 7.30 (d, J=8.4 Hz, 1H), 6.89-6.93 (m, 2H), 6.82-6.86 (m, 2H), 6.31 (d, J=0.8 Hz, 1H), 5.66 (s, 2H), 3.25 (d, J=13.6 Hz, 1H), 3.06 (d, J=13.6 Hz, 1H), 2.74-2.83 (m, 2H), 2.35 (s, 3H), 2.31-2.33 (m, 1H), 1.56 (s, 3H), 1.03 (d, J=6.8 Hz, 3H), 0.93 (d, J=6.4 Hz, 3H). ¹⁹F NMR (377 MHz, DMSO-d₆) δ −116.67. LC-MS: m/z 602.4 (M+H)⁺.

### Example 37

### (S)-7-{3-[(3,4-difluorophenyl)methyl]-1,2-benzisoxazol-6-yl}-3-[(p-fluorophenyl)methyl]-5-isobutyl-3-methyl-1-oxo-2,4-diaza-6-indancarboxamide (Compound 174)

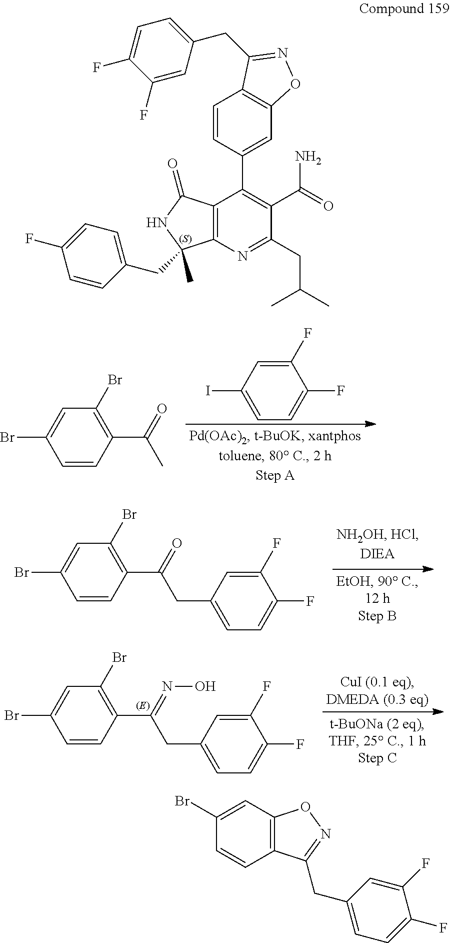

### Step A: 1-(2,4-dibromophenyl)-2-(3,4-difluorophenyl) ethan-1-one

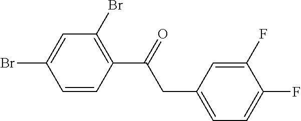 [SMILES: O=C(Cc1ccc(F)c(F)c1)c1ccc(Br)cc1Br]

To a solution of 1-(2,4-dibromophenyl) ethan-1-one (828.06 mg, 2.979 mmol) in Toluene (20 mL) and 1,2-difluoro-4-iodobenzene (650 mg, 2.708 mmol) were added Potassium tert-butoxide (395.09 mg, 3.521 mmol), PALLADIUM ACETATE (60.81 mg, 0.271 mmol) and XANT PHOS (313.44 mg, 0.542 mmol). The reaction mixture was stirred at 90° C. for 2 hrs under N₂. The mixture was concentrated in vacuum, the residue was purified by flash column chromatography (eluting PE/EA=25/1) to afford 1-(2,4-dibromophenyl)-2-(3,4-difluorophenyl) ethan-1-one (647 mg, 61.25%) as a yellow oil. ¹H NMR (400 MHz, CDCl₃) δ 7.68 (s, 1H), 7.28 (dd, J=8.4, 1.2 Hz, 1H), 7.16 (d, J=8.4 Hz, 1H), 7.00-7.06 (m, 2H), 6.96-6.97 (m, 1H), 4.20 (s, 2H).

### Step B: (E)-1-(2,4-dibromophenyl)-2-(3,4-difluorophenyl)ethan-1-one oxime

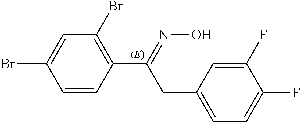 [SMILES: O/N=C(\Cc1ccc(F)c(F)c1)c1ccc(Br)cc1Br]

To a solution of 1-(2,4-dibromophenyl)-2-(3,4-difluorophenyl) ethan-1-one (647 mg, 1.659 mmol) in EtOH (20 mL) were added azanol hydrochloride (576.38 mg, 8.294 mmol) and DIEA (1072.06 mg, 8.294 mmol). The reaction mixture was stirred at 90° C. for 16 hrs under N₂. The mixture was washed with brine (20 mL), dried over sodium sulfate, filtered and concentrated in vacuum to afford (E)-1-(2,4-dibromophenyl)-2-(3,4-difluorophenyl) ethan-1-one oxime (500 mg, 74.41%) as a yellow solid, which was used in the next step without further purification. LC-MS: m/z 403.8 (M+H)⁺.

### Step C: 6-bromo-3-(3,4-difluorobenzyl)benzo[d]isoxazole

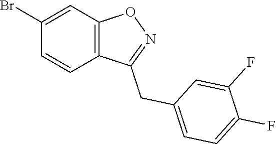 [SMILES: Fc1ccc(Cc2noc3cc(Br)ccc23)cc1F]

To a solution of (E)-1-(2,4-dibromophenyl)-2-(3,4-difluorophenyl)ethan-1-one oxime (430 mg, 1.062 mmol) in 2,5-diazahexane (28.07 mg, 0.318 mmol) and THF (30 mL) were added CuI (20.22 mg, 0.106 mmol) and sodium 2-methylpropan-2-olate (204.04 mg, 2.123 mmol). The reaction mixture was stirred at 25° C. for 1 hr under N₂. The reaction mixture was concentrated in vacuum, the residue was purified by flash column chromatography (eluting PE/EA=10/1) to afford 6-bromo-3-(3,4-difluorobenzyl)benzo[d]isoxazole (170 mg, 49.41%) as a yellow oil. ¹H NMR (400 MHz, CDCl₃) δ 7.68 (s, 1H), 7.28 (dd, J=8.4, 1.2 Hz, 1H), 7.16 (d, J=8.4 Hz, 1H), 7.00-7.06 (m, 2H), 6.96-6.97 (m, 1H), 4.20 (s, 2H).

### (S)-7-{3-[(3,4-difluorophenyl)methyl]-1,2-benzisoxazol-6-yl}-3-[(p-fluorophenyl)methyl]-5-isobutyl-3-methyl-1-oxo-2,4-diaza-6-indancarboxamide (Compound 174)

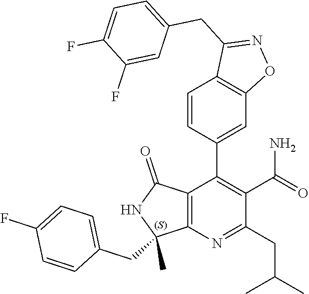

Compound 174 was then synthesized following the similar route of Example 25, using 6-bromo-3-(3,4-difluorobenzyl)benzo[d]isoxazole in step B.

¹H NMR (400 MHz, DMSO-d₆) δ 8.73 (s, 1H), 7.69-7.71 (m, 2H), 7.50-7.55 (m, 1H), 7.35-7.42 (m, 3H), 7.26-7.27 (m, 1H), 7.02 (m, 1H), 6.92 (t, J=8.8 Hz, 2H), 6.82-6.85 (m, 2H), 4.40 (s, 2H), 3.25 (d, J=13.6 Hz, 1H), 3.06 (d, J=13.2 Hz, 1H), 2.72-2.80 (m, 2H), 2.35-2.39 (m, 1H), 1.56 (s, 3H), 1.02 (d, J=6.8 Hz, 3H), 0.92 (d, J=6.8 Hz, 3H). ¹⁹F NMR (377 MHz, DMSO-d₆) δ −116.64, −138.31, −141.15. LC-MS: m/z 599.6 (M+H)⁺.

### Example 38

### (S)-3-[(p-fluorophenyl)methyl]-7-{4-[(5-fluoro-2-pyridyl)methyl]-3-oxo-2,4-dihydro-1,4-benzoxazin-7-yl}-5-isobutyl-3-methyl-1-oxo-2,4-diaza-6-indancarboxamide (Compound 173)

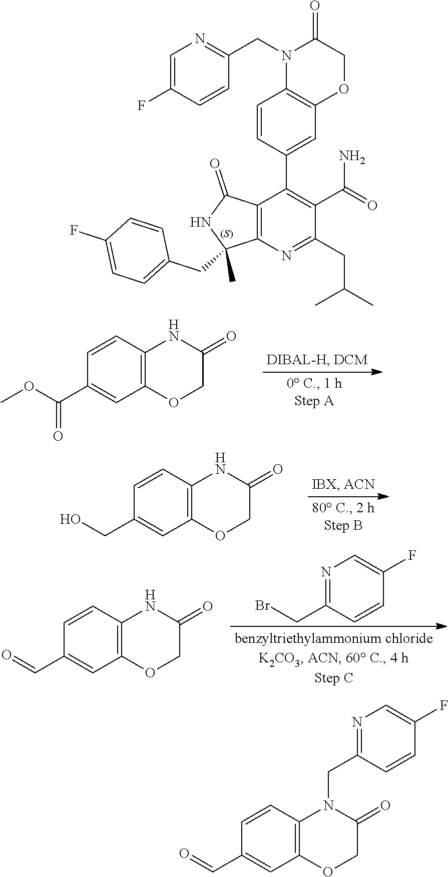

### Step A: 7-(hydroxymethyl)-2H-benzo[b][1,4]oxazin-3(4H)-one

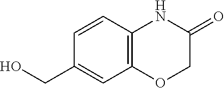 [SMILES: O=C1COc2cc(CO)ccc2N1]

To a solution of methyl 3-oxo-3,4-dihydro-2H-benzo[b][1,4]oxazine-7-carboxylate (2 g, 9.652 mmol) in DCM (20 mL) was added DIBAL-H (4.12 g, 28.96 mmol) at 0° C., the reaction mixture was stirred at 0° C. for 1 hr under N₂. The mixture was added to water (30 mL), extracted with DCM (30 mL×2), the combined organic layers were washed with brine (30 mL×3), dried over sodium sulfate, filtered and concentrated under vacuum to afford 7-(hydroxymethyl)-2H-benzo[b][1,4]oxazin-3(4H)-one (1.4 g, 80.9%) as a yellow oil. LC-MS: m/z 180.2 (M+H)⁺.

### Step B: 3-oxo-3,4-dihydro-2H-benzo[b][1,4]oxazine-7-carbaldehyde

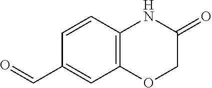 [SMILES: O=Cc1ccc2c(c1)OCC(=O)N2]

A mixture of 7-(hydroxymethyl)-2H-benzo[b][1,4]oxazin-3(4H)-one (1.4 g, 7.813 mmol) and IBX (4.38 g, 15.627 mmol) in ACN (20 mL) as stirred at 80° C. for 2 hr under N₂. The reaction solution was filtered and purified by prep-HPLC (50% of CH₃CN in water, 0.1% FA) to give 3-oxo-3,4-dihydro-2H-benzo[b][1,4]oxazine-7-carbaldehyde (130 mg, 9.39%) as a yellow oil. LC-MS: m/z 178.0 (M+H)⁺.

### Step C: 4-((5-fluoropyridin-2-yl)methyl)-3-oxo-3,4-dihydro-2H-benzo[b][1,4]oxazine-7-carbaldehyde

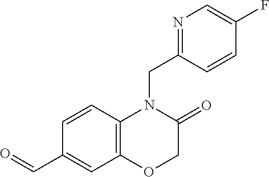 [SMILES: O=Cc1ccc2c(c1)OCC(=O)N2Cc1ccc(F)cn1]

To a solution of 3-oxo-3,4-dihydro-2H-benzo[b][1,4]oxazine-7-carbaldehyde (130 mg, 0.734 mmol) in ACN (10 mL) were added 2-(bromomethyl)-5-fluoropyridine (139.44 mg, 0.734 mmol), Benzyltriethylammonium chloride (167.14 mg, 0.734 mmol) and K₂CO₃ (253.53 mg, 1.834 mmol), the mixture was stirred at 60° C. for 16 hrs under N₂. The reaction mixture was poured into H₂O (50 mL) and extracted with EA (50 mL×2). The combined organics were washed brine (20 mL) and dried over sodium sulfate, filtered and concentrated under vacuum. The resulting residue was purified by silica gel column chromatography (eluting PE/EA=10/1) to afford 4-((5-fluoropyridin-2-yl)methyl)-3-oxo-3,4-dihydro-2H-benzo[b][1,4]oxazine-7-carbaldehyde (70 mg, 33.32%) as a yellow oil. LC-MS: m/z 287.0 (M+H)⁺.

### (S)-3-[(p-fluorophenyl)methyl]-7-{4-[(5-fluoro-2-pyridyl)methyl]-3-oxo-2,4-dihydro-1,4-benzoxazin-7-yl}-5-isobutyl-3-methyl-1-oxo-2,4-diaza-6-indancarboxamide (Compound 173)

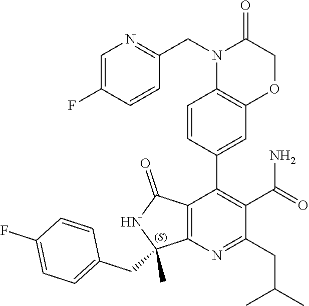

Compound 173 was then synthesized following the similar route of Example 2, using 4-((5-fluoropyridin-2-yl)methyl)-3-oxo-3,4-dihydro-2H-benzo[b][1,4]oxazine-7-carbaldehyde in step B.

¹H NMR (400 MHz, DMSO-d₆) δ 8.66 (s, 1H), 8.53 (d, J=2.4 Hz, 1H), 7.71-7.74 (m, 2H), 7.45-7.50 (m, 2H), 6.87-6.95 (m, 3H), 6.79-6.83 (m, 2H), 6.75 (s, 1H), 6.70 (d, J=8.0 Hz, 1H), 5.19 (s, 2H), 4.76 (s, 2H), 3.23 (d, J=13.6 Hz, 1H), 3.04 (d, J=13.6 Hz, 1H), 2.73-2.78 (m, 2H), 2.34-2.37 (m, 1H), 1.54 (s, 3H), 1.01 (d, J=6.8 Hz, 3H), 0.91 (d, J=6.4 Hz, 3H). ¹⁹F NMR (377 MHz, DMSO-d₆) δ −116.67, −129.44. LC-MS: m/z 612.3 (M+H)⁺.

### 4-{3-[(5-cyano-2-pyridyl)methyl]-2-oxo-1,3-benzoxazol-6-yl}-3-ethyl-1-[(p-fluorophenyl)methyl]-6-isobutyl-1H-1,2,7-triazaindene-5-carboxamide (Compound 177)

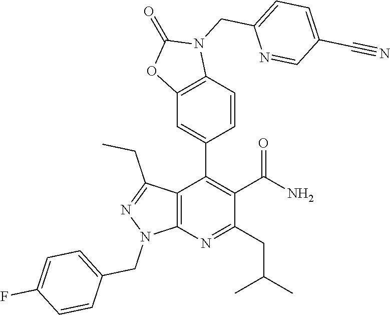 [SMILES: CCc1nn(Cc2ccc(F)cc2)c2nc(CC(C)C)c(C(N)=O)c(-c3ccc4c(c3)oc(=O)n4Cc3ccc(C#N)cn3)c12]

Compound 177 was synthesized following the similar route of Example 18, using 3-oxopentanenitrile in step C and then following the similar route of Example 1, by using 6-[(6-formyl-2-oxo-1,3-benzoxazol-3-yl)methyl]pyridine-3-carbonitrile and 5-ethyl-2-[(4-fluorophenyl)methyl]pyrazol-3-amine in step D.

¹H NMR (400 MHz, CD₃OD) δ 8.85 (d, J=1.6 Hz, 1H), 8.20 (dd, J=8.0, 2.0 Hz, 1H), 7.67 (d, J=8.0 Hz, 1H), 7.42 (d, J=0.8 Hz, 1H), 7.37 (s, 2H), 7.23-7.27 (m, 1H), 7.16-7.21 (m, 1H), 7.00 (t, J=8.4 Hz, 2H), 5.63 (s, 2H), 5.32 (d, J=3.2 Hz, 2H), 2.89 (d, J=7.6 Hz, 2H), 2.42-2.49 (m, 1H), 2.34-2.39 (m, 2H), 1.00 (d, J=6.4 Hz, 6H), 0.87 (t, J=7.6 Hz, 3H). ¹⁹F NMR (377 MHz, CD₃OD) δ −117.0. LC-MS: m/z 604.1 (M+H)⁺.

### Example 43

### 4-{3-[(5-cyano-2-pyridyl)methyl]-2-oxo-1,3-benzoxazol-6-yl}-1-[(p-fluorophenyl)methyl]-6-isobutyl-1H-1,2,7-triazaindene-5-carboxamide (Compound 176)

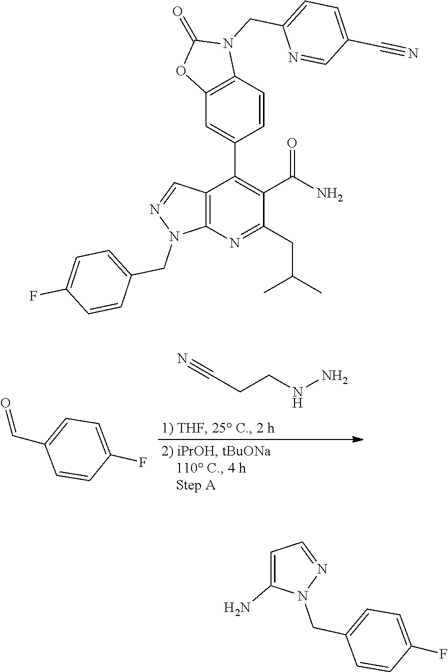

### Step A: 1-(4-fluorobenzyl)-1H-pyrazol-5-amine

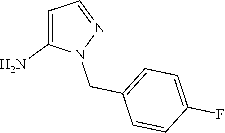 [SMILES: Nc1ccnn1Cc1ccc(F)cc1]

To a solution of 4-fluorobenzaldehyde (2.32 g, 18.68 mmol) in THF (10 mL) was added 3-hydrazinopropanenitrile (1.5 g, 17.62 mmol) dropwisely. The mixture was stirred at 25° C. for 2 hrs. Then the mixture was concentrated to give a residue, then to the residue were added i-PrOH (15 mL) and NaOtBu (1.69 g, 17.62 mmol), the mixture was stirred at 110° C. for another 4 hrs. The reaction mixture was quenched by addition water (20 mL) at 20° C. extracted with EA (25 mL*3). The combined organic layers were washed with brine (30 mL*2), dried over anhydrous Na₂SO₄, filtered and concentrated under reduced pressure to give a residue. The residue was purified by flash silica gel chromatography to give 1-(4-fluorobenzyl)-1H-pyrazol-5-amine (2.5 g, 74.19%) as a colorless oil. LC-MS: m/z 192.1 (M+H)⁺.

### 4-{3-[(5-cyano-2-pyridyl)methyl]-2-oxo-1,3-benzoxazol-6-yl}-1-[(p-fluorophenyl)methyl]-6-isobutyl-1H-1,2,7-triazaindene-5-carboxamide (Compound 176)

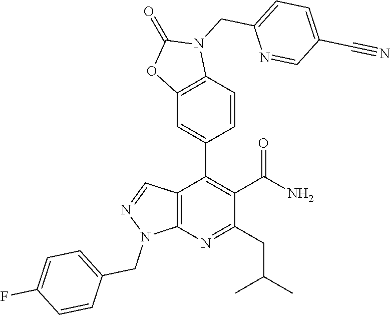 [SMILES: CC(C)Cc1nc2c(cnn2Cc2ccc(F)cc2)c(-c2ccc3c(c2)oc(=O)n3Cc2ccc(C#N)cn2)c1C(N)=O]

Compound 176 was then synthesized following the similar route of Example 30, using 1-(4-fluorobenzyl)-1H-pyrazol-5-amine and 6-[(6-formyl-2-oxo-1,3-benzoxazol-3-yl)methyl]pyridine-3-carbonitrile in step D.

¹H NMR (400 MHz, CDCl₃) δ 8.88 (d, J=1.2 Hz, 1H), 8.02 (dd, J=8.0, 2.0 Hz, 1H), 7.83 (s, 1H), 7.51-7.59 (m, 2H), 7.35-7.47 (m, 3H), 7.14 (d, J=8.0 Hz, 1H), 6.99 (t, J=8.8 Hz, 2H), 5.69 (s, 2H), 5.59 (d, J=2.0 Hz, 1H), 5.36 (br s, 1H), 5.22 (s, 2H), 2.95 (d, J=7.2 Hz, 2H), 2.43-2.51 (m, 1H), 1.02 (d, J=6.8 Hz, 6H). ¹⁹F NMR (377 MHz, CDCl₃) δ −114.4. LC-MS: m/z 576.0 (M+H)⁺.

### Example 44

### 3-cyano-4-{3-[(5-cyano-2-pyridyl)methyl]-2-oxo-1,3-benzoxazol-6-yl}-1-[(p-fluorophenyl)methyl]-6-isobutyl-1H-1,7-diazaindene-5-carboxamide (Compound 166)

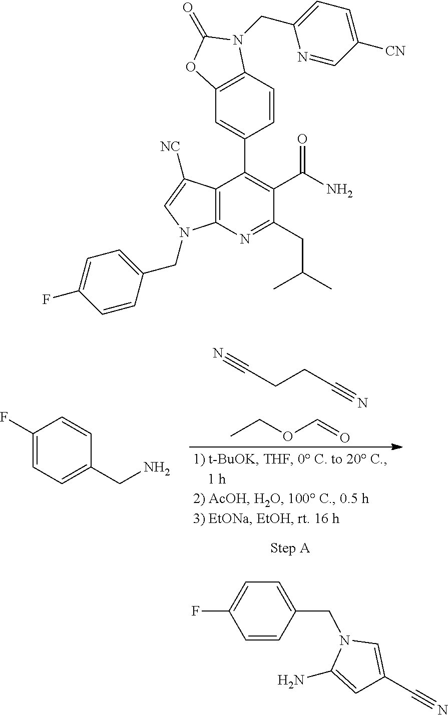

### Step A: 5-amino-1-(4-fluorobenzyl)-1H-pyrrole-3-carbonitrile

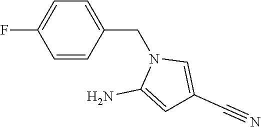 [SMILES: N#Cc1cc(N)n(Cc2ccc(F)cc2)c1]

To a solution of potassium 2-methylpropan-2-olate (6.73 g, 59.933 mmol) in THF (100 mL) was added a solution of ethyl methanoate (4 mL, 49.730 mmol) and butanedinitrile (4.053 mL, 49.944 mmol) in THF (20 mL) at 0° C. under N₂, the mixture was stirred at 25° C. for 1 hr under N₂. The mixture was concentrated under vacuum. The residue was dissolved in H₂O (20 mL), (4-fluorophenyl) methanamine (6.25 g, 49.944 mmol) and AcOH (20 mL) were added and the reaction mixture was stirred at 100° C. for another 30 min. The mixture was concentrated under vacuum. The resulting residue was added to water (100 mL), extracted with EA (100 mL×2), the combined organic layers were washed with aq. NaOH (2 N, 80 mL×2) and brine (100 mL), dried over sodium sulfate, filtered and concentrated under vacuum. The residue was dissolved in EtOH (50 mL), sodium ethanolate (6.8 g, 99.927 mmol) was added and the reaction mixture was stirred at 25° C. for 16 hrs under N₂. The mixture was added to ice-water (200 mL), extracted with EA (100 mL×3), the combined organic layers were washed with brine (100 mL), dried over sodium sulfate, filtered and concentrated under vacuum, the resulting residue was purified by silica gel chromatography (eluting PE/EA=3/1) to afford 5-amino-1-(4-fluorobenzyl)-1H-pyrrole-3-carbonitrile (5.27 g, 49.03%) as a brown oil. ¹H NMR (400 MHz, DMSO-d₆) δ 7.19-7.30 (m, 5H), 5.45 (d, J=2.4 Hz, 1H), 5.06 (s, 2H), 4.89 (s, 2H). LC-MS: m/z 216.3 (M+H)⁺.

### 3-cyano-4-{3-[(5-cyano-2-pyridyl)methyl]-2-oxo-1,3-benzoxazol-6-yl}-1-[(p-fluorophenyl)methyl]-6-isobutyl-1H-1,7-diazaindene-5-carboxamide (Compound 166)

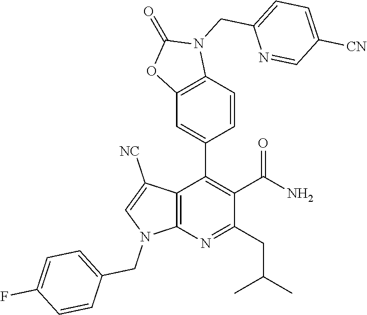 [SMILES: CC(C)Cc1nc2c(c(C#N)cn2Cc2ccc(F)cc2)c(-c2ccc3c(c2)oc(=O)n3Cc2ccc(C#N)cn2)c1C(N)=O]

Compound 166 was then synthesized following the similar route of Example 36, using 5-amino-1-(4-fluorobenzyl)-1H-pyrrole-3-carbonitrile in step A.

¹H NMR (400 MHz, CD₃OD) δ 8.84-8.85 (m, 1H), 8.26 (s, 1H), 8.16 (dd, J=8.4, 2.4 Hz, 1H), 7.61 (d, J=8.4 Hz, 1H), 7.40-7.43 (m, 3H), 7.28 (dd, J=8.0, 1.6 Hz, 1H), 7.14 (d, J=8 Hz, 1H), 7.01-7.06 (m, 2H), 5.54 (s, 2H), 5.30 (d, J=2.4 Hz, 2H), 2.88 (d, J=7.2 Hz, 2H), 2.40-2.45 (m, 1H), 0.97 (d, J=6.8 Hz, 6H). ¹⁹F NMR (377 MHz, CD₃OD) δ −116.25. LC-MS: m/z 600.3 (M+H)⁺.

### Example 45

### 3-[(p-fluorophenyl)methyl]-7-{3-[(5-fluoro-2-pyridyl)methyl]-2-oxo-1,3-benzoxazol-6-yl}-5-isobutyl-3H-1,3,4-triazaindene-6-carboxamide (Compound 171)

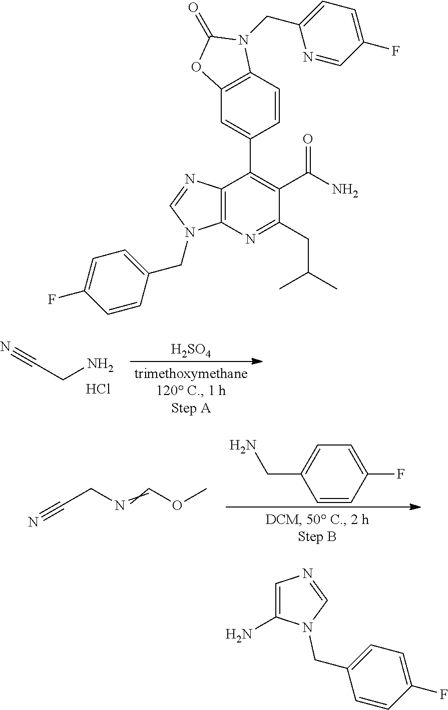

### Step A: methyl N-(cyanomethyl)formimidate

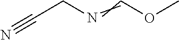

To a solution of trimethoxymethane (15 mL) was added H₂SO₄ (17.49 mg, 178.36 μmol). The mixture was stirred at 120° C. for 0.5 hr. Then 2-aminoacetonitrile (1 g, 17.84 mmol) in trimethoxymethane (3 mL) was added and the mixture was stirred for another 0.5 hr. The mixture was filtered and then concentrated to give methyl N-(cyanomethyl)formimidate (1.2 g, crude) as a brown oil, which was used in the next step without further purification. LC-MS: m/z 216.3 (M+H)⁺.

### Step B: 1-(4-fluorobenzyl)-1H-imidazol-5-amine

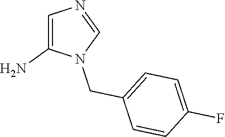 [SMILES: Nc1cncn1Cc1ccc(F)cc1]

To a solution of methyl N-(cyanomethyl)formimidate (500 mg, 5.10 mmol) in DCM (5 mL) was added (4-fluorophenyl) methanamine (709.30 mg, 5.10 mmol). The mixture was stirred at 50° C. for 2 hrs. The mixture was concentrated to give a residue. The crude product was purified by prep-HPLC (column: C18 150×30 mm; mobile phase: [water (NH₄HCO₃)-ACN]; gradient: 25%-55% B over 7 min) to give 1-(4-fluorobenzyl)-1H-imidazol-5-amine (200 mg, 19.12%) as a brown solids. LC-MS: m/z 192.1 (M+H)⁺.

### 3-[(p-fluorophenyl)methyl]-7-{3-[(5-fluoro-2-pyridyl)methyl]-2-oxo-1,3-benzoxazol-6-yl}-5-isobutyl-3H-1,3,4-triazaindene-6-carboxamide (Compound 171)

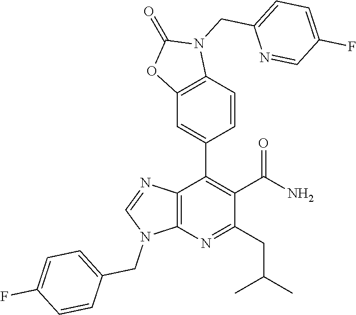 [SMILES: CC(C)Cc1nc2c(ncn2Cc2ccc(F)cc2)c(-c2ccc3c(c2)oc(=O)n3Cc2ccc(F)cn2)c1C(N)=O]

Compound 171 was then synthesized following the similar route of Example 1, using 1-(4-fluorobenzyl)-1H-imidazol-5-amine and 3-[(5-fluoro-2-pyridyl)methyl]-2-oxo-1,3-benzoxazole-6-carbaldehyde in step D.

¹H NMR (400 MHz, CD₃OD) § 8.43 (s, 1H) 8.41 (d, J=2.8 Hz, 1H) 7.60-7.66 (m, 1H) 7.52-7.56 (m, 2H) 7.43-7.49 (m, 3H) 7.17 (d, J=8.0 Hz, 1H) 7.03-7.08 (m, 2H) 5.54 (s, 2H) 5.21 (s, 2H) 2.89 (d, J=7.6 Hz, 2H) 2.33-2.46 (m, 1H) 0.97 (d, J=6.8 Hz, 6H). ¹⁹F NMR (377 MHz, CD₃OD) δ −116.24, −130.12. LC-MS: m/z 569.1 (M+H)⁺.

### Example 46

### 4-{3-[(5-cyano-2-pyridyl)methyl]-2-oxo-1,3-benzoxazol-6-yl}-6-isobutyl-1-isopropyl-1H-1,2,7-triazaindene-5-carboxamide (Compound 178)

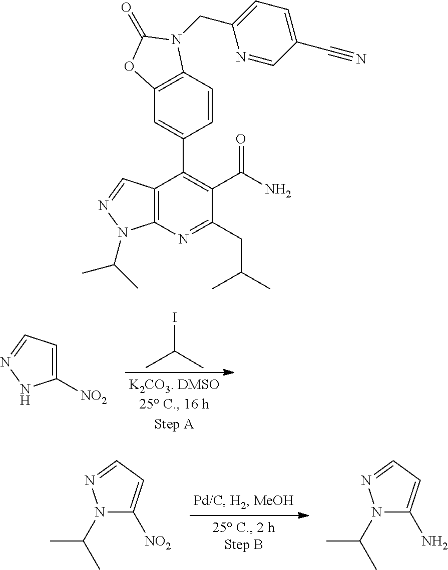

### Step A: 1-isopropyl-5-nitro-1H-pyrazole

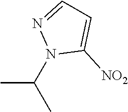

To a solution of 5-nitro-1H-pyrazole (5 g, 44.216 mmol) in 2-iodopropane (4.864 mL, 48.638 mmol) were added K₂CO₃ (7.33 g, 53.060 mmol) and DMSO (100 mL), the reaction mixture was stirred at 25° C. for 16 hrs under N₂. The mixture was added to EA (100 mL) and ice-water (200 mL), the mixture was separated and the aqueous phase was extracted with EA (50 mL×2), the combined organic layers were washed with brine (50 mL×3), dried over sodium sulfate, filtered and concentrated under vacuum. The resulting residue was purified by silica gel chromatography (eluting PE/EA=20/1) to afford 1-isopropyl-5-nitro-1H-pyrazole (1.3 g, 18.95%) as a colorless oil. LC-MS: m/z 156.3 (M+H)⁺. ¹H NMR (400 MHz, CDCl₃) δ 7.52 (d, J=2.4 Hz, 1H), 7.03 (d, J=2.4 Hz, 1H), 5.42-5.48 (m, 1H), 1.54 (d, J=6.8 Hz, 6H).

### Step B: 1-isopropyl-1H-pyrazol-5-amine

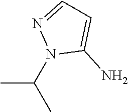

To a solution of 1-isopropyl-5-nitro-1H-pyrazole (200 mg, 1.289 mmol) in MeOH (10 mL) was added Pd/C (27.43 mg, 0.258 mmol), the reaction mixture was stirred at 25° C. for 2 hrs under H₂. The mixture was filtered and concentrated under vacuum to afford 1-isopropyl-1H-pyrazol-5-amine (150 mg, 92.97%) as a yellow oil, which was used in the next step without further purification. LC-MS: m/z 126.1 (M+H)+4-{3-[(5-cyano-2-pyridyl)methyl]-2-oxo-1,3-benzoxazol-6-yl}-6-isobutyl-1-isopropyl-1H-1,2,7-triazaindene-5-carboxamide (Compound 178)

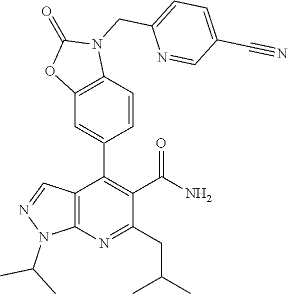 [SMILES: CC(C)Cc1nc2c(cnn2C(C)C)c(-c2ccc3c(c2)oc(=O)n3Cc2ccc(C#N)cn2)c1C(N)=O]

Compound 178 was then synthesized following the similar route of Example 26, using 1-isopropyl-1H-pyrazol-5-amine in step D. ¹H NMR (400 MHz, DMSO-d₆) δ 8.98 (d, J=1.6 Hz, 1H), 8.38 (dd, J=8.4, 2.4 Hz, 1H), 7.90 (s, 1H), 7.77 (d, J=8.4 Hz, 1H), 7.71 (m, 1H), 7.61 (d, J=1.2 Hz, 1H), 7.48 (m, 1H), 7.46 (dd, J=8.0, 1.2 Hz, 1H), 7.34 (d, J=8.4 Hz, 1H), 5.35 (s, 2H), 5.17-5.23 (m, 1H), 2.81 (d, J=6.8 Hz, 2H), 2.33-2.39 (m, 1H), 1.52 (d, J=6.8 Hz, 6H), 0.96 (d, J=6.8 Hz, 6H). LC-MS: m/z 510.3 (M+H)⁺.

### 3-[(p-fluorophenyl)methyl]-7-{3-[(5-fluoro-2-pyridyl)methyl]-2-oxo-1,3-benzoxazol-6-yl}-5-isobutyl-3-methyl-1,1-dioxo-1λ⁶-thia-4-aza-6-indancarboxamide (Compound 172)

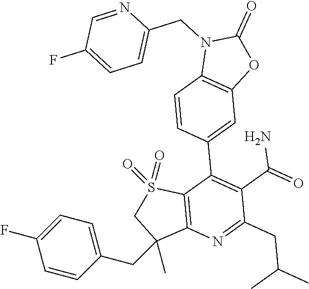 [SMILES: CC(C)Cc1nc2c(c(-c3ccc4c(c3)oc(=O)n4Cc3ccc(F)cn3)c1C(N)=O)S(=O)(=O)CC2(C)Cc1ccc(F)cc1]

Compound 172 was synthesized following the similar route of Example 26, using 4-(4-fluorobenzyl)-4-methyldihydrothiophen-3(2H)-one 1,1-dioxide in step A and 2-(bromomethyl)-5-fluoropyridine and 3-(4-fluorobenzyl)-5-isobutyl-3-methyl-7-(2-oxo-2,3-dihydrobenzo[d]oxazol-6-yl)-2,3-dihydrothieno[3,2-b]pyridine-6-carboxamide 1,1-dioxide in step D.

¹H NMR (400 MHz, DMSO-d₆) δ 8.53 (d, J=2.8 Hz, 1H), 7.86 (m, 1H), 7.76-7.81 (m, 1H), 7.60-7.65 (m, 2H), 7.41 (s, 1H), 7.25-7.30 (m, 2H), 7.17-7.21 (m, 2H), 7.11 (t, J=8.8 Hz, 2H), 5.20 (s, 2H), 3.66 (d, J=13.6 Hz, 1H), 3.42 (d, J=13.6 Hz, 1H), 3.24 (d, J=13.6 Hz, 1H), 3.16 (d, J=13.2 Hz, 1H), 2.77-2.79 (m, 2H), 2.29-2.36 (m, 1H), 1.46 (s, 3H), 0.96 (dd, J=13.2, 6.8 Hz, 6H). ¹⁹F NMR (377 MHz, DMSO-d₆) δ −116.08, −128.65. LC-MS: m/z 633.8 (M+H)⁺.

### Example 47

(S)-3-[(p-fluorophenyl)methyl]-7-{3-[(5-fluoro-2-pyridyl)methyl]-2-oxo-1,3-benzoxazol-6-yl}-5-isobutyl-3-methyl-1,1-dioxo-1λ⁶-thia-4-aza-6-indancarboxamide and (R)-3-[(p-fluorophenyl)methyl]-7-{3-[(5-fluoro-2-pyridyl)methyl]-2-oxo-1,3-benzoxazol-6-yl}-5-isobutyl-3-methyl-1,1-dioxo-1λ⁶-thia-4-aza-6-indancarboxamide (Compound 168 and Compound 167)

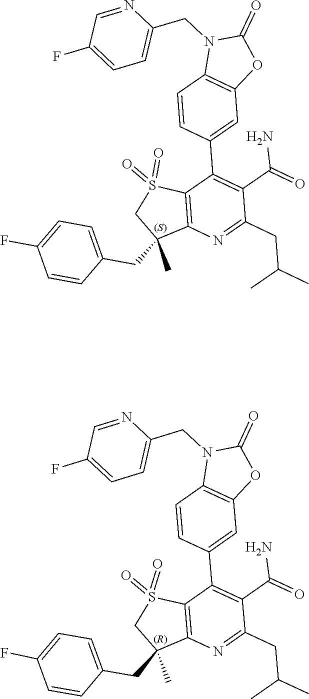

3-[(p-fluorophenyl)methyl]-7-{3-[(5-fluoro-2-pyridyl)methyl]-2-oxo-1,3-benzoxazol-6-yl}-5-isobutyl-3-methyl-1,1-dioxo-1λ⁶-thia-4-aza-6-indancarboxamide (20.0 mg, 0.030 mmol) was purified by SFC (system: Waters SFC 150; Column name: DAICEL CHIRALPAK® IC; Column size: 250*25 mm 10 um; Mobile Phase A: Supercritical CO₂; Mobile Phase B: EtOH (+0.1% 7.0 mol/l Ammonia in MEOH); Gradient: A/B=60/40; Flow rate: 120 mL/min; Column Temp: RT) to afford (S)-3-[(p-fluorophenyl)methyl]-7-{3-[(5-fluoro-2-pyridyl)methyl]-2-oxo-1,3-benzoxazol-6-yl}-5-isobutyl-3-methyl-1,1-dioxo-1λ⁶-thia-4-aza-6-indancarboxamide and (R)-3-[(p-fluorophenyl)methyl]-7-{3-[(5-fluoro-2-pyridyl)methyl]-2-oxo-1,3-benzoxazol-6-yl}-5-isobutyl-3-methyl-1,1-dioxo-1λ⁶-thia-4-aza-6-indancarboxamide.

Compound 168, enantiomer 1 (7.37 mg, 36.85%): ¹H NMR (400 MHz, DMSO-d₆) δ 8.53 (d, J=2.8 Hz, 1H), 7.85-7.86 (m, 1H), 7.76-7.81 (m, 1H), 7.63 (dd, J=8.8, 4.4 Hz, 1H), 7.59-7.60 (m, 1H), 7.41 (s, 1H), 7.25-7.30 (m, 2H), 7.17-7.21 (m, 2H), 7.11 (t, J=8.8 Hz, 2H), 5.20 (s, 2H), 3.66 (d, J=13.6 Hz, 1H), 3.41 (d, J=13.6 Hz, 1H), 3.24 (d, J=13.6 Hz, 1H), 3.16 (d, J=13.2 Hz, 1H), 2.77-2.79 (m, 2H), 2.28-2.36 (m, 1H), 1.46 (s, 3H), 0.96 (dd, J=13.2, 6.8 Hz, 6H). ¹⁹F NMR (377 MHz, DMSO-d₆) δ −116.09, −128.86. LC-MS: m/z 633.8 (M+H)⁺.

Compound 167, enantiomer 2 (9.77 mg, 48.85%): ¹H NMR (400 MHz, DMSO-d₆) δ 8.53 (d, J=2.8 Hz, 1H), 7.85-7.86 (m, 1H), 7.76-7.81 (m, 1H), 7.63 (dd, J=8.8, 4.4 Hz, 1H), 7.59 (m, 1H), 7.41 (s, 1H), 7.25-7.30 (m, 2H), 7.17-7.21 (m, 2H), 7.11 (t, J=8.8 Hz, 2H), 5.20 (s, 2H), 3.66 (d, J=13.6 Hz, 1H), 3.41 (d, J=13.6 Hz, 1H), 3.24 (d, J=13.2 Hz, 1H), 3.15 (d, J=13.2 Hz, 1H), 2.77-2.79 (m, 2H), 2.29-2.35 (m, 1H), 1.46 (s, 3H), 0.96 (dd, J=13.2, 6.8 Hz, 6H). ¹⁹F NMR (377 MHz, DMSO-d₆) δ −116.08, −128.86, LC-MS: m/z 633.8 (M+H)⁺.

### Example 49

### 7-{3-[(4-chloro-5-fluoro-2-pyridyl)methyl]-2-oxo-1,3-benzoxazol-6-yl}-3-(cyclopropylmethyl)-5-isobutyl-3-methyl-1,1-dioxo-1λ⁶-thia-4-aza-6-indancarboxamide (Compound 165)

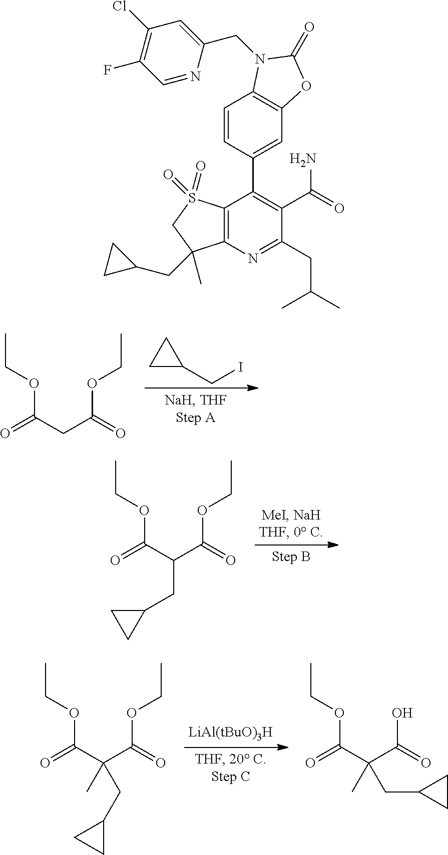

### Step A: diethyl 2-(cyclopropylmethyl) malonate

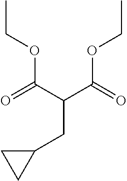

To a solution of diethyl malonate (2 g, 12.49 mmol, 1.90 mL) in THF (20 mL) was added NaH (749.14 mg, 18.73 mmol, 46.83 μL, 60% purity) at 0° C. The mixture was stirred at 0° C. for 30 min. Then (iodomethyl)cyclopropane (2.73 g, 14.98 mmol) was added dropwise. The mixture was stirred at 10° C. for 1 hr. The reaction mixture was quenched with H₂O (30 mL) at 0° C., and extracted with EtOAc (30 mL×2). The combined organic layers were concentrated under reduced pressure to afford a residue. The residue was purified by flash silica gel chromatography (0~20% EA/PE) to afford diethyl 2-(cyclopropylmethyl) malonate (0.9 g, 33.64%) as a colorless oil. LC-MS: m/z 539.5 (M+H)⁺.

### Step B: diethyl 2-(cyclopropylmethyl)-2-methylmalonate

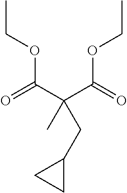 [SMILES: CCOC(=O)C(C)(CC1CC1)C(=O)OCC]

To a solution of diethyl 2-(cyclopropylmethyl) malonate (0.9 g, 4.20 mmol) in THF (10 mL) was added NaH (336.01 mg, 8.40 mmol, 60% purity) at 0° C. and stirred for 0.5 hr. Then CH₃I (894.33 mg, 6.30 mmol, 392.25 μL) was added. The mixture was stirred at 0-10° C. for 2 hrs. The reaction mixture was quenched with H₂O (20 mL) at 0° C., and then extracted with EtOAc (20 mL*2). The combined organic layers were concentrated under reduced pressure to afford a residue. The residue was purified by flash silica gel chromatography (0~20% EA/PE) to afford diethyl 2-(cyclopropylmethyl)-2-methylmalonate (700 mg, 73%) as a colorless oil. LC-MS: m/z 229.2 (M+H)⁺.

### Step C: ethyl 3-cyclopropyl-2-(hydroxymethyl)-2-methylpropanoate

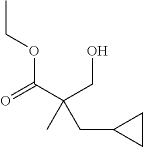

To a solution of diethyl 2-(cyclopropylmethyl)-2-methylmalonate (600 mg, 2.63 mmol) in THF (10 mL) was added lithium tritert-butoxyalumanuide (1 M, 5.78 mL). The mixture was stirred at 65° C. for 16 hrs. The reaction mixture was quenched with H₂O (0.96 mL) at 0° C., and then diluted with 15% NaOH aqueous (1 mL) and dried, filtered and concentrated under reduced pressure to afford a residue. The residue was purified by flash silica gel chromatography (0~50% EA/PE) to afford ethyl 3-cyclopropyl-2-(hydroxymethyl)-2-methylpropanoate (200 mg, 40.86%) as a colorless oil. LC-MS: m/z 187.0 (M+H)⁺. ¹H NMR (400 MHz, CDCl₃) δ 4.17 (q, J=7.2 Hz, 2H), 3.83 (d, J=11.2 Hz, 1H), 3.54 (d, J=11.2 Hz, 1H), 2.31 (br s, 1H), 1.51 (d, J=6.8 Hz, 2H), 1.28 (t, J=7.2 Hz, 3H), 1.24 (s, 3H), 0.74-0.62 (m, 1H), 0.50-0.39 (m, 2H), 0.11-0.03 (m, 2H).

### 7-{3-[(4-chloro-5-fluoro-2-pyridyl)methyl]-2-oxo-1,3-benzoxazol-6-yl}-3-(cyclopropylmethyl)-5-isobutyl-3-methyl-1,1-dioxo-1λ⁶-thia-4-aza-6-indancarboxamide (Compound 165)

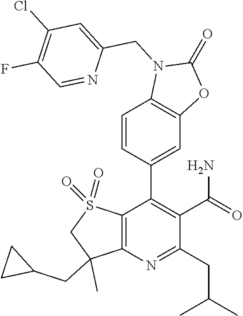 [SMILES: CC(C)Cc1nc2c(c(-c3ccc4c(c3)oc(=O)n4Cc3cc(Cl)c(F)cn3)c1C(N)=O)S(=O)(=O)CC2(C)CC1CC1]

Compound 165 was then synthesized following the similar route of Example 31, using ethyl 3-cyclopropyl-2-methyl-2-((methylthio)methyl) propanoate in step D and then following the similar route of Example 2, using 4-amino-3-(cyclopropylmethyl)-3-methyl-2,3-dihydrothiophene 1,1-dioxide and 3-((4-chloro-5-fluoropyridin-2-yl)methyl)-2-oxo-2,3-dihydrobenzo[d]oxazole-6-carbaldehyde in step B.

¹H NMR (400 MHz, CD₃OD) δ 8.49 (d, J=1.2 Hz, 1H), 7.74 (d, J=5.6 Hz, 1H), 7.48 (d, J=1.2 Hz, 1H), 7.36 (dd, J=1.6, 8.0 Hz, 1H), 7.19 (d. J=8.0 Hz, 1H), 5.19 (s, 2H), 3.89 (d, J=13.6 Hz, 1H), 3.46 (d, J=13.6 Hz, 1H), 2.86 (d, J=1.2 Hz, 2H), 2.43-2.33 (m, 1H), 2.00-1.94 (m, 1H), 1.86-1.79 (m, J=6.6, 14.0 Hz, 1H), 1.60 (s, 3H), 1.00 (t, J=6.6 Hz, 6H), 0.73-0.63 (m, 1H), 0.57-0.39 (m, 2H), 0.16-0.01 (m, 2H). ¹⁹F NMR (377 MHz, CD₃OD) δ −134.01. LC-MS: m/z 613.1 (M+H)⁺.

### Example 50

### 7-{4-[(5-chloro-2-pyridyl)methyl]-2,2-dimethyl-3-oxo-3,4-dihydro-2H-1,4-benzoxazin-7-yl}-5-(cyclopropylmethyl)-3-[2,2,2-trifluoro-1-(methoxymethyl)ethyl]-1,3,4-triaza-3H-indene-6-carboxamide (Compound 179)

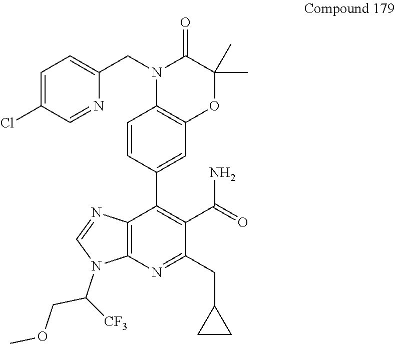

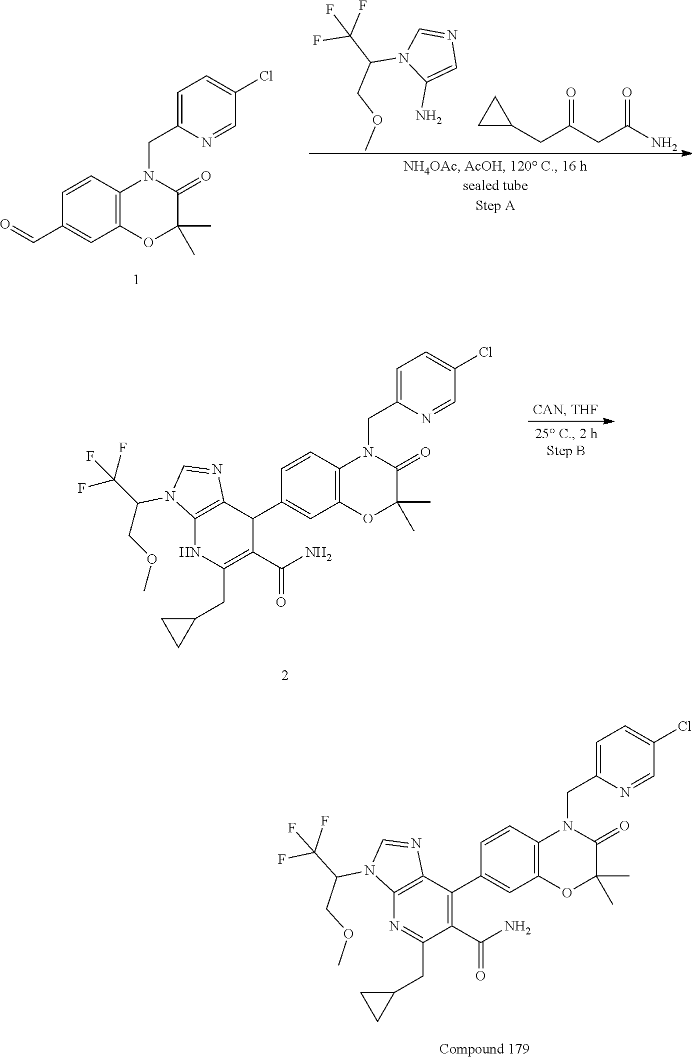 [SMILES: CC1(C)Oc2cc(C=O)ccc2N(Cc2ccc(Cl)cn2)C1=O ; COCC(n1cncc1N)C(F)(F)F ; NC(=O)CC(=O)CC1CC1 ; COCC(n1cnc2c1NC(CC1CC1)=C(C(N)=O)C2c1ccc2c(c1)OC(C)(C)C(=O)N2Cc1ccc(Cl)cn1)C(F)(F)F ; COCC(n1cnc2c(-c3ccc4c(c3)OC(C)(C)C(=O)N4Cc3ccc(Cl)cn3)c(C(N)=O)c(CC3CC3)nc21)C(F)(F)F]

### Step A: 7-(4-((5-chloropyridin-2-yl)methyl)-2,2-dimethyl-3-oxo-3,4-dihydro-2H-benzo[b][1,4]oxazin-7-yl)-5-(cyclopropylmethyl)-3-(1,1,1-trifluoro-3-methoxypropan-2-yl)-4,7-dihydro-3H-imidazo[4,5-b]pyridine-6-carboxamide

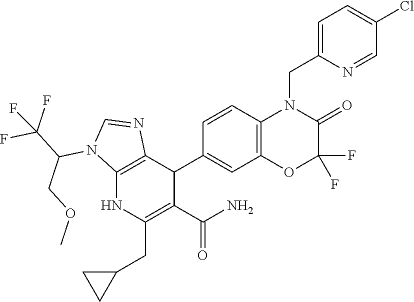 [SMILES: COCC(n1cnc2c1NC(CC1CC1)=C(C(N)=O)C2c1ccc2c(c1)OC(F)(F)C(=O)N2Cc1ccc(Cl)cn1)C(F)(F)F]

A mixture of 1-(1,1,1-trifluoro-3-methoxypropan-2-yl)-1H-imidazol-5-amine (60 mg, 0.287 mmol), 4-[(5-chloropyridin-2-yl)methyl]-2,2-dimethyl-3-oxo-3,4-dihydro-2H-benzo[b][1,4]oxazine-7-carbaldehyde (94.88 mg, 0.287 mmol), 4-cyclopropyl-3-oxobutanamide (121.48 mg, 0.861 mmol) and NH₄OAC (44.22 mg, 0.574 mmol) in AcOH (3 mL) was stirred at 120° C. for 16 h under N₂ atmosphere. The reaction mixture was concentrated to afford 7-(4-((5-chloropyridin-2-yl)methyl)-2,2-dimethyl-3-oxo-3,4-dihydro-2H-benzo[b][1,4]oxazin-7-yl)-5-(cyclopropylmethyl)-3-(1,1,1-trifluoro-3-methoxypropan-2-yl)-4,7-dihydro-3H-imidazo[4,5-b]pyridine-6-carboxamide (62 mg, 27.02%) as a yellow oil, which was used in the next step without further purification. LC-MS: m/z 636.4 (M+H)⁺.

### Step B: 7-{4-[(5-chloro-2-pyridyl)methyl]-2,2-dimethyl-3-oxo-3,4-dihydro-2H-1,4-benzoxazin-7-yl}-5-(cyclopropylmethyl)-3-[2,2,2-trifluoro-1-(methoxymethyl)ethyl]-1,3,4-triaza-3H-indene-6-carboxamide (Compound 179)

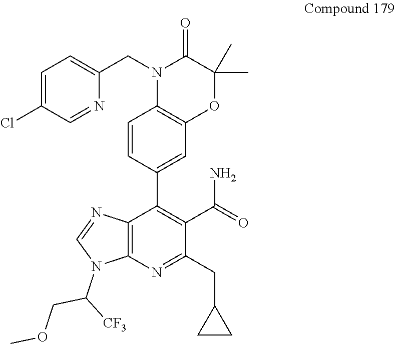

To a solution of 7-(4-((5-chloropyridin-2-yl)methyl)-2,2-dimethyl-3-oxo-3,4-dihydro-2H-benzo[b][1,4]oxazin-7-yl)-5-(cyclopropylmethyl)-3-(1,1,1-trifluoro-3-methoxypropan-2-yl)-4,7-dihydro-3H-imidazo[4,5-b]pyridine-6-carboxamide (62 mg, 0.095 mmol) in THF (5 mL) was added CAN (127.48 mg, 0.233 mmol) under N₂. The reaction mixture was stirred at 25° C. for 2 hours under N₂. The mixture was added aq. Na₂CO₃ to pH=8. The resulting residue was poured into H₂O (50 mL) and extracted with EA (50 mL×3). The combined organics was washed brine (50 mL) and dried over sodium sulfate, filtered and concentrated under vacuum. The resulting residue was purified by prep-TLC (eluting DCM/MeOH=10/1) and prep-HPLC (Waters 3767/Qda Column: SunFire Sunfire C18, 19*250 mm*10 um; Mobile Phase A: 0.1% FA/H₂O, B: ACN; Flow rate: 20 ml/min; Gradient: 53%-58%; Retention Time: 8.2-8.8 min of 16 min) to afford 7-{4-[(5-chloro-2-pyridyl)methyl]-2,2-dimethyl-3-oxo-3,4-dihydro-2H-1,4-benzoxazin-7-yl}-5-(cyclopropylmethyl)-3-[2,2,2-trifluoro-1-(methoxymethyl)ethyl]-1,3,4-triaza-3H-indene-6-carboxamide (6.51 mg, 9.28%).

¹H NMR (400 MHz, DMSO-d₆) δ 8.58 (d, J=2.0 Hz, 1H), 8.40 (s, 1H), 7.94 (dd, J₁=2.4 Hz, J₂=8.4 Hz, 1H), 7.77 (d, J=1.6 Hz, 1H), 7.49 (d, J=1.6 Hz, 1H), 7.38-7.40 (m, 2H), 7.34 (dd, J₁=2.0 Hz, J₂=8.4 Hz, 1H), 7.08 (d, J=8.4 Hz, 1H), 5.25 (s, 2H), 4.54-4.66 (m, 3H), 3.39 (s, 3H), 2.77 (d, J=6.8 Hz, 2H), 1.51 (s, 6H), 1.23-1.29 (m, 1H), 0.42-0.44 (m, 2H), 0.28-0.29 (m, 2H). ¹⁹F NMR (376 MHz, DMSO-d₆) δ −74.80. LC-MS: m/z 643.3 (M+H)⁺.

### 7-{4-[(5-chloro-2-pyridyl)methyl]-2,2-difluoro-3-oxo-3,4-dihydro-2H-1,4-benzoxazin-7-yl}-5-(cyclopropylmethyl)-3-[2,2,2-trifluoro-1-(methoxymethyl)ethyl]-1,3,4-triaza-3H-indene-6-carboxamide (Compound 180)

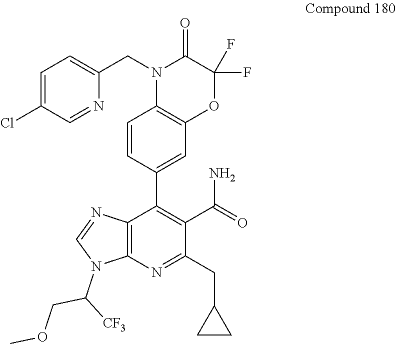 [SMILES: COCC(n1cnc2c(-c3ccc4c(c3)OC(F)(F)C(=O)N4Cc3ccc(Cl)cn3)c(C(N)=O)c(CC3CC3)nc21)C(F)(F)F]

Compound 180 was synthesized following the similar route of Example 50, using 4-((5-chloropyridin-2-yl)methyl)-2,2-difluoro-3-oxo-3,4-dihydro-2H-benzo[b][1,4]oxazine-7-carbaldehyde in step A.

¹H NMR (400 MHz, DMSO-d₆) δ 8.58 (d, J=2.4 Hz, 1H), 8.43 (s, 1H), 7.99 (dd, J₁=2.8 Hz, J₂=8.4 Hz, 1H), 7.85 (s, 1H), 7.65 (d, J=1.6 Hz, 1H), 7.59 (d, J=8.4 Hz, 1H), 7.52-7.55 (m, 2H), 7.40 (d, J=8.4 Hz, 1H), 5.43 (s, 2H), 4.56-4.68 (m, 3H), 3.40 (s, 3H), 2.79 (d, J=6.8 Hz, 2H), 1.23-1.28 (m, 1H), 0.43-0.45 (m, 2H), 0.29-0.30 (m, 2H). ¹⁹F NMR (376 MHz, DMSO-d₆) δ −74.61, −74.77. LC-MS: m/z 651.3 (M+H)⁺.

### Example 51

### 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[1-(trifluoromethyl)cyclopropyl]-1,2,7-triaza-1H-indene-5-carboxamide (Compound 181)

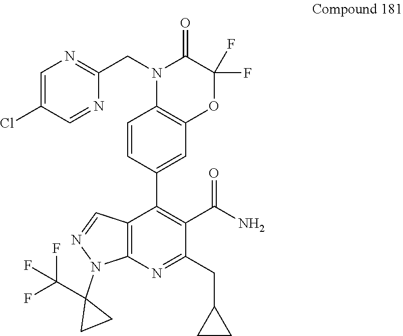 [SMILES: NC(=O)c1c(CC2CC2)nc2c(cnn2C2(C(F)(F)F)CC2)c1-c1ccc2c(c1)OC(F)(F)C(=O)N2Cc1ncc(Cl)cn1]

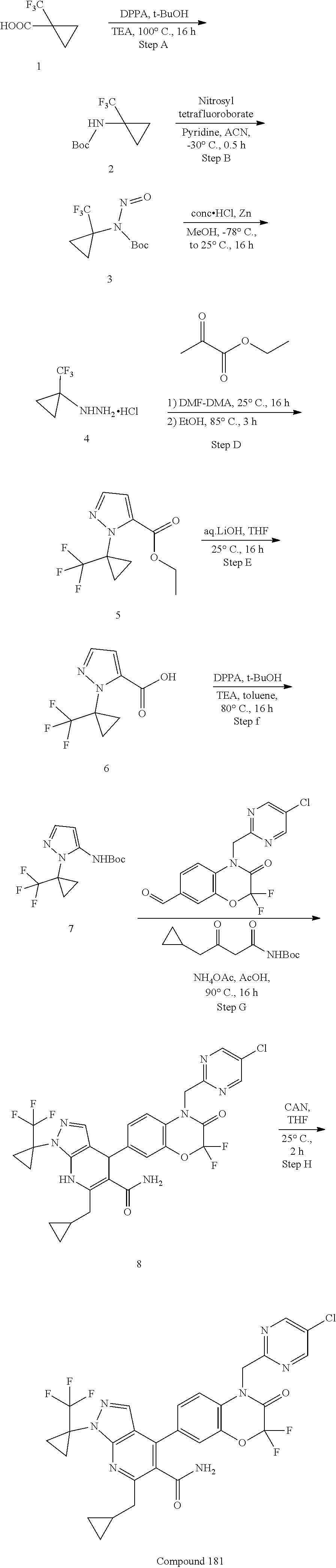

### Step A: tert-butyl(1-(trifluoromethyl)cyclopropyl)carbamate

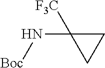 [SMILES: CC(C)(C)OC(=O)NC1(C(F)(F)F)CC1]

To a solution of 1-(trifluoromethyl)cyclopropane-1-carboxylic acid (5.0 g, 32.449 mmol) in tort-Butanol (60 mL) was added TEA (4.961 mL, 35.693 mmol) and diphenyl azidophosphonate (13394.77 mg, 48.673 mmol). The reaction mixture was stirred at 100° C. for 16 h under Ar. The reaction mixture was poured into EA (250 mL), washed with 5% citric acid (200 mL), aq. NaHCO₃ (100 mL) and brine (100 mL), dried over sodium sulfate, filtered and concentrated under vacuum. The resulting residue was stirred with PE/EA (10/1, 100 mL), a white solid was formed and filtered. The filtrate was concentrated under vacuum, the resulting residue was stirred with PE (50 mL) at 0° C. for 10 min, the mixture was 10 filtered, the cake was dried under vacuum to afford tert-butyl(1-(trifluoromethyl)cyclopropyl)carbamate (4.17 g, 57.06%) as a white solid. ¹H NMR (400 MHz, DMSO-d₆) δ 7.79 (s, 1H), 1.37 (s, 9H), 1.14-1.17 (m, 2H), 0.99 (m, 2H). LC-MS: m/z 169.9 (M+H−56)⁺.

### Step B: tert-butyl nitroso(1-(trifluoromethyl)cyclopropyl)carbamate

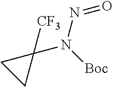 [SMILES: CC(C)(C)OC(=O)N(N=O)C1(C(F)(F)F)CC1]

To a solution of tert-butyl(1-(trifluoromethyl)cyclopropyl)carbamate (3.80 g, 16.873 mmol) in ACN (60 mL) and Pyridine (2.729 mL, 33.746 mmol) were added Nitrosonium tetrafluoroborate (230 mg, 1.936 mmol) at −30° C. under N₂, the mixture was stirred at −30° C. for 30 min. The mixture was added to EA (100 mL), washed with aq. citric acid (5%, 50 mL), aq. NaHCO₃ (100 mL) and brine (50 mL), dried over sodium sulfate, filtered and concentrated under vacuum. The resulting residue was purified by silica gel chromatography (eluting PE/EA=5/1) to afford tert-butyl nitroso (1-(trifluoromethyl)cyclopropyl)carbamate (3.45 g, 80.43%) as a yellow oil. ¹H NMR (400 MHz, CDCl₃) δ 1.58 (s, 9H), 1.49 (m, 2H), 0.80 (m, 2H).

### Step C: (1-(trifluoromethyl)cyclopropyl)hydrazine hydrochloride

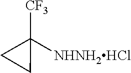

To a solution of tert-butyl nitroso (1-(trifluoromethyl)cyclopropyl)carbamate (3.5 g, 13.768 mmol) in MeOH (120 mL) was stirred at −78° C. for 10 min under N₂. conc. HCl (11.473 mL, 137.681 mmol) was added, followed by Zn (9001.61 mg, 137.681 mmol). The mixture was allowed to warm to 25° C. and stirred at 25° C. for 16 h. The mixture was filtered and the filtrate was concentrated under vacuum to afford (1-(trifluoromethyl)cyclopropyl)hydrazine hydrochloride (1.93 g, 100%) as a yellow oil, which was used in the next step without further purification. LC-MS: m/z 141.0 (M+H-HCl)⁺.

### Step D: ethyl 1-(1-(trifluoromethyl)cyclopropyl)-1H-pyrazole-5-carboxylate

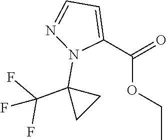 [SMILES: CCOC(=O)c1ccnn1C1(C(F)(F)F)CC1]

A mixture of ethyl 2-oxopropanoate (20 g, 172.236 mmol) in DMF-DMA (20.50 g, 172.236 mmol) was stirred at 25° C. for 16 h under N₂. The mixture was concentrated under vacuum, the resulting residue was purified by silica gel chromatography (eluting DCM/MeOH=50/1) to afford ethyl(3E)-4-(dimethylamino)-2-oxobut-3-enoate (9.4 g, 31.88%) as a brown oil. Then to a solution of ethyl(3E)-4-(dimethylamino)-2-oxobut-3-enoate (9.4 g, 55.1 mmol) in EtOH (1000 mL) was added (1-(trifluoromethyl)cyclopropyl)hydrazine hydrochloride (3.86 g, 27.55 mmol), the reaction mixture was stirred at 85° C. for 3 h under N₂. The reaction mixture was concentrated under vacuum. The resulting residue was purified by silica gel chromatography (eluting PE/EA=1/1) to afford ethyl 1-(1-(trifluoromethyl)cyclopropyl)-1H-pyrazole-5-carboxylate (6.1 g, 89.20%) as a yellow oil. LC-MS: m/z 249.2 (M+H)⁺.

### Step E: 1-(1-(trifluoromethyl)cyclopropyl)-1H-pyrazole-5-carboxylic acid

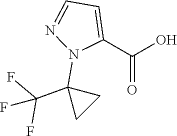 [SMILES: O=C(O)c1ccnn1C1(C(F)(F)F)CC1]

To a solution of ethyl 1-(1-(trifluoromethyl)cyclopropyl)-1H-pyrazole-5-carboxylate (6.1 g, 24.576 mmol) in THF (100 mL) were added LiOH (10.32 g, 245.76 mmol) and H₂O (100 mL), the reaction mixture was stirred at 25° C. for 16 h. The mixture was adjusted to pH=3 by 1N HCl, extracted with EA (200 mL×2), the combined organic layers were dried over sodium sulfate, filtered and concentrated in vacuum to afford 1-(1-(trifluoromethyl)cyclopropyl)-1H-pyrazole-5-carboxylic acid (1.05 g, 18.21%) as a yellow oil, which was used in the next step without further purification. LC-MS: m/z 221.0 (M+H)⁺.

### Step F: tert-butyl(1-(1-(trifluoromethyl)cyclopropyl)-1H-pyrazol-5-yl)carbamate

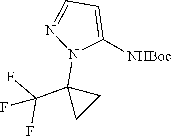 [SMILES: CC(C)(C)OC(=O)Nc1ccnn1C1(C(F)(F)F)CC1]

To a solution of 1-(1-(trifluoromethyl)cyclopropyl)-1H-pyrazole-5-carboxylic acid (1.05 g, 4.769 mmol) in Toluene (20 mL) were added TEA (1.127 mL, 8.108 mmol), t-BuOH (3.491 mL, 38.156 mmol) and DPPA (1.97 g, 7.154 mmol). The reaction mixture was stirred at 80° C. for 16 h under N₂. The reaction mixture was concentrated in vacuum. The resulting residue was purified by silica gel chromatography (eluting PE/EA=7/1) to afford tert-butyl(1-(1-(trifluoromethyl)cyclopropyl)-1H-pyrazol-5-yl)carbamate (890 mg, 64.07%) as a yellow solid. ¹H NMR (400 MHz, DMSO-d₆) δ 8.94 (s, 1H), 7.46 (d, J=2 Hz, 1H), 6.19 (d, J=1.6 Hz, 1H), 1.51-1.61 (m, 4H), 1.44 (s, 9H). LC-MS: m/z 292.2 (M+H)⁺.

### Step G: 4-(4-((5-chloropyrimidin-2-yl)methyl)-2,2-difluoro-3-oxo-3,4-dihydro-2H-benzo[b][1,4]oxazin-7-yl)-6-(cyclopropylmethyl)-1-(1-(trifluoromethyl)cyclopropyl)-4,7-dihydro-1H-pyrazolo[3,4-b]pyridine-5-carboxamide

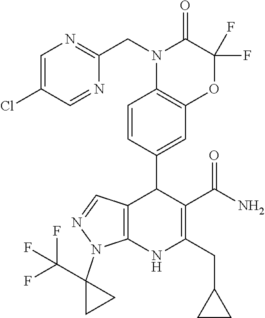 [SMILES: NC(=O)C1=C(CC2CC2)Nc2c(cnn2C2(C(F)(F)F)CC2)C1c1ccc2c(c1)OC(F)(F)C(=O)N2Cc1ncc(Cl)cn1]

A mixture of 4-((5-chloropyrimidin-2-yl)methyl)-2,2-difluoro-3-oxo-3,4-dihydro-2H-benzo[b][1,4]oxazine-7-carbaldehyde (6.0 g, 17.664 mmol), tert-butyl(1-(1-(trifluoromethyl)cyclopropyl)-1H-pyrazol-5-yl)carbamate (5.14 g, 17.664 mmol), tert-butyl(4-cyclopropyl-3-oxobutanoyl)carbamate (4.26 g, 17.664 mmol) and NH₄OAc (2.72 g, 35.327 mmol) in AcOH (220 mL) was stirred at 90° C. for 16 h under N₂. The reaction mixture was concentrated under vacuum to afford 4-(4-((5-chloropyrimidin-2-yl)methyl)-2,2-difluoro-3-oxo-3,4-dihydro-2H-benzo[b][1,4]oxazin-7-yl)-6-(cyclopropylmethyl)-1-(1-(trifluoromethyl)cyclopropyl)-4,7-dihydro-1H-pyrazolo[3,4-b]pyridine-5-carboxamide (11.23 g, 100%) as a yellow oil, which was used in the next step without further purification. LC-MS: m/z 636.4 (M+H)⁺.

### Step H: 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[1-(trifluoromethyl)cyclopropyl]-1,2,7-triaza-1H-indene-5-carboxamide (Compound 181)

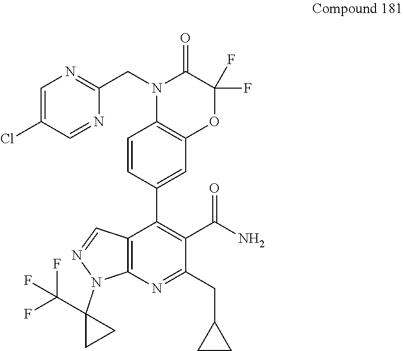 [SMILES: NC(=O)c1c(CC2CC2)nc2c(cnn2C2(C(F)(F)F)CC2)c1-c1ccc2c(c1)OC(F)(F)C(=O)N2Cc1ncc(Cl)cn1]

A mixture of 4-(4-((5-chloropyrimidin-2-yl)methyl)-2,2-difluoro-3-oxo-3,4-dihydro-2H-benzo[b][1,4]oxazin-7-yl)-6-(cyclopropylmethyl)-1-(1-(trifluoromethyl)cyclopropyl)-4,7-dihydro-1H-pyrazolo[3,4-b]pyridine-5-carboxamide (22.47 g, 35.328 mmol) and CAN (38.74 g, 70.656 mmol) in THF (230 mL) was stirred at 25° C. for 2 h. The reaction mixture was added to EA (200 mL) and aq. NaHCO₃ (200 mL), the mixture was stirred at rt for 30 min. The organic layer was separated and washed with brine (100 mL), dried over sodium sulfate, filtered and concentrated under vacuum. The resulting residue was purified by silica gel chromatography (eluting PE/EA=2/1) to give a crude. The crude (7.2 g) was added to EtOH (50 mL), the mixture was stirred at reflux for 2 h, then allowed to cool down to rt and stirred at rt for 18 h. The mixture was filtered and the cake was dried under reduced pressure at 50° C. for 18 h to afford 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[1-(trifluoromethyl)cyclopropyl]-1,2,7-triaza-1H-indene-5-carboxamide (5.2 g, 23.25%).

¹H NMR (400 MHz, DMSO-d₆) δ 8.98 (s, 2H), 8.06 (s, 1H), 7.90 (d, J=1.6 Hz, 1H), 7.65 (d, J=1.2 Hz, 1H), 7.63 (d, J=2.0 Hz, 1H), 7.43-7.47 (m, 2H), 5.51 (s, 2H), 2.82 (d, J=6.8 Hz, 2H), 1.80-1.84 (m, 2H), 1.68 (m, 2H), 1.23-1.30 (m, 1H), 0.44-0.48 (m, 2H), 0.32-0.34 (m, 2H). ¹⁹F NMR (376 MHz, DMSO-d₆) δ −74.96, −74.83. LC-MS: m/z 634.5 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[1-(trifluoromethyl)cyclopropyl]-1,2,7-triaza-1H-indene-5-carboxamide (Compound 182)

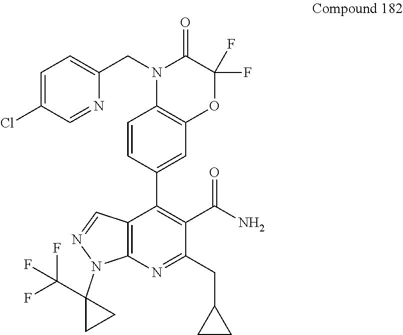 [SMILES: NC(=O)c1c(CC2CC2)nc2c(cnn2C2(C(F)(F)F)CC2)c1-c1ccc2c(c1)OC(F)(F)C(=O)N2Cc1ccc(Cl)cn1]

Compound 182 was synthesized following the similar route of Example 51, using 4-((5-chloropyridin-2-yl)methyl)-2,2-difluoro-3-oxo-3,4-dihydro-2H-benzo[b][1,4]oxazine-7-carbaldehyde in step G.

¹H NMR (400 MHz, DMSO-d₆) δ 8.59 (d, J=2.4 Hz, 1H), 8.05 (s, 1H), 8.00 (dd, J₁=2.4 Hz, J₂=8.4 Hz, 1H), 7.88 (d, J=1.6 Hz, 1H), 7.63 (d, J=1.6 Hz, 1H), 7.59-7.61 (m, 2H), 7.43-7.49 (m, 2H), 5.43 (s, 2H), 2.82 (d, J=7.2 Hz, 2H), 1.80-1.83 (m, 2H), 1.67 (m, 2H), 1.23-1.30 (m, 1H), 0.43-0.48 (m, 2H), 0.30-0.34 (m, 2H). ¹⁹F NMR (376 MHz, DMSO-d₆) δ −70.97, −74.39. LC-MS: m/z 633.4 (M+H)⁺.

### Example 52

### 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[1-(difluoromethyl)cyclopropyl]-1,2,7-triaza-1H-indene-5-carboxamide (Compound 183)

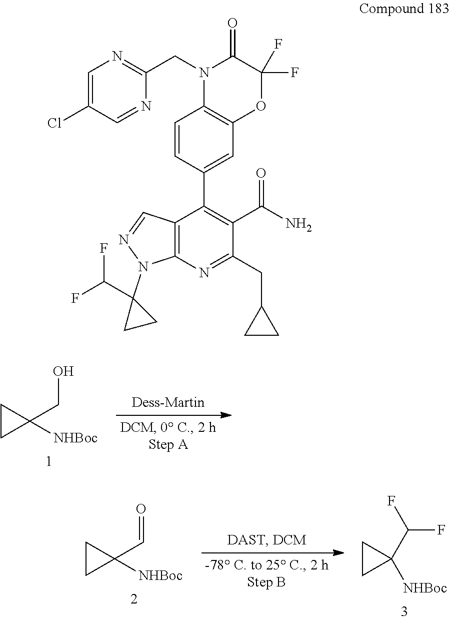 [SMILES: NC(=O)c1c(CC2CC2)nc2c(cnn2C2(C(F)F)CC2)c1-c1ccc2c(c1)OC(F)(F)C(=O)N2Cc1ncc(Cl)cn1 ; CC(C)(C)OC(=O)NC1(CO)CC1 ; CC(C)(C)OC(=O)NC1(C=O)CC1 ; CC(C)(C)OC(=O)NC1(C(F)F)CC1]

### Step A: tert-butyl(1-formylcyclopropyl)carbamate

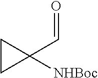 [SMILES: CC(C)(C)OC(=O)NC1(C=O)CC1]

To a solution of tert-butyl(1-(hydroxymethyl)cyclopropyl)carbamate (5 g, 26.704 mmol) in DCM (200 mL) were added DESS-MARTIN (13.59 g, 32.044 mmol) at 0° C., the reaction mixture was stirred at 0° C. for 2 h under N₂. The reaction mixture was poured into H₂O (50.0 mL) and extracted with DCM (50 mL×2). The combined organics were washed brine (20 mL) and dried over sodium sulfate, filtered and concentrated under vacuum. The resulting residue was concentrated to afford tert-butyl(1-formylcyclopropyl)carbamate (4.9 g, 99.07%) as a yellow oil. LC-MS: m/z 186.2 (M+H)⁺.

### Step B: tert-butyl(1-(difluoromethyl)cyclopropyl)carbamate

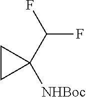 [SMILES: CC(C)(C)OC(=O)NC1(C(F)F)CC1]

To a solution of tert-butyl(1-formylcyclopropyl)carbamate (4.95 g, 26.725 mmol) in DCM (200 mL) were added DAST (7.062 mL, 53.450 mmol) at −78° C. dropwise slowly, the reaction mixture was allowed to warm to 25° C. and stirred at 25° C. for 2 h under N₂. The reaction mixture was poured into H₂O (50.0 mL) and extracted with DCM (50 mL×2). The combined organics was washed brine (20 mL) and dried over sodium sulfate, filtered and concentrated under vacuum. The resulting residue was purified by silica gel column chromatography (eluting PE/EA=20/1) to afford tert-butyl(1-(difluoromethyl)cyclopropyl)carbamate (1.1 g, 19.86%) as a yellow oil. ¹H NMR (400 MHz, DMSO-d₆) δ 7.51 (s, 1H), 5.74-6.03 (m, 1H), 1.35 (s, 9H), 0.91-0.94 (m, 2H), 0.79 (s, 2H).

### 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[1-(difluoromethyl)cyclopropyl]-1,2,7-triaza-1H-indene-5-carboxamide (Compound 183)

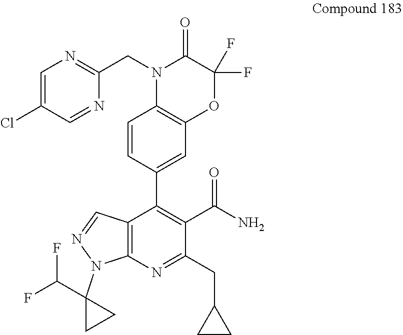 [SMILES: NC(=O)c1c(CC2CC2)nc2c(cnn2C2(C(F)F)CC2)c1-c1ccc2c(c1)OC(F)(F)C(=O)N2Cc1ncc(Cl)cn1]

Compound 183 was then synthesized following the similar route of Example 51, using tert-butyl(1-(difluoromethyl)cyclopropyl)carbamate in step B.

¹H NMR (400 MHz, DMSO-d₆) δ 8.98 (s, 2H), 7.98 (s, 1H), 7.87 (s, 1H), 7.62 (d, J=1.6 Hz, 2H), 7.43-7.48 (m, 2H), 6.17 (t, J=55 Hz, 1H), 5.51 (s, 2H), 2.81 (d, J=6.8 Hz, 2H), 1.56-1.60 (m, 2H), 1.50-1.53 (m, 2H), 1.24-1.30 (m, 1H), 0.43-0.47 (m, 2H), 0.30-0.34 (m, 2H). ¹⁹F NMR (376 MHz, DMSO-d₆) δ −74.83, −119.31. LC-MS: m/z 616.4 (M+H)⁺.

### 4-{4-[(5-chloro-2-pyridyl)methyl]-2,2-dimethyl-3-oxo-3,4-dihydro-2H-1,4-benzoxazin-7-yl}-6-(cyclopropylmethyl)-1-[1-(trifluoromethyl)cyclopropyl]-1,2,7-triaza-1H-indene-5-carboxamide (Compound 184)

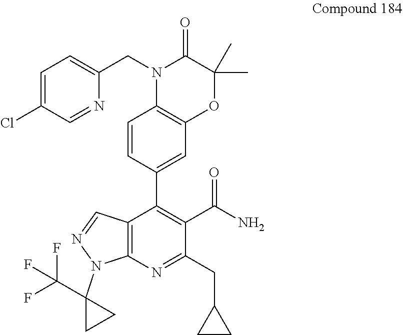 [SMILES: CC1(C)Oc2cc(-c3c(C(N)=O)c(CC4CC4)nc4c3cnn4C3(C(F)(F)F)CC3)ccc2N(Cc2ccc(Cl)cn2)C1=O]

Compound 184 was synthesized following the similar route of Example 51, using 4-[(5-chloropyridin-2-yl)methyl]-2,2-dimethyl-3-oxo-3,4-dihydro-2H-benzo[b][1,4]oxazine-7-carbaldehyde in step G.

¹H NMR (400 MHz, DMSO-d₆) δ 8.59 (d, J=2.4 Hz, 1H), 8.03 (s, 1H), 7.94 (dd, J₁=2.4 Hz, J₂=8.4 Hz, 1H), 7.80 (m, 1H), 7.57 (d, J=2 Hz, 1H), 7.40 (d, J=8.4 Hz, 1H), 7.28 (d, J=2 Hz, 1H), 7.23 (dd, J₁=1.6 Hz, J₂=8 Hz, 1H), 7.13 (d, J=8.4 Hz, 1H), 5.25 (s, 2H), 2.80 (d, J=6.8 Hz, 2H), 1.79-1.82 (m, 2H), 1.65-1.67 (m, 2H), 1.51 (s, 6H), 1.24-1.28 (m, 1H), 0.42-0.47 (m, 2H), 0.30-0.33 (m, 2H). ¹⁹F NMR (376 MHz, DMSO-d₆) δ −70.96. LC-MS: m/z 625.4 (M+H)⁺.

### 4-{4-[(5-chloro-2-pyridyl)methyl]-2,2-dimethyl-3-oxo-3,4-dihydro-2H-1,4-benzoxazin-7-yl}-6-(cyclopropylmethyl)-1-(2,2,2-trifluoroethyl)-1,2,7-triaza-1H-indene-5-carboxamide (Compound 185)

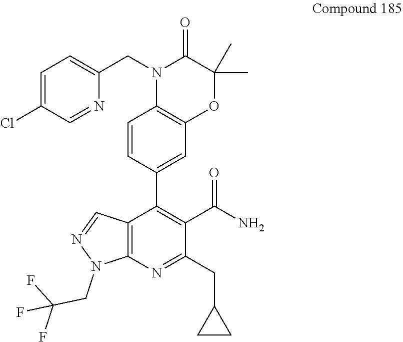 [SMILES: CC1(C)Oc2cc(-c3c(C(N)=O)c(CC4CC4)nc4c3cnn4CC(F)(F)F)ccc2N(Cc2ccc(Cl)cn2)C1=O]

Compound 185 was synthesized following the similar route of Example 46, using 2,2,2-trifluoroethyl trifluoromethanesulfonate in step A and then synthesized following the similar route of Example 51, using 4-[(5-chloropyridin-2-yl)methyl]-2,2-dimethyl-3-oxo-3,4-dihydro-2H-benzo[b][1,4]oxazine-7-carbaldehyde and 1-(2,2,2-trifluoroethyl)-1H-pyrazol-5-amine in step G.

¹H NMR (400 MHz, CD₃OD) δ 8.52 (d, J=2.4 Hz, 1H), 8.00 (s, 1H), 7.83 (dd, J=8.4, 2.4 Hz, 1H), 7.35 (d, J=8.8 Hz, 1H), 7.32 (d, J=2.0 Hz, 1H), 7.26 (dd, J=8.4, 2.0 Hz, 1H), 7.15 (d, J=8.4 Hz, 1H), 5.30 (s, 2H), 5.24 (q, J=8.8 Hz, 2H), 2.93 (d, J=6.8 Hz, 2H), 1.58 (s, 6H), 1.31-1.39 (m, 1H), 0.47-0.56 (m, 2H), 0.29-0.39 (m, 2H). ¹⁹F NMR (376 MHz, CD₃OD) δ −72.32. LC-MS: m/z 599.1 (M+H)⁺.

### 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(S)-2,2,2-trifluoro-1-methylethyl]-1,2,7-triaza-1H-indene-5-carboxamide and 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(R)-2,2,2-trifluoro-1-methylethyl]-1,2,7-triaza-1H-indene-5-carboxamide (Compound 186 and Compound 187)

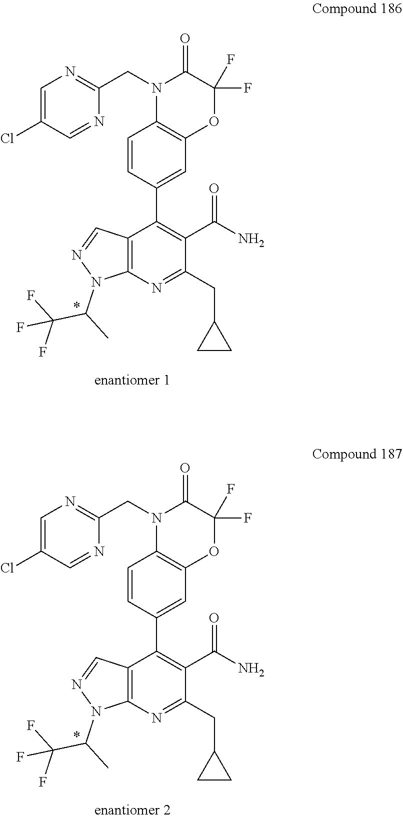 [SMILES: CC(n1ncc2c(-c3ccc4c(c3)OC(F)(F)C(=O)N4Cc3ncc(Cl)cn3)c(C(N)=O)c(CC3CC3)nc21)C(F)(F)F ; CC(n1ncc2c(-c3ccc4c(c3)OC(F)(F)C(=O)N4Cc3ncc(Cl)cn3)c(C(N)=O)c(CC3CC3)nc21)C(F)(F)F]

4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(2,2,2-trifluoro-1-methylethyl]-1,2,7-triaza-1H-indene-5-carboxamide which was synthesized following the similar route of Example 51, using 3,3,3-trifluoro-2-methylpropanoic acid in step A was purified by SFC (system: Waters SFC 150; Column name: DAICEL CHIRALCEL® OD; Column size: 250*25 mm 10 μm; Mobile Phase A: Supercritical CO₂; Mobile Phase B: MEOH (+0.1% 7.0 mol/L Ammonia in MeOH); Gradient: A/B=85:15; Flow rate: 120 mL/min; Column Temp: RT) to afford 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(S)-2,2,2-trifluoro-1-methylethyl]-1,2,7-triaza-1H-indene-5-carboxamide and 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(R)-2,2,2-trifluoro-1-methylethyl]-1,2,7-triaza-1H-indene-5-carboxamide.

Compound 186, enantiomer 1: Rt=2.84 min. ¹H NMR (400 MHz, DMSO-d₆) δ 8.98 (s, 2H), 8.10 (s, 1H), 7.90 (s, 1H), 7.65 (d, J=1.6 Hz, 2H), 7.50 (dd, J₁=1.6 Hz, J₂=8.4 Hz, 1H), 7.45 (d, J=8.4 Hz, 1H), 5.82-5.90 (m, 1H), 5.51 (s, 2H), 2.76-2.89 (m, 2H), 1.82 (d, J=7.2 Hz, 3H), 1.28-1.31 (m, 1H), 0.44-0.47 (m, 2H), 0.31-0.32 (m, 2H). ¹⁹F NMR (376 MHz, DMSO-d₆) δ −73.99, −74.84. LC-MS: m/z 622.4 (M+H)⁺.

Compound 187, enantiomer 2: Rt=3.43 min. ¹H NMR (400 MHz, DMSO-d₆) δ 8.98 (s, 2H), 8.10 (s, 1H), 7.90 (s, 1H), 7.65 (d, J=1.6 Hz, 2H), 7.50 (dd, J₁=1.6 Hz, J₂=8.4 Hz, 1H), 7.45 (d, J=8.8 Hz, 1H), 5.82-5.90 (m, 1H), 5.51 (s, 2H), 2.76-2.89 (m, 2H), 1.82 (d, J=7.2 Hz, 3H), 1.28-1.32 (m, 1H), 0.44-0.47 (m, 2H), 0.31-0.32 (m, 2H). ¹⁹F NMR (376 MHz, DMSO-d₆) δ −73.99, −74.84. LC-MS: m/z 622.3 (M+H)⁺.

### 4-{4-[(5-cyano-2-pyridyl)methyl]-2,2-dimethyl-3-oxo-3,4-dihydro-2H-1,4-benzoxazin-7-yl}-6-isobutyl-1-[1-(trifluoromethyl)cyclopropyl]-1,2,7-triaza-1H-indene-5-carboxamide (Compound 188)

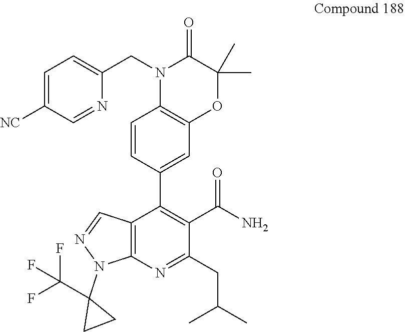 [SMILES: CC(C)Cc1nc2c(cnn2C2(C(F)(F)F)CC2)c(-c2ccc3c(c2)OC(C)(C)C(=O)N3Cc2ccc(C#N)cn2)c1C(N)=O]

Compound 188 was synthesized following the similar route of Example 2, using 4-[(5-cyanopyridin-2-yl)methyl]-2,2-dimethyl-3-oxo-3,4-dihydro-2H-benzo[b][1,4]oxazine-7-carbaldehyde and tert-butyl(1-(1-(trifluoromethyl)cyclopropyl)-1H-pyrazol-5-yl)carbamate in step B.

¹H NMR (400 MHz, DMSO-d₆) δ 8.98 (d, J=2 Hz, 1H), 8.32 (dd, J₁=2 Hz, J₂=8.4 Hz, 1H), 8.02 (s, 1H), 7.76 (d, J=1.6 Hz, 1H), 7.58 (d, J=8 Hz, 1H), 7.55 (d, J=2 Hz, 1H), 7.28 (d, J=1.6 Hz, 1H), 7.22 (dd, J₁=1.6 Hz, J₂=8 Hz, 1H), 7.11 (d, J=8.4 Hz, 1H), 5.35 (s, 2H), 2.81 (d, J=6.8 Hz, 2H), 2.31-2.38 (m, 1H), 1.78-1.81 (m, 2H), 1.64-1.66 (m, 2H), 1.52 (s, 6H), 0.95 (d, J=6.4 Hz, 6H). ¹⁹F NMR (376 MHz, DMSO-d₆) δ −71.01. LC-MS: m/z 618.4 (M+H)⁺.

### 4-{4-[(5-chloro-2-pyridyl)methyl]-2,2-dimethyl-3-oxo-3,4-dihydro-2H-1,4-benzoxazin-7-yl}-6-isobutyl-1-(2,2,2-trifluoro-1-methylethyl)-1,2,7-triaza-1H-indene-5-carboxamide (compound 189)

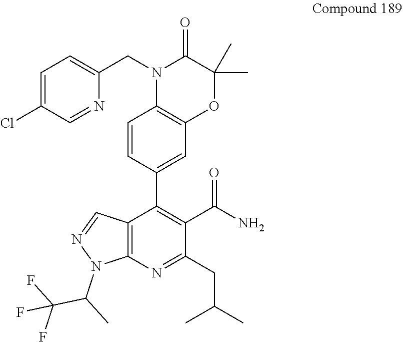 [SMILES: CC(C)Cc1nc2c(cnn2C(C)C(F)(F)F)c(-c2ccc3c(c2)OC(C)(C)C(=O)N3Cc2ccc(Cl)cn2)c1C(N)=O]

Compound 189 was synthesized following the similar route of Example 51, using 3,3,3-trifluoro-2-methylpropanoic acid in step A, and then following the similar route of Example 2, using 4-[(5-chloropyridin-2-yl)methyl]-2,2-dimethyl-3-oxo-3,4-dihydro-2H-benzo[b][1,4]oxazine-7-carbaldehyde and 1-(1,1,1-trifluoropropan-2-yl)-1H-pyrazol-5-amine in step B.

¹H NMR (400 MHz, DMSO-d₆) δ 8.59 (d, J=2.4 Hz, 1H), 8.06 (s, 1H), 7.94 (dd, J₁=2.4 Hz, J₂=8.4 Hz, 1H), 7.76 (m, 1H), 7.55 (m, 1H), 7.40 (d, J=8.4 Hz, 1H), 7.30 (d, J=2 Hz, 1H), 7.26 (dd, J₁=2 Hz, J₂=8.4 Hz, 1H), 7.14 (d, J=8 Hz, 1H), 5.80-5.84 (m, 1H), 5.25 (s, 2H), 2.74-2.87 (m, 2H), 2.32-2.37 (m, 1H), 1.81 (d, J=7.2 Hz, 3H), 1.51 (s, 6H), 0.94 (dd, J₁=2.8 Hz, J₂=6.8 Hz, 6H). ¹⁹F NMR (376 MHz, DMSO-d₆) δ −74.08. LC-MS: m/z 615.5 (M+H)⁺.

### 4-{3-[(5-cyano-2-pyridyl)methyl]-2-oxo-2,3-dihydro-1,3-benzoxazol-6-yl}-6-isobutyl-1-(2,2,2-trifluoro-1-methylethyl)-1,2,7-triaza-1H-indene-5-carboxamide (Compound 190)

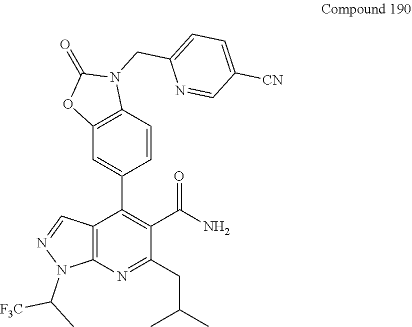 [SMILES: CC(C)Cc1nc2c(cnn2C(C)C(F)(F)F)c(-c2ccc3c(c2)oc(=O)n3Cc2ccc(C#N)cn2)c1C(N)=O]

Compound 190 was synthesized following the similar route of Example 26, using 1-(1,1,1-trifluoropropan-2-yl)-1H-pyrazol-5-amine in step A.

¹H NMR (400 MHz, DMSO-d₆) δ 8.98 (dd, J₁=0.8 Hz, J₂=2 Hz, 1H), 8.37 (dd, J₁=2 Hz, J₂=8 Hz, 1H), 8.09 (s, 1H), 7.79 (d, J=1.6 Hz, 1H), 7.77 (d, J=8.8 Hz, 1H), 7.64 (d, J=1.6 Hz, 1H), 7.55 (d, J=1.6 Hz, 1H), 7.46 (dd, J₁=1.6 Hz, J₂=8 Hz, 1H), 7.35 (d, J=8 Hz, 1H), 5.82-5.86 (m, 1H), 5.36 (s, 2H), 2.76-2.89 (m, 2H), 2.34-2.40 (m, 1H), 1.82 (d, J=7.2 Hz, 3H), 0.95 (dd, J₁=2 Hz, J₂=6.8 Hz, 6H). ¹⁹F NMR (376 MHz, DMSO-d₆) δ −74.08. LC-MS: m/z 564.4 (M+H)⁺.

### 4-{3-[(5-cyano-2-pyridyl)methyl]-2-oxo-2,3-dihydro-1,3-benzoxazol-6-yl}-6-(cyclopropylmethyl)-1-isopropyl-1,2,7-triaza-1H-indene-5-carboxamide (Compound 191)

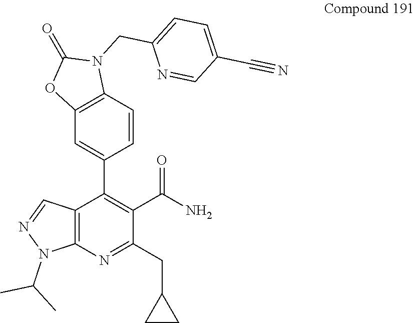 [SMILES: CC(C)n1ncc2c(-c3ccc4c(c3)oc(=O)n4Cc3ccc(C#N)cn3)c(C(N)=O)c(CC3CC3)nc21]

Compound 191 was synthesized following the similar route of Example 51, using 1-isopropyl-1H-pyrazol-5-amine, 4-cyclopropyl-3-oxobutanamide and 6-((6-formyl-2-oxobenzo[d]oxazol-3(2H)-yl)methyl) nicotinonitrile in step G.

¹H NMR (400 MHz, CD₃OD) δ 8.86 (d, J=1.2 Hz, 1H) 8.20 (dd, J=8.4, 2.0 Hz, 1H) 7.88 (s, 1H) 7.68 (d, J=8.0 Hz, 1H) 7.59 (d, J=1.2 Hz, 1H) 7.46 (dd, J=8.0, 1.6 Hz, 1H) 7.23 (d, J=8.0 Hz, 1H) 5.30-5.36 (m, 3H) 2.93 (d, J=7.2 Hz, 2H) 1.58 (d, J=6.4 Hz, 6H) 1.34-1.40 (m, 1H) 0.50-0.55 (m, 2H) 0.35 (d, J=5.6 Hz, 2H). LC-MS: m/z 659.9 (M+H)⁺.

### 4-{4-[(5-chloro-2-pyridyl)methyl]-2,2-dimethyl-3-oxo-3,4-dihydro-2H-1,4-benzoxazin-7-yl}-6-isobutyl-1-(2,2,2-trifluoroethyl)-1,2,7-triaza-1H-indene-5-carboxamide (Compound 192)

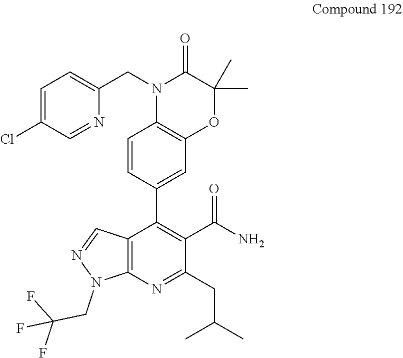 [SMILES: CC(C)Cc1nc2c(cnn2CC(F)(F)F)c(-c2ccc3c(c2)OC(C)(C)C(=O)N3Cc2ccc(Cl)cn2)c1C(N)=O]

Compound 192 was synthesized following the similar route of Example 46, using 2,2,2-trifluoroethyl trifluoromethanesulfonate in step A and then synthesized following the similar route of Example 51, using 4-[(5-chloropyridin-2-yl)methyl]-2,2-dimethyl-3-oxo-3,4-dihydro-2H-benzo[b][1,4]oxazine-7-carbaldehyde, 1-(2,2,2-trifluoroethyl)-1H-pyrazol-5-amine and tert-butyl(5-methyl-3-oxohexanoyl)carbamate in step G.

¹H NMR (400 MHz, CD₃OD) δ 8.52 (d, J=2.4 Hz, 1H), 7.99 (s, 1H), 7.83 (dd, J=8.4, 2.4 Hz, 1H), 7.35 (d, J=8.4 Hz, 1H), 7.32 (d, J=2.0 Hz, 1H), 7.26 (dd, J=8.4, 2.0 Hz, 1H), 7.14 (d, J=8.4 Hz, 1H), 5.30 (s, 2H), 5.23 (q, J=8.8 Hz, 2H), 2.92 (d, J=6.8 Hz, 2H), 2.42 (dt, J=13.6, 6.8 Hz, 1H), 1.58 (s, 6H), 0.99 (d, J=6.4 Hz, 6H). ¹⁹F NMR (376 MHz, CD₃OD) δ −72.37 LC-MS: m/z 601.2 (M+H)⁺.

### 4-{4-[(5-chloro-2-pyridyl)methyl]-2,2-dimethyl-3-oxo-3,4-dihydro-2H-1,4-benzoxazin-7-yl}-1-(2,2,2-trifluoroethyl)-6-(2,2,2-trifluoroethyl)-1,2,7-triaza-1H-indene-5-carboxamide (Compound 193)

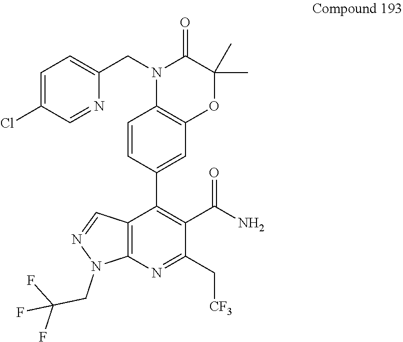 [SMILES: CC1(C)Oc2cc(-c3c(C(N)=O)c(CC(F)(F)F)nc4c3cnn4CC(F)(F)F)ccc2N(Cc2ccc(Cl)cn2)C1=O]

Compound 193 was synthesized following the similar route of Example 46, using 2,2,2-trifluoroethyl trifluoromethanesulfonate in step A and then synthesized following the similar route of Example 51, using 4-[(5-chloropyridin-2-yl)methyl]-2,2-dimethyl-3-oxo-3,4-dihydro-2H-benzo[b][1,4]oxazine-7-carbaldehyde, 1-(2,2,2-trifluoroethyl)-1H-pyrazol-5-amine and tert-butyl(5,5,5-trifluoro-3-oxopentanoyl)carbamate in step G.

¹H NMR (400 MHz, CD₃OD) § 8.51 (d, J=2.0 Hz, 1H), 8.09 (s, 1H), 7.83 (dd, J=8.4, 2.4 Hz, 1H), 7.36 (d, J=8.4 Hz, 1H), 7.32 (d, J=1.6 Hz, 1H), 7.25-7.29 (m, 1H), 7.17 (d, J=8.4 Hz, 1H), 5.30 (s, 2H), 5.21-5.28 (m, 2H), 3.98 (q, J=10.4 Hz, 2H), 1.58 (s, 6H). ¹⁹F NMR (376 MHz, CD₃OD) δ −64.42, −72.40. LC-MS: m/z 627.1 (M+H)⁺.

### 4-{4-[(5-chloro-2-pyridyl)methyl]-2,2-dimethyl-3-oxo-3,4-dihydro-2H-1,4-benzoxazin-7-yl}-1-(2,2,2-trifluoroethyl)-6-(3,3,3-trifluoro-2-methylpropyl)-1,2,7-triaza-1H-indene-5-carboxamide (Compound 194)

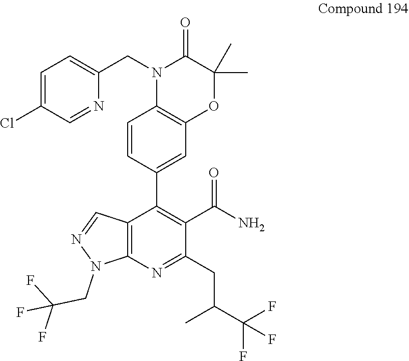 [SMILES: CC(Cc1nc2c(cnn2CC(F)(F)F)c(-c2ccc3c(c2)OC(C)(C)C(=O)N3Cc2ccc(Cl)cn2)c1C(N)=O)C(F)(F)F]

Compound 194 was synthesized following the similar route of Example 46, using 2,2,2-trifluoroethyl trifluoromethanesulfonate in step A and then synthesized following the similar route of Example 51, using 4-[(5-chloropyridin-2-yl)methyl]-2,2-dimethyl-3-oxo-3,4-dihydro-2H-benzo[b][1,4]oxazine-7-carbaldehyde, 1-(2,2,2-trifluoroethyl)-1H-pyrazol-5-amine and tert-butyl(6,6,6-trifluoro-5-methyl-3-oxohexanoyl)carbamate in step G.

¹H NMR (400 MHz, CD₃OD) § 8.51 (d, J=2.0 Hz, 1H), 8.07-8.01 (m, 1H), 7.83 (dd, J=2.0, 8.4 Hz, 1H), 7.39-7.31 (m, 2H), 7.30-7.24 (m, 1H), 7.16 (d, J=8.4 Hz, 1H), 5.31 (s, 2H), 5.29-5.20 (m, 2H), 3.44 (dd, J=3.2, 15.2 Hz, 1H), 3.30-3.21 (m, 1H), 3.01 (dd, J=10.4, 15.2 Hz, 1H), 1.58 (s, 6H), 1.14 (d, J=6.8 Hz, 3H). ¹⁹F NMR (376 MHz, CD₃OD) δ −72.35, −74.98. LC-MS: m/z 655.1 (M+H)⁺.

### Example 53

### 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(S)-1-methyl-2-trifluoromethoxyethyl]-1,2,7-triaza-1H-indene-5-carboxamide (Compound 195)

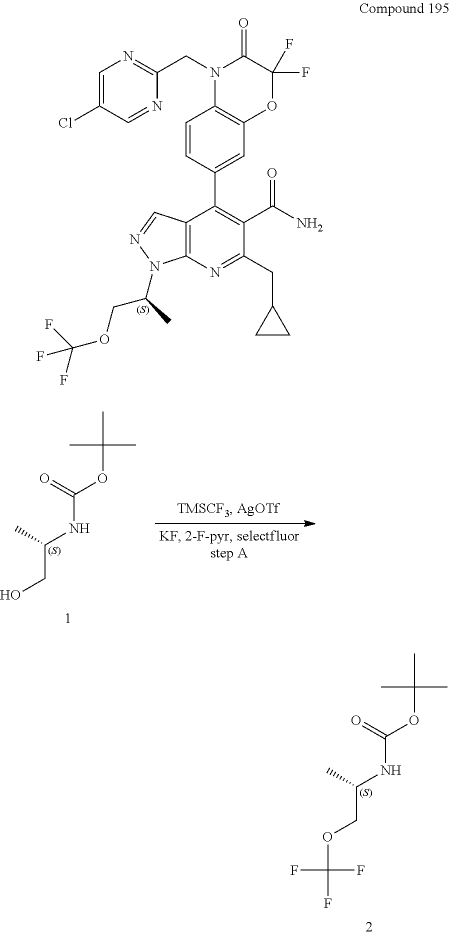

### Step A: tert-butyl(S)-(1-(trifluoromethoxy)propan-2-yl)carbamate

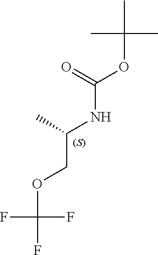

A mixture of tert-butyl(S)-(1-hydroxypropan-2-yl)carbamate (8 g, 45.66 mmol), 2-fluoropyridine (13.30 g, 136.97 mmol, 11.77 mL), AgOTf (35.19 g, 136.97 mmol), KF (10.61 g, 182.62 mmol), Select F (24.26 g, 68.48 mmol) and TMSCF3 (19.48 g, 136.97 mmol) in EtOAc (100 mL) was degassed and purged with N₂ for 3 times, then the mixture was stirred at 20° C. for 16 hr under N₂ atmosphere. The reaction mixture was filtered and concentrated under reduced pressure to give a residue, which was purified by flash silica gel chromatography (Eluent of 0~15% Ethyl acetate in Petroleum ether, gradient @ 40 mL/min) to give the product tert-butyl(S)-(1-(trifluoromethoxy)propan-2-yl)carbamate (4.6 g, 41.42%) as a colorless oil. ¹H NMR (400 MHz, CDCl₃) δ 4.59 (br s, 1H), 4.02-3.84 (m, 3H), 1.44 (s, 9H), 1.22 (d, J=6.8 Hz, 3H). ¹⁹F NMR (376 MHz, CDCl₃) δ −60.81.

### 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(S)-1-methyl-2-trifluoromethoxyethyl]-1,2,7-triaza-1H-indene-5-carboxamide (Compound 195)

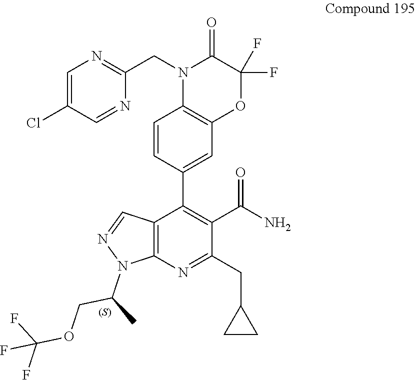

Compound 195 was then synthesized following the similar route of Example 51, using tert-butyl (S)-(1-(trifluoromethoxy)propan-2-yl)carbamate in step B.

¹H NMR (400 MHz, DMSO-d₆) δ 8.93 (s, 2H), 8.00 (s, 1H), 7.88 (d, J=2.0 Hz, 1H), 7.64 (d, J=2.0 Hz, 1H), 7.62 (d, J=1.6 Hz, 1H), 7.49 (dd, J₁=2.0 Hz, J₂=8.8 Hz, 1H), 7.44 (d, J=8.8 Hz, 1H), 5.51 (s, 2H), 5.36-5.41 (m, 1H), 4.64 (t, J=10.0 Hz, 1H), 4.54 (dd, J₁=4.0 Hz, J₂=10.0 Hz, 1H), 2.76-2.88 (m, 2H), 1.52 (d, J=6.8 Hz, 3H), 1.26-1.30 (m, 1H), 0.43-0.47 (m, 2H), 0.30-0.32 (m, 2H). ¹⁹F NMR (376 MHz, DMSO-d₆) δ −58.99, −74.86. LC-MS: m/z 652.1 (M+H)⁺.

### 6-(cyclopropylmethyl)-4-{2,2-difluoro-4-[(5-fluoro-2-pyrimidinyl)methyl]-3-oxo-3,4-dihydro-2H-1,4-benzoxazin-7-yl}-1-[(S)-1-methyl-2-trifluoromethoxyethyl]-1,2,7-triaza-1H-indene-5-carboxamide (compound 196)

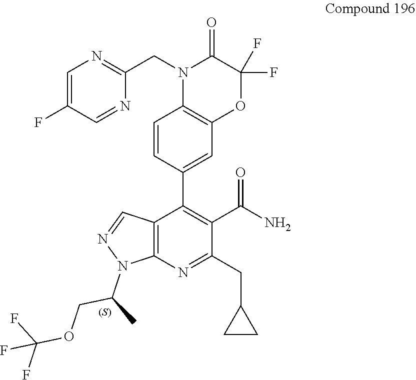

Compound 196 was synthesized following the similar route of Example 51, using 2,2-difluoro-4-((5-fluoropyrimidin-2-yl)methyl)-3-oxo-3,4-dihydro-2H-benzo[b][1,4]oxazine-7-carbaldehyde and tert-butyl(S)-(1-(1-(trifluoromethoxy)propan-2-yl)-1H-pyrazol-5-yl)carbamate in step G.

¹H NMR (400 MHz, DMSO-d₆) δ 8.94 (s, 2H), 7.99 (s, 1H), 7.88 (d, J=1.6 Hz, 1H), 7.64 (d, J=1.6 Hz, 1H), 7.61 (d, J=2.0 Hz, 1H), 7.49 (dd, J₁=1.6 Hz, J₂=8.4 Hz, 1H), 7.43 (d, J=8.8 Hz, 1H), 5.52 (s, 2H), 5.36-5.41 (m, 1H), 4.63 (t, J=10.0 Hz, 1H), 4.53 (dd, J₁=4.4 Hz, J₂=10.4 Hz, 1H), 2.75-2.88 (m, 2H), 1.52 (d, J=6.8 Hz, 3H), 1.26-1.31 (m, 1H), 0.43-0.47 (m, 2H), 0.30-0.31 (m, 2H). ¹⁹F NMR (376 MHz, DMSO-d₆) δ −58.99, −74.89, −139.28. LC-MS: m/z 636.5 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-methyl-2-trifluoromethoxyethyl]-1,2,7-triaza-1H-indene-5-carboxamide (Compound 197)

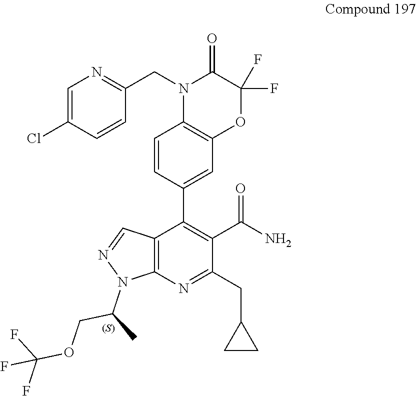

Compound 197 was synthesized following the similar route of Example 51, using 4-((5-chloropyridin-2-yl)methyl)-2,2-difluoro-3-oxo-3,4-dihydro-2H-benzo[b][1,4]oxazine-7-carbaldehyde and tert-butyl(S)-(1-(1-(trifluoromethoxy)propan-2-yl)-1H-pyrazol-5-yl)carbamate in step G.

¹H NMR (400 MHz, DMSO-d₆) δ 8.59 (d, J=2.4 Hz, 1H), 7.99-8.01 (m, 2H), 7.86 (d, J=1.6 Hz, 1H), 7.59-7.62 (m, 3H), 7.49 (dd, J₁=1.6 Hz, J₂=8.4 Hz, 1H), 7.45 (d, J=8.4 Hz, 1H), 5.43 (s, 2H), 5.36-5.41 (m, 1H), 4.63 (t, J=9.8 Hz, 1H), 4.53 (dd, J₁=4.4 Hz, J₂=10.4 Hz, 1H), 2.75-2.87 (m, 2H), 1.52 (d, J=6.8 Hz, 3H), 1.26-1.30 (m, 1H), 0.43-0.46 (m, 2H), 0.28-0.31 (m, 2H). ¹⁹F NMR (376 MHz, DMSO-d₆) δ −58.99, −74.43. LC-MS: m/z 651.4 (M+H)⁺.

### 6-(cyclopropylmethyl)-4-{2,2-difluoro-4-[(5-fluoro-2-pyridyl)methyl]-3-oxo-3,4-dihydro-2H-1,4-benzoxazin-7-yl}-1-[(S)-1-methyl-2-trifluoromethoxyethyl]-1,2,7-triaza-1H-indene-5-carboxamide (Compound 198)

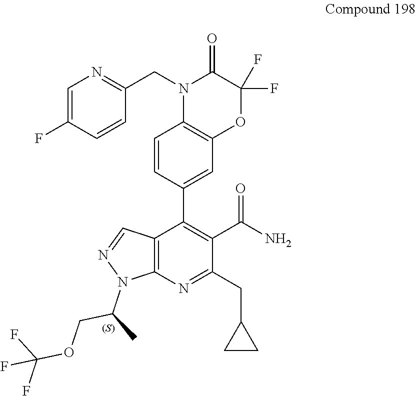

Compound 198 was synthesized following the similar route of Example 51, using 2,2-difluoro-4-((5-fluoropyridin-2-yl)methyl)-3-oxo-3,4-dihydro-2H-benzo[b][1,4]oxazine-7-carbaldehyde and tert-butyl(S)-(1-(1-(trifluoromethoxy)propan-2-yl)-1H-pyrazol-5-yl)carbamate in step G.

¹H NMR (400 MHz, DMSO-d₆) δ 8.54 (d, J=2.8 Hz, 1H), 7.98 (s, 1H), 7.86 (d, J=2.0 Hz, 1H), 7.78-7.83 (m, 1H), 7.59-7.65 (m, 3H), 7.45-7.51 (m, 2H), 5.42 (s, 2H), 5.36-5.41 (m, 1H), 4.63 (t, J=9.8 Hz, 1H), 4.54 (dd, J₁=4 Hz, J₂=10 Hz, 1H), 2.75-2.87 (m, 2H), 1.52 (d, J=6.8 Hz, 3H), 1.26-1.30 (m, 1H), 0.43-0.47 (m, 2H), 0.28-0.31 (m, 2H). ¹⁹F NMR (376 MHz, DMSO-d₆) δ −58.99, −74.47, −128.82. LC-MS: m/z 636.0 (M+H)⁺.

### 4-{4-[(5-chloro-2-pyridyl)methyl]-2,2-dimethyl-3-oxo-3,4-dihydro-2H-1,4-benzoxazin-7-yl}-6-(cyclopropylmethyl)-1-[(S)-1-methyl-2-trifluoromethoxyethyl]-1,2,7-triaza-1H-indene-5-carboxamide (Compound 199)

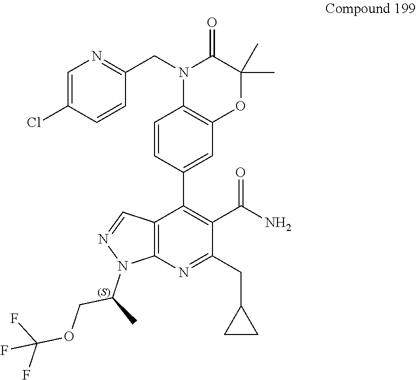

Compound 199 was synthesized following the similar route of Example 51, using 4-((5-chloropyridin-2-yl)methyl)-2,2-dimethyl-3-oxo-3,4-dihydro-2H-benzo[b][1,4]oxazine-7-carbaldehyde and tert-butyl(S)-(1-(1-(trifluoromethoxy)propan-2-yl)-1H-pyrazol-5-yl)carbamate in step G.

¹H NMR (400 MHz, CD₃OD) § 8.53 (d, J=2.4 Hz, 1H), 7.95 (s, 1H), 7.85 (dd, J=2.4, 8.4 Hz, 1H), 7.37 (d, J=8.4 Hz, 1H), 7.33 (d, J=2.0 Hz, 1H), 7.28 (dd, J=2.0, 8.4 Hz, 1H), 7.16 (d, J=8.4 Hz, 1H), 5.53-5.43 (m, 1H), 5.32 (s, 2H), 4.62 (t, J=9.6 Hz, 1H), 4.43 (dd, J=4.4, 10.0 Hz, 1H), 3.01-2.86 (m, 2H), 1.63 (d, J=6.8 Hz, 3H), 1.60 (s, 6H), 1.41-1.32 (m, 1H), 0.57-0.50 (m, 2H), 0.39-0.33 (m, 2H). ¹⁹F NMR (376 MHz, CD₃OD) δ −62.39. LC-MS: m/z 643.3 (M+H)⁺.

### Example 54

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[1-(difluoromethoxymethyl)cyclopropyl]-1,2,7-triaza-1H-indene-5-carboxamide (Compound 200)

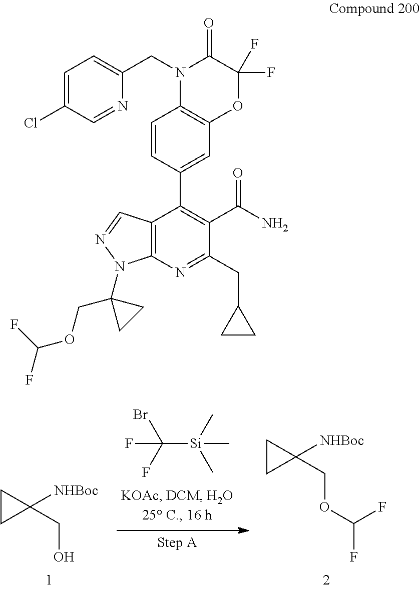

### Step A: tert-butyl(1-((difluoromethoxy)methyl)cyclopropyl)carbamate

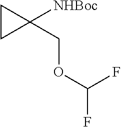 [SMILES: CC(C)(C)OC(=O)NC1(COC(F)F)CC1]

To a solution of 2-methylpropan-2-yl {[(hydroxymethyl)cyclopropyl]amino}methanoate (1000 mg, 5.341 mmol) in DCM (20 mL) and H₂O (20 mL) were added (bromodifluoromethyl)trimethylsilane (4.153 mL, 26.704 mmol) and KOAc (2620.70 mg, 26.704 mmol). The reaction mixture was stirred at 25° C. for 16 hours under N₂. The mixture was added to water (50 mL), extracted with EA (50 mL×2), the combined organic layers were washed with brine (50 mL×3), dried over sodium sulfate, filtered and concentrated under vacuum. The resulting residue was purified by silica gel chromatography (eluting PE/EA=20/1) to afford tert-butyl(1-((difluoromethoxy)methyl)cyclopropyl)carbamate (400 mg, 31.57%) as a white solid. ¹H NMR (400 MHz, DMSO-d₆) 8.26 (s, 1H), 6.23 (t, J=149.6 Hz, 1H), 3.85 (s, 2H), 1.44 (s, 9H), 0.79-0.90 (m, 4H).

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[1-(difluoromethoxymethyl)cyclopropyl]-1,2,7-triaza-1H-indene-5-carboxamide (Compound 200)

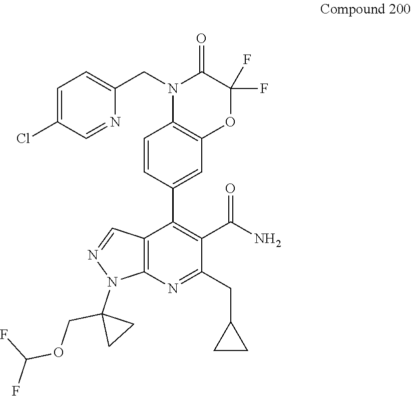 [SMILES: NC(=O)c1c(CC2CC2)nc2c(cnn2C2(COC(F)F)CC2)c1-c1ccc2c(c1)OC(F)(F)C(=O)N2Cc1ccc(Cl)cn1]

Compound 200 was then synthesized following the similar route of Example 51, using tert-butyl(1-((difluoromethoxy)methyl)cyclopropyl)carbamate in step B and then using 4-((5-chloropyridin-2-yl)methyl)-2,2-difluoro-3-oxo-3,4-dihydro-2H-benzo[b][1,4]oxazine-7-carbaldehyde in step G.

¹H NMR (400 MHz, DMSO-d₆) δ 8.59 (d, J=2.4 Hz, 1H), 8.00 (dd, J₁=2.4 Hz, J₂=8.4 Hz, 1H), 7.90 (s, 1H), 7.85 (d, J=1.6 Hz, 1H), 7.58-7.60 (m, 3H), 7.42-7.48 (m, 2H), 6.62 (t, J=75.6 Hz, 1H), 5.42 (s, 2H), 4.28 (s, 2H), 2.81 (d, J=7.2 Hz, 2H), 1.42-1.43 (m, 2H), 1.35-1.36 (m, 2H), 1.23-1.30 (m, 1H), 0.45-0.47 (m, 2H), 0.31-0.32 (m, 2H). ¹⁹F NMR (376 MHz, DMSO-d₆) δ −74.41, −82.89. LC-MS: m/z 645.8 (M+H)⁺.

### 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[1-(difluoromethoxymethyl)cyclopropyl]-1,2,7-triaza-1H-indene-5-carboxamide (compound 201)

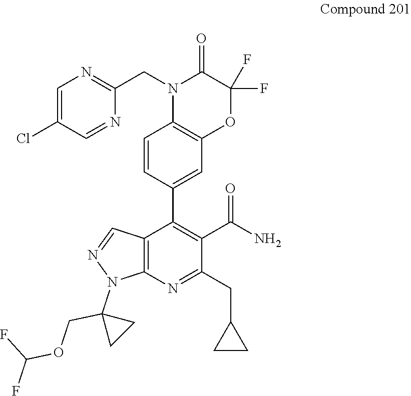 [SMILES: NC(=O)c1c(CC2CC2)nc2c(cnn2C2(COC(F)F)CC2)c1-c1ccc2c(c1)OC(F)(F)C(=O)N2Cc1ncc(Cl)cn1]

Compound 201 was synthesized following the similar route of Example 51, using tert-butyl(1-((difluoromethoxy)methyl)cyclopropyl)carbamate in step B.

¹H NMR (400 MHz, DMSO-d₆) δ 8.98 (s, 2H), 7.91 (s, 1H), 7.87 (d. J=2.0 Hz, 1H), 7.61 (d, J=1.6 Hz, 2H), 7.42-7.48 (m, 2H), 6.63 (t, J=75.8 Hz, 1H), 5.51 (s, 2H), 4.28 (s, 2H), 2.82 (d, J=7.2 Hz, 2H), 1.40-1.45 (m, 2H), 1.33-1.37 (m, 2H), 1.26-1.30 (m, 1H), 0.44-0.49 (m, 2H), 0.30-0.34 (m, 2H). ¹⁹F NMR (376 MHz, DMSO-d₆) δ −74.85, −82.88. LC-MS: m/z 646.4 (M+H)⁺.

### Example 56

### 6-(cyclopropylmethyl)-4-{2,2-difluoro-4-[(5-fluoro-2-pyrimidinyl)methyl]-3-oxo-3,4-dihydro-2H-1,4-benzoxazin-7-yl}-1-[1-(5-fluoro-2-pyridyl)cyclopropyl]-1,2,7-triaza-1H-indene-5-carboxamide (Compound 207)

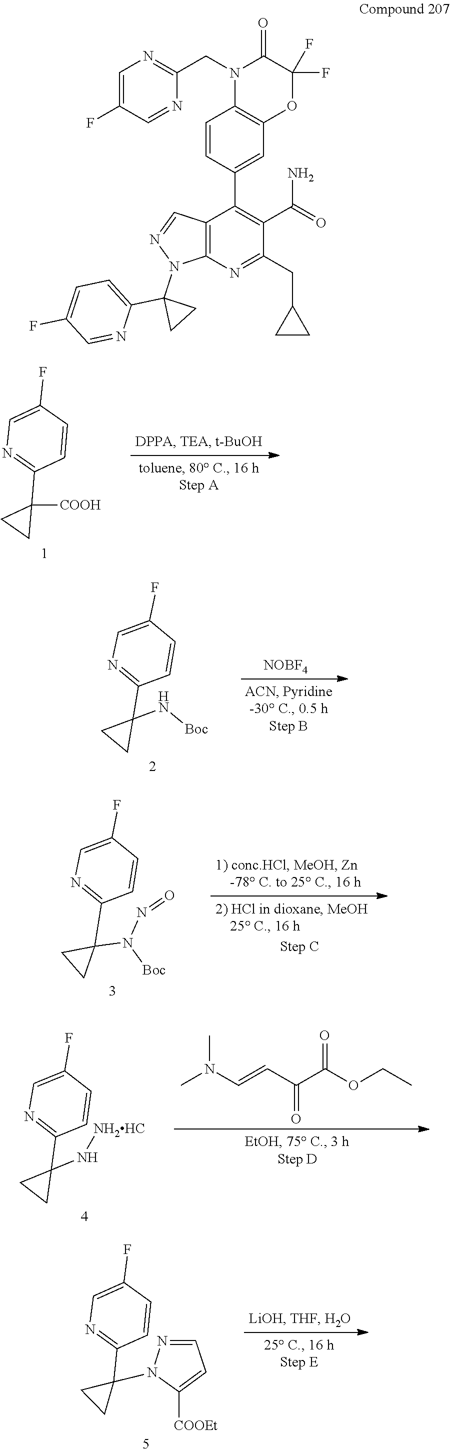

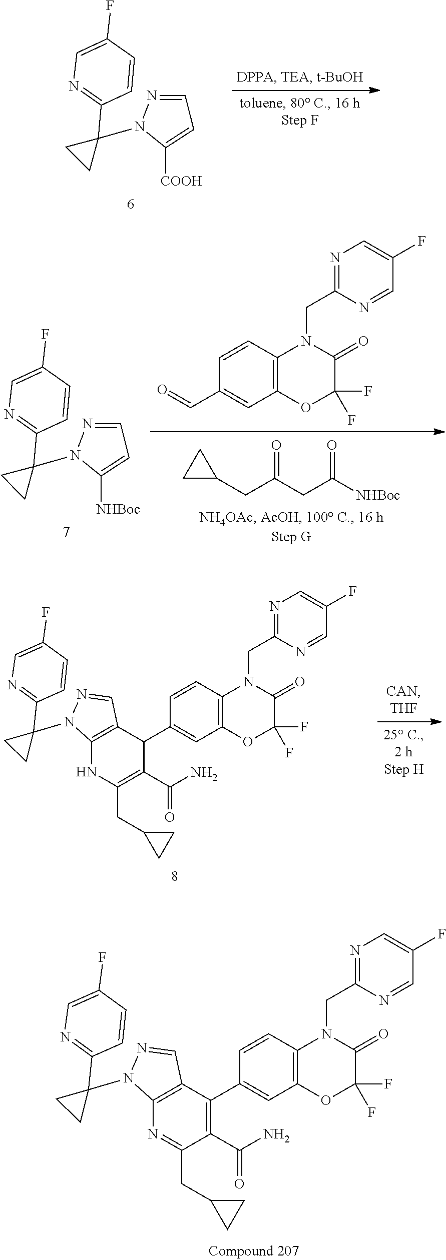

### Step A: tert-butyl(1-(5-fluoropyridin-2-yl)cyclopropyl)carbamate

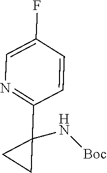 [SMILES: CC(C)(C)OC(=O)NC1(c2ccc(F)cn2)CC1]

To a solution of 1-(5-fluoropyridin-2-yl)cyclopropane-1-carboxylic acid (5 g, 27.598 mmol) in toluene (100 mL) and t-BuOH (100 mL) were added DPPA (11.38 g, 41.398 mmol) and TEA (4.19 g, 41.398 mmol), the mixture was stirred at 80° C. for 16 h under N₂. The reaction mixture was concentrated under vacuum. The resulting residue was purified by silica gel column chromatography (eluting PE/EA=100/1) to afford tert-butyl(1-(5-fluoropyridin-2-yl)cyclopropyl)carbamate (5.3 g, 76.12%) as a yellow oil. LC-MS: m/z 253.2 (M+H)⁺.

### Step B: tert-butyl(1-(5-fluoropyridin-2-yl)cyclopropyl)(nitroso)carbamate

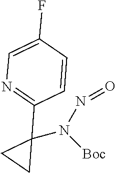 [SMILES: CC(C)(C)OC(=O)N(N=O)C1(c2ccc(F)cn2)CC1]

To a solution of tert-butyl(1-(5-fluoropyridin-2-yl)cyclopropyl)carbamate (5.3 g, 21.008 mmol) in ACN (200 mL) was added Pyridine (3.398 mL, 42.015 mmol). The mixture was cooled to −30° C., NOBF₄ (5.00 g, 42.015 mmol) was added. The reaction mixture was stirred at −30° C. for 30 mins under N₂. The reaction mixture was poured into H₂O (50 mL) and extracted with EA (50 mL×2). The combined organics was washed brine (20 mL) and dried over sodium sulfate, filtered and concentrated under vacuum. The resulting residue was purified by silica gel column chromatography (eluting PE/EA=80/1) to afford tert-butyl(1-(5-fluoropyridin-2-yl)cyclopropyl)(nitroso)carbamate (5.4 g, 91.38%) as a yellow oil. ¹H NMR (400 MHz, DMSO-d₆) δ 8.37 (d, J=2.8 Hz, 1H), 7.58 (t, J=4.4 Hz, 1H), 6.87 (dd, J₁=4.0 Hz, J₂=8.8 Hz, 1H), 1.55 (s, 9H), 1.17 (m, 4H).

### Step C: 5-fluoro-2-(1-hydrazineylcyclopropyl)pyridine hydrochloride

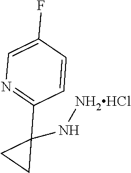

The solution of tert-butyl(1-(5-fluoropyridin-2-yl)cyclopropyl)(nitroso)carbamate (5.4 g, 19.197 mmol) in MeOH (200 mL) was cooled to −78° C. conc. HCl (15.998 mL, 191.973 mmol) was added, followed by Zn (12.55 g, 191.973 mmol). The mixture was allowed to warm to 25° C. and stirred at 25° C. for 16 h under N₂. The mixture was filtered and concentrated to afford a crude product. To the crude product was added MeOH (100 mL), followed by HCl in 1,4-dioxane (4M, 29.930 mL, 119.72 mmol). The reaction mixture was stirred at 25° C. for 16 h. The mixture was concentrated under vacuum to afford 5-fluoro-2-(1-hydrazineylcyclopropyl)pyridine hydrochloride (2 g, 62.31%) as a yellow oil, which was used in the next step without further purification. LC-MS: m/z 168.2 (M+H-HCl)⁺.

### Step D: ethyl 1-(1-(5-fluoropyridin-2-yl)cyclopropyl)-1H-pyrazole-5-carboxylate

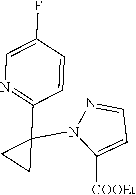 [SMILES: CCOC(=O)c1ccnn1C1(c2ccc(F)cn2)CC1]

To a solution of 5-fluoro-2-(1-hydrazineylcyclopropyl)pyridine (2 g, 11.962 mmol) in EtOH (200 mL) was added ethyl(3E)-4-(dimethylamino)-2-oxobut-3-enoate (4.10 g, 23.925 mmol), the reaction mixture was stirred at 75° C. for 3 h under N₂. The reaction mixture was concentrated under vacuum. The resulting residue was purified by silica gel column chromatography (eluting PE/EA=40/1) to afford ethyl 1-(1-(5-fluoropyridin-2-yl)cyclopropyl)-1H-pyrazole-5-carboxylate (2 g, 60.73%) as a yellow oil. LC-MS: m/z 276.2 (M+H)⁺.

### Step E: 1-(1-(5-fluoropyridin-2-yl)cyclopropyl)-1H-pyrazole-5-carboxylic acid

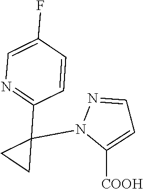 [SMILES: O=C(O)c1ccnn1C1(c2ccc(F)cn2)CC1]

To a solution of ethyl 1-(1-(5-fluoropyridin-2-yl)cyclopropyl)-1H-pyrazole-5-carboxylate (1.32 g, 4.795 mmol) in THF (50 mL) and H₂O (50 mL) was added LiOH (2.01 g, 47.951 mmol), the mixture was stirred at 25° C. for 16 h. The mixture was poured into H₂O (50 mL), extracted with EA (50 mL×2). The combined organics were washed with brine (20 mL) and dried over sodium sulfate, filtered and concentrated under vacuum. The resulting residue was concentrated to afford 1-(1-(5-fluoropyridin-2-yl)cyclopropyl)-1H-pyrazole-5-carboxylic acid (1.29 g, 100%) as a yellow oil, which was used in the next step without further purification. LC-MS: m/z 248.2 (M+H)⁺.

### Step F: tert-butyl(1-(1-(5-fluoropyridin-2-yl)cyclopropyl)-1H-pyrazol-5-yl)carbamate

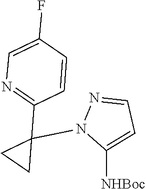 [SMILES: CC(C)(C)OC(=O)Nc1ccnn1C1(c2ccc(F)cn2)CC1]

To a solution of 1-(1-(5-fluoropyridin-2-yl)cyclopropyl)-1H-pyrazole-5-carboxylic acid (1.29 g, 5.218 mmol) in toluene (30 mL) and t-BuOH (30 mL) were added DPPA (2.15 g, 7.827 mmol) and TEA (1.088 mL, 7.827 mmol), the mixture was stirred at 80° C. for 16 h under N₂. The reaction mixture was concentrated under vacuum. The resulting residue was purified by silica gel column chromatography (eluting PE/EA=5/1) to afford tert-butyl(1-(1-(5-fluoropyridin-2-yl)cyclopropyl)-1H-pyrazol-5-yl) carbamate (1.2 g, 72.24%) as a yellow solid. ¹H NMR (400 MHz, DMSO-d₆) δ 8.97 (s, 1H), 8.46 (d, J=2.0 Hz, 1H), 7.53-7.58 (m, 1H), 7.50 (d, J=2.0 Hz, 1H), 6.22-6.25 (m, 2H), 1.72-1.75 (m, 2H), 1.63-1.66 (m, 2H), 1.35 (s, 9H). LC-MS: m/z 319.3 (M+H)⁺.

### Step G: 6-(cyclopropylmethyl)-4-(2,2-difluoro-4-((5-fluoropyrimidin-2-yl)methyl)-3-oxo-3,4-dihydro-2H-benzo[b][1,4]oxazin-7-yl)-1-(1-(5-fluoropyridin-2-yl)cyclopropyl)-4,7-dihydro-1H-pyrazolo[3,4-b]pyridine-5-carboxamide

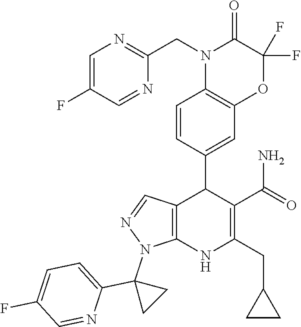 [SMILES: NC(=O)C1=C(CC2CC2)Nc2c(cnn2C2(c3ccc(F)cn3)CC2)C1c1ccc2c(c1)OC(F)(F)C(=O)N2Cc1ncc(F)cn1]

A mixture of 2,2-difluoro-4-((5-fluoropyrimidin-2-yl)methyl)-3-oxo-3,4-dihydro-2H-benzo[b][1,4]oxazine-7-carbaldehyde (11 g, 34.031 mmol), tert-butyl(1-(1-(5-fluoropyridin-2-yl)cyclopropyl)-1H-pyrazol-5-yl)carbamate (11 g, 34.553 mmol), tert-butyl(4-cyclopropyl-3-oxobutanoyl) carbamate (8.21 g, 34.031 mmol) and NH₄OAc (5.25 g, 68.063 mmol) in AcOH (160 mL) was stirred at 100° C. for 16 h under N₂. The reaction mixture was concentrated under vacuum to afford 6-(cyclopropylmethyl)-4-(2,2-difluoro-4-((5-fluoropyrimidin-2-yl)methyl)-3-oxo-3,4-dihydro-2H-benzo[b][1,4]oxazin-7-yl)-1-(1-(5-fluoropyridin-2-yl)cyclopropyl)-4,7-dihydro-1H-pyrazolo[3,4-b]pyridine-5-carboxamide (22 g, 100%) as a yellow oil, which was used in the next step without further purification. LC-MS: m/z 647.5 (M+H)⁺.

### Step H: 6-(cyclopropylmethyl)-4-{2,2-difluoro-4-[(5-fluoro-2-pyrimidinyl)methyl]-3-oxo-3,4-dihydro-2H-1,4-benzoxazin-7-yl}-1-[1-(5-fluoro-2-pyridyl)cyclopropyl]-1,2,7-triaza-1H-indene-5-carboxamide (Compound 207)

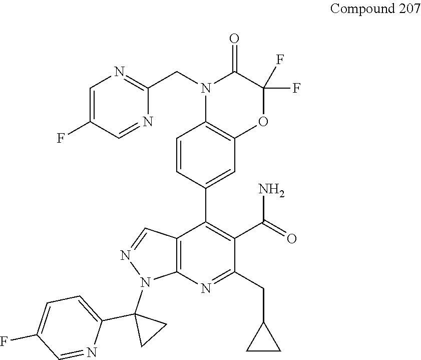 [SMILES: NC(=O)c1c(CC2CC2)nc2c(cnn2C2(c3ccc(F)cn3)CC2)c1-c1ccc2c(c1)OC(F)(F)C(=O)N2Cc1ncc(F)cn1]

A mixture of 6-(cyclopropylmethyl)-4-(2,2-difluoro-4-((5-fluoropyrimidin-2-yl)methyl)-3-oxo-3,4-dihydro-2H-benzo[b][1,4]oxazin-7-yl)-1-(1-(5-fluoropyridin-2-yl)cyclopropyl)-4,7-dihydro-1H-pyrazolo[3,4-b]pyridine-5-carboxamide (22 g, 34.024 mmol) and CAN (37.29 g, 68.047 mmol) in THF (150 mL) was stirred at 25° C. for 2 h. The reaction mixture was added to EA (200 mL) and aq. NaHCO₃ (200 mL), the mixture was stirred at rt for 30 min. The organic layer was separated and washed with brine (100 mL), dried over sodium sulfate, filtered and concentrated under vacuum. The resulting residue was purified by silica gel chromatography (eluting PE/EA=1/1) to give a crude. The crude (12 g, 75% purity) was added to EA (36 mL) and the mixture was stirred at rt for another 30 min, PE (180 mL) was added, the mixture was filtered, the cake was dried under vacuum at 50° C. for 30 min to afford a crude product. The crude product (9.2 g, 92% purity) was added to EtOH (90 mL), the mixture was stirred at 80° C. for 30 min, the mixture was allowed to cooled to rt slowly and stirred at rt for 16 h. The mixture was filtered, the cake was washed with PE (20 mL), dried under vacuum at 60° C. for 16 h to afford 6-(cyclopropylmethyl)-4-{2,2-difluoro-4-[(5-fluoro-2-pyrimidinyl)methyl]-3-oxo-3,4-dihydro-2H-1,4-benzoxazin-7-yl}-1-[1-(5-fluoro-2-pyridyl)cyclopropyl]-1,2,7-triaza-1H-indene-5-carboxamide (8.0 g, 35.94%). ¹H NMR (400 MHz, DMSO-d₆) δ 8.94 (s, 2H), 8.51 (d, J=2.8 Hz, 1H), 8.08 (s, 1H), 7.90 (d, J=1.6 Hz, 1H), 7.67 (d, J=2.0 Hz, 1H), 7.63 (d, J=1.6 Hz, 1H), 7.49-7.54 (m, 2H), 7.45 (d, J=8.4 Hz, 1H), 6.35 (dd, J₁=4.0 Hz, J₂=8.8 Hz, 1H), 5.53 (s, 2H), 2.75 (d, J=7.2 Hz, 2H), 1.93-1.96 (m, 2H), 1.80-1.84 (m, 2H), 1.14-1.21 (m, 1H), 0.34-0.39 (m, 2H), 0.20-0.24 (m, 2H). ¹⁹F NMR (376 MHz, DMSO-d₆) δ −74.90, −131.50, −139.26. LC-MS: m/z 645.4 (M+H)⁺.

### 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[1-(5-fluoro-2-pyridyl)cyclopropyl]-1,2,7-triaza-1H-indene-5-carboxamide (Compound 208)

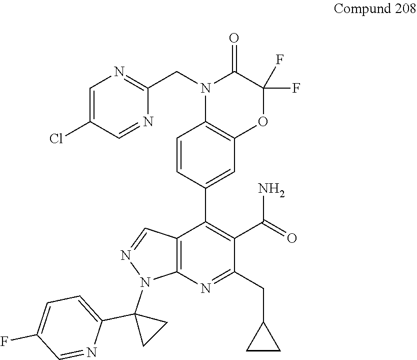 [SMILES: NC(=O)c1c(CC2CC2)nc2c(cnn2C2(c3ccc(F)cn3)CC2)c1-c1ccc2c(c1)OC(F)(F)C(=O)N2Cc1ncc(Cl)cn1]

Compound 208 was synthesized following the similar route of Example 56, using 4-((5-chloropyrimidin-2-yl)methyl)-2,2-difluoro-3-oxo-3,4-dihydro-2H-benzo[b][1,4]oxazine-7-carbaldehyde in step G.

¹H NMR (400 MHz, DMSO-d₆) δ 8.98 (s, 2H), 8.51 (d, J=3.2 Hz, 1H), 8.08 (s, 1H), 7.91 (d, J=1.6 Hz, 1H), 7.68 (d, J=2.0 Hz, 1H), 7.64 (d, J=1.6 Hz, 1H), 7.49-7.54 (m, 2H), 7.46 (d, J=8.8 Hz, 1H), 6.33-6.36 (m, 1H), 5.52 (s, 2H), 2.75 (d, J=7.2 Hz, 2H), 1.93-1.96 (m, 2H), 1.81-1.84 (m, 2H), 1.14-1.20 (m, 1H), 0.34-0.39 (m, 2H), 0.20-0.24 (m, 2H). ¹⁹F NMR (376 MHz, DMSO-d₆) δ −74.86, −131.49. LC-MS: m/z 661.4 (M+H)⁺.

### 6-(cyclopropylmethyl)-4-{2,2-difluoro-4-[(5-fluoro-2-pyridyl)methyl]-3-oxo-3,4-dihydro-2H-1,4-benzoxazin-7-yl}-1-[1-(5-fluoro-2-pyridyl)cyclopropyl]-1,2,7-triaza-1H-indene-5-carboxamide (Compound 209)

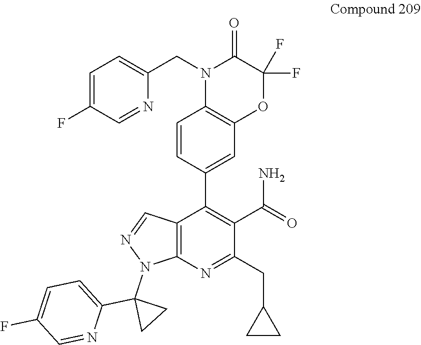 [SMILES: NC(=O)c1c(CC2CC2)nc2c(cnn2C2(c3ccc(F)cn3)CC2)c1-c1ccc2c(c1)OC(F)(F)C(=O)N2Cc1ccc(F)cn1]

Compound 209 was synthesized following the similar route of Example 56, using 4-((5-chloropyridin-2-yl)methyl)-2,2-difluoro-3-oxo-3,4-dihydro-2H-benzo[b][1,4]oxazine-7-carbaldehyde in step G.

¹H NMR (400 MHz, DMSO-d₆) δ 8.54 (d, J=3.2 Hz, 1H), 8.51 (d, J=2.8 Hz, 1H), 8.07 (s, 1H), 7.89 (d, J=1.6 Hz, 1H), 7.79-7.84 (m, 1H), 7.62-7.66 (m, 3H), 7.53 (dd, J₁=2 Hz, J₂=8.4 Hz, 2H), 7.48 (d, J=8.8 Hz, 1H), 6.32-6.35 (m, 1H), 5.43 (s, 2H), 2.74 (d, J=7.2 Hz, 2H), 1.93-1.96 (m, 2H), 1.80-1.83 (m, 2H), 1.13-1.21 (m, 1H), 0.34-0.39 (m, 2H), 0.20-0.24 (m, 2H). ¹⁹F NMR (376 MHz, DMSO-d₆) δ −74.49, −128.81, −131.49. LC-MS: m/z 644.4 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[1-(5-fluoro-2-pyridyl)cyclopropyl]-1,2,7-triaza-1H-indene-5-carboxamide (Compound 210)

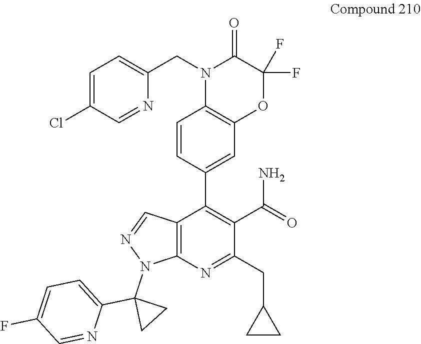 [SMILES: NC(=O)c1c(CC2CC2)nc2c(cnn2C2(c3ccc(F)cn3)CC2)c1-c1ccc2c(c1)OC(F)(F)C(=O)N2Cc1ccc(Cl)cn1]

Compound 210 was synthesized following the similar route of Example 56, using 4-((5-chloropyridin-2-yl)methyl)-2,2-difluoro-3-oxo-3,4-dihydro-2H-benzo[b][1,4]oxazine-7-carbaldehyde in step G.

¹H NMR (400 MHz, DMSO-d₆) δ 8.60 (m, 1H), 8.51 (d, J=2.0 Hz, 1H), 8.07 (s, 1H), 8.01 (dd, J₁=1.6 Hz, J₂=8.0 Hz, 1H), 7.89 (s, 1H), 7.59-7.65 (m, 3H), 7.45-7.53 (m, 3H), 6.33 (dd, J₁=4.4 Hz, J₂=8.8 Hz, 1H), 5.44 (s, 2H), 2.75 (d, J=6.8 Hz, 2H), 1.94 (m, 2H), 1.82 (m, 2H), 1.16 (m, 1H), 0.35-0.37 (m, 2H), 0.22-0.23 (m, 2H). ¹⁹F NMR (376 MHz, DMSO-d₆) δ −74.44, −131.49. LC-MS: m/z 660.7 (M+H)⁺.

### 6-(cyclopropylmethyl)-4-{2,2-difluoro-4-[(5-fluoro-3-methoxy-2-pyridyl)methyl]-3-oxo-3,4-dihydro-2H-1,4-benzoxazin-7-yl}-1-[1-(5-fluoro-2-pyridyl)cyclopropyl]-1,2,7-triaza-1H-indene-5-carboxamide (Compound 297)

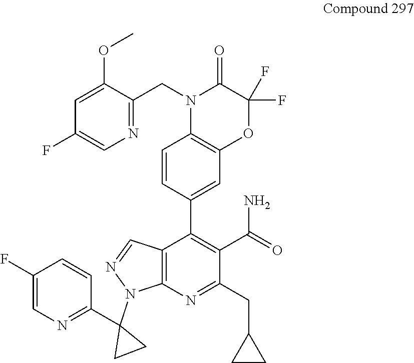 [SMILES: COc1cc(F)cnc1CN1C(=O)C(F)(F)Oc2cc(-c3c(C(N)=O)c(CC4CC4)nc4c3cnn4C3(c4ccc(F)cn4)CC3)ccc21]

Compound 297 was synthesized following the similar route of Example 56, using 4-((5-chloropyrimidin-2-yl)methyl)-2-fluoro-3-oxo-3,4-dihydro-2H-benzo[b][1,4]oxazine-7-carbaldehyde in step A.

¹H NMR (400 MHz, CD₃OD) δ 8.36 (d, J=2.8 Hz, 1H), 8.03 (s, 1H), 7.95 (d, J=2.4 Hz, 1H), 7.59 (d, J=2.0 Hz, 1H), 7.46 (dd, J=8.4, 2.0 Hz, 1H), 7.39 (dd, J=10.4, 2.4 Hz, 1H), 7.30 (td, J=8.8, 2.8 Hz, 1H), 7.24 (d, J=8.8 Hz, 1H), 6.35 (dd, J=8.8, 4.0 Hz, 1H), 5.41 (s, 2H), 3.99 (s, 3H), 2.86 (d, J=6.8 Hz, 2H), 2.00-2.06 (m, 2H), 1.85-1.92 (m, 2H), 1.19-1.27 (m, 1H), 0.38-0.46 (m, 2H), 0.22-0.29 (m, 2H). ¹⁹F NMR (376 MHz, CD₃OD) δ −77.81, −128.40, −133.57. LC-MS: m/z 674.3 (M+H)⁺.

### 6-(cyclopropylmethyl)-4-{2,2-difluoro-4-[(5-fluoro-4-methoxy-2-pyrimidinyl)methyl]-3-oxo-3,4-dihydro-2H-1,4-benzoxazin-7-yl}-1-[1-(5-fluoro-2-pyridyl)cyclopropyl]-1,2,7-triaza-1H-indene-5-carboxamide (Compound 298)

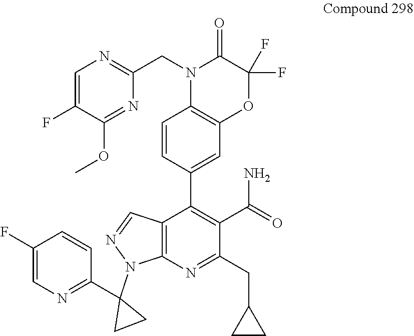 [SMILES: COc1nc(CN2C(=O)C(F)(F)Oc3cc(-c4c(C(N)=O)c(CC5CC5)nc5c4cnn5C4(c5ccc(F)cn5)CC4)ccc32)ncc1F]

Compound 298 was synthesized following the similar route of Example 56, using 4-((5-chloropyrimidin-2-yl)methyl)-2-fluoro-3-oxo-3,4-dihydro-2H-benzo[b][1,4]oxazine-7-carbaldehyde in step A.

¹H NMR (400 MHz, DMSO-d₆) δ 8.38 (d, J=2.8 Hz, 1H), 8.27 (d, J=2.4 Hz, 1H), 7.84 (s, 1H), 7.66 (s, 1H), 7.44 (s, 1H), 7.39 (s, 1H), 7.29-7.31 (m, 2H), 7.27 (d, J=8.8 Hz, 1H), 6.11 (dd, J₁=4.4 Hz, J₂=8.8 Hz, 1H), 5.17 (s, 2H), 3.70 (s, 3H), 2.51 (d, J=6.4 Hz, 2H), 1.71-1.73 (m, 2H), 1.57-1.59 (m, 2H), 0.91-0.95 (m, 1H), 0.13 (d, J=7.2 Hz, 2H), −0.01 (d, J=4 Hz, 2H). ¹⁹F NMR (376 MHz, DMSO-d₆) δ −75.34, −131.48, −156.01. LC-MS: m/z 675.3 (M+H)⁺.

### 6-(cyclopropylmethyl)-4-{2,2-difluoro-4-[1-(5-fluoro-2-pyrimidinyl)ethyl]-3-oxo-3,4-dihydro-2H-1,4-benzoxazin-7-yl}-1-[1-(5-fluoro-2-pyridyl)cyclopropyl]-1,2,7-triaza-1H-indene-5-carboxamide (Compound 299)

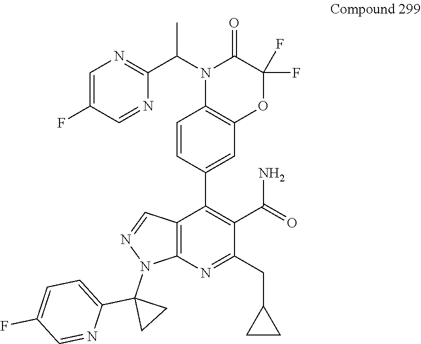 [SMILES: CC(c1ncc(F)cn1)N1C(=O)C(F)(F)Oc2cc(-c3c(C(N)=O)c(CC4CC4)nc4c3cnn4C3(c4ccc(F)cn4)CC3)ccc21]

Compound 299 was synthesized following the similar route of Example 56, using 2,2-difluoro-4-(1-(5-fluoropyrimidin-2-yl)ethyl)-3-oxo-3,4-dihydro-2H-benzo[b][1,4]oxazine-7-carbaldehyde in step A.

¹H NMR (400 MHz, CD₃OD) δ 8.74 (s, 2H), 8.37 (d, J=2.8 Hz, 1H), 8.05 (s, 1H), 7.66-7.58 (m, 1H), 7.52-7.43 (m, 1H), 7.36-7.23 (m, 2H), 6.42-6.31 (m, 1H), 6.24 (q, J=6.8 Hz, 1H), 2.87 (d, J=6.8 Hz, 2H), 2.08-2.00 (m, 5H), 1.93-1.83 (m, 2H), 1.28-1.17 (m, 1H), 0.48-0.37 (m, 2H), 0.32-0.20 (m, 2H). ¹⁹F NMR (376 MHz, CD₃OD) δ −78.33, −81.40, −133.56, −141.89. LC-MS: m/z 659.2 (M+H)⁺.

### 4-{4-[(5-chloro-2-pyrimidinyl)methyl]-2,2-dimethyl-3-oxo-3,4-dihydro-2H-1,4-benzoxazin-7-yl}-6-(cyclopropylmethyl)-1-[1-(5-fluoro-2-pyridyl)cyclopropyl]-1,2,7-triaza-1H-indene-5-carboxamide (Compound 211)

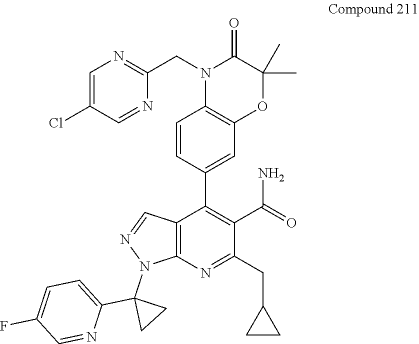 [SMILES: CC1(C)Oc2cc(-c3c(C(N)=O)c(CC4CC4)nc4c3cnn4C3(c4ccc(F)cn4)CC3)ccc2N(Cc2ncc(Cl)cn2)C1=O]

Compound 211 was synthesized following the similar route of Example 56, using 4-((5-chloropyrimidin-2-yl)methyl)-2,2-dimethyl-3-oxo-3,4-dihydro-2H-benzo[b][1,4]oxazine-7-carbaldehyde in step G.

¹H NMR (400 MHz, DMSO-d₆) δ 8.94 (s, 2H), 8.51 (d, J=2.8 Hz, 1H), 8.07 (s, 1H), 7.83 (s, 1H), 7.57 (s, 1H), 7.48-7.53 (m, 1H), 7.34 (d, J=2.0 Hz, 1H), 7.28 (dd, J₁=2.0 Hz, J₂=8.4 Hz, 1H), 7.12 (d, J=8.4 Hz, 1H), 6.30-6.33 (m, 1H), 5.35 (s, 2H), 2.73 (d, J=7.2 Hz, 2H), 1.92-1.96 (m, 2H), 1.80-1.83 (m, 2H), 1.52 (s, 6H), 1.13-1.20 (m, 1H), 0.34-0.38 (m, 2H), 0.20-0.24 (m, 2H). ¹⁹F NMR (376 MHz, DMSO-d₆) δ −131.56. LC-MS: m/z 653.5 (M+H)⁺.

### 6-(cyclopropylmethyl)-1-[1-(5-fluoro-2-pyridyl)cyclopropyl]-4-{4-[(5-fluoro-2-pyrimidinyl)methyl]-2,2-dimethyl-3-oxo-3,4-dihydro-2H-1,4-benzoxazin-7-yl}-1,2,7-triaza-1H-indene-5-carboxamide (Compound 212)

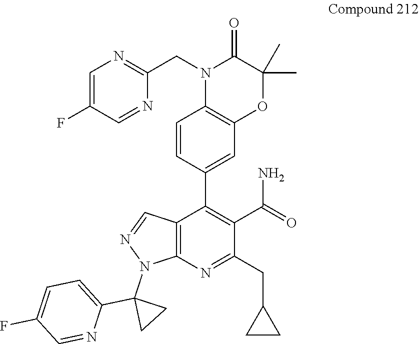 [SMILES: CC1(C)Oc2cc(-c3c(C(N)=O)c(CC4CC4)nc4c3cnn4C3(c4ccc(F)cn4)CC3)ccc2N(Cc2ncc(F)cn2)C1=O]

Compound 212 was synthesized following the similar route of Example 56, using 4-((5-fluoropyrimidin-2-yl)methyl)-2,2-dimethyl-3-oxo-3,4-dihydro-2H-benzo[b][1,4]oxazine-7-carbaldehyde in step G.

¹H NMR (400 MHz, DMSO-d₆) δ 8.90 (s, 2H), 8.51 (d, J=2.8 Hz, 1H), 8.06 (s, 1H), 7.83 (d, J=1.6 Hz, 1H), 7.57 (d, J=1.6 Hz, 1H), 7.48-7.53 (m, 1H), 7.34 (d, J=2.0 Hz, 1H), 7.27 (dd, J₁=1.6 Hz, J₂=8 Hz, 1H), 7.10 (d, J=8 Hz, 1H), 6.30-6.33 (m, 1H), 5.36 (s, 2H), 2.73 (d, J=7.2 Hz, 2H), 1.92-1.96 (m, 2H), 1.80-1.83 (m, 2H), 1.52 (s, 6H), 1.13-1.20 (m, 1H), 0.34-0.37 (m, 2H), 0.21-0.24 (m, 2H). ¹⁹F NMR (376 MHz, DMSO-d₆) δ −131.55, −140.00. LC-MS: m/z 637.5 (M+H)⁺.

### 4-{4-[(5-chloro-2-pyridyl)methyl]-2,2-dimethyl-3-oxo-3,4-dihydro-2H-1,4-benzoxazin-7-yl}-6-(cyclopropylmethyl)-1-[1-(5-fluoro-2-pyridyl)cyclopropyl]-1,2,7-triaza-1H-indene-5-carboxamide (Compound 213)

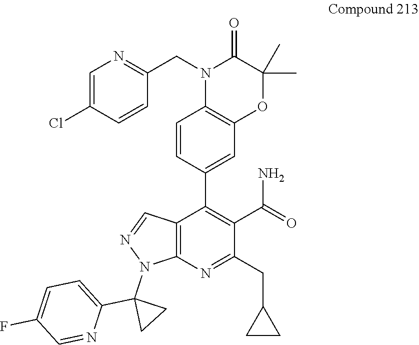 [SMILES: CC1(C)Oc2cc(-c3c(C(N)=O)c(CC4CC4)nc4c3cnn4C3(c4ccc(F)cn4)CC3)ccc2N(Cc2ccc(Cl)cn2)C1=O]

Compound 213 was synthesized following the similar route of Example 56, using 4-[(5-chloropyridin-2-yl)methyl]-2,2-dimethyl-3-oxo-3,4-dihydro-2H-benzo[b][1,4]oxazine-7-carbaldehyde in step G.

¹H NMR (400 MHz, DMSO-d₆) δ 8.59 (d, J=2.4 Hz, 1H), 8.51 (d, J=3.2 Hz, 1H), 8.06 (s, 1H), 7.95 (dd, J₁=2.4 Hz, J₂=10.8 Hz, 1H), 7.82 (d, J=1.6 Hz, 1H), 7.56 (d, J=2.0 Hz, 1H), 7.48-7.53 (m, 1H), 7.41 (d, J=8.4 Hz, 1H), 7.33 (d, J=1.6 Hz, 1H), 7.29 (d, J₁=2.0 Hz, J₂=8.4 Hz, 1H), 7.16 (d, J=8.8 Hz, 1H), 6.31 (dd, J₁=4.0 Hz, J₂=8.8 Hz, 1H), 5.267 (s, 2H), 2.73 (d, J=7.2 Hz, 2H), 1.92-1.95 (m, 2H), 1.80-1.83 (m, 2H), 1.53 (s, 6H), 1.12-1.20 (m, 1H), 0.33-0.38 (m, 2H), 0.20-0.23 (m, 2H). ¹⁹F NMR (376 MHz, DMSO-d₆) δ −131.56. LC-MS: m/z 652.4 (M+H)⁺.

### 6-(cyclopropylmethyl)-1-[1-(5-fluoro-2-pyridyl)cyclopropyl]-4-{4-[(5-fluoro-2-pyridyl)methyl]-2,2-dimethyl-3-oxo-3,4-dihydro-2H-1,4-benzoxazin-7-yl}-1,2,7-triaza-1H-indene-5-carboxamide (Compound 214)

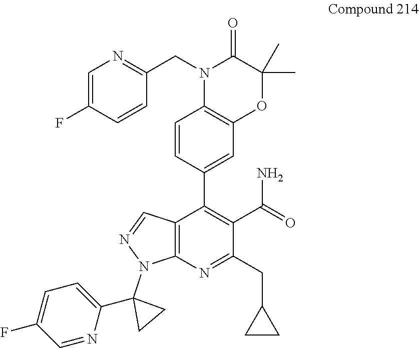 [SMILES: CC1(C)Oc2cc(-c3c(C(N)=O)c(CC4CC4)nc4c3cnn4C3(c4ccc(F)cn4)CC3)ccc2N(Cc2ccc(F)cn2)C1=O]

Compound 214 was synthesized following the similar route of Example 56, using 4-((5-fluoropyridin-2-yl)methyl)-2,2-dimethyl-3-oxo-3,4-dihydro-2H-benzo[b][1,4]oxazine-7-carbaldehyde in step G.

¹H NMR (400 MHz, DMSO-d₆) δ 8.54 (d, J=2.8 Hz, 1H), 8.51 (d, J=2.4 Hz, 1H), 8.06 (s, 1H), 7.82 (s, 1H), 7.73-7.78 (s, 1H), 7.56 (s, 1H), 7.48-7.53 (m, 1H), 7.44 (dd, J₁=4.4 Hz, J₂=8.4 Hz, 1H), 7.32 (s, 1H), 7.29 (d, J=8.4 Hz, 1H), 7.18 (d, J=8 Hz, 1H), 6.29-6.33 (m, 1H), 5.26 (s, 2H), 2.73 (d, J=7.2 Hz, 2H), 1.92-1.94 (m, 2H), 1.81-1.83 (m, 2H), 1.53 (s, 6H), 1.13-1.19 (m, 1H), 0.34-0.36 (m, 2H), 0.21-0.22 (m, 2H). ¹⁹F NMR (376 MHz, DMSO-d₆) δ −129.41, −131.55. LC-MS: m/z 637.0 (M+H)⁺.

### 4-{4-[(5-chloro-2-pyrimidinyl)methyl]-3-oxo-3,4-dihydrospiro[1,4-benzoxazine-2,1′-cyclobutan]-7-yl}-6-(cyclopropylmethyl)-1-[1-(5-fluoro-2-pyridyl)cyclopropyl]-1,2,7-triaza-1H-indene-5-carboxamide (Compound 215)

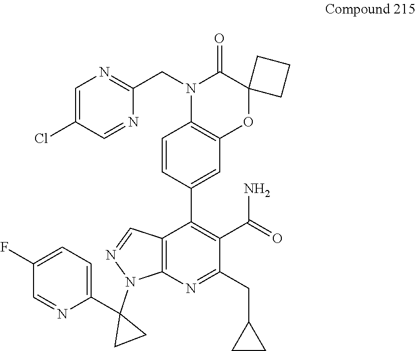 [SMILES: NC(=O)c1c(CC2CC2)nc2c(cnn2C2(c3ccc(F)cn3)CC2)c1-c1ccc2c(c1)OC1(CCC1)C(=O)N2Cc1ncc(Cl)cn1]

Compound 215 was synthesized following the similar route of Example 56, using 4-((5-chloropyrimidin-2-yl)methyl)-3-oxo-3,4-dihydrospiro[benzo[b][1,4]oxazine-2,1′-cyclobutane]-7-carbaldehyde in step G.

¹H NMR (400 MHz, CD₃OD) δ 8.79 (s, 2H), 8.37 (d, J=2.4 Hz, 1H), 8.04 (s, 1H), 7.40-7.44 (m, 1H), 7.25-7.34 (m, 2H), 7.01 (d, J=8.4 Hz, 1H), 6.34 (dd, J=8.8, 4.0 Hz, 1H), 5.40 (s, 2H), 2.86 (d, J=7.2 Hz, 2H), 2.68-2.76 (m, 2H), 2.38-2.48 (m, 2H), 1.87-2.04 (m, 6H), 1.20-1.27 (m, 1H), 0.38-0.46 (m, 2H), 0.22-0.31 (m, 2H). ¹⁹F NMR (376 MHz, CD₃OD) δ −113.63. LC-MS: m/z 665.4 (M+H)⁺.

### 4-{4-[(5-chloro-2-pyrimidinyl)methyl]-3-oxo-3,4-dihydrospiro[1,4-benzoxazine-2,3′-oxetan]-7-yl}-6-(cyclopropylmethyl)-1-[1-(5-fluoro-2-pyridyl)cyclopropyl]-1,2,7-triaza-1H-indene-5-carboxamide (Compound 302)

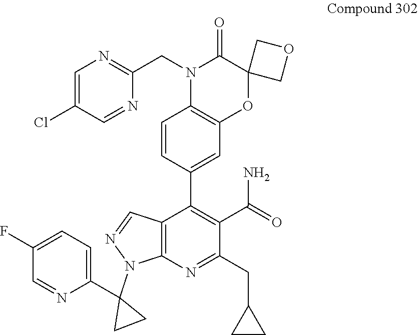 [SMILES: NC(=O)c1c(CC2CC2)nc2c(cnn2C2(c3ccc(F)cn3)CC2)c1-c1ccc2c(c1)OC1(COC1)C(=O)N2Cc1ncc(Cl)cn1]

¹H NMR (400 MHz, CDCl₃) δ 8.65 (s, 2H), 8.36 (d, J=3.2 Hz, 1H), 7.78 (s, 1H), 7.38-7.34 (m, 1H), 7.33-7.30 (m, 1H), 7.14-7.08 (m, 2H), 6.30 (dd, J=4.4, 8.8 Hz, 1H), 5.52 (br s, 1H), 5.33-5.37 (m, 3H), 5.22 (d, J=7.2 Hz, 2H), 4.90 (d, J=7.2 Hz, 2H), 2.86 (d, J=7.2 Hz, 2H), 2.08-2.03 (m, 2H), 1.90-1.84 (m, 2H), 1.24-1.18 (m, 1H), 0.47-0.39 (m, 2H), 0.26 (q, J=5.2 Hz, 2H). ¹⁹F NMR (376 MHz, CDCl₃) δ −131.52. LC-MS: m/z 667.3 (M+H)⁺.

### 4-{4-[(5-chloro-2-pyrimidinyl)methyl]-2-fluoro-3-oxo-3,4-dihydro-2H-1,4-benzoxazin-7-yl}-6-(cyclopropylmethyl)-1-[1-(5-fluoro-2-pyridyl)cyclopropyl]-1,2,7-triaza-1H-indene-5-carboxamide (Compound 303)

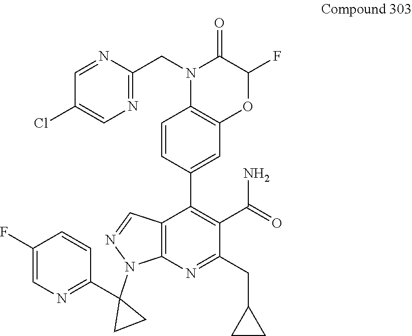 [SMILES: NC(=O)c1c(CC2CC2)nc2c(cnn2C2(c3ccc(F)cn3)CC2)c1-c1ccc2c(c1)OC(F)C(=O)N2Cc1ncc(Cl)cn1]

Compound 303 was synthesized following the similar route of Example 56, using 4-((5-chloropyrimidin-2-yl)methyl)-2-fluoro-3-oxo-3,4-dihydro-2H-benzo[b][1,4]oxazine-7-carbaldehyde in step A.

¹H NMR (400 MHz, DMSO-d₆) δ 8.97 (s, 2H), 8.51 (br s, 1H), 8.01-8.16 (m, 1H), 7.90 (s, 1H), 7.34-7.67 (m, 5H), 6.50-6.80 (m, 1H), 6.34 (dd, J=8.4, 4.0 Hz, 1H), 5.27-5.65 (m, 2H), 2.74 (d, J=6.4 Hz, 2H), 1.94 (s, 2H), 1.83 (s, 2H), 1.17 (s, 1H), 0.37 (d, J=6.4 Hz, 2H), 0.22 (d, J=3.2 Hz, 2H). ¹⁹F NMR (376 MHz, DMSO-d₆) δ −127.29, −131.51. LC-MS: m/z 643.3 (M+H)⁺.

### 6-(cyclopropylmethyl)-1-[1-(5-fluoro-2-pyridyl)cyclopropyl]-4-{3-[(5-fluoro-2-pyrimidinyl)methyl]-2-oxo-2,3-dihydro-1,3-benzoxazol-6-yl}-1,2,7-triaza-1H-indene-5-carboxamide (Compound 216)

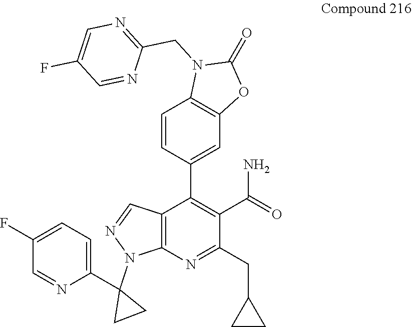 [SMILES: NC(=O)c1c(CC2CC2)nc2c(cnn2C2(c3ccc(F)cn3)CC2)c1-c1ccc2c(c1)oc(=O)n2Cc1ncc(F)cn1]

Compound 216 was synthesized following the similar route of Example 56, using 3-((5-fluoropyrimidin-2-yl)methyl)-2-oxo-2,3-dihydrobenzo[d]oxazole-6-carbaldehyde in step G.

¹H NMR (400 MHz, DMSO-d₆) δ 8.93 (s, 2H), 8.51 (d, J=2.8 Hz, 1H), 8.11 (s, 1H), 7.86 (d, J=1.6 Hz, 1H), 7.68 (d, J=1.2 Hz, 1H), 7.57 (d, J=1.6 Hz, 1H), 7.49-7.55 (m, 2H), 7.40 (d, J=8.4 Hz, 1H), 6.35 (dd, J₁=4.4 Hz, J₂=9.2 Hz, 1H), 5.39 (s, 2H), 2.75 (d, J=6.8 Hz, 2H), 1.93-1.97 (m, 2H), 1.82-1.84 (m, 2H), 1.15-1.23 (m, 1H), 0.35-0.39 (m, 2H), 0.21-0.25 (m, 2H). ¹⁹F NMR (376 MHz, DMSO-d₆) δ −131.53, −139.28. LC-MS: m/z 595.3 (M+H)⁺.

### 6-(cyclopropylmethyl)-4-{2,2-difluoro-4-[(5-fluoro-2-pyrimidinyl)methyl]-3-oxo-3,4-dihydro-2H-1,4-benzoxazin-7-yl}-1-[(S)-1-(p-fluorophenyl)ethyl]-1,2,7-triaza-1H-indene-5-carboxamide (Compound 217)

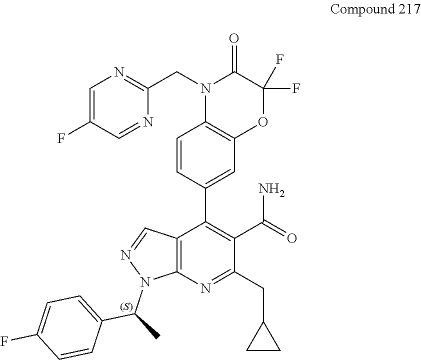

Compound 217 was synthesized following the similar route of Example 55, using ethyl 1H-pyrazole-5-carboxylate and (R)-1-(4-fluorophenyl) ethan-1-ol in step B and then following the similar route of Example 56, using 4-((5-fluoropyrimidin-2-yl)methyl)-2,2-difluoro-3-oxo-3,4-dihydro-2H-benzo[b][1,4]oxazine-7-carbaldehyde in step G.

¹H NMR (400 MHz, DMSO-d₆) δ 8.93 (s, 2H), 7.96 (s, 1H), 7.84 (d, J=1.6 Hz, 1H), 7.61 (d, J=1.6 Hz, 1H), 7.60 (d, J=1.6 Hz, 1H), 7.41-7.48 (m, 4H), 7.07 (t, J=8.8 Hz, 2H), 6.29 (dd, J₁=7.2 Hz, J₂=14 Hz, 1H), 5.51 (s, 2H), 2.74-2.87 (m, 2H), 1.96 (d, J=7.2 Hz, 3H), 1.23-1.32 (m, 1H), 0.44-0.47 (m, 2H), 0.28-0.31 (m, 2H). ¹⁹F NMR (376 MHz, DMSO-d₆) δ −74.90, −115.02, −139.27. LC-MS: m/z 632.5 (M+H)⁺.

### 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(S)-1-(5-fluoro-2-pyridyl)ethyl]-1,2,7-triaza-1H-indene-5-carboxamide and 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(R)-1-(5-fluoro-2-pyridyl)ethyl]-1,2,7-triaza-1H-indene-5-carboxamide (Compound 218 and Compound 219)

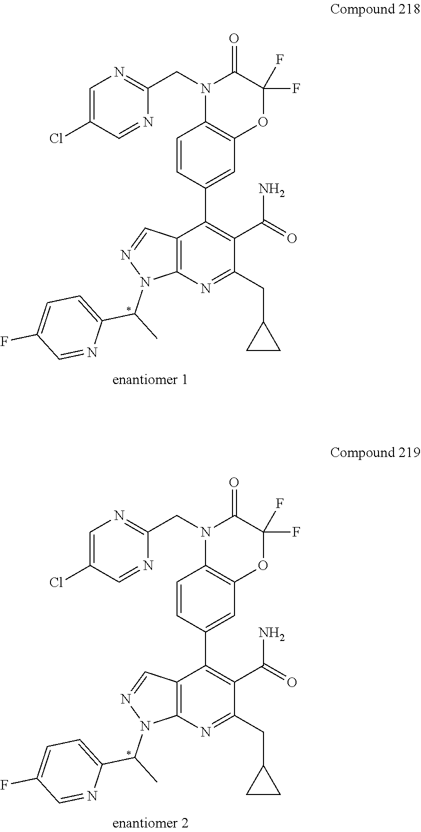 [SMILES: CC(c1ccc(F)cn1)n1ncc2c(-c3ccc4c(c3)OC(F)(F)C(=O)N4Cc3ncc(Cl)cn3)c(C(N)=O)c(CC3CC3)nc21 ; CC(c1ccc(F)cn1)n1ncc2c(-c3ccc4c(c3)OC(F)(F)C(=O)N4Cc3ncc(Cl)cn3)c(C(N)=O)c(CC3CC3)nc21]

4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(1-(5-fluoro-2-pyridyl)ethyl]-1,2,7-triaza-1H-indene-5-carboxamide which was synthesized following the similar route of Example 56, using 2-(5-fluoropyridin-2-yl) propanoic acid in step A (100.0 mg) was purified by SFC (system: Waters SFC 80Q; Column name: DAICEL CHIRALCEL OD; Column size: (250 mm*30 mm, 10 μm); mobile phase A: Supercritical CO₂; mobile phase B: EtOH (+0.1% NH₃·H₂O); Gradient: A/B=60/40; Flow rate: 80 mL/min; Column Temp: RT) to afford 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(S)-1-(5-fluoro-2-pyridyl)ethyl]-1,2,7-triaza-1H-indene-5-carboxamide (26.17 mg) and 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(R)-1-(5-fluoro-2-pyridyl)ethyl]-1,2,7-triaza-1H-indene-5-carboxamide (10.39 mg).

Compound 218, enantiomer 1: Rt=3.20 min. ¹H NMR (400 MHz, DMSO-d₆) δ 8.98 (s, 2H), 8.51 (d, J=2.8 Hz, 1H), 7.99 (s, 1H), 7.85 (s, 1H), 7.68-7.61 (m, 2H), 7.60 (s, 1H), 7.51-7.42 (m, 2H), 7.31-7.27 (m, 1H), 6.39-6.29 (m, 1H), 5.52 (s, 2H), 2.85-2.75 (m, 2H), 1.98 (d, J=7.2 Hz, 3H), 1.30-1.20 (m, 1H), 0.45-0.37 (m, 2H), 0.30-0.20 (m, 2H). ¹⁹F NMR (376 MHz, DMSO-d₆) ¿-74.87, −129.34. LC-MS: m/z 649.2 (M+H)⁺.

Compound 219, enantiomer 2: Rt=3.36 min. ¹H NMR (400 MHz, DMSO-d₆) δ 8.98 (s, 2H), 8.51 (d, J=2.8 Hz, 1H), 7.99 (s, 1H), 7.85 (s, 1H), 7.68-7.61 (m, 2H), 7.60 (s, 1H), 7.51-7.42 (m, 2 H), 7.31-7.27 (m, 1H), 6.39-6.29 (m, 1H), 5.51 (s, 2H), 2.85-2.75 (m, 2H), 1.98 (d, J=6.8 Hz, 3H), 1.30-1.20 (m, 1H), 0.45-0.37 (m, 2H), 0.30-0.20 (m, 2H). ¹⁹F NMR (376 MHz, DMSO-d₆) δ −74.88, −129.34. LC-MS: m/z 649.1 (M+H)⁺.

### 6-(cyclopropylmethyl)-4-{2,2-difluoro-4-[(5-fluoro-2-pyridyl)methyl]-3-oxo-3,4-dihydro-2H-1,4-benzoxazin-7-yl}-1-[1-(5-fluoro-2-pyridyl)cyclopropyl]-1,2,7-triaza-1H-indene-5-carboxamide (Compound 220)

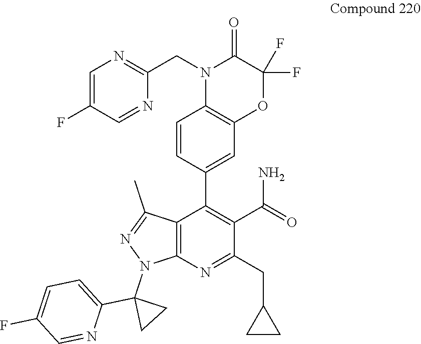 [SMILES: Cc1nn(C2(c3ccc(F)cn3)CC2)c2nc(CC3CC3)c(C(N)=O)c(-c3ccc4c(c3)OC(F)(F)C(=O)N4Cc3ncc(F)cn3)c12]

Compound 220 was synthesized following the similar route of Example 56, using ethyl 2-(methoxyimino)-4-oxopentanoate in step D.

¹H NMR (400 MHz, DMSO-d₆) δ 8.94 (s, 2H), 8.51 (d, J=2.8 Hz, 1H), 7.83 (s, 1H), 7.49-7.55 (m, 3H), 7.40 (d, J=8.8 Hz, 1H), 7.32 (dd, J₁=1.6 Hz, J₂=8.4 Hz, 1H), 6.36 (dd, J₁=4.4 Hz, J₂=8.8 Hz, 1H), 5.52-5.54 (m, 2H), 2.70-2.73 (m, 2H), 1.92 (s, 3H), 1.89-1.91 (m, 2H), 1.76-1.79 (m, 2H), 1.14-1.18 (m, 1H), 0.35-0.39 (m, 2H), 0.20-0.24 (m, 2H). ¹⁹F NMR (376 MHz, DMSO-d₆) δ −73.86, −131.63, −139.30. LC-MS: m/z 659.9 (M+H)⁺.

### Example 57

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(4,4-difluorocyclohexyl)methyl]-1,2,7-triaza-1H-indene-5-carboxamide (Compound 221)

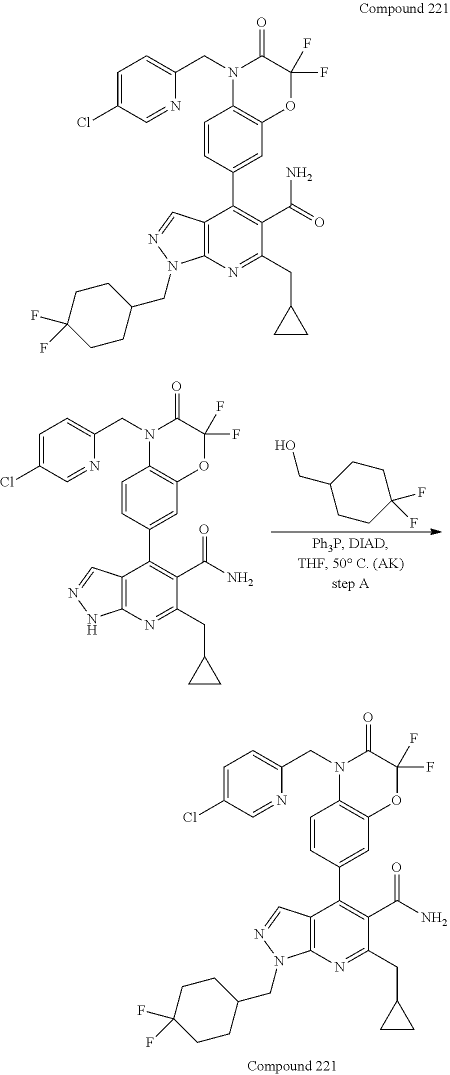 [SMILES: NC(=O)c1c(CC2CC2)nc2c(cnn2CC2CCC(F)(F)CC2)c1-c1ccc2c(c1)OC(F)(F)C(=O)N2Cc1ccc(Cl)cn1 ; NC(=O)c1c(CC2CC2)nc2[nH]ncc2c1-c1ccc2c(c1)OC(F)(F)C(=O)N2Cc1ccc(Cl)cn1 ; OCC1CCC(F)(F)CC1 ; NC(=O)c1c(CC2CC2)nc2c(cnn2CC2CCC(F)(F)CC2)c1-c1ccc2c(c1)OC(F)(F)C(=O)N2Cc1ccc(Cl)cn1]

To a solution of 4-(4-((5-chloropyridin-2-yl)methyl)-2,2-difluoro-3-oxo-3,4-dihydro-2H-benzo[b][1,4]oxazin-7-yl)-6-(cyclopropylmethyl)-1H-pyrazolo[3,4-b]pyridine-5-carboxamide which was synthesized following the similar route of Example 51, using 1H-pyrazol-5-amine, 4-[(5-chloropyridin-2-yl)methyl]-2,2-dimethyl-3-oxo-3,4-dihydro-2H-benzo[b][1,4]oxazine-7-carbaldehyde and tert-butyl(4-cyclopropyl-3-oxobutanoyl)carbamate in step G (50.0 mg, 95.26 μmol), (4,4-difluorocyclohexyl)methanol (57.22 mg, 381.02 μmol) and Ph₃P (74.95 mg, 285.77 μmol) in THF (2.0 mL) was added DIAD (57.78 mg, 285.77 μmol, 55.40 μL) at 0° C. and the mixture was stirred at 50° C. for 2 hours under N₂. The volatiles were removed in vacuo to give a crude. The crude was purified by prep-HPLC (column: C18 150×30 mm; mobile phase: [water (FA)-ACN]; gradient: 60%-90% B over 7 min) to 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(4,4-difluorocyclohexyl)methyl]-1,2,7-triaza-1H-indene-5-carboxamide (37.86 mg, 60.20%). ¹H NMR (400 MHz, DMSO-d₆) δ 8.59 (d, J=2.4 Hz, 1H), 8.00 (dd, J=8.4, 2.4 Hz, 1H), 7.94 (s, 1H), 7.91-7.86 (m, 1H), 7.65-7.57 (m, 3H), 7.52-7.41 (m, 2H), 5.43 (s, 2H), 4.42 (d, J=7.2 Hz, 2H), 2.81 (d, J=6.8 Hz, 2H), 2.22-2.08 (m, 1H), 2.07-1.93 (m, 2H), 1.88-1.68 (m, 2H), 1.67-1.55 (m, 2H), 1.37-1.24 (m, 3H), 0.53-0.41 (m, 2H), 0.38-0.25 (m, 2H). ¹⁹F NMR (376 MHz, DMSO-d₆) δ −90.04, −98.67. LC-MS: m/z 657.2 (M+H)⁺.

### methyl 4-[(5-carbamoyl-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-inden-1-yl)methyl]-1-piperidinecarboxylate (Compound 222)

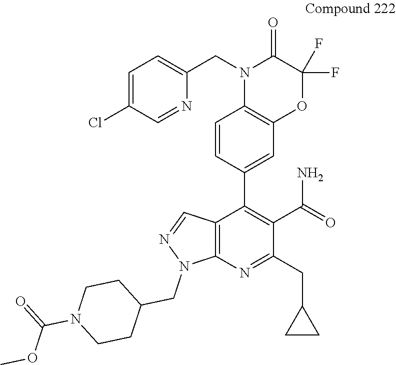 [SMILES: COC(=O)N1CCC(Cn2ncc3c(-c4ccc5c(c4)OC(F)(F)C(=O)N5Cc4ccc(Cl)cn4)c(C(N)=O)c(CC4CC4)nc32)CC1]

Compound 222 was synthesized following the similar route of Example 57, using methyl 4-(hydroxymethyl) piperidine-1-carboxylate in step A.

¹H NMR (400 MHz, DMSO-d₆) δ 8.59 (d, J=2.8 Hz, 1H), 8.00 (dd, J=8.8, 2.8 Hz, 1H), 7.93 (s, 1H), 7.88 (s, 1H), 7.64-7.57 (m, 3H), 7.52-7.41 (m, 2H), 5.42 (s, 2H), 4.39 (d, J=6.8 Hz, 2H), 3.99-3.85 (m, 2H), 3.55 (s, 3H), 2.84-2.64 (m, 4H), 2.25-2.12 (m, 1H), 1.55-1.45 (m, 2H), 1.34-1.24 (m, 1H), 1.20-1.12 (m, 2H), 0.50-0.42 (m, 2H), 0.34-0.25 (m, 2H). ¹⁹F NMR (376 MHz, DMSO-d₆) δ −74.47. LC-MS: m/z 680.2.1 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(tetrahydro-4H-pyran-4-yl)methyl]-1,2,7-triaza-1H-indene-5-carboxamide (Compound 223)

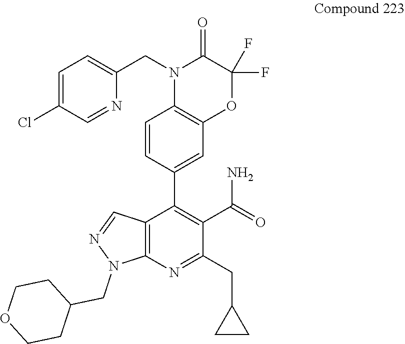 [SMILES: NC(=O)c1c(CC2CC2)nc2c(cnn2CC2CCOCC2)c1-c1ccc2c(c1)OC(F)(F)C(=O)N2Cc1ccc(Cl)cn1]

Compound 223 was synthesized following the similar route of Example 57, using (tetrahydro-2H-pyran-4-yl) methanol in step A.

¹H NMR (400 MHz, CDCl₃) δ 8.57 (d, J=2.8 Hz, 1H), 7.85 (s, 1H), 7.71 (dd, J=8.4, 2.4 Hz, 1H), 7.52 (d, J=1.2 Hz, 1H), 7.49-7.42 (m, 2H), 7.36 (d, J=8.4 Hz, 1H), 5.68-5.56 (m, 1H), 5.48-5.39 (m, 1H), 5.35 (s, 2H), 4.46 (d, J=7.2 Hz, 2H), 4.00-3.92 (m, 2H), 3.42-3.33 (m, 2H), 2.95 (d, J=6.8 Hz, 2H), 2.43-2.29 (m, 1H), 1.52-1.46 (m, 4H), 1.37-1.31 (m, 1H), 0.57-0.51 (m, 2H), 0.38-0.32 (m, 2H). ¹⁹F NMR (400 MHz, CDCl₃) δ −76.94. LC-MS: m/z 623.3 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(2,2-dimethyltetrahydro-4H-pyran-4-yl)methyl]-1,2,7-triaza-1H-indene-5-carboxamide (Compound 224)

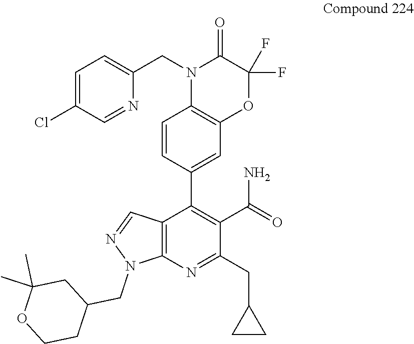 [SMILES: CC1(C)CC(Cn2ncc3c(-c4ccc5c(c4)OC(F)(F)C(=O)N5Cc4ccc(Cl)cn4)c(C(N)=O)c(CC4CC4)nc32)CCO1]

Compound 224 was synthesized following the similar route of Example 57, using (2,2-dimethyltetrahydro-2H-pyran-4-yl) methanol in step A.

¹H NMR (400 MHz, DMSO-d₆) δ 8.59 (d, J=2.4 Hz, 1H), 8.01 (dd, J=8.4, 2.4 Hz, 1H), 7.94 (s, 1H), 7.91-7.84 (m, 1H), 7.65-7.56 (m, 3H), 7.53-7.40 (m, 2H), 5.43 (s, 2H), 4.33 (d, J=6.4 Hz, 2H), 3.61-3.43 (m, 2H), 2.89-2.73 (m, 2H), 2.47-2.35 (m, 1H), 1.46-1.38 (m, 1H), 1.37-1.25 (m, 2H), 1.21-1.11 (m, 2H), 1.09 (s, 6H), 0.52-0.40 (m, 2H), 0.35-0.27 (m, 2H). ¹⁹F NMR (376 MHz, DMSO-d₆) δ −74.48. LC-MS: m/z 651.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(perhydro-3-furyl)methyl]-1,2,7-triaza-1H-indene-5-carboxamide (Compound 225)

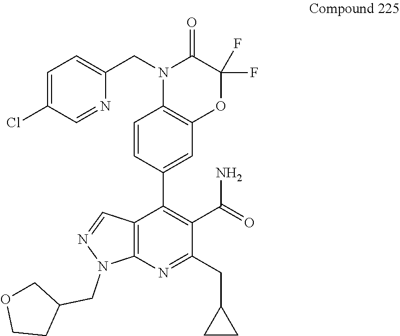 [SMILES: NC(=O)c1c(CC2CC2)nc2c(cnn2CC2CCOC2)c1-c1ccc2c(c1)OC(F)(F)C(=O)N2Cc1ccc(Cl)cn1]

Compound 225 was synthesized following the similar route of Example 57, using (tetrahydrofuran-3-yl) methanol in step A.

¹H NMR (400 MHz, DMSO-d₆) δ 8.60 (d, J=2.4 Hz, 1H), 8.01 (dd, J=8.4, 2.4 Hz, 1H), 7.95 (s, 1H), 7.89 (s, 1H), 7.65-7.58 (m, 3H), 7.52-7.42 (m, 2H), 5.44 (s, 2H), 4.56-4.40 (m, 2H), 3.86-3.73 (m, 1H), 3.69-3.55 (m, 3H), 2.90-2.78 (m, 3H), 1.97-1.85 (m, 1H), 1.73-1.63 (m, 1H), 1.35-1.24 (m, 1H), 0.51-0.43 (m, 2H), 0.35-0.28 (m, 2H). ¹⁹F NMR (376 MHz, DMSO-d₆) δ −74.46. LC-MS: m/z 609.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(1,4-dioxa-8-spiro[4.5]decyl)methyl]-1,2,7-triaza-1H-indene-5-carboxamide (Compound 226)

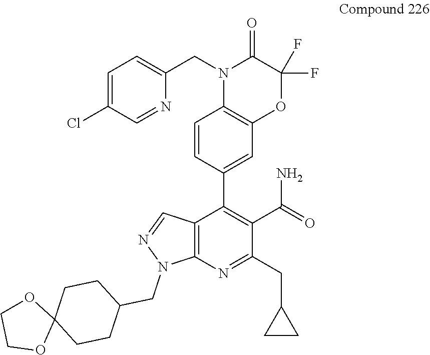 [SMILES: NC(=O)c1c(CC2CC2)nc2c(cnn2CC2CCC3(CC2)OCCO3)c1-c1ccc2c(c1)OC(F)(F)C(=O)N2Cc1ccc(Cl)cn1]

Compound 226 was synthesized following the similar route of Example 57, using (1,4-dioxaspiro[4.5]decan-8-yl) methanol in step A. ¹H NMR (400 MHz, CDCl₃) δ 8.57 (d, J=2.4 Hz, 1H), 7.84 (s, 1H), 7.71 (dd, J=8.0, 2.0 Hz, 1H), 7.53 (d, J=2.0 Hz, 1H), 7.50-7.42 (m, 2H), 7.36 (d, J=8.8 Hz, 1H), 5.61-5.57 (m, 1H), 5.46-5.40 (m, 1H), 5.35 (s, 2H), 4.45 (d, J=6.8 Hz, 2H), 3.94 (s, 4H), 2.94 (d, J=7.2 Hz, 2H), 2.21-2.08 (m, 1H), 1.82-1.73 (m, 2H), 1.66-1.60 (m, 2H), 1.53-1.48 (m, 2H), 1.47-1.40 (m, 2H), 1.36-1.30 (m, 1H), 0.59-0.48 (m, 2H), 0.39-0.32 (m, 2H). ¹⁹F NMR (400 MHz, CDCl₃) δ −76.95. LC-MS: m/z 679.3 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-(tetrahydro-4H-pyran-4-yl)ethyl]-1,2,7-triaza-1H-indene-5-carboxamide (Compound 227)

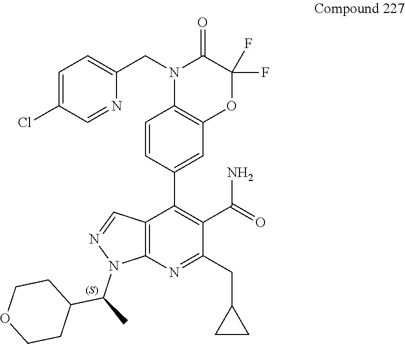

Compound 227 was synthesized following the similar route of Example 57, using (R)-1-(tetrahydro-2H-pyran-4-yl) ethan-1-ol in step A. ¹H NMR (400 MHz, CD₃OD) δ 8.52 (d, J=2.4 Hz, 1H), 7.91 (s, 1H), 7.87 (dd, J=8.4, 2.4 Hz, 1H), 7.57 (d, J=1.6 Hz, 1H), 7.48 (d, J=8.8 Hz, 2H), 7.41 (d, J=8.4 Hz, 1H), 5.44 (s, 2H), 4.97-4.91 (m, 1H), 4.05-3.92 (m, 1H), 3.83-3.69 (m, 1H), 3.46-3.36 (m, 1H), 3.27-3.16 (m, 1H), 3.03-2.80 (m, 2H), 2.32-2.20 (m, 1H), 1.94-1.82 (m, 1H), 1.60 (d, J=7.2 Hz, 3H), 1.44-1.32 (m, 2H), 1.26-1.16 (m, 1H), 0.90-0.78 (m, 1H), 0.57-0.47 (m, 2H), 0.40-0.30 (m, 2H). ¹⁹F NMR (376 MHz, CD₃OD) δ −78.20. LC-MS: m/z 637.4 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[1-(3-oxetanyl)ethyl]-1,2,7-triaza-1H-indene-5-carboxamide (Compound 228)

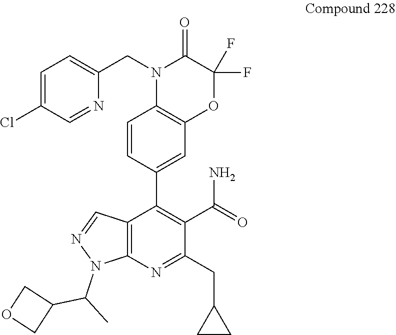 [SMILES: CC(C1COC1)n1ncc2c(-c3ccc4c(c3)OC(F)(F)C(=O)N4Cc3ccc(Cl)cn3)c(C(N)=O)c(CC3CC3)nc21]

Compound 228 was synthesized following the similar route of Example 57, using 1-(oxetan-3-yl)ethan-1-ol in step A. ¹H NMR (400 MHz, DMSO-d₆) δ 8.59 (d, J=2.0 Hz, 1H), 8.00 (dd, J=8.4, 2.4 Hz, 1H), 7.92 (s, 1H), 7.89-7.82 (m, 1H), 7.65-7.54 (m, 3H), 7.51-7.40 (m, 2H), 5.48-5.34 (m, 3H), 4.80-4.70 (m, 1H), 4.57-4.50 (m, 1H), 4.49-4.41 (m, 1H), 4.30-4.22 (m, 1H), 3.67-3.54 (m, 1H), 2.90-2.75 (m, 2H), 1.46 (d, J=6.4 Hz, 3H), 1.36-1.27 (m, 1H), 0.52-0.43 (m, 2H), 0.37-0.26 (m, 2H). ¹⁹F NMR (376 MHz, DMSO-d₆) δ −74.45. LC-MS: m/z 609.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-(m-mesylphenyl)ethyl]-1,2,7-triaza-1H-indene-5-carboxamide (Compound 229)

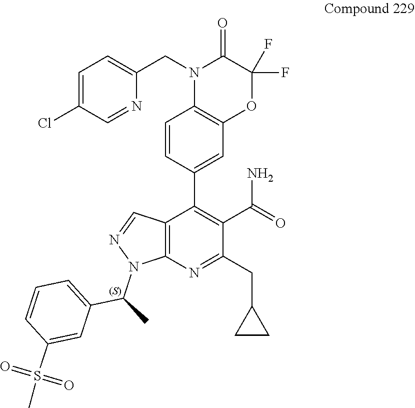

Compound 229 was synthesized following the similar route of Example 57, using (R)-1-(3-(methylsulfonyl)phenyl) ethan-1-ol in step A. ¹H NMR (400 MHz, DMSO-d₆) δ 8.59 (d, J=2.4 Hz, 1H), 8.04-7.98 (m, 3H), 7.87-7.80 (m, 2H), 7.75 (d, J=8.0 Hz, 1H), 7.65-7.57 (m, 4H), 7.53-7.40 (m, 2H), 6.41 (q, J=6.8 Hz, 1H), 5.43 (s, 2H), 3.20 (s, 3H), 2.87-2.74 (m, 2H), 2.01 (d, J=7.2 Hz, 3H), 1.41-1.18 (m, 1H), 0.51-0.40 (m, 2H), 0.34-0.24 (m, 2H). ¹⁹F NMR (376 MHz, DMSO-d₆) δ −74.44. LC-MS: m/z 707.1 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[1-(5-methyl-2-pyridyl)ethyl]-1,2,7-triaza-1H-indene-5-carboxamide (Compound 230)

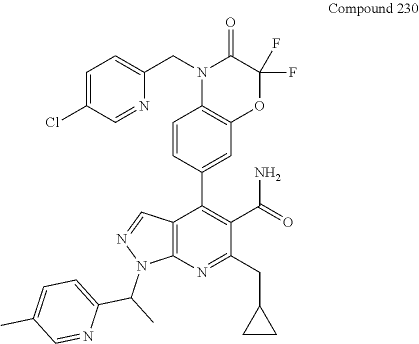 [SMILES: Cc1ccc(C(C)n2ncc3c(-c4ccc5c(c4)OC(F)(F)C(=O)N5Cc4ccc(Cl)cn4)c(C(N)=O)c(CC4CC4)nc32)nc1]

Compound 230 was synthesized following the similar route of Example 57, using 1-(5-methylpyridin-2-yl) ethan-1-ol in step A. ¹H NMR (400 MHz, DMSO-d₆) δ 8.60 (d, J=2.4 Hz, 1H), 8.36-8.32 (m, 1H), 8.00 (dd, J=8.4, 2.4 Hz, 1H), 7.96 (s, 1H), 7.86-7.83 (m, 1H), 7.63-7.57 (m, 3H), 7.54-7.50 (m, 1H), 7.50-7.43 (m, 2H), 7.04 (d, J=8.4 Hz, 1H), 6.28 (q, J=7.2 Hz, 1H), 5.43 (s, 2H), 2.83-2.72 (m, 2H), 2.24 (s, 3H), 1.96 (d, J=6.8 Hz, 3H), 1.31-1.25 (m, 1H), 0.48-0.34 (m, 2H), 0.31-0.17 (m, 2H). ¹⁹F NMR (376 MHz, DMSO-d₆) δ −74.43. LC-MS: m/z 644.2 (M+H)⁺.

### 1-[1-(5-chloro-2-pyridyl)ethyl]-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-indene-5-carboxamide (Compound 231)

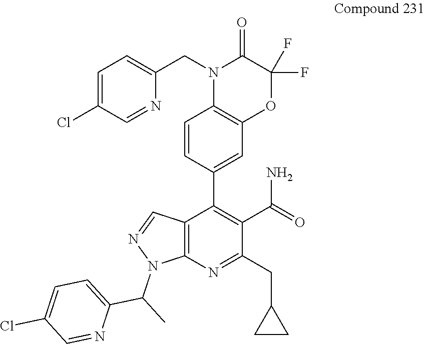 [SMILES: CC(c1ccc(Cl)cn1)n1ncc2c(-c3ccc4c(c3)OC(F)(F)C(=O)N4Cc3ccc(Cl)cn3)c(C(N)=O)c(CC3CC3)nc21]

Compound 231 was synthesized following the similar route of Example 57, using 1-(5-methylpyridin-2-yl)ethan-1-ol in step A. ¹H NMR (400 MHz, CD₃OD) δ 8.52 (d, J=2.0 Hz, 1H), 8.47 (d, J=2.0 Hz, 1H), 7.95 (s, 1H), 7.87 (dd, J=8.4, 2.4 Hz, 1H), 7.73 (dd, J=8.4, 2.4 Hz, 1H), 7.57 (d, J=2.0 Hz, 1H), 7.51-7.45 (m, 2H), 7.41 (d, J=8.8 Hz, 1H), 7.19 (d, J=8.4 Hz, 1H), 6.39 (q, J=7.2 Hz, 1H), 5.44 (s, 2H), 2.91-2.83 (m, 2H), 2.04 (d, J=7.2 Hz, 3H), 1.28-1.22 (m, 1H), 0.50-0.42 (m, 2H), 0.30-0.24 (m, 2H). ¹⁹F NMR (376 MHz, CD₃OD) δ −78.21. LC-MS: m/z 664.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[1-(5-fluoro-2-pyridyl)ethyl]-1,2,7-triaza-1H-indene-5-carboxamide (Compound 232)

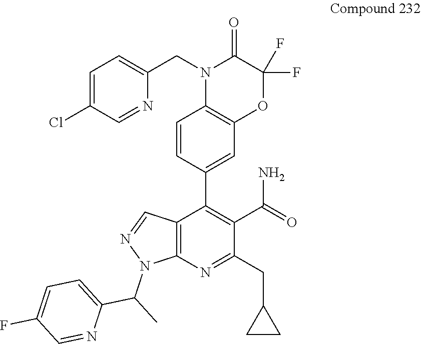 [SMILES: CC(c1ccc(F)cn1)n1ncc2c(-c3ccc4c(c3)OC(F)(F)C(=O)N4Cc3ccc(Cl)cn3)c(C(N)=O)c(CC3CC3)nc21]

Compound 232 was synthesized following the similar route of Example 57, using 1-(5-fluoropyridin-2-yl) ethan-1-ol in step A. ¹H NMR (400 MHz, DMSO-d₆) δ 8.59 (d, J=2.4 Hz, 1H), 8.51 (d, J=2.8 Hz, 1H), 8.00 (dd, J=8.4, 2.4 Hz, 1H), 7.98 (s, 1H), 7.90-7.81 (m, 1H), 7.73-7.64 (m, 1H), 7.63-7.56 (m, 3H), 7.53-7.40 (m, 2H), 7.33-7.22 (m, 1H), 6.34 (q, J=7.2 Hz, 1H), 5.43 (s, 2H), 2.82-2.72 (m, 2H), 1.97 (d, J=7.2 Hz, 3H), 1.26-1.20 (m, 1H), 0.45-0.37 (m, 2H), 0.27-0.20 (m, 2H). ¹⁹F NMR (376 MHz, DMSO-d₆) δ −74.42, −129.31. LC-MS: m/z 648.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-(5-fluoro-2-pyridyl)ethyl]-1,2,7-triaza-1H-indene-5-carboxamide and 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(R)-1-(5-fluoro-2-pyridyl)ethyl]-1,2,7-triaza-1H-indene-5-carboxamide (Compound 233 and Compound 234)

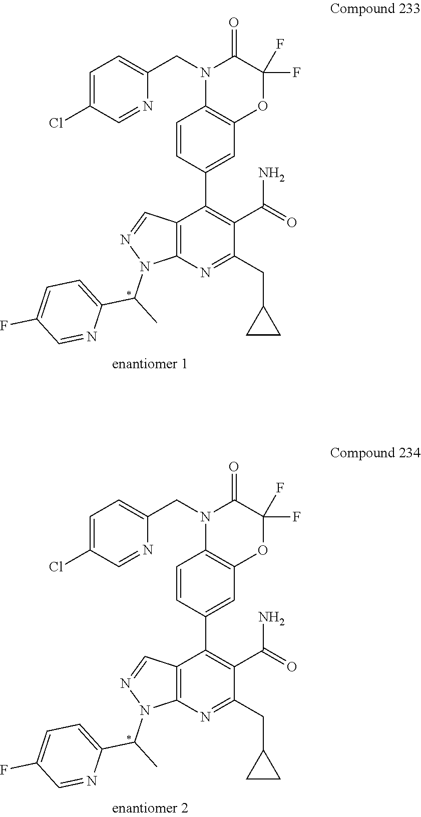 [SMILES: CC(c1ccc(F)cn1)n1ncc2c(-c3ccc4c(c3)OC(F)(F)C(=O)N4Cc3ccc(Cl)cn3)c(C(N)=O)c(CC3CC3)nc21 ; CC(c1ccc(F)cn1)n1ncc2c(-c3ccc4c(c3)OC(F)(F)C(=O)N4Cc3ccc(Cl)cn3)c(C(N)=O)c(CC3CC3)nc21]

4-{4-[(5-chloro-2-pyridyl)methyl]-2,2-difluoro-3-oxo-3,4-dihydro-2H-1,4-benzoxazin-7-yl}-6-(cyclopropylmethyl)-1-[1-(5-fluoro-2-pyridyl)ethyl]-1,2,7-triaza-1H-indene-5-carboxamide (180.0 mg) was purified by SFC (system: Waters SFC 21; Column name: DAICEL CHIRALCEL OD; Column size: (250 mm+30 mm, 10 μm); mobile phase A: Supercritical CO₂; mobile phase B: i-PrOH (+0.1% NH₃·H₂O); Gradient: A/B=60/40; Flow rate: 80 mL/min; Column Temp: RT) to afford 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-(5-fluoro-2-pyridyl)ethyl]-1,2,7-triaza-1H-indene-5-carboxamide (60.04 mg) and 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-(5-fluoro-2-pyridyl)ethyl]-1,2,7-triaza-1H-indene-5-carboxamide (34.59 mg).

Compound 233, enantiomer 1: Rt=4.13 min. ¹H NMR (400 MHz, DMSO-d₆) δ 8.60 (d, J=2.4 Hz, 1H), 8.51 (d, J=2.4 Hz, 1H), 8.05-7.96 (m, 2H), 7.86 (s, 1H), 7.73-7.66 (m, 1H), 7.65-7.55 (m, 3H), 7.55-7.40 (m, 2H), 7.34-7.24 (m, 1H), 6.34 (q, J=7.2 Hz, 1H), 5.44 (s, 2H), 2.86-2.71 (m, 2H), 1.98 (d, J=6.8 Hz, 3H), 1.27-1.19 (m, 1H), 0.48-0.35 (m, 2H), 0.33-0.19 (m, 2H). ¹⁹F NMR (376 MHz, DMSO-d₆) δ −74.46, −129.30. LC-MS: m/z 648.5 (M+H)⁺.

Compound 234, enantiomer 2: Rt=4.33 min. ¹H NMR (400 MHz, DMSO-d₆) δ 8.59 (d, J=2.4 Hz, 1H), 8.51 (d, J=3.2 Hz, 1H), 8.05-7.96 (m, 2H), 7.90-7.82 (m, 1H), 7.73-7.66 (m, 1H), 7.65-7.55 (m, 3H), 7.55-7.40 (m, 2H), 7.34-7.24 (m, 1H), 6.34 (q, J=7.2 Hz, 1H), 5.43 (s, 2H), 2.86-2.71 (m, 2H), 1.98 (d, J=7.2 Hz, 3H), 1.27-1.19 (m, 1H), 0.48-0.35 (m, 2H), 0.25-0.23 (m, 2H). ¹⁹F NMR (376 MHz, DMSO-d₆) δ −74.45, −129.30. LC-MS: m/z 648.5 (M+H)⁺.

### 4-{4-[(5-chloro-2-pyridyl)methyl]-2,2-difluoro-3-oxo-3,4-dihydro-2H-1,4-benzoxazin-7-yl}-1-[(S)-1-(5-cyano-2-pyridyl)ethyl]-6-(cyclopropylmethyl)-1,2,7-triaza-1H-indene-5-carboxamide and 4-{4-[(5-chloro-2-pyridyl)methyl]-2,2-difluoro-3-oxo-3,4-dihydro-2H-1,4-benzoxazin-7-yl}-1-[(R)-1-(5-cyano-2-pyridyl)ethyl]-6-(cyclopropylmethyl)-1,2,7-triaza-1H-indene-5-carboxamide (Compound 235 and Compound 236)

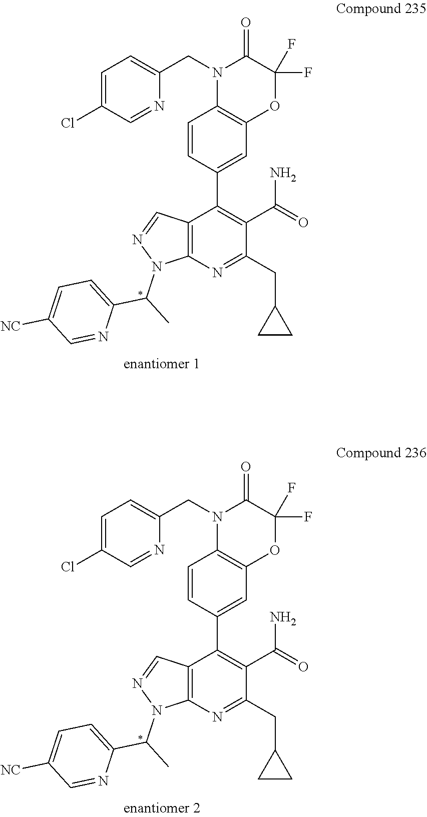 [SMILES: CC(c1ccc(C#N)cn1)n1ncc2c(-c3ccc4c(c3)OC(F)(F)C(=O)N4Cc3ccc(Cl)cn3)c(C(N)=O)c(CC3CC3)nc21 ; CC(c1ccc(C#N)cn1)n1ncc2c(-c3ccc4c(c3)OC(F)(F)C(=O)N4Cc3ccc(Cl)cn3)c(C(N)=O)c(CC3CC3)nc21]

4-{4-[(5-chloro-2-pyridyl)methyl]-2,2-difluoro-3-oxo-3,4-dihydro-2H-1,4-benzoxazin-7-yl}-1-[1-(5-cyano-2-pyridyl)ethyl]-6-(cyclopropylmethyl)-1,2,7-triaza-1H-indene-5-carboxamide which was synthesized following the similar route of Example 57, using 6-(1-hydroxyethyl) nicotinonitrile in step A (45.0 mg) was purified by SFC (system: Waters SFC 17; Column name: DAICEL CHIRALCEL OX; Column size: (250 mm*30 mm, 10 μm); mobile phase A: Supercritical CO₂; mobile phase B: EtOH (0.1% NH₃·H₂O); Gradient: A/B=50/50; Flow rate: 80 mL/min; Column Temp: RT) to afford 4-{4-[(5-chloro-2-pyridyl)methyl]-2,2-difluoro-3-oxo-3,4-dihydro-2H-1,4-benzoxazin-7-yl}-1-[(S)-1-(5-cyano-2-pyridyl)ethyl]-6-(cyclopropylmethyl)-1,2,7-triaza-1H-indene-5-carboxamide (17.33 mg) and 4-{4-[(5-chloro-2-pyridyl)methyl]-2,2-difluoro-3-oxo-3,4-dihydro-2H-1,4-benzoxazin-7-yl}-1-[(R)-1-(5-cyano-2-pyridyl)ethyl]-6-(cyclopropylmethyl)-1,2,7-triaza-1H-indene-5-carboxamide (15.90 mg).

Compound 235, enantiomer 1: Rt=1.11 min. ¹H NMR (400 MHz, DMSO-d₆) δ 8.97 (d, J=1.2 Hz, 1H), 8.59 (d, J=2.0 Hz, 1H), 8.26 (dd, J=8.4, 2.0 Hz, 1H), 8.03-7.97 (m, 2H), 7.89-7.82 (m, 1H), 7.64-7.56 (m, 3H), 7.52-7.43 (m, 2H), 7.36 (d, J=8.4 Hz, 1H), 6.38 (d, J=7.2 Hz, 1H), 5.43 (s, 2H), 2.82-2.68 (m, 2H), 2.00 (d, J=7.2 Hz, 3H), 1.30-1.21 (m, 1H), 0.45-0.33 (m, 2H), 0.26-0.15 (m, 2H). ¹⁹F NMR (376 MHz, DMSO-d₆) δ −74.44. LC-MS: m/z 655.1 (M+H)⁺.

Compound 236, enantiomer 2: Rt=1.44 min. ¹H NMR (400 MHz, DMSO-d₆) δ 8.97 (d, J=1.2 Hz, 1H), 8.59 (d, J=2.0 Hz, 1H), 8.26 (dd, J=8.4, 2.0 Hz, 1H), 8.03-7.97 (m, 2H), 7.89-7.82 (m, 1H), 7.64-7.56 (m, 3H), 7.52-7.43 (m, 2H), 7.36 (d, J=8.4 Hz, 1H), 6.38 (d, J=7.2 Hz, 1H), 5.43 (s, 2H), 2.82-2.68 (m, 2H), 2.00 (d, J=7.2 Hz, 3H), 1.30-1.21 (m, 1H), 0.45-0.33 (m, 2H), 0.26-0.15 (m, 2H). ¹⁹F NMR (376 MHz, DMSO-d₆) δ −74.44. LC-MS: m/z 655.1 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[1-(4-mesyl-2-pyridyl)ethyl]-1,2,7-triaza-1H-indene-5-carboxamide (Compound 237)

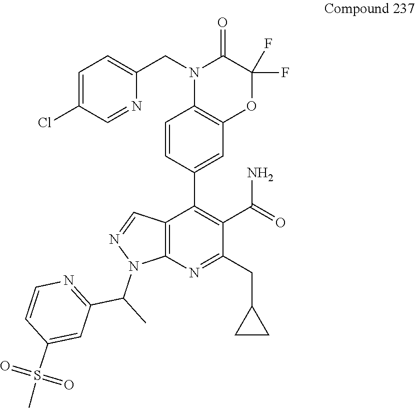 [SMILES: CC(c1cc(S(C)(=O)=O)ccn1)n1ncc2c(-c3ccc4c(c3)OC(F)(F)C(=O)N4Cc3ccc(Cl)cn3)c(C(N)=O)c(CC3CC3)nc21]

Compound 237 was synthesized following the similar route of Example 57, using 1-(4-(methylsulfonyl)pyridin-2-yl) ethan-1-ol in step A.

¹H NMR (400 MHz, DMSO-d₆) δ 8.85 (d, J=5.2 Hz, 1H), 8.59 (d, J=2.4 Hz, 1H), 8.03-7.98 (m, 2H), 7.88-7.83 (m, 2H), 7.81 (s, 1H), 7.63-7.57 (m, 3H), 7.52-7.43 (m, 2H), 6.46 (q, J=7.2 Hz, 1H), 5.43 (s, 2H), 3.31 (s, 3H), 2.77 (d, J=6.8 Hz, 2H), 2.02 (d, J=7.2 Hz, 3H), 1.28-1.18 (m, 1H), 0.45-0.35 (m, 2H), 0.27-0.16 (m, 2H). ¹⁹F NMR (376 MHz, DMSO-d₆) δ −74.43. LC-MS: m/z 708.1 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[1-(5-fluoro-2-pyrimidinyl)ethyl]-1,2,7-triaza-1H-indene-5-carboxamide (Compound 238)

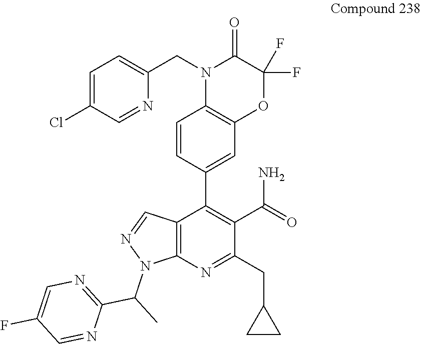 [SMILES: CC(c1ncc(F)cn1)n1ncc2c(-c3ccc4c(c3)OC(F)(F)C(=O)N4Cc3ccc(Cl)cn3)c(C(N)=O)c(CC3CC3)nc21]

Compound 238 was synthesized following the similar route of Example 57, using 1-(5-fluoropyrimidin-2-yl) ethan-1-ol in step A.

¹H NMR (400 MHz, DMSO-d₆) δ 8.85 (s, 2H), 8.59 (d, J=2.0 Hz, 1H), 8.01 (dd, J=8.4, 2.4 Hz, 1H), 7.94 (s, 1H), 7.86 (s, 1H), 7.63-7.57 (m, 3H), 7.52-7.43 (m, 2H), 6.43-6.34 (m, 1H), 5.43 (s, 2H), 2.78-2.65 (m, 2H), 2.01 (d, J=7.2 Hz, 3H), 1.20-1.10 (m, 1H), 0.40-0.30 (m, 2H), 0.23-0.15 (m, 2H). ¹⁹F NMR (376 MHz, DMSO-d₆) δ −74.41, −140.00 LC-MS: m/z 649.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-(5-fluoro-2-pyrimidinyl)ethyl]-1,2,7-triaza-1H-indene-5-carboxamide and 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(R)-1-(5-fluoro-2-pyrimidinyl)ethyl]-1,2,7-triaza-1H-indene-5-carboxamide (Compound 239 and Compound 240)

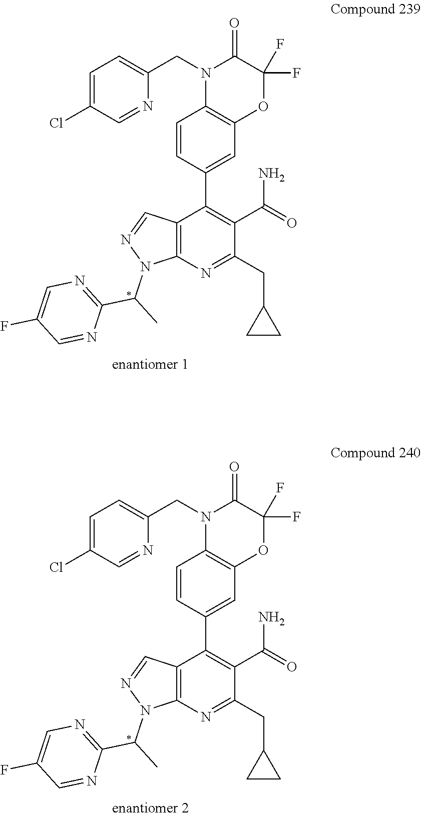 [SMILES: CC(c1ncc(F)cn1)n1ncc2c(-c3ccc4c(c3)OC(F)(F)C(=O)N4Cc3ccc(Cl)cn3)c(C(N)=O)c(CC3CC3)nc21 ; CC(c1ncc(F)cn1)n1ncc2c(-c3ccc4c(c3)OC(F)(F)C(=O)N4Cc3ccc(Cl)cn3)c(C(N)=O)c(CC3CC3)nc21]

4-{4-[(5-chloro-2-pyridyl)methyl]-2,2-difluoro-3-oxo-3,4-dihydro-2H-1,4-benzoxazin-7-yl}-6-(cyclopropylmethyl)-1-[1-(5-fluoro-2-pyrimidinyl)ethyl]-1,2,7-triaza-1H-indene-5-carboxamide (170.0 mg) was purified by SFC (system: system: Waters SFC 80Q; Column name: DAICEL CHIRALCEL OD; Column size: (250 mm*30 mm, 10 μm); mobile phase A: Supercritical CO₂; mobile phase B: EtOH (+0.1% NH₃·H₂O); Gradient: A/B=60/40; Flow rate: 80 mL/min; Column Temp: RT) to afford 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-(5-fluoro-2-pyrimidinyl)ethyl]-1,2,7-triaza-1H-indene-5-carboxamide (56.88 mg) and 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(R)-1-(5-fluoro-2-pyrimidinyl)ethyl]-1,2,7-triaza-1H-indene-5-carboxamide (55.06 mg).

Compound 239, enantiomer 1: Rt=3.05 min. ¹H NMR (400 MHz, DMSO-d₆) δ 8.85 (s, 1H), 8.59 (d, J=2.4 Hz, 1H), 8.01 (dd, J=8.8, 2.4 Hz, 1H), 7.94 (s, 1H), 7.86 (s, 1H), 7.63-7.57 (m, 3H), 7.52-7.43 (m, 2H), 6.43-6.34 (m, 1H), 5.43 (s, 2H), 2.78-2.65 (m, 2H), 2.01 (d, J=6.8 Hz, 3H), 1.20-1.10 (m, 1H), 0.40-0.30 (m, 2H), 0.23-0.15 (m, 2H). ¹⁹F NMR (376 MHz, DMSO-d₆) δ −74.41, −140.01. LC-MS: m/z 649.2 (M+H)⁺.

Compound 240, enantiomer 2: Rt=3.26 min. ¹H NMR (400 MHz, DMSO-d₆) δ 8.85 (s, 1H), 8.59 (d, J=2.4 Hz, 1H), 8.01 (dd, J=8.4, 2.4 Hz, 1H), 7.94 (s, 1H), 7.86 (s, 1H), 7.63-7.57 (m, 3H), 7.52-7.43 (m, 2H), 6.43-6.34 (m, 1H), 5.43 (s, 2H), 2.78-2.65 (m, 2H), 2.01 (d, J=7.2 Hz, 3H), 1.20-1.10 (m, 1H), 0.40-0.30 (m, 2H), 0.23-0.15 (m, 2H). ¹⁹F NMR (376 MHz, DMSO-d₆) δ −74.41, −140.02. LC-MS: m/z 649.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[1-(1-methyl-6-oxo-1,6-dihydro-3-pyridyl)ethyl]-1,2,7-triaza-1H-indene-5-carboxamide (Compound 241)

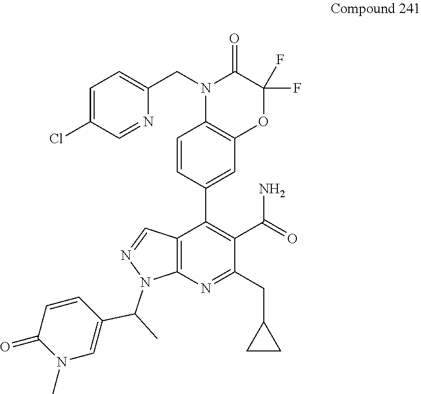 [SMILES: CC(c1ccc(=O)n(C)c1)n1ncc2c(-c3ccc4c(c3)OC(F)(F)C(=O)N4Cc3ccc(Cl)cn3)c(C(N)=O)c(CC3CC3)nc21]

Compound 241 was synthesized following the similar route of Example 57, using 5-(1-hydroxyethyl)-1-methylpyridin-2 (1H)-one in step A.

¹H NMR (400 MHz, DMSO-d₆) δ 8.59 (d, J=2.4 Hz, 1H), 8.02 (dd, J=8.8, 2.8 Hz, 1H), 7.94 (s, 1H), 7.88 (d, J=2.4 Hz, 1H), 7.85-7.82 (m, 1H), 7.66-7.57 (m, 3H), 7.54-7.41 (m, 3H), 6.33 (d, J=9.2 Hz, 1H), 6.11-6.00 (m, 1H), 5.43 (s, 2H), 3.40 (s, 3H), 2.93-2.73 (m, 2H), 1.87 (d, J=7.2 Hz, 3H), 1.41-1.26 (m, 1H), 0.57-0.43 (m, 2H), 0.40-0.25 (m, 2H). ¹⁹F NMR (376 MHz, DMSO-d₆) δ −74.44. LC-MS: m/z 660.2 (M+H)⁺.

The following compounds were synthesized following the similar route of Previous Examples with appropriate starting materials.

### 4-{4-[(5-chloro-2-pyridyl)methyl]-2,2-dimethyl-3-oxo-3,4-dihydro-2H-1,4-benzoxazin-7-yl}-6-(cyclopropylmethyl)-1-[1-(p-mesylphenyl)ethyl]-1,2,7-triaza-1H-indene-5-carboxamide (Compound 242)

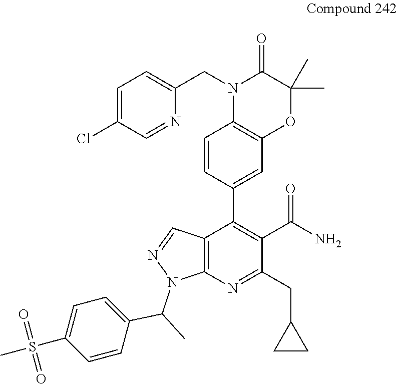

¹H NMR (400 MHz, DMSO-d₆) δ 8.60 (d, J=2.4 Hz, 1H), 8.01 (s, 1H), 7.96 (dd, J=2.4, 8.4 Hz, 1H), 7.90-7.85 (m, 2H), 7.77 (d, J=1.6 Hz, 1H), 7.66-7.60 (m, 2H), 7.58-7.49 (m, 1H), 7.41 (d, J=8.4 Hz, 1H), 7.29 (d, J=2.0 Hz, 1H), 7.25 (dd, J=2.0, 8.0 Hz, 1H), 7.14 (d, J=8.4 Hz, 1H), 6.38 (q, J=6.8 Hz, 1H), 5.26 (s, 2H), 3.17 (s, 3H), 2.85-2.72 (m, 2H), 2.00 (d, J=7.2 Hz, 3H), 1.56-1.49 (m, 6H), 1.33-1.20 (m, 1H), 0.49-0.39 (m, 2H), 0.32-0.23 (m, 2H). LC-MS: m/z 699.4 (M+H)⁺.

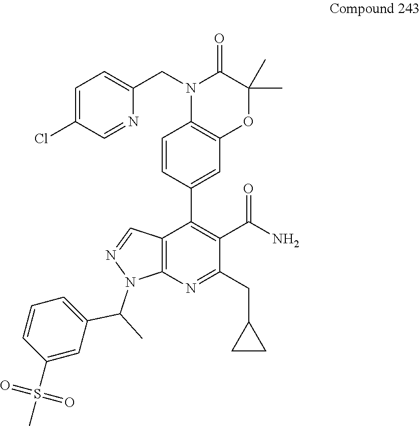 [SMILES: CC(c1cccc(S(C)(=O)=O)c1)n1ncc2c(-c3ccc4c(c3)OC(C)(C)C(=O)N4Cc3ccc(Cl)cn3)c(C(N)=O)c(CC3CC3)nc21]

¹H NMR (400 MHz, DMSO-d₆) δ 8.58 (d, J=2.4 Hz, 1H), 8.02 (s, 1H), 7.99-7.97 (m, 1H), 7.94 (dd, J=8.4, 2.4 Hz, 1H), 7.83 (d, J=7.6 Hz, 1H), 7.78-7.71 (m, 2H), 7.64-7.57 (m, 1H), 7.55-7.50 (m, 1H), 7.40 (d, J=8.4 Hz, 1H), 7.28 (d, J=2.0 Hz, 1H), 7.26-7.22 (m, 1H), 7.13 (d, J=8.4 Hz, 1H), 6.40 (q, J=7.2 Hz, 1H), 5.25 (s, 2H), 3.19 (s, 3H), 2.84-2.72 (m, 2H), 2.00 (d, J=7.2 Hz, 3H), 1.51 (s, 6H), 1.31-1.24 (m, 1H), 0.49-0.39 (m, 2H), 0.33-0.21 (m, 2H). LC-MS: m/z 699.4 (M+H)⁺.

### 4-{4-[(5-chloro-2-pyridyl)methyl]-2,2-dimethyl-3-oxo-3,4-dihydro-2H-1,4-benzoxazin-7-yl}-6-(cyclopropylmethyl)-1-[1-(5-fluoro-2-pyridyl)ethyl]-1,2,7-triaza-1H-indene-5-carboxamide (Compound 244)

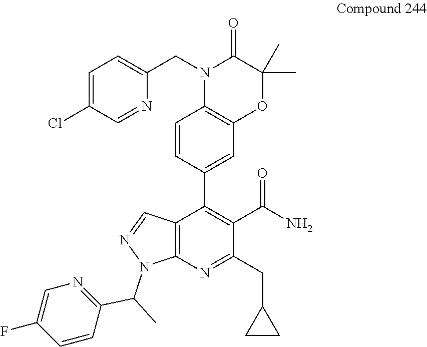 [SMILES: CC(c1ccc(F)cn1)n1ncc2c(-c3ccc4c(c3)OC(C)(C)C(=O)N4Cc3ccc(Cl)cn3)c(C(N)=O)c(CC3CC3)nc21]

¹H NMR (400 MHz, DMSO-d₆) δ 8.59 (d, J=2.4 Hz, 1H), 8.50 (d, J=3.2 Hz, 1H), 7.97 (s, 1H), 7.95 (dd, J=8.4, 2.4 Hz, 1H), 7.77 (s, 1H), 7.66 (td, J=8.8, 3.2 Hz, 1H), 7.52 (s, 1H), 7.40 (d, J=8.4 Hz, 1H), 7.29 (d, J=2.0 Hz, 1H), 7.23-7.27 (m, 2H), 7.14 (d, J=8.4 Hz, 1H), 5.25 (s, 2H), 2.72-2.80 (m, 2H), 1.97 (d, J=7.2 Hz, 3H), 1.52 (d, J=1.2 Hz, 6H), 1.23 (br d, J=3.2 Hz, 1H), 0.37-0.43 (m, 2H), 0.21-0.26 (m, 2H). ¹⁹F NMR (376 MHz, DMSO-d₆) δ −129.38. LC-MS: m/z 640.2 (M+H)⁺.

### 4-{4-[(5-chloro-2-pyridyl)methyl]-2,2-dimethyl-3-oxo-3,4-dihydro-2H-1,4-benzoxazin-7-yl}-6-(cyclopropylmethyl)-1-[(S)-1-(5-fluoro-2-pyridyl)ethyl]-1,2,7-triaza-1H-indene-5-carboxamide and 4-{4-[(5-chloro-2-pyridyl)methyl]-2,2-dimethyl-3-oxo-3,4-dihydro-2H-1,4-benzoxazin-7-yl}-6-(cyclopropylmethyl)-1-[(R)-1-(5-fluoro-2-pyridyl)ethyl]-1,2,7-triaza-1H-indene-5-carboxamide (Compound 245 and Compound 246)

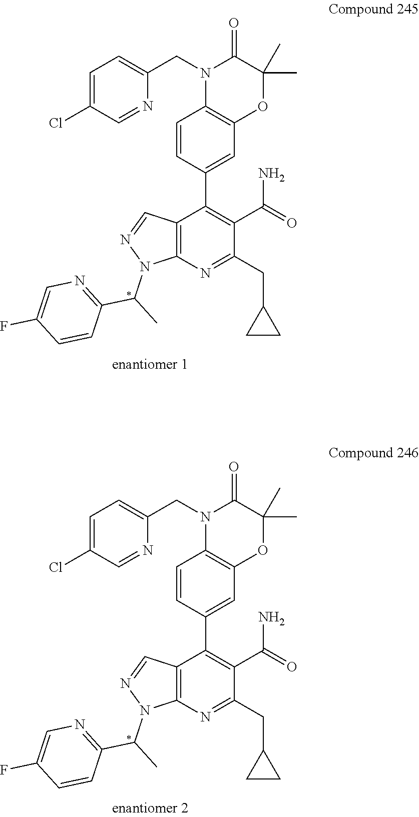 [SMILES: CC(c1ccc(F)cn1)n1ncc2c(-c3ccc4c(c3)OC(C)(C)C(=O)N4Cc3ccc(Cl)cn3)c(C(N)=O)c(CC3CC3)nc21 ; CC(c1ccc(F)cn1)n1ncc2c(-c3ccc4c(c3)OC(C)(C)C(=O)N4Cc3ccc(Cl)cn3)c(C(N)=O)c(CC3CC3)nc21]

4-{4-[(5-chloro-2-pyridyl)methyl]-2,2-dimethyl-3-oxo-3,4-dihydro-2H-1,4-benzoxazin-7-yl}-6-(cyclopropylmethyl)-1-[1-(5-fluoro-2-pyridyl)ethyl]-1,2,7-triaza-1H-indene-5-carboxamide (210 mg) was purified by SFC (system: Waters SFC 150; Column name: Daicel ChiralPak IG; Column size: (250 mm*30 mm, 10 μm); mobile phase A: Supercritical CO₂; mobile phase B: i-PrOH (+0.1% NH₃H₂O); Gradient: A/B=60/40; Flow rate: 80 mL/min; Column Temp: RT) to afford 4-{4-[(5-chloro-2-pyridyl)methyl]-2,2-dimethyl-3-oxo-3,4-dihydro-2H-1,4-benzoxazin-7-yl}-6-(cyclopropylmethyl)-1-[(S)-1-(5-fluoro-2-pyridyl)ethyl]-1,2,7-triaza-1H-indene-5-carboxamide (91 mg) and 4-{4-[(5-chloro-2-pyridyl)methyl]-2,2-dimethyl-3-oxo-3,4-dihydro-2H-1,4-benzoxazin-7-yl}-6-(cyclopropylmethyl)-1-[(R)-1-(5-fluoro-2-pyridyl)ethyl]-1,2,7-triaza-1H-indene-5-carboxamide (15 mg).

Compound 245, enantiomer 1: Rt=1.59 min. ¹H NMR (400 MHz, DMSO-d₆) δ 8.59 (d, J=2.4 Hz, 1H), 8.50 (d, J=2.8 Hz, 1H), 7.97 (s, 1H), 7.95 (dd, J=8.4, 2.4 Hz, 1H), 7.77 (s, 1H), 7.66 (td, J=8.8, 2.8 Hz, 1H), 7.52 (s, 1H), 7.40 (d, J=8.4 Hz, 1H), 7.29 (d, J=1.6 Hz, 1H), 7.23-7.27 (m, 2H), 7.14 (d, J=8.4 Hz, 1H), 6.32 (q, J=7.2 Hz, 1H), 5.25 (s, 2H), 2.76 (t, J=6.4 Hz, 2H), 1.97 (d, J=7.2 Hz, 3H), 1.52 (s, 6H), 1.20-1.26 (m, 1H), 0.37-0.43 (m, 2H), 0.24 (br d, J=4.4 Hz, 2H). ¹⁹F NMR (376 MHz, DMSO-d₆) δ −129.39. LC-MS: m/z 640.2 (M+H)⁺.

Compound 246, enantiomer 2: Rt=1.68 min. ¹H NMR (400 MHz, DMSO-d₆) δ 8.59 (d, J=2.4 Hz, 1H) 8.51 (d, J=2.8 Hz, 1H) 7.98 (s, 1H) 7.95 (dd, J=8.4, 2.4 Hz, 1H) 7.78 (br s, 1H) 7.67 (td, J=8.8, 2.8 Hz, 1H) 7.53 (br s, 1H) 7.41 (d, J=8.4 Hz, 1H) 7.30 (d, J=2.0 Hz, 1H) 7.23-7.28 (m, 2H) 7.14 (d, J=8.4 Hz, 1H) 6.33 (q, J=7.2 Hz, 1H) 5.26 (s, 2H) 2.77 (t, J=6.4 Hz, 2H) 1.98 (d, J=7.2 Hz, 3H) 1.52 (s, 6H) 1.24 (br s, 1H) 0.37-0.44 (m, 2H) 0.24 (br d, J=4.8 Hz, 2H). ¹⁹F NMR (376 MHz, DMSO-d₆) δ −129.37. LC-MS: m/z 640.2 (M+H)⁺.

### 4-{4-[(5-cyano-2-pyridyl)methyl]-2,2-dimethyl-3-oxo-3,4-dihydro-2H-1,4-benzoxazin-7-yl}-6-(cyclopropylmethyl)-1-[(S)-1-(5-fluoro-2-pyridyl)ethyl]-1,2,7-triaza-1H-indene-5-carboxamide (Compound 247)

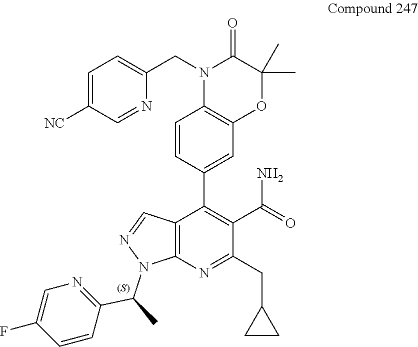

¹H NMR (400 MHz, CD₃OD) δ 8.86 (d, J=1.2 Hz, 1H), 8.38 (d, J=3.2 Hz, 1H), 8.16 (dd, J=2.0, 8.4 Hz, 1H), 7.94 (s, 1H), 7.56-7.46 (m, 2H), 7.32 (d, J=1.6 Hz, 1H), 7.27-7.23 (m, 2H), 7.09 (d, J=8.4 Hz, 1H), 6.41 (q, J=6.8 Hz, 1H), 5.39 (s, 2H), 2.87 (dd, J=4.0, 6.8 Hz, 2H), 2.04 (d, J=7.2 Hz, 3H), 1.58 (s, 6H), 0.90 (t, J=6.8 Hz, 1H), 0.49-0.43 (m, 2H), 0.30-0.25 (m, 2H). ¹⁹F NMR (376 MHz, CD₃OD) δ −130.97. LC-MS: m/z 631.2 (M+H)⁺.

### 4-{4-[(5-chloro-2-pyrimidinyl)methyl]-2,2-dimethyl-3-oxo-3,4-dihydro-2H-1,4-benzoxazin-7-yl}-6-(cyclopropylmethyl)-1-[(S)-1-(5-fluoro-2-pyridyl)ethyl]-1,2,7-triaza-1H-indene-5-carboxamide and 4-{4-[(5-chloro-2-pyrimidinyl)methyl]-2,2-dimethyl-3-oxo-3,4-dihydro-2H-1,4-benzoxazin-7-yl}-6-(cyclopropylmethyl)-1-[(R)-1-(5-fluoro-2-pyridyl)ethyl]-1,2,7-triaza-1H-indene-5-carboxamide (Compound 248 and Compound 249)

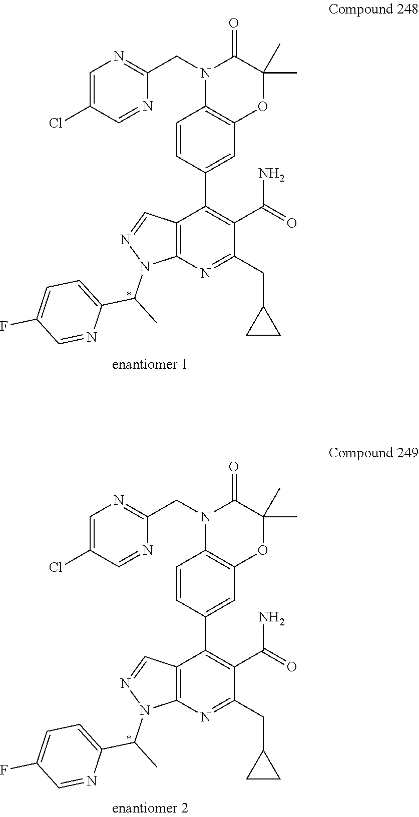 [SMILES: CC(c1ccc(F)cn1)n1ncc2c(-c3ccc4c(c3)OC(C)(C)C(=O)N4Cc3ncc(Cl)cn3)c(C(N)=O)c(CC3CC3)nc21 ; CC(c1ccc(F)cn1)n1ncc2c(-c3ccc4c(c3)OC(C)(C)C(=O)N4Cc3ncc(Cl)cn3)c(C(N)=O)c(CC3CC3)nc21]

The racemic compound (170.0 mg) was separated by SFC (system: Waters SFC 21; Column name: DAICEL CHIRALCEL OD; Column size: (250 mm*30 mm, 10 μm); mobile phase A: Supercritical CO₂; mobile phase B: i-PrOH (+0.1% NH₃·H₂O)]; Gradient: A/B=60/40; Flow rate: 80 mL/min; Column Temp: RT) to afford 4-{4-[(5-chloro-2-pyrimidinyl)methyl]-2,2-dimethyl-3-oxo-3,4-dihydro-2H-1,4-benzoxazin-7-yl}-6-(cyclopropylmethyl)-1-[(S)-1-(5-fluoro-2-pyridyl)ethyl]-1,2,7-triaza-1H-indene-5-carboxamide (53.0 mg) and 4-{4-[(5-chloro-2-pyrimidinyl)methyl]-2,2-dimethyl-3-oxo-3,4-dihydro-2H-1,4-benzoxazin-7-yl}-6-(cyclopropylmethyl)-1-[(R)-1-(5-fluoro-2-pyridyl)ethyl]-1,2,7-triaza-1H-indene-5-carboxamide (9.0 mg).

Compound 248, enantiomer 1: Rt=3.29 min. ¹H NMR (400 MHz, CD₃OD) δ 8.77 (s, 2H), 8.38 (d, J=2.4 Hz, 1H), 7.95 (s, 1H), 7.57-7.42 (m, 1H), 7.33 (d, J=1.6 Hz, 1H), 7.29-7.18 (m, 2H), 6.99 (d, J=8.4 Hz, 1H), 6.41 (q, J=6.8 Hz, 1H), 5.39 (s, 2H), 2.91-2.82 (m, 2H), 2.04 (d, J=7.2 Hz, 3H), 1.58 (s, 6H), 1.33-1.25 (m, 1H), 0.50-0.40 (m, 2H), 0.32-0.24 (m, 2H). ¹⁹F NMR (376 MHz, CD₃OD) δ −130.98. LC-MS: m/z 641.3 (M+H)⁺.

Compound 249, enantiomer 2: Rt=3.56 min. ¹H NMR (400 MHz, CD₃OD) δ 8.77 (s, 2H), 8.38 (d, J=2.4 Hz, 1H), 7.95 (s, 1H), 7.57-7.42 (m, 1H), 7.32 (d, J=2.0 Hz, 1H), 7.29-7.18 (m, 2H), 6.99 (d, J=8.4 Hz, 1H), 6.41 (q, J=6.8 Hz, 1H), 5.39 (s, 2H), 2.91-2.82 (m, 2H), 2.04 (d, J=7.6 Hz, 3H), 1.58 (s, 6H), 1.33-1.25 (m, 1H), 0.50-0.40 (m, 2H), 0.32-0.24 (m, 2H). ¹⁹F NMR (376 MHz, CD₃OD) δ −130.96. LC-MS: m/z 641.3 (M+H)⁺.

### 4-{4-[(5-chloro-2-pyridyl)methyl]-2,2-dimethyl-3-oxo-3,4-dihydro-2H-1,4-benzoxazin-7-yl}-6-(cyclopropylmethyl)-1-[1-(6-difluoromethoxy-2-pyridyl)ethyl]-1,2,7-triaza-1H-indene-5-carboxamide (Compound 250)

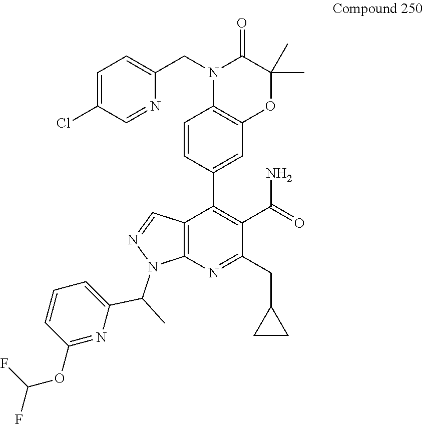 [SMILES: CC(c1cccc(OC(F)F)n1)n1ncc2c(-c3ccc4c(c3)OC(C)(C)C(=O)N4Cc3ccc(Cl)cn3)c(C(N)=O)c(CC3CC3)nc21]

¹H NMR (400 MHz, DMSO-d₆) δ 8.59 (d, J=2.4 Hz, 1H), 8.50 (s, 1H), 7.96 (dd, J=2.4, 8.4 Hz, 1H), 7.92-7.85 (m, 1H), 7.74 (d, J=5.2 Hz, 1H), 7.48 (s, 1H), 7.41 (d, J=8.4 Hz, 1H), 7.30 (d, J=2.0 Hz, 1H), 7.29-7.24 (m, 1H), 7.11 (d, J=8.4 Hz, 1H), 7.01 (d, J=8.4 Hz, 1H), 6.97 (d, J=7.6 Hz, 1H), 6.00-5.94 (m, 1H), 5.26 (s, 2H), 2.76 (d, J=6.8 Hz, 2H), 2.04-1.94 (m, 4H), 1.53 (s, 6H), 0.90-0.83 (m, 1H), 0.49-0.41 (m, 2H), 0.26-0.23 (m, 2H). ¹⁹F NMR (376 MHz, METHANOL-d₄) δ −87.26. LC-MS: m/z 688.4 (M+H)⁺.

### 4-{4-[(5-chloro-2-pyridyl)methyl]-2,2-dimethyl-3-oxo-3,4-dihydro-2H-1,4-benzoxazin-7-yl}-6-(cyclopropylmethyl)-1-[1-(6-difluoromethoxy-3-pyridyl)ethyl]-1,2,7-triaza-1H-indene-5-carboxamide (Compound 251)

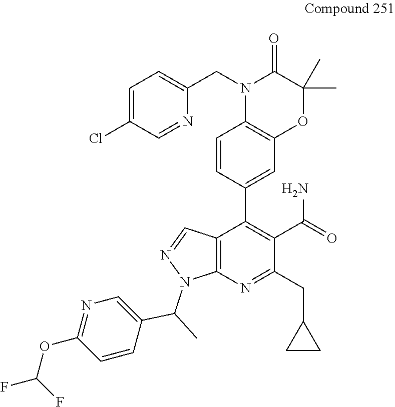 [SMILES: CC(c1ccc(OC(F)F)nc1)n1ncc2c(-c3ccc4c(c3)OC(C)(C)C(=O)N4Cc3ccc(Cl)cn3)c(C(N)=O)c(CC3CC3)nc21]

¹H NMR (400 MHz, DMSO-d₆) δ 8.58 (d, J=2.4 Hz, 1H), 8.30 (d, J=2.4 Hz, 1H), 7.97 (s, 1H), 7.96 (t, J=2.4 Hz, 1H), 7.94 (t, J=2.8 Hz, 1H), 7.75 (s, 1H), 7.52 (d, J=2.0 Hz, 1H), 7.45 (s, 1H), 7.40 (d, J=8.4 Hz, 1H), 7.27 (d, J=2.0 Hz, 1H), 7.21-7.25 (m, 1H), 7.12 (d, J=8.4 Hz, 1H), 7.04 (d, J=8.4 Hz, 1H), 6.29-6.37 (m, 1H), 5.25 (s, 2H), 2.73-2.85 (m, 2H), 1.98 (d, J=7.2 Hz, 3H), 1.51 (s, 6H), 1.25-1.29 (m, 1H), 0.42-0.48 (m, 2H), 0.25-0.31 (m, 2H). ¹⁹F NMR (376 MHz, DMSO-d₆) δ −87.33. LC-MS: m/z 688.1 (M+H)⁺.

### 4-{4-[(5-chloro-2-pyridyl)methyl]-2,2-dimethyl-3-oxo-3,4-dihydro-2H-1,4-benzoxazin-7-yl}-6-(cyclopropylmethyl)-1-[1-(2-difluoromethoxy-4-pyridyl)ethyl]-1,2,7-triaza-1H-indene-5-carboxamide (Compound 252)

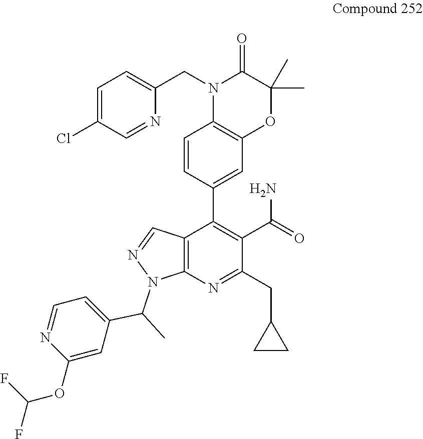 [SMILES: CC(c1ccnc(OC(F)F)c1)n1ncc2c(-c3ccc4c(c3)OC(C)(C)C(=O)N4Cc3ccc(Cl)cn3)c(C(N)=O)c(CC3CC3)nc21]

¹H NMR (400 MHz, DMSO-d₆) δ 8.58 (d, J=2.4 Hz, 1H), 8.18 (d, J=5.2 Hz, 1H), 8.02 (s, 1H), 7.94 (dd, J=8.4, 2.4 Hz, 1H), 7.65 (s, 1H), 7.65 (t, J=72.8 Hz, 1H), 7.53 (s, 1H), 7.40 (d, J=8.4 Hz, 1H), 7.31-7.22 (m, 2H), 7.16-7.10 (m, 2H), 6.97 (s, 1H), 6.36-6.26 (m, 1H), 5.25 (s, 2H), 2.85-2.70 (m, 2H), 1.97 (d, J=7.2 Hz, 3H), 1.52 (s, 6H), 0.89-0.81 (m, 1H), 0.45-0.35 (m, 2H), 0.30-0.20 (m, 2H). ¹⁹F NMR (376 MHz, DMSO-d₆) δ −87.40, −129.34. LC-MS: m/z 688.2 (M+H)⁺.

### 6-(cyclopropylmethyl)-4-{2,2-difluoro-4-[(5-fluoro-2-pyrimidinyl)methyl]-3-oxo-3,4-dihydro-2H-1,4-benzoxazin-7-yl}-1-[1-(5-fluoro-2-pyrimidinyl)ethyl]-1,2,7-triaza-1H-indene-5-carboxamide (Compound 253)

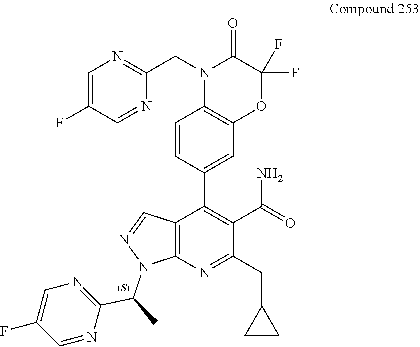

¹H NMR (400 MHz, DMSO-d₆) δ 8.94 (s, 2H), 8.85 (s, 2H), 7.94 (s, 1H), 7.87 (d, J=1.2 Hz, 1H), 7.63 (d, J=1.6 Hz, 1H), 7.60 (d, J=1.2 Hz, 1H), 7.49 (dd, J₁=1.6 Hz, J₂=8.8 Hz, 1H), 7.43 (d, J=8.8 Hz, 1H), 6.39 (dd, J₁=7.2 Hz, J₂=14.4 Hz, 1H), 5.51 (s, 2H), 2.73 (dd, J₁=4.4 Hz, J₂=6.4 Hz, 2H), 2.01 (d, J=7.2 Hz, 3H), 1.13-1.20 (m, 1H), 0.34-0.37 (m, 2H), 0.16-0.21 (m, 2H). ¹⁹F NMR (376 MHz, DMSO-d₆) δ −74.88, −139.27, −140.00. LC-MS: m/z 634.4 (M+H)⁺.

### 4-{4-[(5-chloro-2-pyridyl)methyl]-2,2-dimethyl-3-oxo-3,4-dihydro-2H-1,4-benzoxazin-7-yl}-6-(cyclopropylmethyl)-1-[1-(5-methyl-1,3,4-oxadiazol-2-yl)ethyl]-1,2,7-triaza-1H-indene-5-carboxamide (Compound 254)

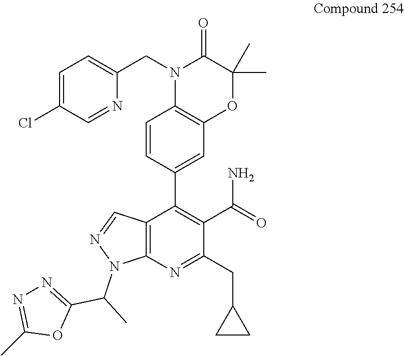

¹H NMR (400 MHz, CD₃OD) δ 8.51 (d, J=2.4 Hz, 1H), 7.95 (s, 1H), 7.83 (dd, J=8.4, 2.4 Hz, 1H), 7.35 (d, J=8.4 Hz, 1H), 7.32 (d, J=2.0 Hz, 1H), 7.25 (dd, J=8.4, 2.0 Hz, 1H), 7.14 (d, J=8.4 Hz, 1H), 6.53 (q, J=7.2 Hz, 1H), 5.29 (s, 2H), 2.89 (d, J=6.8 Hz, 2H), 2.45 (s, 3H), 2.06 (d, J=7.2 Hz, 3H), 1.58 (s, 6H), 1.39-1.32 (m, 1H), 0.54-0.45 (m, 2H), 0.35-0.27 (m, 2H). LC-MS: m/z 627.1 (M+H)⁺.

### 3-chloro-4-{4-[(5-cyano-2-pyridyl)methyl]-2,2-dimethyl-3-oxo-3,4-dihydro-2H-1,4-benzoxazin-7-yl}-6-(cyclopropylmethyl)-1-[(S)-1-(5-fluoro-2-pyridyl)ethyl]-1,2,7-triaza-1H-indene-5-carboxamide (Compound 255)

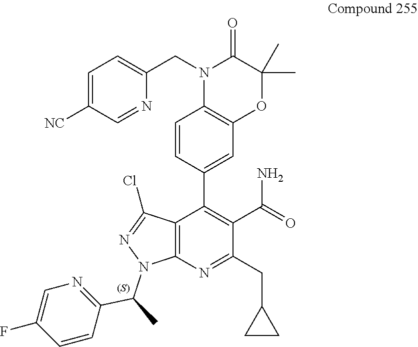

¹H NMR (400 MHz, CD₃OD) δ 8.88 (d, J=1.2 Hz, 1H), 8.39 (s, 1H), 8.17 (dd, J=2.0, 8.0 Hz, 1H), 7.60-7.50 (m, 2H), 7.41-7.33 (m, 1H), 7.14 (s, 1H), 7.05 (s, 2H), 6.41 (q, J=7.2 Hz, 1H), 5.40 (s, 2H), 2.92-2.80 (m, 2H), 2.03 (d, J=7.2 Hz, 3H), 1.59 (s, 6H), 1.33-1.29 (m, 1H), 0.52-0.47 (m, 2H), 0.32-0.26 (m, 2H). ¹⁹F NMR (376 MHz, CD₃OD) δ −130.65. LC-MS: m/z 665.1 (M+H)⁺.

### 4-{3-[(5-cyano-2-pyridyl)methyl]-2-oxo-2,3-dihydro-1,3-benzoxazol-6-yl}-6-(cyclopropylmethyl)-1-[(S)-1-(5-fluoro-2-pyridyl)ethyl]-1,2,7-triaza-1H-indene-5-carboxamide (compound 256)

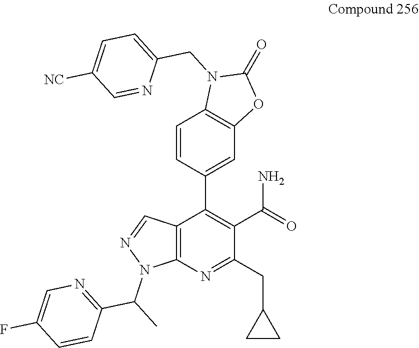 [SMILES: CC(c1ccc(F)cn1)n1ncc2c(-c3ccc4c(c3)oc(=O)n4Cc3ccc(C#N)cn3)c(C(N)=O)c(CC3CC3)nc21]

¹H NMR (400 MHz, CD₃OD) δ 8.88 (d, J=1.6 Hz, 1H), 8.40 (d, J=2.8 Hz, 1H), 8.22 (dd, J=2.0, 8.4 Hz, 1H), 7.97 (s, 1H), 7.70 (d, J=8.4 Hz, 1H), 7.61 (d, J=1.6 Hz, 1H), 7.56-7.46 (m, 2H), 7.31-7.23 (m, 2H), 6.44 (q, J=7.2 Hz, 1H), 5.35 (s, 2H), 2.98-2.83 (m, 2H), 2.06 (d, J=7.2 Hz, 3H), 1.38-1.27 (m, 1H), 0.55-0.43 (m, 2H), 0.34-0.27 (m, 2H). ¹⁹F NMR (376 MHz, CD₃OD) δ −130.93. LC-MS: m/z 589.2 (M+H)⁺.

### 7-{4-[(5-chloro-2-pyrimidinyl)methyl]-2,2-dimethyl-3-oxo-3,4-dihydro-2H-1,4-benzoxazin-7-yl}-5-(cyclopropylmethyl)-3-[1-(p-fluorophenyl)cyclopropyl]-1,3,4-triaza-3H-indene-6-carboxamide (Compound 257)

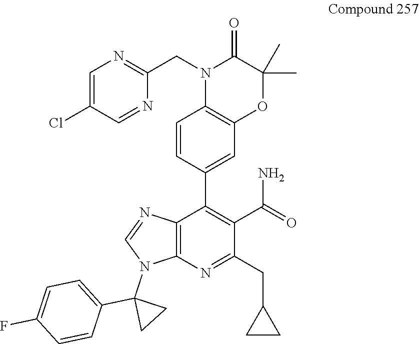 [SMILES: CC1(C)Oc2cc(-c3c(C(N)=O)c(CC4CC4)nc4c3ncn4C3(c4ccc(F)cc4)CC3)ccc2N(Cc2ncc(Cl)cn2)C1=O]

Compound 257 was synthesized following the similar route of Example 51, using 1-(1-(4-fluorophenyl)cyclopropyl)-1H-imidazol-5-amine and 4-((5-chloropyrimidin-2-yl)methyl)-2,2-dimethyl-3-oxo-3,4-dihydro-2H-benzo[b][1,4]oxazine-7-carbaldehyde in step G. ¹H NMR (400 MHz, CD₃OD) δ 8.76 (s, 2H), 8.53 (s, 1H), 7.41-7.50 (m, 2H), 7.30 (d, J=1.6 Hz, 1H), 7.21 (dd, J=8.4, 1.6 Hz, 1H), 6.99 (t, J=8.8 Hz, 2H), 6.92 (d, J=8.4 Hz, 1H), 5.38 (s, 2H), 2.89 (d, J=6.8 Hz, 2H), 1.73-1.81 (m, 2H), 1.62-1.68 (m, 2H), 1.56 (s, 6H), 1.29-1.35 (m, 1H), 0.47-0.54 (m, 2H), 0.30-0.37 (m, 2H). ¹⁹F NMR (376 MHz, CD₃OD) δ −116.92. LC-MS: m/z 652.1 (M+H)⁺.

### 7-{4-[(5-chloro-2-pyridyl)methyl]-2,2-dimethyl-3-oxo-3,4-dihydro-2H-1,4-benzoxazin-7-yl}-5-(cyclopropylmethyl)-3-[(S)-1-(p-fluorophenyl)ethyl]-1,3,4-triaza-3H-indene-6-carboxamide (Compound 258)

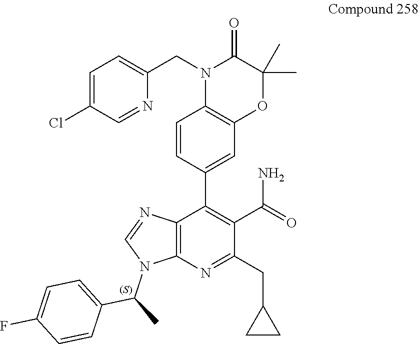

Compound 258 was synthesized following the similar route of Example 51, using(S)-1-(1-(4-fluorophenyl)ethyl)-1H-imidazol-5-amine and 4-[(5-chloropyridin-2-yl)methyl]-2,2-dimethyl-3-oxo-3,4-dihydro-2H-benzo[b][1,4]oxazine-7-carbaldehyde in step G. ¹H NMR (400 MHz, CD₃OD) δ 9.08 (s, 1H), 8.50 (d, J=2.4 Hz, 1H), 7.82 (dd, J=8.4, 2.4 Hz, 1H), 7.52-7.62 (m, 2H), 7.35 (d, J=8.4 Hz, 1H), 7.27 (d, J=2.0 Hz, 1H), 7.17-7.22 (m, 1H), 7.06-7.15 (m, 3H), 6.17 (q, J=7.2 Hz, 1H), 5.30 (s, 2H), 2.82-2.95 (m, 2H), 2.12 (d, J=6.8 Hz, 3H), 1.57 (s, 6H), 1.27-1.31 (m, 1H), 0.45-0.53 (m, 2H), 0.24-0.35 (m, 2H). ¹⁹F NMR (376 MHz, CD₃OD) δ −77.49, −115.75. LC-MS: m/z 639.3 (M+H)⁺.

### 7-{4-[(5-chloro-2-pyridyl)methyl]-2,2-dimethyl-3-oxo-3,4-dihydro-2H-1,4-benzoxazin-7-yl}-5-(cyclopropylmethyl)-3-[1-(5-fluoro-2-pyridyl)cyclopropyl]-1,3,4-triaza-3H-indene-6-carboxamide (Compound 259)

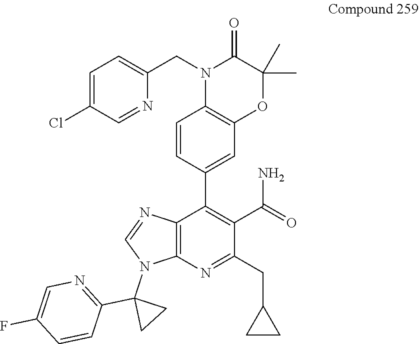 [SMILES: CC1(C)Oc2cc(-c3c(C(N)=O)c(CC4CC4)nc4c3ncn4C3(c4ccc(F)cn4)CC3)ccc2N(Cc2ccc(Cl)cn2)C1=O]

Compound 259 was synthesized following the similar route of Example 51, using 1-(1-(5-fluoropyridin-2-yl)cyclopropyl)-1H-imidazol-5-amine and 4-[(5-chloropyridin-2-yl)methyl]-2,2-dimethyl-3-oxo-3,4-dihydro-2H-benzo[b][1,4]oxazine-7-carbaldehyde in step G. ¹H NMR (400 MHz, CD₃OD) δ 8.44-8.54 (m, 2H), 8.36 (d, J=2.4 Hz, 1H), 7.83 (dd, J=8.4, 2.4 Hz, 1H), 7.32-7.39 (m, 3H), 7.28 (dd, J=8.4, 1.6 Hz, 1H), 7.10 (d, J=8.4 Hz, 1H), 6.72 (dd, J=8.8, 4.4 Hz, 1H), 5.31 (s, 2H), 2.83 (d, J=7.2 Hz, 2H), 1.98 (d, J=2.4 Hz, 2H), 1.75-1.88 (m, 2H), 1.58 (s, 6H), 1.12-1.25 (m, 1H), 0.30-0.47 (m, 2H), 0.22-0.24 (m, 2H). ¹⁹F NMR (376 MHz, CD₃OD) δ −132.94. LC-MS: m/z 652.2 (M+H)⁺.

### 7-{4-[(5-chloro-2-pyrimidinyl)methyl]-2,2-dimethyl-3-oxo-3,4-dihydro-2H-1,4-benzoxazin-7-yl}-5-(cyclopropylmethyl)-3-[1-(5-fluoro-2-pyridyl)cyclopropyl]-1,3,4-triaza-3H-indene-6-carboxamide (Compound 260)

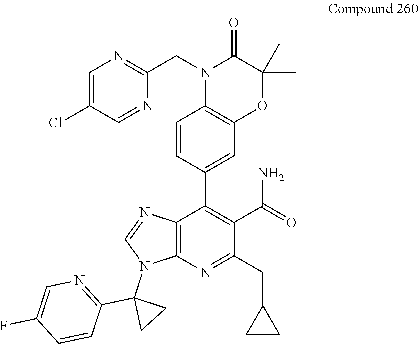 [SMILES: CC1(C)Oc2cc(-c3c(C(N)=O)c(CC4CC4)nc4c3ncn4C3(c4ccc(F)cn4)CC3)ccc2N(Cc2ncc(Cl)cn2)C1=O]

Compound 260 was synthesized following the similar route of Example 51, using 1-(1-(5-fluoropyridin-2-yl)cyclopropyl)-1H-imidazol-5-amine and 4-((5-chloropyrimidin-2-yl)methyl)-2,2-dimethyl-3-oxo-3,4-dihydro-2H-benzo[b][1,4]oxazine-7-carbaldehyde in step G. ¹H NMR (400 MHz, CD₃OD) δ 8.79 (s, 2H), 8.51 (s, 1H), 8.38 (d, J=2.8 Hz, 1H), 7.44-7.35 (m, 2H), 7.29 (dd, J=1.6, 8.4 Hz, 1H), 6.97 (d, J=8.4 Hz, 1H), 6.74 (dd, J=4.0, 8.8 Hz, 1H), 5.41 (s, 2H), 2.85 (d, J=7.2 Hz, 2H), 2.04-1.97 (m, 2H), 1.88-1.79 (m, 2H), 1.60 (s, 6H), 1.28-1.17 (m, 1H), 0.45-0.37 (m, 2H), 0.30-0.23 (m, 2H). ¹⁹F NMR (376 MHz, CD₃OD) δ −132.93. LC-MS: m/z 653.1 (M+H)⁺.

### 7-{4-[(5-chloro-2-pyridyl)methyl]-2,2-dimethyl-3-oxo-3,4-dihydro-2H-1,4-benzoxazin-7-yl}-5-(cyclopropylmethyl)-3-[1-(5-fluoro-2-pyridyl)ethyl]-1,3,4-triaza-3H-indene-6-carboxamide (Compound 261)

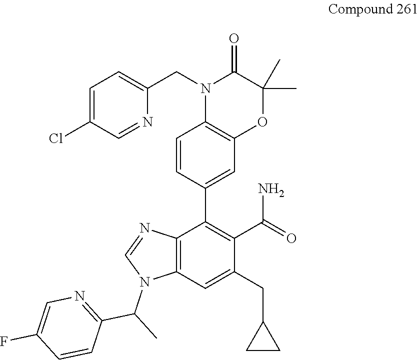 [SMILES: CC(c1ccc(F)cn1)n1cnc2c(-c3ccc4c(c3)OC(C)(C)C(=O)N4Cc3ccc(Cl)cn3)c(C(N)=O)c(CC3CC3)cc21]

Compound 261 was synthesized following the similar route of Example 51, using(S)-1-(1-(5-fluoropyridin-2-yl)ethyl)-1H-imidazol-5-amine and 4-[(5-chloropyridin-2-yl)methyl]-2,2-dimethyl-3-oxo-3,4-dihydro-2H-benzo[b][1,4]oxazine-7-carbaldehyde in step G.

¹H NMR (400 MHz, CD₃OD) δ 8.56 (s, 1H), 8.51 (d, J=2.4 Hz, 1H), 8.42-8.36 (m, 1H), 7.82 (dd, J=2.4, 8.4 Hz, 1H), 7.59-7.55 (m, 2H), 7.33 (d, J=8.4 Hz, 1H), 7.29 (d, J=2.0 Hz, 1H), 7.23 (dd, J=2.0, 8.4 Hz, 1H), 7.08 (d, J=8.4 Hz, 1H), 6.16 (q, J=7.2 Hz, 1H), 5.29 (s, 2H), 2.90-2.75 (m, 2H), 2.06 (d, J=7.2 Hz, 3H), 1.57 (s, 6H), 1.27-1.16 (m, 1H), 0.46-0.39 (m, 2H), 0.29-0.23 (m, 2H). ¹⁹F NMR (376 MHz, CD₃OD) δ −130.31. LC-MS: m/z 640.2 (M+H)⁺

### 7-{4-[(5-chloro-2-pyridyl)methyl]-2,2-difluoro-3-oxo-3,4-dihydro-2H-1,4-benzoxazin-7-yl}-5-(cyclopropylmethyl)-3-[(S)-1-(p-fluorophenyl)ethyl]-1,3,4-triaza-3H-indene-6-carboxamide (compound 262)

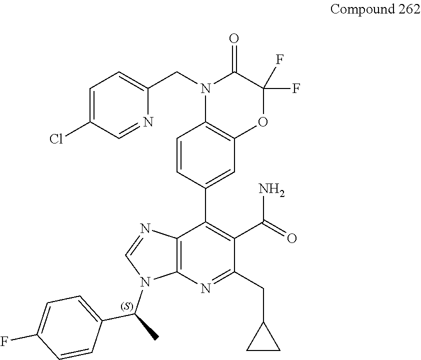

Compound 262 was synthesized following the similar route of Example 51, using(S)-1-(1-(4-fluorophenyl)ethyl)-1H-imidazol-5-amine and 4-((5-chloropyridin-2-yl)methyl)-2,2-difluoro-3-oxo-3,4-dihydro-2H-benzo[b][1,4]oxazine-7-carbaldehyde in step G.

¹H NMR (400 MHz, CD₃OD) δ 8.46-8.53 (m, 2H), 7.85 (dd, J=8.4, 2.4 Hz, 1H), 7.55 (d, J=1.6 Hz, 1H), 7.52 (dd, J=8.8, 5.2 Hz, 2H), 7.43-7.47 (m, 2H), 7.34 (d, J=8.8 Hz, 1H), 7.03-7.08 (m, 2H), 6.07 (q, J=7.2 Hz, 1H), 5.43 (s, 2H), 2.78-2.95 (m, 2H), 2.08 (d, J=6.8 Hz, 3H), 1.29-1.37 (m, 1H), 0.42-0.51 (m, 2H), 0.26-0.34 (m, 2H). SF NMR (376 MHz, CD₃OD) δ −78.22, −116.42. LC-MS: m/z 647.2 (M+H)⁺.

### 7-{4-[(5-chloro-2-pyridyl)methyl]-2,2-difluoro-3-oxo-3,4-dihydro-2H-1,4-benzoxazin-7-yl}-5-(cyclopropylmethyl)-3-[1-(5-fluoro-2-pyridyl)cyclopropyl]-1,3,4-triaza-3H-indene-6-carboxamide (Compound 263)

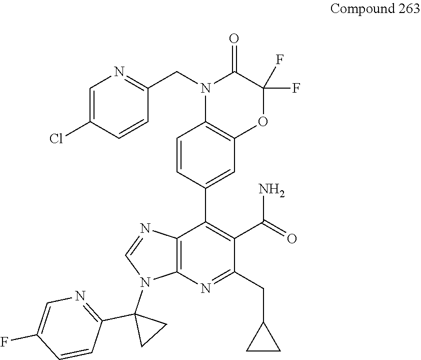 [SMILES: NC(=O)c1c(CC2CC2)nc2c(ncn2C2(c3ccc(F)cn3)CC2)c1-c1ccc2c(c1)OC(F)(F)C(=O)N2Cc1ccc(Cl)cn1]

Compound 263 was synthesized following the similar route of Example 51, using 1-(1-(5-fluoropyridin-2-yl)cyclopropyl)-1H-imidazol-5-amine and 4-((5-chloropyridin-2-yl)methyl)-2,2-difluoro-3-oxo-3,4-dihydro-2H-benzo[b][1,4]oxazine-7-carbaldehyde in step G. ¹H NMR (400 MHz, CD₃OD) δ 8.48-8.58 (m, 2H) 8.36 (d, J=2.8 Hz, 1H) 7.86 (dd, J=8.2, 2.4 Hz, 1H) 7.61 (d, J=2.0 Hz, 1H) 7.44-7.51 (m, 2H) 7.36-7.42 (m, 2H) 6.74 (dd, J=8.8, 3.6 Hz, 1H) 5.44 (s, 2H) 2.83 (d, J=7.2 Hz, 2H) 1.96-2.02 (m, 2H) 1.78-1.83 (m, 2H) 1.14-1.24 (m, 1H) 0.36-0.42 (m, 2H) 0.25 (m, J=4.8 Hz, 2H). ¹⁹F NMR (376 MHz, CD₃OD) δ −78.20 −132.88. LC-MS: m/z 660.3 (M+H)⁺.

### 7-{4-[(5-chloro-2-pyrimidinyl)methyl]-2,2-difluoro-3-oxo-3,4-dihydro-2H-1,4-benzoxazin-7-yl}-5-(cyclopropylmethyl)-3-[1-(5-fluoro-2-pyridyl)cyclopropyl]-1,3,4-triaza-3H-indene-6-carboxamide (Compound 264)

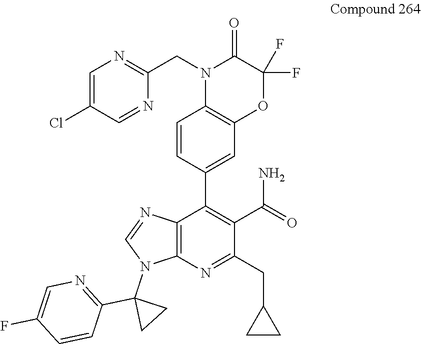 [SMILES: NC(=O)c1c(CC2CC2)nc2c(ncn2C2(c3ccc(F)cn3)CC2)c1-c1ccc2c(c1)OC(F)(F)C(=O)N2Cc1ncc(Cl)cn1]

Compound 264 was synthesized following the similar route of Example 51, using 1-(1-(5-fluoropyridin-2-yl)cyclopropyl)-1H-imidazol-5-amine in step G. ¹H NMR (400 MHz, CD₃OD) δ 8.80 (s, 2H), 8.54 (s, 1H), 8.36 (d, J=2.8 Hz, 1H), 7.62 (d, J=2.0 Hz, 1H), 7.49 (dd, J=8.4, 2.0 Hz, 1H), 7.39 (m, J=8.8, 2.8 Hz, 1H), 7.24 (d, J=8.6 Hz, 1H), 6.74 (dd, J=8.8, 4.0 Hz, 1H), 5.55 (s, 2H), 2.83 (d, J=6.8 Hz, 2H), 1.96-2.01 (m, 2H), 1.78-1.85 (m, 2H), 1.11-1.23 (m, 1H), 0.37-0.42 (m, 2H), 0.25 (q, J=4.8 Hz, 2H). ¹⁹F NMR (376 MHz, CD₃OD) δ −78.31, −132.88. LC-MS: m/z 661.1 (M+H)⁺.

### 7-{4-[(5-chloro-2-pyridyl)methyl]-2,2-difluoro-3-oxo-3,4-dihydro-2H-1,4-benzoxazin-7-yl}-5-(cyclopropylmethyl)-3-[1-(5-fluoro-2-pyridyl)ethyl]-1,3,4-triaza-3H-indene-6-carboxamide (Compound 265)

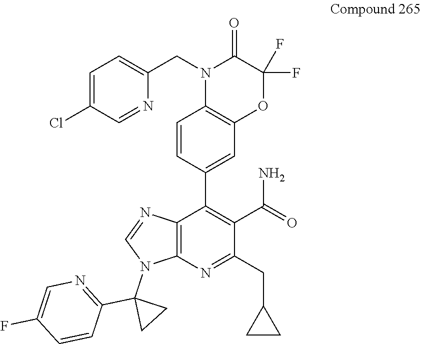 [SMILES: NC(=O)c1c(CC2CC2)nc2c(ncn2C2(c3ccc(F)cn3)CC2)c1-c1ccc2c(c1)OC(F)(F)C(=O)N2Cc1ccc(Cl)cn1]

Compound 265 was synthesized following the similar route of Example 51, using(S)-1-(1-(5-fluoropyridin-2-yl)ethyl)-1H-imidazol-5-amine and 4-((5-chloropyridin-2-yl)methyl)-2,2-difluoro-3-oxo-3,4-dihydro-2H-benzo[b][1,4]oxazine-7-carbaldehyde in step G. ¹H NMR (400 MHz, CD₃OD) δ 8.59 (s, 1H), 8.51 (d, J=1.6 Hz, 1H), 8.39 (s, 1H), 7.86 (dd, J=8.4, 2.4 Hz, 1H), 7.58 (d, J=1.6 Hz, 1H), 7.56 (dd, J=3.2, 1.6 Hz, 2H), 7.46-7.48 (m, 1H), 7.43-7.45 (m, 1H), 7.35 (d, J=8.8 Hz, 1H), 6.17 (d, J=7.2 Hz, 1H), 5.43 (s, 2H), 2.83 (dd, J=9.2, 7.2 Hz, 2H), 2.06 (d, J=7.2 Hz, 3H), 1.21-1.26 (m, 1H), 0.41-0.46 (m, 2H), 0.26 (d, J=4.4 Hz, 2H). ¹⁹F NMR (376 MHz, CD₃OD) δ −78.21, −130.28. LC-MS: m/z 648.2 (M+H)⁺.

### methyl 4-{1-[6-carbamoyl-5-(cyclopropylmethyl)-7-{2,2-difluoro-4-[(5-fluoro-2-pyrimidinyl)methyl]-3-oxo-3,4-dihydro-2H-1,4-benzoxazin-7-yl}-1,3,4-triaza-3H-inden-3-yl]ethyl}-1-piperidinecarboxylate (Compound 346)

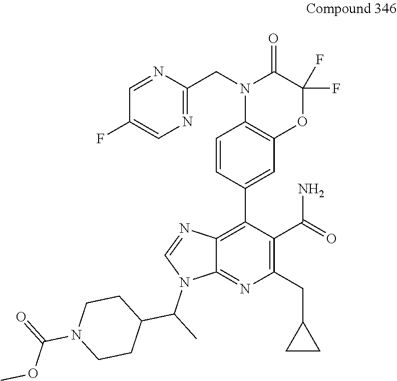 [SMILES: COC(=O)N1CCC(C(C)n2cnc3c(-c4ccc5c(c4)OC(F)(F)C(=O)N5Cc4ncc(F)cn4)c(C(N)=O)c(CC4CC4)nc32)CC1]

Compound 346 was synthesized following the similar route of Example 56, using methyl(S)-4-(1-(5-amino-1H-imidazol-1-yl)ethyl) piperidine-1-carboxylate and 2,2-difluoro-4-((5-fluoropyrimidin-2-yl)methyl)-3-oxo-3,4-dihydro-2H-benzo[b][1,4]oxazine-7-carbaldehyde in step A.

¹H NMR (400 MHz, DMSO-d₆) δ 8.93 (s, 2H), 8.47 (s, 1H), 7.84 (d, J=2.0 Hz, 1H), 7.67 (d, J=1.6 Hz, 1H), 7.52-7.55 (m, 2H), 7.38 (d, J=8.8 Hz, 1H), 5.52 (s, 2H), 4.53-4.57 (m, 1H), 3.99-4.04 (m, 1H), 3.85-3.91 (m, 1H), 3.55 (s, 3H), 2.71-2.83 (m, 3H), 2.21-2.29 (m, 1H), 1.82-1.88 (m, 1H), 1.60 (d, J=6.8 Hz, 3H), 1.25-1.30 (m, 1H), 0.98-1.16 (m, 4H), 0.42-0.46 (m, 2H), 0.28-0.31 (m, 2H). ¹⁹F NMR (376 MHz, DMSO-d₆) δ −75.04, −139.31. LC-MS: m/z 679.5 (M+H)⁺.

### methyl 4-[(S)-1-[6-carbamoyl-5-(cyclopropylmethyl)-7-{2,2-difluoro-4-[(5-fluoro-2-pyrimidinyl)methyl]-3-oxo-3,4-dihydro-2H-1,4-benzoxazin-7-yl}-1,3,4-triaza-3H-inden-3-yl]ethyl]-1-piperidinecarboxylate (Compound 347)

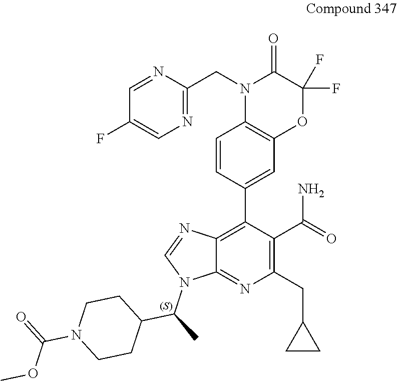

Compound 346 (13.4 mg) was purified by SFC (system: Waters SFC 150; Column name: DAICEL CHIRALPAK® AS; Column size: 250*30 mm 10 μm; Mobile Phase A: Supercritical CO₂; Mobile Phase B: MeOH (+0.1% 7.0 mol/l Ammonia in MEOH):ACN=80:20; Gradient: A/B=45/55; Flow rate: 145 mL/min; Column Temp: RT) to afford methyl 4-[(S)-1-[6-carbamoyl-5-(cyclopropylmethyl)-7-{2,2-difluoro-4-[(5-fluoro-2-pyrimidinyl)methyl]-3-oxo-3,4-dihydro-2H-1,4-benzoxazin-7-yl}-1,3,4-triaza-3H-inden-3-yl]ethyl]-1-piperidinecarboxylate (5.16 mg, 38.51%).

Rt=5.51 min. ¹H NMR (400 MHz, DMSO-d₆) δ 8.93 (s, 2H), 8.47 (s, 1H), 7.84 (s, 1H), 7.67 (d, J=2.0 Hz, 1H), 7.52-7.55 (m, 2H), 7.38 (d, J=8.8 Hz, 1H), 5.52 (s, 2H), 4.51-4.59 (m, 1H), 3.97-4.05 (m, 1H), 3.84-3.91 (m, 1H), 3.55 (s, 3H), 2.71-2.83 (m, 3H), 2.55-2.65 (m, 1H), 2.19-2.30 (m, 1H), 1.81-1.88 (m, 1H), 1.60 (d, J=6.8 Hz, 3H), 0.83-1.25 (m, 4H), 0.43-0.46 (m, 2H), 0.29-0.31 (m, 2H). ¹⁹F NMR (376 MHz, DMSO-d₆) δ −75.04, −139.31. LC-MS: m/z 679.5 (M+H)⁺.

### 7-{4-[(5-chloro-2-pyridyl)methyl]-2,2-difluoro-3-oxo-3,4-dihydro-2H-1,4-benzoxazin-7-yl}-5-(cyclopropylmethyl)-3-[(S)-1-(5-fluoro-2-pyridyl)ethyl]-1,3,4-triaza-3H-indene-6-carboxamide and 7-{4-[(5-chloro-2-pyridyl)methyl]-2,2-difluoro-3-oxo-3,4-dihydro-2H-1,4-benzoxazin-7-yl}-5-(cyclopropylmethyl)-3-[(R)-1-(5-fluoro-2-pyridyl)ethyl]-1,3,4-triaza-3H-indene-6-carboxamide (Compound 266 and Compound 267)

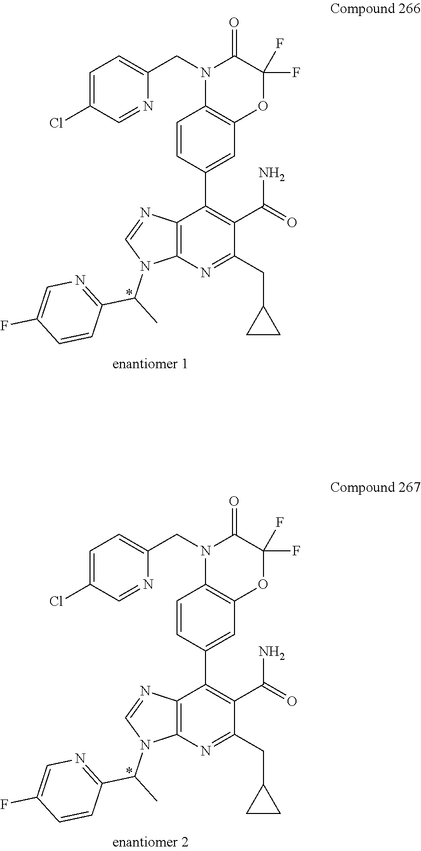 [SMILES: CC(c1ccc(F)cn1)n1cnc2c(-c3ccc4c(c3)OC(F)(F)C(=O)N4Cc3ccc(Cl)cn3)c(C(N)=O)c(CC3CC3)nc21 ; CC(c1ccc(F)cn1)n1cnc2c(-c3ccc4c(c3)OC(F)(F)C(=O)N4Cc3ccc(Cl)cn3)c(C(N)=O)c(CC3CC3)nc21]

7-{4-[(5-chloro-2-pyridyl)methyl]-2,2-difluoro-3-oxo-3,4-dihydro-2H-1,4-benzoxazin-7-yl}-5-(cyclopropylmethyl)-3-[1-(5-fluoro-2-pyridyl)ethyl]-1,3,4-triaza-3H-indene-6-carboxamide (270 mg) was purified by SFC (system: Waters SFC 21; Column name: DAICEL CHIRALCEL OD; Column size: (250 mm*30 mm, 10 μm); mobile phase A: Supercritical CO₂; mobile phase B: i-PrOH (+0.1% NH₃·H₂O)]; Gradient: A/B=60/40; Flow rate: 80 mL/min; Column Temp: RT) to afford 7-{4-[(5-chloro-2-pyridyl)methyl]-2,2-difluoro-3-oxo-3,4-dihydro-2H-1,4-benzoxazin-7-yl}-5-(cyclopropylmethyl)-3-[(S)-1-(5-fluoro-2-pyridyl)ethyl]-1,3,4-triaza-3H-indene-6-carboxamide (67.38 mg) and 7-{4-[(5-chloro-2-pyridyl)methyl]-2,2-difluoro-3-oxo-3,4-dihydro-2H-1,4-benzoxazin-7-yl}-5-(cyclopropylmethyl)-3-[(R)-1-(5-fluoro-2-pyridyl)ethyl]-1,3,4-triaza-3H-indene-6-carboxamide (7.48 mg).

Compound 266, enantiomer 1: Rt=1.35 min. ¹H NMR (400 MHz, CD₃OD) δ 8.59 (s, 1H), 8.51 (d, J=2.4 Hz, 1H), 8.39 (s, 1H), 7.86 (dd, J=8.4, 2.4 Hz, 1H), 7.54-7.60 (m, 3H), 7.42-7.47 (m, 2H), 7.31-7.38 (m, 1H), 6.17 (q, J=7.2 Hz, 1H), 5.43 (s, 2H), 2.78-2.87 (m, 2H), 2.06 (d, J=7.2 Hz, 3H), 1.18-1.26 (m, 1H), 0.40-0.47 (m, 2H), 0.26 (d, J=4.0 Hz, 2H). ¹⁹F NMR (376 MHz, CD₃OD) δ −78.21, −130.28. LC-MS: m/z 648.0 (M+H)⁺.

Compound 267, enantiomer 2: Rt=1.51 min. ¹H NMR (400 MHz, CD₃OD) δ 8.59 (s, 1H), 8.50 (d, J=2.0 Hz, 1H), 8.39 (s, 1H), 7.85 (dd, J=8.4, 2.4 Hz, 1H), 7.54-7.60 (m, 3H), 7.43-7.48 (m, 2H), 7.35 (d, J=8.4 Hz, 1H), 6.17 (q, J=7.2 Hz, 1H), 5.43 (s, 2H), 2.78-2.88 (m, 2H), 2.06 (d. J=7.2 Hz, 3H), 1.17-1.26 (m, 1H), 0.39-0.47 (m, 2H), 0.25 (dd, J=3.2, 1.6 Hz, 2H). ¹⁹F NMR (376 MHz, CD₃OD) δ −78.18, −130.23. LC-MS: m/z 648.2 (M+H)⁺.

### 7-{4-[(5-chloro-2-pyridyl)methyl]-2,2-difluoro-3-oxo-3,4-dihydro-2H-1,4-benzoxazin-7-yl}-5-(cyclopropylmethyl)-3-[1-(5-fluoro-2-pyrimidinyl)ethyl]-1,3,4-triaza-3H-indene-6-carboxamide (Compound 268)

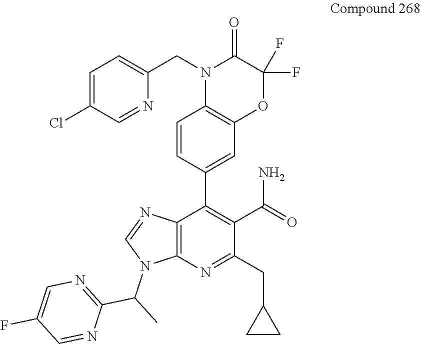 [SMILES: CC(c1ncc(F)cn1)n1cnc2c(-c3ccc4c(c3)OC(F)(F)C(=O)N4Cc3ccc(Cl)cn3)c(C(N)=O)c(CC3CC3)nc21]

Compound 268 was synthesized following the similar route of Example 51, using 1-(1-(5-fluoropyrimidin-2-yl)ethyl)-1H-imidazol-5-amine and 4-((5-chloropyridin-2-yl)methyl)-2,2-difluoro-3-oxo-3,4-dihydro-2H-benzo[b][1,4]oxazine-7-carbaldehyde in step G.

¹H NMR (400 MHz, CD₃OD) δ 8.67 (s, 2H), 8.62 (s, 1H), 8.51 (d, J=2.0 Hz, 1H), 7.86 (dd, J=2.4, 8.4 Hz, 1H), 7.58 (d, J=1.6 Hz, 1H), 7.50-7.44 (m, 2H), 7.36 (d, J=8.4 Hz, 1H), 6.36-6.19 (m, 1H), 5.44 (s, 2H), 2.78 (t, J=7.2 Hz, 2H), 2.10 (d, J=7.2 Hz, 3H), 1.20-1.13 (m, 1H), 0.42-0.34 (m, 2H), 0.23-0.16 (m, 2H). ¹⁹F NMR (376 MHz, CD₃OD) δ −78.19, −141.34. LC-MS: m/z 649.0 (M+H)⁺.

### 5-(cyclopropylmethyl)-7-{4-[(5-fluoro-2-pyridyl)methyl]-2,2-dimethyl-3-oxo-3,4-dihydro-2H-1,4-benzoxazin-7-yl}-3-(2,2,2-trifluoroethyl)-1,3,4-triaza-3H-indene-6-carboxamide (Compound 269)

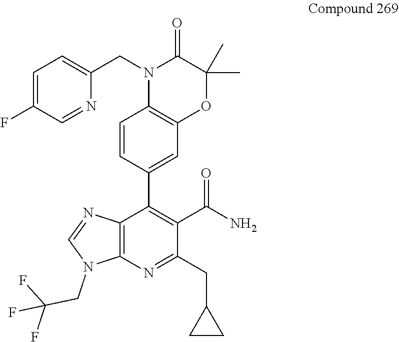 [SMILES: CC1(C)Oc2cc(-c3c(C(N)=O)c(CC4CC4)nc4c3ncn4CC(F)(F)F)ccc2N(Cc2ccc(F)cn2)C1=O]

Compound 269 was synthesized following the similar route of Example 50, using 1-(2,2,2-trifluoroethyl)-1H-imidazol-5-amine and 4-((5-fluoropyridin-2-yl)methyl)-2,2-dimethyl-3-oxo-3,4-dihydro-2H-benzo[b][1,4]oxazine-7-carbaldehyde in step A.

¹H NMR (400 MHz, CD₃OD) δ 8.44-8.38 (m, 2H), 7.63-7.56 (m, 1H), 7.40-7.36 (m, 1H), 7.35-7.30 (m, 1H), 7.29-7.24 (m, 1H), 7.15-7.09 (m, 1H), 5.30 (s, 2H), 5.22-5.15 (m, 2H), 2.90 (d, J=6.8 Hz, 2H), 1.58 (s, 6H), 1.34-1.29 (m, 1H), 0.55-0.45 (m, 2H), 0.40-0.29 (m, 2H). ¹⁹F NMR (376 MHz, CD₃OD) δ −73.22, −130.96. LC-MS: m/z 583.2 (M+H)⁺.

### 7-{4-[(5-chloro-2-pyridyl)methyl]-2,2-dimethyl-3-oxo-3,4-dihydro-2H-1,4-benzoxazin-7-yl}-5-(cyclopropylmethyl)-3-[(S)-1-methyl-2-trifluoromethoxyethyl]-1,3,4-triaza-3H-indene-6-carboxamide (Compound 270)

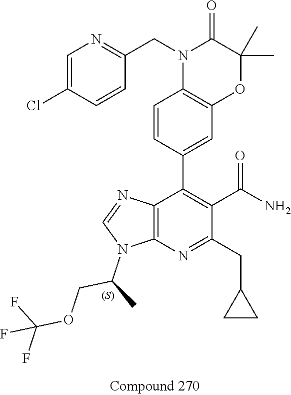

Compound 270 was synthesized following the similar route of Example 51, using(S)-1-(1-(trifluoromethoxy)propan-2-yl)-1H-imidazol-5-amine and 4-[(5-chloropyridin-2-yl)methyl]-2,2-dimethyl-3-oxo-3,4-dihydro-2H-benzo[b][1,4]oxazine-7-carbaldehyde in step G.

¹H NMR (400 MHz, DMSO-d₆) δ δ 8.59 (d, J=2.4 Hz, 1H), 8.49 (s, 1H), 7.94 (dd, J₁=2.4 Hz, J₂=8.4 Hz, 1H), 7.75 (d, J=2.0 Hz, 1H), 7.48 (d, J=0.8 Hz, 1H), 7.37-7.40 (m, 2H), 7.33 (dd, J₁=2.0 Hz, J₂=8.4 Hz, 1H), 7.07 (d, J=8.4 Hz, 1H), 5.25 (s, 2H), 5.07-5.12 (m, 1H), 4.76-4.81 (m, 1H), 4.55 (dd, J₁=4.8 Hz, J₂=10.4 Hz, 1H), 2.75-2.77 (m, 2H), 1.63 (d, J=7.2 Hz, 3H), 1.51 (s, 6H), 1.30 (m, 1H), 0.40-0.45 (m, 2H), 0.26-0.30 (m, 2H). ¹⁹F NMR (376 MHz, DMSO-d₆) δ −59.00. LC-MS: m/z 643.3 (M+H)⁺.

### 7-{4-[(5-chloro-2-pyrimidinyl)methyl]-2,2-difluoro-3-oxo-3,4-dihydro-2H-1,4-benzoxazin-7-yl}-5-(cyclopropylmethyl)-3-[1-(methoxymethyl)cyclopropyl]-1,3,4-triaza-3H-indene-6-carboxamide (Compound 271)

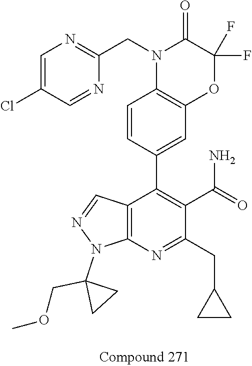 [SMILES: COCC1(n2ncc3c(-c4ccc5c(c4)OC(F)(F)C(=O)N5Cc4ncc(Cl)cn4)c(C(N)=O)c(CC4CC4)nc32)CC1]

Compound 271 was synthesized following the similar route of Example 51, using 1-(1-(methoxymethyl)cyclopropyl)-1H-imidazol-5-amine in step G.

¹H NMR (400 MHz, CD₃OD) δ 8.79 (s, 2H), 8.32 (s, 1H), 7.57 (d, J=1.6 Hz, 1H), 7.44 (dd, J=1.6, 8.4 Hz, 1H), 7.22 (d, J=8.4 Hz, 1H), 5.54 (s, 2H), 3.71 (s, 2H), 3.29 (s, 3H), 2.91 (d, J=7.2 Hz, 2H), 1.39-1.36 (m, 2H), 1.35-1.31 (m, 1H), 1.29-1.26 (m, 2H), 0.54-0.48 (m, 2H), 0.39-0.34 (m, 2H). ¹⁹F NMR (376 MHz, CD₃OD) δ −78.31. LC-MS: m/z 610.1 (M+H)⁺.

### 7-{4-[(5-chloro-2-pyrimidinyl)methyl]-2,2-difluoro-3-oxo-3,4-dihydro-2H-1,4-benzoxazin-7-yl}-5-(cyclopropylmethyl)-3-[1-(methoxymethyl)cyclopropyl]-1,3,4-triaza-3H-indene-6-carboxamide (Compound 272)

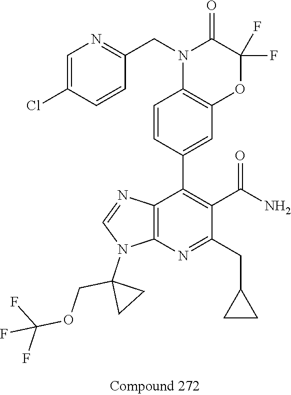 [SMILES: NC(=O)c1c(CC2CC2)nc2c(ncn2C2(COC(F)(F)F)CC2)c1-c1ccc2c(c1)OC(F)(F)C(=O)N2Cc1ccc(Cl)cn1]

Compound 272 was synthesized following the similar route of Example 51, using 1-(1-((trifluoromethoxy)methyl)cyclopropyl)-1H-imidazol-5-amine and 4-((5-chloropyridin-2-yl)methyl)-2,2-difluoro-3-oxo-3,4-dihydro-2H-benzo[b][1,4]oxazine-7-carbaldehyde in step G. ¹H NMR (400 MHz, DMSO-d₆) δ 8.58 (d, J=2.4 Hz, 1H), 8.43 (s, 1H), 7.99 (dd, J₁=2.4 Hz, J₂=8.4 Hz, 1H), 7.83 (s, 1H), 7.63 (d, J=1.6 Hz, 1H), 7.59 (d, J=8.4 Hz, 1H), 7.55 (s, 1H), 7.52 (dd, J₁=2.0 Hz, J₂=8.4 Hz, 1H), 7.39 (d, J=8.4 Hz, 1H), 5.43 (s, 2H), 4.51 (s, 2H), 2.79 (d, J=6.8 Hz, 2H), 1.41-1.44 (m, 2H), 1.35-1.39 (m, 2H), 1.22-1.27 (m, 1H), 0.41-0.45 (m, 2H), 0.29-0.33 (m, 2H). ¹⁹F NMR (376 MHz, DMSO-d₆) δ −58.45, −74.60. LC-MS: m/z 663.1 (M+H)⁺.

### 7-{4-[(5-chloro-2-pyridyl)methyl]-2,2-difluoro-3-oxo-3,4-dihydro-2H-1,4-benzoxazin-7-yl}-5-(cyclopropylmethyl)-3-[1-(difluoromethoxymethyl)cyclopropyl]-1,3,4-triaza-3H-indene-6-carboxamide (Compound 273)

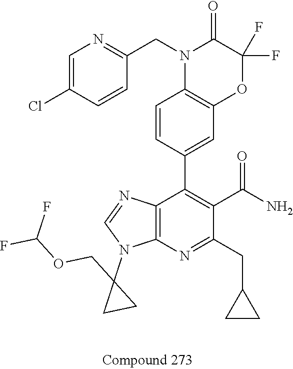 [SMILES: NC(=O)c1c(CC2CC2)nc2c(ncn2C2(COC(F)F)CC2)c1-c1ccc2c(c1)OC(F)(F)C(=O)N2Cc1ccc(Cl)cn1]

Compound 273 was synthesized following the similar route of Example 51, using 1-(1-((difluoromethoxy)methyl)cyclopropyl)-1H-imidazol-5-amine and 4-((5-chloropyridin-2-yl)methyl)-2,2-difluoro-3-oxo-3,4-dihydro-2H-benzo[b][1,4]oxazine-7-carbaldehyde in step G. ¹H NMR (400 MHz, DMSO-d₆) δ 8.58 (d, J=2.4 Hz, 1H), 8.37 (s, 1H), 7.99 (dd, J₁=2.4 Hz, J₂=8.4 Hz, 1H), 7.80 (s, 1H), 7.62 (d, J=1.6 Hz, 1H), 7.59 (d, J=8.8 Hz, 1H), 7.54 (s, 1H), 7.51 (dd, J₁=1.6 Hz, J₂=8.8 Hz, 1H), 7.39 (d, J=8.8 Hz, 1H), 6.64 (t, J=75.4 Hz, 1H), 5.43 (s, 2H), 4.22 (s, 2H), 2.79 (d, J=6.8 Hz, 2H), 1.35-1.39 (m, 2H), 1.29-1.32 (m, 2H), 1.23-1.27 (m, 1H), 0.41-0.46 (m, 2H), 0.29-0.33 (m, 2H). ¹⁹F NMR (376 MHz, DMSO-d₆) δ −74.59, −82.39. LC-MS: m/z 645.3 (M+H)⁺.

### 7-{4-[(5-chloro-2-pyrimidinyl)methyl]-2,2-difluoro-3-oxo-3,4-dihydro-2H-1,4-benzoxazin-7-yl}-5-(cyclopropylmethyl)-3-[1-(trifluoromethyl)cyclopropyl]-1,3,4-triaza-3H-indene-6-carboxamide (Compound 274)

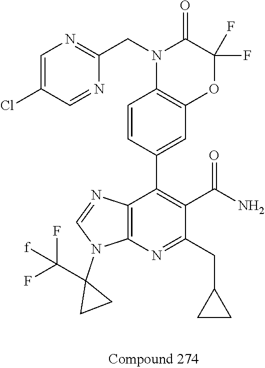

Compound 274 was synthesized following the similar route of Example 51, using 1-(1-(trifluoromethyl)cyclopropyl)-1H-imidazol-5-amine in step G. ¹H NMR (400 MHz, DMSO-d₆) δ 8.97 (s, 2H), 8.63 (s, 1H), 7.84 (s, 1H), 7.63 (d, J=2 Hz, 1H), 7.59 (s, 1H), 7.51 (dd, J₁=2 Hz, J₂=8.8 Hz, 1H), 7.41 (d, J=8.8 Hz, 1H), 5.51 (s, 2H), 2.79 (d, J=7.2 Hz, 2H), 1.75-1.79 (m, 2H), 1.67-1.69 (m, 2H), 1.23-1.28 (m, 1H), 0.41-0.43 (m, 2H), 0.31-0.33 (m, 2H). ¹⁹F NMR (376 MHz, DMSO-d₆) δ −71.83, −74.96. LC-MS: m/z 634.3 (M+H)⁺.

### 7-{4-[(5-chloro-2-pyrimidinyl)methyl]-2,2-difluoro-3-oxo-3,4-dihydro-2H-1,4-benzoxazin-7-yl}-5-(cyclopropylmethyl)-3-[1-(difluoromethyl)cyclopropyl]-1,3,4-triaza-3H-indene-6-carboxamide (Compound 275)

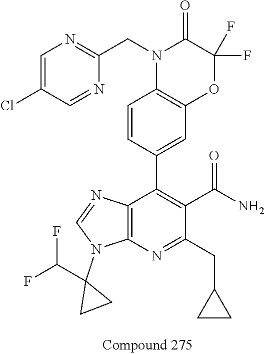 [SMILES: NC(=O)c1c(CC2CC2)nc2c(ncn2C2(C(F)F)CC2)c1-c1ccc2c(c1)OC(F)(F)C(=O)N2Cc1ncc(Cl)cn1]

Compound 275 was synthesized following the similar route of Example 51, using 1-(1-(difluoromethyl)cyclopropyl)-1H-imidazol-5-amine in step G. ¹H NMR (400 MHz, DMSO-d₆) δ 8.97 (s, 2H), 8.46 (s, 1H), 7.82 (d, J=1.6 Hz, 1H), 7.64 (d, J=1.6 Hz, 1H), 7.56 (d, J=2.0 Hz, 1H), 7.51 (dd, J₁=1.6 Hz, J₂=8.4 Hz, 1H), 7.40 (d, J=8.8 Hz, 1H), 6.12 (t, J=54.6 Hz, 1H), 5.51 (s, 2H), 2.66 (d, J=2.0 Hz, 2H), 1.53 (s, 4H), 1.23-1.26 (m, 1H), 0.40-0.44 (m, 2H), 0.29-0.33 (m, 2H). ¹⁹F NMR (376 MHz, DMSO-d₆) δ −74.95, −118.93. LC-MS: m/z 616.8 (M+H)⁺.

### 7-{4-[(5-chloro-2-pyrimidinyl)methyl]-2,2-difluoro-3-oxo-3,4-dihydro-2H-1,4-benzoxazin-7-yl}-5-(cyclopropylmethyl)-2-methyl-3-[1-(trifluoromethyl)cyclopropyl]-1,3,4-triaza-3H-indene-6-carboxamide (compound 276)

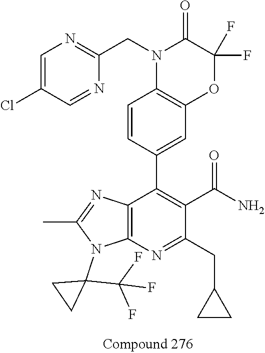 [SMILES: Cc1nc2c(-c3ccc4c(c3)OC(F)(F)C(=O)N4Cc3ncc(Cl)cn3)c(C(N)=O)c(CC3CC3)nc2n1C1(C(F)(F)F)CC1]

Compound 276 was synthesized following the similar route of Example 51, using 2-methyl-1-(1-(trifluoromethyl)cyclopropyl)-1H-imidazol-5-amine in step G. ¹H NMR (400 MHz, DMSO-d₆) δ 8.97 (s, 2H), 7.82 (s, 1H), 7.59 (d, J=1.2 Hz, 1H), 7.55 (s, 1H), 7.46 (dd, J₁=1.6 Hz, J₂=8.4 Hz, 1H), 7.39 (d, J=8.8 Hz, 1H), 5.51 (s, 2H), 2.76 (d, J=6.8 Hz, 2H), 2.60 (s, 3H), 1.74-1.77 (m, 4H), 1.23 (m, 1H), 0.41-0.43 (m, 2H), 0.31-0.32 (m, 2H). LC-MS: m/z 648.4 (M+H)⁺.

### 7-{4-[(5-chloro-2-pyrimidinyl)methyl]-2,2-difluoro-3-oxo-3,4-dihydro-2H-1,4-benzoxazin-7-yl}-5-(cyclopropylmethyl)-2-isopropyl-3-[1-(trifluoromethyl)cyclopropyl]-1,3,4-triaza-3H-indene-6-carboxamide (Compound 345)

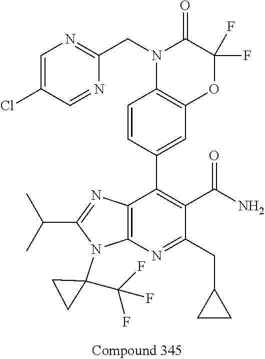 [SMILES: CC(C)c1nc2c(-c3ccc4c(c3)OC(F)(F)C(=O)N4Cc3ncc(Cl)cn3)c(C(N)=O)c(CC3CC3)nc2n1C1(C(F)(F)F)CC1]

Compound 345 was synthesized following the similar route of Example 51, using 2-isopropyl-1-(1-(trifluoromethyl)cyclopropyl)-1H-imidazol-5-amine in step A.

¹H NMR (400 MHz, CDCl₃) δ 8.69 (s, 2H), 7.75 (d, J=2.0 Hz, 1H), 7.56 (dd, J=2.0, 8.4 Hz, 1H), 6.99 (d, J=8.8 Hz, 1H), 5.57 (br s, 1H), 5.45 (s, 2H), 5.36 (br s, 1H), 3.48 (q, J=6.4 Hz, 1H), 2.90 (d, J=6.4 Hz, 2H), 1.84 (m, 1H), 1.80-1.75 (m, 1H), 1.70 (m, 1H), 1.44 (d, J=6.8 Hz, 3H), 1.40-1.33 (m, 5H), 0.49 (br d, J=8.0 Hz, 2H), 0.35 (br s, 2H). ¹⁹F NMR (376 MHz, CDCl₃) δ −72.71, −76.35. LC-MS: m/z 676.2 (M+H)⁺.

### Example 58

### 5-(cyclopropylmethyl)-7-{2,2-difluoro-4-[(5-fluoro-2-pyrimidinyl)methyl]-3-oxo-3,4-dihydro-2H-1,4-benzoxazin-7-yl}-3-[(S)-1-(p-fluorophenyl)ethyl]-1,2,3,4-tetraaza-3H-indene-6-carboxamide (compound 278)

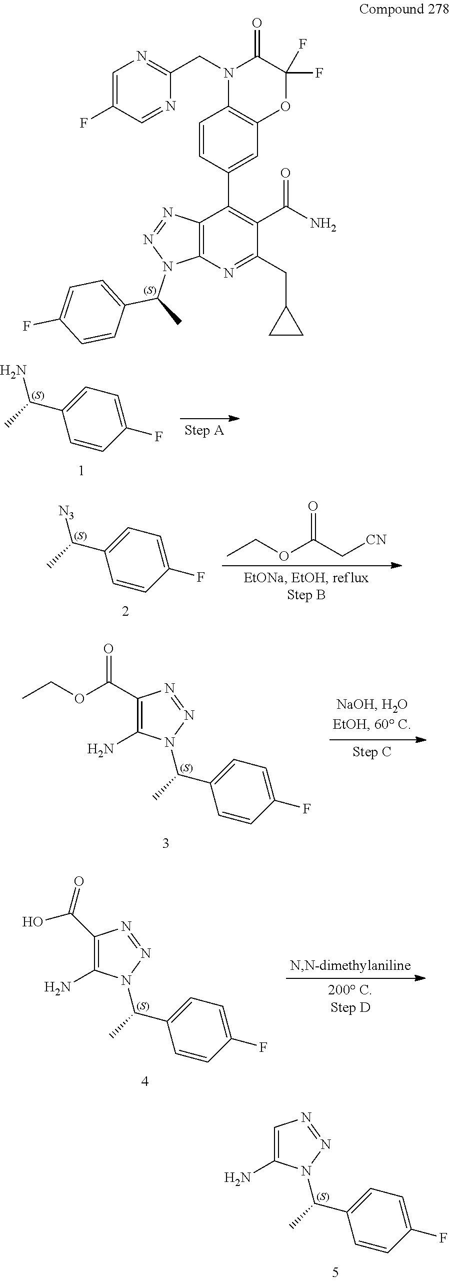

### Step A: 1-[(1S)-1-azidoethyl]-4-fluoro-benzene

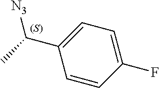

To a solution of (1S)-1-(4-fluorophenyl) ethanamine (1 g, 7.19 mmol), DMAP (1.05 g, 8.62 mmol) in CH₃CN (8 mL) was added 2-azido-1,3-dimethyl-4,5-dihydroimidazol-1-ium;hexafluorophosphate (2.46 g, 8.62 mmol) in CH₃CN (4 mL). The mixture was stirred at 30° C. for 1 hr. The mixture was concentrated to give a residue. The residue was purified by flash silica gel chromatography (PE:EA=1:0 to 10:1 to afford 1-[(1S)-1-azidoethyl]-4-fluoro-benzene (1.33 g, 90.53%) as a colorless oil. LC-MS: m/z 123.1 (M-N₃)⁺.

### Step B: ethyl 5-amino-1-[(1S)-1-(4-fluorophenyl)ethyl]triazole-4-carboxylate

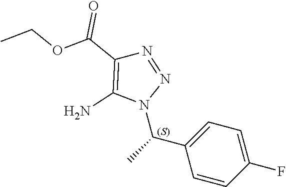

To a solution of 1-[(1S)-1-azidoethyl]-4-fluoro-benzene (1.33 g, 8.05 mmol) and ethyl 2-cyanoacetate (1.37 g, 12.08 mmol, 1.29 mL) in EtOH (5 mL) was added NaOEt (6.30 g, 18.52 mmol). The mixture was stirred at 80° C. for 4 hr. The mixture was concentrated to give a residue. The residue was purified by flash silica gel chromatography (PE:EA=1:0 to 0:1 to afford ethyl 5-amino-1-[(1S)-1-(4-fluorophenyl)ethyl]triazole-4-carboxylate (527 mg, 23.52) as a pale yellow solid. LC-MS: m/z 279.1 (M+H)+.

### Step C: 5-amino-1-[(1S)-1-(4-fluorophenyl)ethyl]triazole-4-carboxylic acid

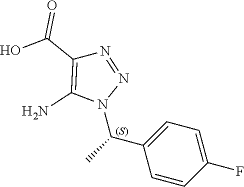

To a solution of ethyl 5-amino-1-[(1S)-1-(4-fluorophenyl)ethyl]triazole-4-carboxylate (527 mg, 1.89 mmol) in EtOH (6 mL) and H₂O (4 mL) was added NaOH (227.24 mg, 5.68 mmol). The mixture was stirred at 60° C. for 2 hr. The mixture was concentrated to remove the solvent, then diluted with H₂O (10 mL), adjusted to pH 5~6 with HCl (2N), extracted with DCM (30 mL×3), the combined organic layers were washed with brine (100 mL), dried over Na₂SO₄, filtered and concentrated to afford 5-amino-1-[(1S)-1-(4-fluorophenyl)ethyl]triazole-4-carboxylic acid (439 mg, 92.64%) as a pale yellow solid, which was used into the next step without further purification. ¹H NMR (400 MHz, DMSO-d₆) δ 7.24-7.39 (m, 2H), 7.07-7.23 (m, 2H), 6.23 (br s, 2H), 5.65 (q, J=6.8 Hz, 1H), 1.82 (d, J=6.8 Hz, 3H). LC-MS: m/z 251.0 (M+H)⁺.

### Step D: 3-[(1S)-1-(4-fluorophenyl)ethyl]triazol-4-amine

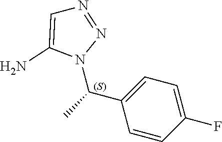

To a solution of 5-amino-1-[(1S)-1-(4-fluorophenyl)ethyl]triazole-4-carboxylic acid (439 mg, 1.75 mmol) in NMP (30 mL) was stirred at 220° C. for 25 min (Flow Chem). The mixture was diluted with H₂O (100 mL), extracted with EA (50 mL×3), the combined organic layers were washed with brine (60 mL×3), dried over Na₂SO₄, filtered and concentrated to give a residue. The residue was purified by prep-HPLC (column: Welch Xtimate C18 150×30 mm×5 μm; mobile phase: [water (NH₄HCO₃)-ACN]; gradient: 28%-58% B over 7 min) to afford 3-[(1S)-1-(4-fluorophenyl)ethyl]triazol-4-amine (45 mg, 12.44%) as a pale yellow solid. ¹H NMR (400 MHz, METHANOL-d₄) δ 7.28 (dd, J=8.8, 5.6 Hz, 2H), 7.06 (t, J=8.8 Hz, 2H), 6.95 (s, 1H), 5.59 (q, J=7.2 Hz, 1H), 1.92 (d, J=7.2 Hz, 3H). LC-MS: m/z 207.0 (M+H)⁺.

### 5-(cyclopropylmethyl)-7-{2,2-difluoro-4-[(5-fluoro-2-pyrimidinyl)methyl]-3-oxo-3,4-dihydro-2H-1,4-benzoxazin-7-yl}-3-[(S)-1-(p-fluorophenyl)ethyl]-1,2,3,4-tetraaza-3H-indene-6-carboxamide (Compound 278)

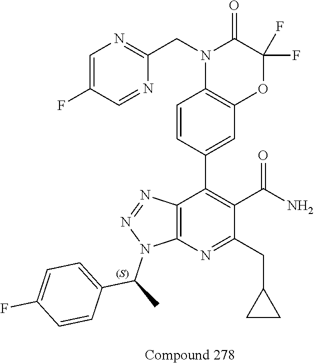

Compound 278 was then synthesized following the similar route of Example 56, using 3-[(1S)-1-(4-fluorophenyl)ethyl]triazol-4-amine in step G. ¹H NMR (400 MHz, CD₃OD) δ 8.72 (s, 1H), 8.66 (d, J=8.4 Hz, 1H), 7.72 (d, J=2.0 Hz, 1H), 7.55-7.59 (m, 2H), 7.28 (d, J=8.4 Hz, 1H), 7.01-7.10 (m, 3H), 6.38-6.46 (m, 1H), 5.55 (s, 2H), 2.89-2.99 (m, 2H), 2.20 (d, J=7.2 Hz, 3H), 0.88-0.93 (m, 1H), 0.50-0.55 (m, 2H), 0.29-0.35 (m, 2H). ¹⁹F NMR (376 MHz, CD₃OD) δ −78.42, −116.08, −141.11. LC-MS: m/z 633.3 (M+H)⁺.

### 7-{4-[(5-chloro-2-pyrimidinyl)methyl]-2,2-dimethyl-3-oxo-3,4-dihydro-2H-1,4-benzoxazin-7-yl}-5-isobutyl-3-isopropyl-1-oxa-2,4-diaza-6-indenecarboxamide (compound 279)

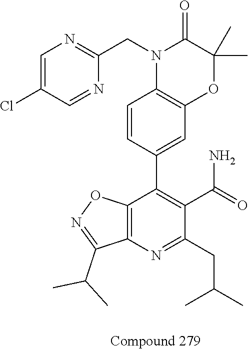 [SMILES: CC(C)Cc1nc2c(C(C)C)noc2c(-c2ccc3c(c2)OC(C)(C)C(=O)N3Cc2ncc(Cl)cn2)c1C(N)=O]

Compound 279 was synthesized following the similar route of Example 50, using 3-isopropylisoxazol-4-amine, 5-methyl-3-oxohexanamide and 4-((5-chloropyrimidin-2-yl)methyl)-2,2-dimethyl-3-oxo-3,4-dihydro-2H-benzo[b][1,4]oxazine-7-carbaldehyde in step A.

¹H NMR (400 MHz, DMSO-d₆) δ 8.93 (s, 2H), 7.93 (d, J=1.6 Hz, 1H), 7.77 (d, J=1.6 Hz, 1H), 7.34 (d, J=2.0 Hz, 1H), 7.28 (dd, J₁=2.0 Hz, J₂=8.4 Hz, 1H), 7.12 (d, J=8.4 Hz, 1H), 5.35 (s, 2H), 3.45-3.52 (m, 1H), 2.80 (d, J=7.2 Hz, 2H), 2.28-2.33 (m, 1H), 1.50 (s, 6H), 1.48 (d, J=7.2 Hz, 6H), 0.94 (d, J=6.4 Hz, 6H). LC-MS: m/z 563.4 (M+H)⁺.

### 7-{4-[(5-chloro-2-pyrimidinyl)methyl]-2,2-dimethyl-3-oxo-3,4-dihydro-2H-1,4-benzoxazin-7-yl}-5-isobutyl-3-isopropyl-1-oxa-2,4-diaza-6-indenecarboxamide (Compound 280)

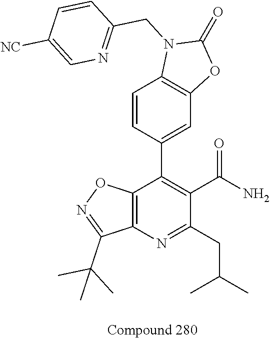 [SMILES: CC(C)Cc1nc2c(C(C)(C)C)noc2c(-c2ccc3c(c2)oc(=O)n3Cc2ccc(C#N)cn2)c1C(N)=O]

Compound 280 was synthesized following the similar route of Example 50, using 3-(tert-butyl) isoxazol-4-amine, 5-methyl-3-oxohexanamide and 6-((6-formyl-2-oxobenzo[d]oxazol-3(2H)-yl)methyl) nicotinonitrile in step A. ¹H NMR (400 MHz, DMSO-d₆) δ 8.96 (s, 1H), 8.31 (d, J=7.6 Hz, 1H), 7.93 (s, 1H), 7.75 (d, J=1.6 Hz, 1H), 7.59 (d, J=7.6 Hz, 1H), 7.34 (d, J=1.6 Hz, 1H), 7.28 (dd, J₁=1.2 Hz, J₂=7.6 Hz, 1H), 7.13 (d, J=8.0 Hz, 1H), 5.36 (s, 2H), 2.81 (d, J=7.2 Hz, 2H), 1.98-2.00 (m, 1H), 1.55 (s, 9H), 1.52 (s, 6H), 0.95 (d, J=6.4 Hz, 6H). LC-MS: m/z 567.5 (M+H)⁺.

### (S)-3-[(p-fluorophenyl)methyl]-7-{4-[(5-fluoro-2-pyridyl)methyl]-3-oxo-3,4-dihydrospiro[1,4-benzoxazine-2,1′-cyclopropan]-7-yl}-5-isobutyl-3-methyl-1-oxo-2,4-diaza-6-indancarboxamide (Compound 281)

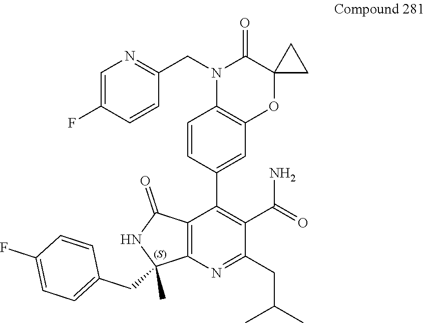

Compound 281 was synthesized following the similar route of Example 50, using(S)-4-amino-5-(4-fluorobenzyl)-5-methyl-1,5-dihydro-2H-pyrrol-2-one, 5-methyl-3-oxohexanamide and 4-((5-fluoropyridin-2-yl)methyl)-3-oxo-3,4-dihydrospiro[benzo[b][1,4]oxazine-2, l′-cyclopropane]-7-carbaldehyde in step A.

¹H NMR (400 MHz, DMSO-d₆) δ 8.67 (s, 1H), 8.53 (d, J=3.2 Hz, 1H), 7.70-7.74 (m, 2H), 7.42-7.47 (m, 2H), 6.97 (d, J=8.4 Hz, 1H), 6.89 (t, J=9 Hz, 2H), 6.81-6.84 (m, 2H), 6.72-6.74 (m, 2H), 5.22 (s, 2H), 3.22 (d, J=13.2 Hz, 1H), 3.03 (d, J=13.2 Hz, 1H), 2.69-2.80 (m, 2H), 2.32-2.39 (m, 1H), 1.53 (s, 3H), 1.30-1.37 (m, 2H), 1.21-1.27 (m, 2H), 1.00 (d, J=6.8 Hz, 3H), 0.90 (d, J=6.4 Hz, 3H). ¹⁹F NMR (376 MHz, DMSO-d₆) δ −116.69, −129.46. LC-MS: m/z 638.5 (M+H)⁺.

### (S)-3-[(p-fluorophenyl)methyl]-7-{3-[(5-fluoro-2-pyridyl)methyl]-2-oxo-2,3-dihydro-1,3-benzoxazolidin-6-yl}-3-methyl-1-oxo-5-(2,2,2-trifluoroethyl)-2,4-diaza-6-indancarboxamide (Compound 282)

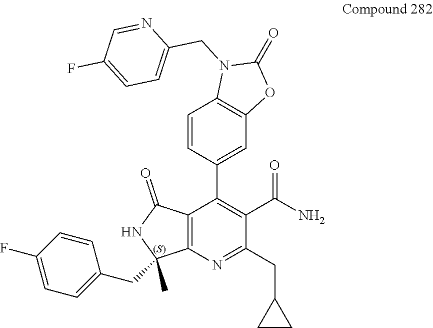

Compound 282 was synthesized following the similar route of Example 51, using 3-((5-fluoropyridin-2-yl)methyl)-2-oxo-2,3-dihydrobenzo[d]oxazole-6-carbaldehyde and(S)-4-amino-5-(4-fluorobenzyl)-5-methyl-1,5-dihydro-2H-pyrrol-2-one in step G. ¹H NMR (400 MHz, CD₃OD) δ 8.40 (d, J=2.8 Hz, 1H), 7.60 (td, J=8.4, 2.8 Hz, 1H), 7.49-7.53 (m, 1H), 6.94-7.14 (m, 3H), 6.86-6.91 (m, 2H), 6.78-6.83 (m, 2H), 5.15 (s, 2H), 3.41 (d, J=13.6 Hz, 1H), 3.14 (d, J=13.6 Hz, 1H), 2.91 (d, J=7.2 Hz, 2H), 1.68 (s, 3H), 1.35-1.42 (m, 1H), 0.54-0.62 (m, 2H), 0.36-0.47 (m, 2H). ¹⁹F NMR (376 MHz, CD₃OD) δ −118.47, −130.10. LC-MS: m/z 638.5 (M+H)⁺.

### (S)-3-[(p-fluorophenyl)methyl]-7-{3-[(5-fluoro-2-pyridyl)methyl]-2-oxo-2,3-dihydro-1,3-benzoxazolidin-6-yl}-3-methyl-1-oxo-5-(2,2,2-trifluoroethyl)-2,4-diaza-6-indancarboxamide (Compound 283)

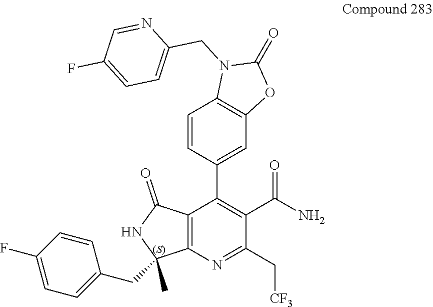

Compound 283 was synthesized following the similar route of Example 51, using 3-((5-fluoropyridin-2-yl)methyl)-2-oxo-2,3-dihydrobenzo[d]oxazole-6-carbaldehyde, (S)-4-amino-5-(4-fluorobenzyl)-5-methyl-1,5-dihydro-2H-pyrrol-2-one and tert-butyl(5,5,5-trifluoro-3-oxopentanoyl) carbamate in step G. ¹H NMR (400 MHz, CD₃OD) δ 8.40 (d, J=2.8 Hz, 1H), 7.61 (td, J=8.4, 2.4 Hz, 1H), 7.48-7.54 (m, 1H), 7.05 (d, J=8.4 Hz, 3H), 6.73-6.88 (m, 4H), 5.16 (s, 2H), 3.92-4.05 (m, 2H), 3.40 (d, J=13.2 Hz, 1H), 3.16 (d, J=13.2 Hz, 1H), 1.69 (s, 3H). ¹⁹FNMR (376 MHz, CD₃OD) δ −64.11-118.36, −130.08. LC-MS: m/z 624.2 (M+H)⁺.

### 5-(cyclopropylmethyl)-7-{4-[(5-fluoro-2-pyridyl)methyl]-2,2-dimethyl-3-oxo-3,4-dihydro-2H-1,4-benzoxazin-7-yl}-3-(2,2,2-trifluoroethyl)-1,3,4-triaza-3H-indene-6-carboxamide (Compound 284)

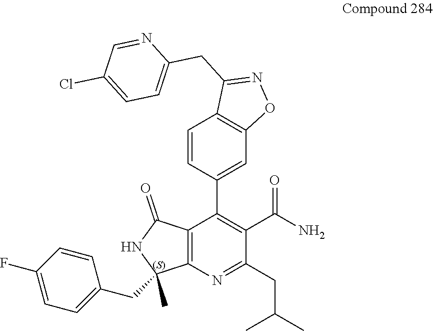

Compound 284 was synthesized following the similar route of Example 50, using 3-((5-chloropyridin-2-yl)methyl)benzo[d]isoxazole-6-carbaldehyde, (S)-4-amino-5-(4-fluorobenzyl)-5-methyl-1,5-dihydro-2H-pyrrol-2-one and 5-methyl-3-oxohexanamide in step A.

¹H NMR (400 MHz, CDCl₃) δ 8.53 (d, J=2.4 Hz, 1H), 7.62-7.66 (m, 2H), 7.53 (d, J=4.4 Hz, 1H), 7.32 (d, J=8.4 Hz, 2H), 7.00-7.04 (m, 2H), 6.89-6.93 (m, 2H), 6.13 (br s, 1H), 5.54 (br s, 1H), 5.24 (br s, 1H), 4.53 (s, 2H), 3.20-3.25 (m, 1H), 3.14 (s, 1H), 2.93 (t, J=7.6 Hz, 2H), 2.44 (dd, J=13.6, 6.8 Hz, 1H), 1.63 (br s, 3H), 1.06 (d, J=6.4 Hz, 3H), 1.00 (d, J=6.8 Hz, 3H). ¹⁹F NMR (376 MHz, CDCl₃) δ −115.38. LC-MS: m/z 598.1 (M+H)⁺.

### Example 59

### 7-{4-[(5-chloro-2-pyridyl)methyl]-2,2-dimethyl-3-oxo-3,4-dihydro-2H-1,4-benzoxazin-7-yl}-3,5-bis(cyclopropylmethyl)-3-methyl-1-oxo-2-oxa-4-aza-6-indancarboxamide (Compound 285)

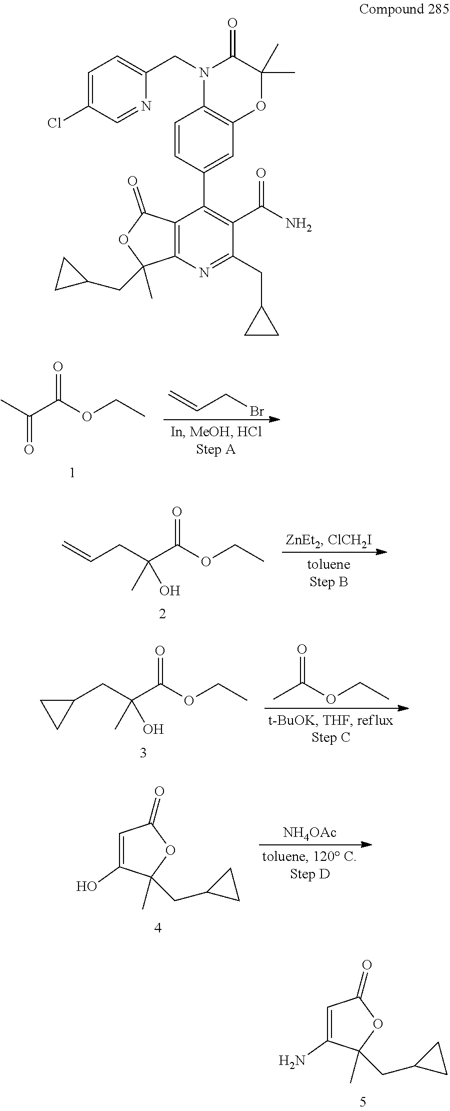

### Step A: ethyl 2-hydroxy-2-methylpent-4-enoate

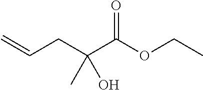

A mixture of ethyl 2-oxopropanoate (5 g, 43.06 mmol, 4.78 mL), 3-bromoprop-1-ene (7.81 g, 64.59 mmol, 5.59 mL), Indium (4.94 g, 43.06 mmol, 677.28 μL) in MeOH (10 mL) and 0.1 N HCl (40 mL) was degassed and purged with N₂ for 3 times, and then the mixture was stirred at 25° C. for 2 hr under N₂ atmosphere. The reaction mixture was partitioned between sat. NaHCO₃ aqueous (100 mL) and EtOAc (100 mL). The organic phase was separated, concentrated under reduced pressure to give a residue. The residue was purified by flash silica gel chromatography (ISCOR; 40 g SepaFlash® Silica Flash Column, Eluent of 0~10% Ethyl acetate/Petroleum ether gradient @ 30 mL/min) to afford ethyl 2-hydroxy-2-methylpent-4-enoate (5.4 g, 79.27%) as a colorless oil. ¹H NMR (400 MHz, CDCl₃) δ 5.86-5.70 (m, 1H), 5.15-5.13 (m, 1H), 5.13-5.09 (m, 1H), 4.28-4.19 (m, 2H), 3.17 (s, 1H), 2.55-2.47 (m, 1H), 2.44-2.36 (m, 1H), 1.42 (s, 3H), 1.30 (t, J=7.2 Hz, 3H).

### Step B: ethyl 3-cyclopropyl-2-hydroxy-2-methylpropanoate

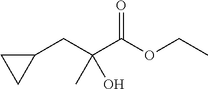

To a solution of ZnEt₂ (1 M, 18.96 mL) in toluene (5 mL) was added chloroiodomethane (6.69 g, 37.93 mmol, 2.75 mL) at −20° C., and the reaction mixture was stirred at −20° C. for 1 hr. And then ethyl 2-hydroxy-2-methylpent-4-enoate (1 g, 6.32 mmol) in toluene (3 mL) was added dropwise at −20° C. The mixture was stirred at −20° C. for 16 hr. The reaction mixture was quenched by addition of sat. NH₄Cl aqueous (20 mL) at 0° C., and then diluted with H₂O (20 mL) and extracted with EtOAc (50 mL). The organic layer was washed with brine (50 mL), dried over Na₂SO₄, filtered and concentrated under reduced pressure to afford ethyl 3-cyclopropyl-2-hydroxy-2-methylpropanoate (0.9 g, 82.67%) as a colorless oil, which was used into the next step without further purification. ¹H NMR (400 MHz, CDCl₃) δ 4.31-4.18 (m, 2H), 3.30 (s, 1H), 1.72 (dd, J=6.0, 14.0 Hz, 1H), 1.55 (dd, J=7.6, 14.0 Hz, 1H), 1.41 (s, 3H), 1.31 (t, J=7.2 Hz, 3H), 0.82-0.71 (m, 1H), 0.53-0.36 (m, 2H), 0.16-0.06 (m, 2H).

### Step C: 5-(cyclopropylmethyl)-4-hydroxy-5-methylfuran-2 (5H)-one

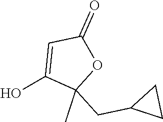

To a solution of ethyl 3-cyclopropyl-2-hydroxy-2-methylpropanoate (0.7 g, 4.06 mmol) and EtOAc (716.21 mg, 8.13 mmol, 795.79 μL) in THF (10 mL) was added t-BuOK (912.18 mg, 8.13 mmol) at 0° C. Then the mixture was stirred at 0-60° C. for 16 hr. The reaction mixture was quenched by addition H₂O (20 mL) at 25° C., and then extracted with EA (20 mL*2). The aqueous phase was adjusted pH~3 and extracted with EtOAc (40 mL). The organic layer was concentrated under reduced pressure to give a residue. The residue was purified by flash silica gel chromatography (Silica gel, Eluent of 0~40% Ethyl acetate in Petroleum ether, gradient @ 20 mL/min) to afford 5-(cyclopropylmethyl)-4-hydroxy-5-methylfuran-2 (5H)-one (150 mg, 21.94%) as a colorless oil. LC-MS: m/z 169.1 (M+H)⁺.

### Step D: 4-amino-5-(cyclopropylmethyl)-5-methylfuran-2 (5H)-one

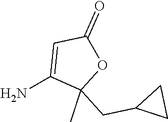

To a solution of 5-(cyclopropylmethyl)-4-hydroxy-5-methylfuran-2 (5H)-one (150 mg, 891.85 μmol) in toluene (3 mL) was added NH₄OAc (274.98 mg, 3.57 mmol). The mixture was stirred at 120° C. for 1.5 hr. The reaction mixture was concentrated under reduced pressure to afford 4-amino-5-(cyclopropylmethyl)-5-methylfuran-2 (5H)-one (145 mg, 97.24%) as a brown oil, which was used into the next step without further purification. LC-MS: m/z 168.1 (M+H)⁺.

### 7-{4-[(5-chloro-2-pyridyl)methyl]-2,2-dimethyl-3-oxo-3,4-dihydro-2H-1,4-benzoxazin-7-yl}-3,5-bis(cyclopropylmethyl)-3-methyl-1-oxo-2-oxa-4-aza-6-indancarboxamide (Compound 285)

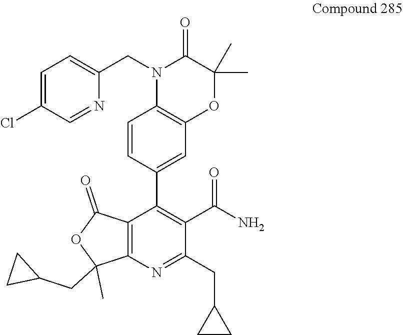 [SMILES: CC1(C)Oc2cc(-c3c(C(N)=O)c(CC4CC4)nc4c3C(=O)OC4(C)CC3CC3)ccc2N(Cc2ccc(Cl)cn2)C1=O]

Compound 285 was then synthesized following the similar route of Example 51, using 4-amino-5-(cyclopropylmethyl)-5-methylfuran-2 (5H)-one and 4-((5-chloropyridin-2-yl)methyl)-2,2-dimethyl-3-oxo-3,4-dihydro-2H-benzo[b][1,4]oxazine-7-carbaldehyde in step G. ¹H NMR (400 MHz, CD₃OD) δ 8.53 (d, J=2.4 Hz, 1H), 7.84 (dd, J=2.4, 8.4 Hz, 1H), 7.34 (d, J=8.4 Hz, 1H), 7.14 (s, 1H), 7.07 (d, J=0.8 Hz, 2H), 5.29 (s, 2H), 2.90 (dd, J=2.0, 7.2 Hz, 2H), 2.16-2.07 (m, 1H), 2.02-1.93 (m, 1H), 1.72 (s, 3H), 1.58 (s, 6H), 1.34-1.28 (m, 1H), 0.56-0.44 (m, 3H), 0.44-0.33 (m, 3H), 0.24-0.10 (m, 2H), −0.12-−0.20 (m, 1H). LC-MS: m/z 601.1 (M+H)⁺.

### (R)-7-{4-[(5-chloro-2-pyridyl)methyl]-2,2-dimethyl-3-oxo-3,4-dihydro-2H-1,4-benzoxazin-7-yl}-3,5-bis(cyclopropylmethyl)-3-methyl-1-oxo-2-oxa-4-aza-6-indancarboxamide and(S)-7-{4-[(5-chloro-2-pyridyl)methyl]-2,2-dimethyl-3-oxo-3,4-dihydro-2H-1,4-benzoxazin-7-yl}-3,5-bis(cyclopropylmethyl)-3-methyl-1-oxo-2-oxa-4-aza-6-indancarboxamide (Compound 286 and Compound 287)

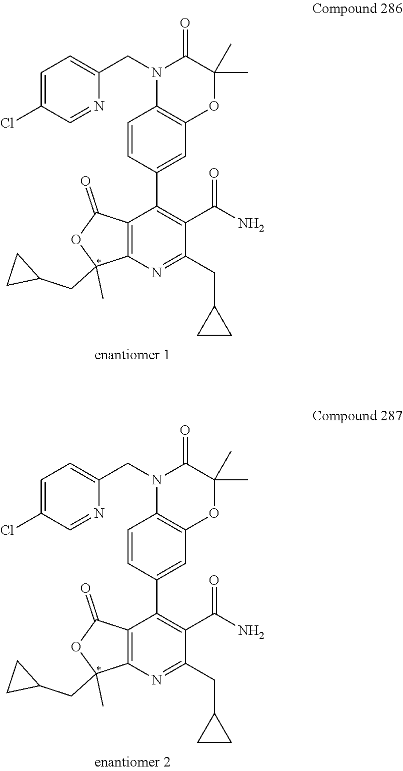 [SMILES: CC1(C)Oc2cc(-c3c(C(N)=O)c(CC4CC4)nc4c3C(=O)OC4(C)CC3CC3)ccc2N(Cc2ccc(Cl)cn2)C1=O ; CC1(C)Oc2cc(-c3c(C(N)=O)c(CC4CC4)nc4c3C(=O)OC4(C)CC3CC3)ccc2N(Cc2ccc(Cl)cn2)C1=O]

7-{4-[(5-chloro-2-pyridyl)methyl]-2,2-dimethyl-3-oxo-3,4-dihydro-2H-1,4-benzoxazin-7-yl}-3,5-bis(cyclopropylmethyl)-3-methyl-1-oxo-2-oxa-4-aza-6-indancarboxamide (100 mg) was purified by SFC (system: SFC 27; Column name: ChiralPak IH; Column size: (250*30 mm, 10 μm); mobile phase A: Supercritical CO₂; mobile phase B: EtOH (0.1% NH₃H₂O); Gradient: A/B=50/50; Flow rate: 150 mL/min; Column Temp: RT) to afford (R)-7-{4-[(5-chloro-2-pyridyl)methyl]-2,2-dimethyl-3-oxo-3,4-dihydro-2H-1,4-benzoxazin-7-yl}-3,5-bis(cyclopropylmethyl)-3-methyl-1-oxo-2-oxa-4-aza-6-indancarboxamide (35.91 mg) and(S)-7-{4-[(5-chloro-2-pyridyl)methyl]-2,2-dimethyl-3-oxo-3,4-dihydro-2H-1,4-benzoxazin-7-yl}-3,5-bis(cyclopropylmethyl)-3-methyl-1-oxo-2-oxa-4-aza-6-indancarboxamide (43.28 mg).

Compound 286, enantiomer 1: Rt=1.75 min. ¹H NMR (400 MHz, CD₃OD) δ 8.53 (d, J=2.4 Hz, 1H), 7.84 (dd, J=2.4, 8.4 Hz, 1H), 7.34 (d, J=8.4 Hz, 1H), 7.14 (s, 1H), 7.07 (d, J=1.2 Hz, 2H), 5.29 (s, 2H), 2.90 (dd, J=2.4, 7.2 Hz, 2H), 2.16-2.08 (m, 1H), 2.02-1.93 (m, 1H), 1.72 (s, 3H), 1.58 (s, 6H), 1.35-1.29 (m, 1H), 0.56-0.46 (m, 3H), 0.44-0.35 (m, 3H), 0.24-0.11 (m, 2H), −0.12-−0.20 (m, 1H). LC-MS: m/z 601.1 (M−H)⁺.

Compound 287, enantiomer 2: Rt=2.09 min. ¹H NMR (400 MHz, CD₃OD) δ 8.53 (d, J=2.4 Hz, 1H), 7.84 (dd, J=2.4, 8.4 Hz, 1H), 7.34 (d, J=8.4 Hz, 1H), 7.14 (s, 1H), 7.07 (d, J=0.8 Hz, 2H), 5.29 (s, 2H), 2.90 (dd, J=2.4, 6.8 Hz, 2H), 2.17-2.07 (m, 1H), 2.02-1.92 (m, 1H), 1.72 (s, 3H), 1.58 (s, 6H), 1.33-1.29 (m, 1H), 0.56-0.45 (m, 3H), 0.44-0.34 (m, 3H), 0.25-0.11 (m, 2H), −0.12-−0.21 (m, 1H). LC−MS: m/z 601.1 (M−H)⁺.

### (R)-7-{4-[(5-chloro-2-pyrimidinyl)methyl]-2,2-dimethyl-3-oxo-3,4-dihydro-2H-1,4-benzoxazin-7-yl}-3,5-bis(cyclopropylmethyl)-3-methyl-1-oxo-2-oxa-4-aza-6-indancarboxamide and(S)-7-{4-[(5-chloro-2-pyrimidinyl)methyl]-2,2-dimethyl-3-oxo-3,4-dihydro-2H-1,4-benzoxazin-7-yl}-3,5-bis(cyclopropylmethyl)-3-methyl-1-oxo-2-oxa-4-aza-6-indancarboxamide (Compound 288 and Compound 289)

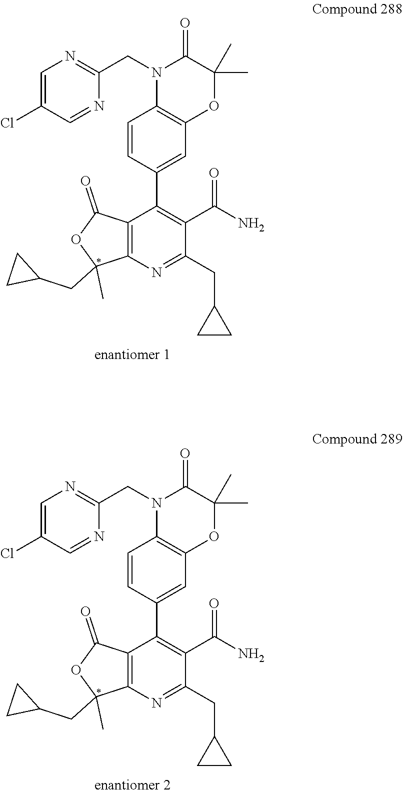 [SMILES: CC1(C)Oc2cc(-c3c(C(N)=O)c(CC4CC4)nc4c3C(=O)OC4(C)CC3CC3)ccc2N(Cc2ncc(Cl)cn2)C1=O ; CC1(C)Oc2cc(-c3c(C(N)=O)c(CC4CC4)nc4c3C(=O)OC4(C)CC3CC3)ccc2N(Cc2ncc(Cl)cn2)C1=O]

7-{4-[(5-chloro-2-pyrimidinyl)methyl]-2,2-dimethyl-3-oxo-3,4-dihydro-2H-1,4-benzoxazin-7-yl}-3,5-bis(cyclopropylmethyl)-3-methyl-1-oxo-2-oxa-4-aza-6-indancarboxamide which was synthesized following the similar route of Example 51, using 4-amino-5-(cyclopropylmethyl)-5-methylfuran-2 (5H)-one and 4-((5-chloropyrimidin-2-yl)methyl)-2,2-dimethyl-3-oxo-3,4-dihydro-2H-benzo[b][1,4]oxazine-7-carbaldehyde in step G (170 mg) was purified by SFC (system: SFC 27; Column name: ChiralPak IH; Column size: (250*30 mm, 10 μm); mobile phase A: Supercritical CO₂; mobile phase B: EtOH (0.1% NH₃H₂O); Gradient: A/B=50/50; Flow rate: 150 mL/min; Column Temp: RT) to afford (R)-7-{4-[(5-chloro-2-pyrimidinyl)methyl]-2,2-dimethyl-3-oxo-3,4-dihydro-2H-1,4-benzoxazin-7-yl}-3,5-bis(cyclopropylmethyl)-3-methyl-1-oxo-2-oxa-4-aza-6-indancarboxamide (62.8 mg) and(S)-7-{4-[(5-chloro-2-pyrimidinyl)methyl]-2,2-dimethyl-3-oxo-3,4-dihydro-2H-1,4-benzoxazin-7-yl}-3,5-bis(cyclopropylmethyl)-3-methyl-1-oxo-2-oxa-4-aza-6-indancarboxamide (68.54 mg).

Compound 288, enantiomer 1: Rt-2.08 min. ¹H NMR (400 MHz, CD₃OD) δ 8.76 (s, 2H), 7.13 (d, J=2.0 Hz, 1H), 7.04 (dd, J=2.0, 8.4 Hz, 1H), 6.90 (d, J=8.4 Hz, 1H), 5.36 (s, 2H), 2.88 (dd, J=2.4, 7.2 Hz, 2H), 2.09 (dd, J=7.2, 14.8 Hz, 1H), 1.95 (dd, J=7.2, 14.8 Hz, 1H), 1.70 (s, 3H), 1.56 (s, 6H), 1.33-1.27 (m, 1H), 0.55-0.45 (m, 3H), 0.41-0.33 (m, 3H), 0.21-0.09 (m, 2H), −0.14-−0.22 (m, 1H). LC-MS: m/z 602.0 (M−H)⁺.

Compound 289, enantiomer 2: Rt=2.40 min. ¹H NMR (400 MHz, CD₃OD) δ 8.76 (s, 2H), 7.13 (d, J=2.0 Hz, 1H), 7.04 (dd, J=2.0, 8.4 Hz, 1H), 6.90 (d, J=8.4 Hz, 1H), 5.36 (s, 2H), 2.88 (dd, J=2.4, 7.2 Hz, 2H), 2.14-2.06 (m, 1H), 1.99-1.92 (m, J=7.2, 14.4 Hz, 1H), 1.70 (s, 3H), 1.56 (s, 6H), 1.33-1.27 (m, 1H), 0.54-0.45 (m, 3H), 0.41-0.33 (m, 3H), 0.22-0.09 (m, 2H), −0.14-−0.23 (m, 1H). LC-MS: m/z 602.1 (M+H)⁺.

### 7-{4-[(5-chloro-2-pyridyl)methyl]-2,2-difluoro-3-oxo-3,4-dihydro-2H-1,4-benzoxazin-7-yl}-3,5-bis(cyclopropylmethyl)-3-methyl-1-oxo-2-oxa-4-aza-6-indancarboxamide (Compound 290)

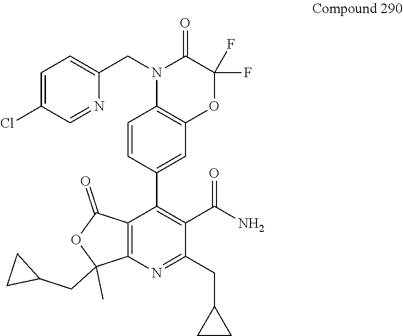 [SMILES: CC1(CC2CC2)OC(=O)c2c1nc(CC1CC1)c(C(N)=O)c2-c1ccc2c(c1)OC(F)(F)C(=O)N2Cc1ccc(Cl)cn1]

Compound 290 was synthesized following the similar route of Example 51, using 4-amino-5-(cyclopropylmethyl)-5-methylfuran-2 (5H)-one and 4-((5-chloropyridin-2-yl)methyl)-2,2-difluoro-3-oxo-3,4-dihydro-2H-benzo[b][1,4]oxazine-7-carbaldehyde in step G. ¹H NMR (400 MHz, CD₃OD) δ 8.52 (d, J=2.0 Hz, 1H), 7.86 (dd, J=2.4, 8.4 Hz, 1H), 7.45 (d, J=8.8 Hz, 1H), 7.40 (d, J=1.2 Hz, 1H), 7.35-7.26 (m, 2H), 5.41 (s, 2H), 2.92-2.86 (m, 2H), 2.15-2.07 (m, 1H), 2.00-1.91 (m, 1H), 1.71 (s, 3H), 1.33-1.26 (m, 1H), 0.55-0.45 (m, 3H), 0.41-0.32 (m, 3H), 0.23-0.09 (m, 2H), −0.13-−0.22 (m, 1H). ¹⁹F NMR (376 MHz, CD₃OD) δ −78.19. LC-MS: m/z 609.0 (M+H)⁺.

### (R)-7-{4-[(5-chloro-2-pyrimidinyl)methyl]-2,2-difluoro-3-oxo-3,4-dihydro-2H-1,4-benzoxazin-7-yl}-3,5-bis(cyclopropylmethyl)-3-methyl-1-oxo-2-oxa-4-aza-6-indancarboxamide and(S)-7-{4-[(5-chloro-2-pyrimidinyl)methyl]-2,2-difluoro-3-oxo-3,4-dihydro-2H-1,4-benzoxazin-7-yl}-3,5-bis(cyclopropylmethyl)-3-methyl-1-oxo-2-oxa-4-aza-6-indancarboxamide (Compound 291 and Compound 292)

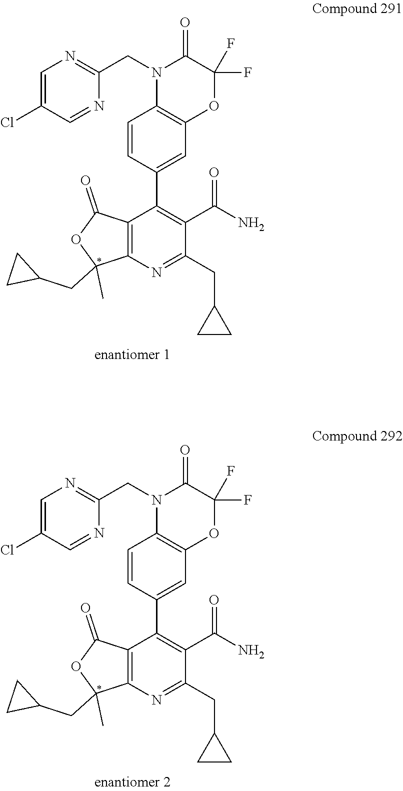 [SMILES: CC1(CC2CC2)OC(=O)c2c1nc(CC1CC1)c(C(N)=O)c2-c1ccc2c(c1)OC(F)(F)C(=O)N2Cc1ncc(Cl)cn1 ; CC1(CC2CC2)OC(=O)c2c1nc(CC1CC1)c(C(N)=O)c2-c1ccc2c(c1)OC(F)(F)C(=O)N2Cc1ncc(Cl)cn1]

7-{4-[(5-chloro-2-pyrimidinyl)methyl]-2,2-difluoro-3-oxo-3,4-dihydro-2H-1,4-benzoxazin-7-yl}-3,5-bis(cyclopropylmethyl)-3-methyl-1-oxo-2-oxa-4-aza-6-indancarboxamide which was synthesized following the similar route of Example 51, using 4-amino-5-(cyclopropylmethyl)-5-methylfuran-2 (5H)-one in step G (150 mg) was purified by SFC (system: Waters SFC 27; Column name: ChiralPak IH; Column size: (250 mm*30 mm, 10 μm); mobile phase A: CO₂; mobile phase B: EtOH (+0.1% NH₃H₂O); Gradient: A/B=50/50; Flow rate: 150 mL/min; Column Temp: RT) to afford (R)-7-{4-[(5-chloro-2-pyrimidinyl)methyl]-2,2-difluoro-3-oxo-3,4-dihydro-2H-1,4-benzoxazin-7-yl}-3,5-bis(cyclopropylmethyl)-3-methyl-1-oxo-2-oxa-4-aza-6-indancarboxamide (18.43 mg) and(S)-7-{4-[(5-chloro-2-pyrimidinyl)methyl]-2,2-difluoro-3-oxo-3,4-dihydro-2H-1,4-benzoxazin-7-yl}-3,5-bis(cyclopropylmethyl)-3-methyl-1-oxo-2-oxa-4-aza-6-indancarboxamide (27.37 mg).

Compound 291, enantiomer 1: Rt=1.76 min. ¹H NMR (400 MHz, CD₃OD) δ 8.79 (s, 2H), 7.41 (d, J=1.6 Hz, 1H), 7.27 (dd, J=8.4, 1.6 Hz, 1H), 7.18-7.21 (m, 1H), 5.52 (s, 2H), 2.89 (dd, J=7.0, 1.8 Hz, 2H), 2.07-2.14 (m, 1H), 1.95 (dd, J=14.8, 7.6 Hz, 1H), 1.71 (s, 3H), 1.16-1.21 (m, 1H), 0.35-0.42 (m, 3H), 0.21-0.29 (m, 3H), −0.01-0.10 (m, 2H), −0.23-−0.14 ppm (m, 1H). ¹⁹F NMR (376 MHz, CD₃OD) δ −78.28. LC-MS: m/z 610.1 (M+H)⁺.

Compound 292, enantiomer 2: Rt=2.16 min. ¹H NMR (400 MHz, CD₃OD) δ 8.79 (s, 2H), 7.41 (d, J=2.0 Hz, 1H), 7.26-7.29 (m, 1H), 7.18-7.20 (m, 1H), 5.52 (s, 2H), 2.89 (dd, J=7.2, 2.0 Hz, 2H), 2.07-2.14 (m, 1H), 1.95 (dd, J=14.5, 7.5 Hz, 1H), 1.71 (s, 3H), 1.10-1.26 (m, 1H), 0.35-0.42 (m, 3H), 0.21-0.29 (m, 3H), −0.01-0.08 (m, 2H), −0.20-−0.15 (m, 1H). ¹⁹F NMR (376 MHz, CD₃OD) δ −78.32. LC-MS: m/z 610.1 (M+H)⁺.

### 7-{4-[(5-chloro-2-pyridyl)methyl]-2,2-dimethyl-3-oxo-3,4-dihydro-2H-1,4-benzoxazin-7-yl}-3-cyclopropyl-5-(cyclopropylmethyl)-3-methyl-1-oxo-2-oxa-4-aza-6-indancarboxamide (Compound 293)

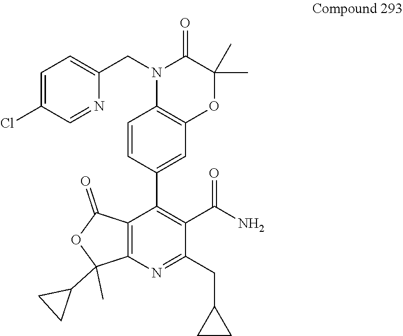 [SMILES: CC1(C)Oc2cc(-c3c(C(N)=O)c(CC4CC4)nc4c3C(=O)OC4(C)C3CC3)ccc2N(Cc2ccc(Cl)cn2)C1=O]

Compound 293 was then synthesized following the similar route of Example 59, using 2-cyclopropyl-2-hydroxypropanoic acid in step A. ¹H NMR (400 MHz, CD₃OD) δ 8.51 (d, J=2.4 Hz, 1H), 7.82 (dd, J=2.4, 8.4 Hz, 1H), 7.32 (d, J=8.4 Hz, 1H), 7.11 (s, 1H), 7.04 (d, J=0.8 Hz, 2H), 5.27 (s, 2H), 2.88 (dd, J=1.6, 7.2 Hz, 2H), 1.76 (s, 3H), 1.56 (s, 6H), 1.35-1.30 (m, 1H), 0.65-0.57 (m, 1H), 0.55-0.32 (m, 8H). LC-MS: m/z 587.1 (M+H)⁺.

### Example 60

### 7-{4-[(5-chloro-2-pyridyl)methyl]-2,2-dimethyl-3-oxo-3,4-dihydro-2H-1,4-benzoxazin-7-yl}-5-(cyclopropylmethyl)-3-methyl-1-oxo-3-(2,2,2-trifluoroethyl)-2-oxa-4-aza-6-indancarboxamide (Compound 294)

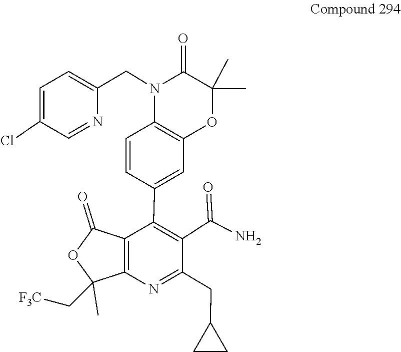 [SMILES: CC1(C)Oc2cc(-c3c(C(N)=O)c(CC4CC4)nc4c3C(=O)OC4(C)CC(F)(F)F)ccc2N(Cc2ccc(Cl)cn2)C1=O]

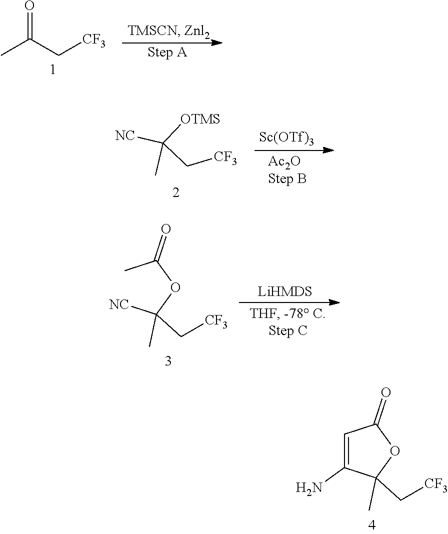

### Step A: 4,4,4-trifluoro-2-methyl-2-trimethylsilyloxy-butanenitrile

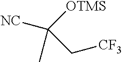

To a solution of 4,4,4-trifluorobutan-2-one (880 mg, 6.98 mmol) in DCM (15 mL) was added diiodozine (668.39 mg, 2.09 mmol) and TMSCN (1.04 g, 10.47 mmol, 1.31 mL) at 0° C. under N₂ atmosphere. The mixture was stirred at 25° C. for 16 hr under N₂ atmosphere. The mixture was diluted with H₂O (40 mL), extracted with DCM (20 mL×3), the combined organic layers were washed with brine (40 mL), dried over Na₂SO₄, filtered and concentrated to afford 4,4,4-trifluoro-2-methyl-2-trimethylsilyloxy-butanenitrile (1.23 g, crude) as a brown oil which was used into the next step without further purification. ¹H NMR (400 MHz, CDCl₃) δ 2.52-2.66 (m, 2H), 1.71 (s, 3H), 0.25-0.33 (m, 9H).

### Step B: 2-cyano-4,4,4-trifluorobutan-2-yl acetate

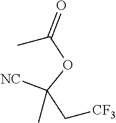

To a solution of 4,4,4-trifluoro-2-methyl-2-trimethylsilyloxy-butanenitrile (1.11 g, 4.93 mmol) in MeCN (10 mL) was added Ac₂O (1.01 g, 9.85 mmol, 925.49 μL) and followed by scandium (IV) trifluoromethanesulfonate (242.50 mg, 492.71 μmol). The mixture was stirred at 25° C. for 16 hr. The mixture was concentrated to give a residue. The residue was purified by flash silica gel chromatography (PE:EA=1:0 to 3:2) to afford (1-cyano-3,3,3-trifluoro-1-methyl-propyl)acetate (383 mg, 39.83) as a pale-yellow oil. ¹H NMR (400 MHz, CDCl₃) δ 2.79-3.08 (m, 2H), 2.13 (s, 3H), 1.90 (s, 3H).

### Step C: 4-amino-5-methyl-5-(2,2,2-trifluoroethyl) furan-2 (5H)-one

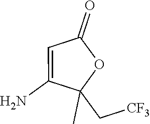

To a solution of (1-cyano-3,3,3-trifluoro-1-methyl-propyl)acetate (383 mg, 1.96 mmol) in THF (6 mL) was added dropwise LiHMDS (1 M, 3.93 mL) at −78° C. under N₂ atmosphere. The mixture was stirred at −78° C. for 0.5 hr under N₂ atmosphere. Then the mixture was stirred at −40° C. for 1.5 hr under N₂ atmosphere. The mixture was quenched with sat. NH₄Cl (40 mL), extracted with EA (20 mL×3), the combined organic layers were washed with brine (20 mL), dried over Na₂SO₄, filtered and concentrated to afford 4-amino-5-methyl-5-(2,2,2-trifluoroethyl) furan-2 (5H)-one (353 mg, crude) as a brown oil which was used into the next step without further purification. ¹H NMR (400 MHz, CDCl₃) δ 4.91 (br s, 2H), 2.52-2.82 (m, 2H), 1.60 (s, 3H).

### 7-{4-[(5-chloro-2-pyridyl)methyl]-2,2-dimethyl-3-oxo-3,4-dihydro-2H-1,4-benzoxazin-7-yl}-5-(cyclopropylmethyl)-3-methyl-1-oxo-3-(2,2,2-trifluoroethyl)-2-oxa-4-aza-6-indancarboxamide (Compound 294)

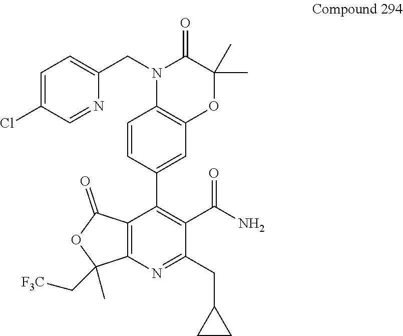 [SMILES: CC1(C)Oc2cc(-c3c(C(N)=O)c(CC4CC4)nc4c3C(=O)OC4(C)CC(F)(F)F)ccc2N(Cc2ccc(Cl)cn2)C1=O]

Compound 294 was then synthesized following the similar route of Example 51, using 4-amino-5-methyl-5-(2,2,2-trifluoroethyl) furan-2 (5H)-one and 4-((5-chloropyridin-2-yl)methyl)-2,2-dimethyl-3-oxo-3,4-dihydro-2H-benzo[b][1,4]oxazine-7-carbaldehyde in step G. ¹H NMR (400 MHz, CD₃OD) δ 8.51 (d, J=2.4 Hz, 1H), 7.82 (dd, J=8.4, 2.4 Hz, 1H), 7.33 (d, J=8.4 Hz, 1H), 7.12 (s, 1H), 7.06 (d, J=0.8 Hz, 2H), 5.28 (s, 2H), 3.06-3.18 (m, 2H), 2.89 (dd, J=6.8, 1.6 Hz, 2H), 1.76 (s, 3H), 1.56 (s, 6H), 1.28-1.32 (m, 1H), 0.47-0.55 (m, 2H), 0.31-0.37 (m, 2H). ¹⁹F NMR (376 MHz, CD₃OD) δ −62.62. LC-MS: m/z 629.1 (M+H)⁺.

### Example 61

### 4-{2,2-difluoro-4-[(5-fluoro-2-pyrimidinyl)methyl]-3-oxo-3,4-dihydro-2H-1,4-benzoxazin-7-yl}-1-[1-(5-fluoro-2-pyridyl)cyclopropyl]-6-[(1-hydroxycyclopropyl)methyl]-1,2,7-triaza-1H-indene-5-carboxamide (Compound 295)

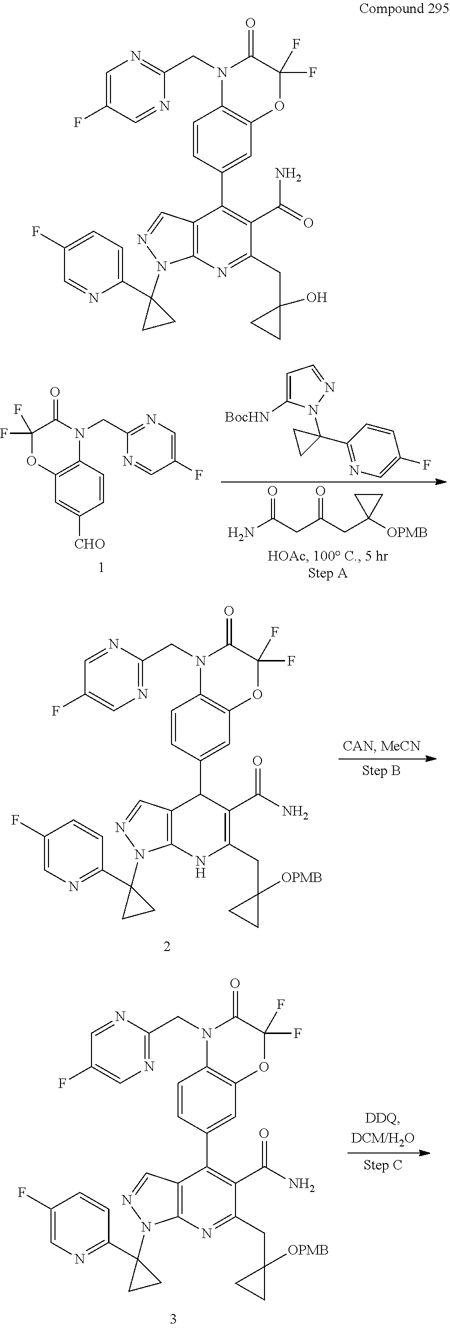

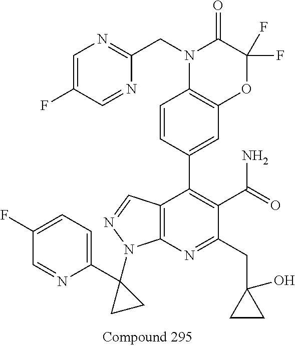 [SMILES: NC(=O)c1c(CC2(O)CC2)nc2c(cnn2C2(c3ccc(F)cn3)CC2)c1-c1ccc2c(c1)OC(F)(F)C(=O)N2Cc1ncc(F)cn1]

### Step A: 4-(2,2-difluoro-4-((5-fluoropyrimidin-2-yl)methyl)-3-oxo-3,4-dihydro-2H-benzo[b][1,4]oxazin-7-yl)-1-(1-(5-fluoropyridin-2-yl)cyclopropyl)-6-((1-((4-methoxybenzyl)oxy)cyclopropyl)methyl)-4,7-dihydro-1H-pyrazolo[3,4-b]pyridine-5-carboxamide

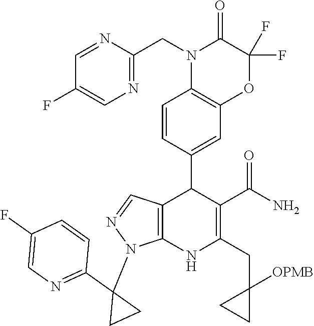

To a solution of 2,2-difluoro-4-((5-fluoropyrimidin-2-yl)methyl)-3-oxo-3,4-dihydro-2H-benzo[b][1,4]oxazine-7-carbaldehyde (150 mg, 464.07 μmol) in AcOH (5 mL) was added 4-(1-((4-methoxybenzyl)oxy)cyclopropyl)-3-oxobutanamide (260 mg, 937.56 μmol), tert-butyl(1-(1-(5-fluoropyridin-2-yl)cyclopropyl)-1H-pyrazol-5-yl)carbamate (160 mg, 502.60 μmol) at 10-15° C. After addition, the mixture was stirred at 100° C. for 5 hours. The reaction mixture was concentrated to afford 4-(2,2-difluoro-4-((5-fluoropyrimidin-2-yl)methyl)-3-oxo-3,4-dihydro-2H-benzo[b][1,4]oxazin-7-yl)-1-(1-(5-fluoropyridin-2-yl)cyclopropyl)-6-((1-((4-methoxybenzyl)oxy)cyclopropyl)methyl)-4,7-dihydro-1H-pyrazolo[3,4-b]pyridine-5-carboxamide (570 mg, crude) as a yellow oil. LC-MS: m/z 783.4 (M+H)⁺.

### Step B: 4-(2,2-difluoro-4-((5-fluoropyrimidin-2-yl)methyl)-3-oxo-3,4-dihydro-2H-benzo[b][1,4]oxazin-7-yl)-1-(1-(5-fluoropyridin-2-yl)cyclopropyl)-6-((1-((4-methoxybenzyl)oxy)cyclopropyl)methyl)-1H-pyrazolo[3,4-b]pyridine-5-carboxamide

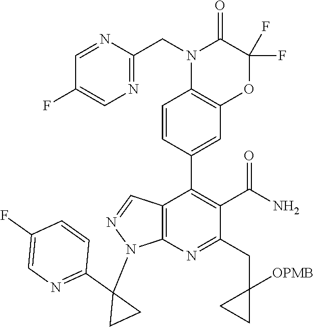

To a solution of 4-(2,2-difluoro-4-((5-fluoropyrimidin-2-yl)methyl)-3-oxo-3,4-dihydro-2H-benzo[b][1,4]oxazin-7-yl)-1-(1-(5-fluoropyridin-2-yl)cyclopropyl)-6-((1-((4-methoxybenzyl)oxy)cyclopropyl)methyl)-4,7-dihydro-1H-pyrazolo[3,4-b]pyridine-5-carboxamide (570 mg, 458.77 mol) in ACN (10 mL) was added CAN (250 mg, 456.02 μmol). The mixture was stirred at 15-20° C. for 0.5 hours. The reaction mixture was concentrated to give the crude product which was purified by column chromatography (SiO₂, 3% methanol in dichloromethane) to afford 4-(2,2-difluoro-4-((5-fluoropyrimidin-2-yl)methyl)-3-oxo-3,4-dihydro-2H-benzo[b][1,4]oxazin-7-yl)-1-(1-(5-fluoropyridin-2-yl)cyclopropyl)-6-((1-((4-methoxybenzyl)oxy)cyclopropyl)methyl)-1H-pyrazolo[3,4-b]pyridine-5-carboxamide (380 mg, 50.92%) as a yellow oil. LC-MS: m/z 781.5 (M+H)⁺.

### Step C: 4-{2,2-difluoro-4-[(5-fluoro-2-pyrimidinyl)methyl]-3-oxo-3,4-dihydro-2H-1,4-benzoxazin-7-yl}-1-[1-(5-fluoro-2-pyridyl)cyclopropyl]-6-[(1-hydroxycyclopropyl)methyl]-1,2,7-triaza-1H-indene-5-carboxamide (Compound 295)

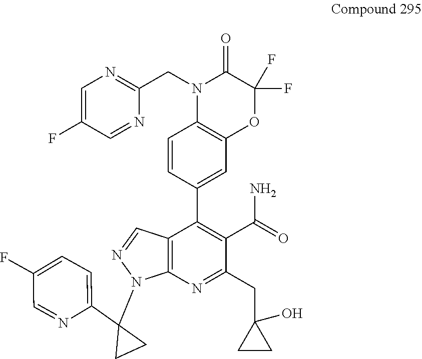 [SMILES: NC(=O)c1c(CC2(O)CC2)nc2c(cnn2C2(c3ccc(F)cn3)CC2)c1-c1ccc2c(c1)OC(F)(F)C(=O)N2Cc1ncc(F)cn1]

To a solution of 4-(2,2-difluoro-4-((5-fluoropyrimidin-2-yl)methyl)-3-oxo-3,4-dihydro-2H-benzo[b][1,4]oxazin-7-yl)-1-(1-(5-fluoropyridin-2-yl)cyclopropyl)-6-((1-((4-methoxybenzyl)oxy)cyclopropyl)methyl)-1H-pyrazolo[3,4-b]pyridine-5-carboxamide (350 mg, 215.18 umol) in DCM (10 mL) and H₂O (1 mL) was added DDQ (100 mg, 440.52 μmol) in positions. The mixture was stirred at 15-25° C. for 1 hour. The reaction mixture was concentrated and diluted with saturated NaHCO₃ ((40 mL) and water (80 mL), extracted with ethyl acetate (80 mL×4). The organic layers were washed with saturated brine (200 mL), dried over anhydrous sodium sulfate, filtered and concentrated to give the crude product. The crude product was further purified by prep-HPLC to afford 4-{2,2-difluoro-4-[(5-fluoro-2-pyrimidinyl)methyl]-3-oxo-3,4-dihydro-2H-1,4-benzoxazin-7-yl}-1-[1-(5-fluoro-2-pyridyl)cyclopropyl]-6-[(1-hydroxycyclopropyl)methyl]-1,2,7-triaza-1H-indene-5-carboxamide (34.47 mg, 23.91%).

¹H NMR (400 MHz, CDCl₃) § 8.62 (s, 2H), 8.35 (d, J=2.8 Hz, 1H), 8.01 (s, 1H), 7.57 (s, 1H), 7.47-7.40 (m, 1H), 7.20-7.12 (m, 1H), 7.10 (d, J=8.4 Hz, 1H), 6.43 (dd, J=8.8, 4.0 Hz, 1H), 5.68 (br s, 1H), 5.55 (br s, 1H), 5.49 (s, 2H), 3.25 (s, 2H), 2.09-2.05 (m, 2H), 1.90-1.86 (m, 2H), 0.85-0.75 (m, 2H), 0.60-0.51 (m, 2H). ¹⁹F NMR (376 MHz, CDCl₃) δ −76.74, −130.67, −137.74. LC-MS: m/z 661.2 (M+H)⁺.

### Example 62

### 6-(cyclopropylmethyl)-4-{2,2-difluoro-4-[(5-fluoro-2-pyrimidinyl)methyl]-3-oxo-3,4-dihydro-2H-1,4-benzoxazin-7-yl}-1-[1-(5-fluoro-2-pyridyl)ethyl]-1,7-diaza-1H-indene-5-carboxamide (Compound 296)

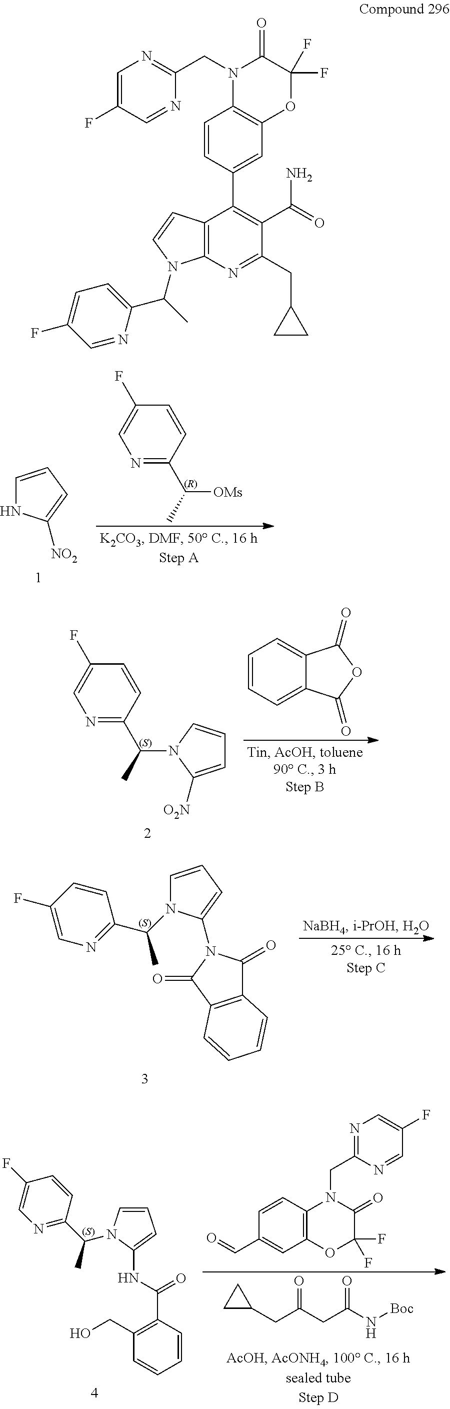

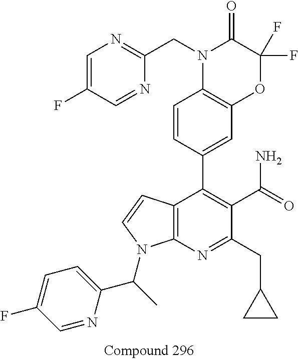 [SMILES: CC(c1ccc(F)cn1)n1ccc2c(-c3ccc4c(c3)OC(F)(F)C(=O)N4Cc3ncc(F)cn3)c(C(N)=O)c(CC3CC3)nc21]

### Step A: (S)-5-fluoro-2-(1-(2-nitro-1H-pyrrol-1-yl)ethyl)pyridine

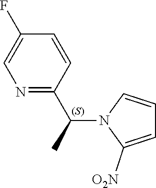

To a solution of 2-nitro-1H-pyrrole (250 mg, 2.230 mmol) and (R)-1-(5-fluoropyridin-2-yl)ethyl methanesulfonate (977.92 mg, 4.461 mmol) in DMF (20 mL) was added K₂CO₃ (1541.17 mg, 11.152 mmol). The reaction mixture was stirred at 50° C. for 16 h under N₂. The mixture was added to H₂O (50 mL) and extracted with EA (50 mL×2). The combined organics were washed with brine (50 mL) and dried over sodium sulfate, filtered and concentrated under vacuum. The resulting residue was purified by prep-TLC (PE/EA=5/1) to afford (S)-5-fluoro-2-(1-(2-nitro-1H-pyrrol-1-yl)ethyl)pyridine (476 mg, 90.54%) as a yellow oil. LC-MS: m/z 236.5 (M+H)⁺.

### Step B: (S)-2-(1-(1-(5-fluoropyridin-2-yl)ethyl)-1H-pyrrol-2-yl) isoindoline-1,3-dione

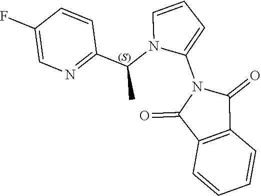

To a mixture of(S)-5-fluoro-2-(1-(2-nitro-1H-pyrrol-1-yl)ethyl)pyridine (476 mg, 2.024 mmol) in toluene (10 mL) were added 2-benzofuran-1,3-dione (599.48 mg, 4.047 mmol), Sn (1201.13 mg, 10.118 mmol) and AcOH (2.5 mL). The reaction mixture was stirred at 90° C. for 3 h under N₂. The reaction mixture was cooled to 25° C. and EA (100 mL) was added. The mixture was filtered by Celite. The cake was washed with EA (20 mL). The filtrate was concentrated under vacuum to afford (S)-2-(1-(1-(5-fluoropyridin-2-yl)ethyl)-1H-pyrrol-2-yl)isoindoline-1,3-dione (600 mg, 88.42%) as a yellow solid. LC-MS: m/z 336.6 (M+H)⁺.

### Step C: (S)—N-(1-(1-(5-fluoropyridin-2-yl)ethyl)-1H-pyrrol-2-yl)-2-(hydroxymethyl)benzamide

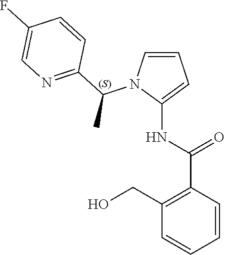

To a solution of(S)-2-(1-(1-(5-fluoropyridin-2-yl)ethyl)-1H-pyrrol-2-yl) isoindoline-1,3-dione (600 mg, 1.789 mmol) in propan-2-ol (10 mL) and H₂O (10 mL) was added NaBH₄ (270.75 mg, 7.157 mmol) at 0° C. The reaction mixture was allowed to warm to 25° C. and stirred at 25° C. for 16 h under N₂. The reaction mixture was quenched by MeOH (2 mL), H₂O (50 mL) was added. The mixture was extracted with DCM (50 mL×2). The combined organics were washed with brine (50 mL), dried over sodium sulfate, filtered and concentrated under vacuum. The resulting residue was purified by prep-TLC (eluting PE/EA=1/1) to afford (S)—N-(1-(1-(5-fluoropyridin-2-yl)ethyl)-1H-pyrrol-2-yl)-2-(hydroxymethyl)benzamide (319.18 mg, 74.81%) as a yellow oil. LC-MS: m/z 340.7 (M+H)⁺.

### Step D: 6-(cyclopropylmethyl)-4-{2,2-difluoro-4-[(5-fluoro-2-pyrimidinyl)methyl]-3-oxo-3,4-dihydro-2H-1,4-benzoxazin-7-yl}-1-[1-(5-fluoro-2-pyridyl)ethyl]-1,7-diaza-1H-indene-5-carboxamide (Compound 296)

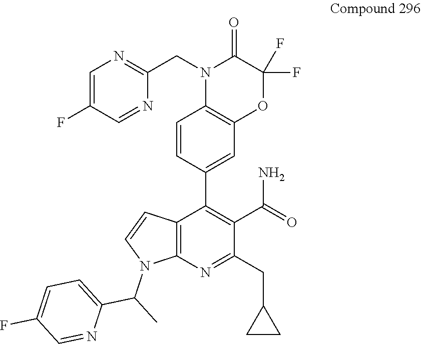 [SMILES: CC(c1ccc(F)cn1)n1ccc2c(-c3ccc4c(c3)OC(F)(F)C(=O)N4Cc3ncc(F)cn3)c(C(N)=O)c(CC3CC3)nc21]

To a solution of(S)—N-(1-(1-(5-fluoropyridin-2-yl)ethyl)-1H-pyrrol-2-yl)-2-(hydroxymethyl)benzamide (319.18 mg, 0.941 mmol) in acetic acid (6 mL) were added 2,2-difluoro-4-[(5-fluoropyrimidin-2-yl)methyl]-3-oxo-3,4-dihydro-2H-benzo[2,1-b][1,4]oxazine-7-carbaldehyde (304 mg, 0.941 mmol), 2-methylpropan-2-yl [(4-cyclopropyl-1,3-dioxobutyl)amino]methanoate (226.93 mg, 0.941 mmol) and ammonium acetate (72.49 mg, 0.941 mmol). The reaction mixture was stirred at 100° C. for 16 h in a sealed tube under N₂. The mixture was concentrated under vacuum. The resulting residue was purified by prep-TLC (eluting PE/EA=1/4) and prep-HPLC (Waters 3767/Qda Column: SunFire Sunfire C18, 19*250 mm*10 μm; Mobile Phase A: 0.1% FA/H₂O, B: ACN; Flow rate: 20 mL/min; Gradient: 61%-71%; Retention Time: 8.4 min of 16 min) to afford 6-(cyclopropylmethyl)-4-{2,2-difluoro-4-[(5-fluoro-2-pyrimidinyl)methyl]-3-oxo-3,4-dihydro-2H-1,4-benzoxazin-7-yl}-1-[1-(5-fluoro-2-pyridyl)ethyl]-1,7-diaza-1H-indene-5-carboxamide (2.74 mg, 0.43%).

¹H NMR (400 MHz, DMSO-d₆) δ 8.93 (s, 2H), 8.52 (d, J=3.2 Hz, 1H), 7.72 (d, J=3.6 Hz, 1H), 7.65-7.70 (m, 2H), 7.52 (s, 1H), 7.42 (d, J=1.6 Hz, 1H), 7.38 (s, 2H), 7.35 (dd, J₁=4.4 Hz, J₂=8.8 Hz, 1H), 6.21-6.26 (m, 2H), 5.50 (s, 2H), 2.65-2.78 (m, 2H), 1.91 (d, J=7.2 Hz, 3H), 1.19-1.23 (m, 1H), 0.36-0.40 (m, 2H), 0.22-0.24 (m, 2H). ¹⁹F NMR (376 MHz, DMSO-d₆) δ −74.96, −129.33, −139.31. LC-MS: m/z 632.3 (M+H)⁺.

### Example 63

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-(5-fluoro-2-pyrimidinyl)ethyl]-3-(1-hydroxy-1-methylethyl)-1,2,7-triaza-1H-indene-5-carboxamide (Compound 343)

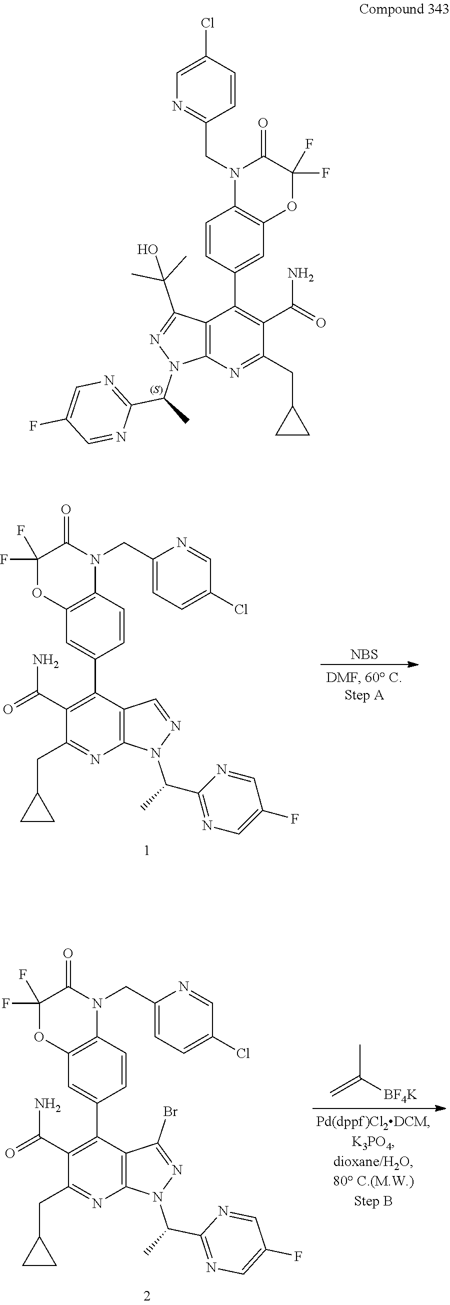

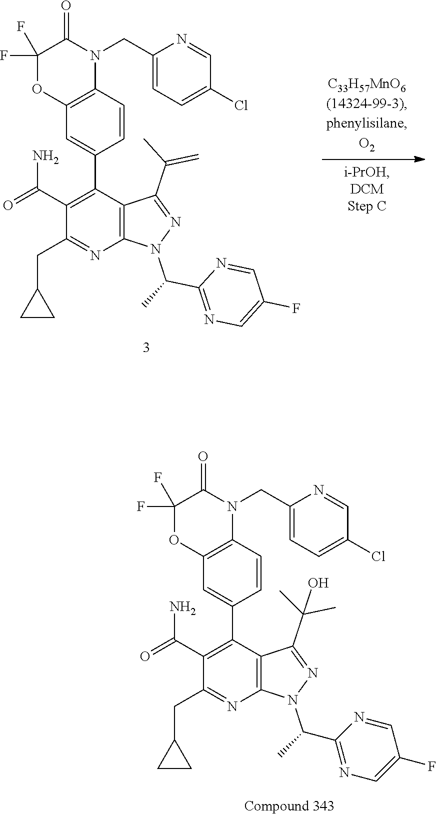 [SMILES: C=C(C)c1nn([C@@H](C)c2ncc(F)cn2)c2nc(CC3CC3)c(C(N)=O)c(-c3ccc4c(c3)OC(F)(F)C(=O)N4Cc3ccc(Cl)cn3)c12 ; C[C@@H](c1ncc(F)cn1)n1nc(C(C)(C)O)c2c(-c3ccc4c(c3)OC(F)(F)C(=O)N4Cc3ccc(Cl)cn3)c(C(N)=O)c(CC3CC3)nc21]

### Step A: 3-bromo-4-[4-[(5-chloro-2-pyridyl)methyl]-2,2-difluoro-3-oxo-1,4-benzoxazin-7-yl]-6-(cyclopropylmethyl)-1-[(1S)-1-(5-fluoropyrimidin-2-yl)ethyl]pyrazolo[3,4-b]pyridine-5-carboxamide

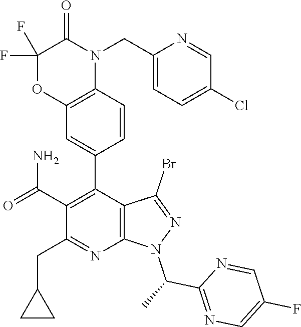 [SMILES: C[C@@H](c1ncc(F)cn1)n1nc(Br)c2c(-c3ccc4c(c3)OC(F)(F)C(=O)N4Cc3ccc(Cl)cn3)c(C(N)=O)c(CC3CC3)nc21]

To a solution of 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-(5-fluoro-2-pyrimidinyl)ethyl]-1,2,7-triaza-1H-indene-5-carboxamide (260.0 mg, 400.61 μmol) in DMF (3.0 mL) was added NBS (178.24 mg, 1.00 mmol) and the mixture was stirred under N₂ at 60° C. for 17 hours. The reaction mixture was diluted with H₂O (7 mL) and extracted with EtOAc (5 mL×3). The combined organic layer was concentrated under reduced pressure to give a crude. The crude was purified by chromatography column on silica gel eluted with 10~80% EtOAc in hexane to afford 3-bromo-4-[4-[(5-chloro-2-pyridyl)methyl]-2,2-difluoro-3-oxo-1,4-benzoxazin-7-yl]-6-(cyclopropylmethyl)-1-[(1S)-1-(5-fluoropyrimidin-2-yl)ethyl]pyrazolo[3,4-b]pyridine-5-carboxamide (215.0 mg, 73.73) as a yellow solid. LC-MS: m/z 727.0/729.0 (M+H)⁺.

### Step B: 4-[4-[(5-chloro-2-pyridyl)methyl]-2,2-difluoro-3-oxo-1,4-benzoxazin-7-yl]-6-(cyclopropylmethyl)-1-[(1S)-1-(5-fluoropyrimidin-2-yl)ethyl]-3-isopropenyl-pyrazolo[3,4-b]pyridine-5-carboxamide

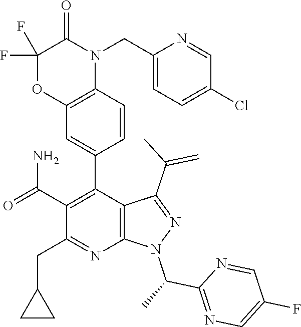 [SMILES: C=C(C)c1nn([C@@H](C)c2ncc(F)cn2)c2nc(CC3CC3)c(C(N)=O)c(-c3ccc4c(c3)OC(F)(F)C(=O)N4Cc3ccc(Cl)cn3)c12]

To a solution of 3-bromo-4-[4-[(5-chloro-2-pyridyl)methyl]-2,2-difluoro-3-oxo-1,4-benzoxazin-7-yl]-6-(cyclopropylmethyl)-1-[(1S)-1-(5-fluoropyrimidin-2-yl)ethyl]pyrazolo[3,4-b]pyridine-5-carboxamide (50.0 mg, 68.69 μmol), K₃PO₄ (43.74 mg, 206.07 μmol) and tetrafluoro (prop-1-en-2-yl)-15-borane potassium salt (30.49 mg, 206.07 μmol) in dioxane (2.0 mL) and H₂O (0.4 mL) was added Pd(dppf)Cl₂·DCM (11.22 mg, 13.74 μmol) under N₂ and the mixture was stirred at 80° C. (microwave) under N₂ for 0.5 hours. The volatiles were removed in vacuo to give a crude. The crude was purified by flash silica gel chromatography (ISCO®; 4 g SepaFlash® Silica Flash Column, Eluent of 0~60% Commercial hexanes: Ethyl acetate gradient @ 12 mL/min) to afford 4-[4-[(5-chloro-2-pyridyl)methyl]-2,2-difluoro-3-oxo-1,4-benzoxazin-7-yl]-6-(cyclopropylmethyl)-1-[(1S)-1-(5-fluoropyrimidin-2-yl)ethyl]-3-isopropenyl-pyrazolo[3,4-b]pyridine-5-carboxamide (70.0 mg, 94.84%). LC-MS: m/z 689.2 (M+H)⁺.

### Step C: 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-(5-fluoro-2-pyrimidinyl)ethyl]-3-(1-hydroxy-1-methylethyl)-1,2,7-triaza-1H-indene-5-carboxamide (Compound 343)

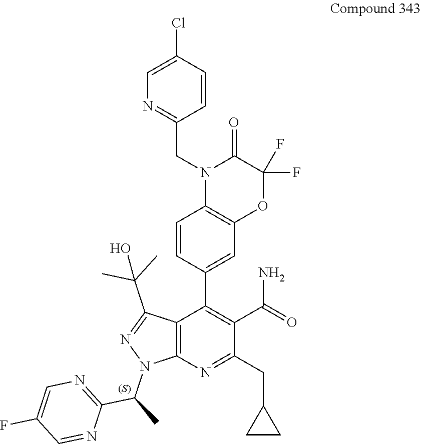

A mixture of 4-[4-[(5-chloro-2-pyridyl)methyl]-2,2-difluoro-3-oxo-1,4-benzoxazin-7-yl]-6-(cyclopropylmethyl)-1-[(1S)-1-(5-fluoropyrimidin-2-yl)ethyl]-3-isopropenyl-pyrazolo[3,4-b]pyridine-5-carboxamide (50.0 mg, 72.56 μmol), phenylsilane (15.70 mg, 145.12 μmol, 17.91 μL) and tris(((Z)-2,2,6,6-tetramethyl-5-oxohept-3-en-3-yl)oxy)manganese (4.39 mg, 7.26 μmol) in DCM (0.5 mL) and i-PrOH (5.0 mL) was degassed and purged with O₂ for 3 times, and then the mixture was stirred at 0° C. for 1 hour under 02 (15 Psi) atmosphere. The volatiles were removed in vacuo to give a crude. The crude was purified by prep-HPLC (column: 52-Welch Xtimate C18 150×30 mm, 5 μm; mobile phase: [H₂O (10 mM NH₄HCO₃)-ACN]; gradient: 60%-90% B over 7.0 min) and lyophilized to afford 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-(5-fluoro-2-pyrimidinyl)ethyl]-3-(1-hydroxy-1-methylethyl)-1,2,7-triaza-1H-indene-5-carboxamide (9.66 mg, 18.41%).

¹H NMR (400 MHz, DMSO-d₆) δ 8.89-8.83 (m, 2H), 8.58 (d, J=2.4 Hz, 1H), 7.99 (dd, J=8.4, 2.4 Hz, 1H), 7.69-7.61 (m, 1H), 7.55 (d, J=8.4 Hz, 1H), 7.41-7.30 (m, 2H), 7.29-7.24 (m, 1H), 7.23-7.13 (m, 1H), 6.36-6.23 (m, 1H), 5.53-5.41 (m, 1H), 5.40-5.30 (m, 1H), 4.47-4.35 (m, 1H), 2.70-2.57 (m, 2H), 2.03-1.93 (m, 3H), 1.32-1.20 (m, 3H), 1.11-1.07 (m, 1H), 1.07-1.01 (m, 3H), 0.39-0.25 (m, 2H), 0.19-0.07 (m, 2H). ¹⁹F NMR (376 MHz, DMSO-d₆) δ −72.72, −77.01, −140.37. LC-MS: m/z 707.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-(5-fluoro-2-pyrimidinyl)ethyl]-3-(1-hydroxyethyl)-1,2,7-triaza-1H-indene-5-carboxamide (Compound 344)

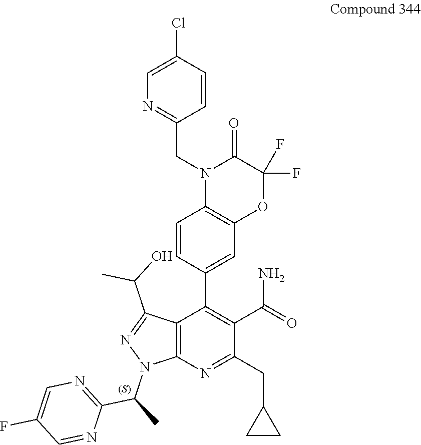

Compound 344 was synthesized following the similar route of Example 63, using tetrafluoro (vinyl)-15-borane potassium salt in step B.

¹H NMR (400 MHz, CDCl₃) δ 8.59-8.55 (m, 1H), 8.55-8.50 (m, 2H), 7.74-7.67 (m, 1H), 7.47-7.39 (m, 1H), 7.38-7.33 (m, 2H), 7.33-7.28 (m, 1H), 6.52-6.37 (m, 1H), 5.62-5.49 (m, 1H), 5.46-5.22 (m, 3H), 4.67-4.48 (m, 1H), 2.94-2.76 (m, 2H), 2.13 (d, J=7.2 Hz, 3H), 1.25-1.19 (m, 1H), 1.18-1.04 (m, 3H), 0.52-0.39 (m, 2H), 0.29-0.17 (m, 2H). ¹⁹F NMR (376 MHz, CDCl₃) δ −75.7, −79.01, −13.77. LC-MS: m/z 693.1 (M+H)⁺.

The following compounds were synthesized following the similar route of Previous Examples with appropriate starting materials.

### 6-(cyclopropylmethyl)-4-{2,2-difluoro-4-[1-(5-fluoro-2-pyrimidinyl)-2-methylpropyl]-3-oxo-3,4-dihydro-2H-1,4-benzoxazin-7-yl}-1-[1-(5-fluoro-2-pyridyl)cyclopropyl]-1,2,7-triaza-1H-indene-5-carboxamide (Compound 300)

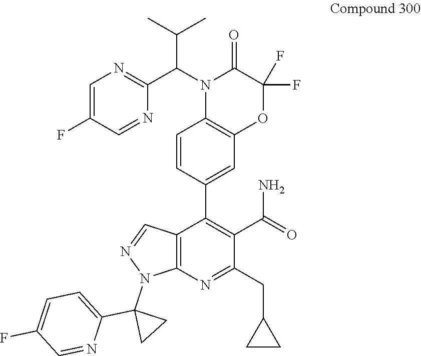 [SMILES: CC(C)C(c1ncc(F)cn1)N1C(=O)C(F)(F)Oc2cc(-c3c(C(N)=O)c(CC4CC4)nc4c3cnn4C3(c4ccc(F)cn4)CC3)ccc21]

¹H NMR (400 MHz, CDCl₃) δ 8.64 (s, 2H), 8.37 (d, J=2.8 Hz, 1H), 7.96 (s, 1H), 7.54-7.48 (m, 2H), 7.38 (dd, J=8.4, 1.6 Hz, 1H), 7.15-7.08 (m, 1H), 6.34-6.27 (m, 1H), 6.03-5.85 (m, 1H), 5.69-5.58 (m, 1H), 5.48-5.38 (m, 1H), 3.38-3.18 (m, 1H), 2.90 (d, J=6.8 Hz, 2H), 2.11-2.05 (m, 2H), 1.93-1.88 (m, 2H), 1.30-1.21 (m, 4H), 1.02 (d, J=6.8 Hz, 3H), 0.48-0.41 (m, 2H), 0.31-0.25 (m, 2H). ¹⁹F NMR (376 MHz, CDCl₃) δ −78.20, −131.53, −137.51. LC-MS: m/z 687.2 (M+H)⁺.

### 6-(cyclopropylmethyl)-4-{2,2-difluoro-4-[1-(5-fluoro-2-pyrimidinyl)-2-hydroxyethyl]-3-oxo-3,4-dihydro-2H-1,4-benzoxazin-7-yl}-1-[1-(5-fluoro-2-pyridyl)cyclopropyl]-1,2,7-triaza-1H-indene-5-carboxamide (Compound 301)

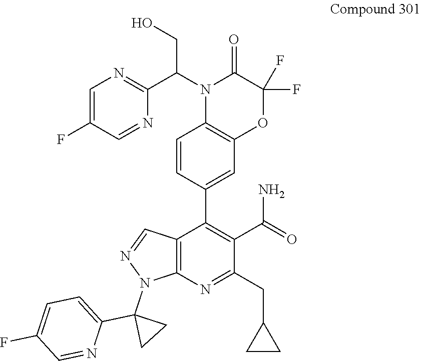 [SMILES: NC(=O)c1c(CC2CC2)nc2c(cnn2C2(c3ccc(F)cn3)CC2)c1-c1ccc2c(c1)OC(F)(F)C(=O)N2C(CO)c1ncc(F)cn1]

¹H NMR (400 MHz, CDCl₃) δ 8.64 (s, 2H), 8.37 (d, J=2.8 Hz, 1H), 8.00 (s, 1H), 7.60 (d, J=2.0 Hz, 1H), 7.51 (dd, J=8.4, 2.0 Hz, 1H), 7.31-7.28 (m, 1H), 7.17-7.10 (m, 1H), 6.38-6.31 (m, 1H), 5.86-5.78 (m, 1H), 5.78-5.72 (m, 1H), 5.53-5.48 (m, 1H), 4.89-4.81 (m, 1H), 4.31-4.22 (m, 1H), 2.91 (d, J=7.2 Hz, 2H), 2.11-2.06 (m, 2H), 1.93-1.90 (m, 2H), 1.29-1.22 (m, 1H), 0.49-0.42 (m, 2H), 0.31-0.26 (m, 2H). ¹⁹F NMR (376 MHz, CDCl₃) δ −77.40, −79.56, −131.44, −137.20. LC-MS: m/z 675.2 (M+H)⁺.

### 7-[5-carbamoyl-6-(cyclopropylmethyl)-1-[1-(5-fluoro-2-pyridyl)cyclopropyl]-1,2,7-triaza-1H-inden-4-yl]-4-[(5-fluoro-2-pyrimidinyl)methyl]-3-oxo-3,4-dihydro-2H-1,4-benzoxazin-2-yl acetate (Compound 304)

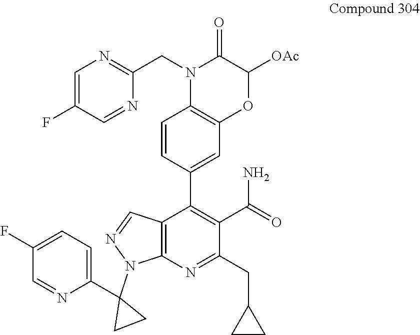 [SMILES: CC(=O)OC1Oc2cc(-c3c(C(N)=O)c(CC4CC4)nc4c3cnn4C3(c4ccc(F)cn4)CC3)ccc2N(Cc2ncc(F)cn2)C1=O]

¹H NMR (400 MHz, CDCl₃) δ 8.62 (s, 2H), 8.37 (d, J=2.8 Hz, 1H), 7.96 (s, 1H), 7.45 (d, J=1.6 Hz, 1H), 7.37 (dd, J=8.0, 1.6 Hz, 1H), 7.15-7.06 (m, 2H), 6.86 (s, 1H), 6.34-6.27 (m, 1H), 5.65-5.61 (m, 1H), 5.60-5.54 (m, 1H), 5.49-5.43 (m, 1H), 5.42-5.33 (m, 1H), 2.90 (d, J=6.8 Hz, 2H), 2.15 (s, 3H), 2.10-2.04 (m, 2H), 1.95-1.86 (m, 2H), 1.25-1.18 (m, 1H), 0.49-0.39 (m, 2H), 0.33-0.22 (m, 2H). ¹⁹F NMR (376 MHz, CDCl₃) δ −131.56, −138.29. LC-MS: m/z 667.1 (M−H)⁺.

### 4-{4-[(5-chloro-2-pyrimidinyl)methyl]-2-hydroxy-2-methyl-3-oxo-3,4-dihydro-2H-1,4-benzoxazin-7-yl}-6-(cyclopropylmethyl)-1-[1-(5-fluoro-2-pyridyl)cyclopropyl]-1,2,7-triaza-1H-indene-5-carboxamide (Compound 305)

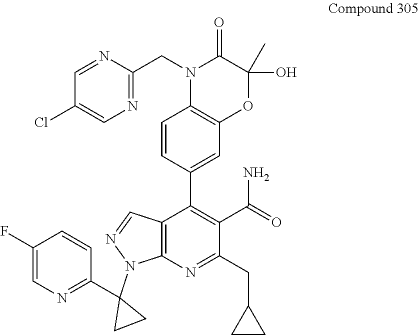 [SMILES: CC1(O)Oc2cc(-c3c(C(N)=O)c(CC4CC4)nc4c3cnn4C3(c4ccc(F)cn4)CC3)ccc2N(Cc2ncc(Cl)cn2)C1=O]

¹H NMR (400 MHz, CDCl₃) δ 8.70 (s, 2H), 8.37 (d, J=2.8 Hz, 1H), 8.01 (s, 1H), 7.45 (d, J=1.6 Hz, 1H), 7.30 (dd, J=8.8, 2.0 Hz, 1H), 7.15-7.09 (m, 1H), 6.93 (d, J=8.4 Hz, 1H), 6.34-6.29 (m, 1H), 5.74-5.66 (m, 2H), 5.48-5.42 (m, 1H), 5.21-5.13 (m, 1H), 4.67-4.59 (m, 1H), 2.90 (d, J=6.8 Hz, 2H), 2.12-2.04 (m, 2H), 1.95-1.89 (m, 2H), 1.88 (s, 3H), 1.31-1.24 (m, 1H), 0.50-0.38 (m, 2H), 0.32-0.22 (m, 2H). ¹⁹F NMR (376 MHz, CDCl₃) δ −131.61. LC-MS: m/z 655.2 (M+H)⁺.

### 4-{4-[(5-chloro-2-pyrimidinyl)methyl]-2-ethoxy-2-methyl-3-oxo-3,4-dihydro-2H-1,4-benzoxazin-7-yl}-6-(cyclopropylmethyl)-1-[1-(5-fluoro-2-pyridyl)cyclopropyl]-1,2,7-triaza-1H-indene-5-carboxamide (Compound 306)

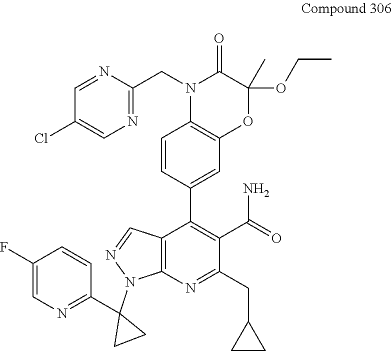 [SMILES: CCOC1(C)Oc2cc(-c3c(C(N)=O)c(CC4CC4)nc4c3cnn4C3(c4ccc(F)cn4)CC3)ccc2N(Cc2ncc(Cl)cn2)C1=O]

¹H NMR (400 MHz, CDCl₃) δ 8.70 (s, 2H), 8.37 (d, J=2.8 Hz, 1H), 7.98 (s, 1H), 7.41 (d, J=1.6 Hz, 1H), 7.29 (dd, J=8.4, 2.0 Hz, 1H), 7.15-7.08 (m, 1H), 7.00 (d, J=8.4 Hz, 1H), 6.35-6.30 (m, 1H), 5.59-5.54 (m, 1H), 5.53-5.45 (m, 1H), 5.41-5.32 (m, 2H), 3.83-3.69 (m, 2H), 2.89 (d, J=6.8 Hz, 2H), 2.12-2.05 (m, 2H), 1.93-1.89 (m, 2H), 1.88 (s, 3H), 1.27-1.25 (m, 1H), 1.11 (t, J=6.8 Hz, 3H), 0.49-0.41 (m, 2H), 0.32-0.25 (m, 2H). ¹⁹F NMR (376 MHz, CDCl₃) δ −131.62. LC-MS: m/z 683.2 (M+H)⁺.

### 4-{1-[(5-chloro-2-pyrimidinyl)methyl]-3-methyl-2-oxo-1,2-dihydro-6-quinoxalinyl}-6-(cyclopropylmethyl)-1-[1-(5-fluoro-2-pyridyl)cyclopropyl]-1,2,7-triaza-1H-indene-5-carboxamide (Compound 307)

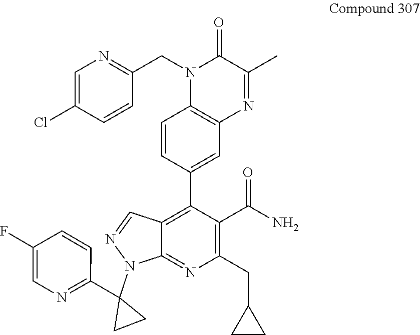 [SMILES: Cc1nc2cc(-c3c(C(N)=O)c(CC4CC4)nc4c3cnn4C3(c4ccc(F)cn4)CC3)ccc2n(Cc2ccc(Cl)cn2)c1=O]

Compound 307 was synthesized following the similar route of Example 56, using 1-((5-chloropyrimidin-2-yl)methyl)-3-methyl-2-oxo-1,2-dihydroquinoxaline-6-carbaldehyde in step A.

¹H NMR (400 MHz, DMSO-d₆) δ 8.93 (s, 2H), 8.52 (d, J=2.8 Hz, 1H), 8.12 (d, J=2.0 Hz, 1H), 8.08 (s, 1H), 7.90 (s, 1H), 7.76-7.79 (m, 1H), 7.50-7.61 (m, 3H), 6.36 (q, J=4.4 Hz, 1H), 5.74 (s, 2H), 2.77 (d, J=6.8 Hz, 2H), 2.51 (s, 3H), 1.94-1.97 (m, 2H), 1.82-1.85 (m, 2H), 1.15-1.23 (m, 1H), 0.36-0.40 (m, 2H), 0.21-0.26 (m, 2H). ¹⁹F NMR (376 MHz, DMSO-d₆) δ −131.52. LC-MS: m/z 636.3 (M+H)⁺.

### 4-{4-[(5-chloro-2-pyridyl)methyl]-2-methyl-3-oxo-1,4,5-triaza-3,4-dihydro-7-naphthyl}-6-(cyclopropylmethyl)-1-[1-(5-fluoro-2-pyridyl)cyclopropyl]-1,2,7-triaza-1H-indene-5-carboxamide (Compound 308)

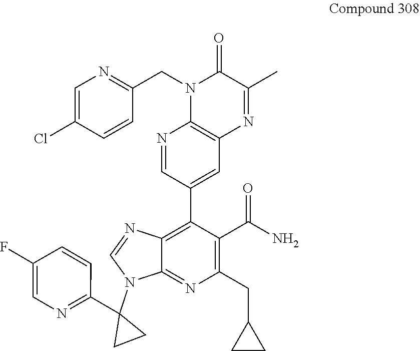 [SMILES: Cc1nc2cc(-c3c(C(N)=O)c(CC4CC4)nc4c3ncn4C3(c4ccc(F)cn4)CC3)cnc2n(Cc2ccc(Cl)cn2)c1=O]

Compound 308 was synthesized following the similar route of Example 56, using 4-((5-chloropyridin-2-yl)methyl)-2-methyl-3-oxo-3,4-dihydropyrido[2,3-b]pyrazine-7-carbaldehyde in step A.

¹H NMR (400 MHz, DMSO-d₆) δ 8.74 (d, J=2.0 Hz, 1H), 8.47-8.52 (m, 3H), 8.16 (s, 1H), 7.96 (d, J=2.0 Hz, 1H), 7.91 (dd, J₁=2.4 Hz, J₂=8.4 Hz, 1H), 7.65 (s, 1H), 7.49-7.55 (m, 2H), 6.36-6.39 (m, 1H), 5.73 (s, 2H), 2.78 (d, J=6.8 Hz, 2H), 2.54 (s, 3H), 1.94-1.98 (m, 2H), 1.82-1.85 (m, 2H), 1.15-1.23 (m, 1H), 0.36-0.40 (m, 2H), 0.21-0.26 (m, 2H). ¹⁹F NMR (376 MHz, DMSO-d₆) δ −131.46. LC-MS: m/z 636.4 (M+H)⁺.

### 4-{2,2-difluoro-4-[(5-fluoro-2-pyrimidinyl)methyl]-3-oxo-3,4-dihydro-2H-1,4-benzoxazin-7-yl}-1-[1-(5-fluoro-2-pyridyl)cyclopropyl]-6-(2-hydroxypropyl)-1,2,7-triaza-1H-indene-5-carboxamide (Compound 309)

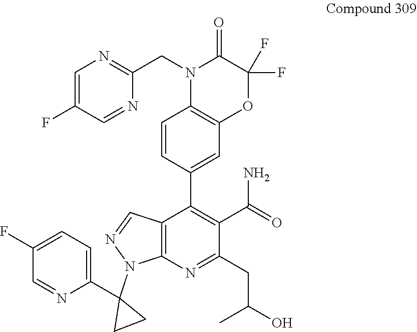 [SMILES: CC(O)Cc1nc2c(cnn2C2(c3ccc(F)cn3)CC2)c(-c2ccc3c(c2)OC(F)(F)C(=O)N3Cc2ncc(F)cn2)c1C(N)=O]

¹H NMR (400 MHz, DMSO-d₆) δ 8.95 (s, 2H), 8.51 (d, J=2.8 Hz, 1H), 8.09 (s, 1H), 7.92-7.87 (m, 1H), 7.73-7.69 (m, 1H), 7.67 (d, J=2.0 Hz, 1H), 7.55-7.48 (m, 2H), 7.46 (d, J=8.4 Hz, 1H), 6.36-6.29 (m, 1H), 5.53 (s, 2H), 4.78-4.64 (m, 1H), 4.25-4.09 (m, 1H), 3.08-2.99 (m, 1H), 2.92-2.84 (m, 1H), 1.97-1.91 (m, 2H), 1.85-1.79 (m, 2H), 1.04 (d, J=6.0 Hz, 3H). ¹⁹F NMR (376 MHz, DMSO-d₆) δ −74.91, −131.45, −139.26. LC-MS: m/z 649.2 (M+H)⁺.

### 3-chloro-6-(cyclopropylmethyl)-4-{2,2-difluoro-4-[(5-fluoro-2-pyrimidinyl)methyl]-3-oxo-3,4-dihydro-2H-1,4-benzoxazin-7-yl}-1-[1-(5-fluoro-2-pyridyl)-2-hydroxyethyl]-1,2,7-triaza-1H-indene-5-carboxamide (Compound 310)

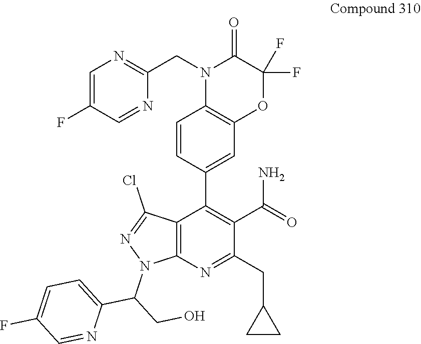 [SMILES: NC(=O)c1c(CC2CC2)nc2c(c(Cl)nn2C(CO)c2ccc(F)cn2)c1-c1ccc2c(c1)OC(F)(F)C(=O)N2Cc1ncc(F)cn1]

¹H NMR (400 MHz, CDCl₃) δ 8.60 (s, 2H), 8.43 (d, J=2.4 Hz, 1H), 7.38 (d, J=1.6 Hz, 1H), 7.36-7.30 (m, 1H), 7.27-7.20 (m, J=8.4 Hz, 1H), 7.03 (d, J=8.4 Hz, 2H), 6.29-6.22 (m, 1H), 5.75-5.59 (m, 1H), 5.59-5.34 (m, 3H), 4.66-4.46 (m, 2H), 2.95-2.82 (m, 2H), 1.30-1.17 (m, 1H), 0.60-0.44 (m, 2H), 0.36-0.19 (m, 2H). ¹⁹F NMR (376 MHz, CDCl₃) δ −75.90, −77.96, −127.74, −137.84. LC-MS: m/z 683.1 (M+H)⁺.

### 6-(cyclopropylmethyl)-4-{2,2-difluoro-4-[(5-fluoro-2-pyrimidinyl)methyl]-3-oxo-3,4-dihydro-2H-1,4-benzoxazin-7-yl}-1-[1-(5-fluoro-2-pyridyl)-2-hydroxyethyl]-1,2,7-triaza-1H-indene-5-carboxamide (Compound 311)

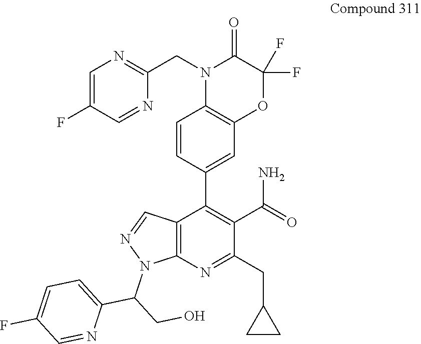 [SMILES: NC(=O)c1c(CC2CC2)nc2c(cnn2C(CO)c2ccc(F)cn2)c1-c1ccc2c(c1)OC(F)(F)C(=O)N2Cc1ncc(F)cn1]

¹H NMR (400 MHz, CDCl₃) δ 8.61 (s, 2H), 8.44 (d, J=2.4 Hz, 1H), 7.95 (s, 1H), 7.55 (d, J=1.6 Hz, 1H), 7.43 (dd, J=8.0, 2.0 Hz, 1H), 7.34-7.28 (m, 1H), 7.08 (d, J=8.4 Hz, 1H), 6.94-6.89 (m, 1H), 6.27 (t, J=4.8 Hz, 1H), 5.74-5.67 (m, 1H), 5.54-5.45 (m, 3H), 4.80-4.66 (m, 1H), 4.61-4.55 (m, 2H), 2.96-2.90 (m, 2H), 1.31-1.21 (m, 1H), 0.54-0.48 (m, 2H), 0.30-0.24 (m, 2H). ¹⁹F NMR (376 MHz, CDCl₃) δ −76.75, −128.22, −137.80. LC-MS: m/z 649.2 (M+H)⁺.

### 1-[(1,3,3a-triaza-4,5,6,7-tetrahydro-6-indenyl)methyl]-3-chloro-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-indene-5-carboxamide (Compound 312)

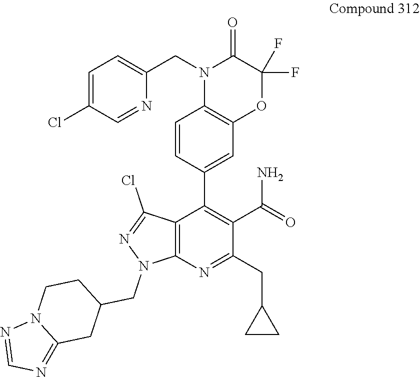 [SMILES: NC(=O)c1c(CC2CC2)nc2c(c(Cl)nn2CC2CCn3ncnc3C2)c1-c1ccc2c(c1)OC(F)(F)C(=O)N2Cc1ccc(Cl)cn1]

¹H NMR (400 MHz, CDCl₃) δ 8.57 (d, J=2.0 Hz, 1H), 7.85 (s, 1H), 7.71 (dd, J=8.4, 2.4 Hz, 1H), 7.43 (d, J=8.4 Hz, 1H), 7.38-7.34 (m, 2H), 7.31-7.28 (m, 1H), 5.57 (br s, 1H), 5.46-5.38 (m, 2H), 5.35-5.26 (m, 1H), 4.66-4.60 (m, 2H), 4.44-4.37 (m, 1H), 4.13-4.06 (m, 1H), 3.01-2.91 (m, 3H), 2.82-2.66 (m, 2H), 2.26-2.19 (m, 1H), 1.98-1.89 (m, 1H), 1.32-1.28 (m, 1H), 0.58-0.52 (m, 2H), 0.34-0.28 (m, 2H). LC-MS: m/z 694.2 (M−H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-{[1-(N,N-dimethylcarbamoyl)-4-piperidyl]methyl}-1,2,7-triaza-1H-indene-5-carboxamide (Compound 313)

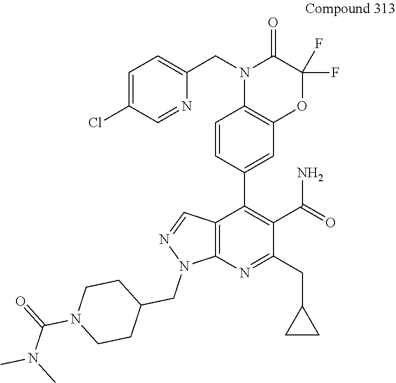 [SMILES: CN(C)C(=O)N1CCC(Cn2ncc3c(-c4ccc5c(c4)OC(F)(F)C(=O)N5Cc4ccc(Cl)cn4)c(C(N)=O)c(CC4CC4)nc32)CC1]

¹H NMR (400 MHz, DMSO-d₆) δ 8.59 (d, J=2.4 Hz, 1H), 7.99 (dd, J=8.4, 2.4 Hz, 1H), 7.94 (s, 1H), 7.91-7.86 (m, 1H), 7.64-7.57 (m, 3H), 7.50 (dd, J=8.4, 1.6 Hz, 1H), 7.44 (d, J=8.8 Hz, 1H), 5.43 (s, 2H), 4.40 (d, J=6.8 Hz, 2H), 3.56-3.42 (m, 2H), 2.80 (d, J=6.8 Hz, 2H), 2.69 (s, 6H), 2.66-2.55 (m, 2H), 2.22-2.08 (m, 1H), 1.52-1.41 (m, 2H), 1.35-1.14 (m, 3H), 0.49-0.42 (m, 2H), 0.33-0.27 (m, 2H). ¹⁹F NMR (376 MHz, DMSO-d₆) δ −74.46. LC-MS: m/z 693.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-{[1-(N-methylcarbamoyl)-4-piperidyl]methyl}-1,2,7-triaza-1H-indene-5-carboxamide (Compound 314)

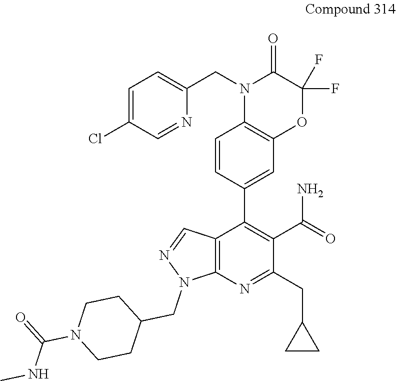 [SMILES: CNC(=O)N1CCC(Cn2ncc3c(-c4ccc5c(c4)OC(F)(F)C(=O)N5Cc4ccc(Cl)cn4)c(C(N)=O)c(CC4CC4)nc32)CC1]

¹H NMR (400 MHz, DMSO-d₆) δ 8.59 (d, J=2.4 Hz, 1H), 8.00 (dd, J=8.0, 2.4 Hz, 1H), 7.93 (s, 1H), 7.91-7.86 (m, 1H), 7.64-7.58 (m, 3H), 7.50 (dd, J=8.4, 1.6 Hz, 1H), 7.44 (d, J=8.8 Hz, 1H), 6.37-6.30 (m, 1H), 5.44 (s, 2H), 4.39 (d, J=6.4 Hz, 2H), 3.92-3.83 (m, 2H), 2.81 (d, J=6.8 Hz, 2H), 2.65-2.54 (m, 2H), 2.52 (d, J=4.4 Hz, 3H), 2.21-2.09 (m, 1H), 1.50-1.39 (m, 2H), 1.33-1.26 (m, 1H), 1.16-1.03 (m, 2H), 0.53-0.41 (m, 2H), 0.36-0.26 (m, 2H). ¹⁹F NMR (376 MHz, DMSO-d₆) δ −74.45. LC-MS: m/z 679.2 (M+H)⁺.

### 2,2-difluoroethyl 4-[(5-carbamoyl-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-inden-1-yl)methyl]-1-piperidinecarboxylate (Compound 315)

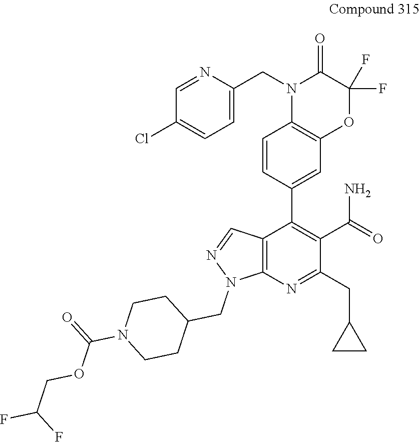 [SMILES: NC(=O)c1c(CC2CC2)nc2c(cnn2CC2CCN(C(=O)OCC(F)F)CC2)c1-c1ccc2c(c1)OC(F)(F)C(=O)N2Cc1ccc(Cl)cn1]

¹H NMR (400 MHz, DMSO-d₆) δ 8.60 (d, J=2.0 Hz, 1H), 8.02 (dd, J=8.4, 2.4 Hz, 1H), 7.95 (s, 1H), 7.91-7.85 (m, 1H), 7.65-7.57 (m, 3H), 7.49 (dd, J=8.4, 1.6 Hz, 1H), 7.44 (d, J=8.4 Hz, 1H), 6.38-6.06 (m, 1H), 5.43 (s, 2H), 4.40 (d, J=6.8 Hz, 2H), 4.32-4.20 (m, 2H), 4.02-3.88 (m, 2H), 2.92-2.73 (m, 4H), 2.28-2.11 (m, 1H), 1.59-1.46 (m, 2H), 1.32-1.11 (m, 3H), 0.53-0.40 (m, 2H), 0.36-0.22 (m, 2H). ¹⁹F NMR (376 MHz, DMSO-d₆) δ −74.45, −126.24. LC-MS: m/z 730.2 (M+H)⁺.

### p-fluorophenyl 4-[(5-carbamoyl-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-inden-1-yl)methyl]-1-piperidinecarboxylate (Compound 316)

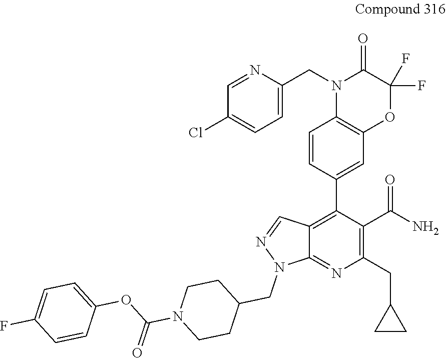 [SMILES: NC(=O)c1c(CC2CC2)nc2c(cnn2CC2CCN(C(=O)Oc3ccc(F)cc3)CC2)c1-c1ccc2c(c1)OC(F)(F)C(=O)N2Cc1ccc(Cl)cn1]

¹H NMR (400 MHz, CDCl₃) δ 8.57 (d, J=2.4 Hz, 1H), 7.87 (s, 1H), 7.71 (dd, J=8.4, 2.4 Hz, 1H), 7.53 (s, 1H), 7.50-7.44 (m, 2H), 7.37 (d, J=8.4 Hz, 1H), 7.09-6.98 (m, 4H), 5.68 (br s, 1H), 5.46 (br s, 1H), 5.35 (s, 2H), 4.51 (d, J=6.8 Hz, 2H), 4.36-4.14 (m, 2H), 2.96 (d, J=6.8 Hz, 3H), 2.90-2.72 (m, 1H), 2.42-2.27 (m, 1H), 1.66 (br s, 2H), 1.46-1.26 (m, 3H), 0.60-0.51 (m, 2H), 0.40-0.30 (m, 2H). ¹⁹F NMR (376 MHz, CDCl₃) δ −76.94, −117.97. LC-MS: m/z 760.3 (M+H)⁺.

### Benzyl 4-[(5-carbamoyl-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-inden-1-yl)methyl]-1-piperidinecarboxylate (Compound 317)

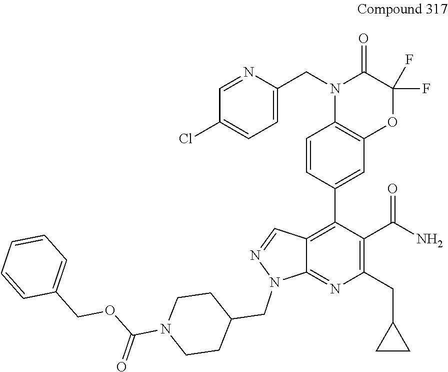 [SMILES: NC(=O)c1c(CC2CC2)nc2c(cnn2CC2CCN(C(=O)OCc3ccccc3)CC2)c1-c1ccc2c(c1)OC(F)(F)C(=O)N2Cc1ccc(Cl)cn1]

¹H NMR (400 MHz, DMSO-d₆) δ 8.59 (d, J=2.4 Hz, 1H), 7.99 (dd, J=8.4, 2.4 Hz, 1H), 7.93 (s, 1H), 7.85-7.79 (m, 1H), 7.63-7.57 (m, 2H), 7.56-7.53 (m, 1H), 7.49 (dd, J=8.4, 1.6 Hz, 1H), 7.44 (d, J=8.4 Hz, 1H), 7.38-7.27 (m, 5H), 5.44 (s, 2H), 5.04 (s, 2H), 4.38 (d, J=6.8 Hz, 2H), 4.06-3.90 (m, 2H), 2.87-2.72 (m, 4H), 2.27-2.13 (m, 1H), 1.59-1.47 (m, 2H), 1.35-1.25 (m, 1H), 1.24-1.10 (m, 2H), 0.54-0.40 (m, 2H), 0.35-0.25 (m, 2H). ¹⁹F NMR (376 MHz, DMSO-d₆) δ −74.51. LC-MS: m/z 756.2 (M+H)⁺.

### (p-fluorophenyl)methyl 4-[(5-carbamoyl-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-inden-1-yl)methyl]-1-piperidinecarboxylate (Compound 318)

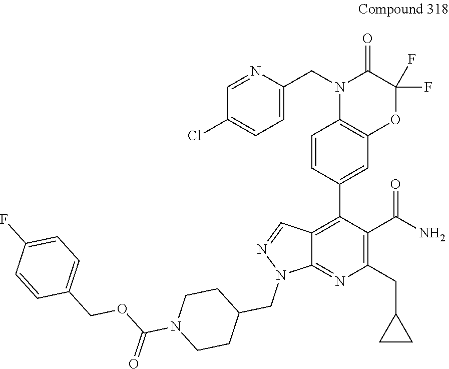 [SMILES: NC(=O)c1c(CC2CC2)nc2c(cnn2CC2CCN(C(=O)OCc3ccc(F)cc3)CC2)c1-c1ccc2c(c1)OC(F)(F)C(=O)N2Cc1ccc(Cl)cn1]

¹H NMR (400 MHz, CDCl₃) δ 8.57 (d, J=2.0 Hz, 1H), 7.84 (s, 1H), 7.71 (dd, J=2.4, 8.4 Hz, 1H), 7.52 (s, 1H), 7.48-7.45 (m, 2H), 7.38-7.30 (m, 3H), 7.04 (t, J=8.8 Hz, 2H), 5.63 (br s, 1H), 5.43 (br s, 1H), 5.35 (s, 2H), 5.07 (s, 2H), 4.46 (d, J=6.8 Hz, 2H), 4.27-4.05 (m, 2H), 2.94 (d, J=6.8 Hz, 2H), 2.85-2.68 (m, 2H), 2.37-2.18 (m, 1H), 1.55-1.51 (m, 1H), 1.47-1.19 (m, 4H), 0.56-0.49 (m, 2H), 0.34 (br d, J=4.4 Hz, 2H). ¹⁹F NMR (376 MHz, CDCl₃) δ −76.94, −114.12. LC-MS: m/z 774.4 (M+H)⁺.

### 1-methyl-3-pyrazolyl)methyl 4-[(5-carbamoyl-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-inden-1-yl)methyl]-1-piperidinecarboxylate (Compound 319)

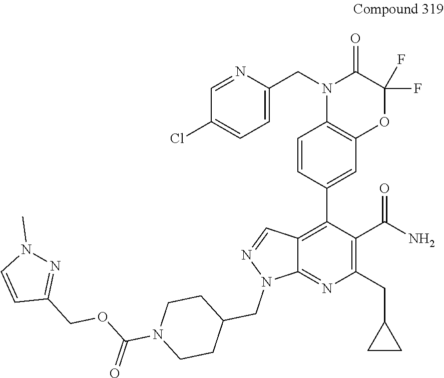 [SMILES: Cn1ccc(COC(=O)N2CCC(Cn3ncc4c(-c5ccc6c(c5)OC(F)(F)C(=O)N6Cc5ccc(Cl)cn5)c(C(N)=O)c(CC5CC5)nc43)CC2)n1]

¹H NMR (400 MHz, CDCl₃) δ 8.55 (d, J=2.4 Hz, 1H), 7.82 (s, 1H), 7.71 (dd, J=2.4, 8.4 Hz, 1H), 7.54-7.50 (m, 1H), 7.49-7.40 (m, 2H), 7.35 (d, J=8.4 Hz, 1H), 7.30 (d, J=2.0 Hz, 1H), 6.26 (d, J=2.4 Hz, 1H), 5.85-5.69 (m, 1H), 5.62-5.45 (m, 1H), 5.34 (s, 2H), 5.08 (s, 2H), 4.45 (d, J=6.8 Hz, 2H), 4.29-4.02 (m, 2H), 3.88 (s, 3H), 2.93 (d, J=7.2 Hz, 2H), 2.83-2.64 (m, 2H), 2.28-2.20 (m, 1H), 1.61-1.49 (m, 2H), 1.35-1.22 (m, 3H), 0.57-0.48 (m, 2H), 0.37-0.28 (m, 2H). ¹⁹F NMR (376 MHz, CDCl₃) δ −76.92. LC-MS: m/z 760.2 (M+H)⁺.

### 1-[(1-benzoyl-4-piperidyl)methyl]-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-indene-5-carboxamide (Compound 320)

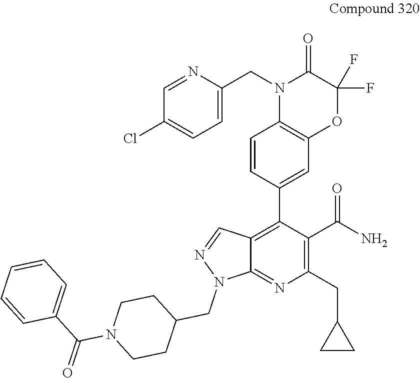 [SMILES: NC(=O)c1c(CC2CC2)nc2c(cnn2CC2CCN(C(=O)c3ccccc3)CC2)c1-c1ccc2c(c1)OC(F)(F)C(=O)N2Cc1ccc(Cl)cn1]

¹H NMR (400 MHz, CDCl₃) δ 8.56 (d, J=2.0 Hz, 1H), 7.85 (s, 1H), 7.71 (dd, J=8.0, 2.4 Hz, 1H), 7.53-7.50 (m, 1H), 7.49-7.42 (m, 2H), 7.42-7.31 (m, 6H), 5.68 (br.s., 1H), 5.54 (br.s., 1H), 5.34 (s, 2H), 4.81-4.59 (m, 1H), 4.50 (d, J=6.8 Hz, 2H), 3.90-3.63 (m, 1H), 2.94 (d, J=6.4 Hz, 3H), 2.86-2.69 (m, 1H), 2.46-2.29 (m, 1H), 1.62-1.24 (m, 5H), 0.60-0.45 (m, 2H), 0.39-0.27 (m, 2H). ¹⁹F NMR (376 MHz, CDCl₃) δ −76.93. LC-MS: m/z 726.1 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-{[1-(2-phenylacetyl)-4-piperidyl]methyl}-1,2,7-triaza-1H-indene-5-carboxamide (Compound 321)

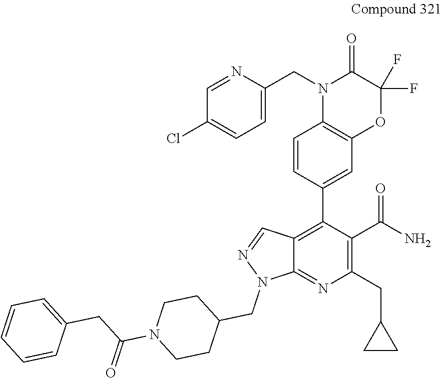 [SMILES: NC(=O)c1c(CC2CC2)nc2c(cnn2CC2CCN(C(=O)Cc3ccccc3)CC2)c1-c1ccc2c(c1)OC(F)(F)C(=O)N2Cc1ccc(Cl)cn1]

¹H NMR (400 MHz, CDCl₃) δ 8.57 (d, J=2.0 Hz, 1H), 7.83 (s, 1H), 7.71 (dd, J=8.4, 2.4 Hz, 1H), 7.51 (s, 1H), 7.49-7.43 (m, 2H), 7.36 (d, J=8.4 Hz, 1H), 7.34-7.28 (m, 2H), 7.26-7.19 (m, 3H), 5.71-5.58 (m, 1H), 5.53-5.41 (m, 1H), 5.35 (s, 2H), 4.68-4.54 (m, 1H), 4.42 (d, J=6.8 Hz, 2H), 3.94-3.80 (m, 1H), 3.72 (s, 2H), 3.01-2.86 (m, 3H), 2.68-2.51 (m, 1H), 2.37-2.19 (m, 1H), 1.53-1.48 (m, 2H), 1.35-1.16 (m, 2H), 1.15-0.98 (m, 1H), 0.56-0.48 (m, 2H), 0.38-0.30 (m, 2H). ¹⁹F NMR (376 MHz, CDCl₃) δ −76.94. LC-MS: m/z 740.1 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-{[1-(3-phenylpropionyl)-4-piperidyl]methyl}-1,2,7-triaza-1H-indene-5-carboxamide (Compound 322)

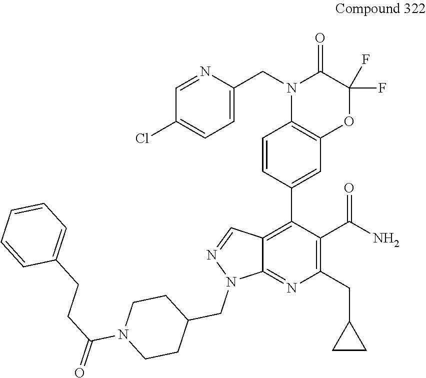 [SMILES: NC(=O)c1c(CC2CC2)nc2c(cnn2CC2CCN(C(=O)CCc3ccccc3)CC2)c1-c1ccc2c(c1)OC(F)(F)C(=O)N2Cc1ccc(Cl)cn1]

¹H NMR (400 MHz, DMSO-d₆) δ 8.59 (d, J=2.4 Hz, 1H), 8.00 (dd, J=8.4, 2.4 Hz, 1H), 7.93 (s, 1H), 7.87 (d, J=1.6 Hz, 1H), 7.64-7.57 (m, 3H), 7.49 (dd, J=8.4, 2.0 Hz, 1H), 7.45 (d, J=8.4 Hz, 1H), 7.28-7.19 (m, 4H), 7.18-7.13 (m, 1H), 5.43 (s, 2H), 4.47-4.26 (m, 3H), 3.87-3.77 (m, 1H), 2.94-2.86 (m, 1H), 2.84-2.75 (m, 4H), 2.59-2.54 (m, 3H), 2.28-2.15 (m, 1H), 1.53-1.42 (m, 2H), 1.33-1.24 (m, 1H), 1.14-0.97 (m, 2H), 0.48-0.43 (m, 2H), 0.33-0.27 (m, 2H). ¹⁹F NMR (376 MHz, DMSO-d₆) δ −74.47. LC-MS: m/z 754.3 (M+H)⁺.

### methyl 3-[(5-carbamoyl-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-inden-1-yl)methyl]-1-piperidinecarboxylate (Compound 323)

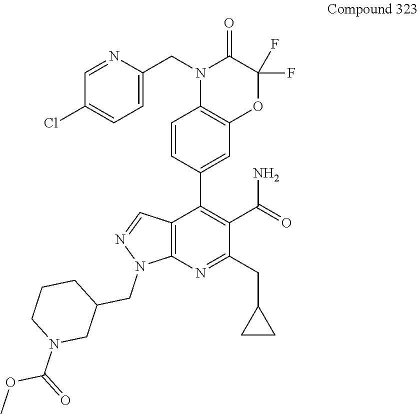 [SMILES: COC(=O)N1CCCC(Cn2ncc3c(-c4ccc5c(c4)OC(F)(F)C(=O)N5Cc4ccc(Cl)cn4)c(C(N)=O)c(CC4CC4)nc32)C1]

¹H NMR (400 MHz, DMSO-d₆) δ 8.59 (d, J=2.0 Hz, 1H), 8.01 (dd, J=8.4, 2.4 Hz, 1H), 7.96 (s, 1H), 7.93-7.87 (m, 1H), 7.66-7.57 (m, 3H), 7.50 (dd, J=8.8, 1.6 Hz, 1H), 7.45 (d, J=8.8 Hz, 1H), 5.42 (s, 2H), 4.39 (d, J=6.8 Hz, 2H), 3.83-3.71 (m, 2H), 3.52 (s, 3H), 2.97-2.65 (m, 4H), 2.21-2.08 (m, 1H), 1.71-1.58 (m, 2H), 1.30-1.18 (m, 3H), 0.53-0.40 (m, 2H), 0.37-0.23 (m, 2H). ¹⁹F NMR (376 MHz, DMSO-d₆) δ −74.46. LC-MS: m/z 680.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(4-methoxycyclohexyl)methyl]-1,2,7-triaza-1H-indene-5-carboxamide (Compound 324)

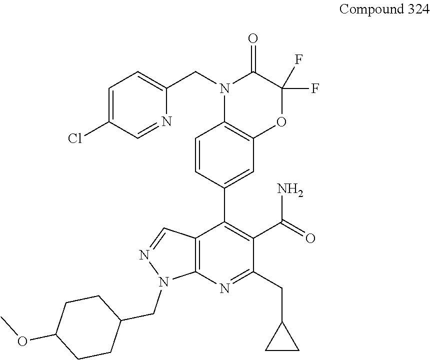 [SMILES: COC1CCC(Cn2ncc3c(-c4ccc5c(c4)OC(F)(F)C(=O)N5Cc4ccc(Cl)cn4)c(C(N)=O)c(CC4CC4)nc32)CC1]

¹H NMR (400 MHz, CDCl₃) δ=8.57 (d, J=2.0 Hz, 1H), 7.84 (s, 1H), 7.71 (dd, J=8.4, 2.4 Hz, 1H), 7.56-7.50 (m, 1H), 7.50-7.43 (m, 2H), 7.36 (d, J=8.4 Hz, 1H), 5.64 (br s, 1H), 5.45 (br s, 1H), 5.35 (s, 2H), 4.41 (d, J=7.2 Hz, 2H), 3.34 (s, 3H), 3.20-3.03 (m, 1H), 2.95 (d, J=6.8 Hz, 2H), 2.10-2.00 (m, 3H), 1.73-1.65 (m, 2H), 1.37-1.28 (m, 1H), 1.22-1.08 (m, 4H), 0.58-0.47 (m, 2H), 0.38-0.32 (m, 2H). ¹⁹F NMR (376 MHz, CDCl₃) δ −76.94. LC-MS: m/z 651.2 (M+H)⁺.

### methyl 3-[(5-carbamoyl-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-inden-1-yl)methyl]-1-pyrrolidinecarboxylate (Compound 325)

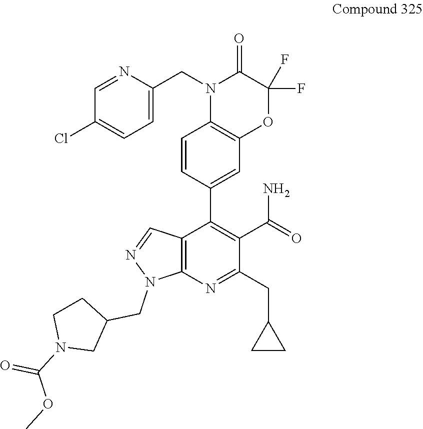 [SMILES: COC(=O)N1CCC(Cn2ncc3c(-c4ccc5c(c4)OC(F)(F)C(=O)N5Cc4ccc(Cl)cn4)c(C(N)=O)c(CC4CC4)nc32)C1]

¹H NMR (400 MHz, CDCl₃) δ 8.56 (d, J=2.4 Hz, 1H), 7.85 (s, 1H), 7.71 (dd, J=8.4, 2.4 Hz, 1H), 7.56-7.50 (m, 1H), 7.48-7.41 (m, 2H), 7.36 (d, J=8.4 Hz, 1H), 5.75-5.62 (m, 1H), 5.59-5.45 (m, 1H), 5.35 (s, 2H), 4.69-4.46 (m, 2H), 3.67 (d, J=4.4 Hz, 3H), 3.61-3.19 (m, 4H), 2.95-2.92 (m, 2H), 2.03-1.94 (m, 1H), 1.34-1.28 (m, 1H), 0.57-0.49 (m, 2H), 0.38-0.28 (m, 2H). ¹⁹F NMR (376 MHz, CDCl₃) δ −76.934. LC-MS: m/z 666.4 (M+H)⁺.

### methyl 3-[(5-carbamoyl-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-inden-1-yl)methyl]-1-azetidinecarboxylate (Compound 326)

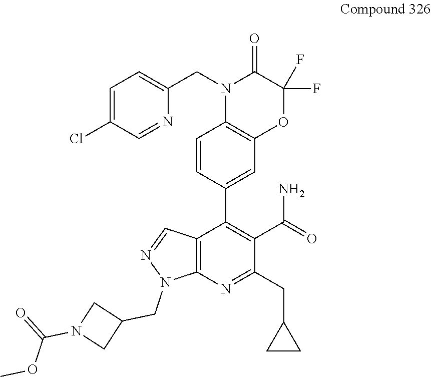 [SMILES: COC(=O)N1CC(Cn2ncc3c(-c4ccc5c(c4)OC(F)(F)C(=O)N5Cc4ccc(Cl)cn4)c(C(N)=O)c(CC4CC4)nc32)C1]

¹H NMR (400 MHz, DMSO-d₆)>8.59 (d, J=2.4 Hz, 1H), 8.01 (dd, J=8.4, 2.4 Hz, 1H), 7.95 (s, 1H), 7.90-7.84 (m, 1H), 7.65-7.56 (m, 3H), 7.53-7.47 (m, 1H), 7.44 (d, J=8.4 Hz, 1H), 5.43 (s, 2H), 4.70 (d, J=6.8 Hz, 2H), 4.07-3.78 (m, 4H), 3.53 (s, 3H), 3.17-3.05 (m, 1H), 2.82 (d, J=6.8 Hz, 2H), 1.30-1.20 (m, 1H), 0.52-0.41 (m, 2H), 0.34-0.26 (m, 2H). ¹⁹F NMR (376 MHz, DMSO-d₆) δ −74.46. LC-MS: m/z 652.2 (M+H)⁺.

### methyl 4-[(5-carbamoyl-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-inden-1-yl)methyl]-1-azepanecarboxylate (Compound 327)

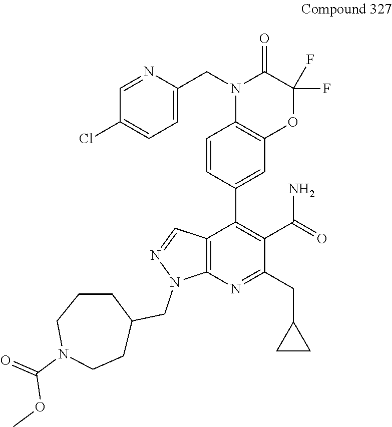 [SMILES: COC(=O)N1CCCC(Cn2ncc3c(-c4ccc5c(c4)OC(F)(F)C(=O)N5Cc4ccc(Cl)cn4)c(C(N)=O)c(CC4CC4)nc32)CC1]

¹H NMR (400 MHz, CD₃OD) δ 8.56-8.46 (m, 1H), 7.91-7.89 (m, 1H), 7.89-7.84 (m, 1H), 7.60-7.55 (m, 1H), 7.50-7.45 (m, 2H), 7.44-7.38 (m, 1H), 5.44 (s, 2H), 4.42 (d, J=7.6 Hz, 2H), 3.69-3.65 (m, 3H), 3.60-3.46 (m, 2H), 3.41-3.34 (m, 1H), 3.29-3.21 (m, 1H), 2.92 (d, J=6.8 Hz, 2H), 2.35-2.18 (m, 1H), 1.91-1.79 (m, 1H), 1.77-1.63 (m, 2H), 1.59-1.40 (m, 2H), 1.38-1.27 (m, 2H), 0.56-0.48 (m, 2H), 0.39-0.30 (m, 2H). LC-MS: m/z 694.3 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(2-morpholinyl)methyl]-1,2,7-triaza-1H-indene-5-carboxamide (Compound 328)

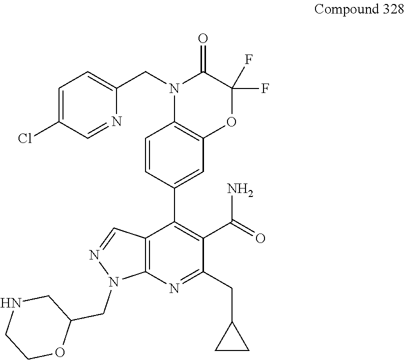 [SMILES: NC(=O)c1c(CC2CC2)nc2c(cnn2CC2CNCCO2)c1-c1ccc2c(c1)OC(F)(F)C(=O)N2Cc1ccc(Cl)cn1]

¹H NMR (400 MHz, CD₃CN) δ 8.52 (d, J=2.4 Hz, 1H), 7.89 (s, 1H), 7.84 (dd, J=8.4, 2.4 Hz, 1H), 7.56 (d, J=1.6 Hz, 1H), 7.51-7.42 (m, 2H), 7.32 (d, J=8.4 Hz, 1H), 6.43 (br.s., 1H), 6.10 (br.s., 1H), 5.36 (s, 2H), 4.71-4.60 (m, 1H), 4.44-4.33 (m, 1H), 4.09-3.93 (m, 1H), 3.91-3.65 (m, 1H), 3.48-3.33 (m, 1H), 3.15-2.42 (m, 6H), 1.36-1.27 (m, 1H), 0.53-0.45 (m, 2H), 0.37-0.29 (m, 2H). ¹⁹F NMR (376 MHz, CD₃CN) δ −76.98. LC-MS: m/z 624.4 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(2-oxa-6-spiro[3.3]heptyl)methyl]-1,2,7-triaza-1H-indene-5-carboxamide (Compound 329)

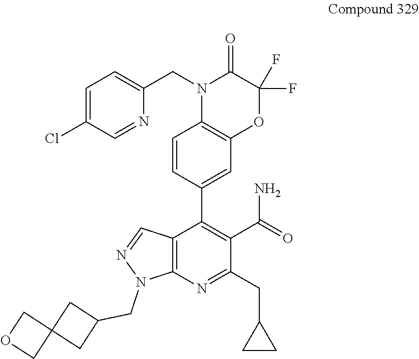 [SMILES: NC(=O)c1c(CC2CC2)nc2c(cnn2CC2CC3(COC3)C2)c1-c1ccc2c(c1)OC(F)(F)C(=O)N2Cc1ccc(Cl)cn1]

¹H NMR (400 MHz, CD₃OD) δ 8.56-8.48 (m, 1H), 7.92-7.82 (m, 2H), 7.60-7.54 (m, 1H), 7.50-7.44 (m, 2H), 7.43-7.37 (m, 1H), 5.43 (s, 2H), 4.66 (s, 2H), 4.60 (s, 2H), 4.50 (d, J=7.2 Hz, 2H), 2.93 (d, J=7.2 Hz, 2H), 2.79-2.68 (m, 1H), 2.37-2.24 (m, 2H), 2.20-2.07 (m, 2H), 1.43-1.30 (m, 1H), 0.58-0.48 (m, 2H), 0.40-0.29 (m, 2H). ¹⁹F NMR (376 MHz, CD₃OD) δ −78.19. LC-MS: m/z 635.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-{[1-(difluoromethyl)-3-pyrazolyl]methyl}-1,2,7-triaza-1H-indene-5-carboxamide (Compound 330)

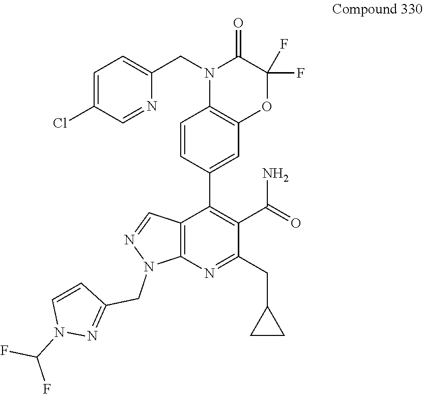 [SMILES: NC(=O)c1c(CC2CC2)nc2c(cnn2Cc2ccn(C(F)F)n2)c1-c1ccc2c(c1)OC(F)(F)C(=O)N2Cc1ccc(Cl)cn1]

¹H NMR (400 MHz, DMSO-d₆) δ 8.59 (d, J=2.0 Hz, 1H), 8.14 (d, J=2.4 Hz, 1H), 8.00 (dd, J=8.4, 2.4 Hz, 1H), 7.96 (s, 1H), 7.88-7.84 (m, 1H), 7.74 (t, J=59.2 Hz, 1H), 7.63-7.57 (m, 3H), 7.52-7.47 (m, 1H), 7.44 (d, J=8.4 Hz, 1H), 6.36 (d, J=2.8 Hz, 1H), 5.70 (s, 2H), 5.43 (s, 2H), 2.82 (d, J=7.2 Hz, 2H), 1.34-1.26 (m, 1H), 0.49-0.42 (m, 2H), 0.33-0.27 (m, 2H). ¹⁹F NMR (376 MHz, DMSO-d₆) δ −74.45, −93.96. LC-MS: m/z 655.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-{1-[1-(difluoromethyl)-3-pyrazolyl]ethyl}-1,2,7-triaza-1H-indene-5-carboxamide (Compound 331)

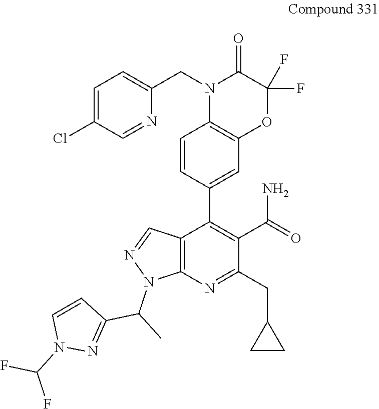 [SMILES: CC(c1ccn(C(F)F)n1)n1ncc2c(-c3ccc4c(c3)OC(F)(F)C(=O)N4Cc3ccc(Cl)cn3)c(C(N)=O)c(CC3CC3)nc21]

¹H NMR (400 MHz, DMSO-d₆)>8.59 (d, J=2.4 Hz, 1H), 8.13 (d, J=2.8 Hz, 1H), 8.00 (dd, J=8.4, 2.4 Hz, 1H), 7.95 (s, 1H), 7.88-7.83 (m, 1H), 7.75 (t, J=59.2 Hz, 1H), 7.64-7.57 (m, 3H), 7.52-7.42 (m, 2H), 6.38 (d, J=2.8 Hz, 1H), 6.36-6.30 (m, 1H), 5.42 (s, 2H), 2.88-2.75 (m, 2H), 1.93 (d, J=7.2 Hz, 3H), 1.34-1.23 (m, 1H), 0.49-0.42 (m, 2H), 0.33-0.26 (m, 2H). ¹⁹F NMR (376 MHz, DMSO-d₆) δ −74.44, −93.83. LC-MS: m/z 669.1 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-{[1-(methoxymethyl)cyclopentyl]methyl}-1,2,7-triaza-1H-indene-5-carboxamide (Compound 332)

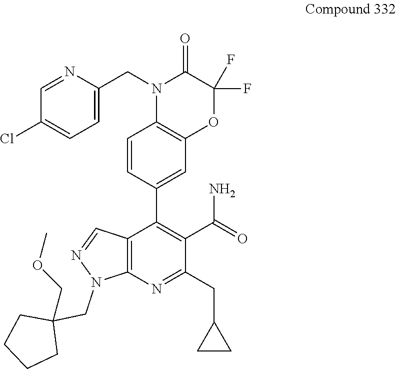 [SMILES: COCC1(Cn2ncc3c(-c4ccc5c(c4)OC(F)(F)C(=O)N5Cc4ccc(Cl)cn4)c(C(N)=O)c(CC4CC4)nc32)CCCC1]

¹H NMR (400 MHz, CD₃OD) δ 8.54-8.50 (m, 1H), 7.91-7.83 (m, 2H), 7.59-7.55 (m, 1H), 7.50-7.48 (m, 1H), 7.48-7.45 (m, 1H), 7.43-7.39 (m, 1H), 5.44 (s, 2H), 4.54 (s, 2H), 3.32 (s, 3H), 3.25 (s, 2H), 2.92 (d, J=7.2 Hz, 2H), 1.77-1.66 (m, 2H), 1.64-1.54 (m, 4H), 1.53-1.42 (m, 2H), 1.41-1.33 (m, 1H), 0.56-0.49 (m, 2H), 0.38-0.31 (m, 2H). ¹⁹F NMR (376 MHz, CD₃OD) δ −78.20. LC-MS: m/z 651.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[1-(tetrahydro-4H-pyran-4-yl)ethyl]-1,2,7-triaza-1H-indene-5-carboxamide (Compound 333)

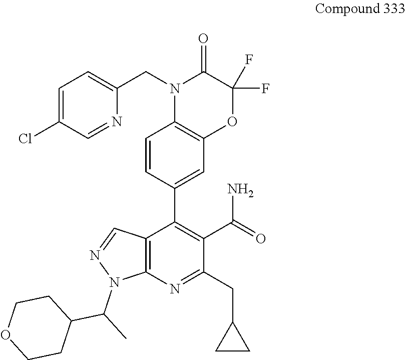 [SMILES: CC(C1CCOCC1)n1ncc2c(-c3ccc4c(c3)OC(F)(F)C(=O)N4Cc3ccc(Cl)cn3)c(C(N)=O)c(CC3CC3)nc21]

¹H NMR (400 MHz, CD₃OD) § 8.52 (d, J=2.4 Hz, 1H), 7.91 (s, 1H), 7.86 (dd, J=8.4, 2.4 Hz, 1H), 7.57 (d, J=1.6 Hz, 1H), 7.50-7.45 (m, 2H), 7.41 (d, J=8.4 Hz, 1H), 5.44 (s, 2H), 4.97-4.90 (m, 1H), 4.04-3.93 (m, 1H), 3.81-3.70 (m, 1H), 3.47-3.37 (m, 1H), 3.28-3.18 (m, 1H), 3.01-2.83 (m, 2H), 2.34-2.19 (m, 1H), 1.93-1.82 (m, 1H), 1.60 (d, J=6.8 Hz, 3H), 1.50-1.32 (m, 2H), 1.31-1.18 (m, 1H), 0.89-0.79 (m, 1H), 0.57-0.48 (m, 2H), 0.40-0.30 (m, 2H). ¹⁹F NMR (376 MHz, CD₃OD) δ −78.20. LC-MS: m/z 637.4 (M+H)⁺.

### methyl 4-[1-(5-carbamoyl-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-inden-1-yl)ethyl]-1-piperidinecarboxylate (Compound 334)

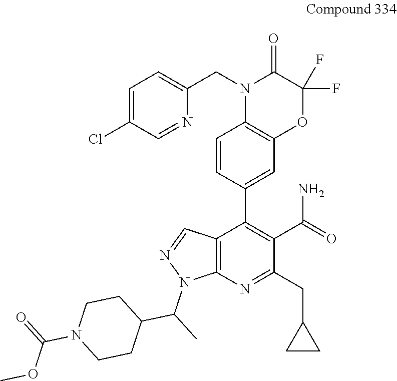 [SMILES: COC(=O)N1CCC(C(C)n2ncc3c(-c4ccc5c(c4)OC(F)(F)C(=O)N5Cc4ccc(Cl)cn4)c(C(N)=O)c(CC4CC4)nc32)CC1]

¹H NMR (400 MHz, DMSO-d₆) δ 8.59 (d, J=2.4 Hz, 1H), 8.00 (dd, J=8.4, 2.4 Hz, 1H), 7.94 (s, 1H), 7.91-7.84 (m, 1H), 7.66-7.55 (m, 3H), 7.49 (dd, J=8.4, 1.6 Hz, 1H), 7.44 (d, J=8.4 Hz, 1H), 5.42 (s, 2H), 4.92-4.79 (m, 1H), 4.06-3.92 (m, 1H), 3.90-3.76 (m, 1H), 3.54 (s, 3H), 2.90-2.71 (m, 3H), 2.69-2.54 (m, 1H), 2.21-2.06 (m, 1H), 1.91-1.80 (m, 1H), 1.52 (d, J=6.8 Hz, 3H), 1.30-1.23 (m, 1H), 1.19-1.09 (m, 1H), 1.06-0.93 (m, 2H), 0.51-0.40 (m, 2H), 0.37-0.24 (m, 2H). ¹⁹F NMR (376 MHz, DMSO-d₆) δ −74.46. LC-MS: m/z 694.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[2-(3,3-difluoro-1-azetidinyl)ethyl]-1,2,7-triaza-1H-indene-5-carboxamide (Compound 335)

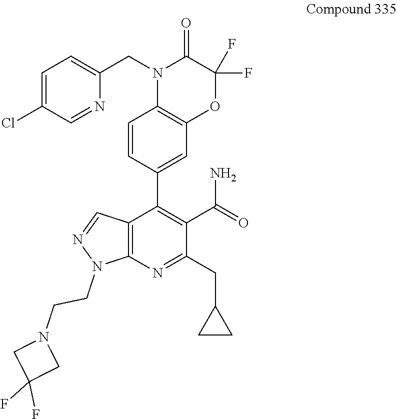 [SMILES: NC(=O)c1c(CC2CC2)nc2c(cnn2CCN2CC(F)(F)C2)c1-c1ccc2c(c1)OC(F)(F)C(=O)N2Cc1ccc(Cl)cn1]

¹H NMR (400 MHz, DMSO-d₆) δ 8.59 (d, J=2.0 Hz, 1H), 8.01 (dd, J=8.4, 2.4 Hz, 1H), 7.93 (s, 1H), 7.90-7.84 (m, 1H), 7.67-7.53 (m, 3H), 7.53-7.47 (m, 1H), 7.44 (d, J=8.4 Hz, 1H), 5.43 (s, 2H), 4.49 (t, J=5.6 Hz, 2H), 3.66-3.51 (m, 4H), 3.11-3.01 (m, 2H), 2.82 (d, J=6.8 Hz, 2H), 1.36-1.25 (m, 1H), 0.52-0.42 (m, 2H), 0.35-0.25 (m, 2H). ¹⁹F NMR (376 MHz, DMSO-d₆) δ −74.45, −97.55. LC-MS: m/z 644.4 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[2-(3,3-difluoro-1-pyrrolidinyl)ethyl]-1,2,7-triaza-1H-indene-5-carboxamide (Compound 336)

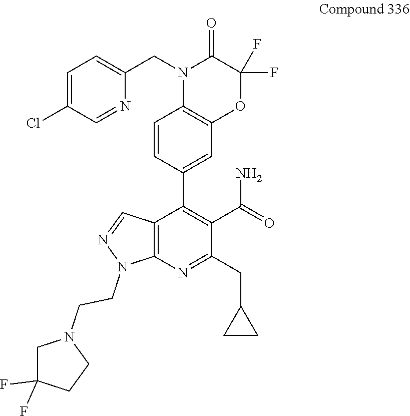 [SMILES: NC(=O)c1c(CC2CC2)nc2c(cnn2CCN2CCC(F)(F)C2)c1-c1ccc2c(c1)OC(F)(F)C(=O)N2Cc1ccc(Cl)cn1]

¹H NMR (400 MHz, CDCl₃) δ 8.57 (d, J=2.4 Hz, 1H), 7.85 (s, 1H), 7.71 (dd, J=8.4, 2.4 Hz, 1H), 7.55-7.51 (m, 1H), 7.50-7.43 (m, 2H), 7.36 (d, J=8.4 Hz, 1H), 5.63 (br s, 1H), 5.45 (br s, 1H), 5.35 (s, 2H), 4.68 (t, J=6.4 Hz, 2H), 3.10 (t, J=6.4 Hz, 2H), 3.02 (t, J=13.6 Hz, 2H), 2.95 (d, J=6.8 Hz, 2H), 2.85 (t, J=6.8 Hz, 2H), 2.27-2.15 (m, 2H), 1.37-1.30 (m, 1H), 0.60-0.49 (m, 2H), 0.38-0.20 (m, 2H). ¹⁹F NMR (377 MHz, CDCl₃) δ −76.95, −92.58. LC-MS: m/z 658.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[2-(3-oxetanyl)ethyl]-1,2,7-triaza-1H-indene-5-carboxamide (Compound 337)

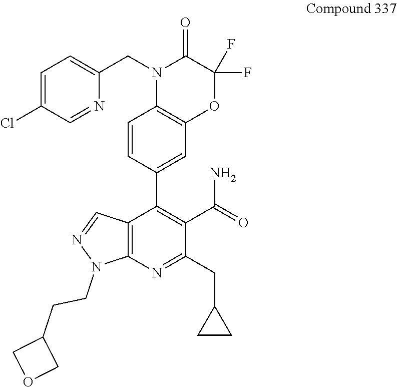 [SMILES: NC(=O)c1c(CC2CC2)nc2c(cnn2CCC2COC2)c1-c1ccc2c(c1)OC(F)(F)C(=O)N2Cc1ccc(Cl)cn1]

¹H NMR (400 MHz, CD₃OD) δ 8.55-8.49 (m, 1H), 7.90 (s, 1H), 7.89-7.84 (m, 1H), 7.59-7.54 (m, 1H), 7.50-7.44 (m, 2H), 7.44-7.39 (m, 1H), 5.44 (s, 2H), 4.63-4.58 (m, 2H), 4.58-4.52 (m, 2H), 4.29-4.16 (m, 2H), 3.00-2.89 (m, 3H), 2.37-2.27 (m, 2H), 1.43-1.31 (m, 1H), 0.58-0.48 (m, 2H), 0.40-0.30 (m, 2H). ¹⁹F NMR (376 MHz, CD₃OD) δ −78.21. LC-MS: m/z 609.2 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-(2-morpholinoethyl)-1,2,7-triaza-1H-indene-5-carboxamide (compound 338)

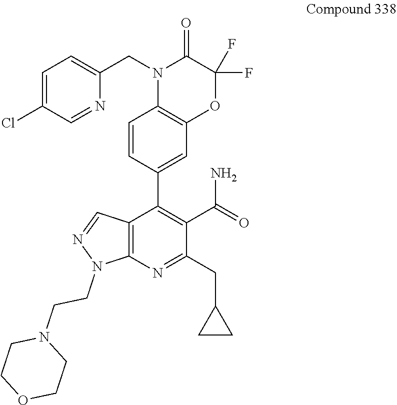 [SMILES: NC(=O)c1c(CC2CC2)nc2c(cnn2CCN2CCOCC2)c1-c1ccc2c(c1)OC(F)(F)C(=O)N2Cc1ccc(Cl)cn1]

¹H NMR (400 MHz, CDCl₃) δ 8.56 (d, J=2.4 Hz, 1H), 7.84 (s, 1H), 7.71 (dd, J=8.4, 2.4 Hz, 1H), 7.56-7.50 (m, 1H), 7.49-7.40 (m, 2H), 7.36 (d, J=8.0 Hz, 1H), 5.77-5.61 (m, 1H), 5.56-5.44 (m, 1H), 5.34 (s, 2H), 4.69 (t, J=1.2 Hz, 2H), 3.68-3.58 (m, 4H), 3.03-2.88 (m, 4H), 2.65-2.48 (m, 4H), 1.39-1.24 (m, 1H), 0.59-0.47 (m, 2H), 0.39-0.27 (m, 2H). ¹⁹F NMR (376 MHz, CDCl₃) δ −76.94. LC-MS: m/z 638.5 (M+H)⁺.

### 1-[2-(2-azetidinyl)ethyl]-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-indene-5-carboxamide (Compound 339)

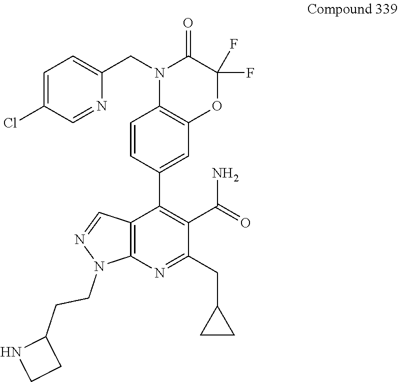 [SMILES: NC(=O)c1c(CC2CC2)nc2c(cnn2CCC2CCN2)c1-c1ccc2c(c1)OC(F)(F)C(=O)N2Cc1ccc(Cl)cn1]

¹H NMR (400 MHz, CDCl₃) δ 8.57 (d, J=2.0 Hz, 1H), 7.84 (s, 1H), 7.71 (dd, J=8.4, 2.4 Hz, 1H), 7.52 (d, J=1.6 Hz, 1H), 7.49-7.42 (m, 2H), 7.36 (d, J=8.4 Hz, 1H), 5.64 (br s, 1H), 5.53 (d, J=1.2 Hz, 1H), 5.34 (s, 2H), 4.78-4.59 (m, 1H), 4.57-4.40 (m, 1H), 3.76-3.63 (m, 1H), 3.62-3.48 (m, 1H), 3.36-3.25 (m, 1H), 3.04-2.85 (m, 2H), 2.29-1.98 (m, 5H), 1.39-1.30 (m, 1H), 0.60-0.49 (m, 2H), 0.38-0.28 (m, 2H). ¹⁹F NMR (376 MHz, CDCl₃) δ −76.94. LC-MS: m/z 608.3 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[2-(1-piperazinyl)ethyl]-1,2,7-triaza-1H-indene-5-carboxamide (Compound 340)

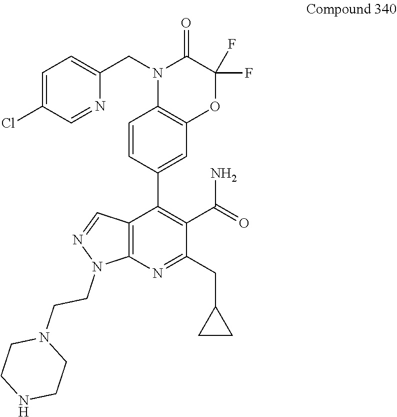 [SMILES: NC(=O)c1c(CC2CC2)nc2c(cnn2CCN2CCNCC2)c1-c1ccc2c(c1)OC(F)(F)C(=O)N2Cc1ccc(Cl)cn1]

¹H NMR (400 MHz, CDCl₃) δ 8.55 (d, J=2.0 Hz, 1H), 8.19-8.04 (m, 1H), 7.83 (s, 1H), 7.71 (dd, J=8.4, 2.4 Hz, 1H), 7.54-7.49 (m, 1H), 7.49-7.40 (m, 2H), 7.35 (d, J=8.4 Hz, 1H), 6.72 (br.s., 1H), 5.88 (br.s., 1H), 5.34 (s, 2H), 4.67 (t, J=6.0 Hz, 2H), 3.02 (t, J=6.0 Hz, 2H), 2.92 (d, J=6.8 Hz, 2H), 2.82-2.76 (m, 4H), 2.65-2.56 (m, 4H), 1.36-1.23 (m, 1H), 0.56-0.45 (m, 2H), 0.36-0.26 (m, 2H). ¹⁹F NMR (376 MHz, CDCl₃) δ −76.83. LC-MS: m/z 637.4 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[2-(3-morpholinyl)ethyl]-1,2,7-triaza-1H-indene-5-carboxamide (Compound 341)

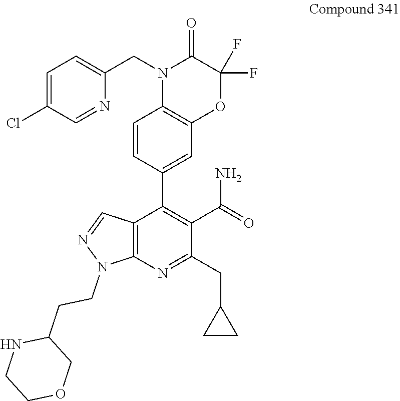 [SMILES: NC(=O)c1c(CC2CC2)nc2c(cnn2CCC2COCCN2)c1-c1ccc2c(c1)OC(F)(F)C(=O)N2Cc1ccc(Cl)cn1]

¹H NMR (400 MHz, CDCl₃) δ 8.57 (d, J=2.0 Hz, 1H), 7.90 (s, 1H), 7.72 (dd, J=2.4, 8.4 Hz, 1H), 7.51 (s, 1H), 7.46 (s, 2H), 7.36 (d, J=8.0 Hz, 1H), 5.90 (br s, 1H), 5.78 (br s, 1H), 5.35 (s, 2H), 4.85-4.79 (m, 1H), 4.74-4.67 (m, 1H), 4.03-3.91 (m, 3H), 3.83-3.73 (m, 1H), 3.46 (br dd, J=1.6, 12.4 Hz, 1H), 3.20-3.03 (m, 2H), 2.99-2.86 (m, 2H), 2.53-2.41 (m, 1H), 2.32-2.20 (m, 1H), 1.30-1.23 (m, 1H), 0.55 (br d, J=7.6 Hz, 2H), 0.33 (br d, J=4.0 Hz, 2H). ¹⁹F NMR (377 MHz, CDCl₃) δ −75.79. LC-MS: m/z 638.3 (M+H)⁺.

### 2,2-difluoroethyl(5-carbamoyl-4-{4-[(5-chloro-2-pyridyl)methyl]-2,2-difluoro-3-oxo-3,4-dihydro-2H-1,4-benzoxazin-7-yl}-6-(cyclopropylmethyl)-1,2,7-triaza-1H-inden-1-yl)acetate (Compound 342)

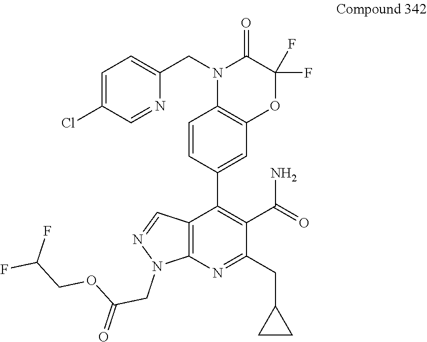 [SMILES: NC(=O)c1c(CC2CC2)nc2c(cnn2CC(=O)OCC(F)F)c1-c1ccc2c(c1)OC(F)(F)C(=O)N2Cc1ccc(Cl)cn1]

¹H NMR (400 MHz, DMSO-d₆) δ 8.59 (d, J=2.4 Hz, 1H), 8.03 (s, 1H), 8.00 (dd, J=8.4, 2.4 Hz, 1H), 7.95-7.89 (m, 1H), 7.67-7.57 (m, 3H), 7.53-7.42 (m, 2H), 6.46-6.14 (m, 1H), 5.49 (s, 2H), 5.44 (s, 2H), 4.51-4.40 (m, 2H), 2.80 (d, J=6.8 Hz, 2H), 1.31-1.24 (m, 1H), 0.47-0.39 (m, 2H), 0.34-0.21 (m, 2H). ¹⁹F NMR (376 MHz, DMSO-d₆) δ −74.44, −126.19. LC-MS: m/z 647.1 (M+H)⁺.

### methyl 3-[(6-carbamoyl-7-{4-[(5-chloro-2-pyrimidinyl)methyl]-2,2-difluoro-3-oxo-3,4-dihydro-2H-1,4-benzoxazin-7-yl}-5-(cyclopropylmethyl)-3-methyl-1-oxo-2-oxa-4-aza-3-indanyl)methyl]-1-azetidinecarboxylate (Compound 348)

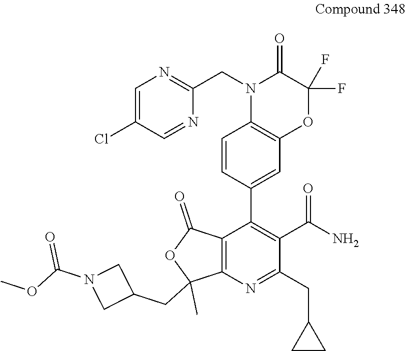 [SMILES: COC(=O)N1CC(CC2(C)OC(=O)c3c2nc(CC2CC2)c(C(N)=O)c3-c2ccc3c(c2)OC(F)(F)C(=O)N3Cc2ncc(Cl)cn2)C1]

¹H NMR (400 MHz, CDCl₃) δ 8.61 (s, 2H), 7.34-7.29 (m, 1H), 7.19-7.14 (m, 1H), 6.95-6.87 (m, 1H), 5.60 (s, 2H), 5.36 (s, 2H), 4.00-3.91 (m, 1H), 3.68-3.56 (m, 2H), 3.53 (s, 3H), 3.43-3.24 (m, 1H), 2.89-2.77 (m, 2H), 2.58-2.45 (m, 1H), 2.37-2.28 (m, 2H), 1.61 (s, 3H), 1.25-1.20 (m, 1H), 0.51-0.42 (m, 2H), 0.31-0.22 (m, 2H). ¹⁹F NMR (376 MHz, CDCl₃) δ −76.22. LC-MS: m/z 683.2 (M+H)⁺.

### methyl 4-[(6-carbamoyl-7-{4-[(5-chloro-2-pyrimidinyl)methyl]-2,2-difluoro-3-oxo-3,4-dihydro-2H-1,4-benzoxazin-7-yl}-5-(cyclopropylmethyl)-3-methyl-1-oxo-2-oxa-4-aza-3-indanyl)methyl]-1-piperidinecarboxylate (Compound 349)

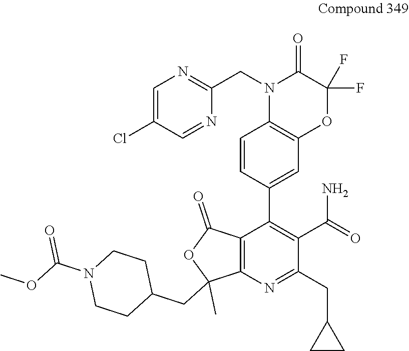 [SMILES: COC(=O)N1CCC(CC2(C)OC(=O)c3c2nc(CC2CC2)c(C(N)=O)c3-c2ccc3c(c2)OC(F)(F)C(=O)N3Cc2ncc(Cl)cn2)CC1]

¹H NMR (400 MHz, CD₃OD) § 8.79 (s, 2H), 7.44-7.40 (m, 1H), 7.30-7.24 (m, 1H), 7.22-7.17 (m, 1H), 5.51 (s, 2H), 4.07-3.86 (m, 2H), 3.64 (s, 3H), 2.96-2.83 (m, 2H), 2.82-2.59 (m, 2H), 2.18-2.08 (m, 1H), 2.05-1.95 (m, 1H), 1.70-1.68 (m, 4H), 1.56-1.46 (m, 1H), 1.45-1.37 (m, 1H), 1.35-1.25 (m, 1H), 1.25-1.12 (m, 1H), 1.12-0.98 (m, 1H), 0.56-0.48 (m, 2H), 0.39-0.30 (m, 2H). ¹⁹F NMR (376 MHz, CD₃OD) δ −78.32. LC-MS: m/z 711.3 (M+H)⁺.

### 7-{4-[(5-chloro-2-pyrimidinyl)methyl]-2,2-difluoro-3-oxo-3,4-dihydro-2H-1,4-benzoxazin-7-yl}-5-(cyclopropylmethyl)-3-[(5-fluoro-2-pyridyl)methyl]-3-methyl-1-oxo-2-oxa-4-aza-6-indancarboxamide (Compound 350)

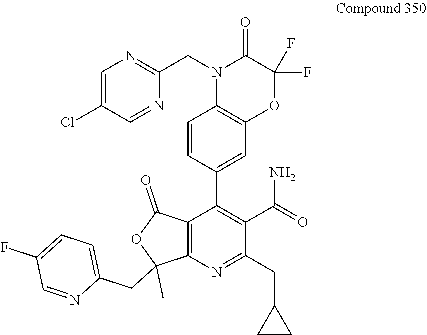 [SMILES: CC1(Cc2ccc(F)cn2)OC(=O)c2c1nc(CC1CC1)c(C(N)=O)c2-c1ccc2c(c1)OC(F)(F)C(=O)N2Cc1ncc(Cl)cn1]

¹H NMR (400 MHz, CDCl₃) δ 8.68 (s, 2H), 8.29 (d, J=2.8 Hz, 1H), 7.37-7.32 (m, 1H), 7.31 (d, J=1.6 Hz, 1H), 7.24 (m, 1H), 7.16 (dd, J=1.6, 8.4 Hz, 1H), 6.96 (d, J=8.4 Hz, 1H), 5.59 (br s, 1H), 5.47 (br s, 1H), 5.42 (s, 2H), 3.63-3.56 (m, 1H), 3.51-3.46 (m, 1H), 2.93 (dd, J=2.0, 6.8 Hz, 2H), 1.76 (s, 3H), 1.28 (m, 1H), 0.56-0.52 (m, 2H), 0.38-0.34 (m, 2H). ¹⁹F NMR (376 MHz, CDCl₃) δ −77.23. LC-MS: m/z 665.2 (M−H)⁺.

### 4-{4-[(5-chloro-2-pyridyl)methyl]-2,2-dimethyl-3-oxo-3,4-dihydro-2H-1,4-benzoxazin-7-yl}-6-isobutyl-1-[(S)-2,2,2-trifluoro-1-methylethyl]-1,2,7-triaza-1H-indene-5-carboxamide and 4-{4-[(5-chloro-2-pyridyl)methyl]-2,2-dimethyl-3-oxo-3,4-dihydro-2H-1,4-benzoxazin-7-yl}-6-isobutyl-1-[(R)-2,2,2-trifluoro-1-methylethyl]-1,2,7-triaza-1H-indene-5-carboxamide (Compound 351 and Compound 352)

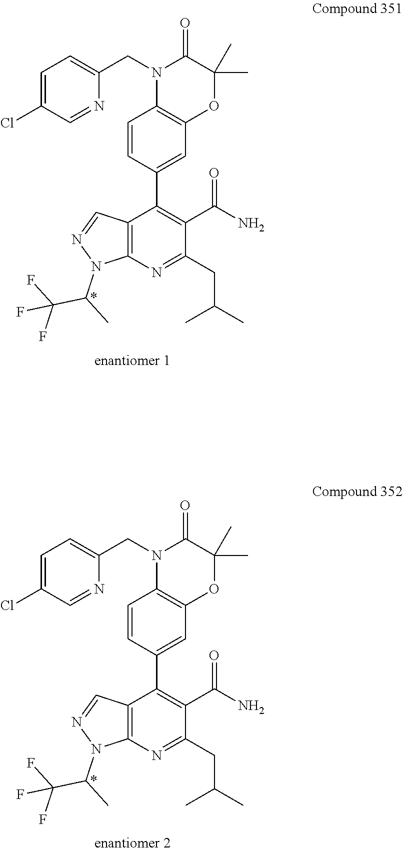 [SMILES: CC(C)Cc1nc2c(cnn2C(C)C(F)(F)F)c(-c2ccc3c(c2)OC(C)(C)C(=O)N3Cc2ccc(Cl)cn2)c1C(N)=O ; CC(C)Cc1nc2c(cnn2C(C)C(F)(F)F)c(-c2ccc3c(c2)OC(C)(C)C(=O)N3Cc2ccc(Cl)cn2)c1C(N)=O]

Compound 189 (160 mg) was purified by SFC (Column name: DAICEL CHIRALPAK® ID; Column size: 250*25 mm 10 μm; Mobile Phase A: n-Hexane; Mobile Phase B: EtOH (+0.1% 7.0 mol/l Ammonia in MEOH); Gradient: A/B=90/10; Flow rate: 50 mL/min, Column temp: RT) to afford 4-{4-[(5-chloro-2-pyridyl)methyl]-2,2-dimethyl-3-oxo-3,4-dihydro-2H-1,4-benzoxazin-7-yl}-6-isobutyl-1-[(S)-2,2,2-trifluoro-1-methylethyl]-1,2,7-triaza-1H-indene-5-carboxamide and 4-{4-[(5-chloro-2-pyridyl)methyl]-2,2-dimethyl-3-oxo-3,4-dihydro-2H-1,4-benzoxazin-7-yl}-6-isobutyl-1-[(R)-2,2,2-trifluoro-1-methylethyl]-1,2,7-triaza-1H-indene-5-carboxamide.

Compound 351, enantiomer 1 (16.51 mg, 8.46%): Rt=11.21 min. ¹H NMR (400 MHz, DMSO-d₆) δ 8.59 (d, J=2.4 Hz, 1H), 8.06 (s, 1H), 7.95 (dd, J₁=2.4 Hz, J₂=8.4 Hz, 1H), 7.76 (m, 1H), 7.55 (m, 1H), 7.40 (d, J=8.8 Hz, 1H), 7.30 (d, J=2 Hz, 1H), 7.26 (dd, J₁=2 Hz, J₂=8.4 Hz, 1H), 7.14 (d, J=8.4 Hz, 1H), 5.78-5.86 (m, 1H), 5.25 (s, 2H), 2.74-2.87 (m, 2H), 2.32-2.39 (m, 1H), 1.81 (d, J=6.8 Hz, 3H), 1.51 (s, 6H), 0.94 (dd, 1=2.8 Hz, J₂=6.4 Hz, 6H). ¹⁹F NMR (377 MHz, DMSO-d₆) δ −74.08. LC-MS: m/z 615.5 (M+H)⁺.

Compound 352, enantiomer 2 (16.89 mg, 8.65%): Rt=13.57 min. ¹H NMR (400 MHz, DMSO-d₆) δ 8.59 (d, J=2.4 Hz, 1H), 8.06 (s, 1H), 7.95 (dd, J₁=2.4 Hz, J₂=8.4 Hz, 1H), 7.76 (m, 1H), 7.55 (m, 1H), 7.40 (d, J=8.4 Hz, 1H), 7.30 (d, J=2 Hz, 1H), 7.26 (dd, J₁=2 Hz, J₂=8.4 Hz, 1H), 7.14 (d, J=8.4 Hz, 1H), 5.78-5.86 (m, 1H), 5.25 (s, 2H), 2.74-2.87 (m, 2H), 2.32-2.39 (m, 1H), 1.81 (d, J=7.2 Hz, 3H), 1.51 (s, 6H), 0.94 (dd, J₁=2.8 Hz, J₂=6.4 Hz, 6H). ¹⁹F NMR (377 MHz, DMSO-d₆) δ −74.08. LC-MS: m/z 615.5 (M+H)⁺.

### 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(R)-1-(tetrahydro-4H-pyran-4-yl)ethyl]-1,2,7-triaza-1H-indene-5-carboxamide and 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-(tetrahydro-4H-pyran-4-yl)ethyl]-1,2,7-triaza-1H-indene-5-carboxamide (Compound 353 and Compound 354)

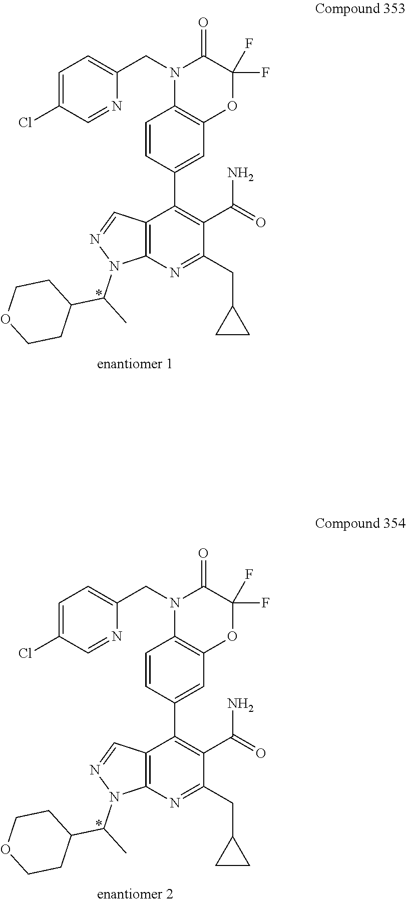 [SMILES: CC(C1CCOCC1)n1ncc2c(-c3ccc4c(c3)OC(F)(F)C(=O)N4Cc3ccc(Cl)cn3)c(C(N)=O)c(CC3CC3)nc21 ; CC(C1CCOCC1)n1ncc2c(-c3ccc4c(c3)OC(F)(F)C(=O)N4Cc3ccc(Cl)cn3)c(C(N)=O)c(CC3CC3)nc21]

Compound 333 (90 mg) was purified by SFC (system: Waters SFC 27; Column name: DAICEL CHIRALPAK AS; Column size: (250 mm*30 mm, 10 μm); mobile phase A: Supercritical CO₂; mobile phase B: MeOH (+0.1% NH₃·H₂O)]; Gradient: A/B=45/55; Flow rate: 130 mL/min; Column Temp: RT) to afford 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(R)-1-(tetrahydro-4H-pyran-4-yl)ethyl]-1,2,7-triaza-1H-indene-5-carboxamide and 4-{4-[(5-chloro-2-pyridyl)methyl]-2,2-difluoro-3-oxo-3,4-dihydro-2H-1,4-benzoxazin-7-yl}-6-(cyclopropylmethyl)-1-[(S)-1-(tetrahydro-4H-pyran-4-yl)ethyl]-1,2,7-triaza-1H-indene-5-carboxamide.

Compound 353, enantiomer 1 (39.48 mg, 43.87%): Rt=1.21 min. ¹H NMR (400 MHz, CD₃OD) δ 8.52 (d, J=2.4 Hz, 1H), 7.91 (s, 1H), 7.85 (dd, J=8.4, 2.4 Hz, 1H), 7.57 (d, J=1.6 Hz, 1H), 7.51-7.44 (m, 2H), 7.40 (d, J=8.4 Hz, 1H), 5.44 (s, 2H), 4.97-4.90 (m, 1H), 4.07-3.91 (m, 1H), 3.83-3.70 (m, 1H), 3.47-3.36 (m, 1H), 3.27-3.17 (m, 1H), 2.99-2.82 (m, 2H), 2.34-2.19 (m, 1H), 1.94-1.81 (m, 1H), 1.61 (d, J=6.8 Hz, 3H), 1.46-1.31 (m, 2H), 1.30-1.21 (m, 1H), 0.91-0.78 (m, 1H), 0.61-0.45 (m, 2H), 0.41-0.26 (m, 2H). ¹⁹F NMR (376 MHz, CD₃OD) δ −78.20. LC-MS: m/z 637.4 (M+H)⁺.

Compound 354, enantiomer 2 (35.93 mg, 39.92%): Rt=2.11 min. ¹H NMR (400 MHz, CD₃OD) δ=8.52 (d, J=2.4 Hz, 1H), 7.91 (s, 1H), 7.87 (dd, J=8.4, 2.4 Hz, 1H), 7.57 (d, J=1.6 Hz, 1H), 7.52-7.44 (m, 2H), 7.41 (d, J=8.4 Hz, 1H), 5.44 (s, 2H), 4.97-4.91 (m, 1H), 4.05-3.92 (m, 1H), 3.83-3.69 (m, 1H), 3.46-3.36 (m, 1H), 3.27-3.16 (m, 1H), 3.03-2.80 (m, 2H), 2.32-2.20 (m, 1H), 1.94-1.82 (m, 1H), 1.60 (d, J=7.2 Hz, 3H), 1.44-1.32 (m, 2H), 1.26-1.16 (m, 1H), 0.90-0.78 (m, 1H), 0.57-0.47 (m, 2H), 0.40-0.30 (m, 2H). ¹⁹F NMR (376 MHz, CD₃OD) δ −78.20. LC-MS: m/z 637.4 (M+H)⁺.

### 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(S)-1-(5-fluoro-2-pyrimidinyl)ethyl]-1,2,7-triaza-1H-indene-5-carboxamide and 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(R)-1-(5-fluoro-2-pyrimidinyl)ethyl]-1,2,7-triaza-1H-indene-5-carboxamide (Compound 355 and Compound 356)

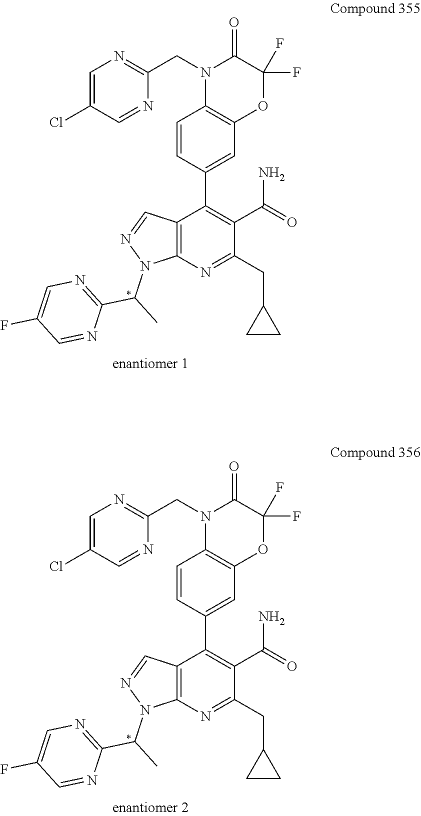 [SMILES: CC(c1ncc(F)cn1)n1ncc2c(-c3ccc4c(c3)OC(F)(F)C(=O)N4Cc3ncc(Cl)cn3)c(C(N)=O)c(CC3CC3)nc21 ; CC(c1ncc(F)cn1)n1ncc2c(-c3ccc4c(c3)OC(F)(F)C(=O)N4Cc3ncc(Cl)cn3)c(C(N)=O)c(CC3CC3)nc21]

4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(1-(5-fluoro-2-pyrimidinyl)ethyl]-1,2,7-triaza-1H-indene-5-carboxamide which was synthesized following the similar route of Example 56 (176 mg) was purified by SFC (system: Waters SFC 150; Column name: DAICEL CHIRALPAK® IB; Column size: 250*25 mm 10 μm; Mobile Phase A: Supercritical CO₂; Mobile Phase B: MEOH (+0.1% 7.0 mol/l Ammonia in MEOH); Gradient: A/B=75/25; Flow rate: 120 mL/min; Column Temp: RT) to afford 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(S)-1-(5-fluoro-2-pyrimidinyl)ethyl]-1,2,7-triaza-1H-indene-5-carboxamide and 4-{4-[(5-chloro-2-pyrimidinyl)methyl]-2,2-difluoro-3-oxo-3,4-dihydro-2H-1,4-benzoxazin-7-yl}-6-(cyclopropylmethyl)-1-[(R)-1-(5-fluoro-2-pyrimidinyl)ethyl]-1,2,7-triaza-1H-indene-5-carboxamide.

Compound 355, enantiomer 1 (29.91 mg, 17.04%): Rt=2.66 min. ¹H NMR (400 MHz, DMSO-d₆) δ 8.98 (s, 2H), 8.84 (s, 2H), 7.94 (s, 1H), 7.86 (s, 1H), 7.63 (d, J=1.6 Hz, 1H), 7.60 (s, 1H), 7.49 (dd, J₁=1.6 Hz, J₂=8.4 Hz, 1H), 7.44 (d, J=8.0 Hz, 1H), 6.36-6.41 (m, 1H), 5.51 (s, 2H), 2.72-2.75 (m, 2H), 2.01 (d, J=6.8 Hz, 3H), 1.15-1.18 (m, 1H), 0.35-0.37 (m, 2H), 0.17-0.21 (m, 2H). ¹⁹F NMR (376 MHz, DMSO-d₆) δ −74.84, −140.00. LC-MS: m/z 650.2 (M+H)⁺.

Compound 356, enantiomer 2 (19.18 mg, 10.93%): Rt=3.33 min. ¹H NMR (400 MHz, DMSO-d₆) δ 8.98 (s, 2H), 8.84 (s, 2H), 7.94 (s, 1H), 7.86 (s, 1H), 7.63 (d, J=1.6 Hz, 1H), 7.60 (s, 1H), 7.49 (dd, J₁=1.6 Hz, J₂=8.4 Hz, 1H), 7.44 (d, J=8.4 Hz, 1H), 6.36-6.42 (m, 1H), 5.51 (s, 2H), 2.72-2.75 (m, 2H), 2.01 (d, J=7.2 Hz, 3H), 1.12-1.16 (m, 1H), 0.34-0.37 (m, 2H), 0.16-0.21 (m, 2H). ¹⁹F NMR (376 MHz, DMSO-d₆) δ −74.85, −140.00. LC-MS: m/z 650.2 (M+H)⁺.

### 4-[(S)-4-[(5-chloro-2-pyrimidinyl)methyl]-2-fluoro-3-oxo-3,4-dihydro-2H-1,4-benzoxazin-7-yl]-6-(cyclopropylmethyl)-1-[1-(5-fluoro-2-pyridyl)cyclopropyl]-1,2,7-triaza-1H-indene-5-carboxamide and 4-[(R)-4-[(5-chloro-2-pyrimidinyl)methyl]-2-fluoro-3-oxo-3,4-dihydro-2H-1,4-benzoxazin-7-yl]-6-(cyclopropylmethyl)-1-[1-(5-fluoro-2-pyridyl)cyclopropyl]-1,2,7-triaza-1H-indene-5-carboxamide (Compound 357 and Compound 358)

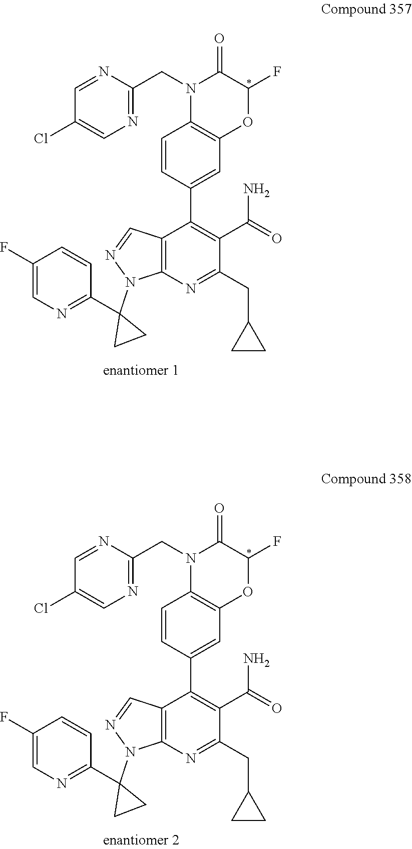 [SMILES: NC(=O)c1c(CC2CC2)nc2c(cnn2C2(c3ccc(F)cn3)CC2)c1-c1ccc2c(c1)OC(F)C(=O)N2Cc1ncc(Cl)cn1 ; NC(=O)c1c(CC2CC2)nc2c(cnn2C2(c3ccc(F)cn3)CC2)c1-c1ccc2c(c1)OC(F)C(=O)N2Cc1ncc(Cl)cn1]

Compound 303 (38.27 mg) was purified by SFC (system: CASSH-SFC-21; Column name: REGIS (R,R) WHELK-01; Column size: 250 mm*25 mm, 10 μm; Mobile Phase A: Supercritical CO₂; Mobile Phase B: IPA (+0.1% NH₃H₂O); Gradient: A/B=50/50; Flow rate: 80 mL/min; Column Temp: RT) to afford 4-[(S)-4-[(5-chloro-2-pyrimidinyl)methyl]-2-fluoro-3-oxo-3,4-dihydro-2H-1,4-benzoxazin-7-yl]-6-(cyclopropylmethyl)-1-[1-(5-fluoro-2-pyridyl)cyclopropyl]-1,2,7-triaza-1H-indene-5-carboxamide and 4-[(R)-4-[(5-chloro-2-pyrimidinyl)methyl]-2-fluoro-3-oxo-3,4-dihydro-2H-1,4-benzoxazin-7-yl]-6-(cyclopropylmethyl)-1-[1-(5-fluoro-2-pyridyl)cyclopropyl]-1,2,7-triaza-1H-indene-5-carboxamide.

Compound 357, enantiomer 1 (8.35 mg, 21.96%): Rt=2.78 min. ¹H NMR (400 MHz, CD₃OD) δ 8.80 (s, 2H), 8.37 (d, J=2.8 Hz, 1H), 8.03 (s, 1H), 7.57 (d, J=2.0 Hz, 1H), 7.43 (dd, J=8.4, 2.0 Hz, 1H), 7.25-7.33 (m, 2H), 6.25-6.39 (m, 2H), 5.62 (d, J=17.6 Hz, 1H), 5.42 (d. J=17.6 Hz, 1H), 2.86 (d, J=7.2 Hz, 2H), 2.02-2.05 (m, 2H), 1.86-1.90 (m, 2H), 0.86-0.92 (m, 1H), 0.40-0.44 (m, 2H), 0.24-0.28 (m, 2H). ¹⁹F NMR (376 MHz, CD₃OD) δ −130.02, −133.58. LC-MS: m/z 643.2 (M+H)⁺.

Compound 358, enantiomer 2 (7.26 mg, 18.97%): Rt-3.26 min. ¹H NMR (400 MHz, CD₃OD) δ 8.79 (s, 2H), 8.36 (d, J=2.8 Hz, 1H), 8.02 (s, 1H), 7.56 (d, J=1.6 Hz, 1H), 7.42 (dd, J=8.4, 2.0 Hz, 1H), 7.24-7.32 (m, 2H), 6.25-6.38 (m, 2H), 5.61 (d, J=17.6 Hz, 1H), 5.41 (d. J=17.6 Hz, 1H), 2.86 (d, J=7.2 Hz, 2H), 2.00-2.04 (m, 2H), 1.86-1.90 (m, 2H), 0.79-0.97 (m, 1H), 0.39-0.44 (m, 2H), 0.23-0.27 (m, 2H). ¹⁹F NMR (376 MHz, CD₃OD) δ −130.01, −133.57. LC-MS: m/z 643.3 (M+H)⁺.

### 6-(cyclopropylmethyl)-4-[(S)-2-fluoro-4-[(5-fluoro-2-pyrimidinyl)methyl]-3-oxo-3,4-dihydro-2H-1,4-benzoxazin-7-yl]-1-[1-(5-fluoro-2-pyridyl)cyclopropyl]-1,2,7-triaza-1H-indene-5-carboxamide and 6-(cyclopropylmethyl)-4-[(R)-2-fluoro-4-[(5-fluoro-2-pyrimidinyl)methyl]-3-oxo-3,4-dihydro-2H-1,4-benzoxazin-7-yl]-1-[1-(5-fluoro-2-pyridyl)cyclopropyl]-1,2,7-triaza-1H-indene-5-carboxamide (Compound 359 and Compound 360)

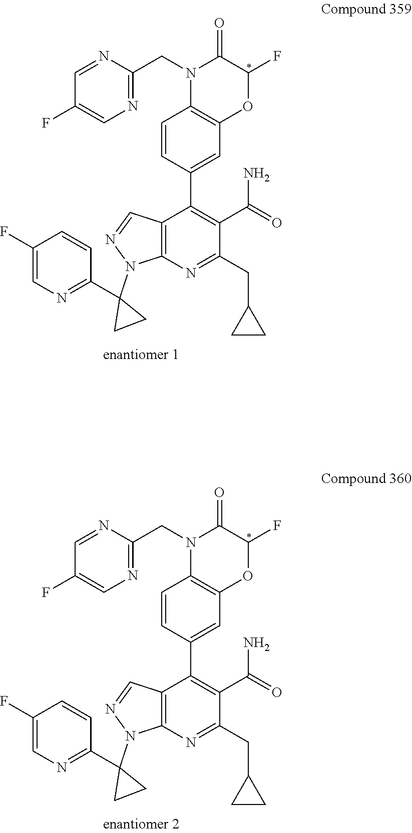 [SMILES: NC(=O)c1c(CC2CC2)nc2c(cnn2C2(c3ccc(F)cn3)CC2)c1-c1ccc2c(c1)OC(F)C(=O)N2Cc1ncc(F)cn1 ; NC(=O)c1c(CC2CC2)nc2c(cnn2C2(c3ccc(F)cn3)CC2)c1-c1ccc2c(c1)OC(F)C(=O)N2Cc1ncc(F)cn1]

6-(cyclopropylmethyl)-4-[(2-fluoro-4-[(5-fluoro-2-pyrimidinyl)methyl]-3-oxo-3,4-dihydro-2H-1,4-benzoxazin-7-yl]-1-[1-(5-fluoro-2-pyridyl)cyclopropyl]-1,2,7-triaza-1H-indene-5-carboxamide which was synthesized following the similar route of Example 56 (140.0 mg) was separated by prep-SFC (system: CASSH-SFC-21; Column name: REGIS(S, S) WHELK-01; Column size: (250 mm*25 mm, 10 um); mobile phase A: Supercritical CO₂; mobile phase B: MeOH (+0.1% NH₃H₂O)]; Gradient: A/B=60/40; Flow rate: 80 mL/min; Column Temp: 40° C.) to afford to afford 6-(cyclopropylmethyl)-4-[(S)-2-fluoro-4-[(5-fluoro-2-pyrimidinyl)methyl]-3-oxo-3,4-dihydro-2H-1,4-benzoxazin-7-yl]-1-[1-(5-fluoro-2-pyridyl)cyclopropyl]-1,2,7-triaza-1H-indene-5-carboxamide and 6-(cyclopropylmethyl)-4-[(R)-2-fluoro-4-[(5-fluoro-2-pyrimidinyl)methyl]-3-oxo-3,4-dihydro-2H-1,4-benzoxazin-7-yl]-1-[1-(5-fluoro-2-pyridyl)cyclopropyl]-1,2,7-triaza-1H-indene-5-carboxamide.

Compound 359, enantiomer 1 (30.15 mg, 21.54%): Rt=1.76 min. ¹H NMR (400 MHz, CDCl₃) δ 8.62 (s, 2H), 8.37 (d, J=2.8 Hz, 1H), 7.98 (s, 1H), 7.54 (d, J=2.0 Hz, 1H), 7.40 (dd, J=8.4, 1.6 Hz, 1H), 7.18-7.03 (m, 2H), 6.38-6.16 (m, 2H), 5.68-5.59 (m, 2H), 5.47-5.39 (m, 1H), 5.37-5.28 (m, 1H), 2.90 (d, J=7.2 Hz, 2H), 2.13-2.02 (m, 2H), 1.98-1.83 (m, 2H), 1.33-1.18 (m, 1H), 0.52-0.38 (m, 2H), 0.33-0.22 (m, 2H). ¹⁹F NMR (376 MHz, CDCl₃) δ −127.99, −131.54, −138.16. LC-MS: m/z 627.1 (M+H)⁺.

Compound 360, enantiomer 2 (33.95 mg, 24.25%): Rt=1.98 min. ¹H NMR (400 MHz, CDCl₃) δ 8.61 (s, 2H), 8.37 (d, J=2.8 Hz, 1H), 7.98 (s, 1H), 7.54 (d, J=1.6 Hz, 1H), 7.40 (dd, J=8.4, 2.0 Hz, 1H), 7.18-7.05 (m, 2H), 6.36-6.14 (m, 2H), 5.71-5.56 (m, 2H), 5.50-5.40 (m, 1H), 5.37-5.25 (m, 1H), 2.96 (d, J=6.8 Hz, 2H), 2.12-2.04 (m, 2H), 1.95-1.86 (m, 2H), 1.30-1.23 (m, 1H), 0.49-0.40 (m, 2H), 0.31-0.24 (m, 2H). ¹⁹F NMR (376 MHz, CDCl₃) δ −127.98, −131.51, −138.16. LC-MS: m/z 627.1 (M+H)⁺.

### 4-[(S)-4-[(5-chloro-2-pyrimidinyl)methyl]-2-fluoro-3-oxo-3,4-dihydro-2H-1,4-benzoxazin-7-yl]-6-(cyclopropylmethyl)-1-[1-(trifluoromethyl)cyclopropyl]-1,2,7-triaza-1H-indene-5-carboxamide and 4-[(R)-4-[(5-chloro-2-pyrimidinyl)methyl]-2-fluoro-3-oxo-3,4-dihydro-2H-1,4-benzoxazin-7-yl]-6-(cyclopropylmethyl)-1-[1-(trifluoromethyl)cyclopropyl]-1,2,7-triaza-1H-indene-5-carboxamide (Compound 361 and Compound 362)

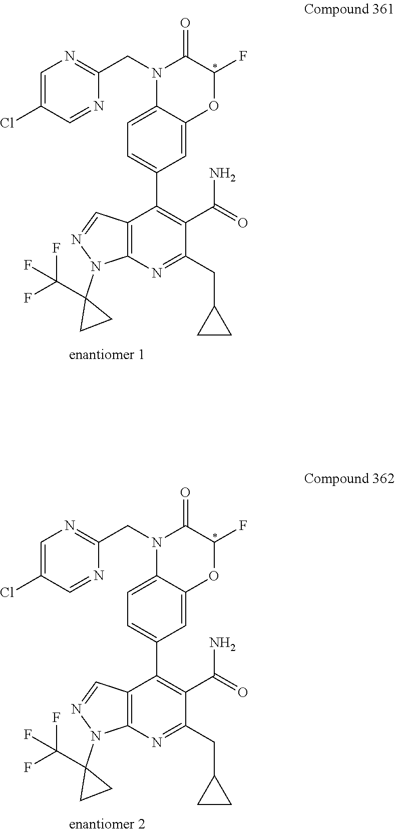 [SMILES: NC(=O)c1c(CC2CC2)nc2c(cnn2C2(C(F)(F)F)CC2)c1-c1ccc2c(c1)OC(F)C(=O)N2Cc1ncc(Cl)cn1 ; NC(=O)c1c(CC2CC2)nc2c(cnn2C2(C(F)(F)F)CC2)c1-c1ccc2c(c1)OC(F)C(=O)N2Cc1ncc(Cl)cn1]

4-[(S)-4-[(5-chloro-2-pyrimidinyl)methyl]-2-fluoro-3-oxo-3,4-dihydro-2H-1,4-benzoxazin-7-yl]-6-(cyclopropylmethyl)-1-[1-(trifluoromethyl)cyclopropyl]-1,2,7-triaza-1H-indene-5-carboxamide which was synthesized following the similar route of Example 51 (60 mg) was purified by SFC (system: Waters SFC 150; Column name: REGIS (R,R) WHELK-01; Column size: 250 mm*25 mm, 10 μm; Mobile Phase A: Supercritical CO₂; Mobile Phase B: EtOH (+IPA (0.1% NH₃H₂O)); Gradient: A/B=60/40; Flow rate: 120 mL/min; Column Temp: RT) to afford 6-(cyclopropylmethyl)-4-[(S)-2-fluoro-4-[(5-fluoro-2-pyrimidinyl)methyl]-3-oxo-3,4-dihydro-2H-1,4-benzoxazin-7-yl]-1-[1-(5-fluoro-2-pyridyl)cyclopropyl]-1,2,7-triaza-1H-indene-5-carboxamide and 6-(cyclopropylmethyl)-4-[(R)-2-fluoro-4-[(5-fluoro-2-pyrimidinyl)methyl]-3-oxo-3,4-dihydro-2H-1,4-benzoxazin-7-yl]-1-[1-(5-fluoro-2-pyridyl)cyclopropyl]-1,2,7-triaza-1H-indene-5-carboxamide.

Compound 361, enantiomer 1 (14.42 mg, 24.03%): Rt=1.93 min. ¹H NMR (400 MHz, CDCl₃) δ 8.70 (s, 2H), 7.92 (s, 1H), 7.49 (d, J=1.6 Hz, 1H), 7.35 (dd, J=1.6, 8.4 Hz, 1H), 7.06 (d, J=8.6 Hz, 1H), 6.30 (d, J=51.6 Hz, 1H), 5.65-5.56 (m, 2H), 5.40 (br s, 1H), 5.29 (d, J=16.8 Hz, 1H), 2.96 (d, J=6.8 Hz, 2H), 1.81-1.76 (m, 2H), 1.71 (br s, 2H), 1.37-1.31 (m, 1H), 0.56-0.49 (m, 2H), 0.37-0.35 (m, 2H). ¹⁹F NMR (376 MHz, CDCl₃) δ −72.51, −128.00. LC-MS: m/z 616.1 (M+H)⁺.

Compound 362, enantiomer 2 (14.24 mg, 23.73%): Rt=2.33 min. ¹H NMR (400 MHz, CDCl₃) δ 8.70 (s, 2H), 7.92 (s, 1H), 7.49 (s, 1H), 7.35 (br d, J=8.4 Hz, 1H), 7.06 (d, J=8.4 Hz, 1H), 6.30 (d, J=51.2 Hz, 1H), 5.64-5.57 (m, 2H), 5.41 (br s, 1H), 5.30 (d, J=17.6 Hz, 1H), 2.96 (d, J=6.8 Hz, 2H), 1.80-1.77 (m, 2H), 1.71 (br s, 2H), 1.37-1.32 (m, 1H), 0.56-0.50 (m, 2H), 0.36 (q, J=4.8 Hz, 2H). ¹⁹F NMR (376 MHz, CDCl₃) δ −72.51, −128.0. LC-MS: m/z 616.1 (M+H)⁺.

### 6-(cyclopropylmethyl)-4-[(S)-2-fluoro-4-[(5-fluoro-2-pyrimidinyl)methyl]-3-oxo-3,4-dihydro-2H-1,4-benzoxazin-7-yl]-1-[1-(trifluoromethyl)cyclopropyl]-1,2,7-triaza-1H-indene-5-carboxamide and 6-(cyclopropylmethyl)-4-[(R)-2-fluoro-4-[(5-fluoro-2-pyrimidinyl)methyl]-3-oxo-3,4-dihydro-2H-1,4-benzoxazin-7-yl]-1-[1-(trifluoromethyl)cyclopropyl]-1,2,7-triaza-1H-indene-5-carboxamide (Compound 363 and Compound 364)

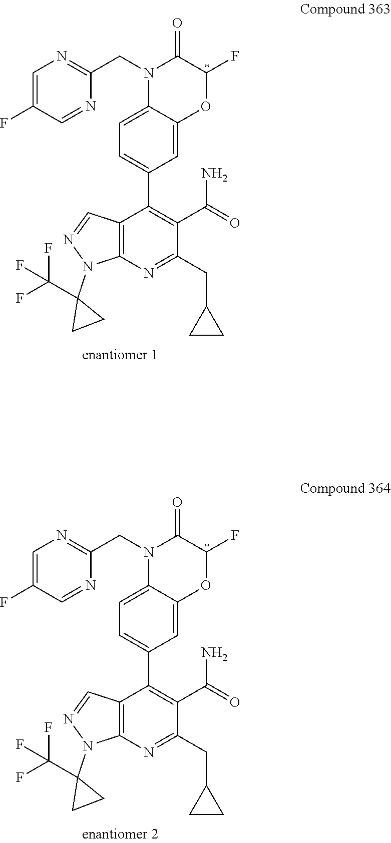 [SMILES: NC(=O)c1c(CC2CC2)nc2c(cnn2C2(C(F)(F)F)CC2)c1-c1ccc2c(c1)OC(F)C(=O)N2Cc1ncc(F)cn1 ; NC(=O)c1c(CC2CC2)nc2c(cnn2C2(C(F)(F)F)CC2)c1-c1ccc2c(c1)OC(F)C(=O)N2Cc1ncc(F)cn1]

6-(cyclopropylmethyl)-4-[2-fluoro-4-[(5-fluoro-2-pyrimidinyl)methyl]-3-oxo-3,4-dihydro-2H-1,4-benzoxazin-7-yl]-1-[1-(trifluoromethyl)cyclopropyl]-1,2,7-triaza-1H-indene-5-carboxamide which was synthesized following the similar route of Example 51 (70 mg) was purified by SFC (system: CASSH-SFC-17; Column name: DAICEL CHIRALCEL OJ; Column size: 250 mm*30 mm, 10 μm; Mobile Phase A: Supercritical CO₂; Mobile Phase B: MeOH (+0.1% NH₃H₂O); Gradient: A/B=70/30; Flow rate: 80 mL/min; Column Temp: 40° C.) to afford 6-(cyclopropylmethyl)-4-[(S)-2-fluoro-4-[(5-fluoro-2-pyrimidinyl)methyl]-3-oxo-3,4-dihydro-2H-1,4-benzoxazin-7-yl]-1-[1-(trifluoromethyl)cyclopropyl]-1,2,7-triaza-1H-indene-5-carboxamide and 6-(cyclopropylmethyl)-4-[(R)-2-fluoro-4-[(5-fluoro-2-pyrimidinyl)methyl]-3-oxo-3,4-dihydro-2H-1,4-benzoxazin-7-yl]-1-[1-(trifluoromethyl)cyclopropyl]-1,2,7-triaza-1H-indene-5-carboxamide.

Compound 363, enantiomer 1 (31.23 mg, 44.61%): Rt=1.67 min. ¹H NMR (400 MHz, CDCl₃) δ 8.60 (s, 2H), 7.91 (s, 1H), 7.48 (d, J=1.6 Hz, 1H), 7.34 (dd, J=8.4, 1.6 Hz, 1H), 7.07 (d, J=8.4 Hz, 1H), 6.23 (d, J=51.6 Hz, 1H), 5.76-5.67 (m, 1H), 5.66-5.56 (m, 1H), 5.50-5.40 (m, 1H), 5.35-5.25 (m, 1H), 2.95 (d, J=7.2 Hz, 2H), 1.83-1.74 (m, 2H), 1.74-1.67 (m, 2H), 1.40-1.28 (m, 1H), 0.58-0.46 (m, 2H), 0.40-0.30 (m, 2H). ¹⁹F NMR (376 MHz, CDCl₃) δ −72.50, −127.98, −138.16. LC-MS: m/z 600.2 (M+H)⁺.

Compound 364, enantiomer 2 (29.43 mg, 42.04%): Rt=2.01 min. ¹H NMR (400 MHz, CDCl₃) § 8.60 (s, 2H), 7.92 (s, 1H), 7.48 (d, J=2.0 Hz, 1H), 7.34 (dd, J=8.4, 2.0 Hz, 1H), 7.07 (d, J=8.4 Hz, 1H), 6.23 (d, J=51.6 Hz, 1H), 5.75-5.67 (m, 1H), 5.66-5.57 (m, 1H), 5.52-5.39 (m, 1H), 5.36-5.23 (m, 1H), 2.95 (d, J=6.8 Hz, 2H), 1.83-1.75 (m, 2H), 1.75-1.68 (m, 2H), 1.39-1.27 (m, 1H), 0.60-0.46 (m, 2H), 0.42-0.30 (m, 2H). ¹⁹F NMR (376 MHz, CDCl₃) δ −72.50, −127.98, −138.16. LC-MS: m/z 600.2 (M+H)⁺.

### Biological Assays

Amylin Receptor cAMP Assay

AMYRs are heterodimers of the class B calcitonin (CT) G-protein-coupled receptor (CTR) and receptor activity-modifying proteins (RAMPs). All three RAMPs can interact with the CTR and form AMY1, AMY2, AMY3 with RAMP1, RAMP2 and RAMP3, respectively. Like other class B1 GPCRs, the CT receptor family is canonically coupled to Gs-mediated cAMP production, and measurement of cAMP accumulation has been the primary assay used to determine peptide selectivity and potency.

To optimize functional activity directed toward Gas coupling, COS-7 cells were stably transfected with human calcitonin receptor (CTR) and RAMP3, simultaneously. 100× concentration of compound working solutions were prepared with 4-fold serial dilution in 384-well Echo LDV plate (Labcyte, Cat #LP-0200-BC). 200 nL/well 100× concentration of compound working solutions were moved to 384-well white microplate (Perkin Elmer, Cat #6007680) using Labcyte ECHO550. 1×10⁵ cells/mL COS-7/AMY3 cell suspensions prepared with assay buffer [HBSS containing 20 mM HEPES (Gibco, Cat #15630-080), 0.5 mM IBMX (Sigma, Cat #15879) and 0.1% Casein (Sigma, Cat #C4765)], 20 μL cell suspensions were added to each well of previous generated assay plate which already contains 200 nL compound at 100× concentration using ThermoFisher Multidrop Combi (2000 cells/well). Seal the plate and incubate at 37° C. with 5% CO₂ for 30 min.

After incubation, the CAMP assay signal was generated using cAMP dynamic 2 kit (Revvity, Cat #62AM4PEC). 10 μL cAMP-d2 working solution was added to each well, followed by 10 μL Anti-cAMP antibody-cryptate working solution which was added to each well using CERTUS FLEX LIQUID DISPENSER. The samples were then incubated at room temperature for 1 hour protected from light. the fluorescence was read at 665 and 615 nm with Reader PerkinElmer EnVision 2105.

$$\% Activity = 100 - ( mean RLU of test sample - mean RLU of positive control ) / ( mean RLU of vehicle control - mean RLU of positive control ) \times 100$$

Calcitonin Receptor (CTR) cAMP Assay

The calcitonin receptor (CTR) belongs to the subfamily of GPCRs known as the secretin or ‘B’ family of GPCRs. Like other class B1 GPCRs, the CT receptor family is canonically coupled to Gs-mediated cAMP production, and measurement of cAMP accumulation has been the primary assay used to determine peptide selectivity and potency.

To optimize functional activity directed toward Gas coupling, COS-7 cells were stably transfected with human calcitonin receptor (CTR) to create COS7-human CTR Clone #2 stable cell line. 100× concentration of compound working solutions were prepared with 4-fold serial dilution in 384-well Echo LDV plate (Labcyte, Cat #LP-0200-BC). 200 nL/well 100× concentration of compound working solutions were moved to 384-well white microplate (Perkin Elmer, Cat #6007680) using Labcyte ECHO550. 1×10⁵ cells/mL COS7-human CTR Clone #2 cell suspensions prepared with assay buffer [HBSS containing 20 mM HEPES (Gibco, Cat #15630-080), 0.5 mM IBMX (Sigma, Cat #15879) and 0.1% Casein (Sigma, Cat #C4765)], 20 μL cell suspensions were added to each well of previous generated assay plate which already contains 200 nL compound at 100× concentration using ThermoFisher Multidrop Combi (2000 cells/well). Seal the plate and incubate at 37° C. with 5% CO₂ for 30 min.

After incubation, the CAMP assay signal was generated using cAMP dynamic 2 kit (Revvity, Cat #62AM4PEC). 10 μL cAMP-d2 working solution was added to each well, followed by 10 μL Anti-cAMP antibody-cryptate working solution which was added to each well using CERTUS FLEX LIQUID DISPENSER. The samples were then incubated at room temperature for 1 hour protected from light. the fluorescence was read at 665 and 615 nm with Reader PerkinElmer EnVision 2105.

$$\% Activity = 100 - ( mean RLU of test sample - mean RLU of positive control ) / ( mean RLU of vehicle control - mean RLU of positive control ) \times 100$$

The activity of the tested compounds is provided in Table 3 below.

TABLE 3

| | Amylin Receptor cAMP | Calcitonin Receptor cAMP |
|---|---|---|
| Compound | Stimulation Activity: | Stimulation Activity: |
| No. | EC₅₀ (nM) | EC₅₀ (nM) |
| 101 | 48 | 4.58 |
| 102 | 0.76 | 0.625 |
| 103 | 0.04 | 0.0208 |
| 104 | 18 | 1.57 |
| 105 | 1.35 | 0.094 |
| 106 | 0.96 | 0.079 |
| 107 | 5.60 | 0.45 |
| 108 | 3.59 | 3.62 |
| 109 | 143 | 5.62 |
| 110 | 200 | 22.5 |
| 111 | 93.50 | 3.29 |
| 112 | 853 | 134 |
| 113 | 61 | 4.95 |
| 114 | >100000 | |
| 115 | 239.4 | |
| 116 | 385 | |
| 117 | 772 | 88.6 |
| 118 | 1300 | 160 |
| 119 | 537 | 55 |
| 120 | 333 | 38 |
| 121 | 370 | 71.5 |
| 122 | 19.30 | 3.99 |
| 123 | 573 | |
| 124 | 599 | |
| 125 | 577 | |
| 126 | 623 | 201 |
| 127 | 1420 | |
| 128 | >100000 | |
| 129 | 1222 | |
| 130 | 494 | 44.3 |
| 131 | 894 | 129 |
| 132 | 103 | |
| 133 | 104.2 | |
| 134 | 2150 | |
| 135 | 681 | |
| 136 | 1120 | |
| 141 | 50.70 | 1.8 |
| 142 | 0.96 | 0.045 |
| 143 | 2.03 | 0.084 |
| 144 | 6.92 | 0.36 |
| 145 | 1.41 | 0.17 |
| 146 | 0.88 | 0.14 |
| 147 | 2.11 | 0.27 |
| 148 | 2.21 | 0.24 |
| 149 | 0.60 | 0.029 |
| 150 | 2.41 | 0.012 |
| 151 | 0.21 | 0.018 |
| 152 | 1840 | 133 |
| 153 | 607 | 70.4 |
| 154 | 288 | 13 |
| 155 | 1120 | 46.7 |
| 156 | 26.2 | 0.63 |
| 157 | 11.2 | 0.66 |
| 158 | 39 | 1.69 |
| 159 | 381.9 | 32 |
| 160 | 2570 | |
| 161 | 5.77 | 0.045 |
| 162 | 0.761 | 0.034 |
| 163 | 28 | 3.86 |
| 164 | 2.4 | 0.11 |
| 165 | 146 | 1.45 |
| 166 | 16.6 | 1.57 |
| 167 | 520 | 10.1 |
| 168 | 2.34 | 0.053 |
| 169 | 4.18 | 0.13 |
| 170 | 1.07 | 0.07 |
| 171 | 114 | 1.5 |
| 172 | 4.12 | 0.095 |
| 173 | 9.5 | 0.17 |
| 174 | 4.64 | 0.085 |
| 175 | 7.91 | 0.6 |
| 176 | 17.6 | 0.89 |
| 177 | 6.44 | 0.41 |
| 178 | 20.7 | 7.76 |
| 179 | 35.3 | 1.45 |
| 180 | 11.3 | 0.5 |
| 181 | 3.62 | 0.21 |
| 182 | 11.5 | 0.52 |
| 183 | | 0.35 |
| 184 | 54.4 | 2.93 |
| 185 | 138 | 9.47 |
| 186 | 4.88 | 0.31 |
| 187 | | 1.59 |
| 188 | 57.4 | 3.62 |
| 189 | 54.4 | 3.25 |
| 190 | 84.4 | 4.65 |
| 191 | | 29.1 |
| 192 | 267 | 9.4 |
| 193 | | 744 |
| 194 | | 47.1 |
| 195 | 1.17 | 0.054 |
| 196 | 4.87 | 0.25 |
| 197 | 4.57 | 0.19 |
| 198 | 10.9 | 0.58 |
| 199 | 17.9 | 0.9 |
| 200 | 3.38 | 0.23 |
| 201 | 2 | 0.13 |
| 207 | 2.39 | 0.093 |
| 208 | 0.72 | 0.035 |
| 209 | 5.01 | 0.24 |
| 210 | 1.79 | 0.17 |
| 211 | 14.5 | 0.4 |
| 212 | 53.7 | 1.01 |
| 213 | | 1.68 |
| 214 | | 1.71 |
| 215 | 2.17 | 0.16 |
| 216 | 59.6 | 1.94 |
| 217 | 1.1 | 0.042 |
| 218 | 0.59 | 0.046 |
| 219 | 22.5 | 1.37 |
| 220 | 3.88 | 0.073 |
| 221 | 2.74 | 0.14 |
| 222 | 7.17 | 0.61 |
| 223 | 10.6 | 0.6 |
| 224 | 6.78 | 0.34 |
| 225 | 18.6 | 1.07 |
| 226 | 30.4 | 3.08 |
| 227 | 1.62 | 0.088 |
| 228 | 22.1 | 1.35 |
| 229 | 3.25 | 0.23 |
| 230 | | 2.13 |
| 231 | | 0.52 |
| 232 | 7.08 | 0.42 |
| 233 | 5.45 | 0.21 |
| 234 | 72.3 | 4.95 |
| 235 | 40 | 0.98 |
| 236 | 95.8 | 12.6 |
| 237 | 15.4 | 0.96 |
| 238 | | 0.75 |
| 239 | 4.56 | 0.22 |
| 240 | | 4.86 |
| 241 | 73 | 4.82 |
| 242 | 289 | 21.7 |
| 243 | 45.8 | 1.4 |
| 244 | 15.4 | 0.74 |
| 245 | 19.2 | 0.78 |
| 246 | 62.6 | 348 |
| 247 | | 2.05 |
| 248 | 18.5 | 0.46 |
| 249 | 193 | 14.4 |
| 250 | 48.7 | 4.41 |
| 251 | 284 | 10 |
| 252 | 71.1 | 3 |
| 253 | 5.65 | 0.28 |
| 254 | | 24 |
| 255 | 13.7 | 0.64 |
| 256 | | 1.32 |
| 257 | 4.49 | 0.11 |
| 258 | 2.6 | 0.085 |
| 259 | 13.1 | 0.35 |
| 260 | 5.43 | 0.32 |
| 261 | 7.93 | 0.31 |
| 262 | 0.92 | 0.029 |
| 263 | 0.67 | 0.077 |
| 264 | 0.42 | 0.027 |
| 265 | 2.09 | 0.061 |
| 266 | 1.9 | 0.068 |
| 267 | | 1.51 |
| 268 | 12.9 | 0.46 |
| 269 | | 15.3 |
| 270 | 9.22 | 0.42 |
| 271 | 2.85 | 0.12 |
| 272 | 2.22 | 0.16 |
| 273 | 3.57 | 0.2 |
| 274 | 3.82 | 0.19 |
| 275 | 2.26 | 0.11 |
| 276 | 1.8 | 0.18 |
| 278 | 4.65 | 0.27 |
| 279 | 60.1 | 3.27 |
| 280 | | 86.5 |
| 281 | 3.81 | 0.069 |
| 282 | 13.6 | 0.74 |
| 283 | | 34.5 |
| 284 | 14.6 | 0.2 |
| 285 | 3.59 | 0.19 |
| 286 | 499 | 21.4 |
| 287 | 2.4 | 0.093 |
| 288 | | 19.1 |
| 289 | 1.86 | 0.14 |
| 290 | 2.19 | 0.13 |
| 291 | | 2.33 |
| 292 | 0.46 | 0.03 |
| 293 | | 3.86 |
| 294 | | 4.01 |
| 295 | 201 | 5.27 |
| 296 | 2.87 | 0.17 |
| 297 | 4.41 | 0.42 |
| 298 | 13.7 | 1.31 |
| 299 | 75.6 | 1.13 |
| 300 | 104.9 | 6.52 |
| 301 | 52.4 | 1.26 |
| 302 | | >1000 |
| 303 | 3.68 | 0.21 |
| 304 | 51.3 | 1.11 |
| 305 | 30.6 | 0.56 |
| 306 | | 2.5 |
| 307 | 35.9 | 0.98 |
| 308 | 246.3 | 5.34 |
| 309 | 167.8 | 4.21 |
| 310 | 131.7 | 2.09 |
| 311 | 218.8 | 4.53 |
| 312 | 91.1 | 3.77 |
| 313 | 46.3 | 4.89 |
| 314 | 95.7 | 2.07 |
| 315 | 41.1 | 0.52 |
| 316 | 17 | 0.38 |
| 317 | 61.3 | 0.76 |
| 318 | 27.3 | 0.7 |
| 319 | 43.6 | 1.64 |
| 320 | 85.2 | 2.62 |
| 321 | 55.0 | 0.73 |
| 322 | 53.8 | 1.32 |
| 323 | 86.4 | 1.19 |
| 324 | 12.4 | 1.39 |
| 325 | 45.5 | 1.82 |
| 326 | 48.8 | 3.13 |
| 327 | 10.2 | 0.27 |
| 328 | | 33.5 |
| 329 | 2.98 | 0.67 |
| 330 | 12.4 | 1.7 |
| 331 | 5.78 | 0.65 |
| 332 | 99.0 | 2.3 |
| 333 | 4.43 | 0.18 |
| 334 | 6.51 | 0.97 |
| 335 | 14.2 | 1.18 |
| 336 | 13.5 | 1.19 |
| 337 | 13.3 | 1.79 |
| 338 | 37.8 | 2.46 |
| 339 | 293.4 | 5.38 |
| 340 | | 57.3 |
| 341 | | 20.4 |
| 342 | 18.4 | 0.89 |
| 343 | 4.3 | 0.12 |
| 344 | 13.6 | 0.45 |
| 345 | 8.49 | 0.24 |
| 346 | 45.3 | 0.72 |
| 347 | 6.33 | 0.34 |
| 348 | 44.6 | 1.22 |
| 349 | 4.97 | 0.19 |
| 350 | 25.1 | 0.64 |
| 351 | 58.6 | 2.58 |
| 352 | | 12.9 |
| 353 | 23.5 | 1.03 |
| 354 | 1.62 | 0.088 |
| 355 | 6.3 | 0.13 |
| 356 | 143.4 | 2.89 |
| 357 | 180.1 | 3.15 |
| 358 | 8.2 | 0.095 |
| 359 | 4.58 | 0.23 |
| 360 | 246 | 5.79 |
| 361 | | 21.7 |
| 362 | 15.5 | 0.72 |
| 363 | | 52.4 |
| 364 | 57.8 | 1.63 |

1-31. (canceled)

32. A compound selected from the group consisting of: 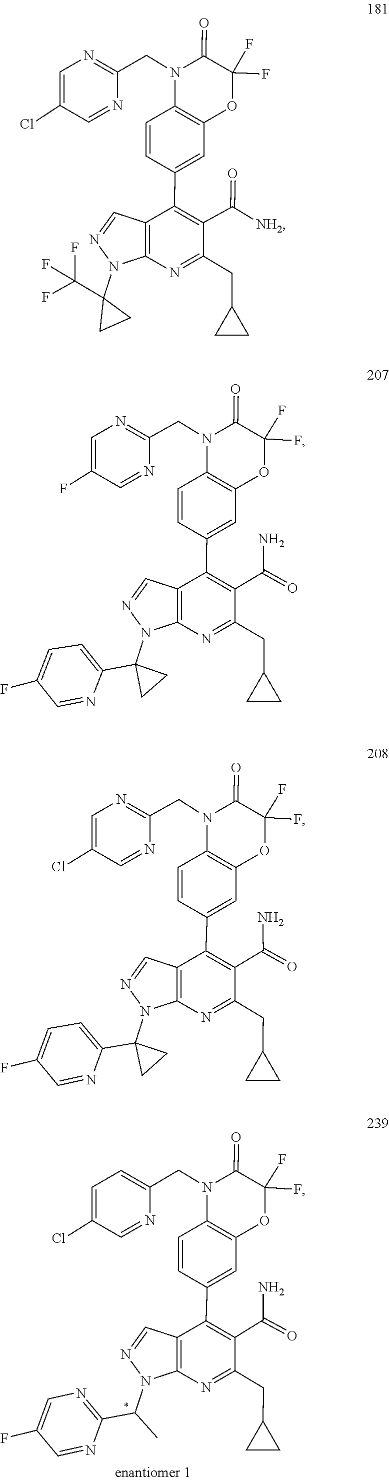 [SMILES: NC(=O)c1c(CC2CC2)nc2c(cnn2C2(C(F)(F)F)CC2)c1-c1ccc2c(c1)OC(F)(F)C(=O)N2Cc1ncc(Cl)cn1 ; NC(=O)c1c(CC2CC2)nc2c(cnn2C2(c3ccc(F)cn3)CC2)c1-c1ccc2c(c1)OC(F)(F)C(=O)N2Cc1ncc(F)cn1 ; NC(=O)c1c(CC2CC2)nc2c(cnn2C2(c3ccc(F)cn3)CC2)c1-c1ccc2c(c1)OC(F)(F)C(=O)N2Cc1ncc(Cl)cn1 ; CC(c1ncc(F)cn1)n1ncc2c(-c3ccc4c(c3)OC(F)(F)C(=O)N4Cc3ccc(Cl)cn3)c(C(N)=O)c(CC3CC3)nc21] 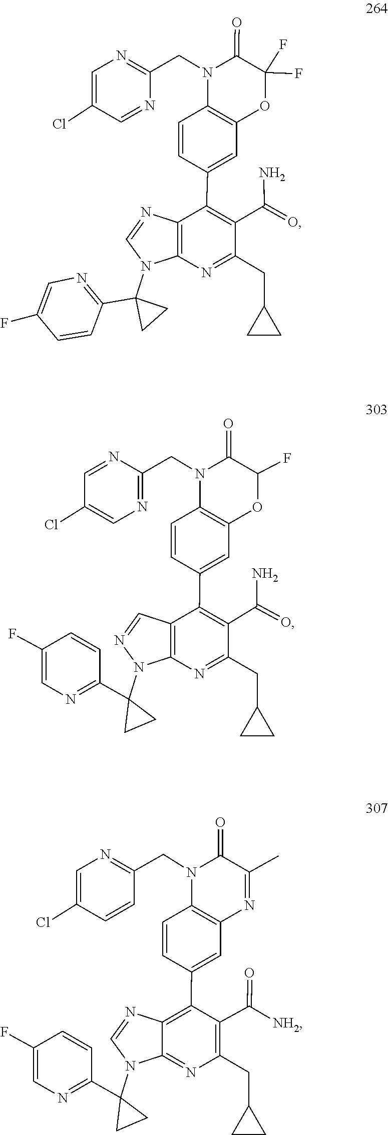 [SMILES: NC(=O)c1c(CC2CC2)nc2c(ncn2C2(c3ccc(F)cn3)CC2)c1-c1ccc2c(c1)OC(F)(F)C(=O)N2Cc1ncc(Cl)cn1 ; NC(=O)c1c(CC2CC2)nc2c(cnn2C2(c3ccc(F)cn3)CC2)c1-c1ccc2c(c1)OC(F)C(=O)N2Cc1ncc(Cl)cn1 ; Cc1nc2cc(-c3c(C(N)=O)c(CC4CC4)nc4c3ncn4C3(c4ccc(F)cn4)CC3)ccc2n(Cc2ccc(Cl)cn2)c1=O] 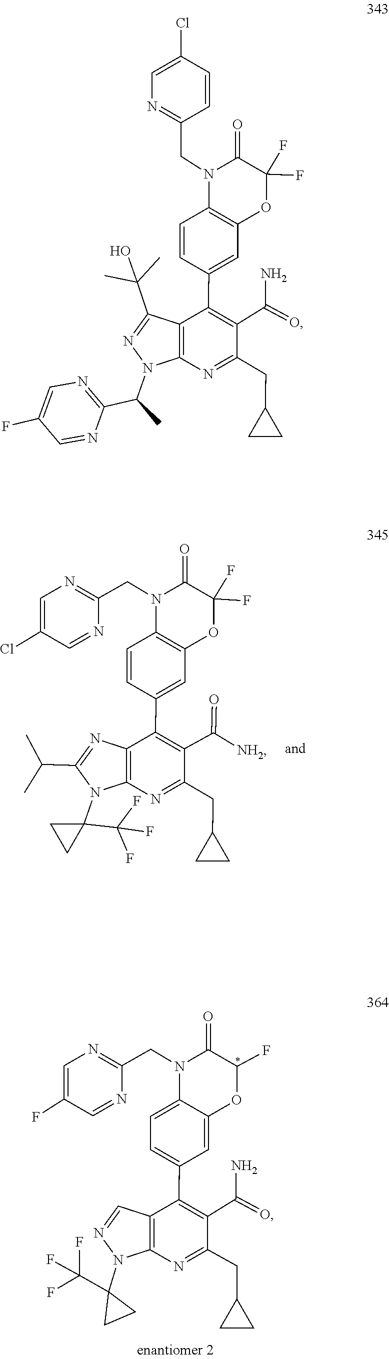 [SMILES: C[C@@H](c1ncc(F)cn1)n1nc(C(C)(C)O)c2c(-c3ccc4c(c3)OC(F)(F)C(=O)N4Cc3ccc(Cl)cn3)c(C(N)=O)c(CC3CC3)nc21 ; CC(C)c1nc2c(-c3ccc4c(c3)OC(F)(F)C(=O)N4Cc3ncc(Cl)cn3)c(C(N)=O)c(CC3CC3)nc2n1C1(C(F)(F)F)CC1 ; NC(=O)c1c(CC2CC2)nc2c(cnn2C2(C(F)(F)F)CC2)c1-c1ccc2c(c1)OC(F)C(=O)N2Cc1ncc(F)cn1]
or a pharmaceutically acceptable salt thereof.

33. A pharmaceutical composition comprising a compound of claim 32, or a pharmaceutically acceptable salt thereof, and a pharmaceutically acceptable excipient.

34. The compound of claim 32, wherein the compound is selected from the group consisting of:

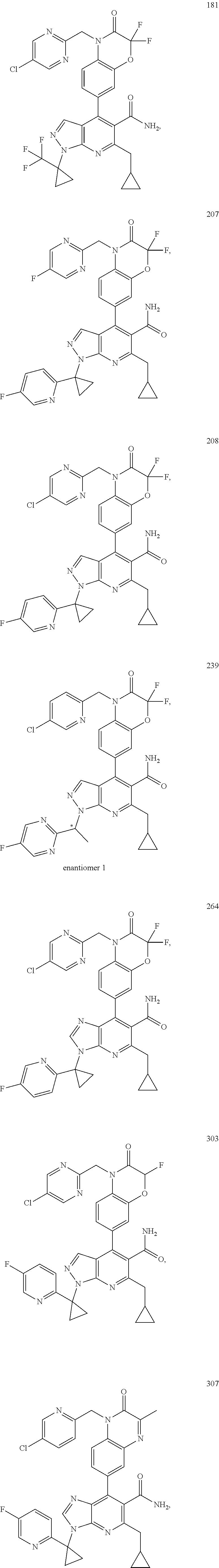

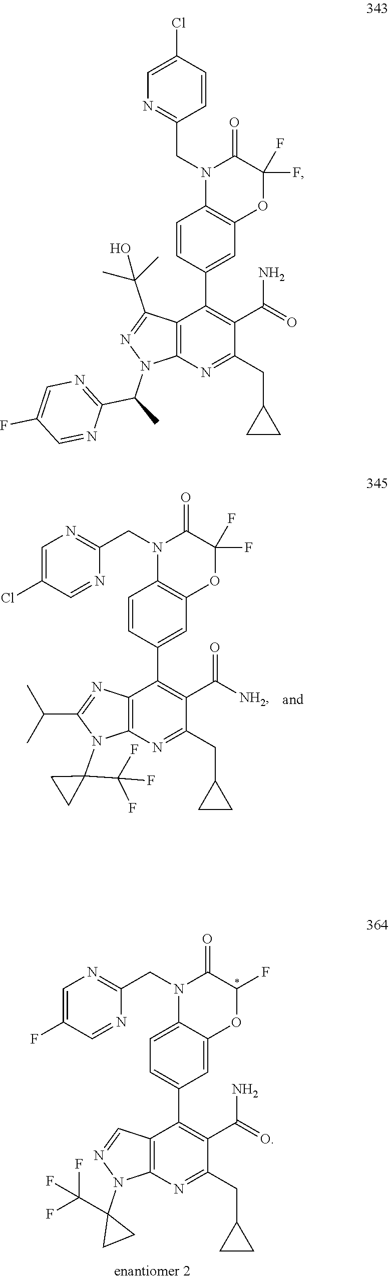

35. A pharmaceutical composition comprising a compound of claim 34, and a pharmaceutically acceptable excipient.

36. The compound of claim 32, wherein the compound is: 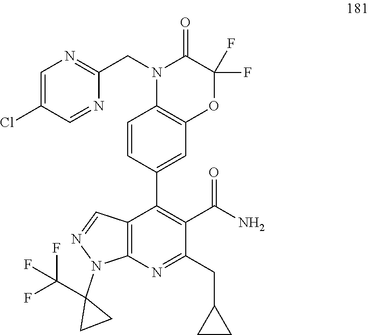 [SMILES: NC(=O)c1c(CC2CC2)nc2c(cnn2C2(C(F)(F)F)CC2)c1-c1ccc2c(c1)OC(F)(F)C(=O)N2Cc1ncc(Cl)cn1]
or a pharmaceutically acceptable salt thereof.

37. A pharmaceutical composition comprising a compound of claim 36, or a pharmaceutically acceptable salt thereof, and a pharmaceutically acceptable excipient.

38. The compound of claim 32, wherein the compound is: 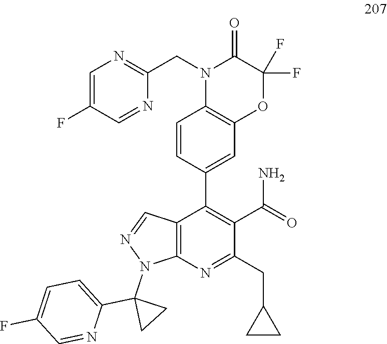 [SMILES: NC(=O)c1c(CC2CC2)nc2c(cnn2C2(c3ccc(F)cn3)CC2)c1-c1ccc2c(c1)OC(F)(F)C(=O)N2Cc1ncc(F)cn1]
or a pharmaceutically acceptable salt thereof.

39. A pharmaceutical composition comprising a compound of claim 38, or a pharmaceutically acceptable salt thereof, and a pharmaceutically acceptable excipient.

40. The compound of claim 32, wherein the compound is: 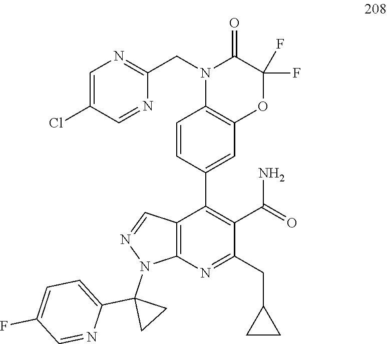 [SMILES: NC(=O)c1c(CC2CC2)nc2c(cnn2C2(c3ccc(F)cn3)CC2)c1-c1ccc2c(c1)OC(F)(F)C(=O)N2Cc1ncc(Cl)cn1]
or a pharmaceutically acceptable salt thereof.

41. A pharmaceutical composition comprising a compound of claim 40, or a pharmaceutically acceptable salt thereof, and a pharmaceutically acceptable excipient.

42. The compound of claim 32, wherein the compound is: 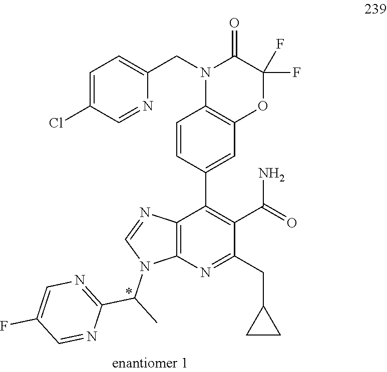 [SMILES: CC(c1ncc(F)cn1)n1cnc2c(-c3ccc4c(c3)OC(F)(F)C(=O)N4Cc3ccc(Cl)cn3)c(C(N)=O)c(CC3CC3)nc21]
or a pharmaceutically acceptable salt thereof.

43. A pharmaceutical composition comprising a compound of claim 42, or a pharmaceutically acceptable salt thereof, and a pharmaceutically acceptable excipient.

44. The compound of claim 32, wherein the compound is: 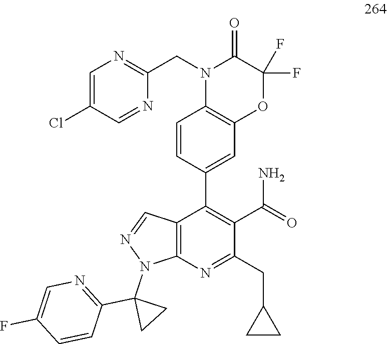 [SMILES: NC(=O)c1c(CC2CC2)nc2c(cnn2C2(c3ccc(F)cn3)CC2)c1-c1ccc2c(c1)OC(F)(F)C(=O)N2Cc1ncc(Cl)cn1]
or a pharmaceutically acceptable salt thereof.

45. A pharmaceutical composition comprising a compound of claim 44, or a pharmaceutically acceptable salt thereof, and a pharmaceutically acceptable excipient.

46. The compound of claim 32, wherein the compound is: 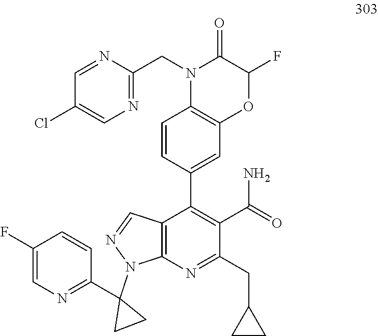 [SMILES: NC(=O)c1c(CC2CC2)nc2c(cnn2C2(c3ccc(F)cn3)CC2)c1-c1ccc2c(c1)OC(F)C(=O)N2Cc1ncc(Cl)cn1]
or a pharmaceutically acceptable salt thereof.

47. A pharmaceutical composition comprising a compound of claim 46, or a pharmaceutically acceptable salt thereof, and a pharmaceutically acceptable excipient.

48. The compound of claim 32, wherein the compound is: 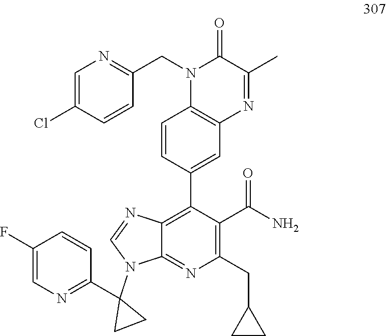 [SMILES: Cc1nc2cc(-c3c(C(N)=O)c(CC4CC4)nc4c3ncn4C3(c4ccc(F)cn4)CC3)ccc2n(Cc2ccc(Cl)cn2)c1=O]
or a pharmaceutically acceptable salt thereof.

49. A pharmaceutical composition comprising a compound of claim 48, or a pharmaceutically acceptable salt thereof, and a pharmaceutically acceptable excipient.

50. The compound of claim 32, wherein the compound is: 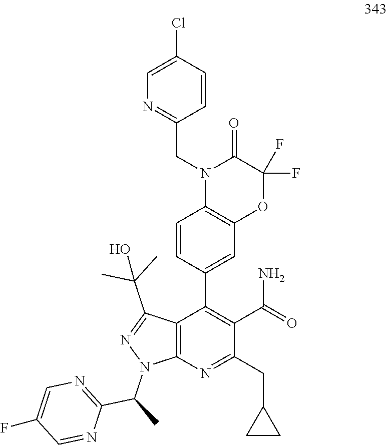 [SMILES: C[C@@H](c1ncc(F)cn1)n1nc(C(C)(C)O)c2c(-c3ccc4c(c3)OC(F)(F)C(=O)N4Cc3ccc(Cl)cn3)c(C(N)=O)c(CC3CC3)nc21]
or a pharmaceutically acceptable salt thereof.

51. A pharmaceutical composition comprising a compound of claim 50, or a pharmaceutically acceptable salt thereof, and a pharmaceutically acceptable excipient.

52. The compound of claim 32, wherein the compound is: 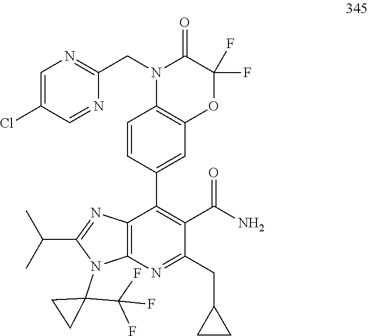 [SMILES: CC(C)c1nc2c(-c3ccc4c(c3)OC(F)(F)C(=O)N4Cc3ncc(Cl)cn3)c(C(N)=O)c(CC3CC3)nc2n1C1(C(F)(F)F)CC1]
or a pharmaceutically acceptable salt thereof.

53. A pharmaceutical composition comprising a compound of claim 52, or a pharmaceutically acceptable salt thereof, and a pharmaceutically acceptable excipient.

54. The compound of claim 32, wherein the compound is: 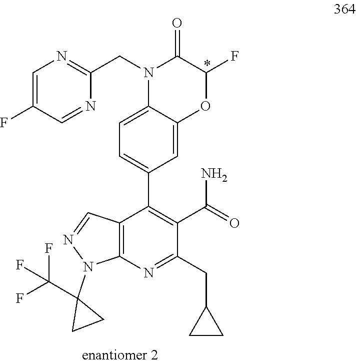 [SMILES: NC(=O)c1c(CC2CC2)nc2c(cnn2C2(C(F)(F)F)CC2)c1-c1ccc2c(c1)OC(F)C(=O)N2Cc1ncc(F)cn1]
or a pharmaceutically acceptable salt thereof.

55. A pharmaceutical composition comprising a compound of claim 54, or a pharmaceutically acceptable salt thereof, and a pharmaceutically acceptable excipient.
